(19) United States
(12) Patent Application Publication
(10) Pub. No.: US 2024/0368132 A1
(43) Pub. Date: Nov. 7, 2024

(54) ROCK2 INHIBITORS AND USES THEREOF

(71) Applicants: President and Fellows of Harvard College, Cambridge, MA (US); Trustees of Boston University, Boston, MA (US)

(72) Inventors: Joel D. Moore, Cambridge, MA (US); Christopher S. Chen, Boston, MA (US)

(73) Assignees: President and Fellows of Harvard College, Cambridge, MA (US); Trustees of Boston University, Boston, MA (US)

(21) Appl. No.: 18/292,051

(22) PCT Filed: Jul. 26, 2022
(86) PCT No.: PCT/US2022/038271

§ 371 Date: Jan. 25, 2024

Related U.S. Application Data
(60) Provisional application No. 63/346,144, filed on May 26, 2022.
(60) Provisional application No. 63/225,695, filed on Jul. 26, 2021.

(51) Int. Cl. C07D 403/14; A61K 31/416; A61K 31/4178; A61K 31/422; A61K 31/4245; A61K 31/437; A61K 31/4439; A61K 31/497; A61K 31/506; C07D 401/14; C07D 403/12; C07D 413/12; C07D 413/14; C07D 471/04; C07D 495/04; C07D 513/04

(52) U.S. Cl. CPC C07D 403/14; A61K 31/416; A61K 31/4178; A61K 31/422; A61K 31/4245; A61K 31/437; A61K 31/4439; A61K 31/497; A61K 31/506; C07D 401/14; C07D 403/12; C07D 413/12; C07D 413/14; C07D 471/04; C07D 495/04; C07D 513/04

(57) ABSTRACT

The present disclosure provides compounds of Formula (I) and (II), which may be ROCK2 inhibitors. The present disclosure also provides pharmaceutical compositions and kits comprising the compounds, and methods of treating or preventing diseases and disorders associated with ROCK2 (e.g., fibrotic disease, autoimmune disease, inflammatory-fibrotic condition, inflammatory condition, edema, ophthalmic disease, cardiovascular disease, central nervous system disorder, cancer) by administering to a subject in need thereof the compounds or pharmaceutical compositions.

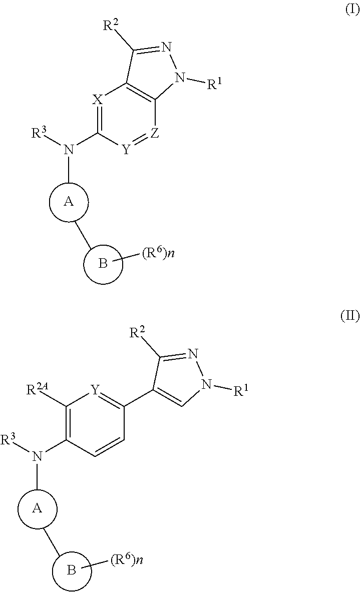

### RELATED APPLICATIONS

This application claims priority under 35 U.S.C. § 119(e) to U.S. Provisional Applications, U.S. Ser. No. 63/225,695, filed Jul. 26, 2021; and U.S. Ser. No. 63/346,144, filed May 26, 2022, each of which is incorporated herein by reference in its entirety.

### BACKGROUND

Rho-Kinase (ROCK) is a coiled-coil forming serine-threonine protein kinase family and exists in two isoforms, ROCK1 and ROCK2. ROCK has been identified as an effector molecule of RhoA, a small GTP-binding protein (G protein). Both proteins are ubiquitously expressed across tissues and play key roles in multiple cellular signalling pathways. Upon receptor activation RhoA activates ROCK that in turn controls several cellular functions including cell migration, cell adhesion, actin reorganisation, cytokinesis, and smooth muscle contraction.

Accordingly, ROCK inhibitors have potential therapeutic applicability in a wide variety of pathological conditions.

### SUMMARY OF THE DISCLOSURE

The present disclosure stems from the recognition that the unique structure and function of ROCK provides an opportunity for the design of ROCK2 inhibitors (e.g., selective ROCK2 inhibitors) useful in the treatment of a wide variety of diseases. For example, ROCK is a critical mediator of both biomechanical (tissue stiffness) and biochemical (TGF-0 mediated) pathways involved in the dysregulated activation of myofibroblasts, the cells thought to underlie the pathogenesis of fibrotic disease. Aberrant expression and activation of ROCK results in the sustained presence of activated myofibroblasts and excessive extracelluar matrix production, leading to tissue fibrosis. Recent studies show that selective inhibition of ROCK2 results in the inhibition of the production of pathogenic cytokine IL-17 in immune cells. As a result, selective inhibitors of ROCK2 may be effective in the treatment of fibrotic disease, among others. Thus, the disclosed compounds provide new compositions and methods for the treatment of diseases and disorders associated with ROCK2 (e.g., associated with increased ROCK2 activity) (e.g., fibrotic disorder, autoimmune disease, inflammatory condition, edema, ophthalmic disease, cardiovascular disease, central nervous system disorder, cancer).

In one aspect, provided are compounds of Formula (I):

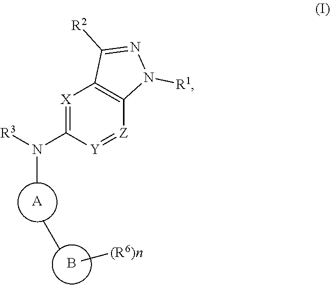

and pharmaceutically acceptable salts, co-crystals, tautomers, stereoisomers, solvates, hydrates, polymorphs, isotopically enriched compounds, and prodrugs thereof, wherein the moieties and variables included in Formula (I) are as described herein.

In another aspect, provided are compounds of Formula (II):

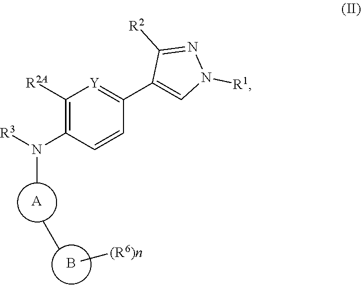

and pharmaceutically acceptable salts, co-crystals, tautomers, stereoisomers, solvates, hydrates, polymorphs, isotopically enriched compounds, and prodrugs thereof, wherein the moieties and variables included in Formula (II) are as described herein.

In another aspect, provided are pharmaceutical compositions comprising a provided compound and optionally a pharmaceutically acceptable excipient.

In another aspect, provided are methods of treating a disease or disorder associated with ROCK2 in a subject in need thereof, the method comprising administering to the subject an effective amount of a provided compound or pharmaceutical composition.

In another aspect, provided are methods of preventing a disease or disorder associated with ROCK2 in a subject in need thereof, the method comprising administering to the subject an effective amount of a provided compound or pharmaceutical composition.

In certain embodiments, the disease or disorder associated with ROCK2 is edema (e.g., lymphedema).

In another aspect, provided are methods of inhibiting the activity of ROCK2, the method comprising contacting ROCK2 with an effective amount of a provided compound or pharmaceutical composition.

In another aspect, the present disclosure provides methods of screening a library of compounds comprising performing an assay on a provided compound and an additional compound, wherein the additional compound is different from the provided compound.

In another aspect, provided are kits comprising a provided compound or pharmaceutical composition and instructions for using the provided compound or pharmaceutical composition.

The details of certain embodiments of the invention are set forth in the Detailed Description of Certain Embodiments, as described below. Other features, objects, and advantages of the invention will be apparent from the Definitions, Examples, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroCl₁₋₁₂ alkyl”). In some embodiments, a heteroalkyl group is a saturated group having 1 to 10 carbon atoms and 1 or more heteroatoms within the parent chain (“heteroCl₁₋₁₀ alkyl”). In some embodiments, a heteroalkyl group is a saturated group having 1 to 8 carbon atoms and 1 or more heteroatoms within the parent chain (“heteroC₁₋₈alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified [symbol] (e.g., —CH═CHCH₃ or [symbol] ) may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3-to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 7 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NRbC(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂OP(═O)(R^{aa})₂—OP(═O)(OR^{cc})₂, —P(═O)(N(R^{bb})₂)₂, —OP(═O)(N(R^{bb})₂)₂, NR^{bb}P(═O)(R^{aa})₂, —NR^{bb}P(═O)(OR^{cc})₂—NR^{bb}P(═O)(N(R^{bb})₂)₂, —P(Rc)₂, —P(OR^{cc})₂, —P(R^{cc})₃⁺X⁻, —P(OR^{cc})+X⁻, —P(R^{cc})₄, —P(OR^{cc})₄, —OP(R^{cc})₂, —OP(R^{cc})₃⁺X⁻, —OP(OR^{cc})₂, —OP(OR^{cc})⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, —NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃0.10 carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SRC, —C(═S)SRC, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X-is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R)₂, —N(R)₂, —N(R)₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R)₂, —OC(═O)N(R)₂, —NR^{ff}C(═O)R^{ee}, —NR^{ff}CO₂R^{ee}, —NRC(═O)N(R)₂, —C(═NR)OR^{ee}, —OC(═NR^{ff})R^{ee}, —OC(═NR)OR^{ee}, —C(═NR)N(R)₂, —OC(═NR)N(R)₂, —C(═NR)N(R)₂, —NRSO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R)₂, —C(═O)SR^{ee}, —C(═S)SR^{ee}, —SC(═S)SR^{ee}, —P(═O)(OR^{ee})₂, —P(═O)(R^{ee})₂—OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁-6 perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═o)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═o)S(C₁₋₆ alkyl), —C(═S)SC₁₋₆ alkyl, —SC(═S)SC₁₋₆ alkyl, —P(═o)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})⁺X⁻, —OP(OR^{cc})₂, —OP(OR^{cc})₃⁺X⁻, —OP(═O)(R^{aa})₂, —OP(═O)(OR^{aa})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc}C are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}—NR^{bb}CO₂R^{aa}—NR^{bb}C(═O)N(R^{bb})₂—NR^{bb}C(═NR^{bb})N(R^{bb})₂, —NR^{bb}SO₂R^{aa}, NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di- heteroaliphaticamino, mono- or di-alkylamino, mono- or di- heteroalkylamino, mono- or di-arylamino, or mono- or di-heteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂-10 alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd}, edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂Ra^{a}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃X-, —P(OR^{cc})₂, —P(OR^{cc})₃X-, —P(═O)(R^{aa})₂, —P(═O)(OR^{cc})₂, and —P(═O)(N(R^{bb})₂)₂, wherein X-, R^{aa}, R^{bb}, and R^{cc}C are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{aa})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{d} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF4⁻, PF⁻, AsF₆⁻, SbF₆⁻, B[3,5-(CF₃)₂C₆H₃]₄]⁻, B(C₆F5)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, and Claims. The invention is not intended to be limited in any manner by the above exemplary listing of substituents.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease or disorder.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease or disorder described herein. In some embodiments, treatment may be administered after one or more signs or symptoms of the disease or disorder have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The term “prevent,” “preventing,” or “prevention” refers to a prophylactic treatment of a subject who is not and was not with a disease or disorder but is at risk of developing the disease or disorder or who was with a disease or disorder, is not with the disease or disorder, but is at risk of regression of the disease or disorder. In certain embodiments, the subject is at a higher risk of developing the disease or disorder or at a higher risk of regression of the disease or disorder than an average healthy member of a population of subjects.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. For example, in treating cancer, an effective amount of an inventive composition may prevent tumor regrowth, reduce the tumor burden, or stop the growth or spread of a tumor. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for ROCK2 inhibition (e.g., at least 5%, at least 10%, at least 20%, at least 30%, at least 40%, at least 50%, at least 60%, at least 70%, at least 80%, at least 90%, at least 95%, or at least 99% inhibition of the activity of ROCK2). In certain embodiments, a therapeutically effective amount is an amount sufficient for treating a disease or disorder associated with ROCK2. In certain embodiments, a therapeutically effective amount is an amount sufficient for ROCK2 inhibition and treating a disease or disorder associated with ROCK2.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for ROCK2 inhibition. In certain embodiments, a prophylactically effective amount is an amount sufficient for treating a disease or disorder associated with ROCK2. In certain embodiments, a prophylactically effective amount is an amount sufficient for ROCK2 inhibition and treating a disease or disorder associated with ROCK2.

As used herein, the term “inhibit” or “inhibition” in the context of enzymes, for example, in the context of ROCK2, refers to a reduction in the activity of the enzyme. In some embodiments, the term refers to a reduction of the level of enzyme activity, e.g., ROCK2 activity, to a level that is statistically significantly lower than an initial level, which may, for example, be a baseline level of enzyme activity. In some embodiments, the term refers to a reduction of the level of enzyme activity, e.g., ROCK2 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity. In some embodiments, the term refers to a reduction of the level of enzyme activity, e.g., ROCK1 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ophthalmic cancer (e.g., intraocular melanoma, retinoblastoma); familiar hypereosinophilia; gall bladder cancer (e.g., gall bladder carcinoma); gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematological cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma, hepatic carcinoma); lung cancer (e.g., bronchogenic carcinoma, small cell lung cancer (SCLC), non-small cell lung cancer (NSCLC), adenocarcinoma of the lung, squamous carcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); cancer of the peritoneum; pinealoma; pituitary cancer; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer (e.g., salivary gland carcinoma); skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat and/or ameliorate a disease or disorder. Therapeutic agents, as disclosed herein, may be biologics or small molecule therapeutics, or combinations thereof.

The term “chemotherapeutic agent” refers to a therapeutic agent known to be of use in chemotherapy for cancer.

### DETAILED DESCRIPTION OF CERTAIN EMBODIMENTS OF THE DISCLOSURE

Provided herein are compounds that are ROCK inhibitors (e.g., ROCK2 inhibitors). The compounds possess advantageous properties, such as selective inhibition of ROCK2, that allow the compounds to be useful as therapeutic agents. In one aspect, the provided ROCK inhibitors are compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. In another aspect, the provided ROCK inhibitors are compounds of Formula (II), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with ROCK2 in a subject in need thereof.

The compounds described herein may interact with (e.g., bind) ROCK2. As described herein, the therapeutic effect may be a result of inhibition, modulation, binding, and/or modification of ROCK2 by the compounds described herein. The compounds may be provided for use in any composition, kit, or method described herein as a pharmaceutically acceptable salt, co-crystal, tautomer, stereoisomer, solvate, hydrate, polymorph, isotopically enriched compound, or prodrug thereof.

### Compounds

In one aspect, disclosed is a compound of Formula (I):

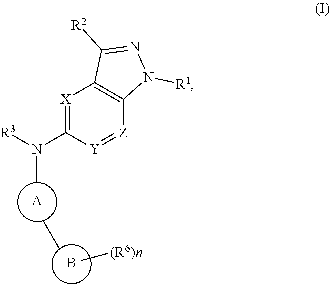

or a pharmaceutically acceptable salt, co-crystal, tautomer, stereoisomer, solvate, hydrate, polymorph, isotopically enriched compound, or prodrug thereof, wherein:
R¹ is hydrogen, substituted or unsubstituted alkyl, or a nitrogen protecting group;
R² is hydrogen, halogen, —CN, substituted or unsubstituted alkyl, or substituted or unsubstituted carbocyclyl;
X is CR⁷ or N;
Y is CR⁸ or N;
Z is CR⁹ or N;
R³ is hydrogen, substituted or unsubstituted alkyl, or a nitrogen protecting group;
A is

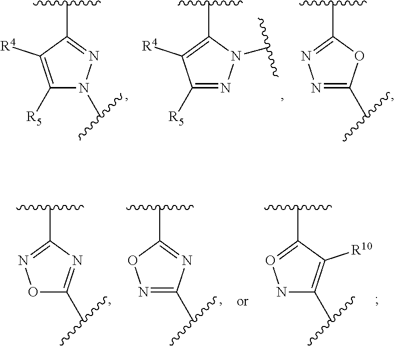

R⁴ is hydrogen, halogen, substituted or unsubstituted alkyl, substituted or unsubstituted carbocyclyl, substituted or unsubstituted aryl, or substituted or unsubstituted heteroaryl;
R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl;
B is aryl, heterocyclyl, heteroaryl, or carbocyclyl;
each R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, oxo, —OR^{A}, —N₃, —N(RA)₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A} or —S(O)₂R^{A};
n is 1, 2, 3, 4, or 5;
each R⁷, R⁸, and R⁹ is independently hydrogen, halogen, —CN, or substituted or unsubstituted alkyl;
R¹⁰ is hydrogen, halogen, substituted or unsubstituted alkyl, substituted or unsubstituted carbocyclyl, substituted or unsubstituted aryl, or substituted or unsubstituted heteroaryl; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

In certain embodiments, disclosed is a compound of Formula (I):

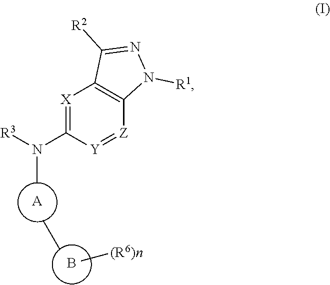

or a pharmaceutically acceptable salt, co-crystal, tautomer, stereoisomer, solvate, hydrate, polymorph, isotopically enriched compound, or prodrug thereof, wherein:
R¹ is hydrogen, substituted or unsubstituted alkyl, or a nitrogen protecting group;
R² is hydrogen, halogen, —CN, substituted or unsubstituted alkyl, or substituted or unsubstituted carbocyclyl;
X is CR⁷ or N;
Y is CR⁸ or N;
Z is CR⁹ or N;
R³ is hydrogen, substituted or unsubstituted alkyl, or a nitrogen protecting group;
A is

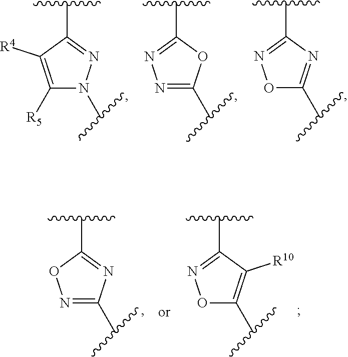

R⁴ is hydrogen, halogen, substituted or unsubstituted alkyl, substituted or unsubstituted carbocyclyl, substituted or unsubstituted aryl, or substituted or unsubstituted heteroaryl; R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl;
B is aryl, heterocyclyl, heteroaryl, or carbocyclyl;
each R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, oxo, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A} or —S(O)₂R^{A};
n is 1, 2, 3, 4, or 5;
each R⁷, R⁸, and R⁹ is independently hydrogen, halogen, —CN, or substituted or unsubstituted alkyl;
R¹⁰ is hydrogen, halogen, substituted or unsubstituted alkyl, substituted or unsubstituted carbocyclyl, substituted or unsubstituted aryl, or substituted or unsubstituted heteroaryl; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

In another aspect, disclosed is a compound of Formula (II):

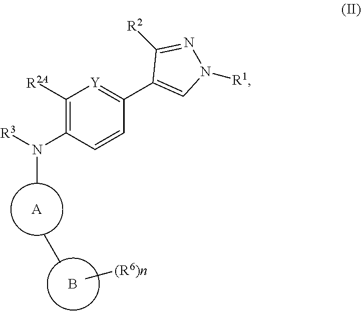

or a pharmaceutically acceptable salt, co-crystal, tautomer, stereoisomer, solvate, hydrate, polymorph, isotopically enriched compound, or prodrug thereof, wherein:
R¹ is hydrogen, substituted or unsubstituted alkyl, or a nitrogen protecting group;
R² is hydrogen, halogen, —CN, substituted or unsubstituted alkyl, or substituted or unsubstituted carbocyclyl;
R^{2A} is hydrogen, halogen, —CN, or substituted or unsubstituted alkyl;
Y is CR⁸ or N;
R³ is hydrogen, substituted or unsubstituted alkyl, or a nitrogen protecting group;
A is

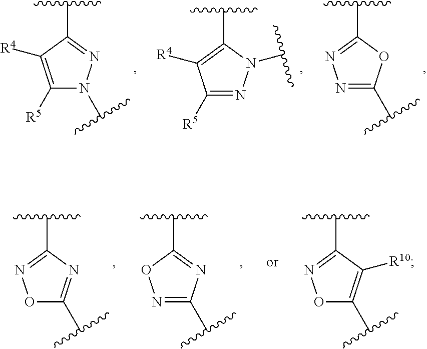

R⁴ is hydrogen, halogen, substituted or unsubstituted alkyl, substituted or unsubstituted carbocyclyl, substituted or unsubstituted aryl, or substituted or unsubstituted heteroaryl;
R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl;
B is aryl, heterocyclyl, heteroaryl, or carbocyclyl;
each R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, oxo, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —N02, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A} or —S(O)₂R^{A};
n is 1, 2, 3, 4, or 5;
R⁸ is hydrogen, halogen, —CN, or substituted or unsubstituted alkyl;
R¹⁰ is hydrogen, halogen, substituted or unsubstituted alkyl, substituted or unsubstituted carbocyclyl, substituted or unsubstituted aryl, or substituted or unsubstituted heteroaryl; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

### R¹

As described herein, R¹ is hydrogen, substituted or unsubstituted alkyl, or a nitrogen protecting group. In certain embodiments, R¹ is hydrogen or substituted or unsubstituted alkyl. In certain embodiments, R¹ is hydrogen or substituted or unsubstituted C₁₋₄ alkyl. In certain embodiments, R¹ is hydrogen or unsubstituted C₁₋₄ alkyl. In certain embodiments, R¹ is hydrogen or unsubstituted Cia alkyl. In certain embodiments, R¹ is hydrogen or unsubstituted C₁₋₂ alkyl. In certain embodiments, R¹ is hydrogen or methyl. In certain embodiments, R¹ is methyl. In certain embodiments, R¹ is hydrogen. In certain embodiments, R¹ is a nitrogen protecting group (e.g., Bn, Boc, Cbz, Fmoc, trifluoroacetyl, triphenylmethyl, acetyl, or Ts).

### R²

As described herein, R² is hydrogen, halogen, —CN, substituted or unsubstituted alkyl, or substituted or unsubstituted carbocyclyl. In certain embodiments, R² is hydrogen, halogen, —CN, substituted or unsubstituted alkyl, or substituted or unsubstituted cycloalkyl. In certain embodiments, R² is hydrogen, halogen, —CN, or substituted or unsubstituted alkyl. In certain embodiments, R² is hydrogen, halogen, or —CN. In certain embodiments, R² is hydrogen or halogen. In certain embodiments, R² is halogen. In certain embodiments, R² is fluoro, chloro, bromo, or iodo. In certain embodiments, R² is fluoro, chloro, or bromo. In certain embodiments, R² is fluoro or chloro. In certain embodiments, R² is chloro. In certain embodiments, R² is hydrogen or chloro. In certain embodiments, R² is hydrogen or substituted or unsubstituted alkyl. In certain embodiments, R² is hydrogen or substituted or unsubstituted C₁₋₄ alkyl. In certain embodiments, R² is hydrogen or unsubstituted C₁₋₄ alkyl. In certain embodiments, R² is hydrogen.

### R^{2A}

As described herein, R^{2A} is hydrogen, halogen, —CN, or substituted or unsubstituted alkyl. In certain embodiments, R^{2A} is hydrogen, halogen, or —CN. In certain embodiments, R^{2A} is hydrogen or halogen. In certain embodiments, R^{2A} is halogen. In certain embodiments, R^{2A} is fluoro, chloro, bromo, or iodo. In certain embodiments, R^{2A} is fluoro, chloro, or bromo. In certain embodiments, R^{2A} is fluoro or chloro. In certain embodiments, R^{2A} is chloro. In certain embodiments, R^{2A} is hydrogen or chloro. In certain embodiments, R^{2A} is hydrogen or substituted or unsubstituted alkyl. In certain embodiments, R^{2A} is hydrogen or substituted or unsubstituted C₁₋₄ alkyl. In certain embodiments, R^{2A} is hydrogen or unsubstituted C₁₋₄ alkyl. In certain embodiments, R^{2A} is hydrogen.

### R³

As described herein, R³ is hydrogen, substituted or unsubstituted alkyl, or a nitrogen protecting group. In certain embodiments, R³ is hydrogen or substituted or unsubstituted alkyl. In certain embodiments, R³ is hydrogen or substituted or unsubstituted C₁₋₄ alkyl. In certain embodiments, R³ is hydrogen or unsubstituted C₁₋₄ alkyl. In certain embodiments, R³ is hydrogen. In certain embodiments, R³ is a nitrogen protecting group (e.g., Bn, Boc, Cbz, Fmoc, trifluoroacetyl, triphenylmethyl, acetyl, or Ts).

### X, Y, and Z

As described herein, X is CR⁷ or N; Y is CR⁸ or N; and Z is CR⁹ or N; wherein each R⁷, R⁹, and R⁹ is independently hydrogen, halogen, —CN, or substituted or unsubstituted alkyl.

In certain embodiments, X is CR⁷. In certain embodiments, X is N. In certain embodiments, X is CR⁷; and R⁷ is hydrogen, halogen, or —CN. In certain embodiments, X is CR⁷; and R⁷ is hydrogen or halogen, preferably hydrogen. In certain embodiments, X is CR⁷; and R⁷ is halogen. In certain embodiments, X is CR⁷; and R⁷ is fluoro, chloro, bromo, or iodo. In certain embodiments, X is CR⁷; and R⁷ is fluoro, chloro, or bromo. In certain embodiments, X is CR⁷; and R⁷ is fluoro or chloro. In certain embodiments, X is CR⁷; and R⁷ is chloro. In certain embodiments, X is CR⁷; and R⁷ is hydrogen or chloro. In certain embodiments, X is CR⁷; and R⁷ is hydrogen. In certain embodiments, X is CR⁷; and R⁷ is substituted or unsubstituted alkyl. In certain embodiments, X is CR⁷; and R⁷ is substituted or unsubstituted C₁₋₄ alkyl. In certain embodiments, X is CR⁷; and R⁷ is unsubstituted C₁₋₄ alkyl. In certain embodiments, X is CR⁷; and R⁷ is isopropyl.

In certain embodiments, Y is CR⁸. In certain embodiments, Y is N. In certain embodiments, Y is CR⁸; and R⁹ is hydrogen, halogen, or —CN. In certain embodiments, Y is CR⁸; and R⁹ is hydrogen or halogen. In certain embodiments, Y is CR⁸; and R⁹ is hydrogen. In certain embodiments, Z is CR⁹. In certain embodiments, Z is N. In certain embodiments, Z is CR⁹; and R⁹ is hydrogen, halogen, or —CN. In certain embodiments, Z is CR⁹; and R⁹ is hydrogen or halogen. In certain embodiments, Z is CR⁹; and R⁹ is hydrogen. In certain embodiments, X is CR⁷, wherein R⁷ is hydrogen or halogen, preferably hydrogen; Y is CR⁸, wherein R⁹ is hydrogen; and Z is CR⁹, wherein R⁹ is hydrogen.

### B and R⁶

As described herein, B is aryl, heterocyclyl, heteroaryl, or carbocyclyl. In certain embodiments, B is aryl, heterocyclyl, or heteroaryl. In certain embodiments, B is aryl or heteroaryl. In certain embodiments, B is monocyclic aryl or monocyclic heteroaryl. In certain embodiments, B is 6-membered aryl or 5- or 6-membered heteroaryl. In certain embodiments, B is phenyl or pyridinyl. In certain embodiments, B is phenyl. In certain embodiments, B is pyridinyl. In certain embodiments, B is 2-pyridinyl. In certain embodiments, B is 3-pyridinyl. In certain embodiments, B is 4-pyridinyl. In certain embodiments, B is carbocyclyl (e.g., monocyclic carbocyclyl). In certain embodiments, B is cyclopropyl, cyclobutyl, cyclopentyl, cyclohexyl, or cycloheptyl.

In certain embodiments, n is 1, 2, 3, 4, or 5. In certain embodiments, n is 1, 2, 3, or 4. In certain embodiments, n is 1, 2, or 3. In certain embodiments, n is 1 or 2. In certain embodiments, n is 1. In certain embodiments, n is 2. In certain embodiments, n is 3. In certain embodiments, n is 4. In certain embodiments, n is 5.

In certain embodiments,

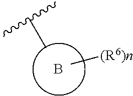

### (—B(R) n) is

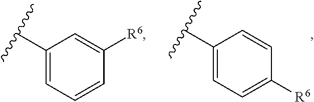

or

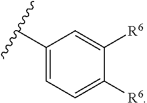

In certain embodiments, —B(R⁶)ₙ is

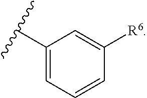

In certain embodiments, —B(R⁶)ₙ is

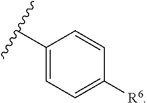

In certain embodiments, —B(R⁶)ₙ is

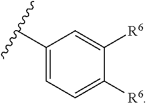

In certain embodiments, —B(R⁶)ₙ is

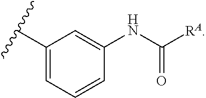

In certain embodiments, —B(R⁶)ₙ is

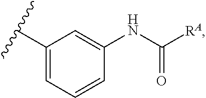

wherein R^{A} is a 5-6 membered substituted or unsubstituted heteroaryl.

In certain embodiments, —B(R⁶)ₙ is

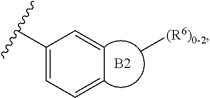

wherein B2 is 5-6-membered, monocyclic, unsubstituted heteroaryl, or 5-6-membered, monocyclic, unsubstituted heterocyclyl, and R⁶ is directly attached to B2. In certain embodiments, —B(R⁶)ₙ is

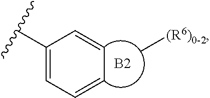

wherein B2 is 5-6-membered, monocyclic, unsubstituted heterocyclyl, and R⁶ is directly attached to B2. In certain embodiments, —B(R⁶)ₙ is

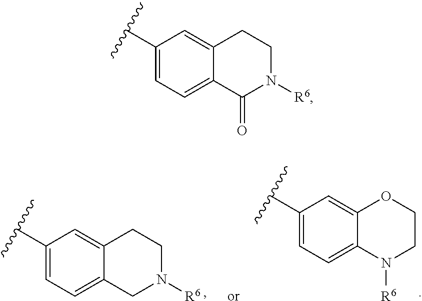

In certain embodiments, —B(R⁶)ₙ

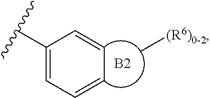

wherein B2 is 5-6-membered, monocyclic, unsubstituted heteroaryl, and R⁶ is directly attached to B2. In certain embodiments, —B(R⁶)ₙ is

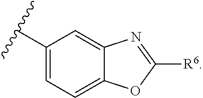

In certain embodiments, —B(R⁶)ₙ is

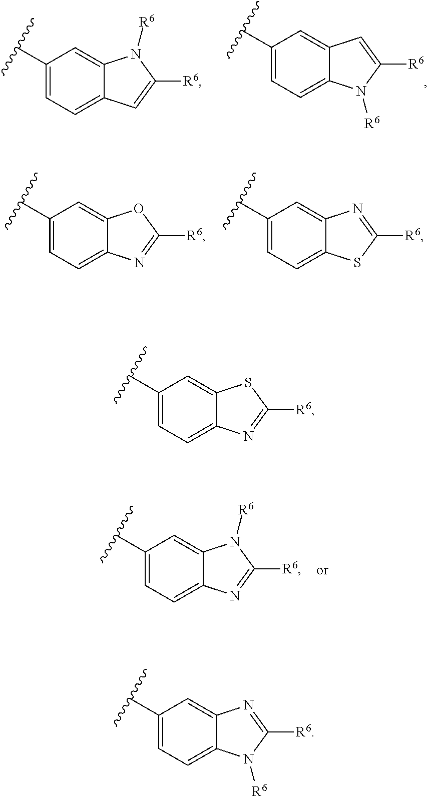

In certain embodiments, —B(R⁶)ₙ is

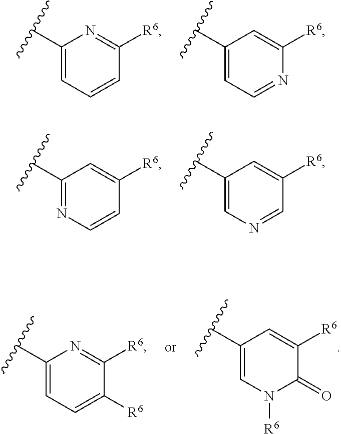

In certain embodiments, —B(R⁶)ₙ is

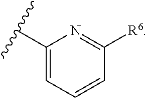

In certain embodiments, —B(R⁶)ₙ is

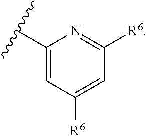

In certain embodiments, —B(R⁶)ₙ is

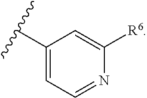

In certain embodiments, —B(R⁶)ₙ is

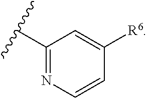

In certain embodiments, —B(R⁶)

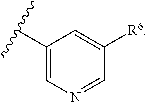

In certain embodiments, —B(R⁶)ₙ is

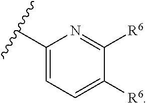

In certain embodiments, —B(R⁶)ₙ is

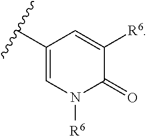

In certain embodiments, —B(R⁶)ₙ is

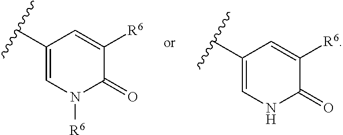

In certain embodiments, —B(R⁶)ₙ is

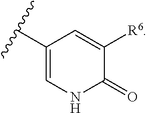

As described herein, each R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, oxo, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —N02, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}.

In certain embodiments, each R⁶ is independently halogen, substituted or unsubstituted alkyl, substituted or unsubstituted aryl, substituted or unsubstituted heteroaryl, substituted or unsubstituted heteroaliphatic, oxo, —OR^{A}, —N(R^{A})₂, —CN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}.

In certain embodiments, each R⁶ is independently halogen, substituted or unsubstituted alkyl, substituted or unsubstituted heteroaryl, oxo, —C(═O)R^{A}, —C(═O)N(R^{A})₂, —S(O)₂R^{A}, —OR^{A}, —N(R^{A})₂, or —NR^{A}C(═O)R^{A}. In certain embodiments, each R⁶ is independently substituted or unsubstituted alkyl, substituted or unsubstituted heteroaryl, oxo, —OR^{A}, —N(R^{A})₂, or —NR^{A}C(═O)R^{A}. In certain embodiments, each R⁶ is independently substituted or unsubstituted alkyl, substituted or unsubstituted heteroaryl, oxo, —OC₁₋₄ alkyl, —OCH₂C(═O)N(R^{A})₂, —N(R^{A})₂, or —NR^{A}C(═O)R^{A}. In certain embodiments, each R⁶ is independently C₁₄ alkyl, substituted or unsubstituted heteroaryl, oxo, —OC₁₋₄ alkyl, —OCH₂C(═O)NHC₁₋₄ alkyl, —NH₂, —NHC(═O)aryl, or —NHC(═O)heteroaryl, wherein each alkyl, aryl, and heteroaryl are substituted or unsubstituted.

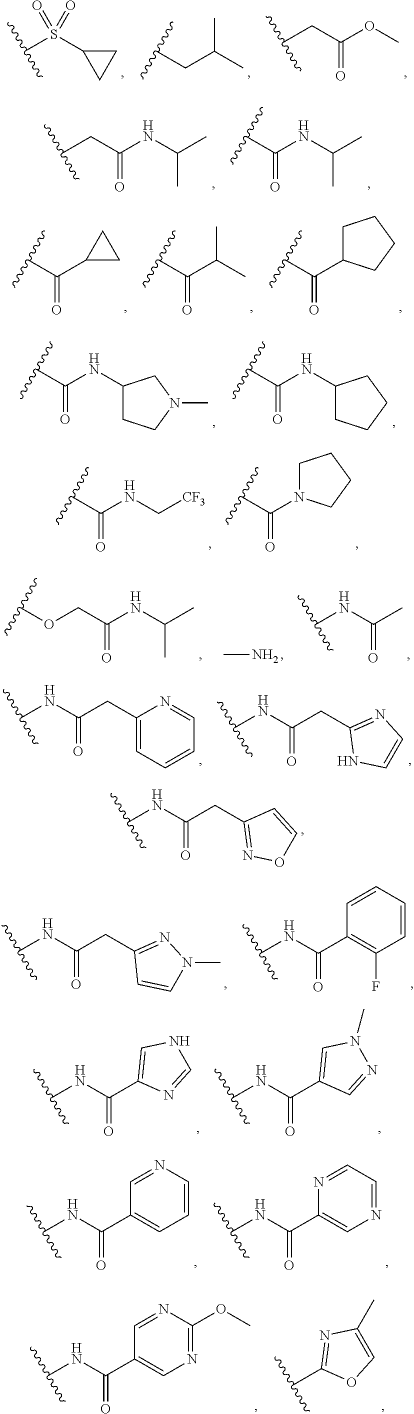

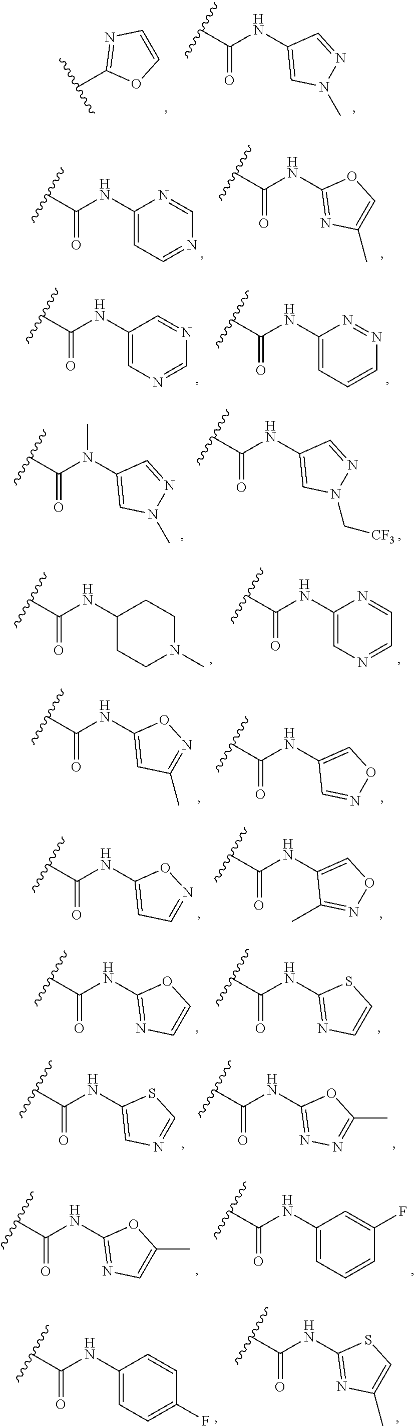

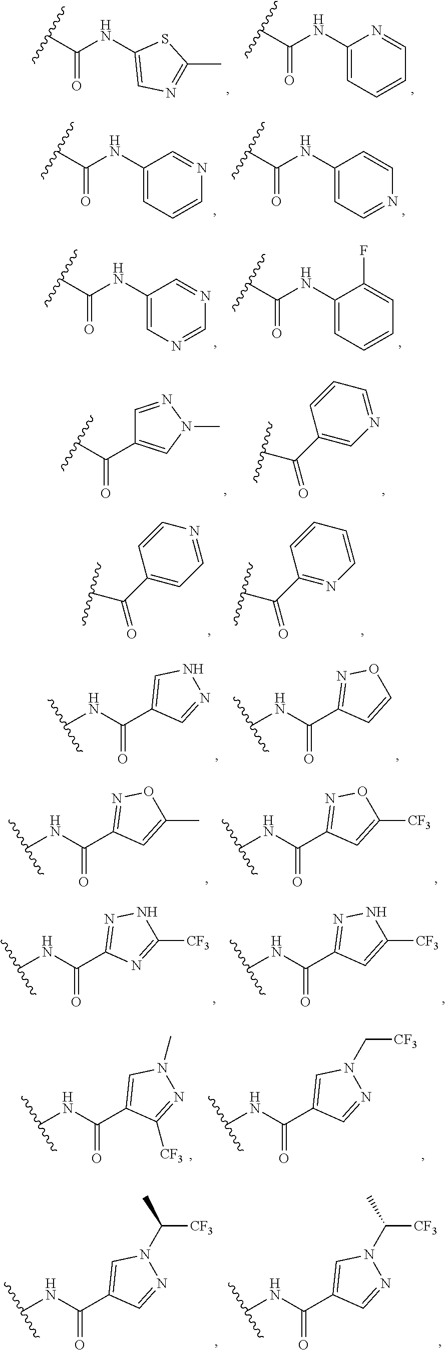

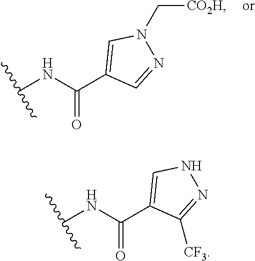 [SMILES: *NC(=O)c1cnn(CC(=O)O)c1 ; *NC(=O)c1c[nH]nc1C(F)(F)F]

In certain embodiments, each R⁶ is independently oxo, —CH₃, —OCH₃, —F,

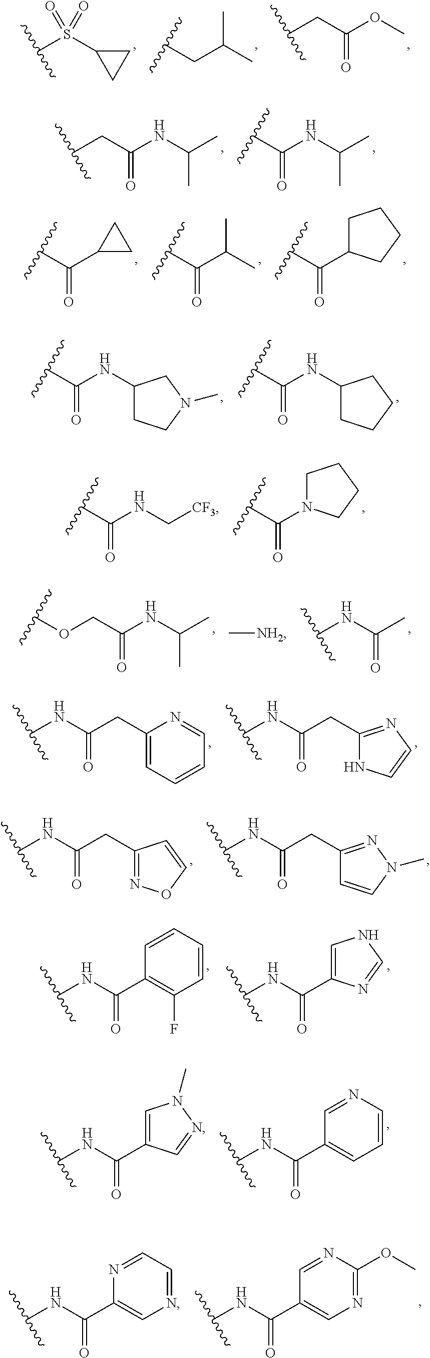

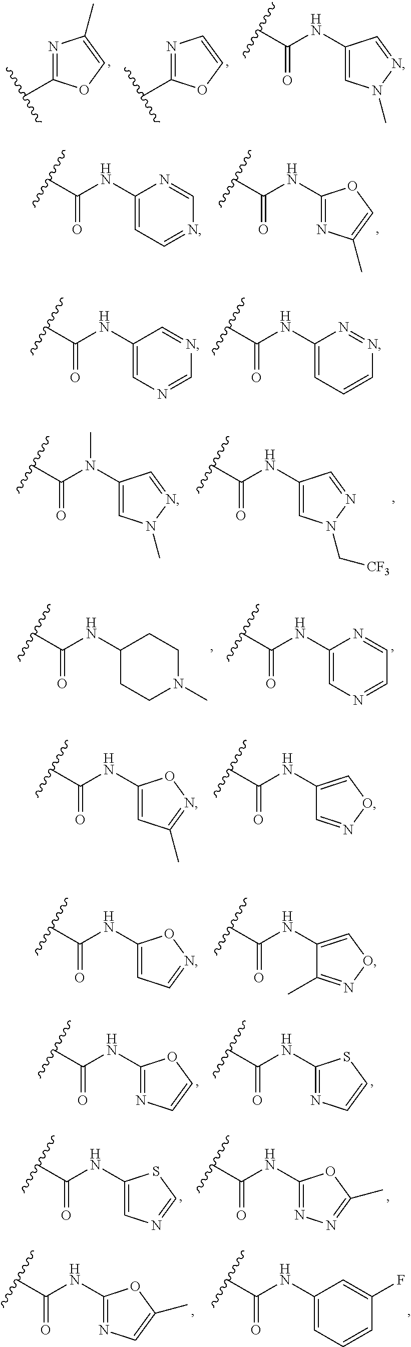

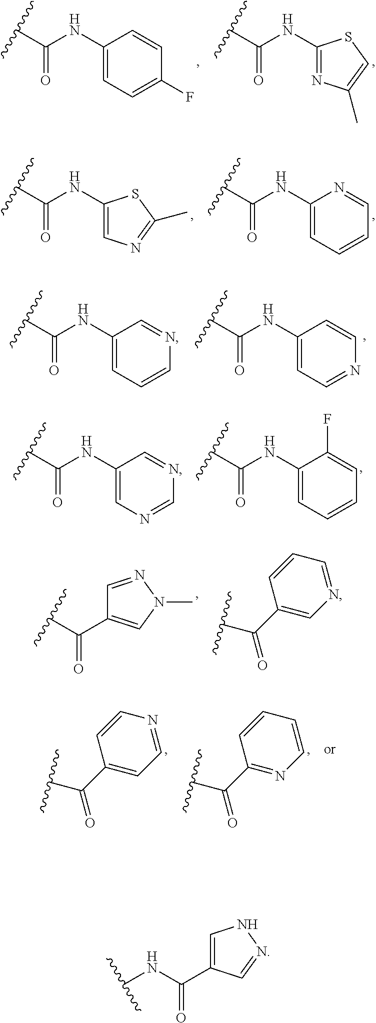

In certain embodiments, each R⁶ is independently oxo, —CH₃, —OCH₃, —F,

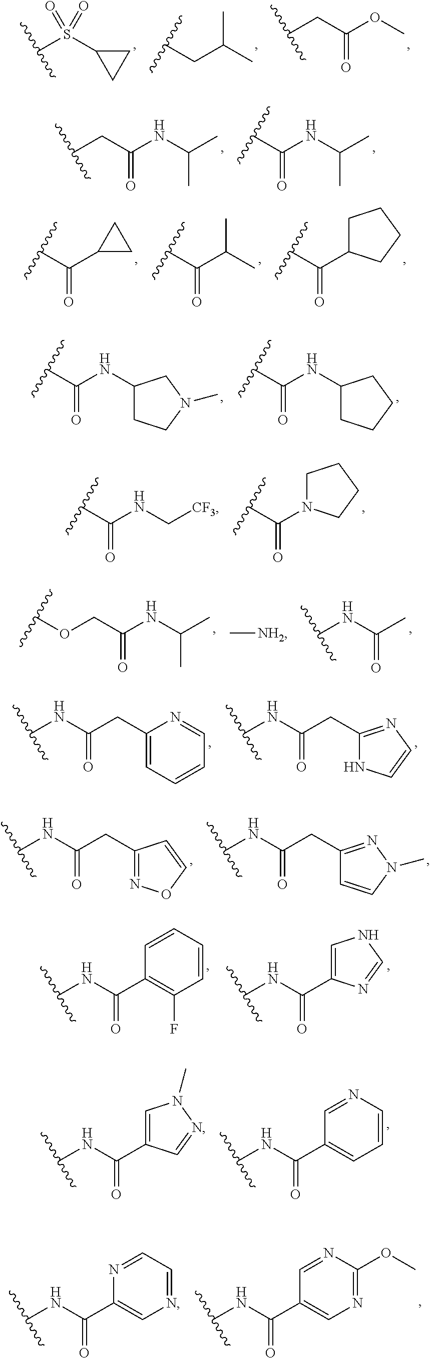

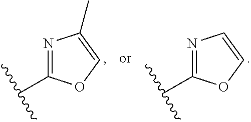

In certain embodiments, each R⁶ is independently oxo, —CH₃, —OCH₃,

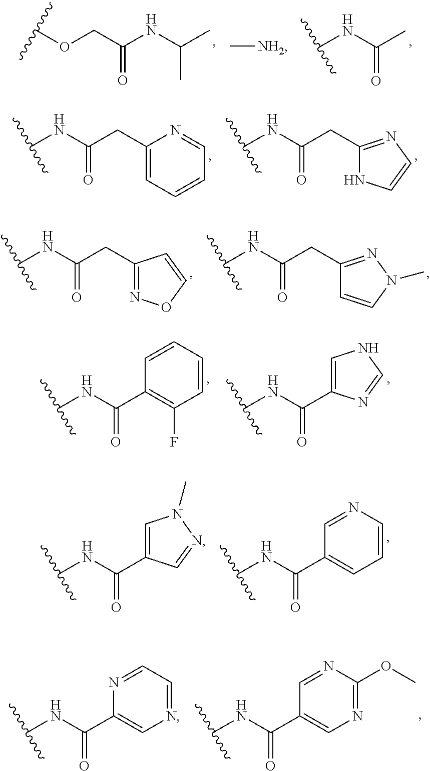

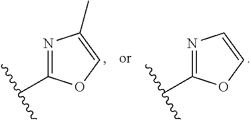

In certain embodiments, each R⁶ is independently —OCH₃,

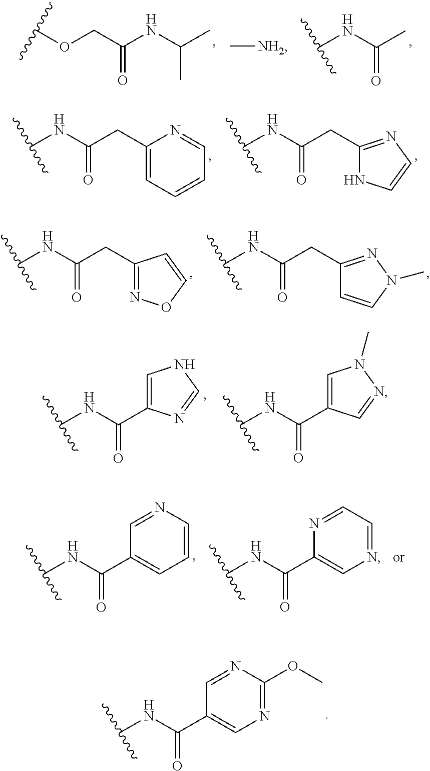

In certain embodiments, R⁶ is

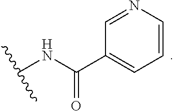 [SMILES: *NC(=O)c1cccnc1]

In certain embodiments, R⁶ is

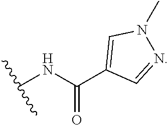 [SMILES: *NC(=O)c1cnn(C)c1]

In certain embodiments —B(R⁶)ₙ is

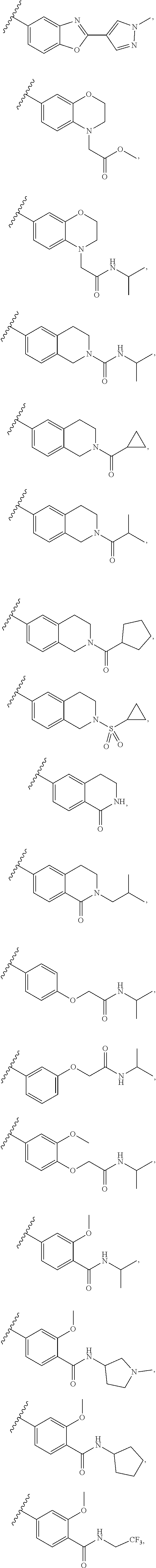

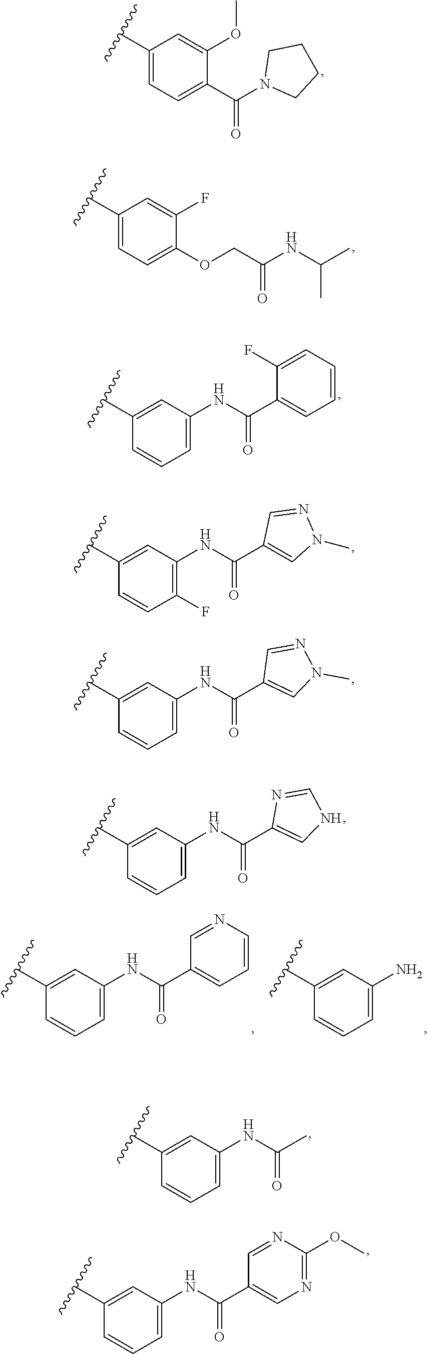

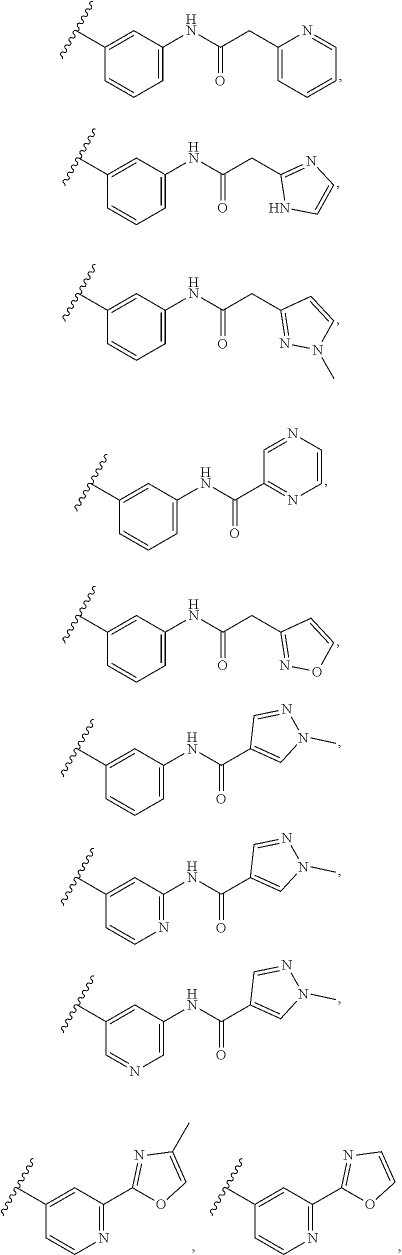

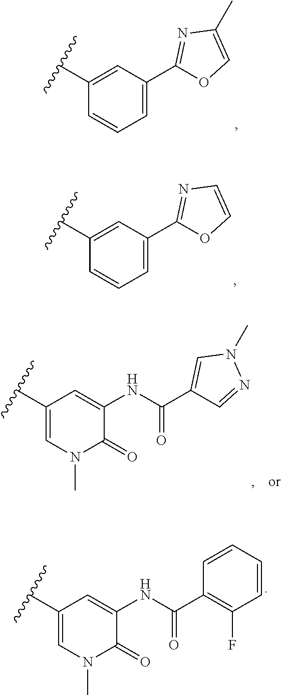 [SMILES: *c1cccc(-c2nc(C)co2)c1 ; *c1cccc(-c2ncco2)c1 ; *c1cc(NC(=O)c2cnn(C)c2)c(=O)n(C)c1 ; *c1cc(NC(=O)c2ccccc2F)c(=O)n(C)c1]

In certain embodiments, —B(R⁶)ₙ is

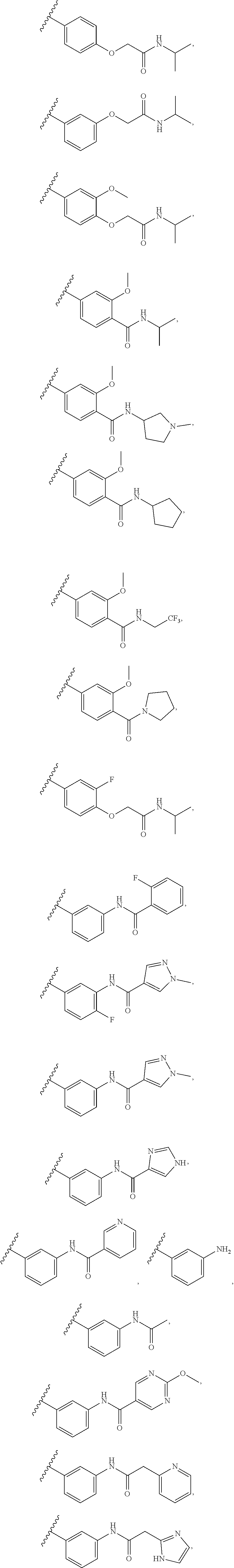

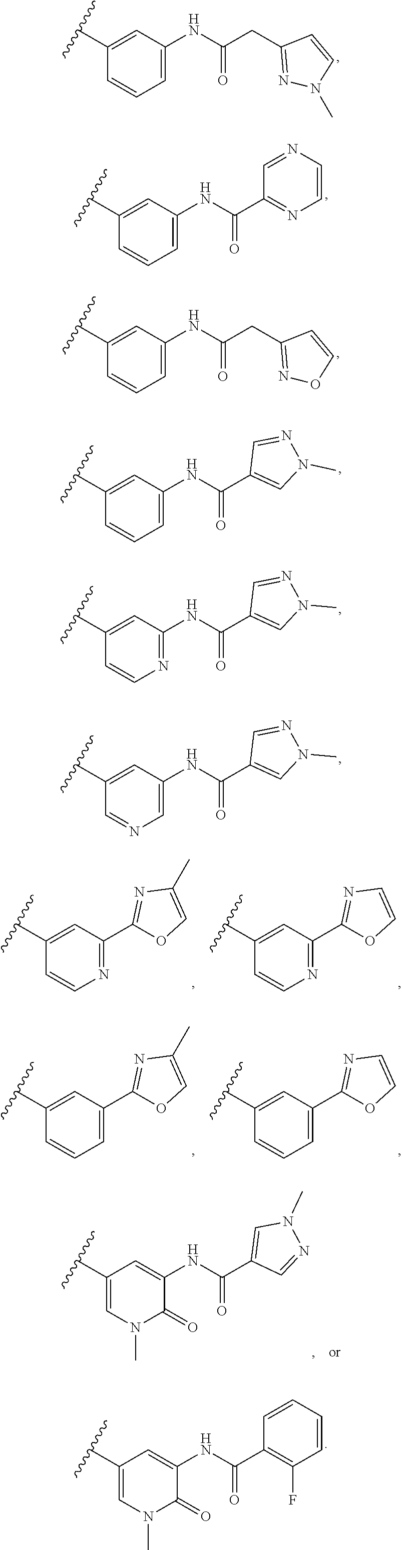

In certain embodiments, —B(R⁶)ₙ is

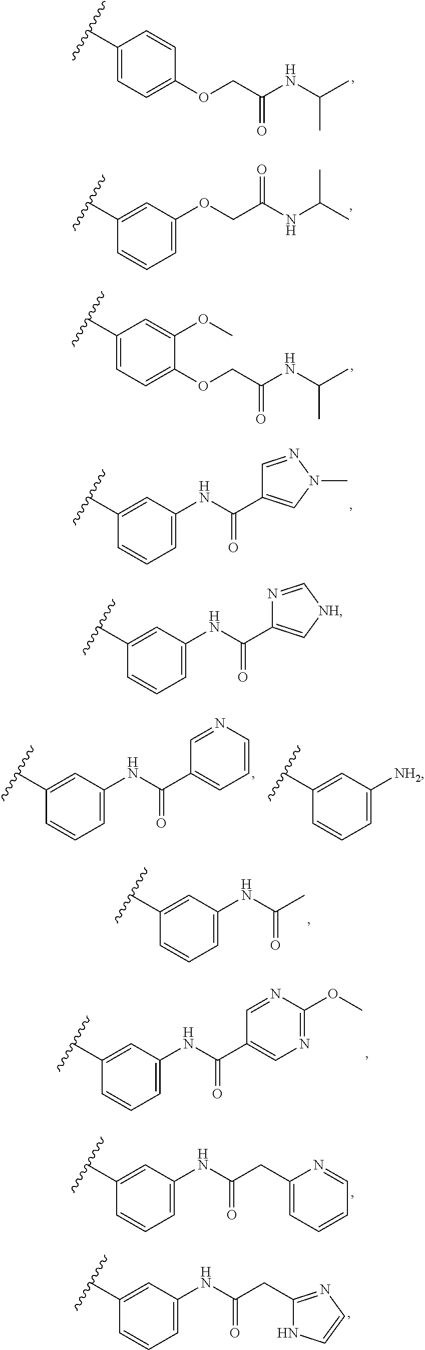

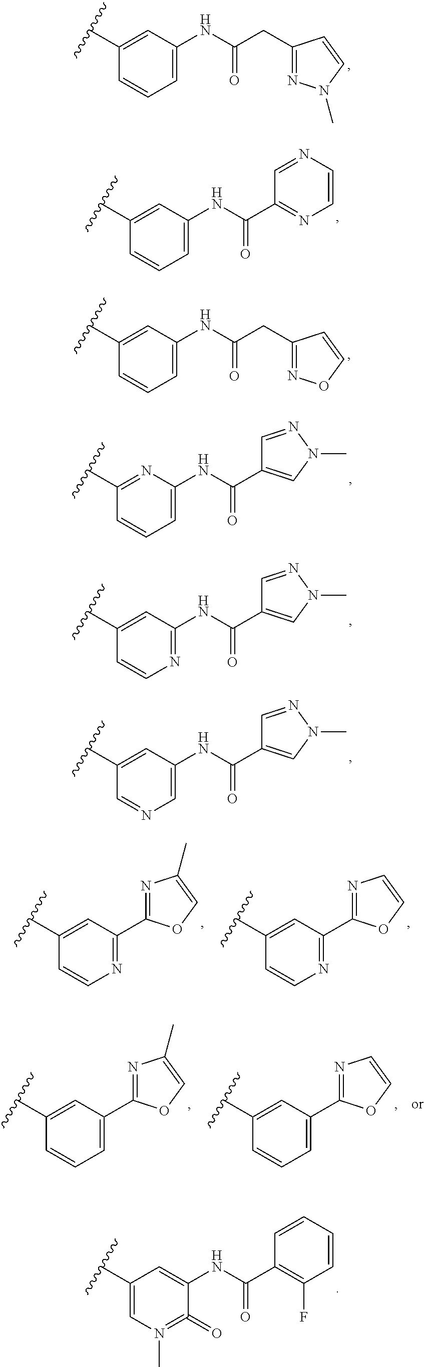

In certain embodiments, —B(R⁶)ₙ is

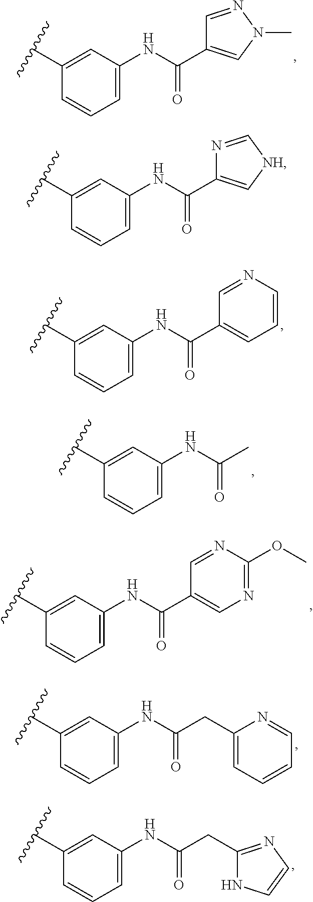

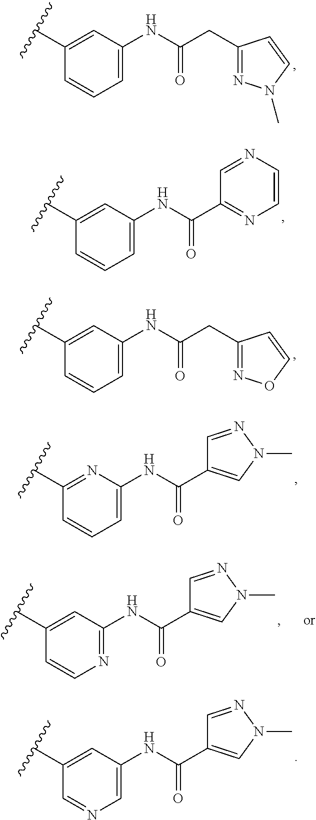

In certain embodiments, —B(R⁶)ₙ is

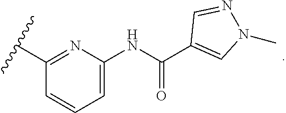 [SMILES: *c1cccc(NC(=O)c2cnn(C)c2)n1]

In certain embodiments, —B(R⁶)ₙ is

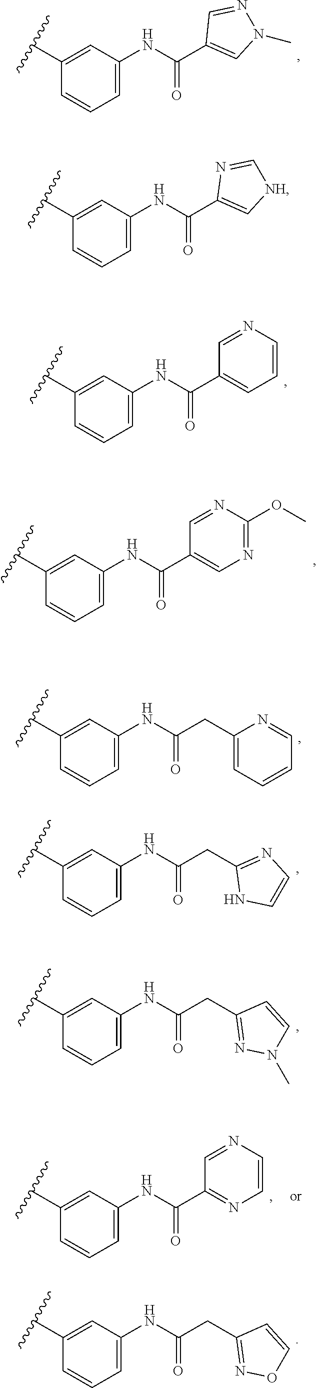

In certain embodiments, —B(R⁶)ₙ is

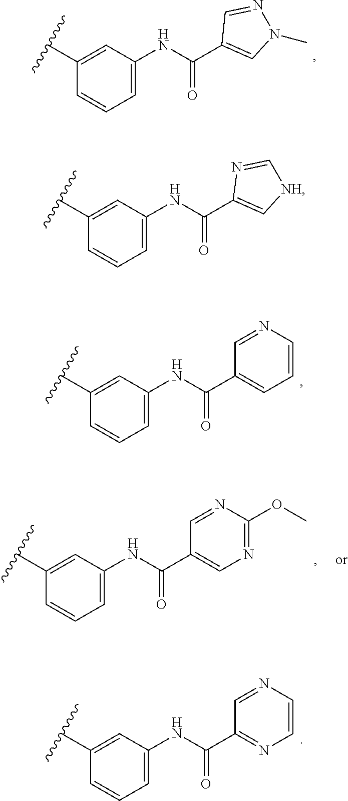 [SMILES: *c1cccc(NC(=O)c2cnn(C)c2)c1 ; *c1cccc(NC(=O)c2c[nH]cn2)c1 ; *c1cccc(NC(=O)c2cccnc2)c1 ; *c1cccc(NC(=O)c2cnc(OC)nc2)c1 ; *c1cccc(NC(=O)c2cnccn2)c1]

In certain embodiments, —B(R)ₙ is

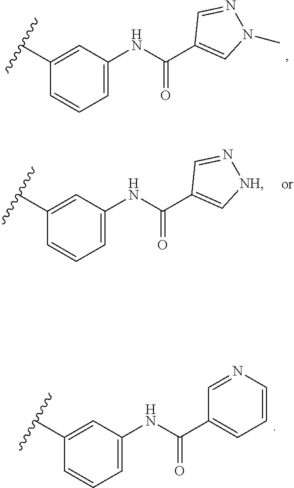 [SMILES: *c1cccc(NC(=O)c2cnn(C)c2)c1 ; *c1cccc(NC(=O)c2cn[nH]c2)c1 ; *c1cccc(NC(=O)c2cccnc2)c1]

In certain embodiments, —B(R⁶)ₙ is

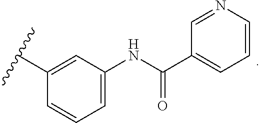 [SMILES: *c1cccc(NC(=O)c2cccnc2)c1]

In certain embodiments, —B(R⁶)ₙ is

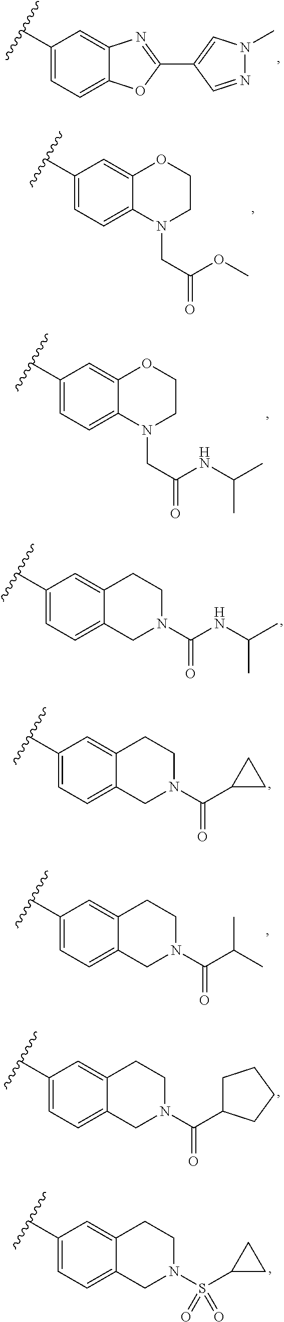

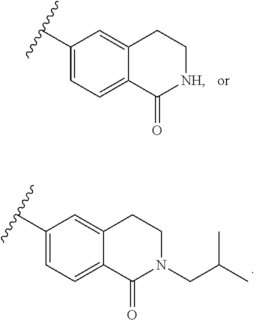 [SMILES: *c1ccc2c(c1)CCNC2=O ; *c1ccc2c(c1)CCN(CC(C)C)C2=O]

In certain embodiments, —B(R⁶)ₙ is

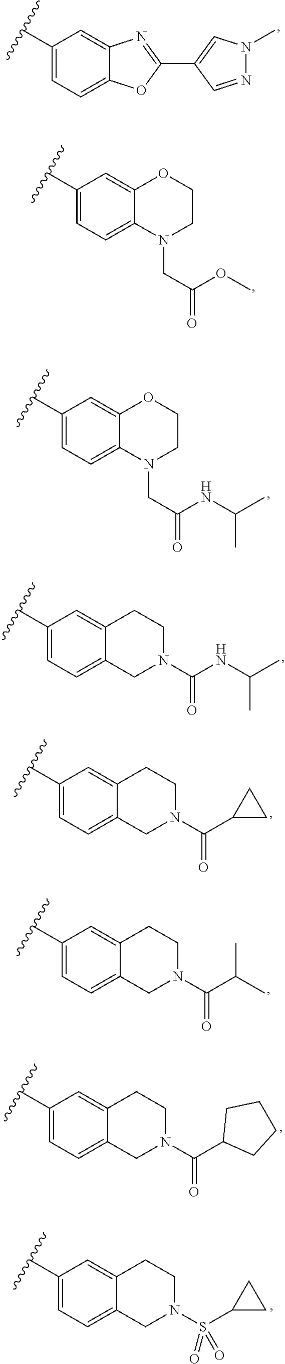

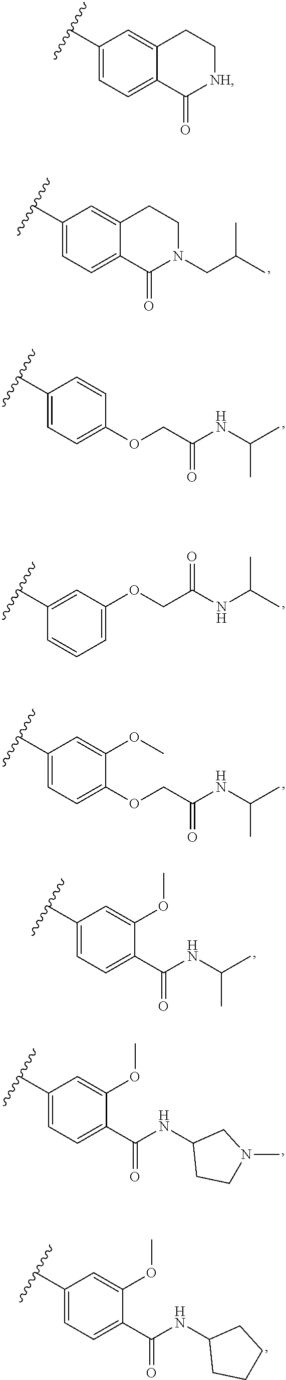

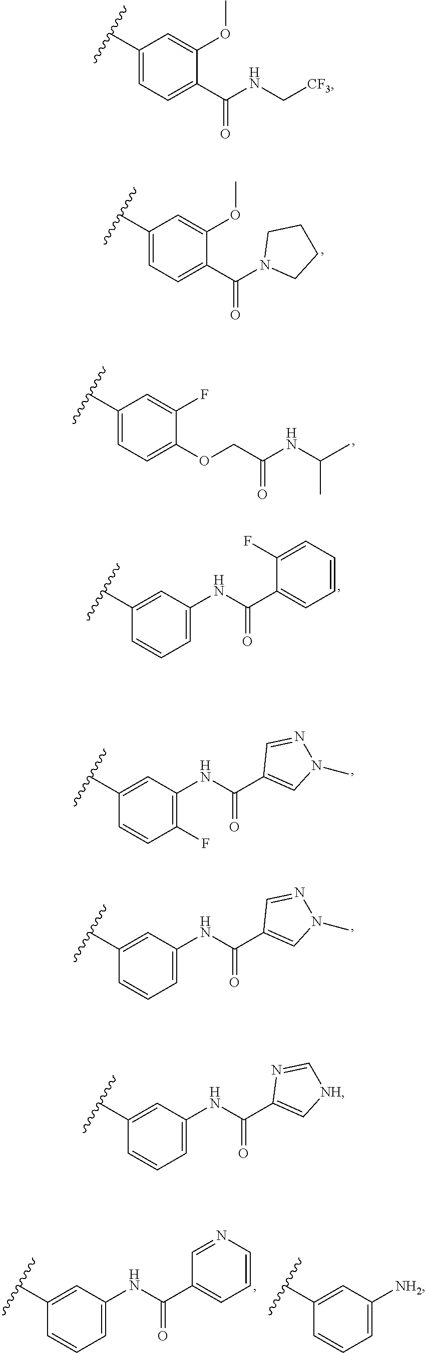

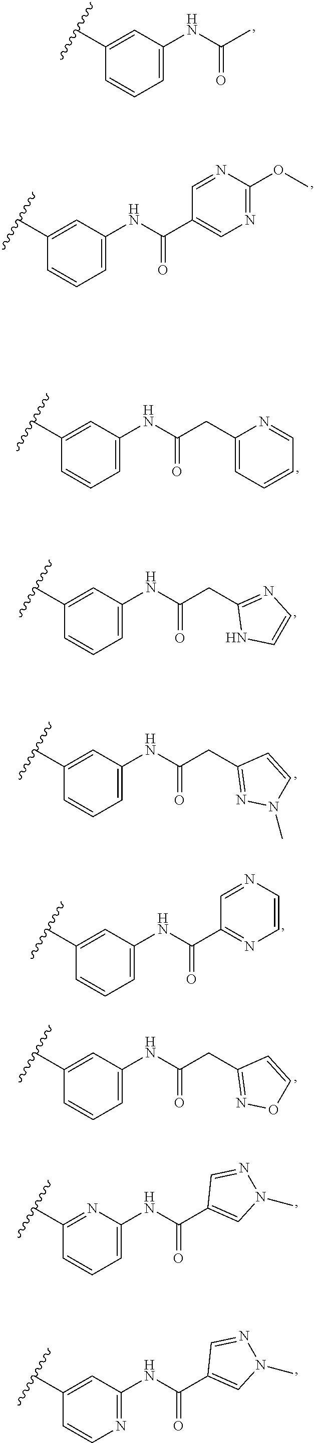

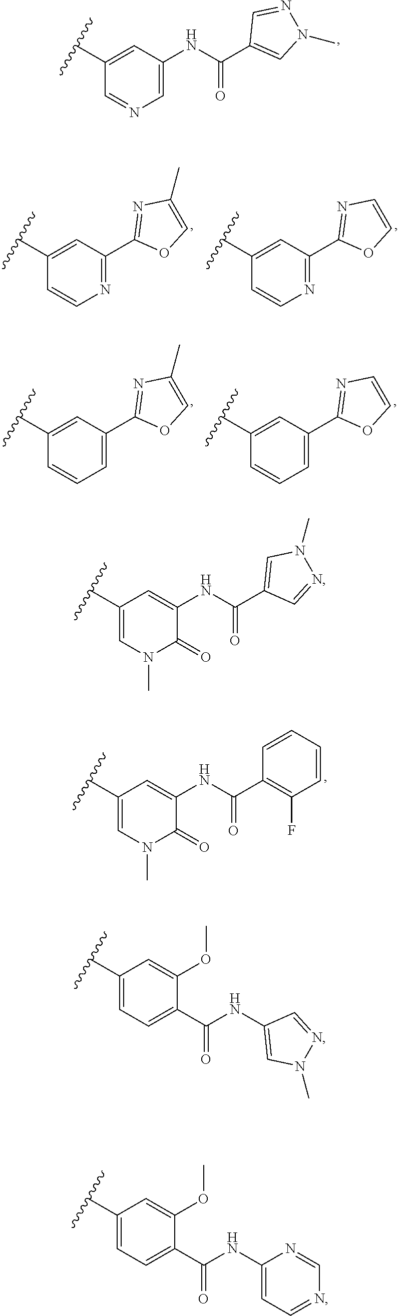

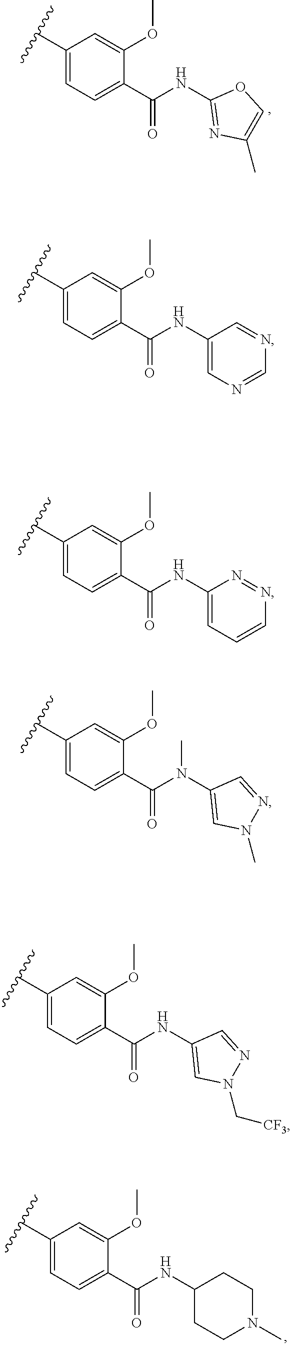 [SMILES: *c1ccc(C(=O)Nc2nc(C)co2)c(OC)c1 ; *c1ccc(C(=O)Nc2cncnc2)c(OC)c1 ; *c1ccc(C(=O)Nc2cccnn2)c(OC)c1 ; *c1ccc(C(=O)N(C)c2cnn(C)c2)c(OC)c1 ; *c1ccc(C(=O)Nc2cnn(CC(F)(F)F)c2)c(OC)c1 ; *c1ccc(C(=O)NC2CCN(C)CC2)c(OC)c1]

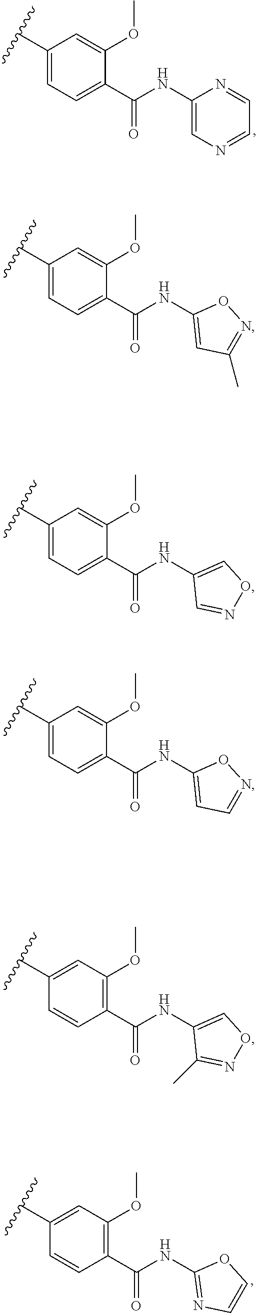 [SMILES: *c1ccc(C(=O)Nc2cnccn2)c(OC)c1 ; *c1ccc(C(=O)Nc2cc(C)no2)c(OC)c1 ; *c1ccc(C(=O)Nc2cnoc2)c(OC)c1 ; *c1ccc(C(=O)Nc2ccno2)c(OC)c1 ; *c1ccc(C(=O)Nc2conc2C)c(OC)c1 ; *c1ccc(C(=O)Nc2ncco2)c(OC)c1]

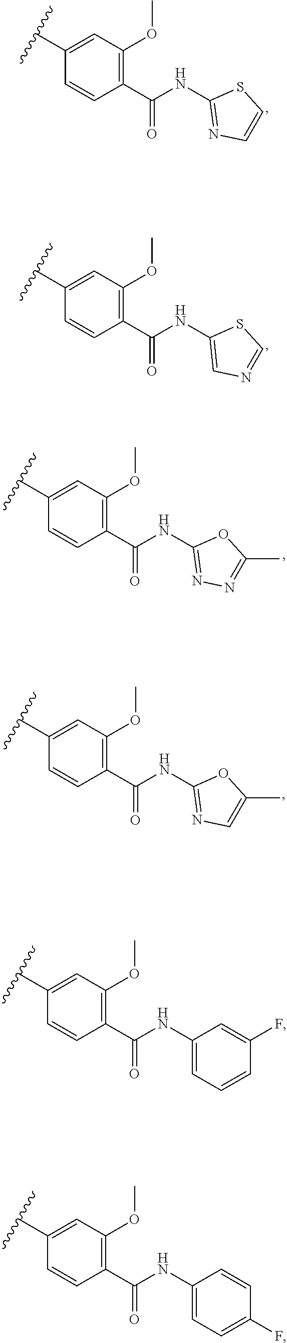 [SMILES: *c1ccc(C(=O)Nc2nccs2)c(OC)c1 ; *c1ccc(C(=O)Nc2cncs2)c(OC)c1 ; *c1ccc(C(=O)Nc2nnc(C)o2)c(OC)c1 ; *c1ccc(C(=O)Nc2ncc(C)o2)c(OC)c1 ; *c1ccc(C(=O)Nc2cccc(F)c2)c(OC)c1 ; *c1ccc(C(=O)Nc2ccc(F)cc2)c(OC)c1]

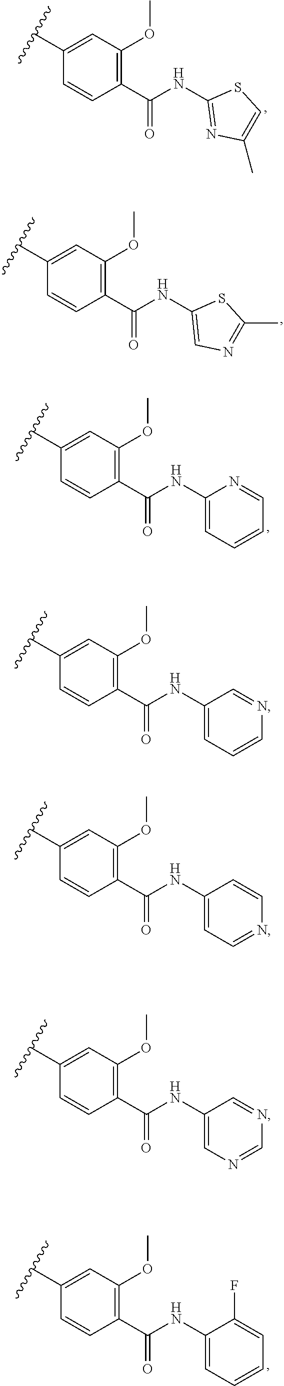

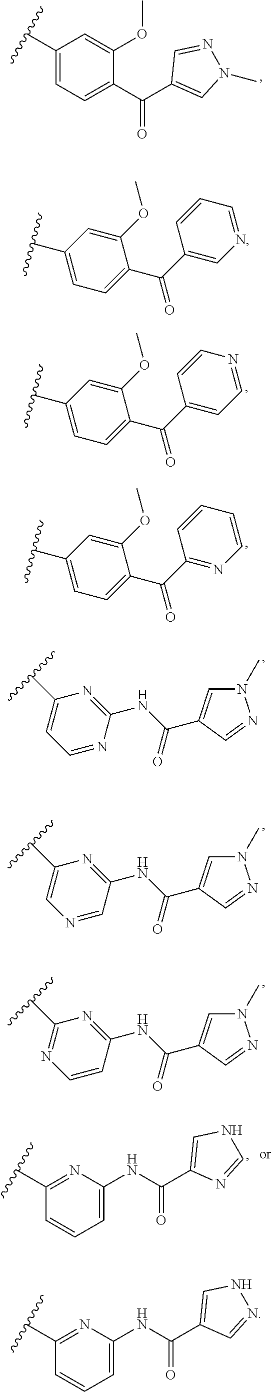

In certain embodiments —B(R⁶)ₙ is

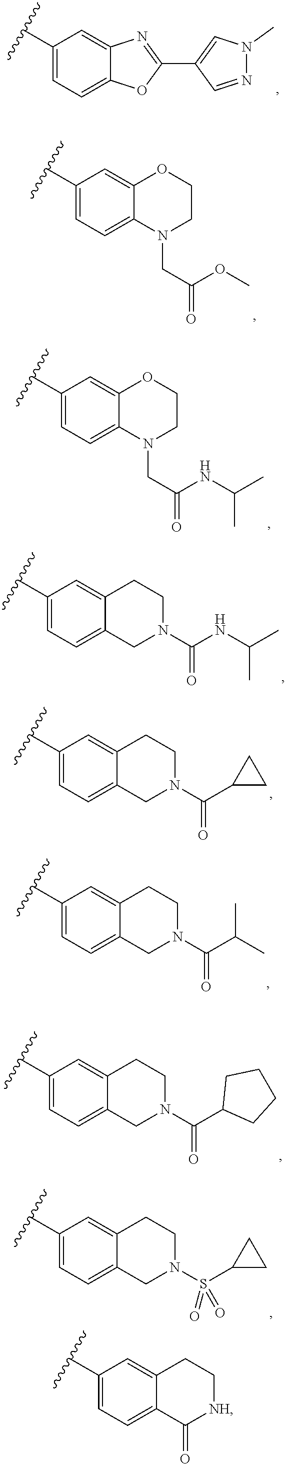

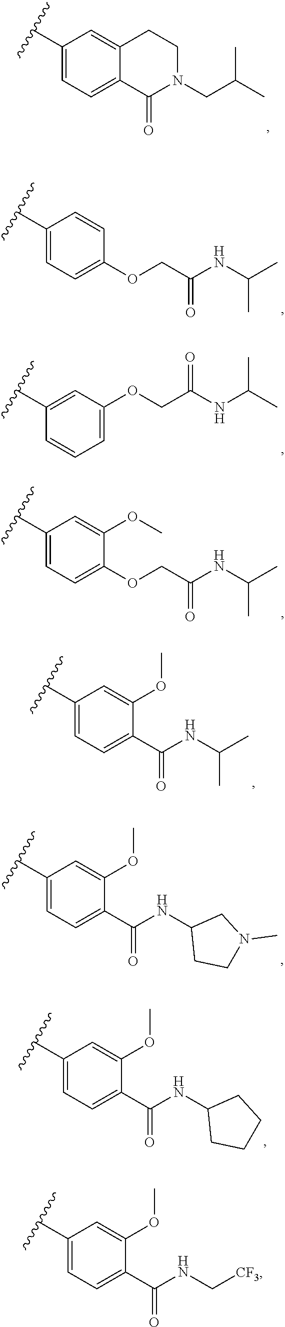

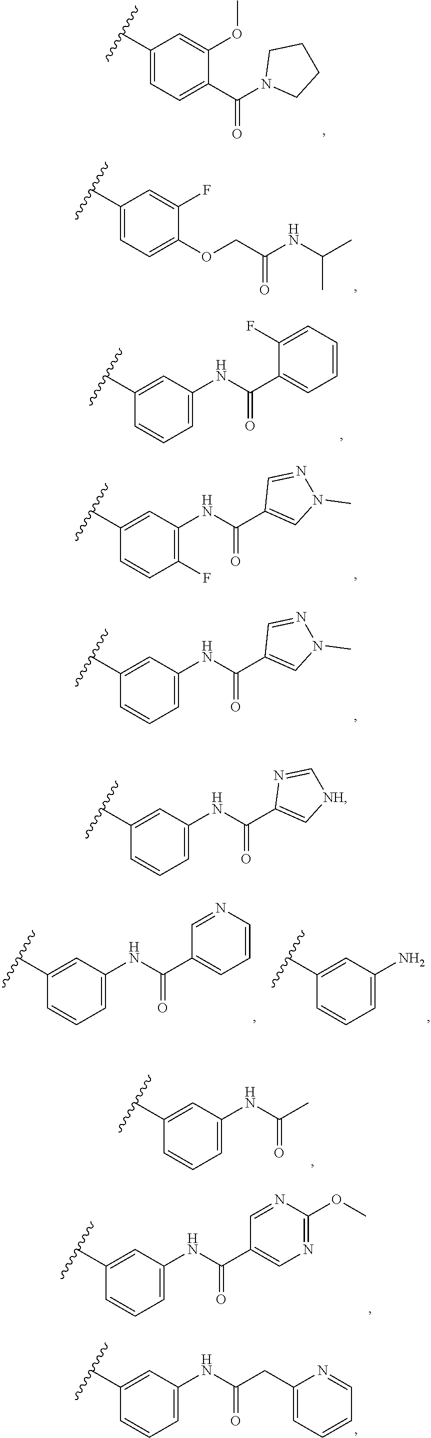

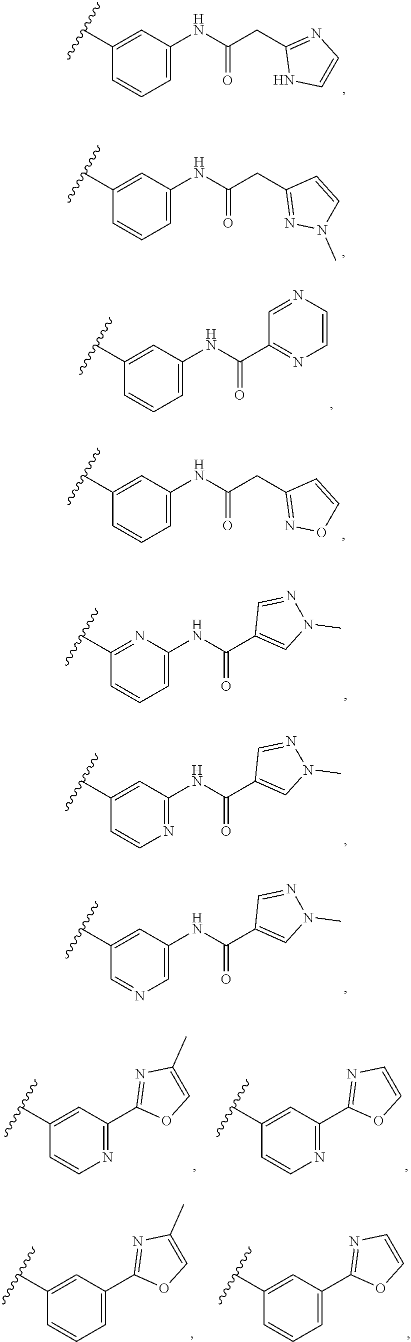

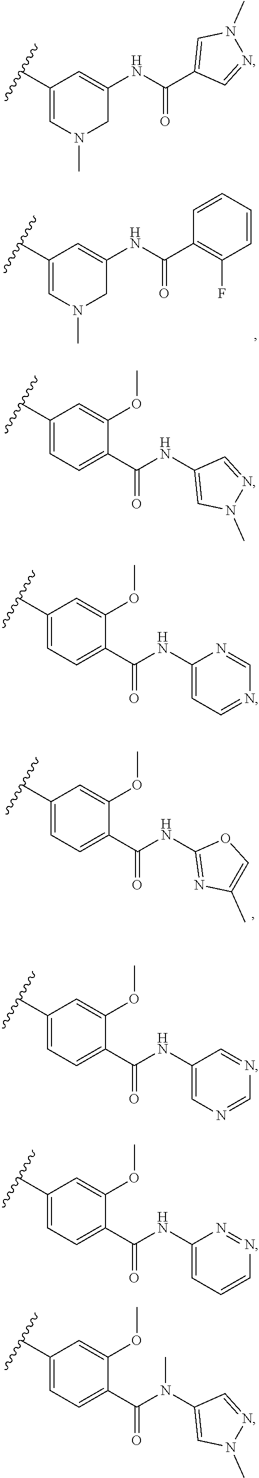

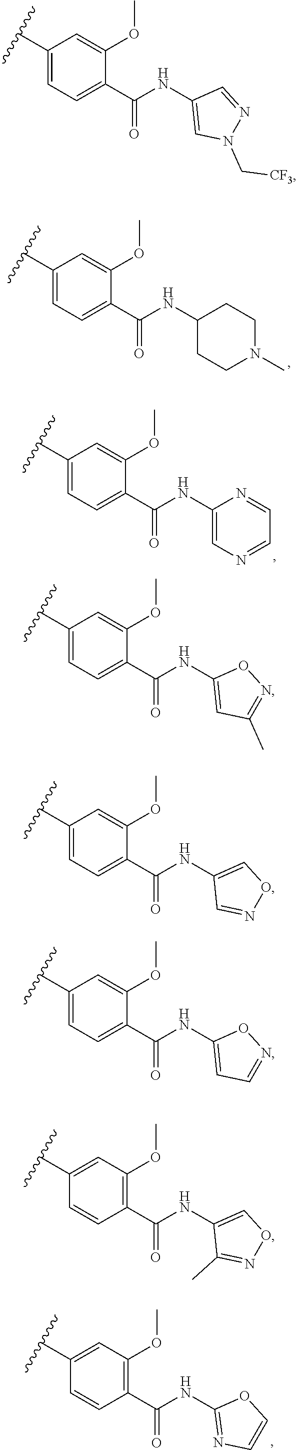

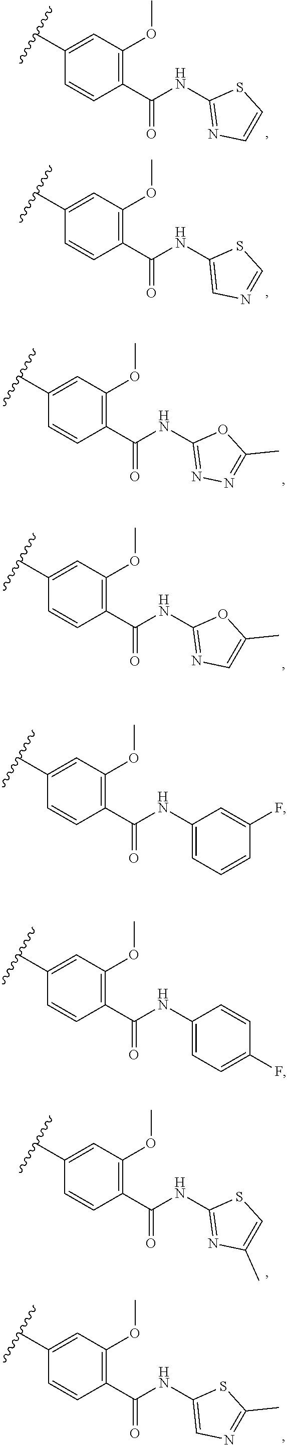

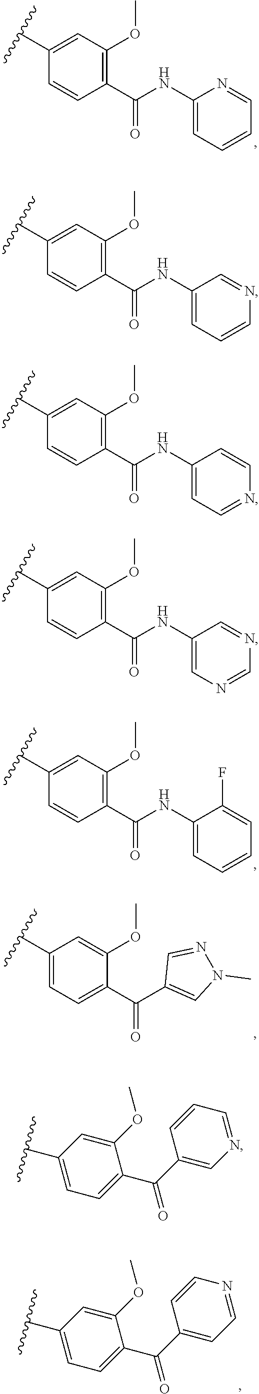

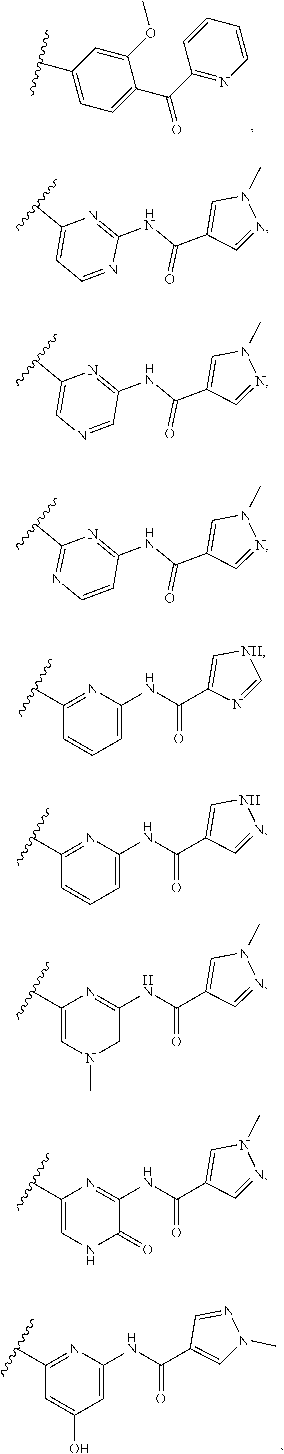

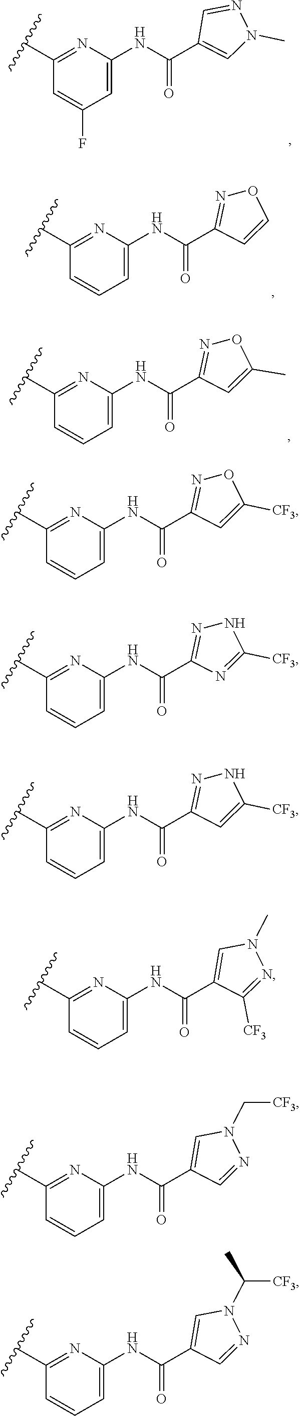

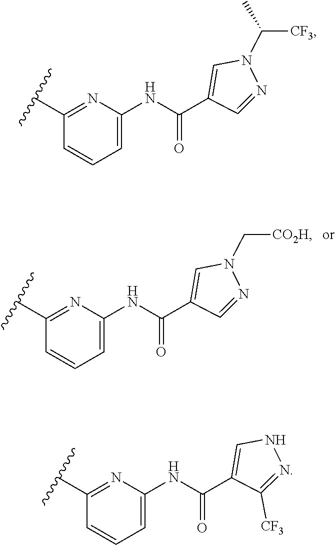 [SMILES: *c1cccc(NC(=O)c2cnn([C@H](C)C(F)(F)F)c2)n1 ; *c1cccc(NC(=O)c2cnn(CC(=O)O)c2)n1 ; "or" ; *c1cccc(NC(=O)c2c[nH]nc2C(F)(F)F)n1]

### Ring A

As described herein, A is

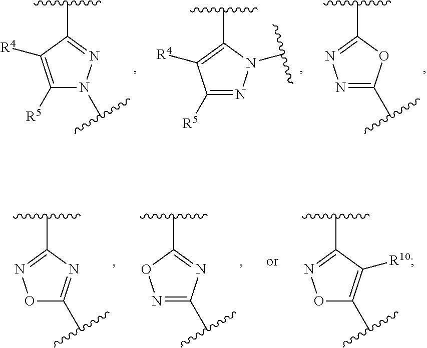

wherein R⁴ is hydrogen, halogen, substituted or unsubstituted alkyl, substituted or unsubstituted carbocyclyl, substituted or unsubstituted aryl, or substituted or unsubstituted heteroaryl; R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl; and R¹⁰ is hydrogen, halogen, substituted or unsubstituted alkyl, substituted or unsubstituted carbocyclyl, substituted or unsubstituted aryl, or substituted or unsubstituted heteroaryl.

In certain embodiments, A is

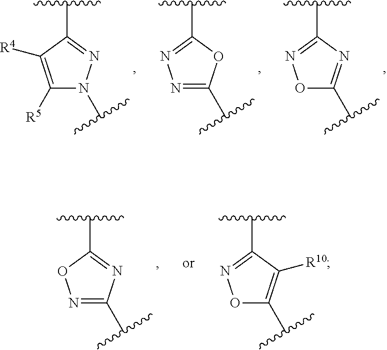

wherein R⁴ is hydrogen, halogen, substituted or unsubstituted alkyl, substituted or unsubstituted carbocyclyl, substituted or unsubstituted aryl, or substituted or unsubstituted heteroaryl; R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl; and R¹⁰ is hydrogen, halogen, substituted or unsubstituted alkyl, substituted or unsubstituted carbocyclyl, substituted or unsubstituted aryl, or substituted or unsubstituted heteroaryl.

In certain embodiments, A is

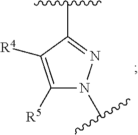

wherein R⁴ is hydrogen, halogen, substituted or unsubstituted alkyl, substituted or unsubstituted carbocyclyl, substituted or unsubstituted aryl, or substituted or unsubstituted heteroaryl; and R⁵ is hydrogen, halogen, substituted or unsubstituted alkyl, substituted or unsubstituted carbocyclyl, substituted or unsubstituted aryl, or substituted or unsubstituted heteroaryl. In certain embodiments, R⁴ is hydrogen or substituted or unsubstituted alkyl. In certain embodiments, R⁴ is hydrogen. In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ haloalkyl. In certain embodiments, R⁴ is methyl or trifluoromethyl. In certain embodiments, R⁴ is methyl. In certain embodiments, R⁴ is trifluoromethyl. In certain embodiments, R⁴ is substituted or unsubstituted carbocyclyl (e.g., substituted or unsubstituted, monocyclic carbocyclyl). In certain embodiments, R⁴ is unsubstituted, cyclopropyl, cyclobutyl, cyclopentyl, cyclohexyl, or cycloheptyl. In certain embodiments, R⁴ is substituted cyclopropyl, cyclobutyl, cyclopentyl, cyclohexyl, or cycloheptyl (e.g., substituted with halogen, unsubstituted alkyl, and/or —O-(unsubstituted alkyl)). In certain embodiments, R⁴ is substituted or unsubstituted aryl. In certain embodiments, R⁴ is unsubstituted phenyl. In certain embodiments, R⁴ is substituted phenyl (e.g., substituted with halogen, unsubstituted alkyl, and/or —O-(unsubstituted alkyl)). In certain embodiments, R⁴ is substituted or unsubstituted heteroaryl. In certain embodiments, R⁴ is unsubstituted, 5- or 6-membered, monocyclic heteroaryl. In certain embodiments, R⁴ is substituted, 5- or 6-membered, monocyclic heteroaryl (e.g., substituted with halogen, unsubstituted alkyl, and/or —O-(unsubstituted alkyl)). In certain embodiments, R⁵ is hydrogen or substituted or unsubstituted alkyl. In certain embodiments, R⁵ is hydrogen. In certain embodiments, R⁵ is substituted or unsubstituted alkyl. In certain embodiments, R⁵ is substituted or unsubstituted C₁₋₄ alkyl. In certain embodiments, R⁵ is unsubstituted C₁₋₄ alkyl or C₁₋₄ haloalkyl. In certain embodiments, R⁵ is methyl or trifluoromethyl. In certain embodiments, R⁵ is methyl. In certain embodiments, R⁵ is trifluoromethyl. In certain embodiments, R⁵ is substituted or unsubstituted carbocyclyl (e.g., substituted or unsubstituted, monocyclic carbocyclyl). In certain embodiments, R⁵ is unsubstituted, cyclopropyl, cyclobutyl, cyclopentyl, cyclohexyl, or cycloheptyl. In certain embodiments, R⁵ is substituted, cyclopropyl, cyclobutyl, cyclopentyl, cyclohexyl, or cycloheptyl (e.g., substituted with halogen, unsubstituted alkyl, and/or —O-(unsubstituted alkyl)). In certain embodiments, R⁵ is substituted or unsubstituted aryl. In certain embodiments, R⁵ is unsubstituted phenyl. In certain embodiments, R⁵ is substituted phenyl (e.g., substituted with halogen, unsubstituted alkyl, and/or —O-(unsubstituted alkyl)). In certain embodiments, R⁵ is substituted or unsubstituted heteroaryl. In certain embodiments, R⁵ is unsubstituted, 5- or 6-membered, monocyclic heteroaryl. In certain embodiments, R⁵ is substituted, 5- or 6-membered, monocyclic heteroaryl (e.g., substituted with halogen, unsubstituted alkyl, and/or —O-(unsubstituted alkyl)).

In certain embodiments, A is

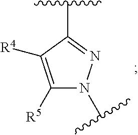

wherein R⁴ is hydrogen or substituted or unsubstituted alkyl; and R⁵ is hydrogen or substituted or unsubstituted alkyl. In certain embodiments, R⁴ is hydrogen or substituted or unsubstituted alkyl; and R⁵ is hydrogen. In certain embodiments, R⁴ is hydrogen; and R⁵ is hydrogen. In certain embodiments, R⁴ is substituted or unsubstituted alkyl; and R⁵ is hydrogen. In certain embodiments, R⁴ is substituted or unsubstituted C₁₋₄ alkyl; and R⁵ is hydrogen. In certain embodiments, R⁴ is unsubstituted C₁₋₄ alkyl or C₁₋₄ haloalkyl; and R⁵ is hydrogen. In certain embodiments, R⁴ is methyl or trifluoromethyl; and R⁵ is hydrogen. In certain embodiments, R⁴ is methyl; and R⁵ is hydrogen. In certain embodiments, R⁴ is trifluoromethyl; and R⁵ is hydrogen. In certain embodiments, R⁵ is substituted or unsubstituted alkyl; and R⁴ is hydrogen. In certain embodiments, R⁵ is substituted or unsubstituted C₁₋₄ alkyl; and R⁴ is hydrogen. In certain embodiments, R⁵ is unsubstituted C₁₋₄ alkyl or C₁₋₄ haloalkyl; and R⁴ is hydrogen. In certain embodiments, R⁵ is methyl or trifluoromethyl; and R⁴ is hydrogen. In certain embodiments, R⁵ is methyl; and R⁴ is hydrogen. In certain embodiments, R⁵ is trifluoromethyl; and R⁴ is hydrogen.

In certain embodiments, A is

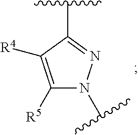

wherein R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl. In certain embodiments, R⁴ and R⁵ together with the atoms to which they are attached form a substituted or unsubstituted aryl (e.g., substituted or unsubstituted phenyl). In certain embodiments, R⁴ and R⁵ together with the atoms to which they are attached form an unsubstituted aryl. In certain embodiments, R⁴ and R⁵ together with the atoms to which they are attached form a substituted or unsubstituted heteroaryl (e.g., 5-6-membered, monocyclic, substituted or unsubstituted heteroaryl). In certain embodiments, A is

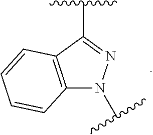 [SMILES: *c1nn(*)c2ccccc12]

In certain embodiments, A is

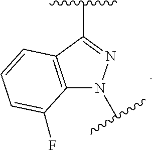 [SMILES: *c1nn(*)c2c(F)cccc12]

In certain embodiments, A is

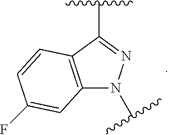 [SMILES: *c1nn(*)c2cc(F)ccc12]

In certain embodiments, A is

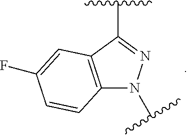 [SMILES: *c1nn(*)c2ccc(F)cc12]

In certain embodiments, A is

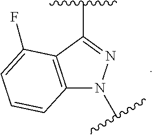 [SMILES: *c1nn(*)c2cccc(F)c12]

In certain embodiments, A is

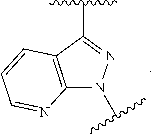 [SMILES: *c1nn(*)c2ncccc12]

In certain embodiments, A is

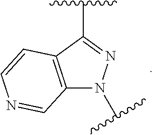 [SMILES: *c1nn(*)c2cnccc12]

In certain embodiments, A is

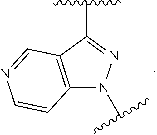 [SMILES: *c1nn(*)c2ccncc12]

In certain embodiments, A is

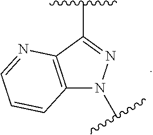 [SMILES: *c1nn(*)c2cccnc12]

In certain embodiments, A is

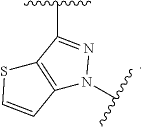 [SMILES: *c1nn(*)c2ccsc12]

In certain embodiments, A is

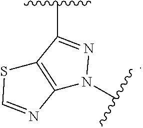 [SMILES: *c1nn(*)c2ncsc12]

In certain embodiments, A is

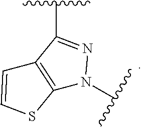 [SMILES: *c1nn(*)c2sccc12]

In certain embodiments, A is

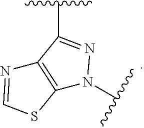 [SMILES: *c1nn(*)c2scnc12]

In certain embodiments, R⁴ is hydrogen, halogen, or substituted or unsubstituted alkyl. In certain embodiments, R⁴ is hydrogen. In certain embodiments, R⁴ is substituted or unsubstituted alkyl.

In certain embodiments, R⁵ is hydrogen, halogen, or substituted or unsubstituted alkyl. In certain embodiments, R⁵ is hydrogen. In certain embodiments, R⁵ is substituted or unsubstituted alkyl.

In certain embodiments, A is

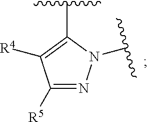

wherein R⁴ is hydrogen, halogen, substituted or unsubstituted alkyl, substituted or unsubstituted carbocyclyl, substituted or unsubstituted aryl, or substituted or unsubstituted heteroaryl; and R⁵ is hydrogen, halogen, substituted or unsubstituted alkyl, substituted or unsubstituted carbocyclyl, substituted or unsubstituted aryl, or substituted or unsubstituted heteroaryl. In certain embodiments, R⁴ is hydrogen or substituted or unsubstituted alkyl. In certain embodiments, R⁴ is hydrogen. In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ haloalkyl. In certain embodiments, R⁴ is methyl or trifluoromethyl. In certain embodiments, R⁴ is methyl. In certain embodiments, R⁴ is trifluoromethyl. In certain embodiments, R⁴ is substituted or unsubstituted carbocyclyl (e.g., substituted or unsubstituted, monocyclic carbocyclyl). In certain embodiments, R⁴ is unsubstituted, cyclopropyl, cyclobutyl, cyclopentyl, cyclohexyl, or cycloheptyl. In certain embodiments, R⁴ is substituted cyclopropyl, cyclobutyl, cyclopentyl, cyclohexyl, or cycloheptyl (e.g., substituted with halogen, unsubstituted alkyl, and/or —O-(unsubstituted alkyl)). In certain embodiments, R⁴ is substituted or unsubstituted aryl. In certain embodiments, R⁴ is unsubstituted phenyl. In certain embodiments, R⁴ is substituted phenyl (e.g., substituted with halogen, unsubstituted alkyl, and/or —O-(unsubstituted alkyl)). In certain embodiments, R⁴ is substituted or unsubstituted heteroaryl. In certain embodiments, R⁴ is unsubstituted, 5- or 6-membered, monocyclic heteroaryl. In certain embodiments, R⁴ is substituted, 5- or 6-membered, monocyclic heteroaryl (e.g., substituted with halogen, unsubstituted alkyl, and/or —O-(unsubstituted alkyl)). In certain embodiments, R⁵ is hydrogen or substituted or unsubstituted alkyl. In certain embodiments, R⁵ is hydrogen. In certain embodiments, R⁵ is substituted or unsubstituted alkyl. In certain embodiments, R⁵ is substituted or unsubstituted C₁₋₄ alkyl. In certain embodiments, R⁵ is unsubstituted C₁₋₄ alkyl or C₁₋₄ haloalkyl. In certain embodiments, R⁵ is methyl or trifluoromethyl. In certain embodiments, R⁵ is methyl. In certain embodiments, R⁵ is trifluoromethyl. In certain embodiments, R⁵ is substituted or unsubstituted carbocyclyl (e.g., substituted or unsubstituted, monocyclic carbocyclyl). In certain embodiments, R⁵ is unsubstituted, cyclopropyl, cyclobutyl, cyclopentyl, cyclohexyl, or cycloheptyl. In certain embodiments, R⁵ is substituted, cyclopropyl, cyclobutyl, cyclopentyl, cyclohexyl, or cycloheptyl (e.g., substituted with halogen, unsubstituted alkyl, and/or —O-(unsubstituted alkyl)). In certain embodiments, R⁵ is substituted or unsubstituted aryl. In certain embodiments, R⁵ is unsubstituted phenyl. In certain embodiments, R⁵ is substituted phenyl (e.g., substituted with halogen, unsubstituted alkyl, and/or —O-(unsubstituted alkyl)). In certain embodiments, R⁵ is substituted or unsubstituted heteroaryl. In certain embodiments, R⁵ is unsubstituted, 5- or 6-membered, monocyclic heteroaryl. In certain embodiments, R⁵ is substituted, 5- or 6-membered, monocyclic heteroaryl (e.g., substituted with halogen, unsubstituted alkyl, and/or —O-(unsubstituted alkyl)). In certain embodiments, R⁵ is trifluoromethyl; and R⁴ is hydrogen.

In certain embodiments, A is

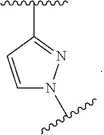 [SMILES: *c1ccn(*)n1]

In certain embodiments, A is

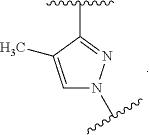 [SMILES: *c1nn(*)cc1C]

In certain embodiments, A is

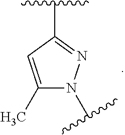 [SMILES: *c1cc(C)n(*)n1]

In certain embodiments, A is

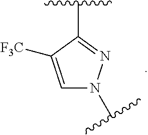 [SMILES: *c1nn(*)cc1C(F)(F)F]

In certain embodiments, A is

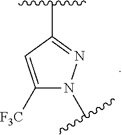 [SMILES: *c1cc(C(F)(F)F)n(*)n1]

In certain embodiments, A is

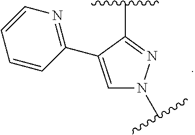 [SMILES: *c1nn(*)cc1-c1ccccn1]

In certain embodiments, A is

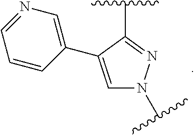 [SMILES: *c1nn(*)cc1-c1cccnc1]

In certain embodiments, A is

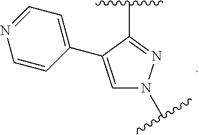 [SMILES: *c1nn(*)cc1-c1ccncc1]

In certain embodiments, A is

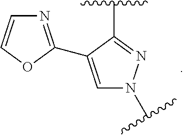 [SMILES: *c1nn(*)cc1-c1ncco1]

In certain embodiments, A is

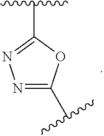 [SMILES: *c1nnc(*)o1]

In certain embodiments, A is

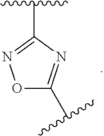 [SMILES: *c1noc(*)n1]

In certain embodiments, A is

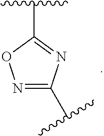 [SMILES: *c1noc(*)n1]

In certain embodiments, A is

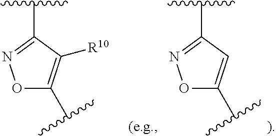

In certain embodiments, R¹⁰ is hydrogen, halogen, or substituted or unsubstituted alkyl. In certain embodiments, R¹⁰ is hydrogen or substituted or unsubstituted alkyl. In certain embodiments, R¹⁰ is substituted or unsubstituted alkyl. In certain embodiments, R¹⁰ is substituted or unsubstituted C₁₋₄ alkyl. In certain embodiments, R¹⁰ is hydrogen. In certain embodiments, R¹⁰ is substituted or unsubstituted carbocyclyl (e.g., substituted or unsubstituted, monocyclic carbocyclyl). In certain embodiments, R¹⁰ is unsubstituted, cyclopropyl, cyclobutyl, cyclopentyl, cyclohexyl, or cycloheptyl. In certain embodiments, R¹⁰ is substituted, cyclopropyl, cyclobutyl, cyclopentyl, cyclohexyl, or cycloheptyl (e.g., substituted with halogen, unsubstituted alkyl, and/or —O-(unsubstituted alkyl)). In certain embodiments, R¹⁰ is substituted or unsubstituted aryl. In certain embodiments, R¹⁰ is unsubstituted phenyl. In certain embodiments, R¹⁰ is substituted phenyl (e.g., substituted with halogen, unsubstituted alkyl, and/or —O-(unsubstituted alkyl)). In certain embodiments, R¹⁰ is substituted or unsubstituted heteroaryl. In certain embodiments, R¹⁰ is unsubstituted, 5- or 6-membered, monocyclic heteroaryl. In certain embodiments, R¹⁰ is substituted, 5- or 6-membered, monocyclic heteroaryl (e.g., substituted with halogen, unsubstituted alkyl, and/or —O-(unsubstituted alkyl)).

### Certain Embodiments

In certain embodiments, the compound of Formula (I) is of Formula (I-a):

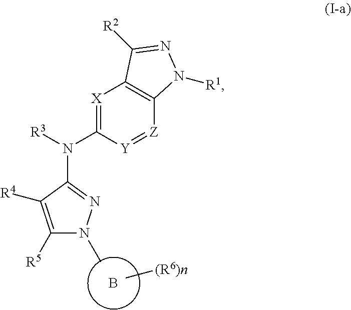

or a pharmaceutically acceptable salt, co-crystal, tautomer, stereoisomer, solvate, hydrate, polymorph, isotopically enriched compound, or prodrug thereof, wherein R¹, R², R³, R⁴, R⁵, R⁶, X, Y, Z, B, and n are as defined herein.

In certain embodiments, the compound of Formula (I) is of Formula (I-a-1):

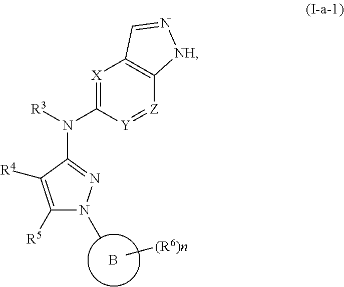

or a pharmaceutically acceptable salt, co-crystal, tautomer, stereoisomer, solvate, hydrate, polymorph, isotopically enriched compound, or prodrug thereof, wherein R³, R⁴, R⁵, R⁶, X, Y, Z, B, and n are as defined herein.

In certain embodiments, the compound of Formula (I) is of Formula (I-a-2):

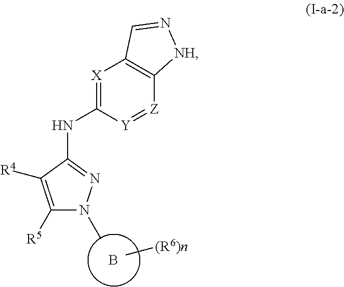

or a pharmaceutically acceptable salt, co-crystal, tautomer, stereoisomer, solvate, hydrate, polymorph, isotopically enriched compound, or prodrug thereof, wherein R⁴, R⁵, R⁶, X, Y, Z, B, and n are as defined herein.

In certain embodiments, the compound of Formula (I) is of Formula (I-a-3):

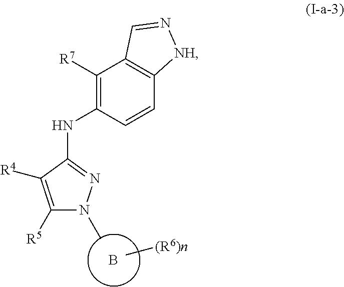

or a pharmaceutically acceptable salt, co-crystal, tautomer, stereoisomer, solvate, hydrate, polymorph, isotopically enriched compound, or prodrug thereof; wherein R⁴, R⁵, R⁶, R⁷, B, and n are as defined herein.

In certain embodiments, the compound of Formula (I) is of Formula (I-a-4):

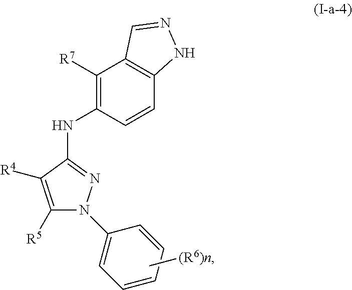

or a pharmaceutically acceptable salt, co-crystal, tautomer, stereoisomer, solvate, hydrate, polymorph, isotopically enriched compound, or prodrug thereof, wherein R⁴, R⁵, R⁶, R⁷, and n are as defined herein.

In certain embodiments, the compound of Formula (I) is of Formula (I-a-4a):

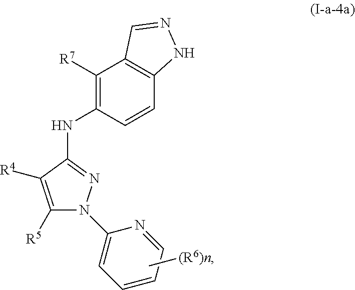

or a pharmaceutically acceptable salt, co-crystal, tautomer, stereoisomer, solvate, hydrate, polymorph, isotopically enriched compound, or prodrug thereof, wherein R⁴, R⁵, R⁶, R⁷, and n are as defined herein.

In certain embodiments, the compound of Formula (I) is of Formula (I-a-5):

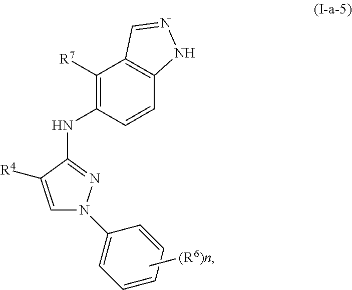

or a pharmaceutically acceptable salt, co-crystal, tautomer, stereoisomer, solvate, hydrate, polymorph, isotopically enriched compound, or prodrug thereof, wherein R⁴, R⁶, R⁷, and n are as defined herein.

In certain embodiments, the compound of Formula (I) is of Formula (I-a-5a):

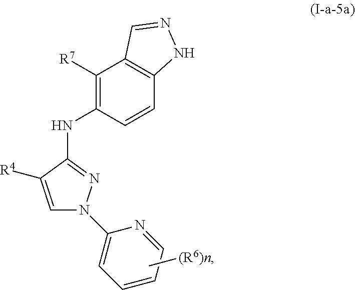

or a pharmaceutically acceptable salt, co-crystal, tautomer, stereoisomer, solvate, hydrate, polymorph, isotopically enriched compound, or prodrug thereof, wherein R⁴, R⁶, R⁷, and n are as defined herein.

In certain embodiments, the compound of Formula (I) is of Formula (I-a-6):

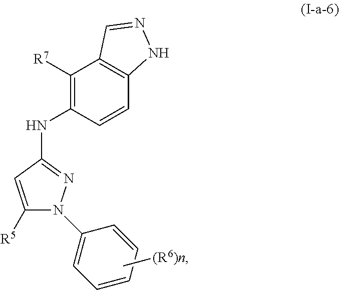

or a pharmaceutically acceptable salt, co-crystal, tautomer, stereoisomer, solvate, hydrate, polymorph, isotopically enriched compound, or prodrug thereof, wherein R⁴, R⁵, R⁷, and n are as defined herein.

In certain embodiments, the compound of Formula (I) is of Formula (I-a-6a):

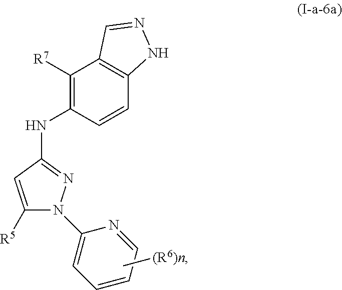

or a pharmaceutically acceptable salt, co-crystal, tautomer, stereoisomer, solvate, hydrate, polymorph, isotopically enriched compound, or prodrug thereof, wherein R⁴, R⁵, R⁷, and n are as defined herein.

In certain embodiments, the compound of Formula (I) is of Formula (I-a-7):

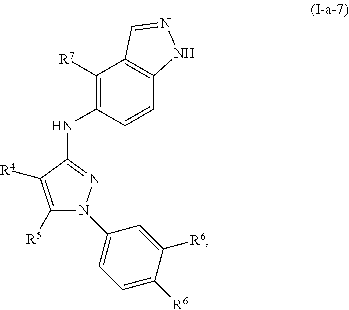

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-7a):

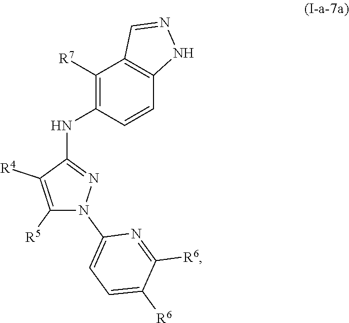

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-8):

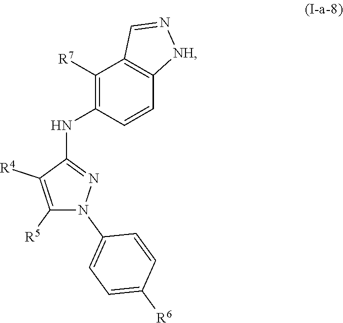

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-8a):

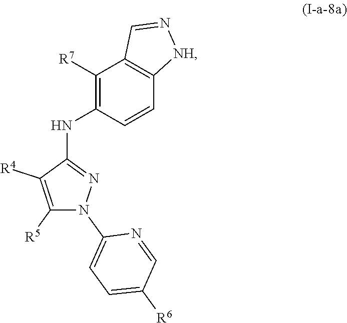

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-9):

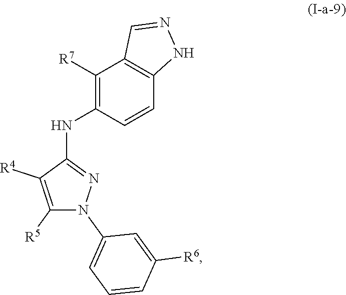

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-9a):

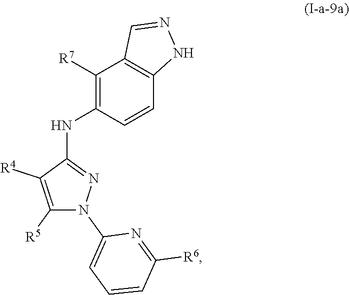

or a pharmaceutically acceptable salt, co-crystal, tautomer, stereoisomer, solvate, hydrate, polymorph, isotopically enriched compound, or prodrug thereof, wherein R⁴, R⁵, R⁶, and R⁷ are as defined herein.

In certain embodiments, the compound of Formula (I) is of Formula (I-a-10):

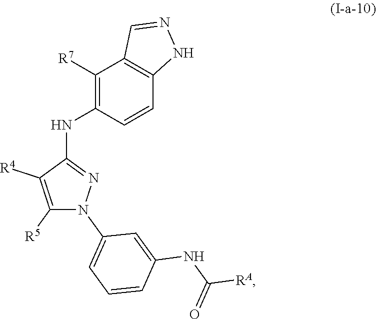

or a pharmaceutically acceptable salt, co-crystal, tautomer, stereoisomer, solvate, hydrate, polymorph, isotopically enriched compound, or prodrug thereof, wherein R⁴, R⁵, R⁷, and R^{A} are as defined herein.

In certain embodiments, the compound of Formula (I) is of Formula (I-a-10a):

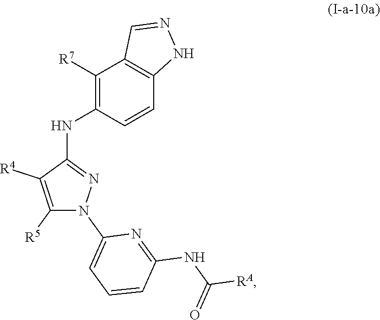

or a pharmaceutically acceptable salt, co-crystal, tautomer, stereoisomer, solvate, hydrate, polymorph, isotopically enriched compound, or prodrug thereof, wherein R⁴, R⁵, R⁷, and R^{A} are as defined herein.

In certain embodiments, the compound of Formula (I) is of Formula (I-b):

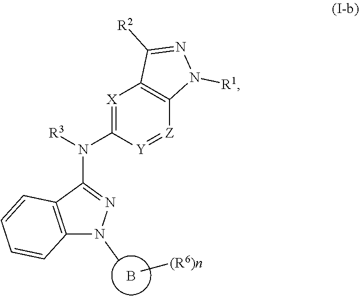

or a pharmaceutically acceptable salt, co-crystal, tautomer, stereoisomer, solvate, hydrate, polymorph, isotopically enriched compound, or prodrug thereof, wherein R¹, R², R³, R⁶, X, Y, Z, B, and n are as defined herein.

In certain embodiments, the compound of Formula (I) is of Formula (I-b-1):

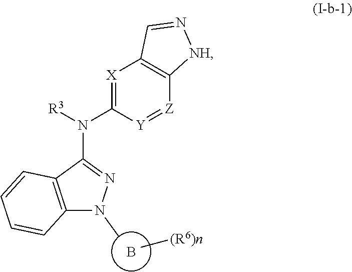

or a pharmaceutically acceptable salt, co-crystal, tautomer, stereoisomer, solvate, hydrate, polymorph, isotopically enriched compound, or prodrug thereof, wherein R³, R⁶, X, Y, Z, B, and n are as defined herein.

In certain embodiments, the compound of Formula (I) is of Formula (I-b-2):

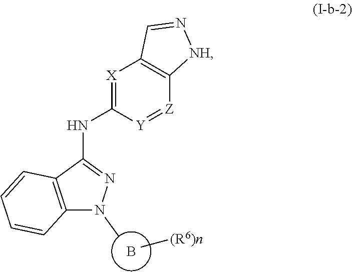

or a pharmaceutically acceptable salt, co-crystal, tautomer, stereoisomer, solvate, hydrate, polymorph, isotopically enriched compound, or prodrug thereof, wherein R⁶, X, Y, Z, B, and n are as defined herein.

In certain embodiments, the compound of Formula (I) is of Formula (I-b-3):

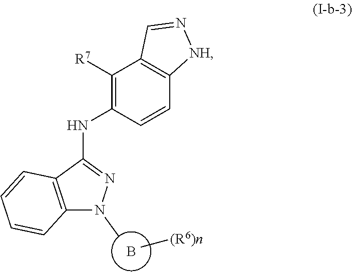

or a pharmaceutically acceptable salt, co-crystal, tautomer, stereoisomer, solvate, hydrate, polymorph, isotopically enriched compound, or prodrug thereof, wherein R⁷, R⁶, B, and n are as defined herein.

In certain embodiments, the compound of Formula (I) is of Formula (I-b-4):

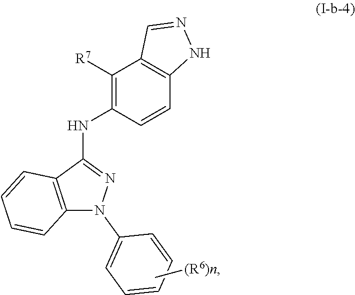

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-b-4a):

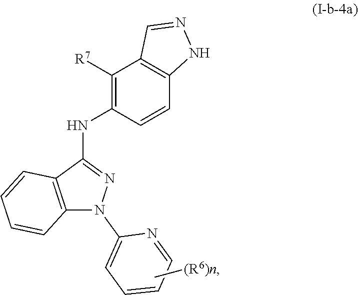

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-b-5):

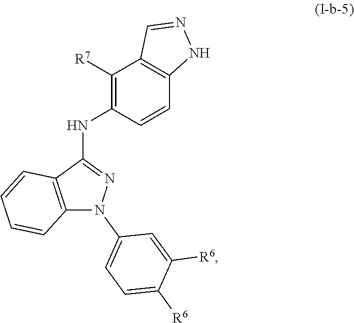

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-5a):

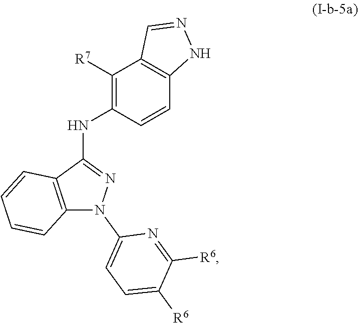

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-6):

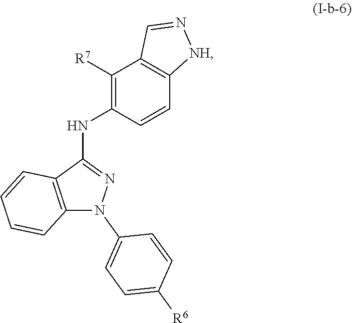

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-6a):

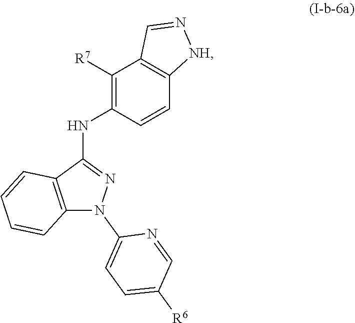

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-7):

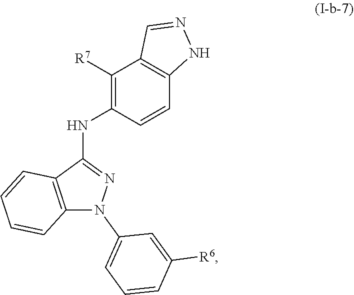

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-7a):

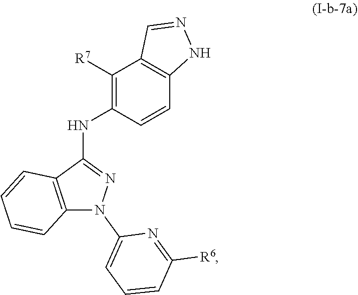

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-b-8):

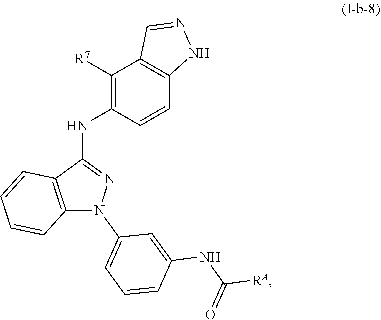

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-b-8a):

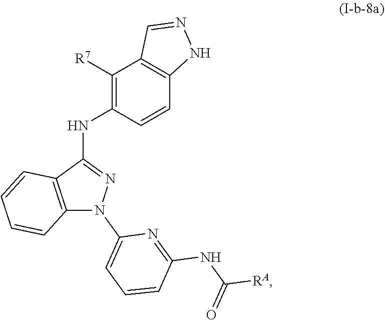

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-c):

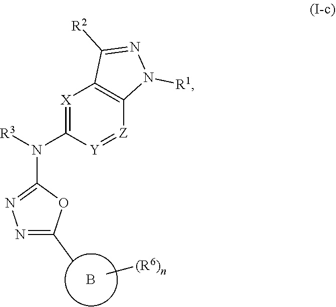

or a pharmaceutically acceptable salt, co-crystal, tautomer, stereoisomer, solvate, hydrate, polymorph, isotopically enriched compound, or prodrug thereof, wherein R¹, R², R³, R⁶, X, Y, Z, B, and n are as defined herein.

In certain embodiments, the compound of Formula (I) is of Formula (I-c-1):

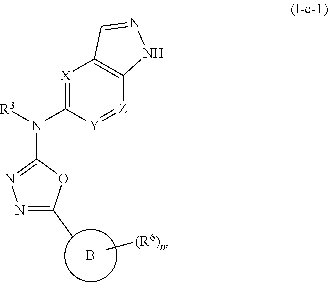

or a pharmaceutically acceptable salt, co-crystal, tautomer, stereoisomer, solvate, hydrate, polymorph, isotopically enriched compound, or prodrug thereof, wherein R³, R⁶, X, Y, Z, B, and n are as defined herein.

In certain embodiments, the compound of Formula (I) is of Formula (I-c-2):

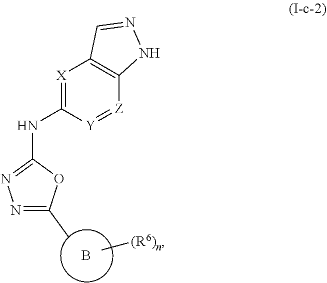

or a pharmaceutically acceptable salt, co-crystal, tautomer, stereoisomer, solvate, hydrate, polymorph, isotopically enriched compound, or prodrug thereof, wherein R⁶, X, Y, Z, B, and n are as defined herein.

In certain embodiments, the compound of Formula (I) is of Formula (I-c-3):

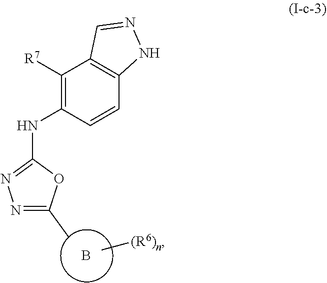

or a pharmaceutically acceptable salt, co-crystal, tautomer, stereoisomer, solvate, hydrate, polymorph, isotopically enriched compound, or prodrug thereof, wherein R⁷, R⁶, B, and n are as defined herein.

In certain embodiments, the compound of Formula (I) is of Formula (I-c-4):

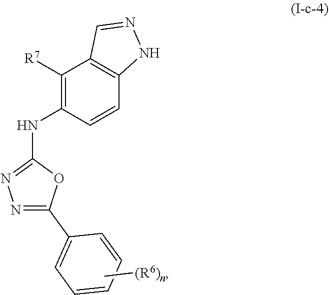

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-c-4a):

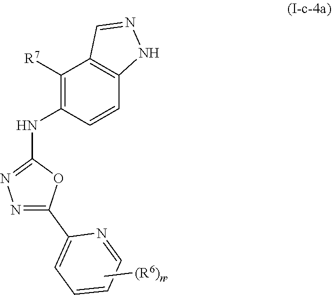

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-c-5):

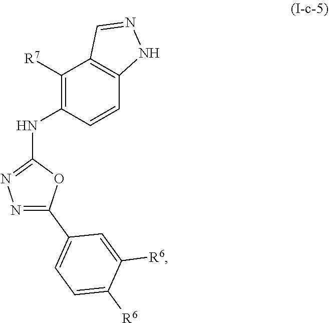

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-5a):

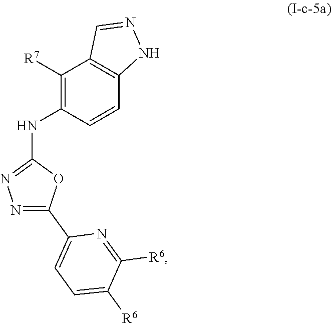

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-6):

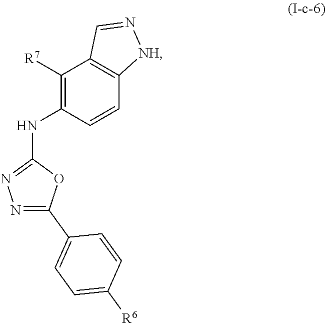

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-6a):

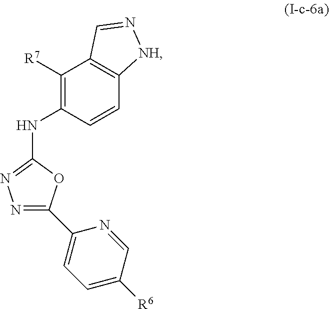

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-7):

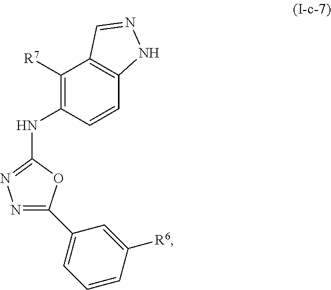

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-7a):

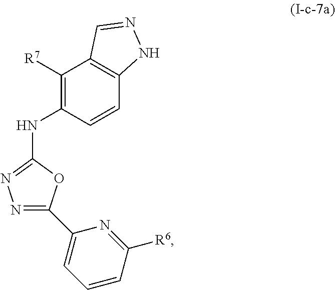

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-c-8):

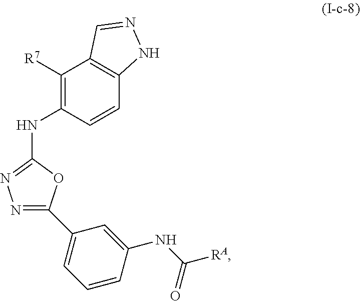

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-c-8a):

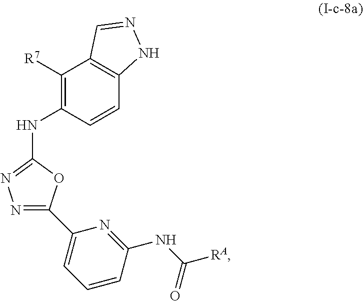

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-d):

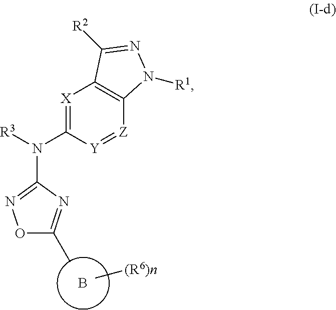

or a pharmaceutically acceptable salt, co-crystal, tautomer, stereoisomer, solvate, hydrate, polymorph, isotopically enriched compound, or prodrug thereof, wherein R¹, R², R³, R⁶, X, Y, Z, B, and n are as defined herein.

In certain embodiments, the compound of Formula (I) is of Formula (I-d-1):

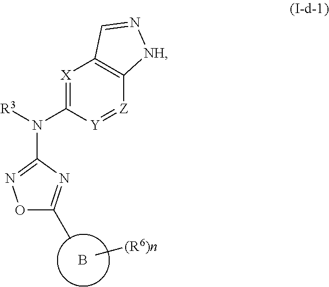

or a pharmaceutically acceptable salt, co-crystal, tautomer, stereoisomer, solvate, hydrate, polymorph, isotopically enriched compound, or prodrug thereof, wherein R³, R⁶, X, Y, Z, B, and n are as defined herein.

In certain embodiments, the compound of Formula (I) is of Formula (I-d-2):

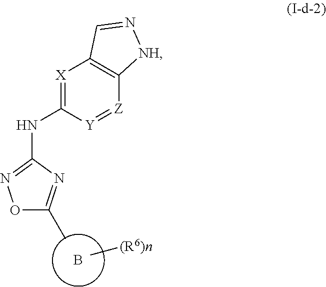

or a pharmaceutically acceptable salt, co-crystal, tautomer, stereoisomer, solvate, hydrate, polymorph, isotopically enriched compound, or prodrug thereof, wherein R⁶, X, Y, Z, B, and n are as defined herein.

In certain embodiments, the compound of Formula (I) is of Formula (I-d-3):

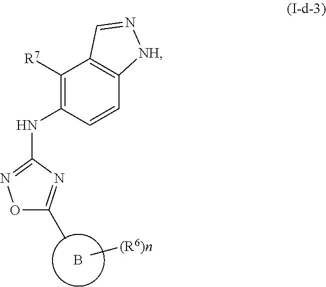

or a pharmaceutically acceptable salt, co-crystal, tautomer, stereoisomer, solvate, hydrate, polymorph, isotopically enriched compound, or prodrug thereof, wherein R⁷, R⁶, B, and n are as defined herein.

In certain embodiments, the compound of Formula (I) is of Formula (I-d-4):

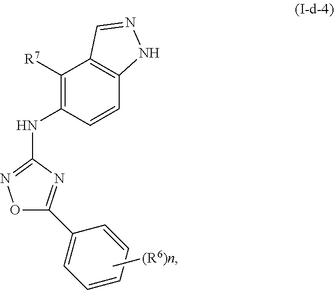

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-d-4a):

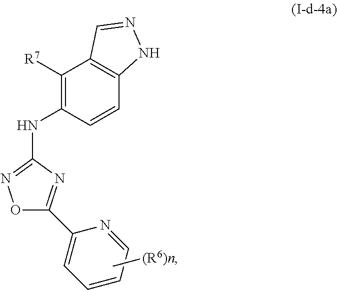

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-d-5):

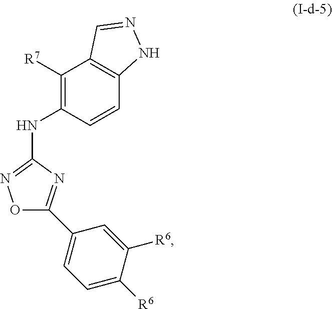

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-5a):

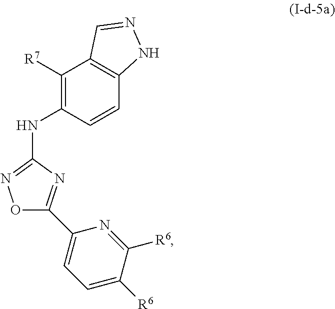

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-6):

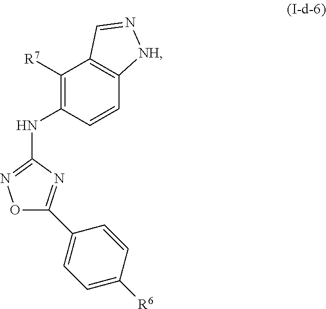

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-6a):

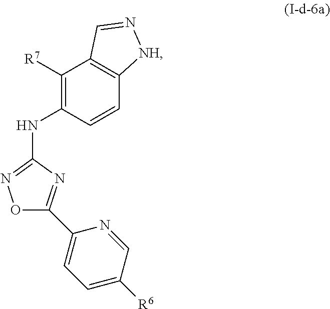

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-7):

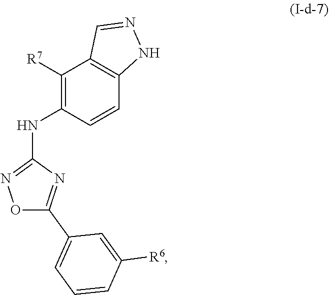

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-7a):

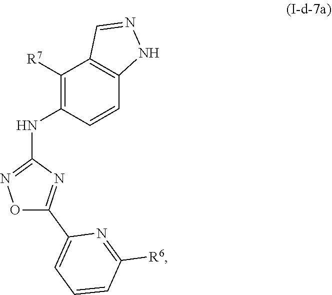

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-d-8):

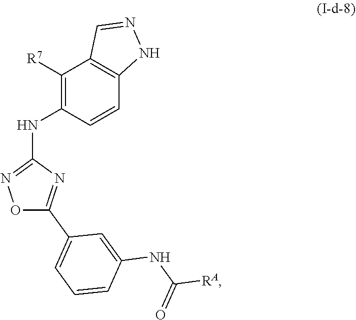

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-d-8a):

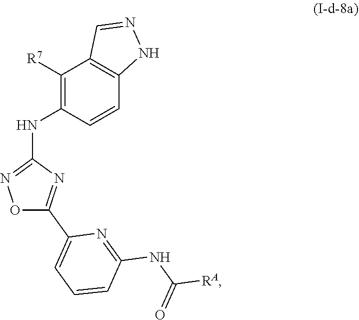

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-e):

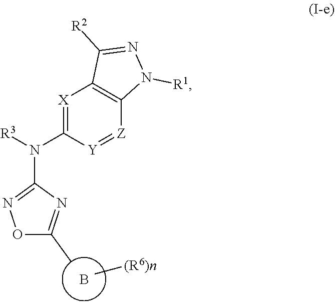

or a pharmaceutically acceptable salt, co-crystal, tautomer, stereoisomer, solvate, hydrate, polymorph, isotopically enriched compound, or prodrug thereof, wherein R¹, R², R³, R⁶, X, Y, Z, B, and n are as defined herein.

In certain embodiments, the compound of Formula (I) is of Formula (I-e-1):

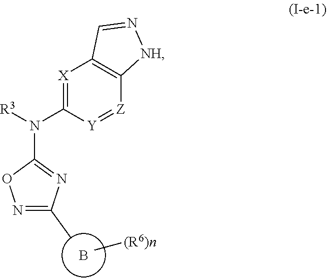

or a pharmaceutically acceptable salt, co-crystal, tautomer, stereoisomer, solvate, hydrate, polymorph, isotopically enriched compound, or prodrug thereof, wherein R³, R⁶, X, Y, Z, B, and n are as defined herein.

In certain embodiments, the compound of Formula (I) is of Formula (I-e-2):

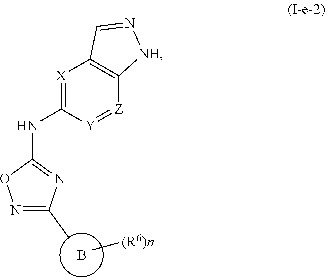

or a pharmaceutically acceptable salt, co-crystal, tautomer, stereoisomer, solvate, hydrate, polymorph, isotopically enriched compound, or prodrug thereof, wherein R⁶, X, Y, Z, B, and n are as defined herein.

In certain embodiments, the compound of Formula (I) is of Formula (I-e-3):

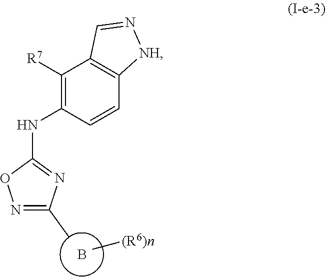

or a pharmaceutically acceptable salt, co-crystal, tautomer, stereoisomer, solvate, hydrate, polymorph, isotopically enriched compound, or prodrug thereof, wherein R⁷, R⁶, B, and n are as defined herein.

In certain embodiments, the compound of Formula (I) is of Formula (I-e-4):

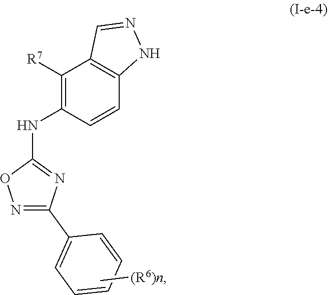

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-e-4a):

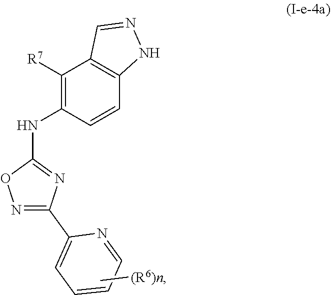

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-e-5):

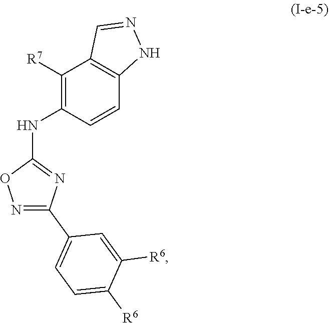

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-5a):

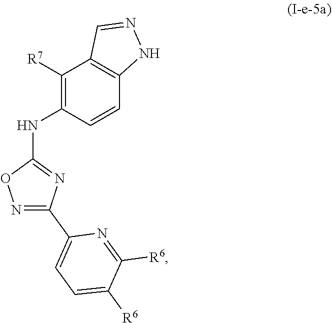

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-6):

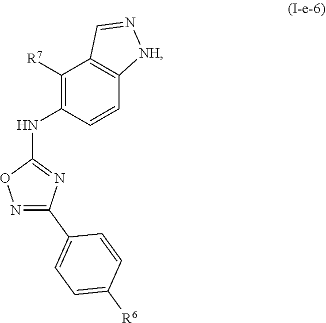

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-6a):

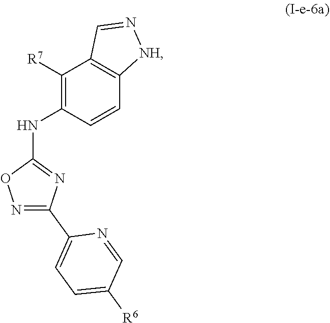

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-7):

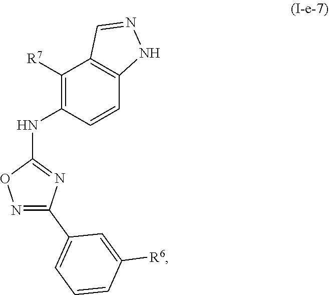

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-7a):

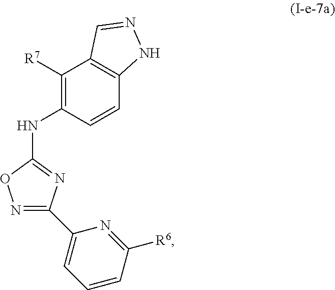

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-e-8):

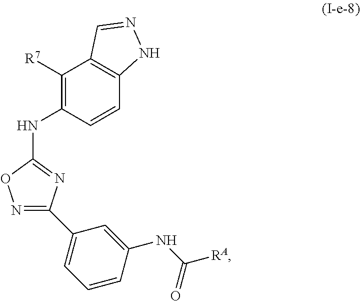

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-e-8a):

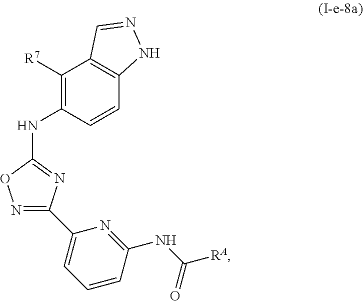

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-f):

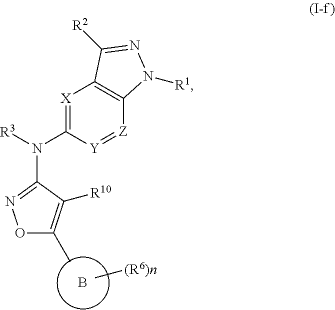

or a pharmaceutically acceptable salt, co-crystal, tautomer, stereoisomer, solvate, hydrate, polymorph, isotopically enriched compound, or prodrug thereof, wherein R¹, R², R³, R⁶, R¹⁰, X, Y, Z, B, and n are as defined herein.

In certain embodiments, the compound of Formula (I) is of Formula (I-f-1):

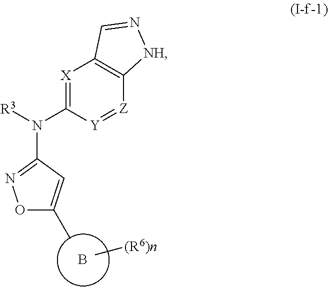

or a pharmaceutically acceptable salt, co-crystal, tautomer, stereoisomer, solvate, hydrate, polymorph, isotopically enriched compound, or prodrug thereof, wherein R³, R⁶, X, Y, Z, B, and n are as defined herein.

In certain embodiments, the compound of Formula (I) is of Formula (I-f-2):

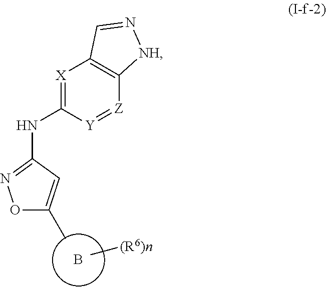

or a pharmaceutically acceptable salt, co-crystal, tautomer, stereoisomer, solvate, hydrate, polymorph, isotopically enriched compound, or prodrug thereof, wherein R⁶, X, Y, Z, B, and n are as defined herein.

In certain embodiments, the compound of Formula (I) is of Formula (I-f-3):

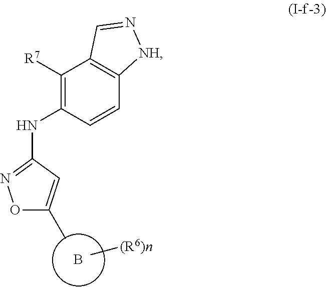

or a pharmaceutically acceptable salt, co-crystal, tautomer, stereoisomer, solvate, hydrate, polymorph, isotopically enriched compound, or prodrug thereof, wherein R⁷, R⁶, B, and n are as defined herein.

In certain embodiments, the compound of Formula (I) is of Formula (I-f-4):

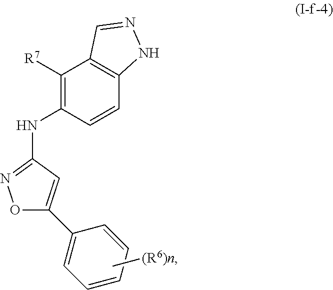

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-f-4a):

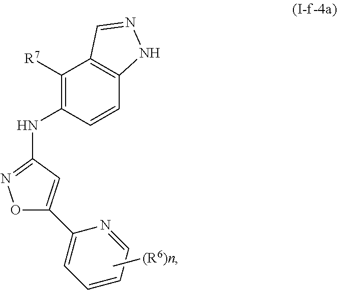

or a pharmaceutically acceptable salt, co-crystal, tautomer, stereoisomer, solvate, hydrate, polymorph, isotopically enriched compound, or prodrug thereof, wherein R⁷, R⁶, and n are as defined herein.

In certain embodiments, the compound of Formula (I) is of Formula (I-f-5):

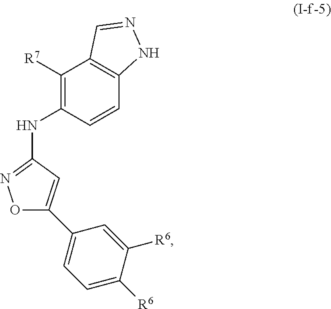

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-5a):

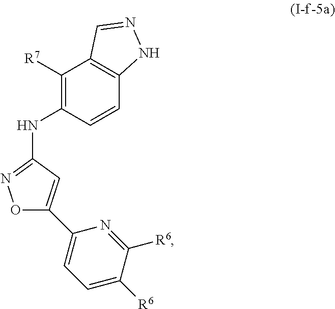

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-6):

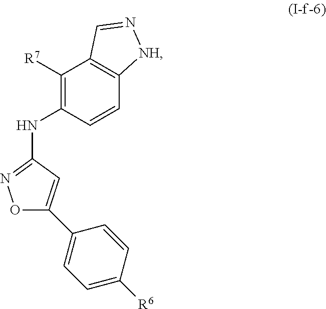

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-6a):

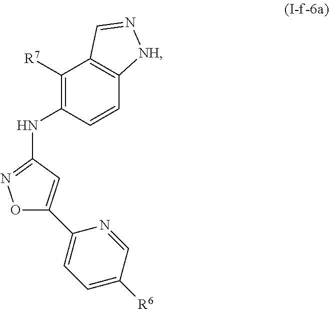

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-7):

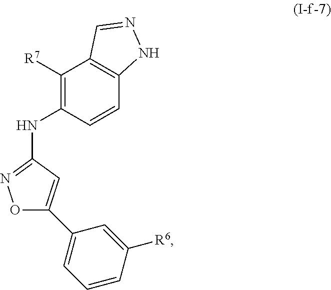

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-7a):

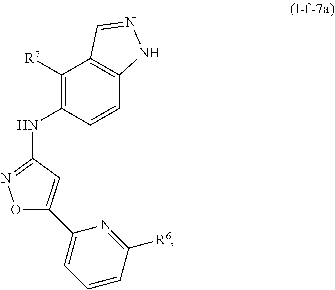

or a pharmaceutically acceptable salt, co-crystal, tautomer, stereoisomer, solvate, hydrate, polymorph, isotopically enriched compound, or prodrug thereof, wherein R⁶ and R⁷ are as defined herein.

In certain embodiments, the compound of Formula (I) is of Formula (I-f-8):

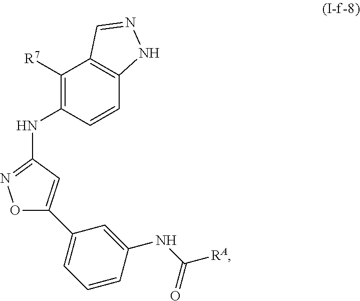

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is of Formula (I-f-8a):

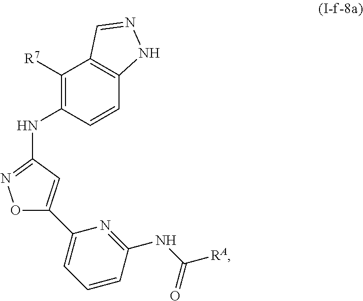

or a pharmaceutically acceptable salt, co-crystal, tautomer, stereoisomer, solvate, hydrate, polymorph, isotopically enriched compound, or prodrug thereof, wherein R^{A} and R⁷ are as defined herein.

In certain embodiments, the compound of Formula (I) is one of the following compounds, or a pharmaceutically acceptable salt, co-crystal, tautomer, stereoisomer, solvate, hydrate, polymorph, isotopically enriched compound, or prodrug thereof:

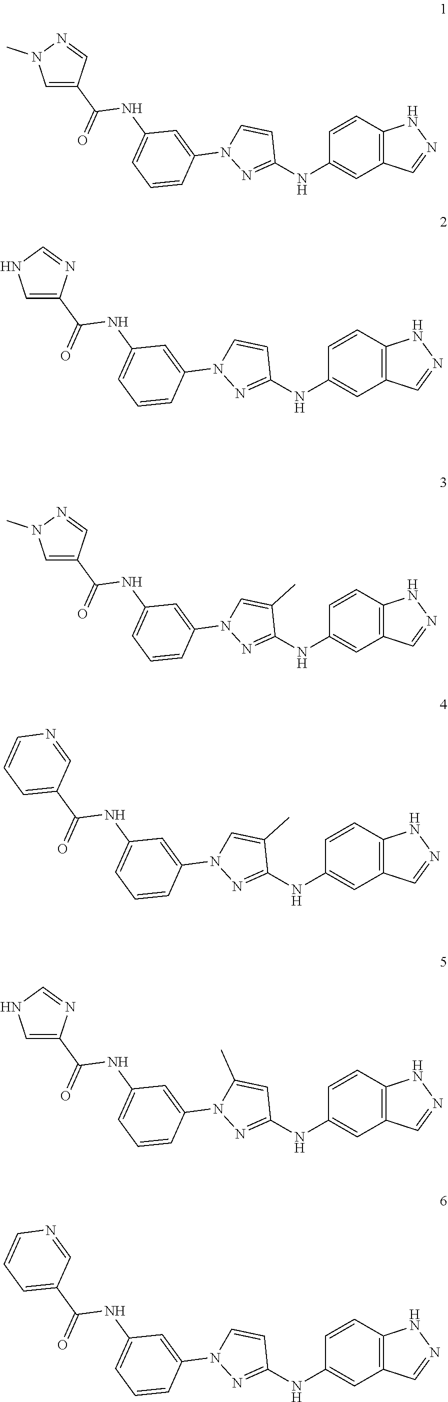

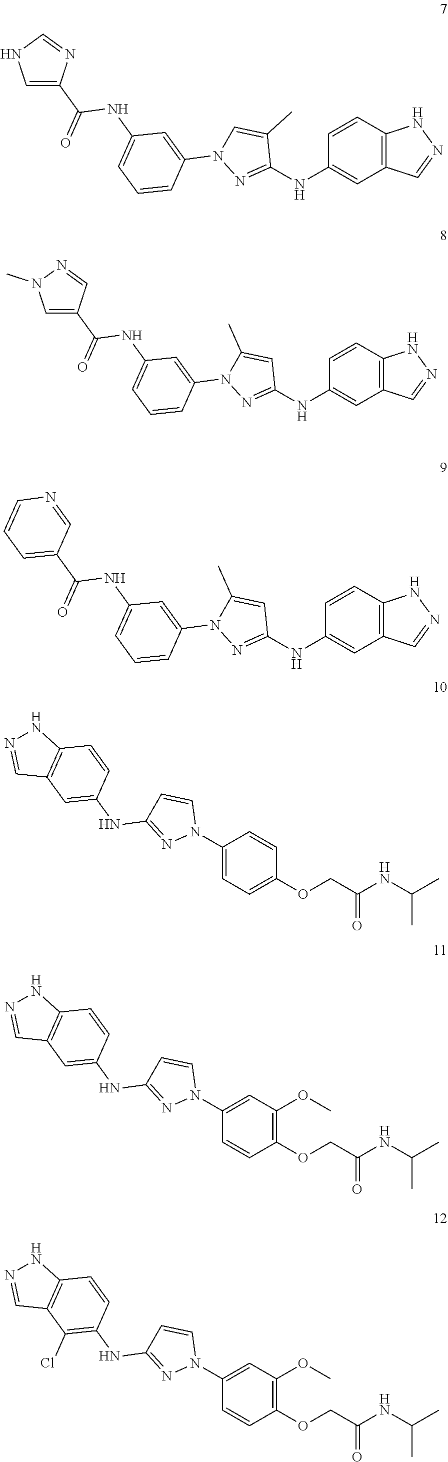

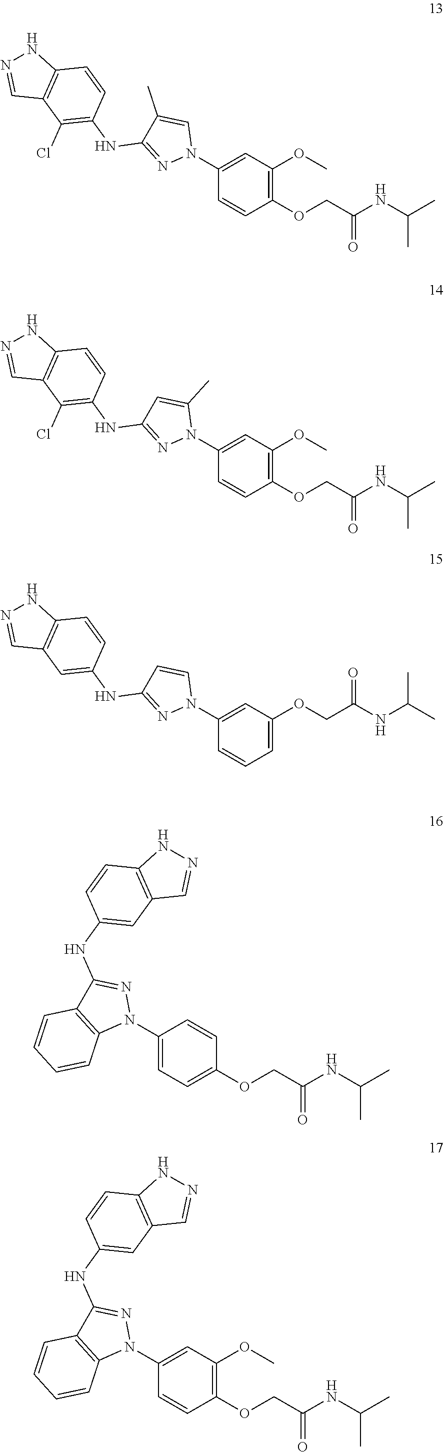 [SMILES: COc1cc(-n2cc(C)c(Nc3ccc4[nH]ncc4c3Cl)n2)ccc1OCC(=O)NC(C)C ; COc1cc(-n2nc(Nc3ccc4[nH]ncc4c3Cl)cc2C)ccc1OCC(=O)NC(C)C ; CC(C)NC(=O)COc1cccc(-n2ccc(Nc3ccc4[nH]ncc4c3)n2)c1 ; CC(C)NC(=O)COc1ccc(-n2nc(Nc3ccc4[nH]ncc4c3)c3ccccc32)cc1 ; COc1cc(-n2nc(Nc3ccc4[nH]ncc4c3)c3ccccc32)ccc1OCC(=O)NC(C)C]

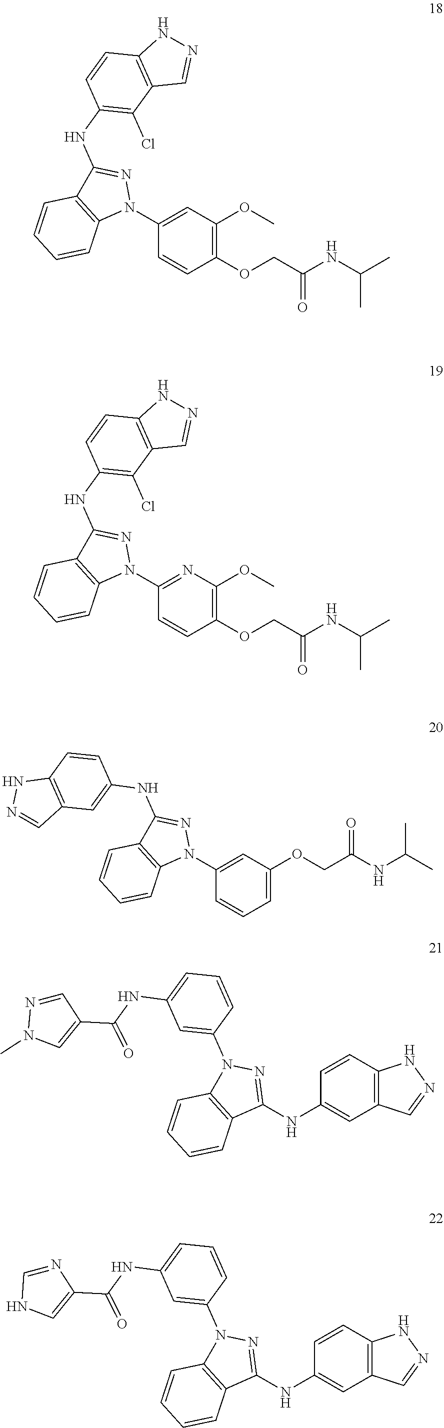

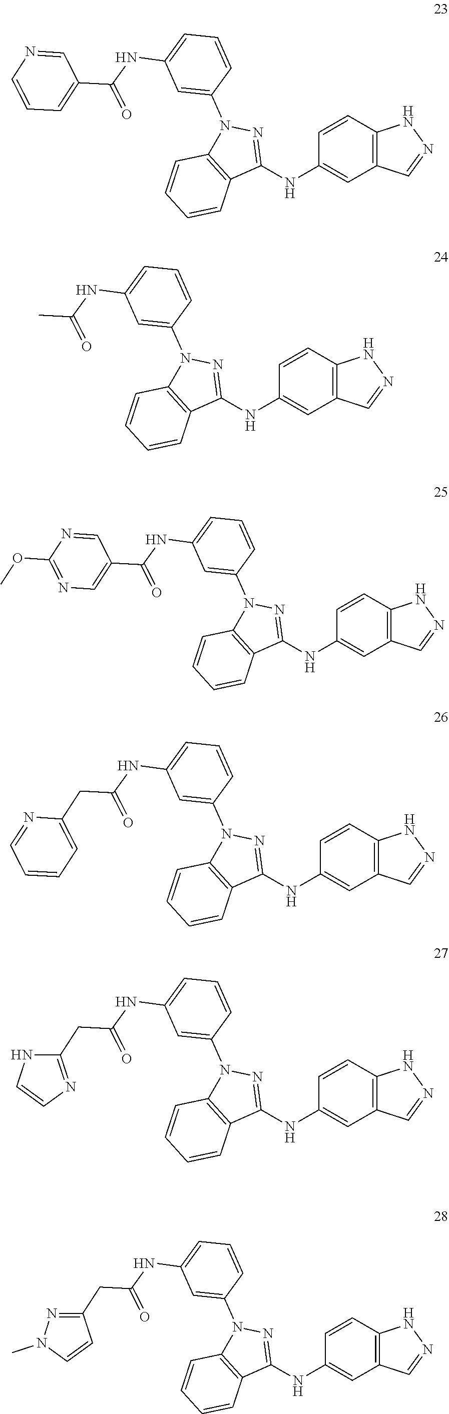 [SMILES: O=C(Nc1cccc(-n2nc(Nc3ccc4[nH]ncc4c3)c3ccccc32)c1)c1cccnc1 ; CC(=O)Nc1cccc(-n2nc(Nc3ccc4[nH]ncc4c3)c3ccccc32)c1 ; COc1ncc(C(=O)Nc2cccc(-n3nc(Nc4ccc5[nH]ncc5c4)c4ccccc43)c2)cn1 ; O=C(Cc1ccccn1)Nc1cccc(-n2nc(Nc3ccc4[nH]ncc4c3)c3ccccc32)c1 ; O=C(Cc1ncc[nH]1)Nc1cccc(-n2nc(Nc3ccc4[nH]ncc4c3)c3ccccc32)c1 ; Cn1ccc(CC(=O)Nc2cccc(-n3nc(Nc4ccc5[nH]ncc5c4)c4ccccc43)c2)n1]

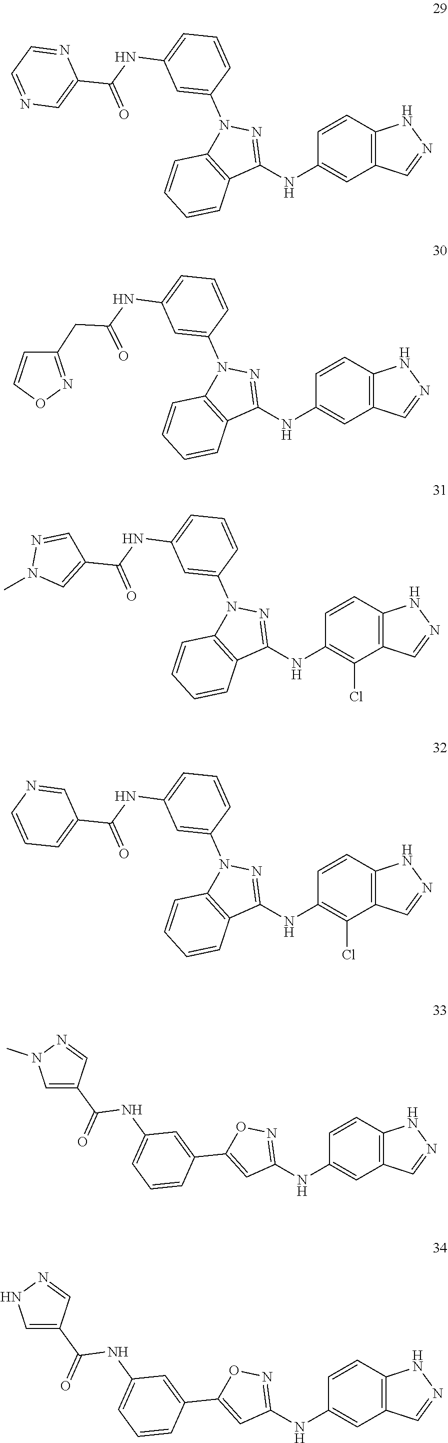 [SMILES: O=C(Nc1cccc(-n2nc(Nc3ccc4[nH]ncc4c3)c3ccccc32)c1)c1cnccn1 ; O=C(Cc1ccon1)Nc1cccc(-n2nc(Nc3ccc4[nH]ncc4c3)c3ccccc32)c1 ; Cn1cc(C(=O)Nc2cccc(-n3nc(Nc4ccc5[nH]ncc5c4Cl)c4ccccc43)c2)cn1 ; O=C(Nc1cccc(-n2nc(Nc3ccc4[nH]ncc4c3Cl)c3ccccc32)c1)c1cccnc1 ; Cn1cc(C(=O)Nc2cccc(-c3cc(Nc4ccc5[nH]ncc5c4)no3)c2)cn1 ; O=C(Nc1cccc(-c2cc(Nc3ccc4[nH]ncc4c3)no2)c1)c1cn[nH]c1]

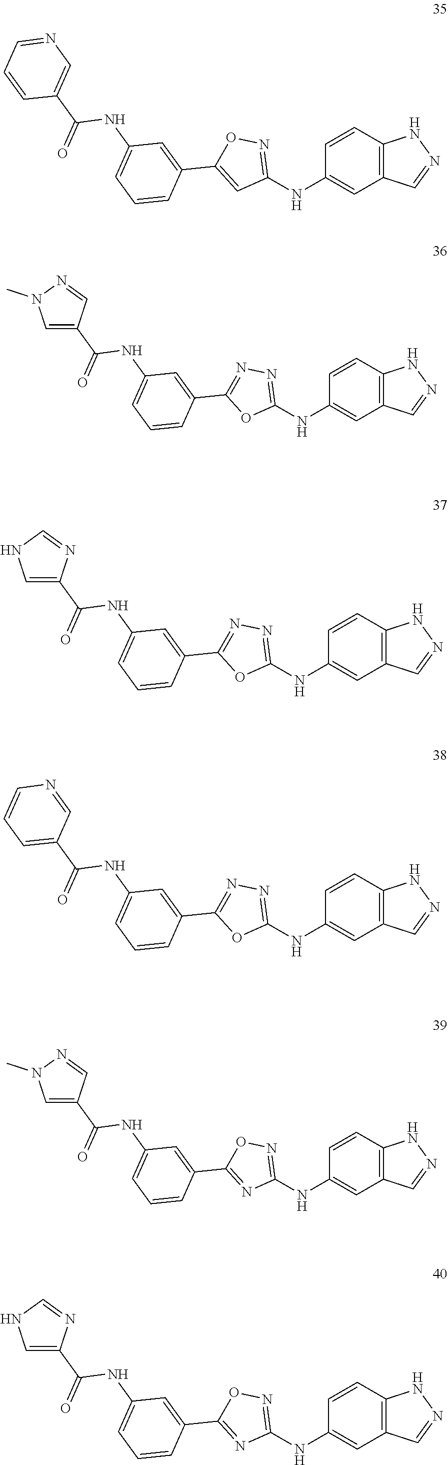 [SMILES: O=C(Nc1cccc(-c2cc(Nc3ccc4[nH]ncc4c3)no2)c1)c1cccnc1 ; Cn1cc(C(=O)Nc2cccc(-c3nnc(Nc4ccc5[nH]ncc5c4)o3)c2)cn1 ; O=C(Nc1cccc(-c2nnc(Nc3ccc4[nH]ncc4c3)o2)c1)c1c[nH]cn1 ; O=C(Nc1cccc(-c2nnc(Nc3ccc4[nH]ncc4c3)o2)c1)c1cccnc1 ; Cn1cc(C(=O)Nc2cccc(-c3nc(Nc4ccc5[nH]ncc5c4)no3)c2)cn1 ; O=C(Nc1cccc(-c2nc(Nc3ccc4[nH]ncc4c3)no2)c1)c1c[nH]cn1]

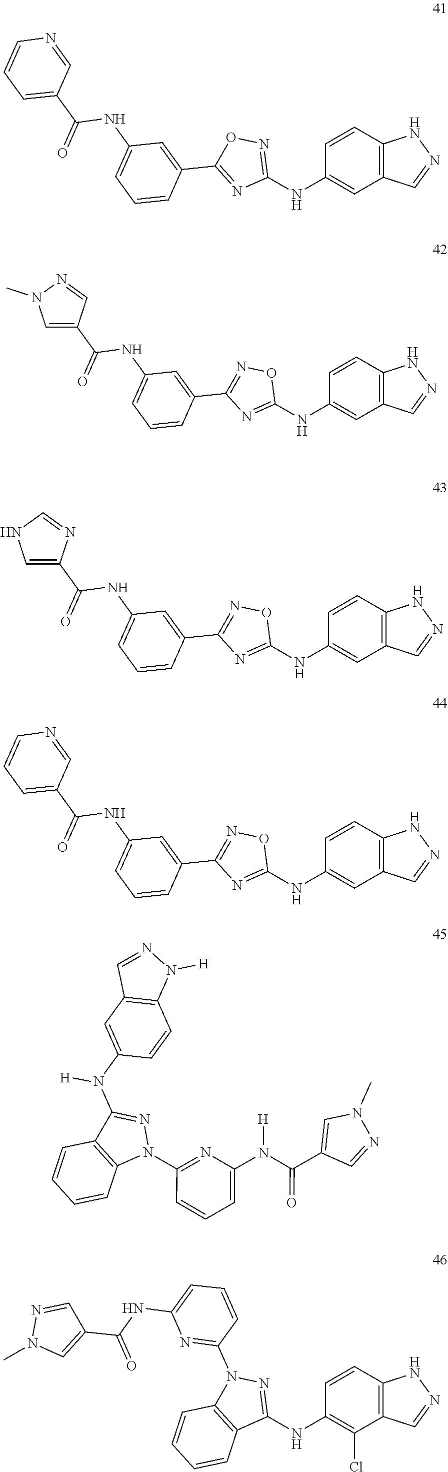 [SMILES: O=C(Nc1cccc(-c2nc(Nc3ccc4[nH]ncc4c3)no2)c1)c1cccnc1 ; Cn1cc(C(=O)Nc2cccc(-c3noc(Nc4ccc5[nH]ncc5c4)n3)c2)cn1 ; O=C(Nc1cccc(-c2noc(Nc3ccc4[nH]ncc4c3)n2)c1)c1c[nH]cn1 ; O=C(Nc1cccc(-c2noc(Nc3ccc4[nH]ncc4c3)n2)c1)c1cccnc1 ; Cn1cc(C(=O)Nc2cccc(-n3nc(Nc4ccc5[nH]ncc5c4)c4ccccc43)n2)cn1 ; Cn1cc(C(=O)Nc2cccc(-n3nc(Nc4ccc5[nH]ncc5c4Cl)c4ccccc43)n2)cn1]

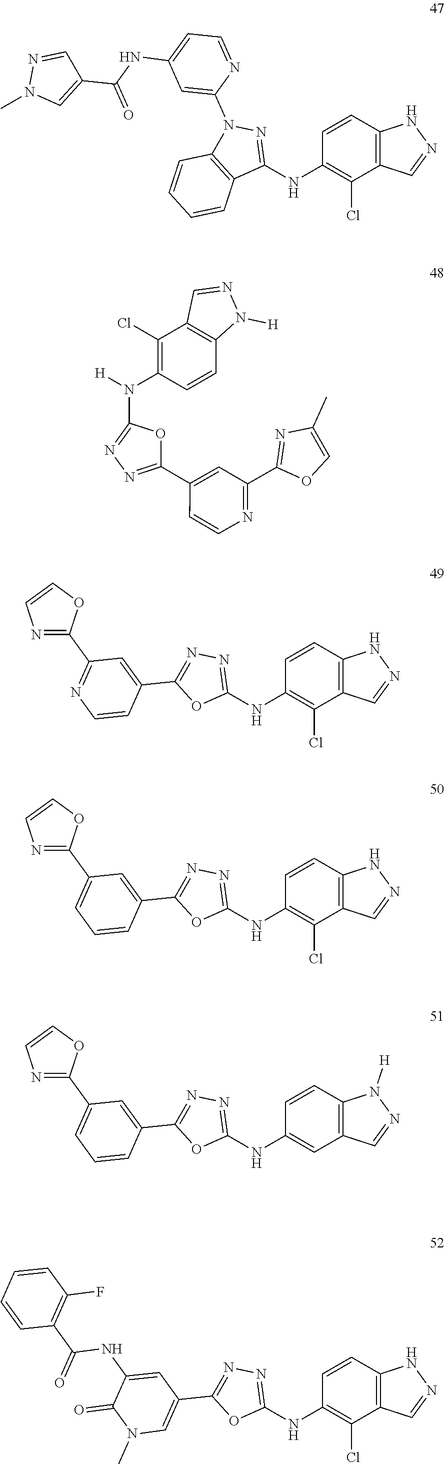 [SMILES: Cn1cc(C(=O)Nc2ccnc(-n3nc(Nc4ccc5[nH]ncc5c4Cl)c4ccccc43)c2)cn1 ; Cc1coc(-c2cc(-c3nnc(Nc4ccc5[nH]ncc5c4Cl)o3)ccn2)n1 ; Clc1c(Nc2nnc(-c3ccnc(-c4ncco4)c3)o2)ccc2[nH]ncc12 ; Clc1c(Nc2nnc(-c3cccc(-c4ncco4)c3)o2)ccc2[nH]ncc12 ; c1cc(-c2ncco2)cc(-c2nnc(Nc3ccc4[nH]ncc4c3)o2)c1 ; Cn1cc(-c2nnc(Nc3ccc4[nH]ncc4c3Cl)o2)cc(NC(=O)c2ccccc2F)c1=O]

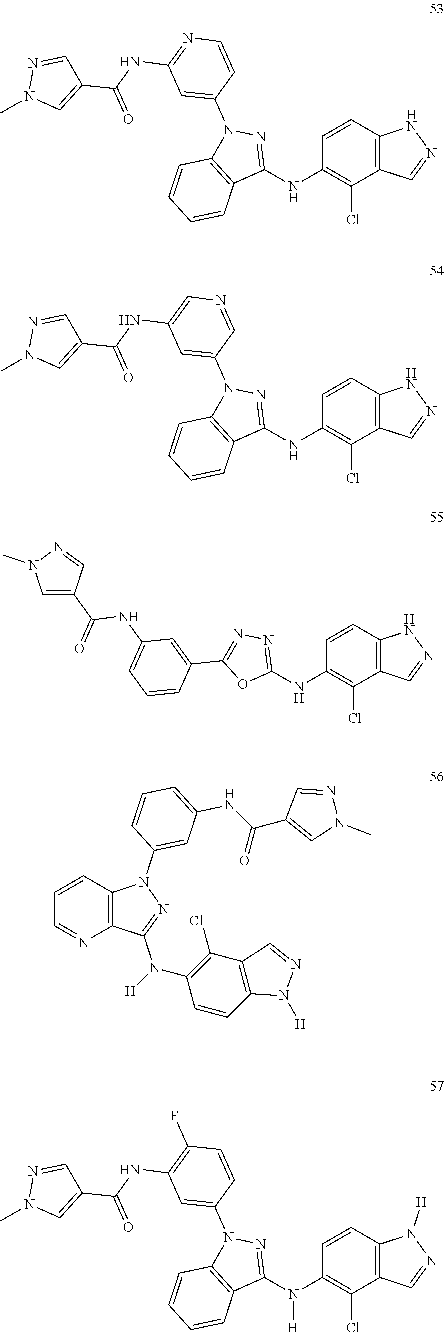

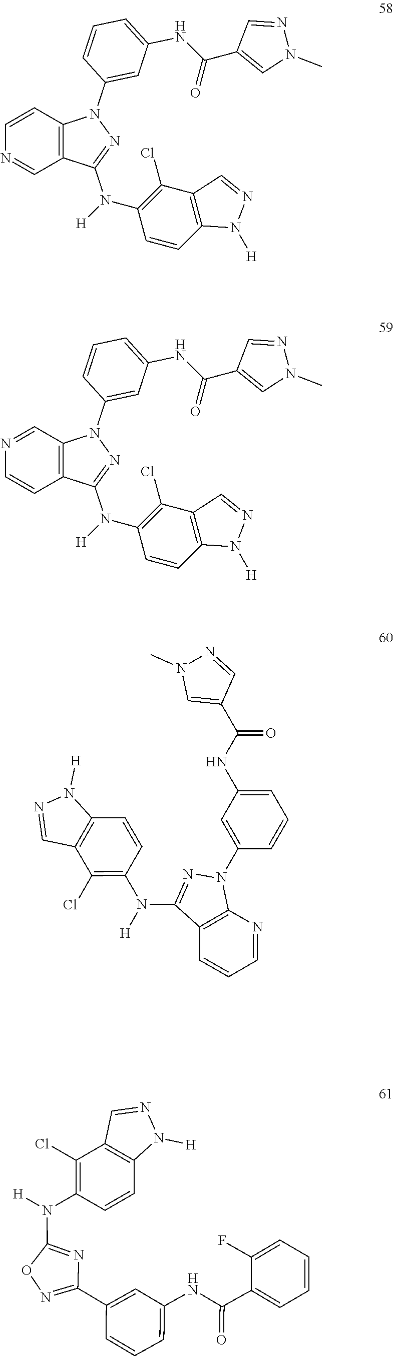

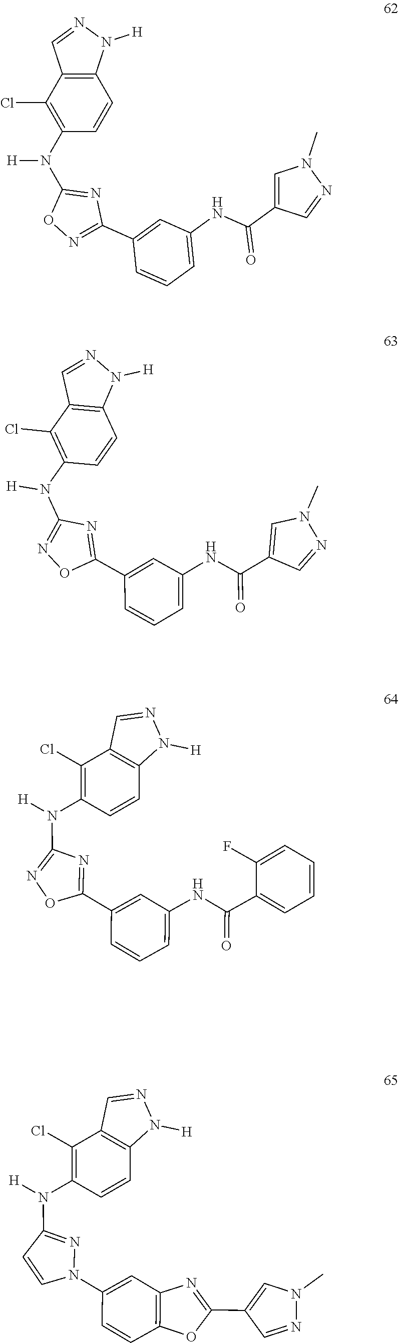 [SMILES: Cn1cc(C(=O)Nc2cccc(-c3noc(Nc4ccc5[nH]ncc5c4Cl)n3)c2)cn1 ; Cn1cc(C(=O)Nc2cccc(-c3nc(Nc4ccc5[nH]ncc5c4Cl)no3)c2)cn1 ; O=C(Nc1cccc(-c2nc(Nc3ccc4[nH]ncc4c3Cl)no2)c1)c1ccccc1F ; Cn1cc(-c2nc3cc(-n4ccc(Nc5ccc6[nH]ncc6c5Cl)n4)ccc3o2)cn1]

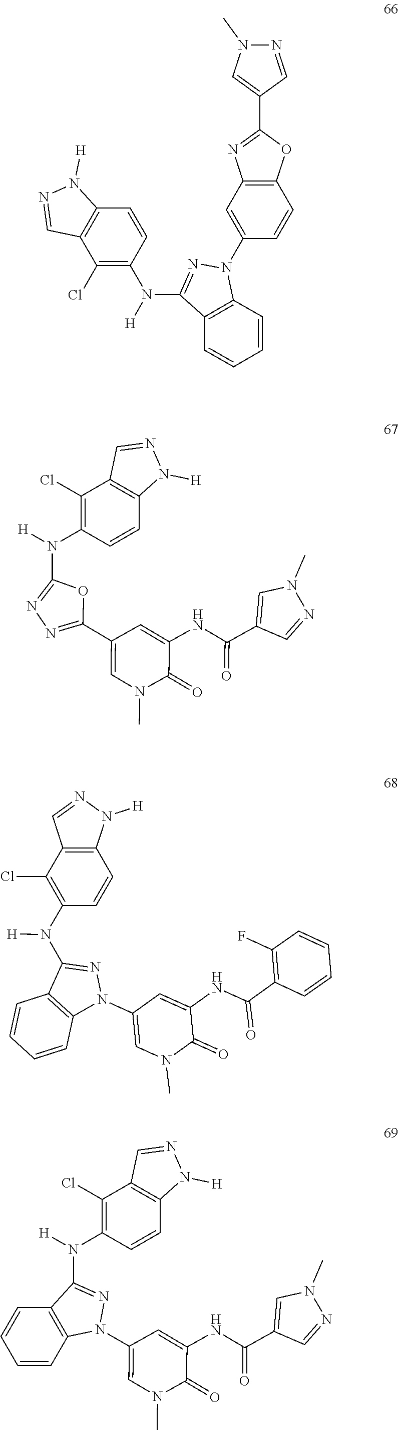 [SMILES: Cn1cc(-c2nc3cc(-n4nc(Nc5ccc6[nH]ncc6c5Cl)c5ccccc54)ccc3o2)cn1 ; Cn1cc(C(=O)Nc2cc(-c3nnc(Nc4ccc5[nH]ncc5c4Cl)o3)cn(C)c2=O)cn1 ; Cn1cc(-n2nc(Nc3ccc4[nH]ncc4c3Cl)c3ccccc32)cc(NC(=O)c2ccccc2F)c1=O ; Cn1cc(C(=O)Nc2cc(-n3nc(Nc4ccc5[nH]ncc5c4Cl)c4ccccc43)cn(C)c2=O)cn1]

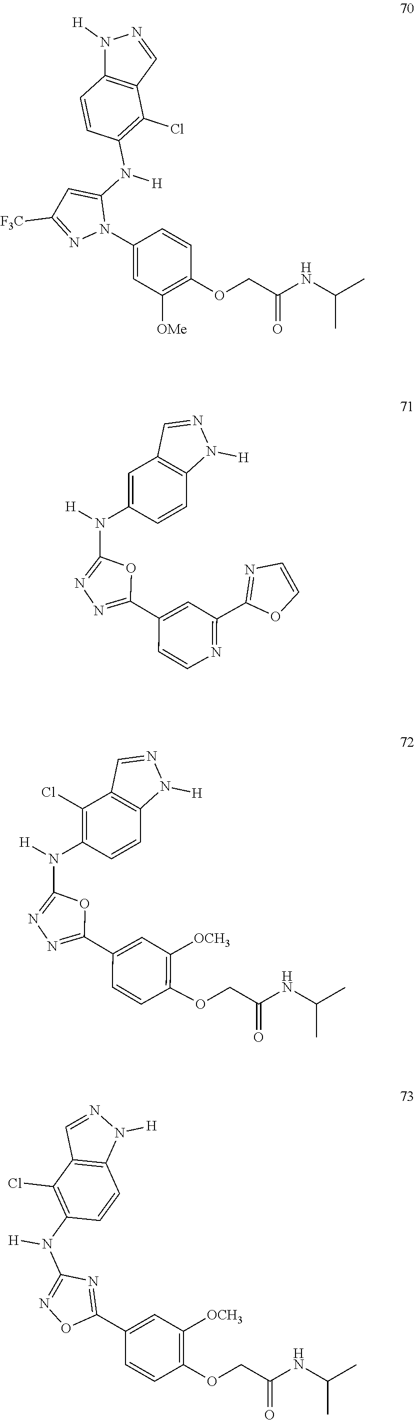 [SMILES: COc1cc(-n2nc(C(F)(F)F)cc2Nc2ccc3[nH]ncc3c2Cl)ccc1OCC(=O)NC(C)C ; c1cc(-c2nnc(Nc3ccc4[nH]ncc4c3)o2)cc(-c2ncco2)n1 ; COc1cc(-c2nnc(Nc3ccc4[nH]ncc4c3Cl)o2)ccc1OCC(=O)NC(C)C ; COc1cc(-c2nc(Nc3ccc4[nH]ncc4c3Cl)no2)ccc1OCC(=O)NC(C)C]

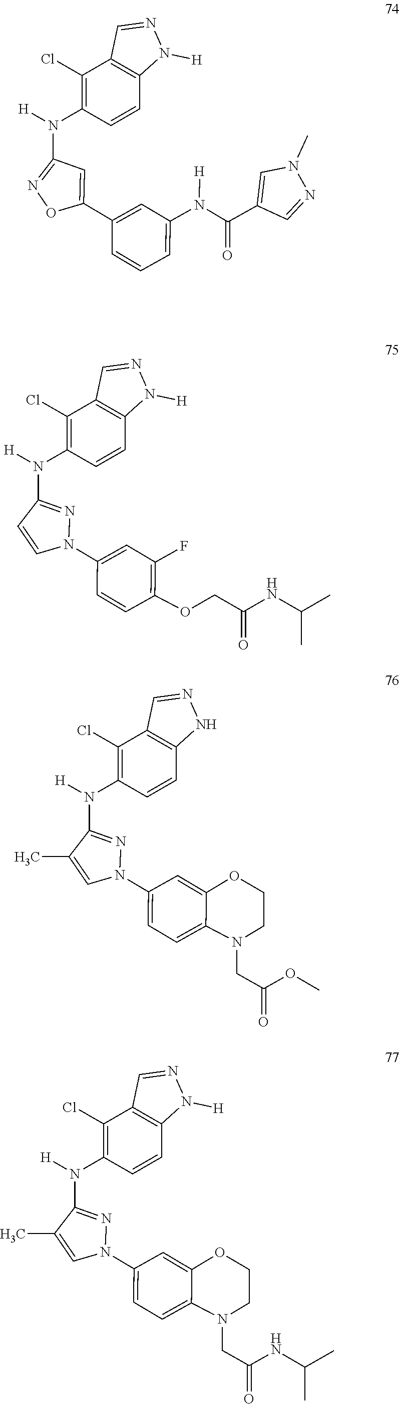

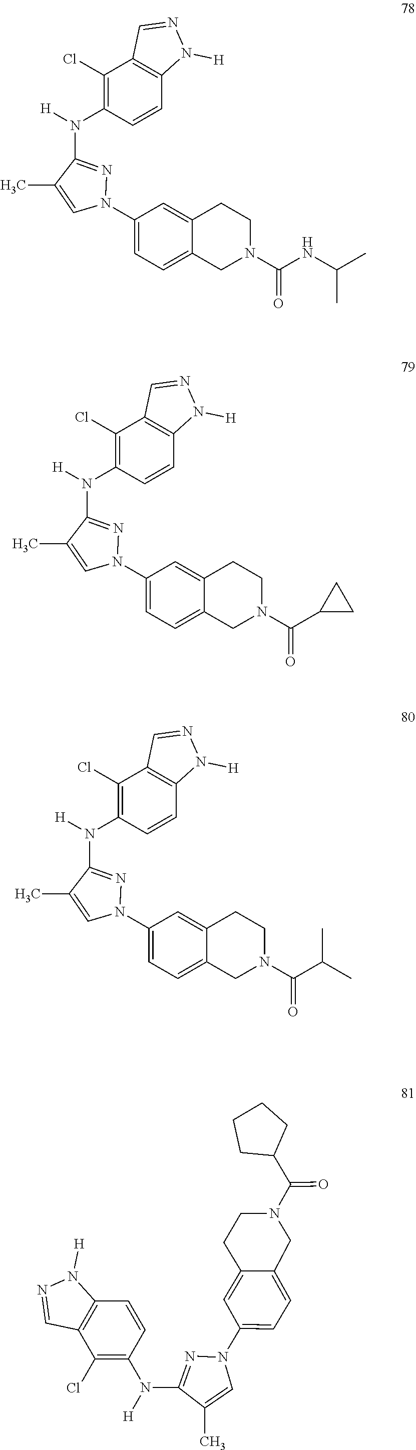

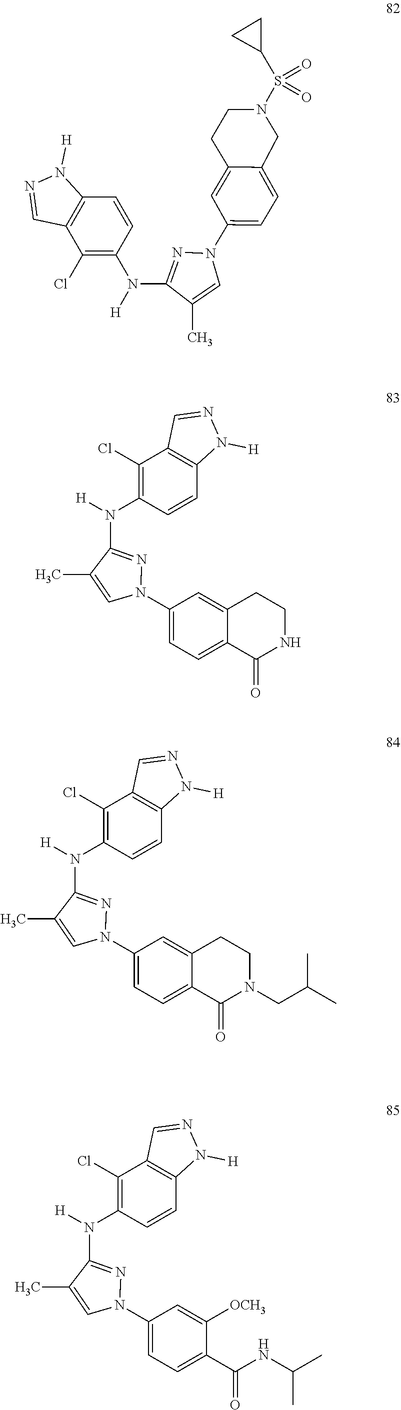

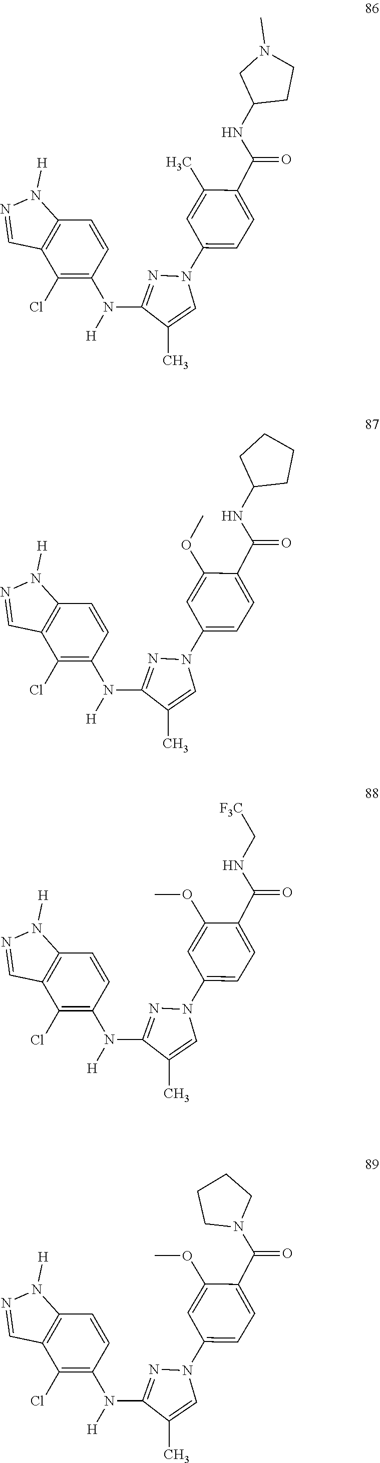 [SMILES: Cc1cc(-n2cc(C)c(Nc3ccc4[nH]ncc4c3Cl)n2)ccc1C(=O)NC1CCN(C)C1 ; COc1cc(-n2cc(C)c(Nc3ccc4[nH]ncc4c3Cl)n2)ccc1C(=O)NC1CCCC1 ; COc1cc(-n2cc(C)c(Nc3ccc4[nH]ncc4c3Cl)n2)ccc1C(=O)NCC(F)(F)F ; COc1cc(-n2cc(C)c(Nc3ccc4[nH]ncc4c3Cl)n2)ccc1C(=O)N1CCCC1]

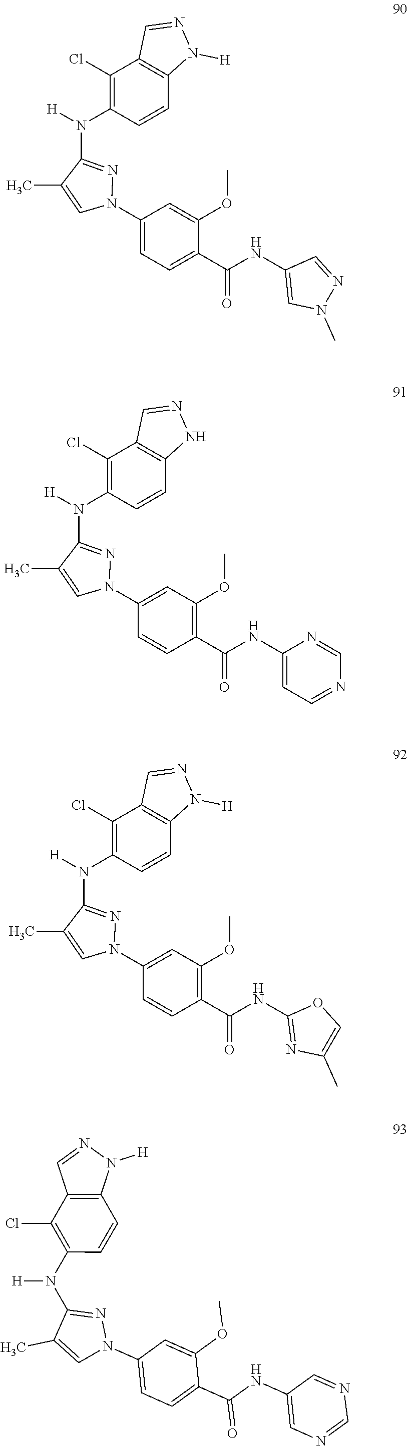 [SMILES: COc1cc(-n2cc(C)c(Nc3ccc4[nH]ncc4c3Cl)n2)ccc1C(=O)Nc1cnn(C)c1 ; COc1cc(-n2cc(C)c(Nc3ccc4[nH]ncc4c3Cl)n2)ccc1C(=O)Nc1ccncn1 ; COc1cc(-n2cc(C)c(Nc3ccc4[nH]ncc4c3Cl)n2)ccc1C(=O)Nc1nc(C)co1 ; COc1cc(-n2cc(C)c(Nc3ccc4[nH]ncc4c3Cl)n2)ccc1C(=O)Nc1cncnc1]

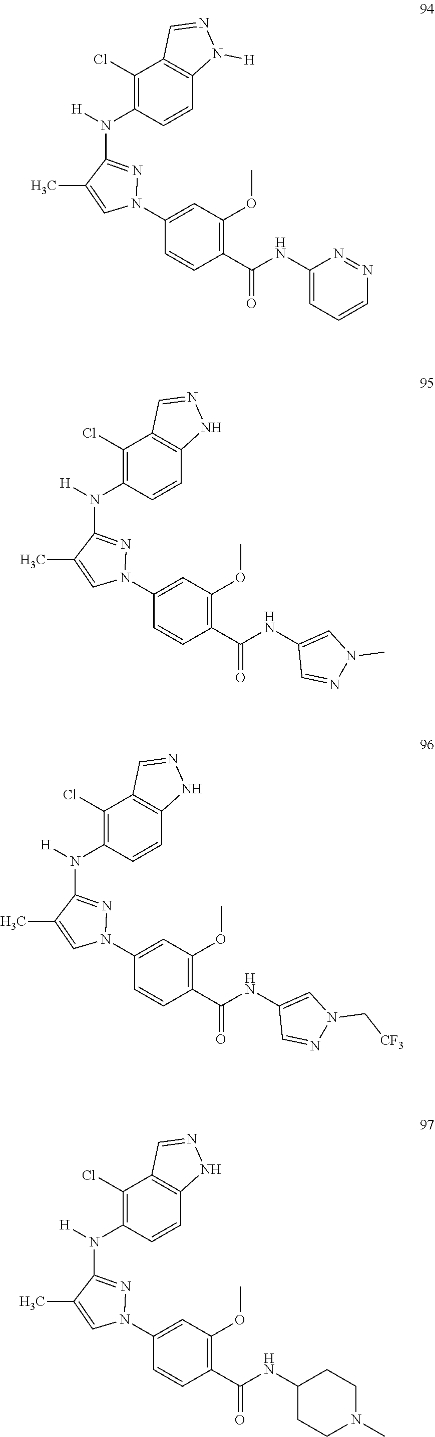

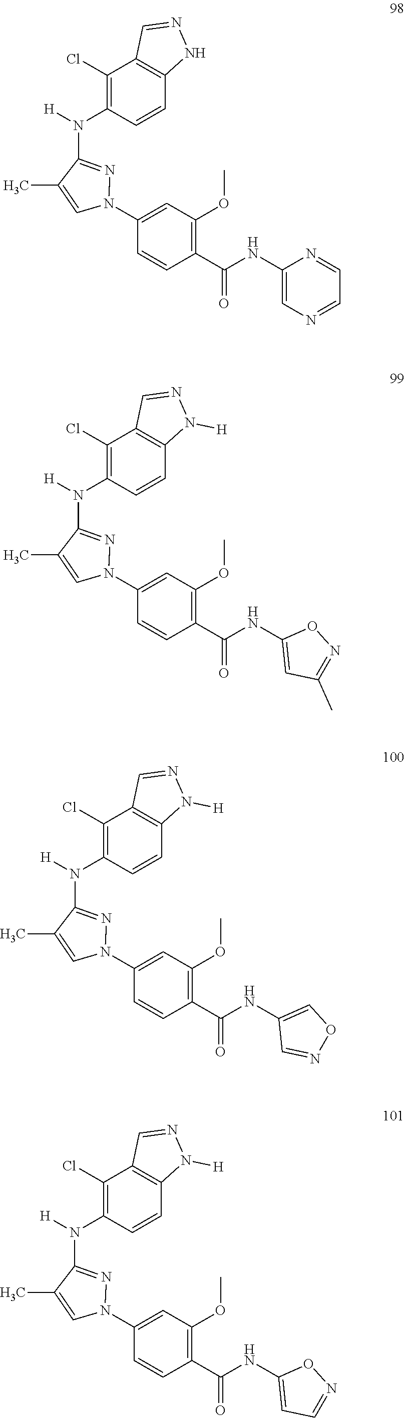 [SMILES: COc1cc(-n2cc(C)c(Nc3ccc4[nH]ncc4c3Cl)n2)ccc1C(=O)Nc1cnccn1 ; COc1cc(-n2cc(C)c(Nc3ccc4[nH]ncc4c3Cl)n2)ccc1C(=O)Nc1cc(C)no1 ; COc1cc(-n2cc(C)c(Nc3ccc4[nH]ncc4c3Cl)n2)ccc1C(=O)Nc1cnoc1 ; COc1cc(-n2cc(C)c(Nc3ccc4[nH]ncc4c3Cl)n2)ccc1C(=O)Nc1ccno1]

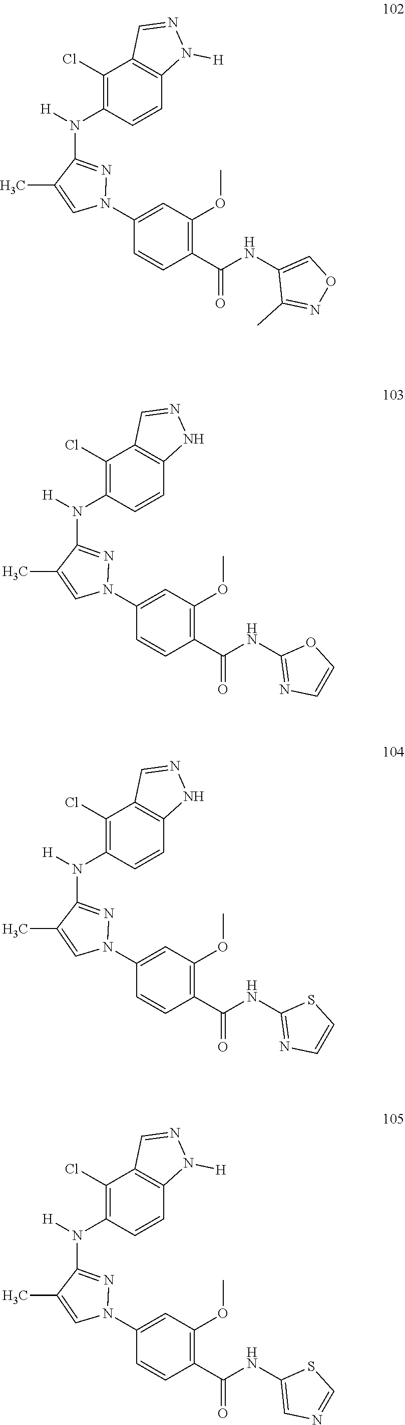 [SMILES: COc1cc(-n2cc(C)c(Nc3ccc4[nH]ncc4c3Cl)n2)ccc1C(=O)Nc1conc1C ; COc1cc(-n2cc(C)c(Nc3ccc4[nH]ncc4c3Cl)n2)ccc1C(=O)Nc1ncco1 ; COc1cc(-n2cc(C)c(Nc3ccc4[nH]ncc4c3Cl)n2)ccc1C(=O)Nc1nccs1 ; COc1cc(-n2cc(C)c(Nc3ccc4[nH]ncc4c3Cl)n2)ccc1C(=O)Nc1cncs1]

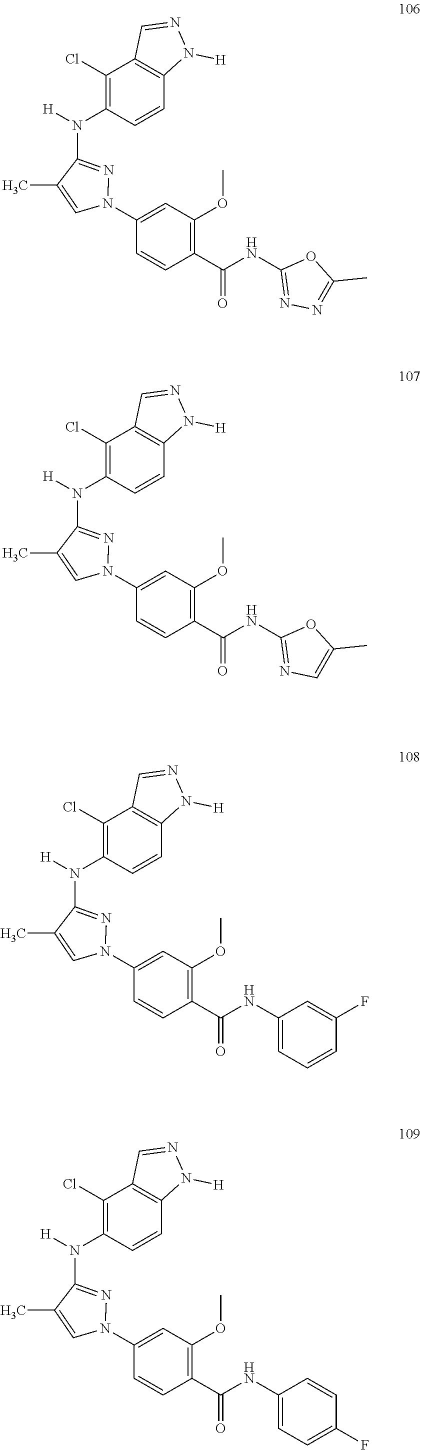

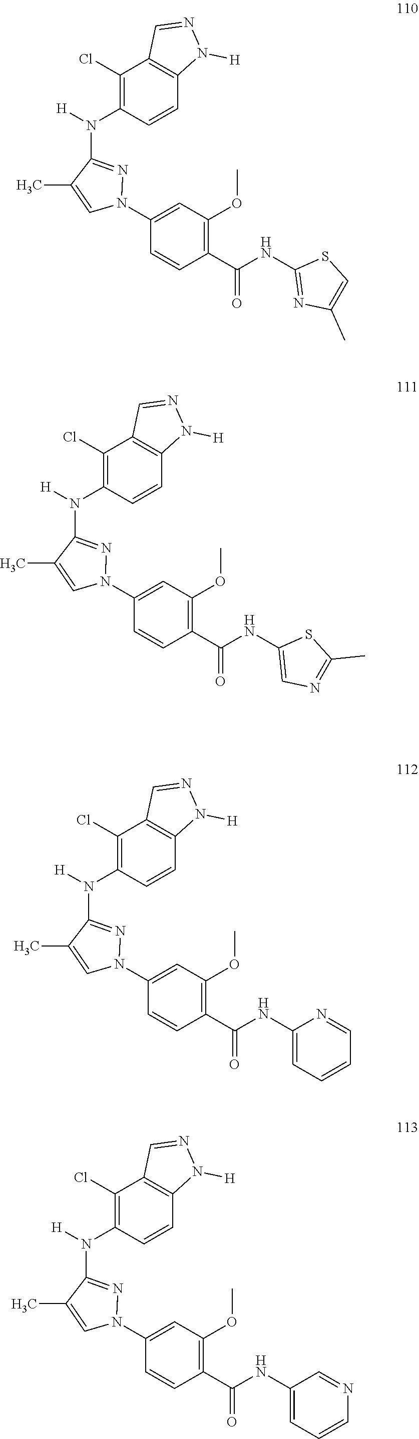

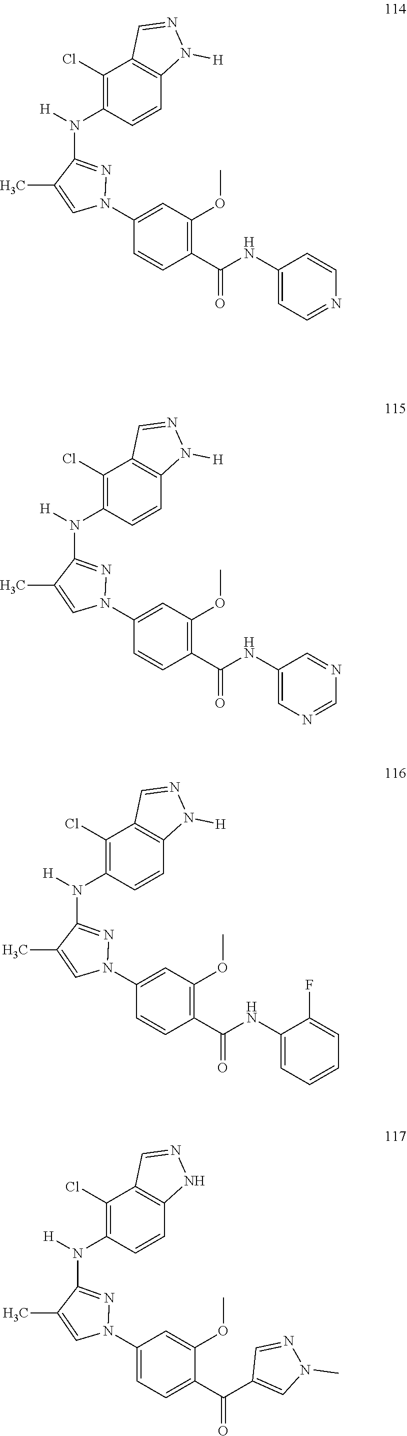

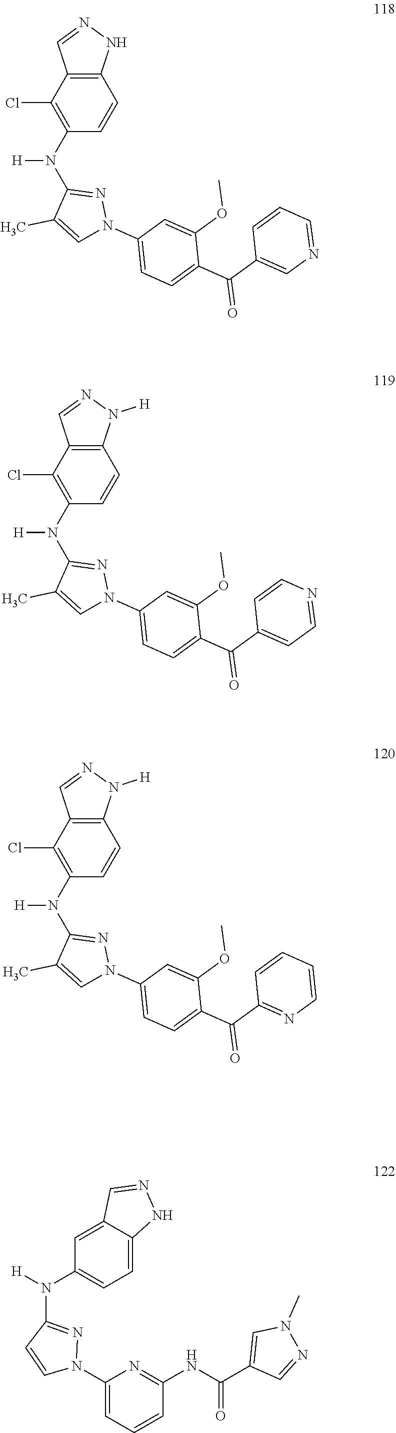 [SMILES: COc1cc(-n2cc(C)c(Nc3ccc4[nH]ncc4c3Cl)n2)ccc1C(=O)c1cccnc1 ; COc1cc(-n2cc(C)c(Nc3ccc4[nH]ncc4c3Cl)n2)ccc1C(=O)c1ccncc1 ; COc1cc(-n2cc(C)c(Nc3ccc4[nH]ncc4c3Cl)n2)ccc1C(=O)c1ccccn1 ; Cn1cc(C(=O)Nc2cccc(-n3ccc(Nc4ccc5[nH]ncc5c4)n3)n2)cn1]

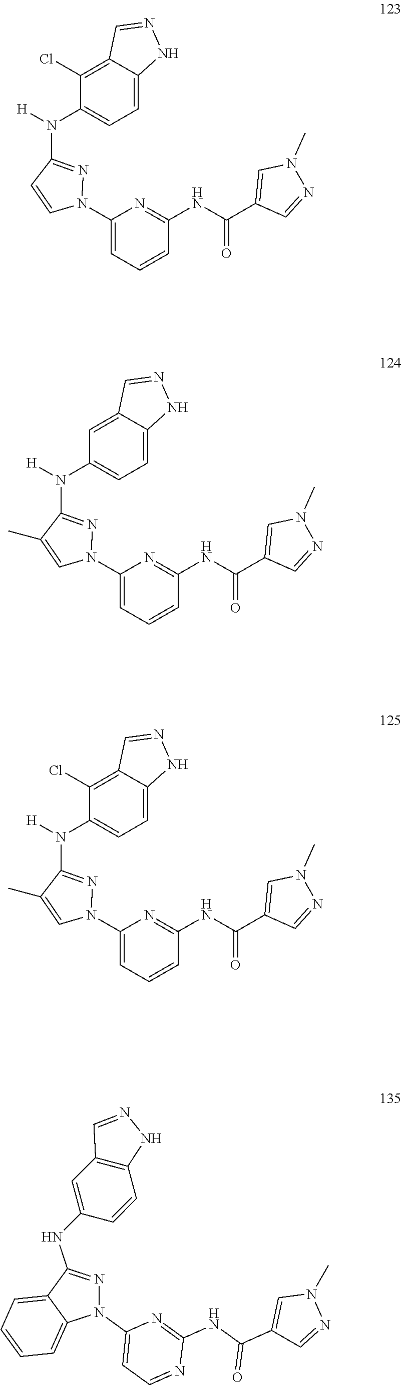 [SMILES: Cn1cc(C(=O)Nc2cccc(-n3ccc(Nc4ccc5[nH]ncc5c4Cl)n3)n2)cn1 ; Cc1cn(-c2cccc(NC(=O)c3cnn(C)c3)n2)nc1Nc1ccc2[nH]ncc2c1 ; Cc1cn(-c2cccc(NC(=O)c3cnn(C)c3)n2)nc1Nc1ccc2[nH]ncc2c1Cl ; Cn1cc(C(=O)Nc2nccc(-n3nc(Nc4ccc5[nH]ncc5c4)c4ccccc43)n2)cn1]

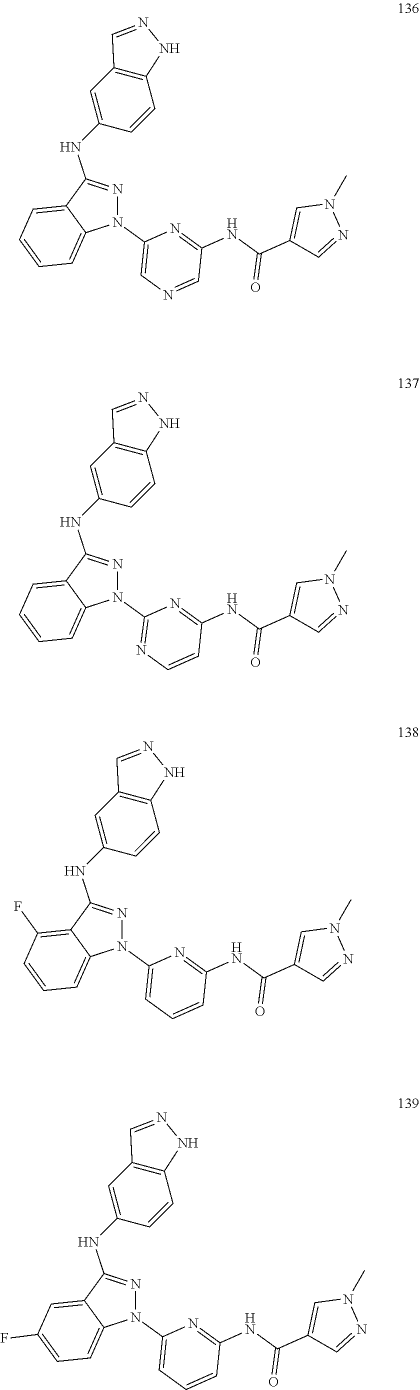 [SMILES: Cn1cc(C(=O)Nc2cncc(-n3nc(Nc4ccc5[nH]ncc5c4)c4ccccc43)n2)cn1 ; Cn1cc(C(=O)Nc2ccnc(-n3nc(Nc4ccc5[nH]ncc5c4)c4ccccc43)n2)cn1 ; Cn1cc(C(=O)Nc2cccc(-n3nc(Nc4ccc5[nH]ncc5c4)c4c(F)cccc43)n2)cn1 ; Cn1cc(C(=O)Nc2cccc(-n3nc(Nc4ccc5[nH]ncc5c4)c4cc(F)ccc43)n2)cn1]

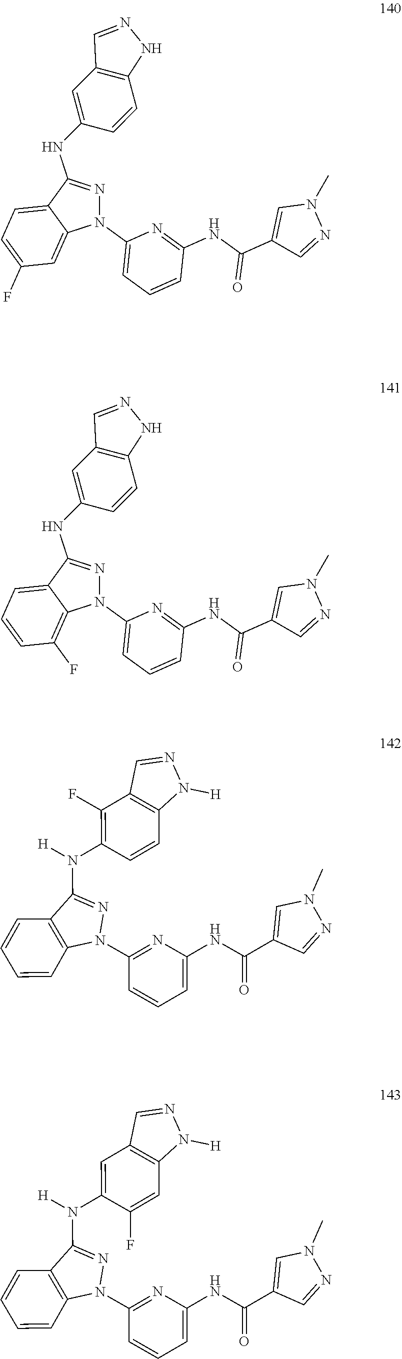 [SMILES: Cn1cc(C(=O)Nc2cccc(-n3nc(Nc4ccc5[nH]ncc5c4)c4ccc(F)cc43)n2)cn1 ; Cn1cc(C(=O)Nc2cccc(-n3nc(Nc4ccc5[nH]ncc5c4)c4cccc(F)c43)n2)cn1 ; Cn1cc(C(=O)Nc2cccc(-n3nc(Nc4ccc5[nH]ncc5c4F)c4ccccc43)n2)cn1 ; Cn1cc(C(=O)Nc2cccc(-n3nc(Nc4cc5cn[nH]c5cc4F)c4ccccc43)n2)cn1]

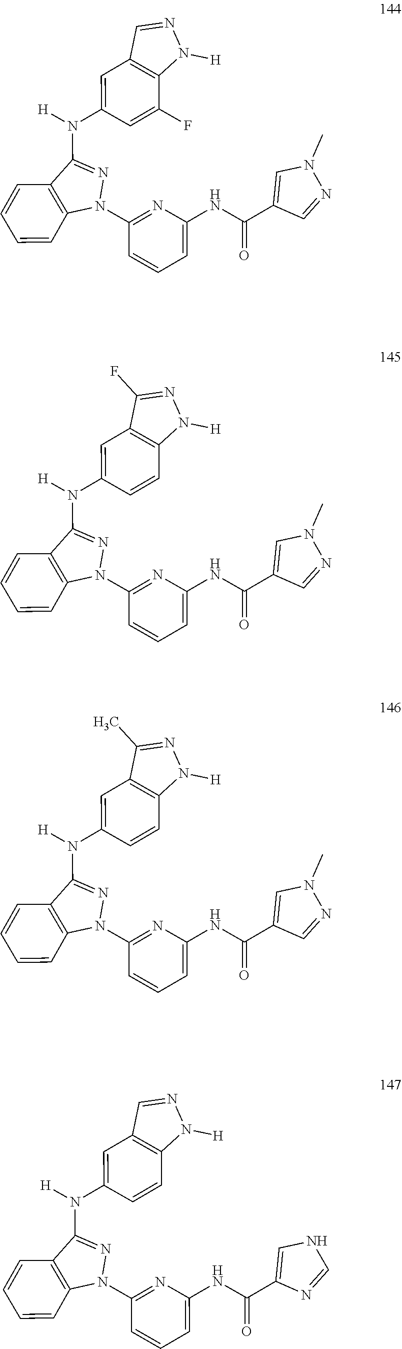 [SMILES: Cn1cc(C(=O)Nc2cccc(-n3nc(Nc4cc(F)c5[nH]ncc5c4)c4ccccc43)n2)cn1 ; Cn1cc(C(=O)Nc2cccc(-n3nc(Nc4ccc5[nH]nc(F)c5c4)c4ccccc43)n2)cn1 ; Cc1n[nH]c2ccc(Nc3nn(-c4cccc(NC(=O)c5cnn(C)c5)n4)c4ccccc34)cc12 ; O=C(Nc1cccc(-n2nc(Nc3ccc4[nH]ncc4c3)c3ccccc32)n1)c1c[nH]cn1]

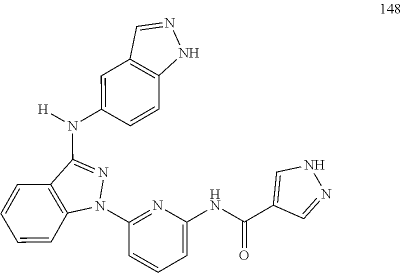 [SMILES: O=C(Nc1cccc(-n2nc(Nc3ccc4[nH]ncc4c3)c3ccccc32)n1)c1cn[nH]c1]

In certain embodiments, the compound of Formula (I) is one of the following compounds, or a pharmaceutically acceptable salt, co-crystal, tautomer, stereoisomer, solvate, hydrate, polymorph, isotopically enriched compound, or prodrug thereof:

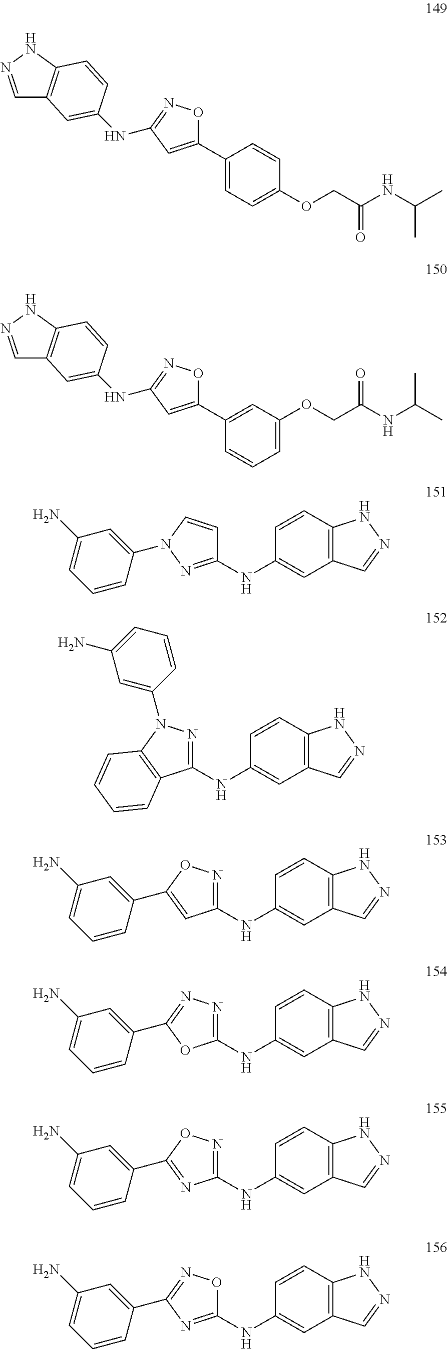

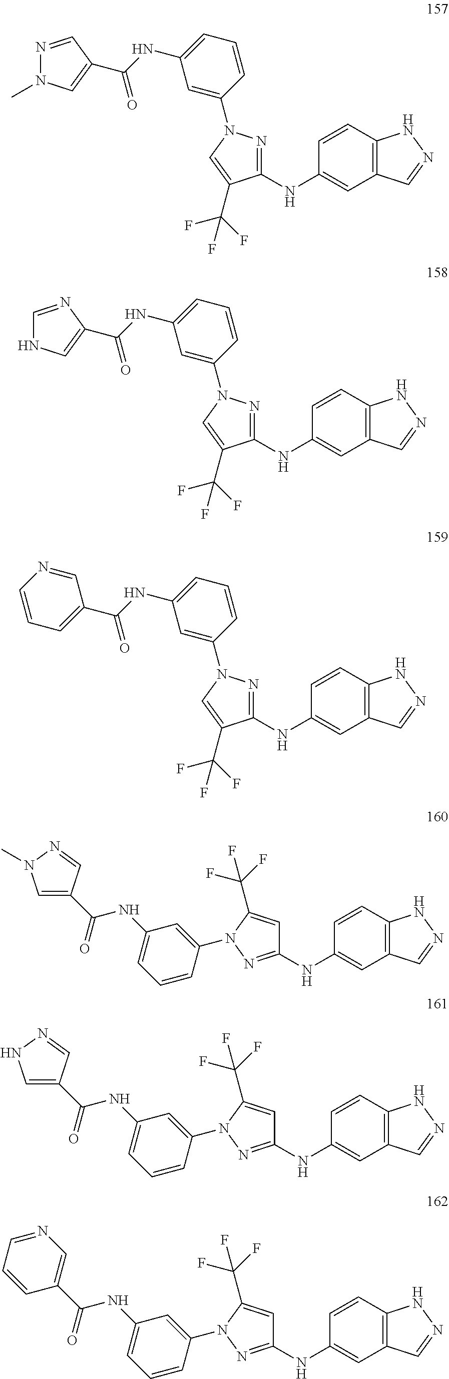 [SMILES: Cn1cc(C(=O)Nc2cccc(-n3cc(C(F)(F)F)c(Nc4ccc5[nH]ncc5c4)n3)c2)cn1 ; O=C(Nc1cccc(-n2cc(C(F)(F)F)c(Nc3ccc4[nH]ncc4c3)n2)c1)c1c[nH]cn1 ; O=C(Nc1cccc(-n2cc(C(F)(F)F)c(Nc3ccc4[nH]ncc4c3)n2)c1)c1cccnc1 ; Cn1cc(C(=O)Nc2cccc(-n3nc(Nc4ccc5[nH]ncc5c4)cc3C(F)(F)F)c2)cn1 ; O=C(Nc1cccc(-n2nc(Nc3ccc4[nH]ncc4c3)cc2C(F)(F)F)c1)c1cn[nH]c1 ; O=C(Nc1cccc(-n2nc(Nc3ccc4[nH]ncc4c3)cc2C(F)(F)F)c1)c1cccnc1]

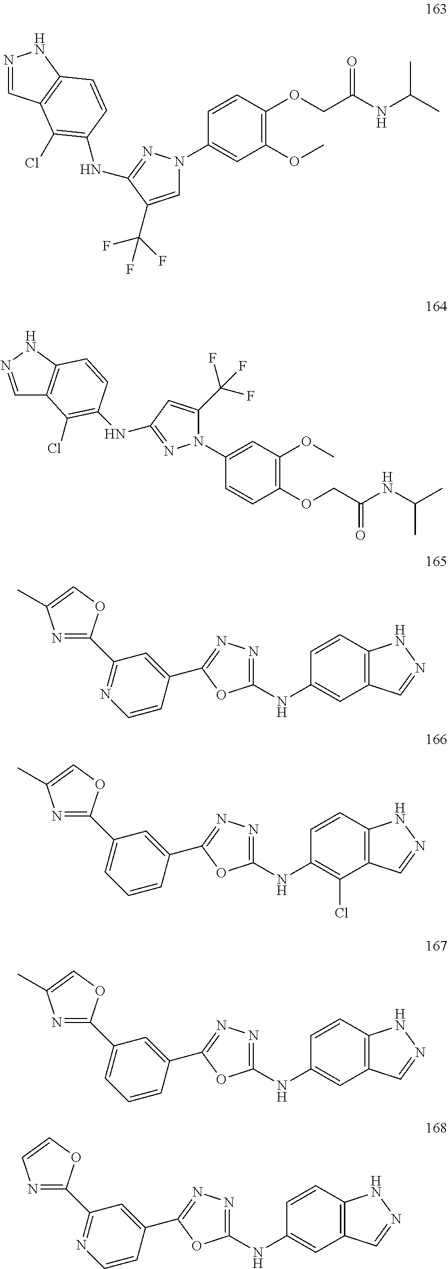

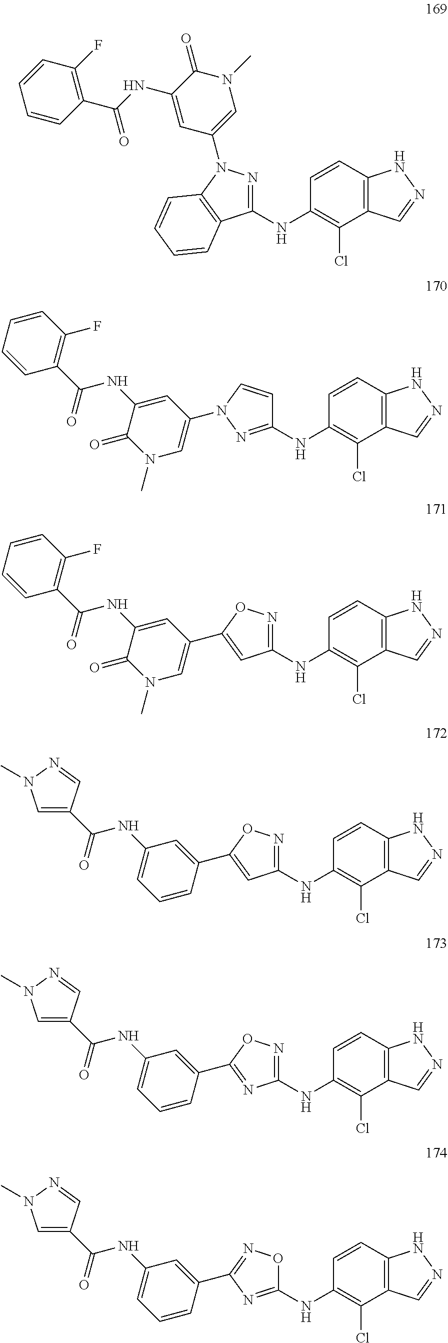 [SMILES: Cn1cc(-n2nc(Nc3ccc4[nH]ncc4c3Cl)c3ccccc32)cc(NC(=O)c2ccccc2F)c1=O ; Cn1cc(-n2ccc(Nc3ccc4[nH]ncc4c3Cl)n2)cc(NC(=O)c2ccccc2F)c1=O ; Cn1cc(-c2cc(Nc3ccc4[nH]ncc4c3Cl)no2)cc(NC(=O)c2ccccc2F)c1=O ; Cn1cc(C(=O)Nc2cccc(-c3cc(Nc4ccc5[nH]ncc5c4Cl)no3)c2)cn1 ; Cn1cc(C(=O)Nc2cccc(-c3nc(Nc4ccc5[nH]ncc5c4Cl)no3)c2)cn1 ; Cn1cc(C(=O)Nc2cccc(-c3noc(Nc4ccc5[nH]ncc5c4Cl)n3)c2)cn1]

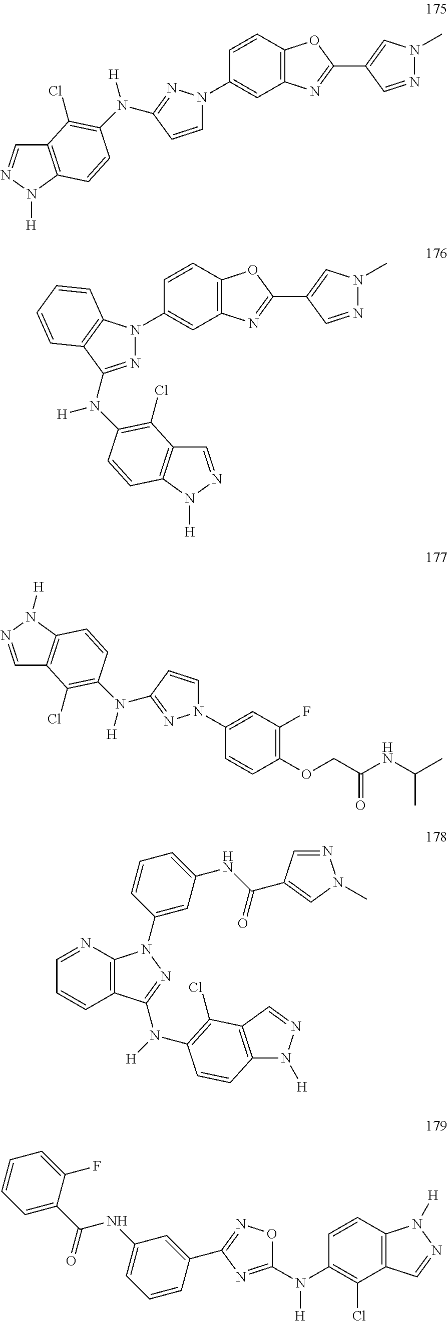 [SMILES: Cn1cc(-c2nc3cc(-n4ccc(Nc5ccc6[nH]ncc6c5Cl)n4)ccc3o2)cn1 ; Cn1cc(-c2nc3cc(-n4nc(Nc5ccc6[nH]ncc6c5Cl)c5ccccc54)ccc3o2)cn1 ; CC(C)NC(=O)COc1ccc(-n2ccc(Nc3ccc4[nH]ncc4c3Cl)n2)cc1F ; Cn1cc(C(=O)Nc2cccc(-n3nc(Nc4ccc5[nH]ncc5c4Cl)c4cccnc43)c2)cn1 ; O=C(Nc1cccc(-c2noc(Nc3ccc4[nH]ncc4c3Cl)n2)c1)c1ccccc1F]

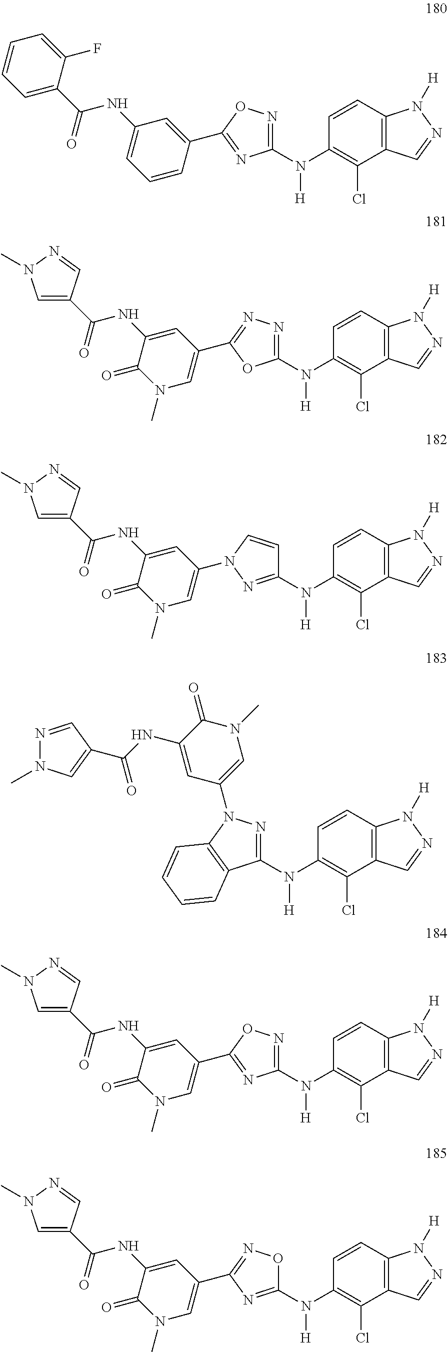 [SMILES: O=C(Nc1cccc(-c2nc(Nc3ccc4[nH]ncc4c3Cl)no2)c1)c1ccccc1F ; Cn1cc(C(=O)Nc2cc(-c3nnc(Nc4ccc5[nH]ncc5c4Cl)o3)cn(C)c2=O)cn1 ; Cn1cc(C(=O)Nc2cc(-n3ccc(Nc4ccc5[nH]ncc5c4Cl)n3)cn(C)c2=O)cn1 ; Cn1cc(C(=O)Nc2cc(-n3nc(Nc4ccc5[nH]ncc5c4Cl)c4ccccc43)cn(C)c2=O)cn1 ; Cn1cc(C(=O)Nc2cc(-c3nc(Nc4ccc5[nH]ncc5c4Cl)no3)cn(C)c2=O)cn1 ; Cn1cc(C(=O)Nc2cc(-c3noc(Nc4ccc5[nH]ncc5c4Cl)n3)cn(C)c2=O)cn1]

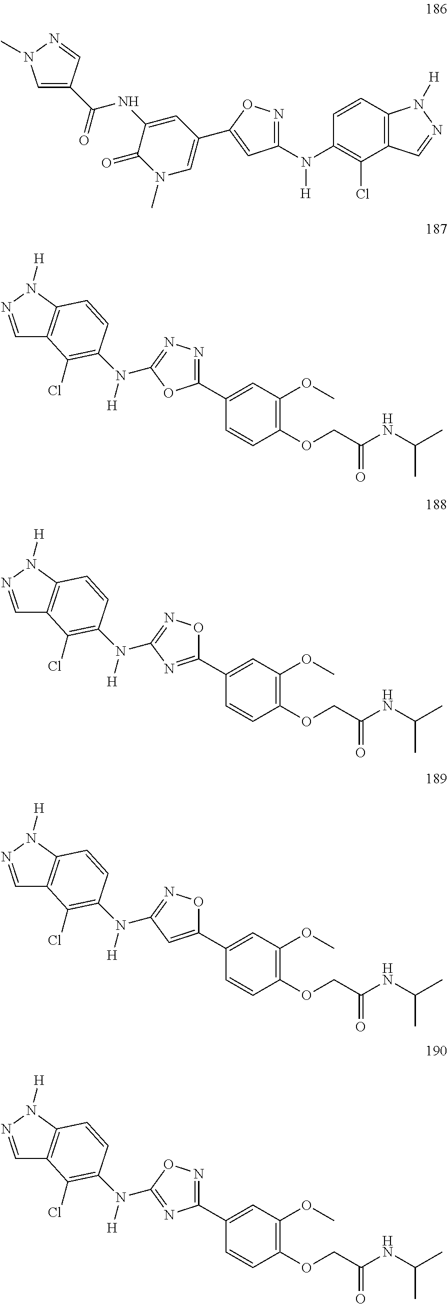 [SMILES: Cn1cc(C(=O)Nc2cc(-c3cc(Nc4ccc5[nH]ncc5c4Cl)no3)cn(C)c2=O)cn1 ; COc1cc(-c2nnc(Nc3ccc4[nH]ncc4c3Cl)o2)ccc1OCC(=O)NC(C)C ; COc1cc(-c2nc(Nc3ccc4[nH]ncc4c3Cl)no2)ccc1OCC(=O)NC(C)C ; COc1cc(-c2cc(Nc3ccc4[nH]ncc4c3Cl)no2)ccc1OCC(=O)NC(C)C ; COc1cc(-c2noc(Nc3ccc4[nH]ncc4c3Cl)n2)ccc1OCC(=O)NC(C)C]

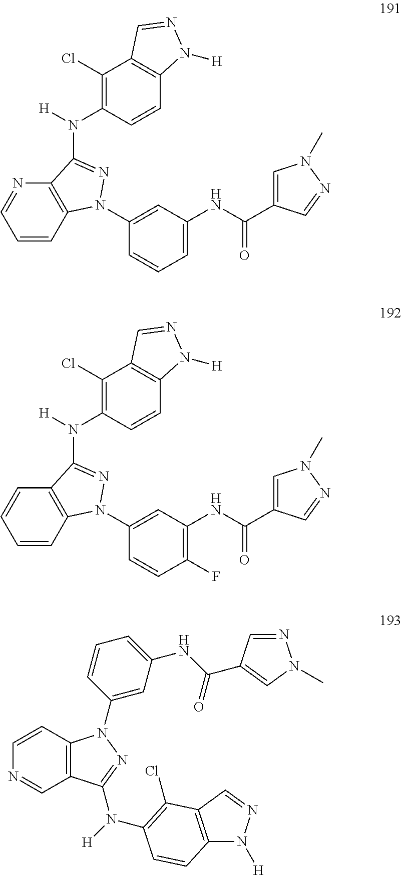 [SMILES: Cn1cc(C(=O)Nc2cccc(-n3nc(Nc4ccc5[nH]ncc5c4Cl)c4ncccc43)c2)cn1 ; Cn1cc(C(=O)Nc2cc(-n3nc(Nc4ccc5[nH]ncc5c4Cl)c4ccccc43)ccc2F)cn1 ; Cn1cc(C(=O)Nc2cccc(-n3nc(Nc4ccc5[nH]ncc5c4Cl)c4cnccc43)c2)cn1]

In certain embodiments, the compound of Formula (I) is one of the following compounds, or a pharmaceutically acceptable salt, co-crystal, tautomer, stereoisomer, solvate, hydrate, polymorph, isotopically enriched compound, or prodrug thereof:

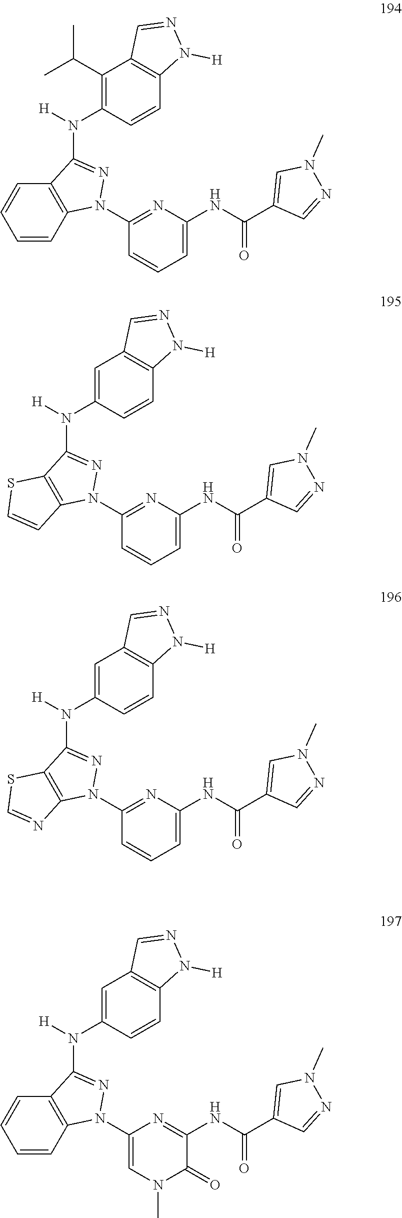

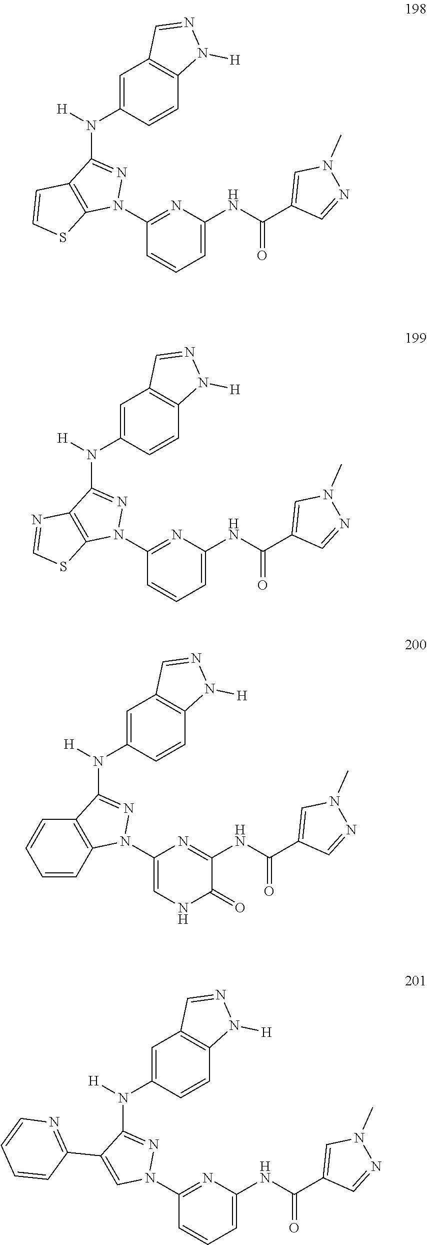 [SMILES: Cn1cc(C(=O)Nc2cccc(-n3nc(Nc4ccc5[nH]ncc5c4)c4ccsc43)n2)cn1 ; Cn1cc(C(=O)Nc2cccc(-n3nc(Nc4ccc5[nH]ncc5c4)c4ncsc43)n2)cn1 ; Cn1cc(C(=O)Nc2nc(-n3nc(Nc4ccc5[nH]ncc5c4)c4ccccc43)c[nH]c2=O)cn1 ; Cn1cc(C(=O)Nc2cccc(-n3cc(-c4ccccn4)c(Nc4ccc5[nH]ncc5c4)n3)n2)cn1]

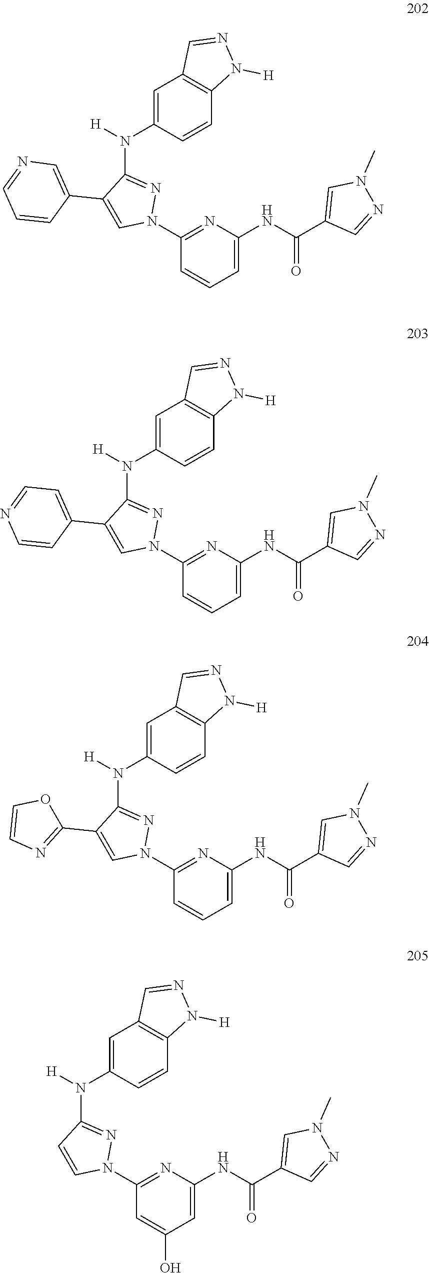 [SMILES: Cn1cc(C(=O)Nc2cccc(-n3cc(-c4cccnc4)c(Nc4ccc5[nH]ncc5c4)n3)n2)cn1 ; Cn1cc(C(=O)Nc2cccc(-n3cc(-c4ccncc4)c(Nc4ccc5[nH]ncc5c4)n3)n2)cn1 ; Cn1cc(C(=O)Nc2cccc(-n3cc(-c4ncco4)c(Nc4ccc5[nH]ncc5c4)n3)n2)cn1 ; Cn1cc(C(=O)Nc2cc(O)cc(-n3ccc(Nc4ccc5[nH]ncc5c4)n3)n2)cn1]

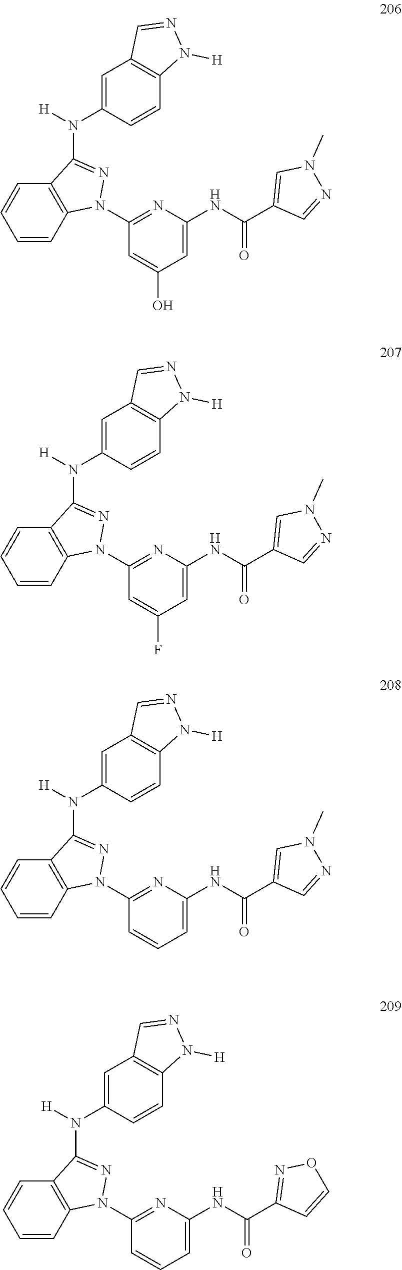 [SMILES: Cn1cc(C(=O)Nc2cc(O)cc(-n3nc(Nc4ccc5[nH]ncc5c4)c4ccccc43)n2)cn1 ; Cn1cc(C(=O)Nc2cc(F)cc(-n3nc(Nc4ccc5[nH]ncc5c4)c4ccccc43)n2)cn1 ; Cn1cc(C(=O)Nc2cccc(-n3nc(Nc4ccc5[nH]ncc5c4)c4ccccc43)n2)cn1 ; O=C(Nc1cccc(-n2nc(Nc3ccc4[nH]ncc4c3)c3ccccc32)n1)c1ccon1]

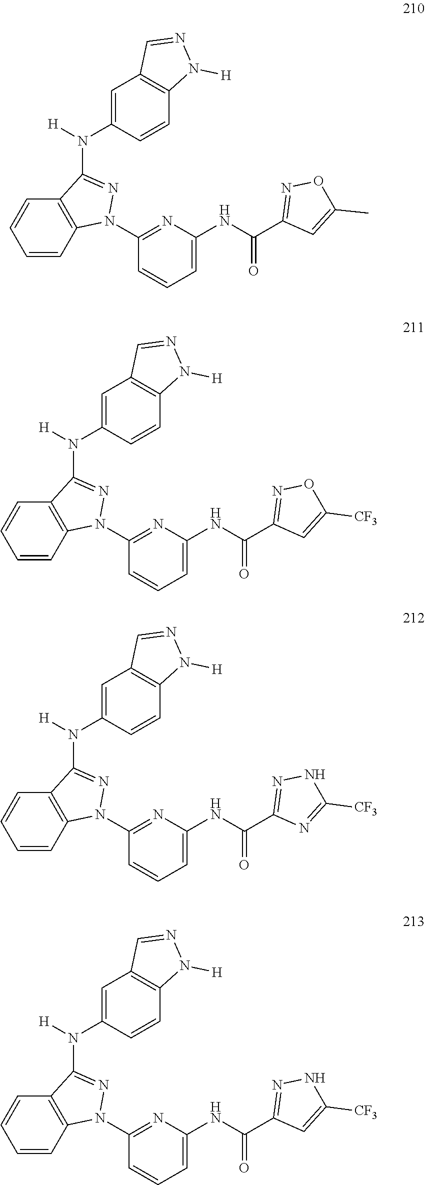

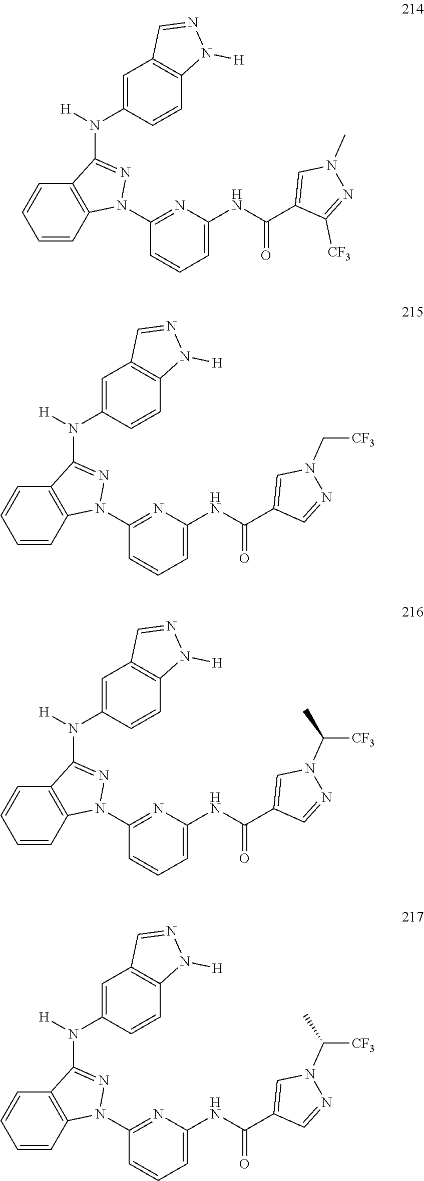 [SMILES: Cn1cc(C(=O)Nc2cccc(-n3nc(Nc4ccc5[nH]ncc5c4)c4ccccc43)n2)c(C(F)(F)F)n1 ; O=C(Nc1cccc(-n2nc(Nc3ccc4[nH]ncc4c3)c3ccccc32)n1)c1cnn(CC(F)(F)F)c1 ; C[C@H](n1cc(C(=O)Nc2cccc(-n3nc(Nc4ccc5[nH]ncc5c4)c4ccccc43)n2)cn1)C(F)(F)F ; C[C@@H](n1cc(C(=O)Nc2cccc(-n3nc(Nc4ccc5[nH]ncc5c4)c4ccccc43)n2)cn1)C(F)(F)F]

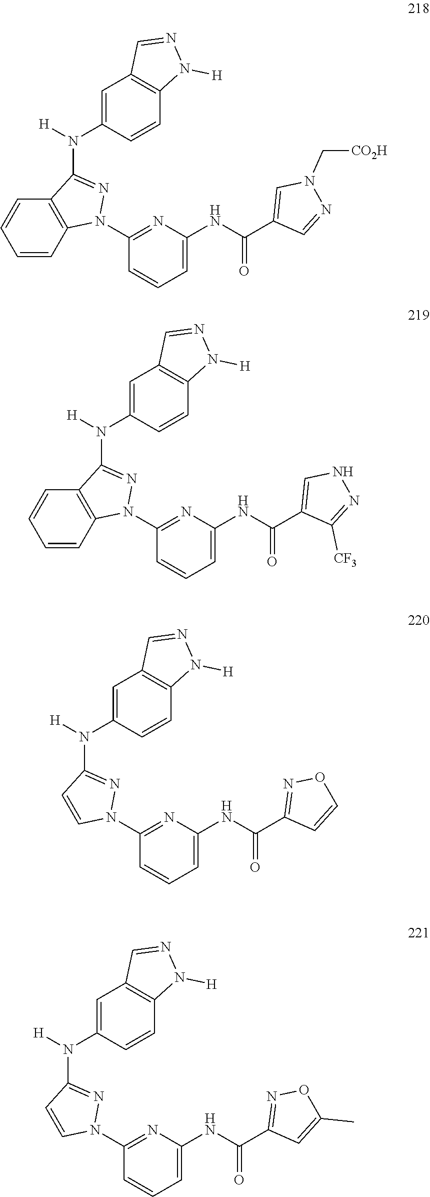

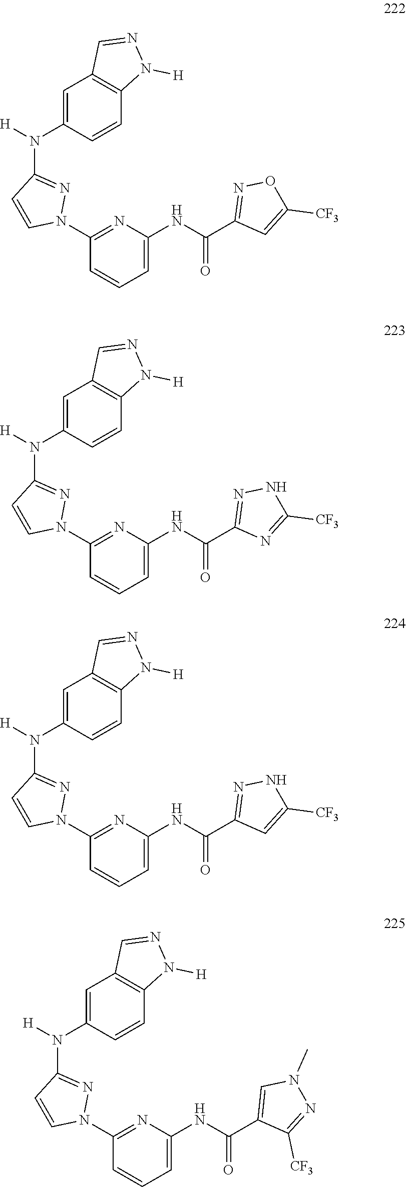

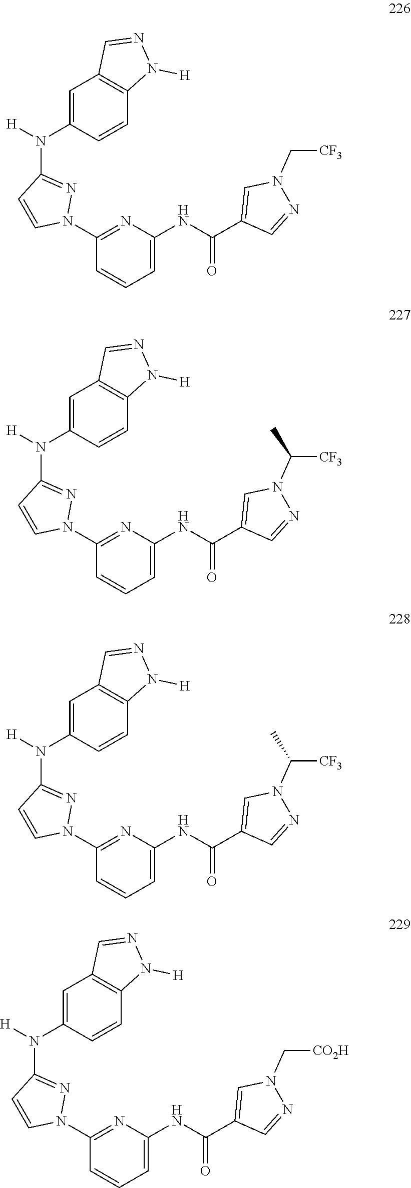

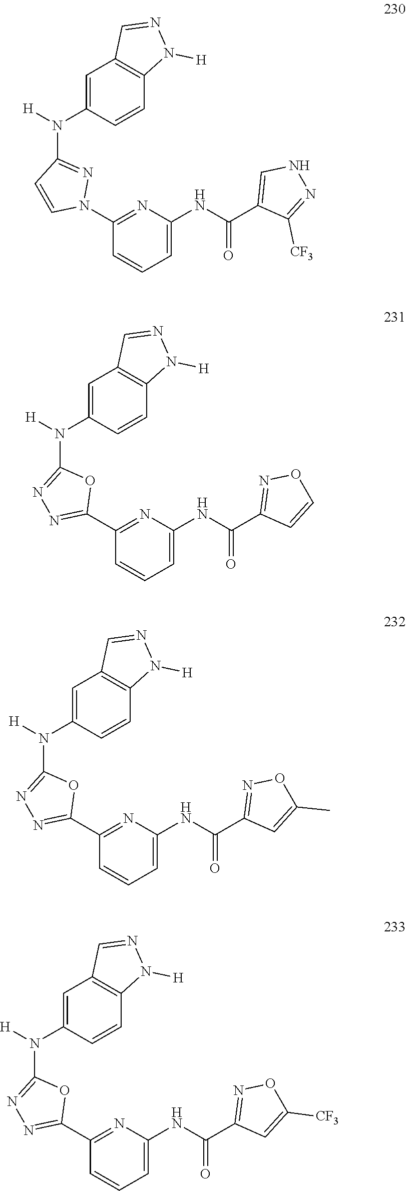 [SMILES: O=C(Nc1cccc(-n2ccc(Nc3ccc4[nH]ncc4c3)n2)n1)c1c[nH]nc1C(F)(F)F ; O=C(Nc1cccc(-c2nnc(Nc3ccc4[nH]ncc4c3)o2)n1)c1ccon1 ; Cc1cc(C(=O)Nc2cccc(-c3nnc(Nc4ccc5[nH]ncc5c4)o3)n2)no1 ; O=C(Nc1cccc(-c2nnc(Nc3ccc4[nH]ncc4c3)o2)n1)c1cc(C(F)(F)F)on1]

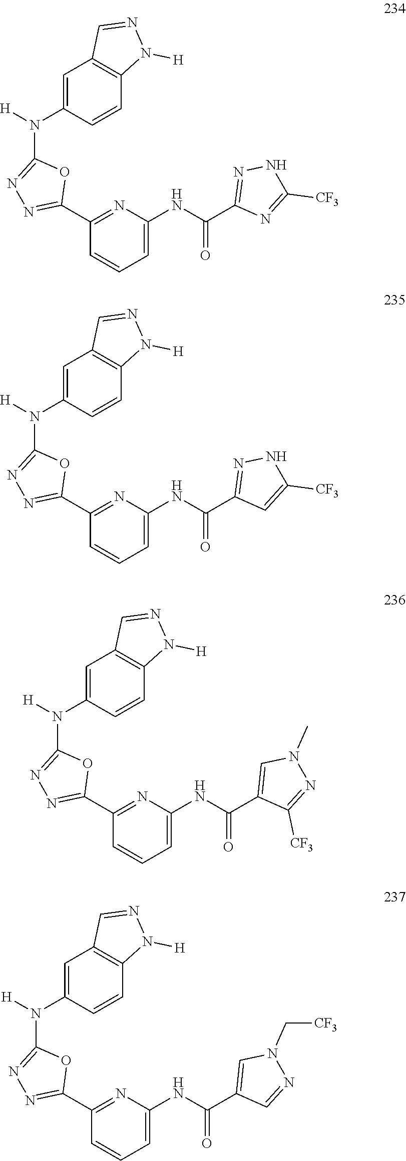

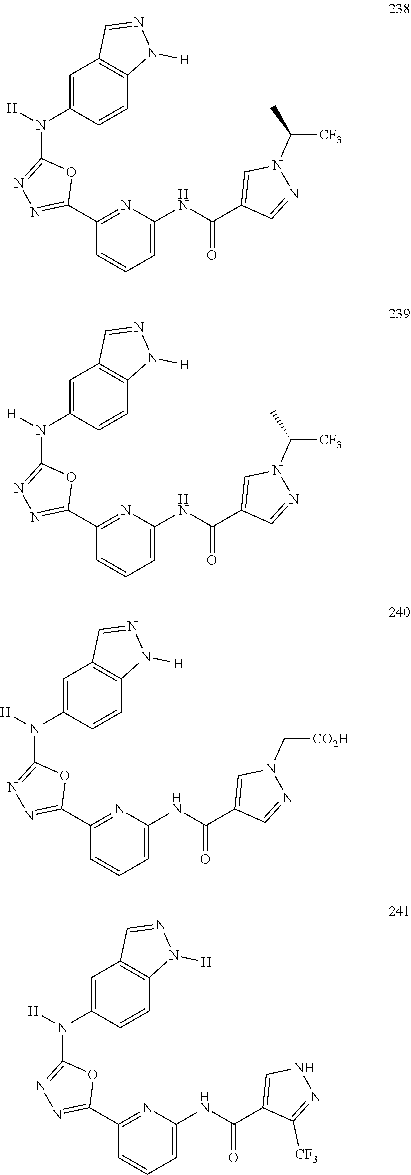 [SMILES: C[C@H](n1cc(C(=O)Nc2cccc(-c3nnc(Nc4ccc5[nH]ncc5c4)o3)n2)cn1)C(F)(F)F ; C[C@@H](n1cc(C(=O)Nc2cccc(-c3nnc(Nc4ccc5[nH]ncc5c4)o3)n2)cn1)C(F)(F)F ; O=C(O)Cn1cc(C(=O)Nc2cccc(-c3nnc(Nc4ccc5[nH]ncc5c4)o3)n2)cn1 ; O=C(Nc1cccc(-c2nnc(Nc3ccc4[nH]ncc4c3)o2)n1)c1c[nH]nc1C(F)(F)F]

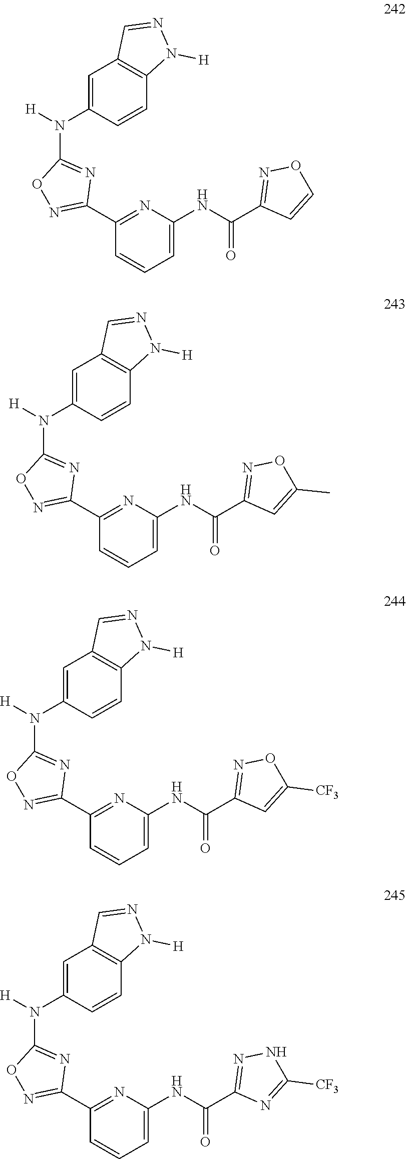

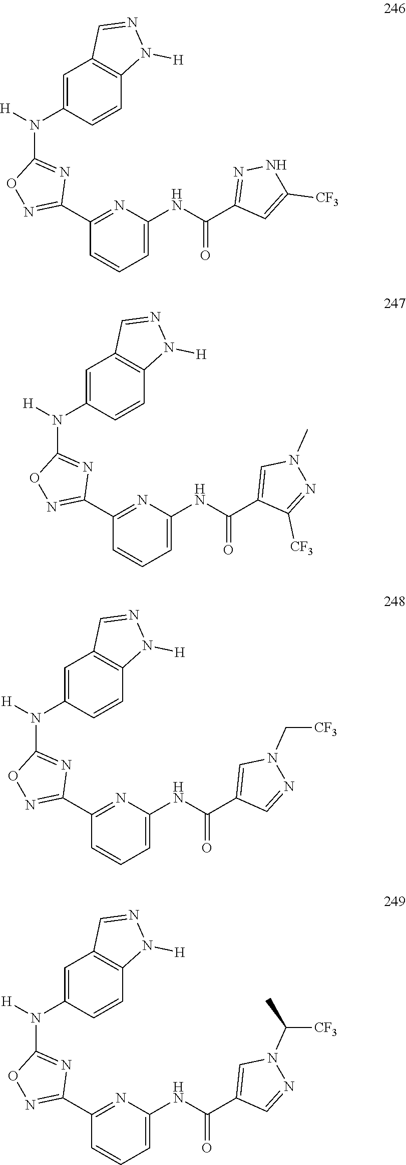 [SMILES: O=C(Nc1cccc(-c2noc(Nc3ccc4[nH]ncc4c3)n2)n1)c1cc(C(F)(F)F)[nH]n1 ; Cn1cc(C(=O)Nc2cccc(-c3noc(Nc4ccc5[nH]ncc5c4)n3)n2)c(C(F)(F)F)n1 ; O=C(Nc1cccc(-c2noc(Nc3ccc4[nH]ncc4c3)n2)n1)c1cnn(CC(F)(F)F)c1 ; C[C@H](n1cc(C(=O)Nc2cccc(-c3noc(Nc4ccc5[nH]ncc5c4)n3)n2)cn1)C(F)(F)F]

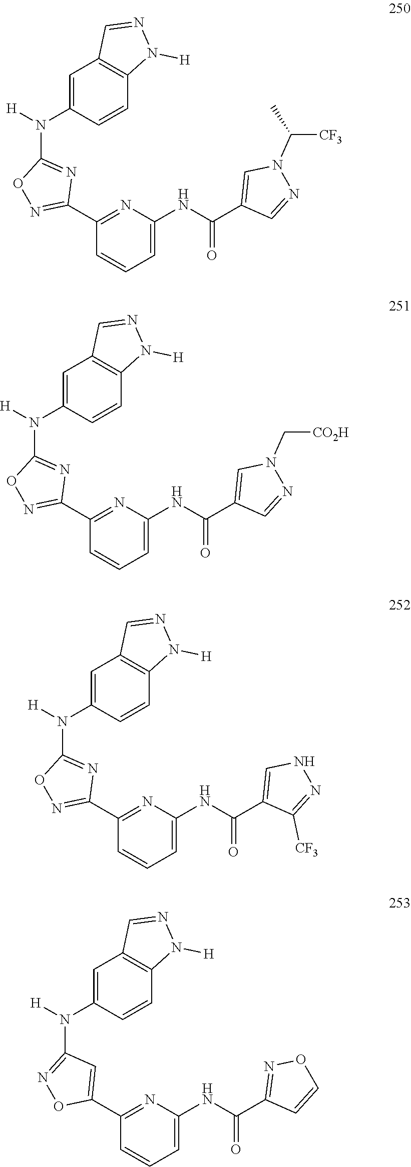 [SMILES: C[C@@H](n1cc(C(=O)Nc2cccc(-c3noc(Nc4ccc5[nH]ncc5c4)n3)n2)cn1)C(F)(F)F ; O=C(O)Cn1cc(C(=O)Nc2cccc(-c3noc(Nc4ccc5[nH]ncc5c4)n3)n2)cn1 ; O=C(Nc1cccc(-c2noc(Nc3ccc4[nH]ncc4c3)n2)n1)c1c[nH]nc1C(F)(F)F ; O=C(Nc1cccc(-c2cc(Nc3ccc4[nH]ncc4c3)no2)n1)c1ccon1]

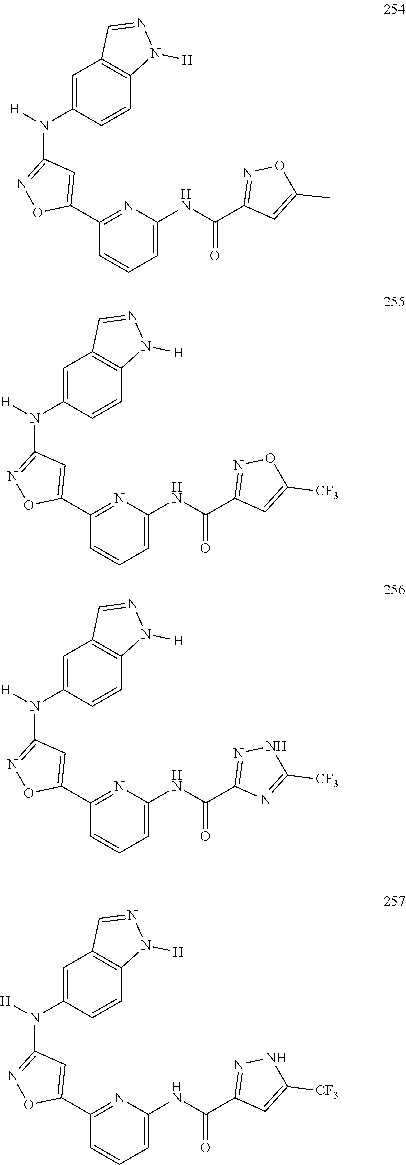

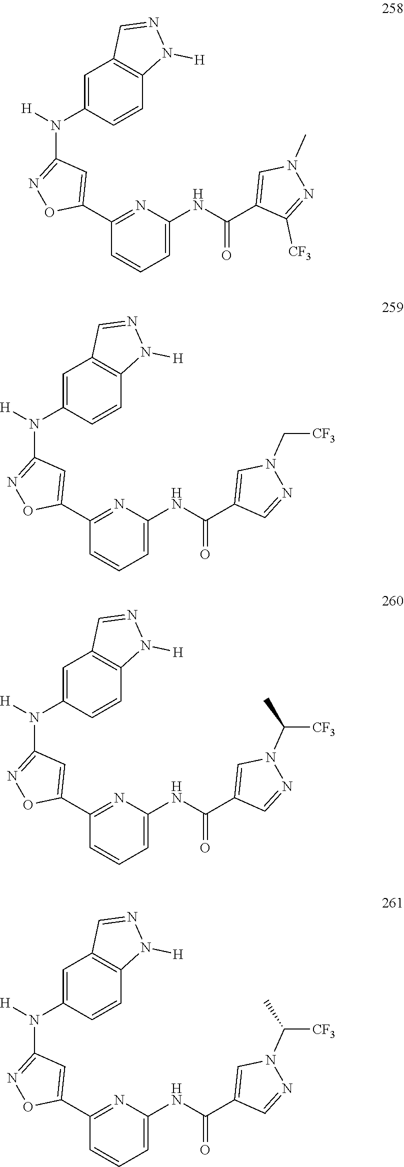

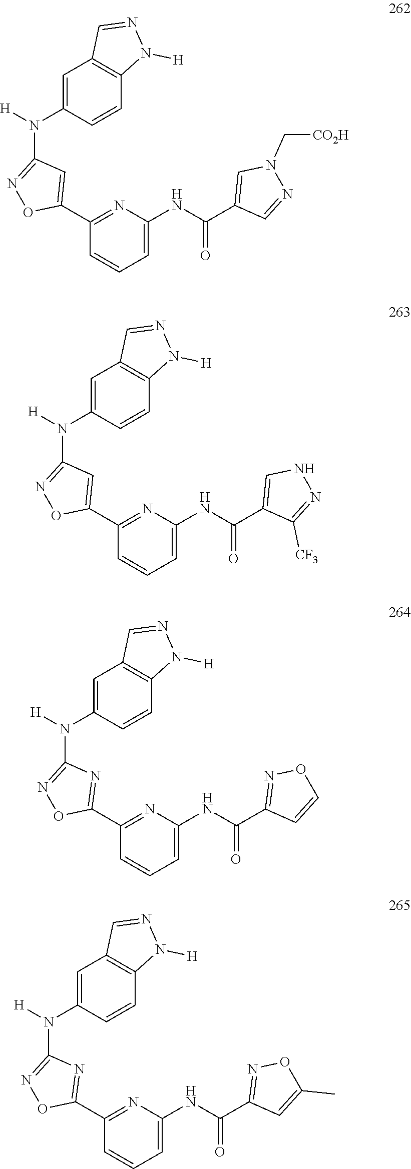

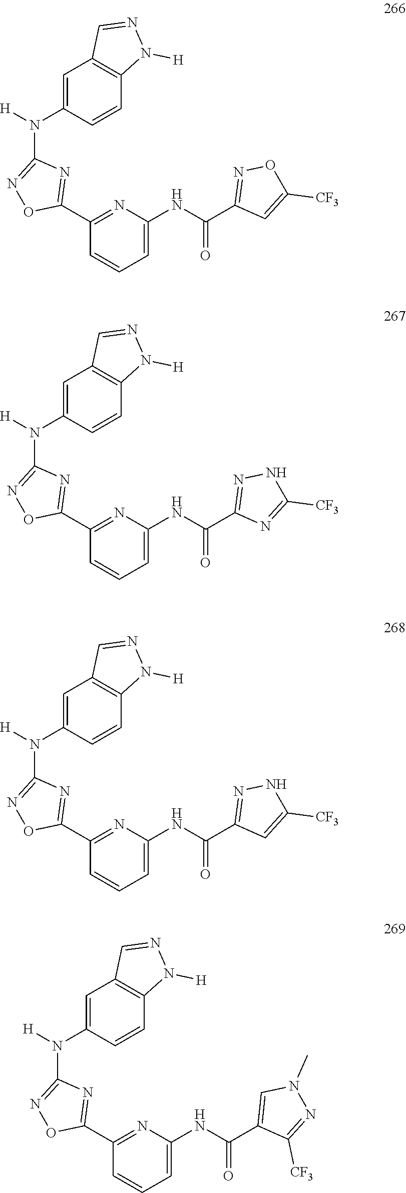 [SMILES: O=C(Nc1cccc(-c2nc(Nc3ccc4[nH]ncc4c3)no2)n1)c1cc(C(F)(F)F)on1 ; O=C(Nc1cccc(-c2nc(Nc3ccc4[nH]ncc4c3)no2)n1)c1n[nH]c(C(F)(F)F)n1 ; O=C(Nc1cccc(-c2nc(Nc3ccc4[nH]ncc4c3)no2)n1)c1cc(C(F)(F)F)[nH]n1 ; Cn1cc(C(=O)Nc2cccc(-c3nc(Nc4ccc5[nH]ncc5c4)no3)n2)c(C(F)(F)F)n1]

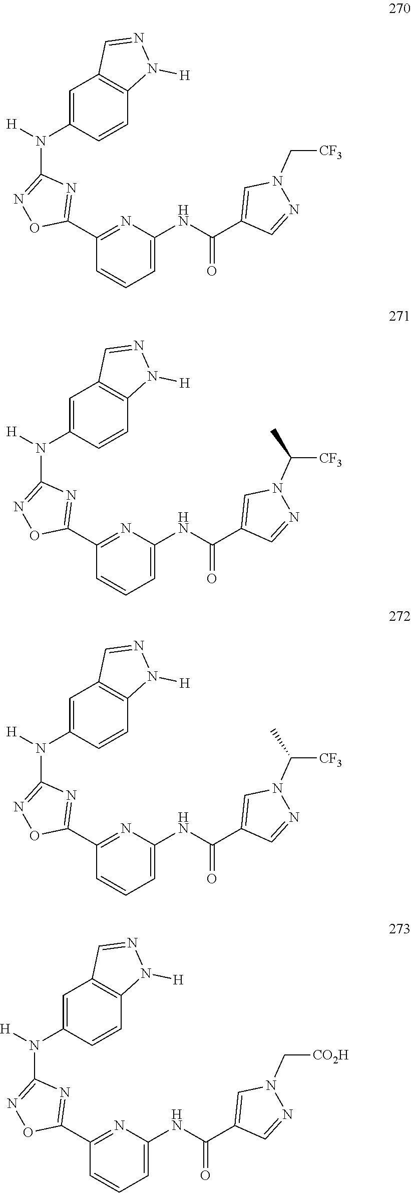 [SMILES: O=C(Nc1cccc(-c2nc(Nc3ccc4[nH]ncc4c3)no2)n1)c1cnn(CC(F)(F)F)c1 ; C[C@H](n1cc(C(=O)Nc2cccc(-c3nc(Nc4ccc5[nH]ncc5c4)no3)n2)cn1)C(F)(F)F ; C[C@@H](n1cc(C(=O)Nc2cccc(-c3nc(Nc4ccc5[nH]ncc5c4)no3)n2)cn1)C(F)(F)F ; O=C(O)Cn1cc(C(=O)Nc2cccc(-c3nc(Nc4ccc5[nH]ncc5c4)no3)n2)cn1]

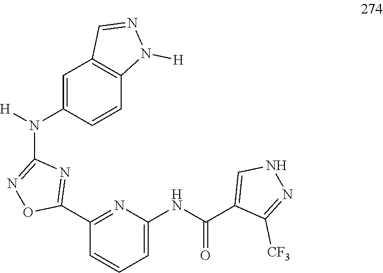 [SMILES: O=C(Nc1cccc(-c2nc(Nc3ccc4[nH]ncc4c3)no2)n1)c1c[nH]nc1C(F)(F)F]

In certain embodiments, a provided compound (a compound described herein, a compound of the present disclosure) is a compound of Formula (I), or a pharmaceutically acceptable salt, co-crystal, tautomer, stereoisomer, solvate, hydrate, polymorph, isotopically enriched compound, or prodrug thereof. In certain embodiments, a provided compound is a compound of Formula (I), or a pharmaceutically acceptable salt, tautomer, or isotopically enriched compound thereof. In certain embodiments, a provided compound is a compound of Formula (I), or a pharmaceutically acceptable salt or tautomer thereof. In certain embodiments, a provided compound is a compound of Formula (I), or a pharmaceutically acceptable salt thereof.

In certain embodiments, the compound of Formula (II) is one of the following compounds, or a pharmaceutically acceptable salt, co-crystal, tautomer, stereoisomer, solvate, hydrate, polymorph, isotopically enriched compound, or prodrug thereof:

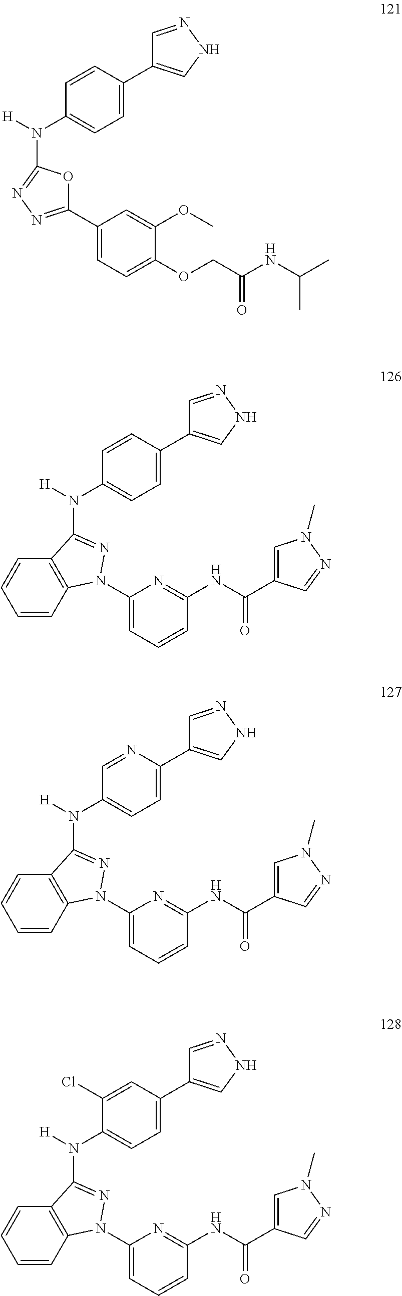 [SMILES: COc1cc(-c2nnc(Nc3ccc(-c4cn[nH]c4)cc3)o2)ccc1OCC(=O)NC(C)C ; Cn1cc(C(=O)Nc2cccc(-n3nc(Nc4ccc(-c5cn[nH]c5)cc4)c4ccccc43)n2)cn1 ; Cn1cc(C(=O)Nc2cccc(-n3nc(Nc4ccc(-c5cn[nH]c5)nc4)c4ccccc43)n2)cn1 ; Cn1cc(C(=O)Nc2cccc(-n3nc(Nc4ccc(-c5cn[nH]c5)cc4Cl)c4ccccc43)n2)cn1]

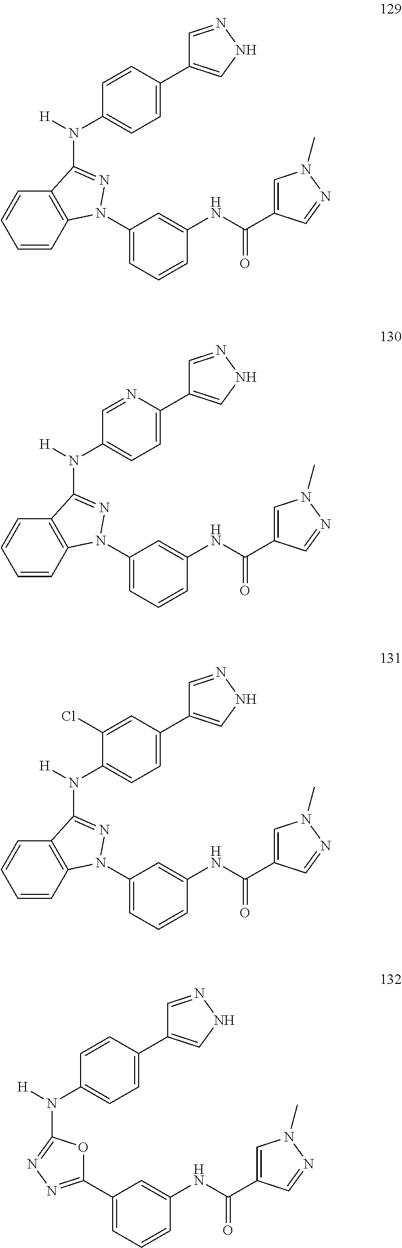 [SMILES: Cn1cc(C(=O)Nc2cccc(-n3nc(Nc4ccc(-c5cn[nH]c5)cc4)c4ccccc43)c2)cn1 ; Cn1cc(C(=O)Nc2cccc(-n3nc(Nc4ccc(-c5cn[nH]c5)nc4)c4ccccc43)c2)cn1 ; Cn1cc(C(=O)Nc2cccc(-n3nc(Nc4ccc(-c5cn[nH]c5)cc4Cl)c4ccccc43)c2)cn1 ; Cn1cc(C(=O)Nc2cccc(-c3nnc(Nc4ccc(-c5cn[nH]c5)cc4)o3)c2)cn1]

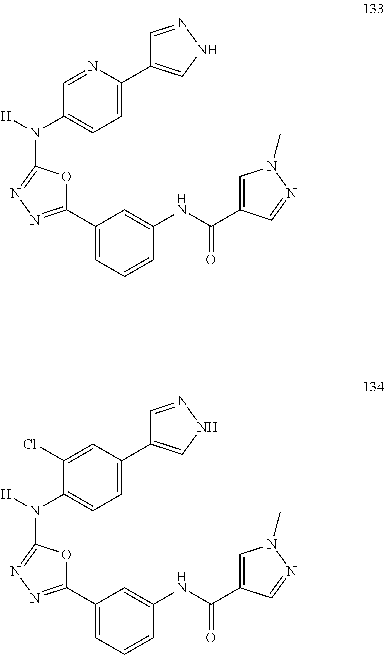 [SMILES: Cn1cc(C(=O)Nc2cccc(-c3nnc(Nc4ccc(-c5cn[nH]c5)nc4)o3)c2)cn1 ; Cn1cc(C(=O)Nc2cccc(-c3nnc(Nc4ccc(-c5cn[nH]c5)cc4Cl)o3)c2)cn1]

In certain embodiments, a provided compound (a compound described herein, a compound of the present disclosure) is a compound of Formula (II), or a pharmaceutically acceptable salt, co-crystal, tautomer, stereoisomer, solvate, hydrate, polymorph, isotopically enriched compound, or prodrug thereof. In certain embodiments, a provided compound is a compound of Formula (II), or a pharmaceutically acceptable salt, tautomer, or isotopically enriched compound thereof. In certain embodiments, a provided compound is a compound of Formula (II), or a pharmaceutically acceptable salt or tautomer thereof. In certain embodiments, a provided compound is a compound of Formula (II), or a pharmaceutically acceptable salt thereof.

In certain embodiments, the provided compounds (e.g., compounds of Formula (I) and (II)) inhibit ROCK1 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the provided compounds (e.g., compounds of Formula (I) and (II)) inhibit ROCK2 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the provided compounds (e.g., compounds of Formula (I) and (II)) selectively inhibit ROCK2 over ROCK1. In certain embodiments, the compounds are 2-fold, 5-fold, 10-fold, 20-fold, 30-fold, 40-fold, 50-fold, 60-fold, 70-fold, 80-fold, 90-fold, 100-fold, 1,000-fold, or 10,000-fold more selective inhibitors of ROCK2 over ROCK1.

### Pharmaceutical Compositions, Kits, and Administration

The present disclosure provides pharmaceutical compositions comprising a provided compound, and optionally a pharmaceutically acceptable excipient. In certain embodiments, the pharmaceutical composition described herein comprises a provided compound, and a pharmaceutically acceptable excipient.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for treating a disease or disorder associated with ROCK2 in a subject in need thereof. In certain embodiments, the effective amount is an amount effective for preventing a disease or disorder associated with ROCK2. In certain embodiments, the effective amount is an amount effective for reducing the risk of developing a disease or disorder associated with ROCK2 in a subject in need thereof. In certain embodiments, the effective amount is an amount effective for inhibiting ROCK2. In certain embodiments, inhibiting ROCK2 is inhibiting the activity (e.g., aberrant activity, such as increased activity) of ROCK2 (e.g., in a subject, tissue, biological sample, or cell).

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal.

In certain embodiments, the effective amount is an amount effective for inhibiting the activity of ROCK2 by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting the activity of ROCK2 by a range between a percentage described in this paragraph and another percentage described in this paragraph, inclusive.

In certain embodiments, the pharmaceutical composition is for use in treating a disease or disorder associated with ROCK2. In certain embodiments, the pharmaceutical composition is for use in preventing a disease or disorder associated with ROCK2. In certain embodiments, the pharmaceutical composition is for use in inhibiting ROCK2.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease or disorder associated with ROCK2 in a subject in need thereof, in preventing a disease or disorder associated with ROCK2 in a subject in need thereof, and/or in reducing the risk of developing a disease or disorder associated with ROCK2 in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compounds and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with ROCK2. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, and immunosuppressants. In certain embodiments, the additional pharmaceutical agent is an anti-inflammatory agent. In certain embodiments, the additional pharmaceutical agent is an immunotherapy. In certain embodiments, the additional pharmaceutical agent is an anti-proliferative agent. In certain embodiments, the additional pharmaceutical agent is an anti-cancer agent. In certain embodiments, the anti-cancer agents include, but are not limited to, epigenetic or transcriptional modulators (e.g., DNA methyltransferase inhibitors, HDAC inhibitors, lysine methyltransferase inhibitors), antimitotic drugs (e.g., taxanes and vinca alkaloids), cell signaling pathway inhibitors (e.g., tyrosine protein kinase inhibitors), modulators of protein stability (e.g., proteasome inhibitors), Hsp90 inhibitors, glucocorticoids, all-trans retinoic acids, anti-estrogens (e.g., tamoxifen, raloxifene, and megestrol), LHRH agonists (e.g., goscrclin and leuprolide), anti-androgens (e.g. flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DIFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²′ ATPase inhibitors (e.g., thapsigargin), thalidomide, lenalidomide, pomalidomide, tyrosine kinase inhibitors (e.g., axitinib, bosutinib, cediranib (RECENTIN™), dasatinib (SPRYCEL*), erlotinib (TARCEVA®), gefitinib (IRESSA®), imatinib (Gleevec®), lapatinib (TYKERB®, TYVERB®), lestaurtinib, neratinib, nilotinib (TASIGNA®), semaxanib, sunitinib (SUTENT®), toceranib (PALLADIA®), vandetanib (ZACTIMA®, ZD6474), vatalanib (PTK787), nilotinib (TASIGNA®), sorafenib (NEXAVAR®), everolimus (AFINITOR®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In certain embodiments, the additional pharmaceutical agent is cisplatin. In certain embodiments, the additional pharmaceutical agent is paclitaxel. In certain embodiments, the additional pharmaceutical agent is vincristine.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon 7), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDIO680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, pertuzumab, obinutuzumab, cabiralizumab, margetuximab, enoblituzumab, mogamulizumab, carlumab, bevacizumab, trastuzumab (HERCEPTIN®), bevacizumab (AVASTIN®), rituximab (RITUXAN®), cetuximab (ERBITUX®), panitumumab (VECTIBIX®), alemtuzumab (CAMPATH®), or ranibizumab (Lucentis®).

In certain embodiments, the compounds or pharmaceutical compositions described herein can be administered in combination with an anti-cancer therapy including, but not limited to, surgery, radiation therapy, and transplantation (e.g., stem cell transplantation, bone marrow transplantation).

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present dislcosure can be administered to humans and other animals orally, parenterally, intracisternally, intraperitoneally, topically, bucally, or the like, depending on the disease or disorder.

In certain embodiments, a pharmaceutical composition comprising a compound of Formula (I) or (II) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) or (II) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™) polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein. In certain embodiments, the kit further comprises a first container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In certain embodiments, the kits are useful for treating a disease or disorder associated with ROCK2 in a subject in need thereof. In certain embodiments, the kits are useful for preventing a disease or disorder associated with ROCK2 in a subject in need thereof. In certain embodiments, the kits are useful for reducing the risk of developing a disease or disorder associated with ROCK2 in a subject in need thereof. In certain embodiments, the kits are useful for inhibiting the activity (e.g., aberrant activity, such as increased activity) of ROCK2 in a subject or cell.

In certain embodiments, a kit described herein further includes instructions for using the pharmaceutical composition or compound. A kit described herein may also include information as required by a regulatory agency such as the U.S. Food and Drug Administration (FDA). In certain embodiments, the information included in the kits is prescribing information. In certain embodiments, the kits and instructions provide for treating a disease or disorder associated with ROCK2 in a subject in need thereof. In certain embodiments, the kits and instructions provide for preventing a disease or disorder associated with ROCK2 in a subject in need thereof. In certain embodiments, the kits and instructions provide for reducing the risk of developing a disease or disorder associated with ROCK2 in a subject in need thereof. In certain embodiments, the kits and instructions provide for inhibiting the activity (e.g., aberrant activity, such as increased activity) of ROCK2 in a subject or cell. A kit described herein may include one or more additional pharmaceutical agents described herein as a separate composition.

### Methods of Use

The present disclosure also provides methods for treating diseases or disorders associated with ROCK2 in a subject in need thereof, the methods comprising administering to the subject an effective amount of a provided compound or pharmaceutical composition.

The present disclosure also provides methods for preventing diseases or disorders associated with ROCK2 in a subject in need thereof, the methods comprising administering to the subject an effective amount of a provided compound or pharmaceutical composition.

In certain embodiments, the disease or disorder associated with ROCK2 is a fibrotic disorder, autoimmune disease, inflammatory condition, an edema, an ophthalmic disease, cardiovascular disease, central nervous system disorder, or cancer.

In certain embodiments, the disease or disorder associated with ROCK2 is a fibrotic disorder. In certain embodiments, the disease or disorder associated with ROCK2 is pulmonary fibrosis, cystic pulmonary fibrosis, idiopathic pulmonary fibrosis, radiation induced lung injury, liver fibrosis including cirrhosis, cardiac fibrosis including arterial fibrosis, endomyocardial fibrosis, old myocardial infraction, arterial stiffness, atherosclerosis, restenosis, arthrofibrosis, Crohn's disease, myelofibrosis, Peyronie's diseases, nephrogenic systemic fibrosis, progressive massive fibrosis, retroperitoneal cavity fibrosis, schleroderma/systemic sclerosis, mediastinal fibrosis, Keloids and hypertrophic scars, glial scaring, or renal fibrosis.

In certain embodiments, the disease or disorder associated with ROCK2 is a central nervous system disorder. In certain embodiments, the disease or disorder associated with ROCK2 is Huntington's disease, Parkinson's disease, Alzheimer's disease, Amyotrophic lateral sclerosis (ALS), Batten disease, dementia, spinal muscular atrophy, motor neurone diseases, spinocerebellar ataxia, acute or chronic pain, dementia, neuronal degeneration, spinal cord injury, or cerebral vasospasm.

In certain embodiments, the disease or disorder associated with ROCK2 is an ophthalmic disease. In certain embodiments, the disease or disorder associated with ROCK2 is glaucoma.

In certain embodiments, the disease or disorder associated with ROCK2 is an autoimmune disease. In certain embodiments, the disease or disorder associated with ROCK2 is rheumatoid arthritis, multiple sclerosis, systemic lupus erythematosus, psoriasis, Crohn's disease, atopic dermatitis, eczema, or graft-versus-host disease (GVHD).

In certain embodiments, the disease or disorder associated with ROCK2 is an inflammatory condition. In certain embodiments, the disease or disorder associated with ROCK2 is asthma, cardiovascular inflammation, renal inflammation, or arteriosclerosis.

In certain embodiments, the disease or disorder associated with ROCK2 is a cardiovascular disease. In certain embodiments, the disease or disorder associated with ROCK2 is hypertension, atherosclerosis, angina, arterial obstruction, peripheral arterial disease, peripheral circulatory disorder, cerebral cavernous malformation, restenosis, cardiac hypertrophy, ocular hypertension, cerebral ischemia, cerebral vasospasm, acute respiratory distress syndrome (ARDS), or erectile dysfunction.

In certain embodiments, the disease or disorder associated with ROCK2 is an edema. In certain embodiments, the disease or disorder associated with ROCK2 is lymphedema. In certain embodiments, the lymphedema is caused at least by a parasitic disease. In certain embodiments, the lymphedema is caused at least by filariasis. In certain embodiments, the lymphedema is caused at least by elephantiasis. In certain embodiments, the disease or disorder associated with ROCK2 is angioedema, brain edema, CHAPLE syndrome, cardiac edema, hydrops fetalis, inflammatory edema, macular edema, myxedema, pulmonary edema, peripheral edema, periorbital edema, or cutaneous edema. In certain embodiments, the disease or disorder associated with ROCK2 is hereditary angioedema, cystoid macular edema, Irvine-Gass syndrome, diabetic macular edema, or pedal edema. In certain embodiments, the edema is caused at least by prolonged sitting or staying in one position, excessive intake of sodium, menstruation, or pregnancy, or a combination thereof. In certain embodiments, the edema is a side effect of a high blood pressure medication, nonsteroidal anti-inflammatory drug, steroid, estrogen, or thiazolidinedione. In certain embodiments, the edema is caused at least by congestive heart failure, cirrhosis, kidney disease, kidney damage, weakness or damage to veins in the legs, inadequate lymphatic system, or severe and/or long-term protein deficiency, or a combination thereof. In certain embodiments, the method is a method of reducing a symptom of edema (e.g., swelling of the tissues under (e.g., directly under) the skin, stretched skin, shiny skin, skin that retains a dimple after being pressed for several seconds, or increased abdominal size). In certain embodiments, the skin is the skin of the legs or arms.

In certain embodiments, the disease or disorder associated with ROCK2 is cancer. In certain embodiments, the disease or disorder associated with ROCK2 is a solid tumor. In certain embodiments, the disease or disorder associated with ROCK2 is a hematological malignancy.

The present disclosure also provides methods of inhibiting the activity of ROCK2 comprising contacting ROCK2 with an effective amount of a provided compound or pharmaceutical composition. In certain embodiments, the ROCK2 is in vitro. In certain embodiments, the ROCK2 is in vivo. In certain embodiments, the ROCK2 is in a cell (e.g., a human cell). In certain embodiments, the cell is in vitro. In certain embodiments, the cell is in vivo.

In another aspect, the present disclosure provides methods of screening a library of compounds comprising performing an assay on a provided compound and an additional compound, wherein the additional compound is different from the provided compound. In certain embodiments, the assay is an in vitro assay. In certain embodiments, the assay is a biochemical assay. In certain embodiments, the assay is an enzymatic assay. In certain embodiments, the assay is a cell-based assay. In certain embodiments, the assay is an assay described herein. In certain embodiments, the methods of screening a library of compounds further comprise identifying an additional compound that is useful in a method described herein.

The present disclosure also provides uses of a provided compound in a method described herein. The present disclosure also provides uses of a provided pharmaceutical composition in a method described herein. The present disclosure also provides a provided compound for use in a method described herein. The present disclosure also provides uses of a provided pharmaceutical composition in a method described herein. The present disclosure also provides a provided pharmaceutical composition for use in a method described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthetic Methods

### General Details.

As used herein the following terms have the meanings given: “DMF” refers to N,N-dimethylformamide; “EtOAc” refers to ethyl acetate; “DCM” refers to dichloromethane; “DMSO” refers to dimethylsulfoxide; “THF” refers to tetrahydrofuran; “2-MeTHF” refers to 2-methyltetrahydrofuran; “MeOH” refers to methanol; “EtOH” refers to ethanol; “MeCN” refers to acetonitrile; “DIPEA” or “DIEA” refers to N,N-diisopropylethylamine; “TEA” refers to trimethylamine; “Py” refers to pyridine; “t-BuOK” refers to potassium tert-butoxide; “KOAc” refers to potassium acetate; “n-BuLi” refers to n-butyllithium; “TFA” refers to trifluoroacetic acid, “FA” refers to formic acid; “Ac₂O” refers to acetic anhydride; “DHP” refers to 3,4-dihydro-2H-pyran; “NCS” refers to 1-chloropyrrolidine-2,5-dione; “Mel” refers to methyl iodide; “Fe” refers to iron powder; “TosCl” refers to paratoluensulfonyl chloride; “HATU” refers to 1-[bis(dimethylamino)methylene]-1H-1,2,3-triazolo[4,5-b]pyridinium-3-oxide hexafluorophosphate; “EDCI” refers to 1-ethyl-3-(3-dimethylamino-propyl)-carbodiimide hydrochloride; “PyBOP” refers to Benzotriazole-1-yl-oxy-tris-pyrrolidino-phosphonium hexafluoroph; “Xantphos” refers to 4,5-bis(diphenylphosphino)-9,9-dimethylxanthene; “Pd2dba3” refers to Tris(dibenzylideneacetone)dipalladium; “Pd(PPh₃)₄” refers to Tetrakis(triphenylphosphine)palladium; “Pd(dppf)Cl2” refers to [1,1′-Bis(diphenylphosphino)ferrocene]dichloropalladium(II); “HPLC” refers to high performance liquid chromatography; “LCMS” or “LC-MS” refers to liquid chromatography/mass spectrometry; “min” refers to minute; “Pet. Ether” refers to petroleum ether; “TLC” refers to thin layer chromatography; “Rf refers to retention factor; “RT” refers to retention time; “r.t.” refers to room temperature.

Solvents, reagents and starting materials were purchased from commercial vendors and used as received unless otherwise described. All reactions were performed at r.t. unless otherwise stated.

Compound identity and purity confirmations were performed by LCMS UV using a SHIMADZU LCMS-2020. The PDA wavelength was 220 & 254 nM and the MS was in positive electrospray mode (m/z: 100-1000). The aliquot was injected onto a HPLC column (Kinetex® EVO C18 2.1*30 mm, 2.6 um) in sequence maintained at 50° C. The samples were eluted at a flow rate of 1.5 mL/min with a mobile phase system composed of A (0.0375% (v/v) TFA in water) and B (0.01875% (v/v) TFA in Acetonitrile) according to the gradients outlined in Table 1 below. Retention times RT are reported in min.

TABLE 1
Exemplary HPLC parameters

| | | |
|---|---|---|
| Time (min) | B (%) | Flow (mL/min) |

Method A

| | | |
|---|---|---|
| 0.0 | 5 | 1.5 |
| 0.8 | 95 | 1.5 |
| 1.09 | 95 | 1.5 |
| 1.10 | 5 | 1.5 |
| 1.20 | 5 | 1.5 |

Method B

| | | |
|---|---|---|
| 0.0 | 0 | 1.5 |
| 0.8 | 60 | 1.5 |
| 1.09 | 60 | 1.5 |
| 1.10 | 0 | 1.5 |
| 1.20 | 0 | 1.5 |

Compound identity and purity confirmations were performed by LCMS UV using a SHIMADZU LCMS-2020. The PDA wavelength was 220 & 254 nM and the MS was in positive electrospray mode (m/z: 100-1000). The aliquot was injected onto a HPLC column (XBridge C18 2.1*50 mm, 5 um) in sequence maintained at 40° C. The samples were eluted at a flow rate of 1.5-2.0 mL/min with a mobile phase system composed of A (0.025% (v/v) NH₃·H₂O in water) and B (Acetonitrile) according to the gradients outlined in Table 2 below. Retention times RT are reported in min.

TABLE 2
Exemplary HPLC parameters
Method C

| Time (min) | B (%) | Flow (mL/min) |
|---|---|---|
| 0.00 | 5 | 1.5 |
| 0.80 | 95 | 1.5 |
| 1.10 | 95 | 2.0 |
| 1.11 | 5 | 2.0 |
| 1.20 | 5 | 2.0 |

Compound identity and purity confirmations were performed by LCMS UV using an Agilent 1260G6125B. The DAD wavelength was 220 & 254 nM and the MS was in positive electrospray mode (m/z: 100-1000). The aliquot was injected onto a HPLC column (XBridge C18 2.1*50 mm, 5 um) in sequence maintained at 40° C. The samples were eluted at a flow rate of 1.5-2.0 mL/min with a mobile phase system composed of A (0.025% (v/v) NH₃·H₂O in water) and B (Acetonitrile) according to the gradients outlined in Table 3 below. Retention times RT are reported in min.

TABLE 3
Exemplary HPLC parameters
Method D

| Time (min) | B (%) | Flow (mL/min) |
|---|---|---|
| 0.0 | 5 | 1.5 |
| 0.70 | 95 | 1.8 |
| 1.00 | 95 | 2 |
| 1.01 | 5 | 2 |
| 1.20 | 5 | 2 |

NMR was also used to characterize final compounds. ¹H NMR spectra were obtained at r.t., unless otherwise stated, on a Bruker AVANCE III 400 with a 5 mm BBO probe with Z gradients, a Bruker AVANCE III HD 400 with a 5 mm BBO probe with Z gradients, a Bruker AVANCE NEO 400 with either a 5 mm BBO probe or 5 mm BBO prodigy cryoprobe with Z gradients, a Bruker NEO NANOBAY 400 with either a 5 mm BBO probe or 5 mm BBO iProbe with Z gradients. Chemical shifts are reported in ppm and referenced to either DMSO-d₆ (2.50 ppm), CDCl₃ (7.26 ppm) or MeOD-d₄ (3.31 ppm). NH or OH signals that exchange with deuterated solvent are not reported.

Optionally, compound Rf values on silica thin layer chromatography (TLC) plates were measured. Compound purification was performed by flash column chromatography on silica or by preparative HPLC. HPLC purification was performed using either Gilson-281 or Shimadzu LC-20AP in positive electrospray mode (m/z: 100-1000) with a Shimadzu SPD-20A. Samples were eluted at a flow rate of 25 mL/min on a Phenomenex Luna C18 150*25 mm*10 um column with a mobile phase system composed of: 1. Basic conditions: A (0.05% ammonia (v/v) in H₂O) and B (Acetonitrile), 2. TFA conditions: A (0.075% TFA (v/v) in H₂O and B (Acetonitrile), 3. A (0.225% FA (v/v) in H₂O and B (Acetonitrile), 4. HCl conditions: A (0.05% HCl (v/v) in H₂O and B (Acetonitrile), 5. Neutral conditions: A (H₂O) and B (Acetonitrile) or A (10 mmol NH₄HCO₃) in H₂O and B (Acetonitrile) according to the different linear gradient for samples.

General route for the synthesis of Intermediates 1-4:

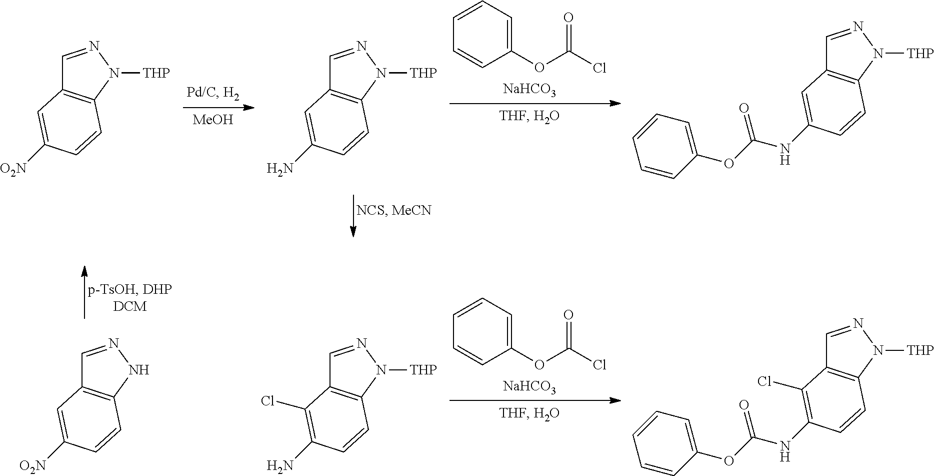

### Step 1: 5-nitro-1-(tetrahydro-2H-pyran-2-yl)-1H-indazole

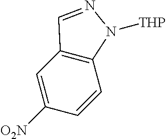

To a suspension of 5-nitro-1H-indazole (35 g, 215 mmol) in DCM (450 mL) was added DHP (54 g, 644 mmol) at r.t., and then p-TsOH (3.69 g, 21.5 mmol) was added by potion. The reaction was stirred at 30° C. for 16 hr. O₂N The reaction was poured into brine (300 mL). The organic layer was washed with brine (2×300 mL), dried over Na₂SO₄, filtered and concentrated. The residue was triturated with Pet. Ether (500 mL) and stirred for 0.5 h. The mixture was filtered and solid was collected and dried over under reduced pressure to give 5-nitro-1-(tetrahydro-2H-pyran-2-yl)-1H-indazole (42.2 g, 171 mmol, 80% yield) as a brown solid.

### Intermediate 1: 1-(tetrahydro-2H-pyran-2-yl)-1H-indazol-5-amine

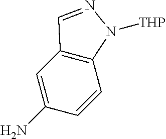

To a solution of 5-nitro-1-(tetrahydro-2H-pyran-2-yl)-1H-indazole (5 g, 20.22 mmol) in MeOH (100 mL) was added palladium, 10 wt. % on carbon powder (0.5 g) under N₂. The suspension was degassed under reduced H₂N pressure and purged with H₂ several times. The mixture was stirred under H₂ (30 psi) at r.t. for 16 hr. The reaction mixture was filtered through a pad of Celite, and the mother liquid was concentrated to give a residue which was triturated with Pet. Ether/EtOAc/MeOH (50 mL/10 mL/5 mL) for 1 hr. The mixture was filtered to give 1-(tetrahydro-2H-pyran-2-yl)-1H-indazol-5-amine (4 g, 17.49 mmol, 87% yield) as a brown solid. LC-MS (ES⁺, Method A), 0.27 min, m/z 218.3 [M+H]⁺.

### Intermediate 2: phenyl (1-(tetrahydro-2H-pyran-2-yl)-1H-indazol-5-yl)carbamate

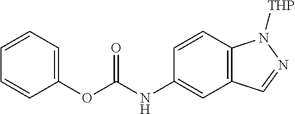

To a solution of 1-(tetrahydro-2H-pyran-2-yl)-1H-indazol-5-amine (2 g, 9.21 mmol), NaHCO₃ (1.55 g, 18.41 mmol) in THF (10 mL) and H₂O (10 mL) was added phenyl carbonochloridate (1.59 g, 10.13 mmol) at 0° C., and the reaction was stirred at 0° C. for 0.5 hr. The reaction mixture was filtered and the filter cake was dried to give phenyl (1-(tetrahydro-2H-pyran-2-yl)-1H-indazol-5-yl)carbamate (2.50 g, 7.41 mmol, 80% yield) as a pink solid. LC-MS (ES+, Method D), 0.88 min, m/z 338.0 [M+H]⁺.

### Intermediate 3: 4-chloro-1-(tetrahydro-2H-pyran-2-yl)-1H-indazol-5-amine

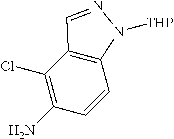

To a solution of 1-(tetrahydro-2H-pyran-2-yl)-1H-indazol-5-amine (5 g, 23.01 mmol,) in MeCN (80 mL) was added NCS (6.15 g, 46.03 mmol) at 0° C., the reaction solution was stirred at 0° C. for 1 hr. The reaction mixture was quenched by addition sodium sulfite aqueous (10% wt, 80 mL) and then extracted with EtOAc (3×50 mL). The combined organic layers were washed with brine (40 mL), dried over anhydrous sodium sulfate, filtered the solvent removed under reduced pressure to give a residue. The residue was purified by preparative HPLC (30-80% MeCN in H₂O) to give 4-chloro-1-(tetrahydro-2H-pyran-2-yl)-1H-indazol-5-amine (1.7 g, 6.75 mmol, 29.4% yield). LC-MS (ES+, Method A), 0.51 min, m/z 252.1 [M+H]⁺.

### Intermediate 4: phenyl (4-chloro-1-(tetrahydro-2H-pyran-2-yl)-1H-indazol-5-yl)carbamate

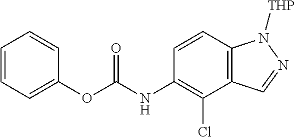

To a solution of 4-chloro-1-(tetrahydro-2H-pyran-2-yl)-1H-indazol-5-amine (1 g, 3.97 mmol) and NaHCO₃ (667 mg, 7.95 mmol) in THE (10 mL) and H₂O (10 mL) was added phenyl carbonochloridate (684 mg, 4.37 mmol) at 0° C. The reaction solution was stirred at 0° C. for 0.5 hr. The reaction solution was filtered and the filter cake was washed by EtOAc (20×3 mL) to give phenyl (4-chloro-1-(tetrahydro-2H-pyran-2-yl)-1H-indazol-5-yl)carbamate (1 g, 2.69 mmol, 67.7% yield) as a brown solid.

### Intermediates 5: 1H-imidazole-4-carbonyl chloride

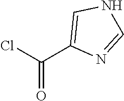

To a mixture of 1H-imidazole-4-carboxylic acid (100 mg, 892 μmol) in DCM (10 mL) was added (COCl)₂ (1.45 g, 11.4 mmol) and DMF (97.82 mg, 1.34 mmol) at r.t. under N₂. The mixture was stirred at r.t. for 30 mins. The reaction mixture was concentrated to give 1H-imidazole-4-carbonyl chloride (115 mg, 881 μmol) as a yellow solid.

General route for the synthesis of Intermediates 6:

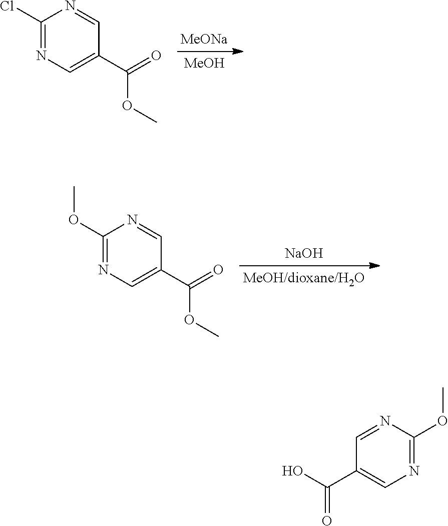

### Step 1: methyl 2-methoxypyrimidine-5-carboxylate

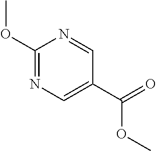

To a solution of methyl 2-chloropyrimidine-5-carboxylate (1 g, 5.79 mmol) in MeOH (10 mL) was added sodium methoxide in CH₃OH (5.4 M, 1.07 mL) at r.t. The reaction was stirred at r.t. for 1 h. The reaction solvent removed under reduced pressure and the residue was washed by H₂O (20 mL). The mixture was filtered and the filter cake give methyl 2-methoxypyrimidine-5-carboxylate (950 mg, 5.65 mmol, 98% yield) as a light yellow solid. LC-MS (ES⁺, Method A), 0.31 min, m/z 169.1 [M+H]⁺.

### Intermediates 6: 2-methoxypyrimidine-5-carboxylic acid

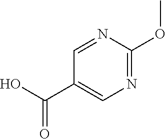

To a solution of methyl 2-methoxypyrimidine-5-carboxylate (500 mg, 2.97 mmol) in MeOH (6 mL), dioxane (4 mL) and H₂O (2 mL) was added NaOH (238 mg, 5.95 mmol) at r.t. The reaction was stirred at r.t. for 1 hr. The reaction mixture was acidified to pH=2 by HCl (1 M) aqueous solution and the solvent was removed under reduced pressure to give 2-methoxypyrimidine-5-carboxylic acid (500 mg, crude) as a yellow solid. LC-MS (ES⁺, Method A), 0.14 min, m/z 155.1 [M+H]⁺.

### General Route or the Synthesis of Examples 1-9:

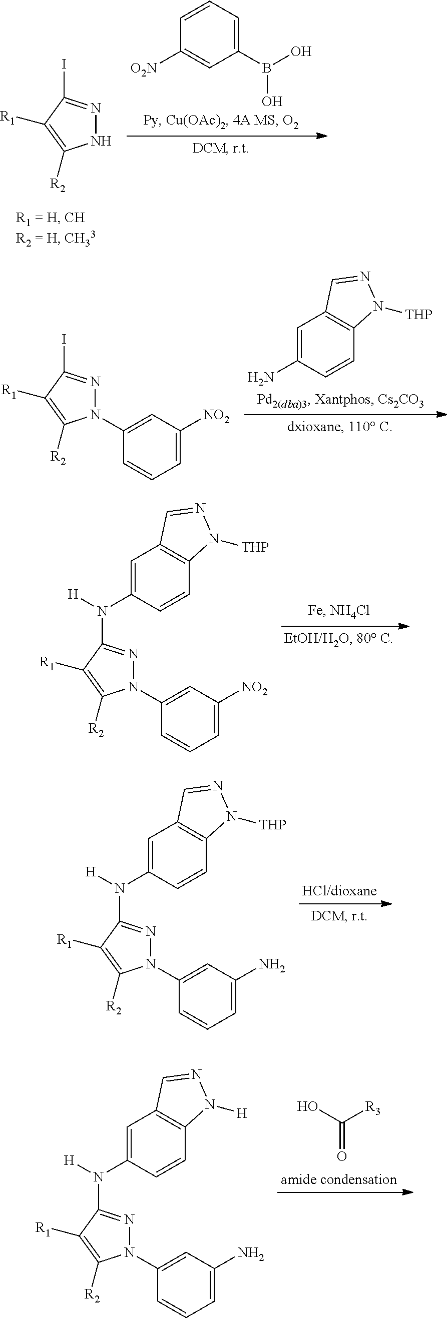

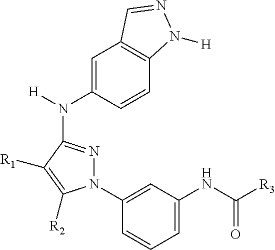

### Step 1: 3-iodo-1-(3-nitrophenyl)-1H-pyrazole

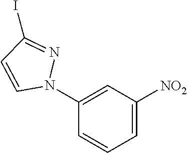 [SMILES: O=[N+]([O-])c1cccc(-n2ccc(I)n2)c1]

To a mixture of (3-nitrophenyl)boronic acid (5.16 g, 30.93 mmol) and 3-iodo-1H-pyrazole (3 g, 15.47 mmol) in DCM (10 mL) was added 4A NO₂ molecular sieve (10 g, 1.55 mmol), Py (2.45 g, 30.93 mmol) and Cu(OAc)₂ (4.21 g, 23.20 mmol) at r.t. The mixture was stirred at r.t. for 16 hr under O₂ (15 psi). The reaction mixture was poured into EtOAc (500 mL) and filtered to remove 4 Å MS and copper salt and the mother liquid was concentrated to give a residue. The residue was purified by flash silica gel chromatography eluting with 0-10% EtOAc in Pet. Ether to give 3-iodo-1-(3-nitrophenyl)-1H-pyrazole (6.3 g, crude). The crude was re-purified by reversed phase MPLC (FA conditions) to give 3-iodo-1-(3-nitrophenyl)-1H-pyrazole (4.6 g, 14.60 mmol, 94% yield) as a yellow solid. LC-MS (ES⁺, Method A), 0.96 min, m/z 316.0 [M+H]⁺.

### Step 2: N-(1-(3-nitrophenyl)-1H-pyrazol-3-yl)-1-(tetrahydro-2H-pyran-2-yl)-1H-indazol-5-amine

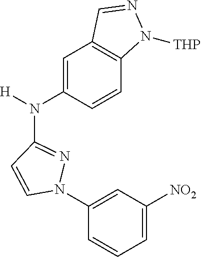

To a mixture of 3-iodo-1-(3-nitrophenyl)pyrazole (2.5 g, 7.93 mmol) and 1-tetrahydropyran-2-ylindazol-5-amine (1.74 g, 7.93 mmol) in dioxane (50 mL) was added Xantphos (459 mg, 793.48 μmol), Cs₂CO₃ (5.17 g, 15.87 mmol) and Pd₂(dba)₃ (727 mg, 793.48 μmol) at r.t. under N₂. The suspension was degassed under reduced pressure and purged with N₂ for 5 min. The mixture was heated to 110° C. and stirred for 16 hr. The mixture was poured into EtOAc (150 mL) and water (300 mL). The organic layer was separated and the water phase was extracted with EtOAc (3×150 mL). The combined organic phase was washed with brine (200 mL), dried with anhydrous sodium sulfate, filtered and concentrated under reduced pressure. The residue was purified by flash silica gel chromatography eluting with 0-30% EtOAc in Pet. Ether to give N-(1-(3-nitrophenyl)-1H-pyrazol-3-yl)-1-(tetrahydro-2H-pyran-2-yl)-1H-indazol-5-amine (1.9 g, 4.28 mmol, 54% yield) as a yellow solid. LC-MS (ES+, Method A), 1.0 min, m/z 405.2 [M+H]⁺.

### Step 3: N-(1-(3-aminophenyl)-1H-pyrazol-3-yl)-1-(tetrahydro-2H-pyran-2-yl)-1H-indazol-5-amine

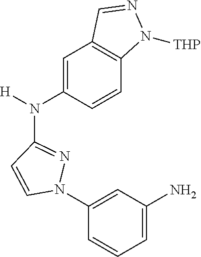

To a mixture of N-[1-(3-nitrophenyl)pyrazol-3-yl]-1-tetrahydropyran-2-yl-indazol-5-amine (1.3 g, 3.09 mmol) in EtOH (80 mL) and H₂O (16 mL) was added NH₄Cl (990 mg, 18.52 mmol) at r.t. under N₂. The mixture was heated to 50° C. and Fe (948 mg, 16.97 mmol) was added. The mixture was stirred at 80° C. for 1 hr. The reaction mixture was cooled to r.t. and EtOAc (100 mL) was added into the mixture, then, filtered through a pad of Celite. The filtered cake was washed with EtOAc (100 mL). The mother liquid was concentrated to give a residue. The residue was poured into water (100 mL) and extracted with EtOAc (3×80 mL). The combined organic layer was washed with brine (100 mL), dried with anhydrous sodium sulfate, filtered and concentrated under reduced pressure to give N-(1-(3-aminophenyl)-1H-pyrazol-3-yl)-1-(tetrahydro-2H-pyran-2-yl)-1H-indazol-5-amine (1.25 g, 2.87 mmol, 93% yield) as red oil. LC-MS (ES⁺, Method A), 0.86 min, m/z 375.2 [M+H]⁺.

### Step 4: N-(1-(3-aminophenyl)-1H-pyrazol-3-yl)-1H-indazol-5-amine

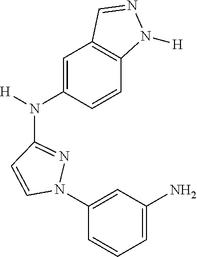 [SMILES: Nc1cccc(-n2ccc(Nc3ccc4[nH]ncc4c3)n2)c1]

To a mixture of N-[1-(3-aminophenyl)pyrazol-3-yl]-1-II N-H tetrahydropyran-2-yl-indazol-5-amine (1.25 g, 2.87 mmol) in DCM (25 mL) and MeOH (25 mL) was added HCl/dioxane (4 M, 25 mL) at 25° C. under N₂. The mixture was stirred at r.t. for 2 hr. The reaction mixture was concentrated to give a residue. EtOAc (50 mL) was added into the residue, and stirred at r.t. for 1 hr. The mixture was filtered and the solid was collected to give N-(1-(3-aminophenyl)-1H-pyrazol-3-yl)-1H-indazol-5-amine (510 mg, 1.49 mmol, 52% yield) as a yellow solid. LC-MS (ES⁺, Method A), 0.70 min, m/z 291.2 [M+H]⁺.

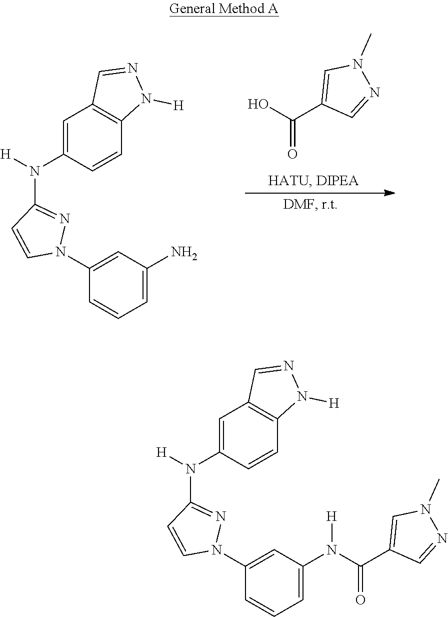

### Example 1: N-(3-(3-((1H-indazol-5-yl)amino)-1H-pyrazol-1-yl)phenyl)-1-methyl-1H-pyrazole-4-carboxamide

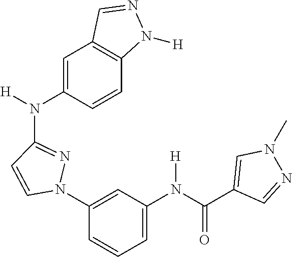 [SMILES: Cn1cc(C(=O)Nc2cccc(-n3ccc(Nc4ccc5[nH]ncc5c4)n3)c2)cn1]

To a mixture of N-(1-(3-aminophenyl)-1H-pyrazol-3-yl)-1H-indazol-5-amine (100 mg, 293 μmol) and 1-methylpyrazole-4-carboxylic acid (36.9 mg, 293 μmol) in DMF (3 mL) was added DIPEA (114 mg, 878 μmol) and HATU (167 mg, 439 μmol) at r.t. under N₂. The mixture was stirred at r.t. for 16 hr. The reaction mixture was poured into water (30 mL), and extracted with EtOAc (3×20 mL). The combined organic phase was washed with brine (30 mL), dried with anhydrous sodium sulfate, filtered and concentrated under reduced pressure. The residue was dissolved in MeOH (5 mL) and H₂O (0.5 mL) and K₂CO₃ (100 mg) was added, and the mixture was stirred at r.t. for 0.5 hr, then concentrated to give a residue. The residue was purified by purified by preparative HPLC (25-55% MeCN in H₂O) to give N-(3-(3-((1H-indazol-5-yl)amino)-1H-pyrazol-1-yl)phenyl)-1-methyl-1H-pyrazole-4-carboxamide (32.6 mg, 78.6 μmol, 27% yield) as a gray solid. LC-MS (ES⁺, Method A), 0.87 min, m/z 399.4 [M+H]⁺. ¹H NMR (400 MHz, DMSO-d₆) δ 12.79 (s, 1H), 9.98 (s, 1H), 8.72 (s, 1H), 8.36 (s, 1H), 8.26 (d, J=2.4 Hz, 1H), 8.22 (t, J=2.0 Hz, 1H), 8.12 (d, J=1.6 Hz, 1H), 8.07 (s, 1H), 7.95 (s, 1H), 7.58-7.61 (m, 1H), 7.50-7.46 (m, 1H), 7.45-7.38 (m, 2H), 7.31 (dd, J=2.0, 8.8 Hz, 1H), 6.11 (d, J=2.8 Hz, 1H), 3.92 (s, 3H).

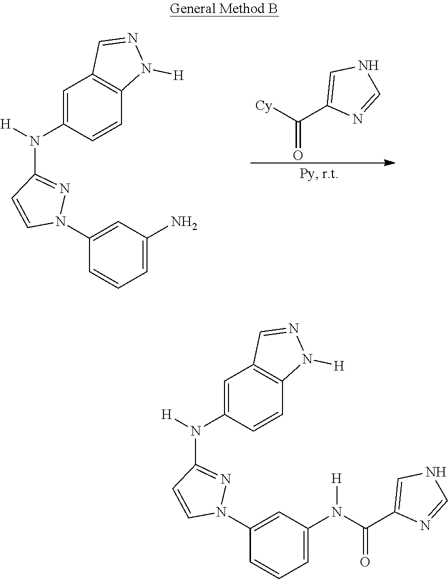

### Example 2: N-(3-(3-((1H-indazol-5-yl)amino)-1H-pyrazol-1-yl)phenyl)-1H-imidazole-4-carboxamide

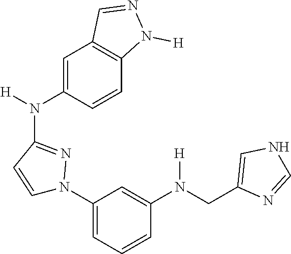 [SMILES: c1cc(NCc2c[nH]cn2)cc(-n2ccc(Nc3ccc4[nH]ncc4c3)n2)c1]

To a mixture of N-(1-(3-aminophenyl)-1H-pyrazol-3-yl)-1H-indazol-5-amine (100 mg, 293 μmol) in Py (5 mL) was added a solution of 1H-imidazole-4-carbonyl chloride (174 mg, 1.33 mmol) in Py (5 mL) at r.t. and the mixture was stirred at r.t. for 16 hr. The reaction mixture was cooled to 0° C. and poured into ice-water (50 mL) and the mixture was extracted with EtOAc (3×50 mL). The combined organic phase was washed with brine (50 mL), dried with anhydrous sodium sulfate, filtered and concentrated under reduced pressure. The residue was purified by preparative HPLC (20-40% MeCN in H₂O) to give N-(3-(3-((1H-indazol-5-yl)amino)-1H-pyrazol-1-yl)phenyl)-1H-imidazole-4-carboxamide (63.6 mg, 162.15 μmol, 55% yield) as a yellow solid. LC-MS (ES+, Method A), 0.79 min, m/z 385.4 [M+H]⁺. ¹H NMR (400 MHz, DMSO-d₆) δ 12.83-12.75 (m, 1H), 12.75-12.59 (m, 1H), 9.93 (s, 1H), 8.72 (s, 1H), 8.34 (s, 1H), 8.24 (d, J=2.4 Hz, 1H), 8.11 (d, J=1.6 Hz, 1H), 7.94 (s, 1H), 7.85 (s, 2H), 7.67-7.71 (m, 1H), 7.52-7.47 (m, 1H), 7.45-7.37 (m, 2H), 7.32 (dd, J=2.0, 9.2 Hz, 1H), 6.12 (d, J=2.4 Hz, 1H).

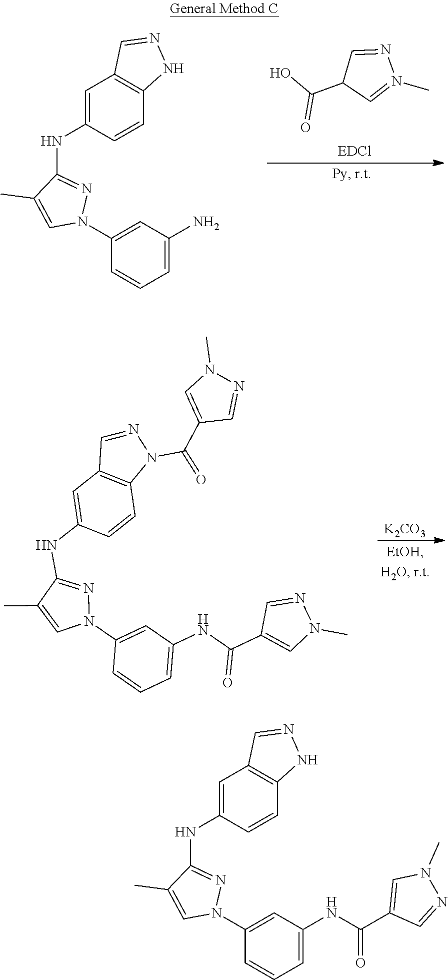

### Step 1: 1-methyl-N-(3-(4-methyl-3-((1-(1-methyl-1H-pyrazole-4-carbonyl)-1H-indazol-5-yl)amino)-1H-pyrazol-1-yl)phenyl)-1H-pyrazole-4-carboxamide

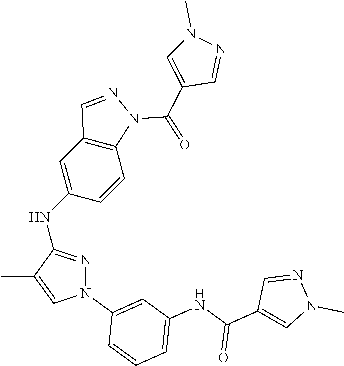

To a solution of N-(1-(3-aminophenyl)-4-methyl-1H-pyrazol-3-yl)-1H-indazol-5-amine (150 mg, 492.86 μmol) and 1-methyl-1H-pyrazole-4-carboxylic acid (93.23 mg, 739.28 μmol) in Py (9 mL) was added EDCI (236.20 mg, 1.23 mmol). The mixture was stirred at r.t. for 16 hr. The residue was partitioned between water (20 mL) and EtOAc (20 mL). The organic layer was separated and the aqueous layer was extracted with EtOAc (2×20 mL). The combined organics were washed with washed with brine (60 mL), dried over sodium sulfate, filtered and the solvent removed under reduced pressure to give 1-methyl-N-(3-(4-methyl-3-((1-(1-methyl-1H-pyrazole-4-carbonyl)-1H-indazol-5-yl)amino)-1H-pyrazol-1-yl)phenyl)-1H-pyrazole-4-carboxamide (256 mg, crude) as a yellow solid.

### Example 3: N-(3-(3-((1H-indazol-5-yl)amino)-4-methyl-1H-pyrazol-1-yl)phenyl)-1-methyl-1H-pyrazole-4-carboxamide

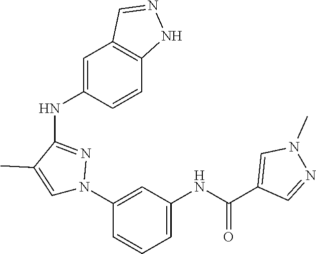 [SMILES: Cc1cn(-c2cccc(NC(=O)c3cnn(C)c3)c2)nc1Nc1ccc2[nH]ncc2c1]

To a solution of 1-methyl-N-(3-(4-methyl-3-((1-(1-methyl-1H-pyrazole-4-carbonyl)-1H-indazol-5-yl)amino)-1H-pyrazol-1-yl)phenyl)-1H-pyrazole-4-carboxamide (256 mg, 492 μmol) in EtOH (5 mL) was added K₂CO₃ (256 mg, 1.85 mmol) and H₂O (2 mL). The mixture was stirred at r.t. for 1 hr. The reaction mixture was concentrated under reduced pressure to give N-(3-(3-((1H-indazol-5-yl)amino)-4-methyl-1H-pyrazol-1-yl)phenyl)-1-methyl-1H-pyrazole-4-carboxamide (26.6 mg, 61.27 μmol, 12.5% yield) as a yellow solid. LC-MS (ES⁺, Method A), 0.55 min, m/z 413.2 [M+H]⁺. ¹H NMR (400 MHz, DMSO-d₆) δ 12.78 (s, 1H), 9.96 (s, 1H), 8.35 (s, 1H), 8.19 (d, J=8.0 Hz, 2H), 8.07 (d, J=10.4 Hz, 2H), 7.99-7.95 (m, 2H), 7.55-7.48 (m, 1H), 7.47-7.43 (m, 1H), 7.40-7.37 (m, 3H), 3.92, (s, 3H), 2.08 (d, J=7.6 Hz, 6H).

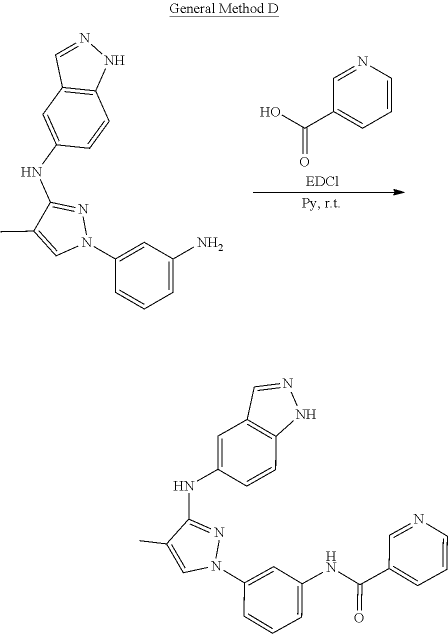 [SMILES: Cc1cn(-c2cccc(N)c2)nc1Nc1ccc2[nH]ncc2c1 ; O=C(O)c1cccnc1 ; Cc1cn(-c2cccc(NC(=O)c3cccnc3)c2)nc1Nc1ccc2[nH]ncc2c1]

### Example 4: N-(3-(3-((1H-indazol-5-yl)amino)-4-methyl-1H-pyrazol-1-yl)phenyl)nicotinamide

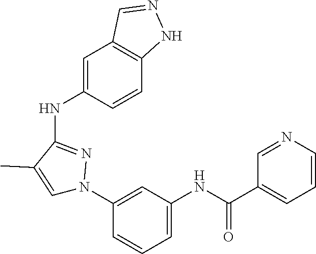 [SMILES: Cc1cn(-c2cccc(NC(=O)c3cccnc3)c2)nc1Nc1ccc2[nH]ncc2c1]

To a solution of N-(1-(3-aminophenyl)-4-methyl-1H-pyrazol-3-yl)-1H-indazol-5-amine (150 mg, 492.86 μmol) and nicotinic acid (91.0 mg, 739.28 μmol) in Py (10 mL) was added EDCI (236.2 mg, 1.23 mmol). The mixture was stirred at r.t. for 16 hr. The residue was partitioned between water (20 mL) and EtOAc (20 mL). The organic layer was separated and the aqueous layer was extracted with EtOAc (2×20 mL). The combined organics were washed with washed with brine (60 mL), dried over sodium sulfate, filtered and the solvent removed under reduced pressure to give a residue, which was purified by preparative HPLC (30-80% MeCN in H₂O) to give N-(3-(3-((1H-indazol-5-yl)amino)-4-methyl-1H-pyrazol-1-yl)phenyl)nicotinamide (34.8 mg, 82.4 μmol, 16.7% yield) as a yellow solid. LC-MS (ES⁺, Method A), 0.53 min, m/z 410.1 [M+H]⁺. ¹H NMR (400 MHz, DMSO-d₆) δ 12.77 (s, 1H) 10.59 (s, 1H) 9.16 (d, J=1.6 Hz, 1H), 8.79-8.78 (m, 1H), 8.35 (d, J=7.6 Hz, 1H), 8.30 (s, 1H), 8.22 (s, 1H), 8.11 (s, 1H), 8.01 (s, 1H), 7.94 (s, 1H), 7.60-7.59 (m, 2H), 7.48-7.47 (m, 2H), 7.42-7.41 (m, 2H), 2.10 (s, 3H).

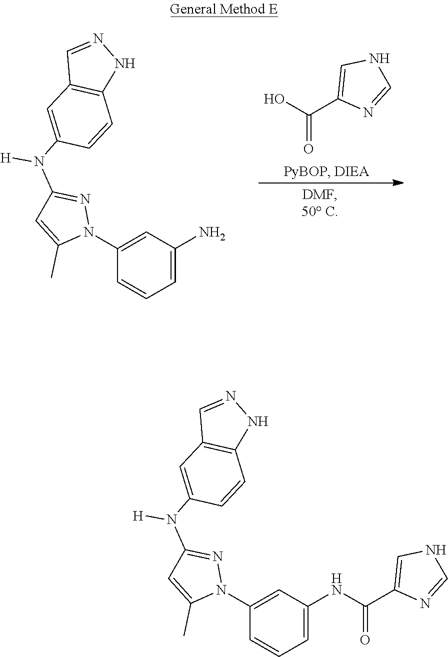 [SMILES: Cc1cc(Nc2ccc3[nH]ncc3c2)nn1-c1cccc(N)c1 ; O=C(O)c1c[nH]cn1 ; Cc1cc(Nc2ccc3[nH]ncc3c2)nn1-c1cccc(NC(=O)c2c[nH]cn2)c1]

### Example 5: N-(3-(3-((1H-indazol-5-yl)amino)-5-methyl-1H-pyrazol-1-yl)phenyl)-1-methyl-1H-pyrazole-4-carboxamide

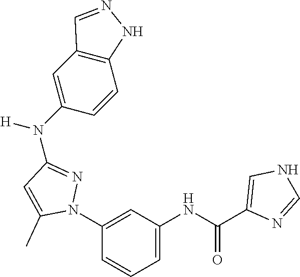 [SMILES: Cc1cc(Nc2ccc3[nH]ncc3c2)nn1-c1cccc(NC(=O)c2c[nH]cn2)c1]

To a solution of 1H-imidazole-4-carboxylic acid (40.5 mg, 361 μmol) and N-[1-(3-aminophenyl)-5-methyl-pyrazol-3-yl]-1H-indazol-5-amine (110 mg, 361 μmol) in DMF (1 mL) was NH added DIEA (12.74 mg, 98.6 μmol) and PyBOP (34.2 mg, 65.7 μmol) at r.t., and then the mixture was stirred at 50° C. for 12 hr. The reaction mixture was poured into water (20 mL), filtered by filter paper. The filter cake was washed by MeOH (10 mL), and the filter layers was concentrated under reduced pressure to give a residue. The crude product was purified by preparative HPLC (35-65% MeCN in H₂O) to give N-(3-(3-((1H-indazol-5-yl)amino)-5-methyl-1H-pyrazol-1-yl)phenyl)-1H-imidazole-4-carboxamide (50 mg, 109 μmol, 30% yield) as a yellow solid. LC-MS (ES+, Method A), 0.35 min, m/z 399.2 [M+H]⁺. ¹H NMR (400 MHz, DMSO-d6) δ 11.11 (s, 1H), 9.19 (s, 1H), 8.67 (s, 1H), 8.13 (t, J=2.0 Hz, 1H), 8.02 (d, J=1.6 Hz, 1H), 7.93 (s, 1H), 7.78 (dd, J=1.2, 8.2 Hz, 1H), 7.51 (t, J=8.0 Hz, 1H), 7.41-7.36 (m, 2H), 7.28 (dd, J=2.0, 8.8 Hz, 1H), 5.92 (s, 1H), 2.40 (s, 3H).

Compounds prepared in a similar manner to that set out above are given below in Table 4:

| Example | Structure | Method | LCMS | ¹H NMR |
|---|---|---|---|---|
| 6 | 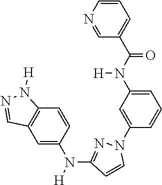 [SMILES: O=C(Nc1cccc(-n2ccc(Nc3ccc4[nH]ncc4c3)n2)c1)c1cccnc1] | A | LC-MS (ES⁺, Method A), 0.83 min, m/z 396.3 [M + H]⁺ | ¹H NMR (400 MHz, DMSO-d₆) δ 12.90-12.72 (m, 1H), 10.63 (s, 1H), 9.16 (d, J = 1.6 Hz, 1H), 8.79 (dd, J = 1.6, 4.8 Hz, 1H), 8.78- 8.75 (m, 1H), 8.37-8.33 (m, 2H), 8.29 (d, J = 2.4Hz, 1H), 8.16 (s, 1H), 8.14 (d, J = 1.2 Hz, 1H), 7.94 (s, 1H), 7.65-7.59 (m, 2H), 7.53-7.54 (m, 1H), 7.48-7.42 (m, 2H), 7.31 (dd, J = 2.0, 8.8 Hz, 1H), 6.12 (d, J = 2.4 Hz, 1H) |
| 7 | 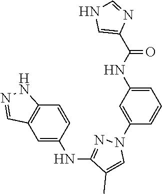 [SMILES: Cc1cn(-c2cccc(NC(=O)c3c[nH]cn3)c2)nc1Nc1ccc2[nH]ncc2c1] | C | LC-MS (ES⁺, Method A), 0.48 min, m/z 399.1 [M + H]⁺ | ¹H NMR (400 MHz, DMSO-d₆) δ 10.54-10.44 (n, 1H) 8.79-8.74 (m, 1H) 8.23 ( d, J = 2.4 Hz, 3H) 8.12 (s, 1H), 8.05-7.99 (m, 1H), 7.96 (s, 1H), 7.59 (d, J = 7.6 Hz, 1H), 7.50-7.46 (m, 2H), 7.44- 7.41 (m, 2H), 2.10 (s, 3 H) |
| 8 | 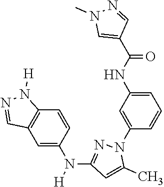 [SMILES: Cc1cc(Nc2ccc3[nH]ncc3c2)nn1-c1cccc(NC(=O)c2cnn(C)c2)c1] | D | LC-MS (ES⁺, Method A), 0.41 min, m/z 413.2 [M + H]⁺ | ¹H NMR (400 MHz, DMSO-d₆) δ 9.98 (s, 1H), 8.50 (s, 1H), 8.34 (s, 1H), 8.04 (s, 1H), 8.01-7.98 (m, 2H), 7.89 (s, 1H), 7.69-7.68 (m, 1H), 7.44 (t, J = 8.4 Hz, 1H), 7.39 (d, J = 8.8 Hz, 1H), 7.28- 7.25 (m, 2H), 5.91 (s, 1H), 3.90 (s, 3H), 2.37 (s, 3H), exchangeable NH not seen. |
| 9 | 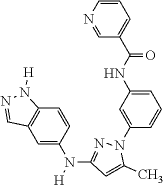 [SMILES: Cc1cc(Nc2ccc3[nH]ncc3c2)nn1-c1cccc(NC(=O)c2cccnc2)c1] | D | LC-MS (ES⁺, Method A), 0.38 min, m/z 410.2 [M + H]⁺ | ¹H NMR (400 MHz, DMSO-d₆) δ 10.87 (s, 1H), 9.30 (d, J = 1.6 Hz, 1H), 8.91 (dd, J = 5.2 Hz, 1.2 Hz, 1H), 8.64 (d, J = 8.0 Hz, 1H), 8.10 (t, J = 2.0 Hz, 1H), 8.01 (d, J = 1.6 Hz, 1H), 7.91 (s, 1H), 7.90-7.80 (m, 1H), 7.78 (d, J = 8.8 Hz, 1H), 7.51 (t, J = 8.0 Hz, 1H), 7.41-7.39 (m, 2H), 7.29-7.24 (m, 1H), 5.93 (s, 1H), 2.39 (s, 3H), exchangeable 2 × NH not seen. |

General route for the synthesis of examples 10-15:

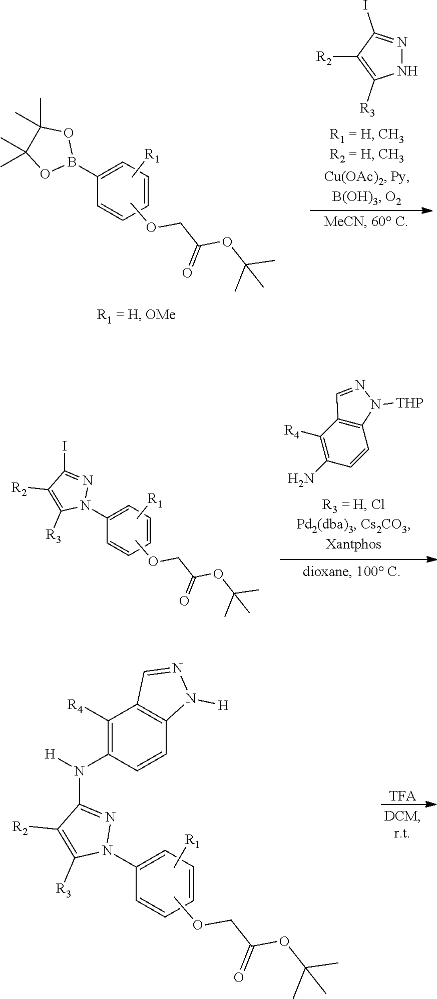

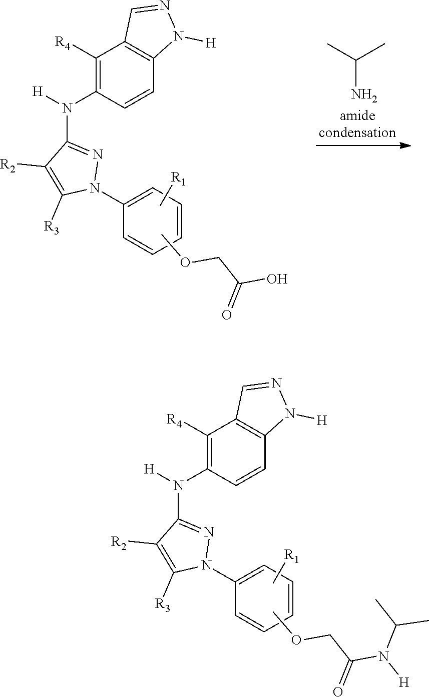

### Step 1: tert-butyl 2-(4-(3-iodo-1H-pyrazol-1-yl)phenoxy)acetate

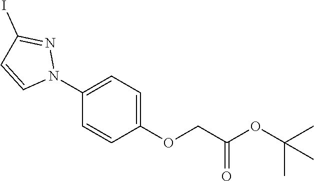 [SMILES: CC(C)(C)OC(=O)COc1ccc(-n2ccc(I)n2)cc1]

To a mixture of tert-butyl 2-(4-(4,4,5,5-tetramethyl-1,3,2-dioxaborolan-2-yl)phenoxy)acetate (1 g, 2.99 mmol), 3-iodo-I 1H-pyrazole (638.42 mg, 3.29 mmol) in MeCN (20 mL) was added “˜boric acid (370 mg, 5.98 mmol), Py (473.4 mg, 5.98 mmol), 4 Å MS (1 g, 2.99 mmol) and Cu(OAc)₂ (815.2 mg, 4.49 mmol) at r.t. The reaction was bubbled with O₂ and stirred at 60° C. under O₂ (15 Psi) for 16 hr. The reaction mixture was cooled to r.t., and EtOAc (100 mL) was added. The mixture was filtered through a pad of Celite, and the mother liquid was concentrated to give a residue. The residue was purified by flash silica gel chromatography eluting with 0-30% EtOAc in Pet. Ether to give tert-butyl 2-(4-(3-iodo-1H-pyrazol-1-yl)phenoxy)acetate (750 mg, 1.82 mmol) as a colorless oil. LC-MS (ES+, Method A), 1.07 min, m/z 401.0 [M+H]⁺.

### Step 2: tert-butyl 2-(4-(3-((1-(tetrahydro-2H-pyran-2-yl)-1H-indazol-5-yl)amino)-1H-pyrazol-1-yl)phenoxy)acetate

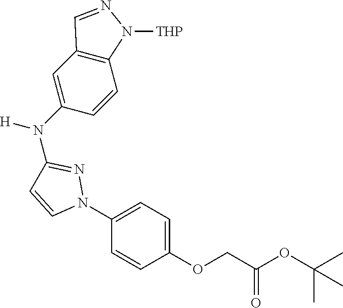

To a mixture of tert-butyl 2-(4-(3-iodo-1H-pyrazol-1-N-TH yl)phenoxy)acetate (280 mg, 678.64 μmol) and 1-tetrahydropyran-2-ylindazol-5-amine (162.2 mg, 746.51 μmol) in dioxane (5 mL) was added XPhos (64.7 mg, 135.73 μmol), Cs₂CO₃ (442.2 mg, 1.36 mmol) and Pd₂(dba)₃ (62.1 mg, 67.86 μmol) at r.t. under N₂. The suspension was degassed under reduced pressure and purged with N₂ for 5 mins. The mixture was heated to 100° C. and stirred for 16 hr. The reaction mixture was cooled to r.t. and EtOAc (100 mL) and water (100 mL) were added. The organic layer was separated, and the water phase extracted with EtOAc (3×100 mL). The combined organic phase was washed with brine (20 mL), dried with anhydrous sodium sulfate, filtered and concentrated under reduced pressure. The residue was purified by flash silica gel chromatography eluting with 15-40% EtOAc in Pet. Ether to give tert-butyl 2-(4-(3-((1-(tetrahydro-2H-pyran-2-yl)-1H-indazol-5-yl)amino)-1H-pyrazol-1-yl)phenoxy)acetate (150 mg, 300.27 μmol, 44% yield) as colorless oil. LC-MS (ES⁺, Method A), 1.00 min, m/z 490.3 [M+H]+*.

### Step 3: 2-(4-(3-((1H-indazol-5-yl)amino)-1H-pyrazol-1-yl)phenoxy)acetic acid

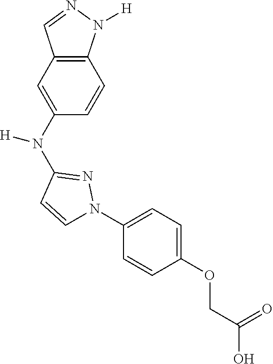 [SMILES: O=C(O)COc1ccc(-n2ccc(Nc3ccc4[nH]ncc4c3)n2)cc1]

To a mixture of tert-butyl 2-(4-(3-((1-(tetrahydro-2H-pyran-2-yl)-1H-indazol-5-yl)amino)-1H-pyrazol-1-yl)phenoxy)acetate (230 mg, 469.80 μmol) in DCM (5 mL) was added TFA (3 mL) in one portion at 0° C. The mixture was stirred at 25° C. for 1 hour. The reaction mixture was concentrated to give 2-(4-(3-((1H-indazol-5-yl)amino)-1H-pyrazol-1-yl)phenoxy)acetic acid (230 mg, 658.38 μmol) as yellow oil. LC-MS (ES⁺, Method A), 0.83 min, m/z 350.1 [M+H]⁺.

Compounds prepared in a similar manner to that described above are given below in Table 5:

| Example | Structure | Method | LCMS | ¹H NMR |
|---|---|---|---|---|
| 10 | 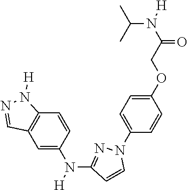 [SMILES: CC(C)NC(=O)COc1ccc(-n2ccc(Nc3ccc4[nH]ncc4c3)n2)cc1] | A | LC-MS (ES⁺, Method C), 0.92 min, m/z 391.2 [M + H]⁺ | ¹H NMR (400 MHz, DMSO-d₆) δ 12.76 (s, 1H), 8.64 (s, 1H), 8.23 (d, J = 2.4 Hz, 1H), 8.04 (d, J = 1.6 Hz, 1H), 7.93-7.89 (m, 2H), 7.74-7.72 (m, 2H), 7.43-7.41 (m, 2H), 7.29 (dd, J = 1.6, 8.8 Hz, 1H), 7.07 (dd, J = 2.4, 7.2 Hz, 1H), 6.05 (d, J = 2.8 Hz, 1H), 4.47 (s, 2H), 4.01-3.92 (m, 1H), 1.11 (d, J = 6.4 Hz, 6H) |
| 11 | 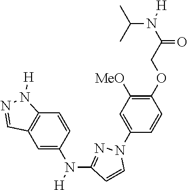 [SMILES: COc1cc(-n2ccc(Nc3ccc4[nH]ncc4c3)n2)ccc1OCC(=O)NC(C)C] | A | LC-MS (ES⁺, Method A), 0.52 min, m/z 421.2 [M + H]⁺ | ¹H NMR (400 MHz, DMSO-d₆) δ ppm 8.66 (s, 1H), 8.31 (d, J = 2.8 Hz, 1 H), 8.02 (d, J = 1.2 Hz, 1H), 7.94 (d, J = 0.8 Hz, 1H), 7.76 (d, J = 7.6 Hz, 1H), 7.40-7.45 (m, 1H), 7.24-7.35 (m, 1H), 7.05 (d, J = 8.8 Hz, 1H) 6.08 (d, J = 2.8 Hz, 1H) 4.44 (s, 2H) 3.92 (s, 4H) 1.10 (d, J = 6.4 Hz, 6H) |
| 12 | 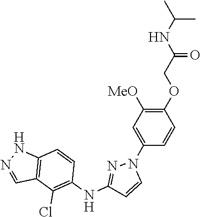 [SMILES: COc1cc(-n2ccc(Nc3ccc4[nH]ncc4c3Cl)n2)ccc1OCC(=O)NC(C)C] | | LC-MS (ES⁺, Method A), 0.58 min, m/z 455.3 [M + H]⁺ | ¹H NMR (400 MHz, MeOD-d₄) δ 8.07-8.04 (m, 1H), 8.03- 7.97 (m, 2H), 7.49-7.43 (m, 1H), 7.42-7.39 (m, 1H), 7.24- 7.18 (m, 1H), 7.11-7.05 (m, 1H), 6.21-6.16 (m, 1H), 4.54- 4.45 (m, 2H), 4.15-4.04 (m, 1H), 3.99-3.94 (m, 3H), 1.24- 1.17 (m, 6H) |
| 13 | 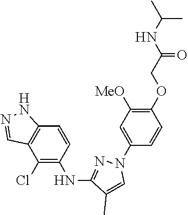 [SMILES: COc1cc(-n2cc(C)c(Nc3ccc4[nH]ncc4c3Cl)n2)ccc1OCC(=O)NC(C)C] | A | LC-MS (ES⁺, Method A), 0.66 min, m/z 469.1 [M + H]⁺. | ¹H NMR (400 MHz, DMSO-d₆) δ 13.17 (s, 1H), 8.21 (s, 1H), 7.98 (s, 1H), 7.74 (d, J = 7.6 Hz, 1H), 7.60 (d, J = 8.8 Hz, 1H), 7.42 (d, J = 8.8 Hz, 1H), 7.34 (d, J = 2.4 Hz, 1H), 7.20-7.01 (m, 2H), 7.00 (d, J = 8.8 Hz, 1H), 4.42 (s, 2H), 3.95-3.89 (m, 1H), 3.87 (s, 3H), 1.98 (s, 3H), 1.09 (d, J = 6.4 Hz, 6H) |
| 14 | 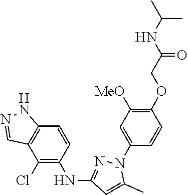 [SMILES: COc1cc(-n2nc(Nc3ccc4[nH]ncc4c3Cl)cc2C)ccc1OCC(=O)NC(C)C] | A | LC-MS (ES⁺, Method A), 0.47 min, m/z 469.8 [M + H]⁺ | ¹H NMR (400 MHz, MeOD-d₄) δ 8.01 (s, 1H), 7.77 (d, J = 8.8 Hz, 1H) 7.46-7.44 (m, 1H), 7.18 (d, J = 2.4 Hz, 1 H), 7.13-7.11 (m, 1H), 7.04-7.03 (m, 1H), 5.98 (s, 1H), 4.55 (s, 2H), 4.10-4.07 (m, 1H), 3.93 (s, 3H), 2.30 (s, 3H), 1.20 (d, J 6.4 Hz, 6H) |
| 15 | 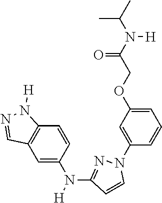 [SMILES: CC(C)NC(=O)COc1cccc(-n2ccc(Nc3ccc4[nH]ncc4c3)n2)c1] | A | LC-MS (ES⁺, Method A), 0.89 min, m/z 391.4 [M + H]⁺ | ¹H NMR (400 MHz, DMSO-d₆) δ 12.78 (s, 1H), 8.75 (s, 1H), 8.35 (d, J = 2.8 Hz, 1H), 8.11 (d, J = 1.2 Hz, 1H), 8.02-7.93 (m, 2H), 7.45-7.35 (m, 4H), 7.31-7.32 (m, 1H), 6.78 (d, J = 8.0 Hz, 1H), 6.09 (d, J = 2.4 Hz, 1H), 4.54 (s, 2H), 4.04-3.94 (m, 1H), 1.10 (d, J = 6.8 Hz, 6H) |

General route for the synthesis of examples 16-20:

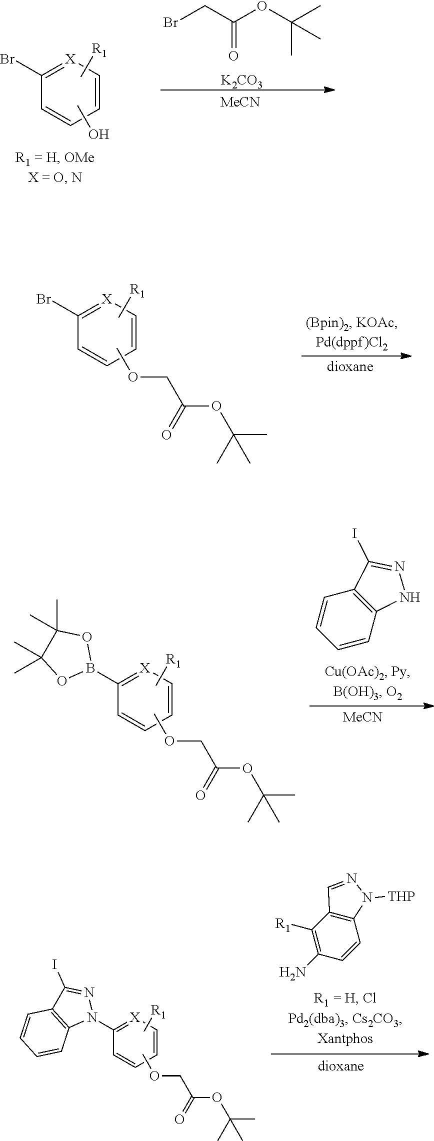

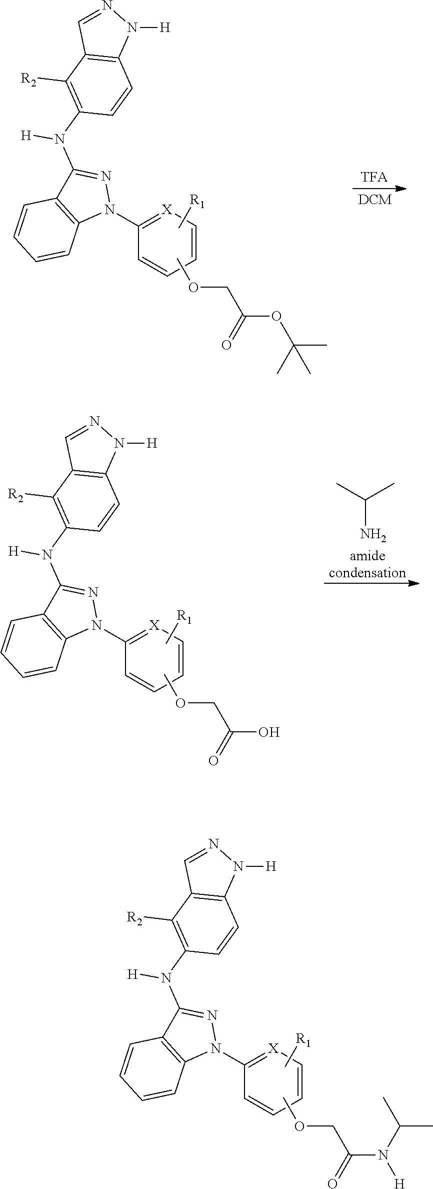

### Step 1: tert-butyl 2-(4-bromophenoxy)acetate

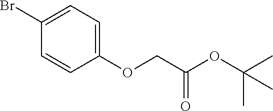 [SMILES: CC(C)(C)OC(=O)COc1ccc(Br)cc1]

To a solution of 4-bromophenol (30 g, 173.40 mmol), tert-butyl 2-bromoacetate (43.97 g, 225.42 mmol) in MeCN (350 mL) was added K₂CO₃ (47.93 g, 346.80 mmol). The mixture was stirred at 80° C. for 16 hr. The reaction was cooled to room temperature and filtered. The solid was washed with EtOAc (200 mL). The filtrate was concentrated. The residue was purified by reversed phase column (basic conditions) to give tert-butyl 2-(4-bromophenoxy)acetate (41 g, 142.78 mmol, 82% yield) as brown oil.

### Step 2: tert-butyl 2-(4-(4,4,5,5-tetramethyl-1,3,2-dioxaborolan-2-yl)phenoxy)acetate

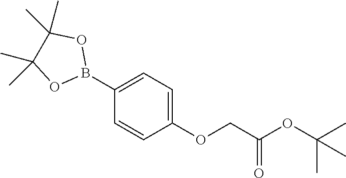 [SMILES: CC(C)(C)OC(=O)COc1ccc(B2OC(C)(C)C(C)(C)O2)cc1]

A mixture of tert-butyl 2-(4-bromophenoxy)acetate (41 g, 142.78 mmol), 4,4,5,5-tetramethyl-2-(4,4,5,5-tetramethyl-1,3,2-dioxaborolan-2-yl)-1,3,2-dioxaborolane (43.5 g, 171.34 mmol,) and KOAc (28 g, 285.57 mmol) in dioxane (500 mL) was degassed with N₂ for 5 min, then Pd(dppf)Cl₂ (5.22 g, 7.14 mmol) was added and the reaction mixture was degassed with N₂ for another 5 min. The reaction was stirred at 90° C. for 16 hr. The reaction was cooled to room temperature and concentrated to remove the solvent to give a residue. The residue was purified by column chromatography eluting with 0-10% EtOAc in Pet. Ether to give to give a crude product. The crude product was re-purified by reversed phase column (basic conditions) to give tert-butyl 2-(4-(4,4,5,5-tetramethyl-1,3,2-dioxaborolan-2-yl)phenoxy)acetate (30.3 g, 90.66 mmol, 64% yield) as a white solid.

### Step 3: tert-butyl 2-(4-(3-iodo-1H-indazol-1-yl)phenoxy)acetate

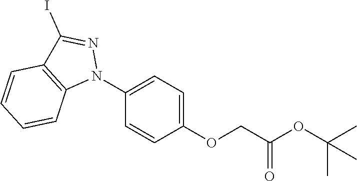 [SMILES: CC(C)(C)OC(=O)COc1ccc(-n2nc(I)c3ccccc32)cc1]

To a mixture of tert-butyl 2-(4-(4,4,5,5-tetramethyl-1,3,2-dioxaborolan-2-yl)phenoxy)acetate (1 g, 2.99 mmol), 3-iodo-1H-indazole (803 mg, 3.29 mmol) in MeCN (20 mL) was 0 added Boric acid (370 mg, 5.98 mmol), Py (473 mg, 5.98 mmol), 4 Å MS (1 g, 2.99 mmol) and Cu(OAc)₂ (815 mg, 4.49 mmol) at r.t.. The reaction was bubbled with O₂ and stirred at 60° C. under O₂ (15 Psi) for 16 hr. The reaction mixture was cooled to r.t., and EtOAc (100 mL) was added. The mixture was filtered through a pad of Celite, and the mother liquid was concentrated to give a residue. The residue was purified by flash silica gel chromatography eluting with 0-30% EtOAc in Pet. Ether to give tert-butyl 2-(4-(3-iodo-1H-indazol-1-yl)phenoxy)acetate (930 mg, 2.00 mmol) was obtained as a colorless oil. LC-MS (ES⁺, Method A), 1.13 min, m/z 451.2 [M+H]⁺.

### Step 4: tert-butyl 2-(4-(3-((1-(tetrahydro-2H-pyran-2-yl)-1H-indazol-5-yl)amino)-1H-indazol-1-yl)phenoxy)acetate

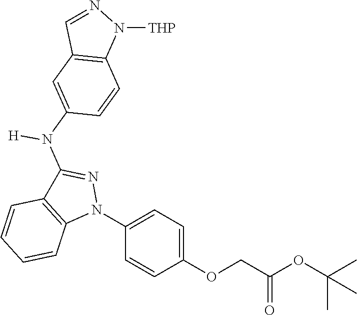

To a mixture of tert-butyl 2-(4-(3-iodo-1H-indazol-1-yl)phenoxy)acetate (930 mg, 2.00 mmol) and 1-(tetrahydro-2H-HN pyran-2-yl)-1H-indazol-5-amine (440 mg, 2.00 mmol) in dioxane (12 mL) was added Xantphos (116 mg, 200.35 μmol), Cs₂CO₃ (1.31 g, 4.01 mmol) and Pd₂(dba)₃ (183 mg, 200.35 μmol) at r.t. under N₂. The suspension was degassed under reduced pressure and purged with N₂ for 5 min. The mixture was heated to 105° C. and stirred for 16 hr. The reaction mixture was poured into water (100 mL) and extracted with EtOAc (100 mL×3), and the combined organic phase was washed with brine (100 mL), dried with anhydrous sodium sulfate, filtered and concentrated under reduced pressure. The residue was purified by flash silica gel chromatography eluting with 0-16% EtOAc in Pet. Ether to give tert-butyl 2-(4-(3-((1-(tetrahydro-2H-pyran-2-yl)-1H-indazol-5-yl)amino)-1H-indazol-1-yl)phenoxy)acetate (560 mg, 985.87 μmol, 49% yield) as a colorless oil. LC-MS (ES+, Method A), 1.16 min, m/z 540.3 [M+H]⁺.

### Step 5: 2-(4-(3-((1H-indazol-5-yl)amino)-1H-indazol-1-yl)phenoxy)acetic acid

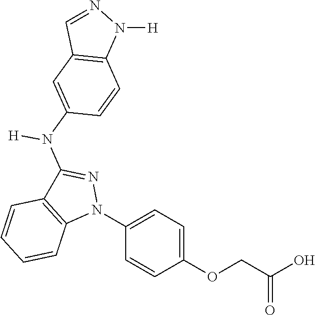 [SMILES: O=C(O)COc1ccc(-n2nc(Nc3ccc4[nH]ncc4c3)c3ccccc32)cc1]

To a solution of tert-butyl 2-(4-(3-((1-(tetrahydro-2H-pyran-2-yl)-1H-indazol-5-yl)amino)-1H-indazol-1-yl)phenoxy)acetate (200 mg, 352.10 μmol) in DCM (8 mL) was added TFA (6.16 g, 54.02 mmol) at 0° C. under N₂. The mixture was stirred at r.t. for 2 hr. The reaction mixture was concentrated to give 2-(4-(3-((1H-indazol-5-yl)amino)-1H-indazol-1-yl)phenoxy)acetic acid (140 mg, 350.52 μmol, 99% yield) as a yellow oil. LC-MS (ES⁺, Method A), 0.92 min, m/z 400.1 [M+H]⁺.

Compounds prepared in a similar manner to that described above are given below in Table 6:

| Example | Structure | Method | LCMS | ¹H NMR |
|---|---|---|---|---|
| 16 | 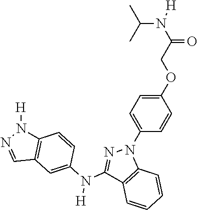 [SMILES: CC(C)NC(=O)COc1ccc(-n2nc(Nc3ccc4[nH]ncc4c3)c3ccccc32)cc1] | A | LC-MS (ES⁺, Method A), 0.95 min, m/z 441.4 [M + H]⁺ | ¹H NMR (400 MHz, DMSO-d₆) δ 12.87 ( s, 1H), 9.10 ″(s, 1H), 8.41 (s, 1H), 8.16 (d, J = 8.0 Hz, 1H), 8.02- 7.94 (m, 2H), 7.73-7.67 (m, 3H), 7.62-7.57 (m, 1H), 7.53-7.44 (m, 2H), 7.21-7.14 (m, 3H), 4.52 (s, 2H), 4.04-3.93 (m, 1H), 1.12 (d, J = 6.4 Hz, 6H) |
| 17 | 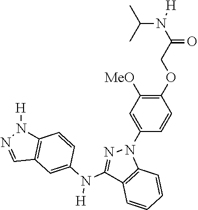 [SMILES: COc1cc(-n2nc(Nc3ccc4[nH]ncc4c3)c3ccccc32)ccc1OCC(=O)NC(C)C] | A | LC-MS (ES⁺, Method A), 0.68 min, m/z 471.0 [M + H]⁺ | ¹H NMR (400 MHz, DMSO-d₆) δ 12.89-12.79 (m, 1H), 9.12 (s, 1H), 8.42 (d, J = 1.6 Hz, 1H), 8.15 (d, J = 8.0 Hz, 1H), 7.98 (s, 1H), 7.76 (d, J = 8.4 Hz, 2H), 7.57 (d, J = 2.0 Hz, 1H), 7.50 (d, J = 9.6 Hz, 2H), 7.39 (d, J = 2.4 Hz, 1H), 7.31-7.25 (m, 1H), 7.23-7.18 (m, 1H), 7.13 (d, J = 8.8 Hz, 1H), 4.50 (s, 2H), 3.94 (s, 3H) 1.12 (d, J = 6.4 Hz, 6H) |
| 18 | 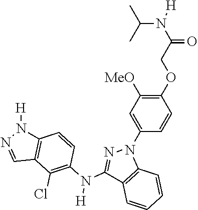 [SMILES: COc1cc(-n2nc(Nc3ccc4[nH]ncc4c3Cl)c3ccccc32)ccc1OCC(=O)NC(C)C] | E | LC-MS (ES⁺, Method A), 0.65 min, m/z 505.1 [M + H]⁺. | ¹H NMR (400 MHz, DMSO-d₆) δ 13.47-13.17 (m, 1H), 8.37 (s, 1H), 8.06 (s, 1H), 7.94 (d, J = 8.8 Hz, 1H), 7.89-7.82 (m, 1H), 7.81-7.76 (m, 1H), 7.75-7.71 (m, 1H), 7.55-7.50 (m, 1H), 7.49-7.43 (m, 1H), 7.30- 7.24 (m, 1H), 7.20-7.12 (m, 2H), 7.11-7.03 (m, 1H), 4.52-4.43 (m, 2H), 3.95-3.95 (m, 1H), 3.99-3.89 (m, 1H), 3.89-3.83 (m, 3H), 1.20-1.00 (m, 7H) |
| 19 | 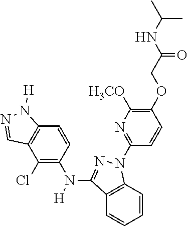 [SMILES: COc1nc(-n2nc(Nc3ccc4[nH]ncc4c3Cl)c3ccccc32)ccc1OCC(=O)NC(C)C] | A | LC-MS (ES⁺, Method A), 0.53 min, m/z 506.2 [M + H]⁺. | ¹H NMR (400 MHz, DMSO-d₆) δ 13.35 (s, 1H), 8.61 (d, J = 8.4 Hz, 1H), 8.57 (s, 1H), 8.09 (s, 1H), 8.01 (d, J = 8.0 Hz, 1H), 7.92 (d, J = 8.8 Hz, 1H), 7.81 (d, J = 7.6 Hz, 1H), 7.58-7.54 (m, 2H), 7.41 (d, J = 8.4 Hz, 1H), 7.23 (t, J = 7.6 Hz, 1H), 7.14 (d, J = 8.4 Hz, 1H), 4.45 (s, 2H), 4.10 (s, 3H), 3.96-3.89 (m, 1H), 1.08 (d, J = 6.8 Hz, 6H). |
| 20 | 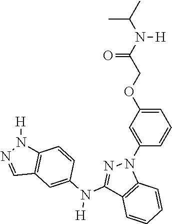 [SMILES: CC(C)NC(=O)COc1cccc(-n2nc(Nc3ccc4[nH]ncc4c3)c3ccccc32)c1] | A | LC-MS (ES⁺, Method A), 0.96 min, m/z 441.4 [M + H]⁺ | ¹H NMR (400 MHz, DMSO-d₆) δ 12.86 (s, 1H), 9.14 (s, 1H), 8.43 (d, J = 1.2 Hz, 1H), 8.19 (d, J = 8.0 Hz, 1H), 8.04 (s, 1H), 7.98 (d, J = 8.0 Hz, 1H), 7.85 (d, J = 8.8 Hz, 1H), 7.64-7.60 (m, 1H), 7.54-7.48 (m, 3H), 7.47-7.39 (m, 3H), 7.24 (t, J = 7.6 Hz, 1H), 6.88 (d, J = 7.6 Hz, 1H), 4.58 (s, 2H), 4.04-3.95 (m, 1H), 1.10 (d, J = 6.8 Hz, 6H). |

### General Route or the Synthesis of Examples 21-32:

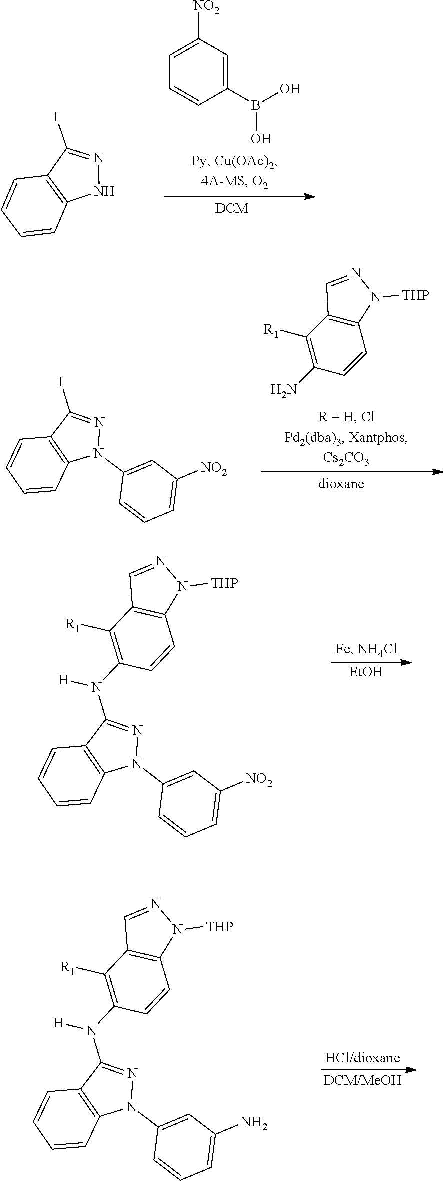

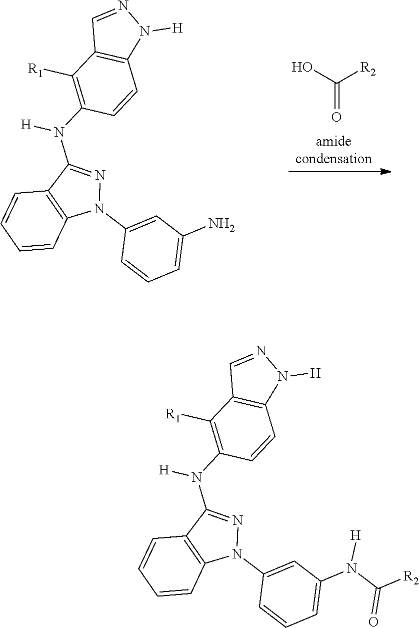

### Step 1: 3-iodo-1-(3-nitrophenyl)-1H-indazole

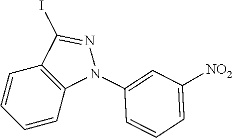 [SMILES: O=[N+]([O-])c1cccc(-n2nc(I)c3ccccc32)c1]

To a mixture of (3-nitrophenyl)boronic acid (4.10 g, 24.6 mmol) and 3-iodo-1H-indazole (3 g, 12.3 mmol) in DCM (8 mL) was added 4 Å MS (0.8 g, 1.23 mmol), Py (1.94 g, 24.6 mmol) and Cu(OAc)₂ (3.35 g, 18.4 mmol) at r.t.. The mixture was stirred at r.t. for 16 hours under O₂ (15 Psi). The reaction mixture was poured into EtOAc (500 mL) and filtered to remove 4 Å MS and Copper salt. the mother liquid was concentrated to give a residue the residue was purified by flash silica gel chromatography eluting with 0-10% EtOAc in Pet. Ether to give to give 3-iodo-1-(3-nitrophenyl)-1H-indazole (3.95 g, 10.3 mmol, 84% yield) as a yellow solid. LC-MS (ES⁺, Method A), 1.07 min, m/z 366.2 [M+H]⁺.

### Step 2: 1-(3-nitrophenyl)-N-(1-(tetrahydro-2H-pyran-2-yl)-1H-indazol-5-yl)-1H-indazol-3-amine

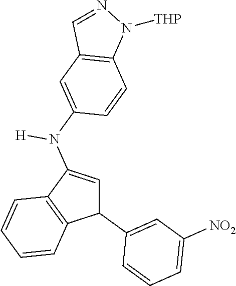

To a mixture of 3-iodo-1-(3-nitrophenyl)-1H-indazole (650 mg, 1.76 mmol) and 1-(tetrahydro-2H-pyran-2-yl)-1H-indazol-5-amine (423 mg, 1.85 mmol) in dioxane (12 mL) was added Xantphos (102 mg, 176 μmol), Cs₂CO₃ (1.15 g, 3.52 mmol) and Pd₂(dba)₃ (161 mg, 176 μmol) at r.t under N₂. The suspension was degassed under reduced pressure and purged with N₂ for 5 mins. The mixture was heated to 105° C. and stirred for 16 hr. The reaction mixture was cooled to 20° C. and poured into water (50 mL) and EtOAc (50 mL). The organic layer was separated, and water phase was extracted with EtOAc (50 mL×2). The combined organic phase was washed with brine (50 mL), dried with anhydrous sodium sulfate, filtered and concentrated under reduced pressure. The residue was purified by flash silica gel chromatography eluting with 0-30% EtOAc in Pet. Ether to give 1-(3-nitrophenyl)-N-(1-(tetrahydro-2H-pyran-2-yl)-1H-indazol-5-yl)-1H-indazol-3-amine (720 mg, 1.58 mmol, 90% yield,) as a red oil. LC-MS (ES⁺, Method A), 1.07 min, m/z 455.2 [M+H]⁺.

### Step 3: 1-(3-aminophenyl)-N-(1-(tetrahydro-2H-pyran-2-yl)-1H-indazol-5-yl)-1H-indazol-3-amine

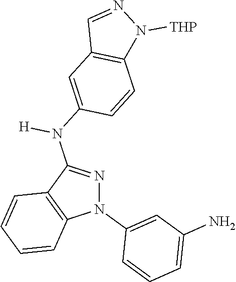

To a mixture of 1-(3-nitrophenyl)-N-(1-(tetrahydro-2H-pyran-2-yl)-1H-indazol-5-yl)-1H-indazol-3-amine (670 mg, 1.47 mmol) in EtOH (40 mL) and H₂O (8 mL) was added NH₄Cl (473 mg, 8.85 mmol) H-N at r.t. under N₂. The mixture was heated to 50° C. and Fe (453 mg, 8.11 mmol) was added. The mixture was stirred at 80° C. for 1 hr. EtOAc (100 mL) was added and the mixture was filtered through a pad of Celite. The mother liquid was concentrated to give a residue. EtOAc (100 mL) and water (100 mL) were added, and the organic layer was separated. The water phase was then extracted with EtOAc (50 mL×2), and the combined organic phase was washed with brine (100 mL), dried with anhydrous sodium sulfate, filtered and concentrated under reduced pressure to give 1-(3-aminophenyl)-N-(1-(tetrahydro-2H-pyran-2-yl)-1H-indazol-5-yl)-1H-indazol-3-amine (625 mg, 1.38 mmol, 94% yield) as a yellow solid. LC-MS (ES⁺, Method A), 0.89 min, m/z 425.2 [M+H]⁺.

### Step 4: 1-(3-aminophenyl)-N-(1H-indazol-5-yl)-1H-indazol-3-amine

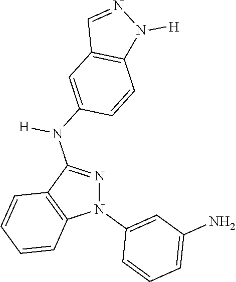 [SMILES: Nc1cccc(-n2nc(Nc3ccc4[nH]ncc4c3)c3ccccc32)c1]

To a solution of 1-(3-aminophenyl)-N-(1-(tetrahydro-2H-pyran-2-yl)-1H-indazol-5-yl)-1H-indazol-3-amine (575 mg, 1.27 mmol) in DCM (10 mL) and MeOH (10 mL) was added HCl/dioxane (4 M, H-N 11.5 mL) at r.t. under N₂. The mixture was stirred at r.t for 2 hr. The reaction mixture was concentrated to give a residue. EtOAc (50 mL) was added into the residue and stirred at r.t. for 1 hour. The mixture was filtered to give 1-(3-aminophenyl)-N-(1H-indazol-5-yl)-1H-indazol-3-amine (470 mg, 1.21 mmol, 95% yield) as a yellow solid. LC-MS (ES⁺, Method A), 0.84 min, m/z 341.2 [M+H]⁺.

General Method for synthesis of example 24:

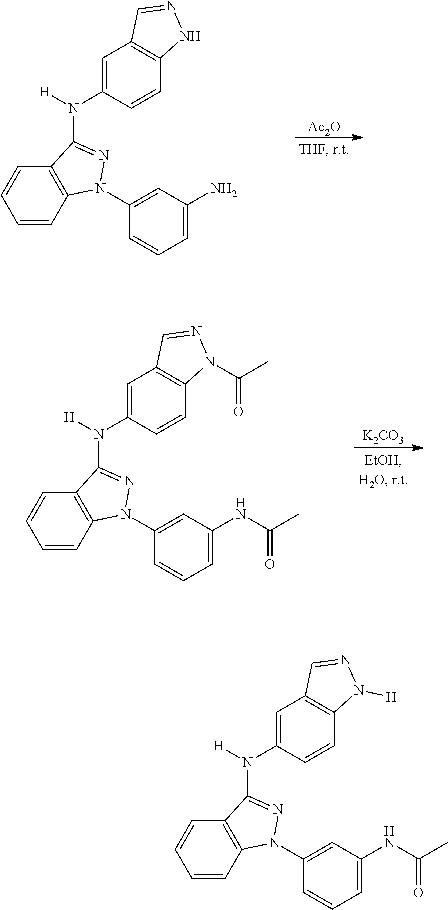 [SMILES: "Ac2O" ; Nc1cccc(-n2nc(Nc3ccc4[nH]ncc4c3)c3ccccc32)c1 ; CC(=O)Nc1cccc(-n2nc(Nc3ccc4c(cnn4C(C)=O)c3)c3ccccc32)c1 ; "K2CO3" ; CC(=O)Nc1cccc(-n2nc(Nc3ccc4[nH]ncc4c3)c3ccccc32)c1]

### Step 1: N-(3-(3-((1-acetyl-1H-indazol-5-yl)amino)-1H-indazol-1-yl)phenyl)acetamide

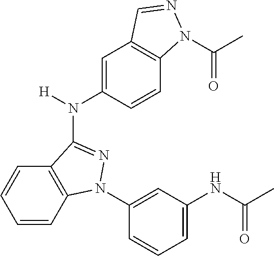 [SMILES: CC(=O)Nc1cccc(-n2nc(Nc3ccc4c(cnn4C(C)=O)c3)c3ccccc32)c1]

To a solution of 1-(3-aminophenyl)-N-(1H-indazol-5-yl)-1H-indazol-3-amine (70 mg, 205.65 μmol) in THE (2 mL) was added Ac₂O (1.14 g, 11.21 mmol) at r.t. The reaction was stirred at r.t. for 2 hr. The reaction was partitioned between H₂O (10 mL) and EtOAc (20 mL). The organic layer was separated and the aqueous layer was extracted with EtOAc (3×20 mL). The combined organics were washed with brine (2×10 mL), dried over sodium sulfate, filtered and the solvent removed under reduced pressure to give N-(3-(3-((1-acetyl-1H-indazol-5-yl)amino)-1H-indazol-1-yl)phenyl)acetamide (70 mg, crude) as a brown solid. LC-MS (ES⁺, Method A), 0.64 min, m/z 425.3 [M+H]⁺.

### Example 24: N-(3-(3-((1H-indazol-5-yl)amino)-1H-indazol-1-yl)phenyl)acetamide

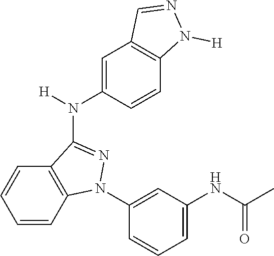 [SMILES: CC(=O)Nc1cccc(-n2nc(Nc3ccc4[nH]ncc4c3)c3ccccc32)c1]

To a solution of N-(3-(3-((1-acetyl-1H-indazol-5-yl)amino)-1H-indazol-1-yl)phenyl)acetamide (70 mg, 164.92 μmol) in EtOH (2 mL) and H₂O (1 mL) was added K₂CO₃ (70 mg, 506.49 μmol) at r.t. The reaction was stirred at r.t. for 2 hr. The reaction solvent removed under reduced pressure and the crude product was purified by preparative HPLC (30-60% MeCN in H₂O) to give N-(3-(3-((1H-indazol-5-yl)amino)-1H-indazol-1-yl)phenyl)acetamide (23.38 mg, 60.28 μmol, 37% yield) as a white solid. LC-MS (ES⁺, Method A), 0.57 min, m/z 383.3 [M+H]⁺. ¹H NMR (400 MHz, DMSO-d₆) δ 12.89 (s, 1H), 10.17 (s, 1H), 9.15 (s, 1H), 8.46 (d, J=1.6 Hz, 1H), 8.22-8.14 (m, 2H), 8.01 (s, 1H), 7.86 (d, J=8.8 Hz, 1H), 7.61 (dd, J=8.8 Hz, 2.0 Hz, 1H), 7.53-7.48 (m, 5H), 7.25 (t, J=7.6 Hz, 1H), 2.12 (s, 3H).

Compounds prepared in a similar manner to that set out above are given below in Table 7:

| Example | Structure | Method | LCMS | ¹H NMR |
|---|---|---|---|---|
| 21 | 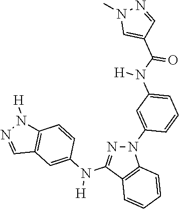 [SMILES: Cn1cc(C(=O)Nc2cccc(-n3nc(Nc4ccc5[nH]ncc5c4)c4ccccc43)c2)cn1] | A | LC-MS (ES⁺, Method A), 0.92 min, m/z 449.4 [M + H]⁺ | ¹H NMR (400 MHz, DMSO-d₆) δ 12.86 (s, 1H), 10.03 (s, 1H), 9.15 (s, 1H), 8.47 (d, J = 1.6 Hz, 1H), 8.37 (s, 1H), 8.34-8.31 (m, 1H), 8.20 (d, J = 8.0 Hz, 1H), 8.07 (s, 1H), 8.02 (s, 1H), 7.91 (d, J = 8.8 Hz, 1H), 7.65-7.60 (m, 2H), 7.57-7.49 (m, 4H), 7.25 (t, J = 7.6 Hz, 1H), 3.92 (s, 3H) |
| 22 | 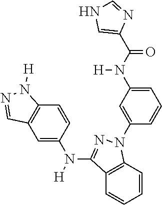 [SMILES: O=C(Nc1cccc(-n2nc(Nc3ccc4[nH]ncc4c3)c3ccccc32)c1)c1c[nH]cn1] | B | LC-MS (ES⁺, Method A), 0.92 min, m/z 435.4 [M + H]⁺ | ¹H NMR (400 MHz, DMSO-d₆) δ 12.86 (s, 1H), 12.78-12.56 (m, 1H), 10.18 (s, 1H), 9.14 (s, 1H), 8.49-8.43 (m, 2H), 8.26 (s, 1H), 8.20 (d, J = 8.0 Hz, 1H), 8.04-7.96 (m, 2H), 7.86 (s, 2H), 7.73 (m, 1H), 7.63 (dd, J = 2.0, 8.8 Hz, 1H), 7.56-7.48 (m, 4H), 7.25 (t, J = 7.6 Hz, 1H) |
| 23 | 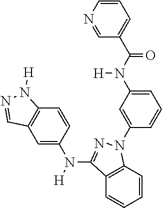 [SMILES: O=C(Nc1cccc(-n2nc(Nc3ccc4[nH]ncc4c3)c3ccccc32)c1)c1cccnc1] | A | LC-MS (ES⁺, Method A), 0.89 min, m/z 446.3 [M + H]⁺ | ¹H NMR (400 MHz, DMSO-d₆) δ 12.99-12.74 (m, 1H), 10.67 (s, 1H), 9.22-9.13 (m, 2H), 8.80 (m, 1H), 8.50 (d, J = 1.6 Hz, 1H), 8.41-8.34 (m, 2H), 8.21 (d, J = 8.0 Hz, 1H), 8.13 (s, 1H), 8.02 (s, 1H), 7.94 (d, J = 8.8 Hz, 1H), 7.76-7.69 (m, 1H), 7.65- 7.59 (m, 3H), 7.59-7.50 (m, 3H), 7.26 (t, J = 7.6 Hz, 1H) |
| 25 | 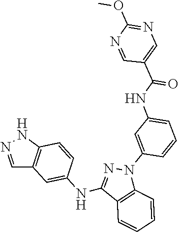 [SMILES: COc1ncc(C(=O)Nc2cccc(-n3nc(Nc4ccc5[nH]ncc5c4)c4ccccc43)c2)cn1] | D | LC-MS (ES⁺, Method A), 0.61 min, m/z 477.2 [M + H]⁺ | ¹H NMR (400 MHz, DMSO-d₆) δ 12.87 (s, 1H), δ 10.61 (s, 1H), 9.18 (s, 3H), 8.49 (s, 1H), 8.37 (s, 1H), 8.21 (d, J = 8.0 Hz, 1H), 8.02 (s, 1H), 7.93 (d, J = 8.4 Hz, 1H), 7.65-7.53 (m, 6H), 7.26 (t, J = 7.2 Hz, 1H), 4.03 (s, 3H) |
| 26 | 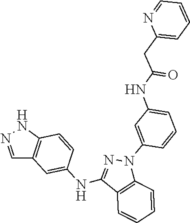 [SMILES: O=C(Cc1ccccn1)Nc1cccc(-n2nc(Nc3ccc4[nH]ncc4c3)c3ccccc32)c1] | D | LC-MS (ES⁺, Method A), 0.48 min, m/z 460.3 [M + H]⁺ | ¹H NMR (400 MHz, DMSO-d₆) δ 12.77 (s, 1H), 10.61 (s, 1H), 9.16 (s, 1H), 8.76 (d, J = 4.8 Hz, 1H), 8.49 (s, 1H), 8.30 (s, 1H), 8.26-8.13 (m, 2H), 7.92 (s, 1H), 7.86 (d, J = 8.4 Hz, 1H), 7.80 (d, J = 8.0 Hz, 1H), 7.70-7.61 (m, 1H), 7.60-7.49 (m, 5H), 7.45-7.42 (m, 1H), 7.29-7.15 (m, 1H), 4.15 (s, 2H) |
| 27 | 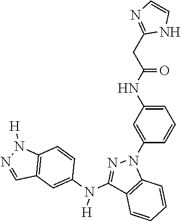 [SMILES: O=C(Cc1ncc[nH]1)Nc1cccc(-n2nc(Nc3ccc4[nH]ncc4c3)c3ccccc32)c1] | D | LC-MS (ES⁺, Method A), 0.46 min, m/z 449.3 [M + H]⁺ | ¹H NMR (400 MHz, DMSO-d₆) δ 14.19 (s, 2H), 10.74 (s, 1H), 9.18 (s, 1H), 8.51 (s, 1H), 8.29 (s, 1H), 8.21 (d, J = 8.0 Hz, 1H), 7.94 (s, 1H), 7.87 (d, J = 8.4 Hz, 1H), 7.66 (s, 2H), 7.59-7.52 (m, 5H), 7.42 (d, J = 8.0 Hz, 1H), 7.25 (t, J 7.6 Hz, 1H), 4.28 (s, 2H) |
| 28 | 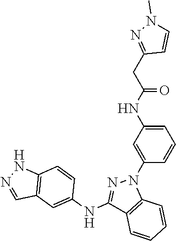 [SMILES: Cn1ccc(CC(=O)Nc2cccc(-n3nc(Nc4ccc5[nH]ncc5c4)c4ccccc43)c2)n1] | C | LC-MS (ES⁺, Method A), 0.65 min, m/z 463.1 [M + H]⁺ | ¹H NMR (400 MHz, DMSO-d₆) δ = 10.40-10.32 (m, 1H), 9.21-9.08 (m, 1H), 8.51-8.45 (m, 1H), 8.32-8.25 (m, 1H), 8.22-8.15 (m, 1H), 8.03-7.97 (m, 1H), 7.90-7.83 (m, 1H), 7.65-7.58 (m, 2H), 7.56-7.49 (m, 3H), 7.49-7.43 (m, 2H), 7.27-7.21 (m, 1H), 6.23-6.18 (m, 1H), 3.80 (s, 3H), 3.67-3.64 (m, 2H). |
| 29 | 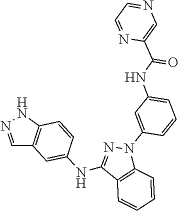 [SMILES: O=C(Nc1cccc(-n2nc(Nc3ccc4[nH]ncc4c3)c3ccccc32)c1)c1cnccn1] | D | LC-MS (ES⁺, Method A), 0.61 min, m/z 447.3 [M + H]⁺ | ¹H NMR (400 MHz, DMSO-d₆) & 12.88 (s, 1H), 11.07 (s, 1H), 9.38 (d, J = 1.2 Hz, 1H), 9.18 (s, 1H), 8.97 (d, J = 2.8 Hz, 1H), 8.87-8.83 (m, 1H), 8.58-8.46 (m, 2H), 8.22 (d, J = 8.4 Hz, 1H), 8.04-7.98 (m, 2H), 7.86 (d, J = 8.0 Hz, 1H), 7.59-7.51 (m, 5H), 7.27 (t, J = 7.2 Hz, 1H). |
| 30 | 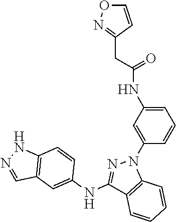 [SMILES: O=C(Cc1ccon1)Nc1cccc(-n2nc(Nc3ccc4[nH]ncc4c3)c3ccccc32)c1] | D | LC-MS (ES⁺, Method A), 0.66 min, m/z 450.2 [M + H]⁺ | ¹H NMR (400 MHz, DMSO-d₆) δ 10.53 (s, 1H), 9.15 (s, 1H), 8.88 - 8.87 (m, 1H), 8.48 - 8.47 (m, 1H), 8.25 - 8.18 (m, 2H), 7.99 (s, 1H), 7.87 (d, J = 8.8 Hz, 1H), 7.62 - 7.46 (m, 7H), 7.24 (t, J = 7.2 Hz 1H), 6.64 (d, J = 1.6 Hz 1H), 3.89 (s, 2H) |
| 31 | 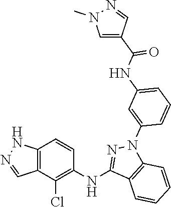 [SMILES: Cn1cc(C(=O)Nc2cccc(-n3nc(Nc4ccc5[nH]ncc5c4Cl)c4ccccc43)c2)cn1] | C | LC-MS (ES⁺, Method A), 0.50 min, m/z 483.2 [M + H]⁺ | ¹H NMR (400 MHz, CD-C, DMSO-d₆) & 10.08 (s, 1H), 8.39 (s, 1H), 8.19-8.15 (m, 1H), 8.07 (d, J = 6.4 Hz, 2H), 8.02 (d, J = 8.4 Hz, 1H), 7.90-7.87 (m, 2H), 7.64 (d, J = 8.4 Hz, 1H), 7.60-7.50 (m, 2H), 7.46-7.42 (m, 1H), 7.38- 7.36 (m, 1H), 7.20 (t, J = 7.6 Hz, 1H), 3.89 (s, 3H), exchangeable NH not seen. |
| 32 | 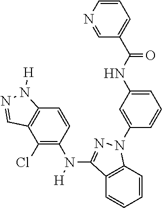 [SMILES: O=C(Nc1cccc(-n2nc(Nc3ccc4[nH]ncc4c3Cl)c3ccccc32)c1)c1cccnc1] | D | LC-MS (ES⁺, Method A), 0.47 min, m/z 480.1 [M + H]⁺ | ¹H NMR (400 MHz, DMSO-d₆) δ 10.63 (s, 1H), 9.14 (d, J = 2.0 Hz, 1H), 8.79 (dd, J = 4.8 Hz, 1.6 Hz, 1H), 8.46 (s, 1H), 8.37-8.35 (m, 1H), 8.19 (s, 1H), 8.08 (s, 1H), 8.02 (d, J = 8.0 Hz, 1H), 7.94-7.85 (m, 2H), 7.69 (d, J = 7.6 Hz, 1H), 7.65-7.58 (m, 1H), 7.57-7.40 (m, 4H), 7.21 (t, J = 7.2 Hz, 1H), exchangeable NH not seen. |

### General Route or the Synthesis of Examples 33-35:

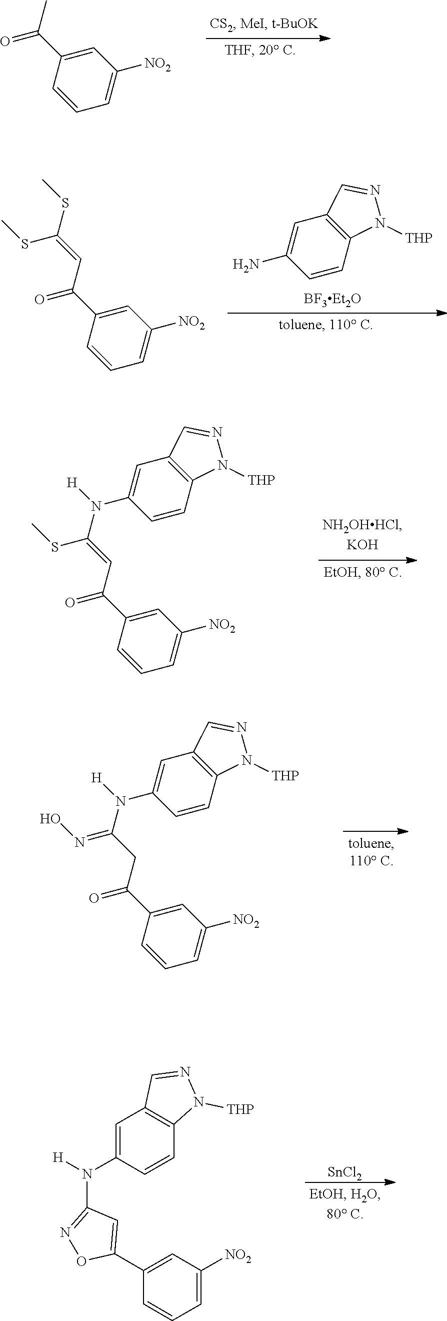

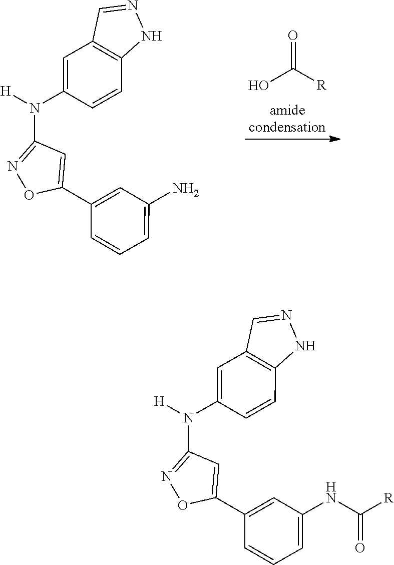

### Step 1: 3,3-bis(methylthio)-1-(3-nitrophenyl)prop-2-en-1-one

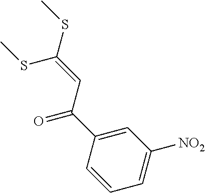 [SMILES: CSC(=CC(=O)c1cccc([N+](=O)[O-])c1)SC]

To the solution of 1-(3-nitrophenyl)ethanone (2 g, 12.11 mmol) and CS₂ (2.03 g, 26.64 mmol) in THF (20 mL), was added t-BuOK (1 M, 26.64 mL) at 0° C. under N₂. The mixture was stirred at r.t. for 0.5 hr. The CH₃I (8.59 g, 60.55 mmol) was added to the mixture and the mixture was stirred at r.t. for 0.5 hr. The reaction mixture was poured into water (100 mL), extracted with Ethyl acetate (100 mL×2). The combined organic layers were washed with brine (200 mL), dried over sodium sulfate, filtered and concentrated under reduced pressure to give 3,3-bis(methylthio)-1-(3-nitrophenyl)prop-2-en-1-one (3.0 g, 11.14 mmol, 92% yield) as a yellow solid. LC-MS (ES⁺, Method A), 0.64 min, m/z 270.0 [M+H]⁺.

### Step 2: (Z)-3-(methylthio)-1-(3-nitrophenyl)-3-((1-(tetrahydro-2H-pyran-2-yl)-1H-indazol-5-yl)amino)prop-2-en-1-one

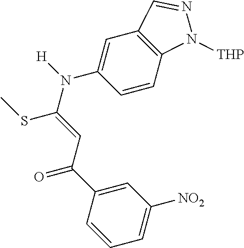

To the solution of 3,3-bis(methylthio)-1-(3-nitrophenyl)prop-2-en-1-one (2.40 g, 8.91 mmol) and 1-(tetrahydro-2H-pyran-2-yl)-1H-indazol-5-amine (2.90 g, 13.37 mmol) in toluene (72 mL) was added BF₃.Et₂O (126 mg, 891 μmol). The mixture was stirred at 110° C. for 16 hr. The reaction mixture was concentrated under reduced pressure to give a residue. The residue was poured into water (50 mL), extracted with ethyl acetate (100 mL×3). The combined organic layers were washed with brine (100 mL), dried over sodium sulfate, filtered and concentrated under reduced pressure to give a residue. The residue was purified by reversed-phase MPLC (FA conditions) to give (Z)-3-(methylthio)-1-(3-nitrophenyl)-3-((1-(tetrahydro-2H-pyran-2-yl)-1H-indazol-5-yl)amino)prop-2-en-1-one (1.9 g, 3.86 mmol, 43% yield) as a yellow oil. LC-MS (ES⁺, Method A), 0.74 min, m/z 439.2 [M+H]⁺.

### Step 3: (Z)—N′-hydroxy-3-(3-nitrophenyl)-3-oxo-N-(1-(tetrahydro-2H-pyran-2-yl)-1H-indazol-5-yl)propanimidamide

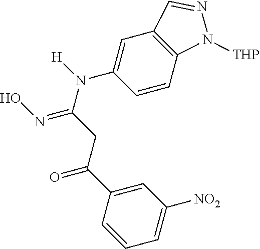

To the solution NH₂OH·HCl (950 mg, 13.68 mmol) and (Z)—3-(methylthio)-1-(3-nitrophenyl)-3-((1-(tetrahydro-2H-pyran-2-yl)-1H -indazol-5-yl)amino)prop-2-en-1-one (1.5 g, 3.42 mmol) in EtOH (200 mL) was added KOH (768 mg, 13.68 mmol), the mixture was stirred at 80° C. for 2 hr. The reaction mixture was concentrated under reduced pressure to give (Z)—N-hydroxy-3-(3-nitrophenyl)-3-oxo-N-(1-(tetrahydro-2H-pyran-2-yl)-1H-indazol-5-yl)propanimidamide (2 g, crude) as a yellow solid. LC-MS (ES⁺, Method B), 0.74 min, m/z 424.4 [M+H]⁺.

### Step 4: 5-(3-nitrophenyl)-N-(1-(tetrahydro-2H-pyran-2-yl)-1H-indazol-5-yl)isoxazol-3-amine

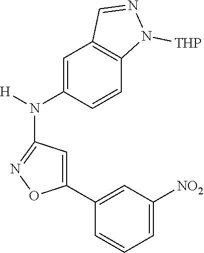

A solution of (Z)—N-hydroxy-3-(3-nitrophenyl)-3-oxo-N-(1-(tetrahydro-2H-pyran-2-yl)-1H-indazol-5-yl)propanimidamide (2 g, 4.72 mmol) in toluene (200 mL) was stirred at 110° C. for 3 hr. The reaction mixture was concentrated under reduced pressure to give the residue. The residue was purified by preparative HPLC (55-85% MeCN in H₂O) to give 5-(3-nitrophenyl)-N-(1-(tetrahydro-2H-pyran-2-yl)-1H-indazol-5-yl)isoxazol-3-amine (280 mg, 690.67 μmol, 56% yield) as a yellow solid. LC-MS (ES⁺, Method A), 0.70 min, m/z 406.1 [M+H]⁺.

### Step 5: 5-(3-aminophenyl)-N-(1H-indazol-5-yl)isoxazol-3-amine

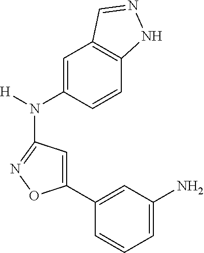 [SMILES: Nc1cccc(-c2cc(Nc3ccc4[nH]ncc4c3)no2)c1]

To a solution of 5-(3-nitrophenyl)-N-(1-(tetrahydro-2H-pyran-2-yl)-1H-indazol-5-yl)isoxazol-3-amine (250 mg, 616.67 μmol) in EtOH (10 mL) and H₂O (0.5 mL) was added SnCl₂2H₂O (695.7 mg, 3.08 mmol) in N one portion at r.t., and the reaction solution was stirred at 80° C. for 2 hr. The reaction mixture was concentrated under reduced pressure to give the residue. The residue was purified by preparative HPLC (12-42% MeCN in H₂O) to give 5-(3-aminophenyl)-N-(1H-indazol-5-yl)isoxazol-3-amine (70.1 mg, 226.33 μmol, 37% yield) as a yellow solid. LC-MS (ES⁺, Method A), 0.45 min, m/z 292.1 [M+H]⁺.

General Method G for the synthesis of example 34:

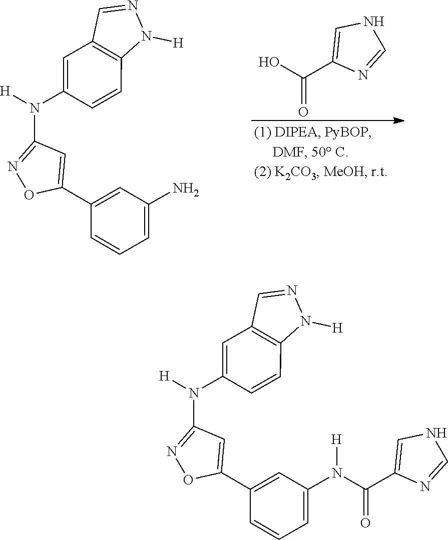 [SMILES: Nc1cccc(-c2cc(Nc3ccc4[nH]ncc4c3)no2)c1 ; O=C(O)c1c[nH]cn1 ; O=C(Nc1cccc(-c2cc(Nc3ccc4[nH]ncc4c3)no2)c1)c1c[nH]cn1]

### Example 34: N-(3-(3-((1H-indazol-5-yl)amino)isoxazol-5-yl)phenyl)-1H-imidazole-4-carboxamide

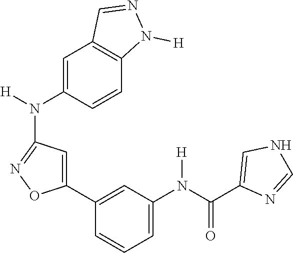 [SMILES: O=C(Nc1cccc(-c2cc(Nc3ccc4[nH]ncc4c3)no2)c1)c1c[nH]cn1]

To a solution of 5-(3-aminophenyl)-N-(1H-indazol-5-yl)isoxazol-3-amine (51 mg, 175.07 μmol) and 1H-imidazole-4-carboxylic acid (39 mg, 350.15 μmol) in DMF (1 mL) was added DIPEA (68 mg, 525.22 μmol) and PyBOP (182 mg, 350.15 μmol) at r.t. Then the mixture was stirred at 50° C. for 48 hr. The reaction mixture was poured into water (20 mL) and filtered. The filter cake was washed by EtOAc (30 mL). Then the mixture of filter cake in MeOH (2 mL) was added K₂CO₃ (60 mg) and the mixture stirred at r.t. for 0.5 h. The mixture was diluted with water (20 mL) and filtered. The filter cake was washed with EtOAc (30 mL). The crude product was purified by preparative HPLC (12-42% MeCN in H₂O) to give N-(3-(3-((1H-indazol-5-yl)amino)isoxazol-5-yl)phenyl)-1H-imidazole-4-carboxamide (20.90 mg, 53.69 μmol, 31% yield) as an off-white solid. LC-MS (ES⁺, Method A), 0.37 min, m/z 386.2 [M+H]⁺. ¹H NMR (400 MHz, DMSO-d₆) δ 12.89 (s, 1H), 12.68 (s, 1H), 10.04 (s, 1H), 9.19 (s, 1H), 8.36 (s, 1H), 7.99-7.97 (m, 3H), 7.90-7.80 (m, 2H), 7.55-7.53 (m, 1H), 7.50-7.45 (m, 2H), 7.32 (d, J=9.2 Hz, 1H), 6.52 (s, 1H).

Compounds prepared in a similar manner to that set out above are given below in Table 8:

TABLE 8

| Example | Structure | Method | LCMS | ¹H NMR |
|---|---|---|---|---|
| 33 | 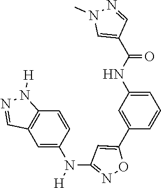 [SMILES: Cn1cc(C(=O)Nc2cccc(-c3cc(Nc4ccc5[nH]ncc5c4)no3)c2)cn1] | C | LC-MS (ES⁺, Method A), 0.55 min, m/z 400.1 [M + H]⁺ | ¹H NMR (400 MHz, DMSO-d₆) δ 3.91 (s, 2H), 3.89-3.89 (m, 1H), 6.53 (s, 1H), 7.32-7.33 (m, 1H), 7.47-7.52 (m, 2H), 7.54-7.62 (m, 1H), 7.88 (dd, J = 7.2, 1.2 Hz, 1H), 7.96-8.01 (m, 1H), 7.97-8.00 (m, 1H), 8.02-8.08 (m, 1H), 8.05 (s, 1H), 8.18 (t, J = 1.6 Hz, 1H), 8.35 (s, 1H), 9.19 (s, 1H), 10.01 (s, 1H), 12.90 (s, 1H). |
| 35 | 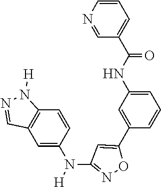 [SMILES: O=C(Nc1cccc(-c2cc(Nc3ccc4[nH]ncc4c3)no2)c1)c1cccnc1] | C | LC-MS (ES⁺, Method A), 0.43 min, m/z 397.1 [M + H]⁺ | ¹H NMR (400 MHz, DMSO-d₆) δ 12.88 (s, 1H), 10.64 (s, 1H), 9.18-9.15 (m, 2H), 8.80-8.79 (m, 1H), 8.36-8.33 (m, 1H), 8.27 (s, 1H), 8.00-7.95 (s, 2H), 7.93 (d, J = 8.4 Hz, 1H), 7.64-7.59 (m, 2H), 7.57-7.49 (m, 2H), 7.40-7.20 (m, 1H), 6.56 (s, 1H) |

### General Route or the Synthesis of Examples 36-38:

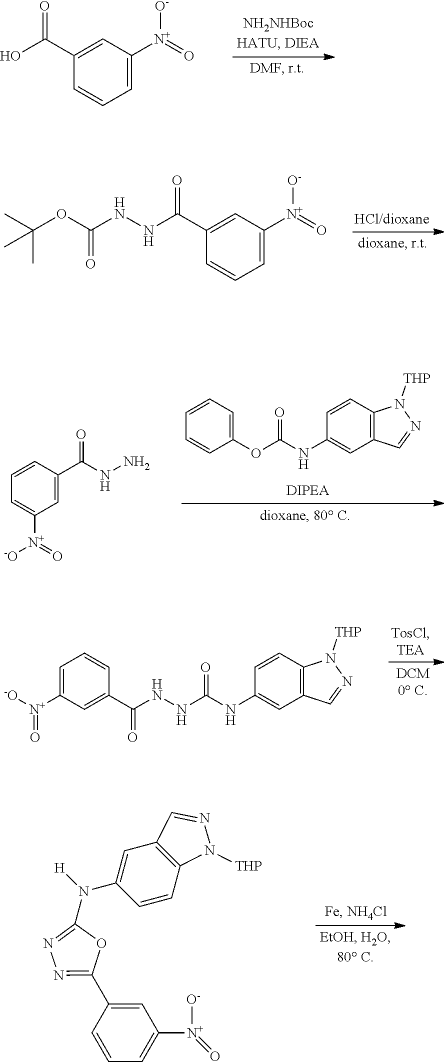

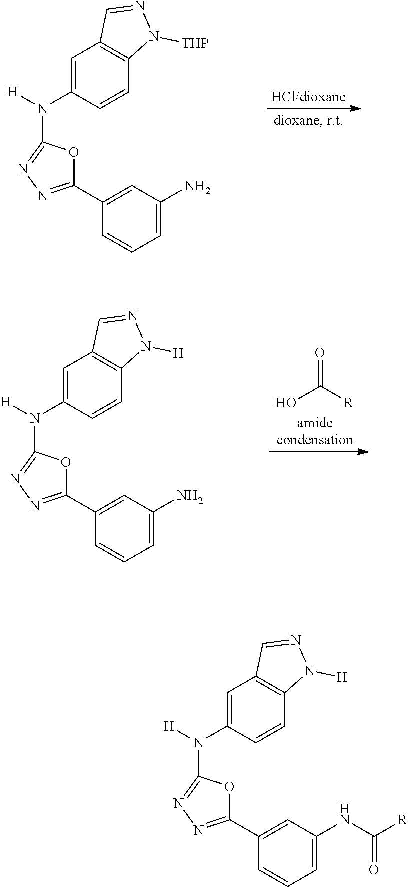

### Step 1: tert-butyl 2-(3-nitrobenzoyl)hydrazinecarboxylate

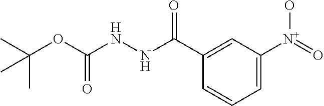

To a solution of 3-nitrobenzoic acid (5 g, 29.92 mmol) and tert-butyl hydrazinecarboxylate (3.95 g, 29.92 mmol) in DMF (250 mL) was added HATU (11.38 g, 29.92 mmol) and DIEA (5.80 g, 44.88 mmol) at r.t. The reaction was stirred at r.t. for 12 hr. The reaction was partitioned between H₂O (1000 mL) and EtOAc (150 mL). The organic layer was separated and the aqueous layer was extracted with EtOAc (3×150 mL). The combined organics were washed with brine (2×100 mL), dried over sodium sulfate, filtered and the solvent removed under reduced pressure. The residue was loaded onto silica and purified by column chromatography eluting with 0-75% EtOAc in Pet. Ether to give tert-butyl 2-(3-nitrobenzoyl)hydrazinecarboxylate (8.20 g, 29.15 mmol, 97% yield) as a white solid. LC-MS (ES⁺, Method A), 0.47 min, m/z 225.9 [M-56+H]⁺.

### Step 2: 3-nitrobenzohydrazide

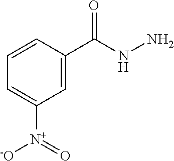

To a solution of tert-butyl 2-(3-nitrobenzoyl)hydrazinecarboxylate (7 g, 24.89 mmol) in dioxane (50 mL) was added HCl/dioxane (4M, 50 mL) at r.t and the solution was stirred at r.t. for 12 hr. The reaction mixture was filtered and the filter cake was added into NaHCO₃ aqueous solution (1 M), the solution was stirred at r.t. for 2 h. The reaction was partitioned between H₂O (50 mL) and EtOAc (50 mL). The organic layer was separated and the aqueous layer was extracted with EtOAc (3×50 mL). The combined organics were washed with brine (2×20 mL), dried over sodium sulfate, filtered and the solvent removed under reduced pressure to give 3-nitrobenzohydrazide (3.50 g, 19.32 mmol, 78% yield) as a white solid. LC-MS (ES⁺, Method A), 0.18 min, m/z 182.2 [M+H]⁺.

### Step 3: 2-(3-nitrobenzoyl)-N-(1-(tetrahydro-2H-pyran-2-yl)-1H-indazol-5-yl)hydrazinecarboxamide

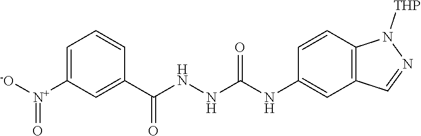

To a solution of phenyl (1-(tetrahydro-2H-pyran-2-yl)-1H-indazol-5-yl)carbamate (1.25 g, 3.71 mmol) and 3-nitrobenzohydrazide (671 mg, 3.71 mmol) in dioxane (30 mL) was added DIEA (958 mg, 7.41 mmol) at r.t. the reaction was stirred at 80° C. for 12 hr. The reaction mixture was diluted with H₂O (50 mL) and acidified to pH=7 by 1 M HCl aqueous solution. The mixture was filtered and the filter cake was dried to give 2-(3-nitrobenzoyl)-N-(1-(tetrahydro-2H-pyran-2-yl)-1H-indazol-5-yl)hydrazinecarboxamide (1.37 g, 3.23 mmol, 87% yield) as a light yellow solid. LC-MS (ES⁺, Method A), 0.50 min, m/z 425.3 [M+H]⁺.

### Step 4: 5-(3-nitrophenyl)-N-(1-(tetrahydro-2H-pyran-2-yl)-1H-indazol-5-yl)-1,3,4-N oxadiazol-2-amine

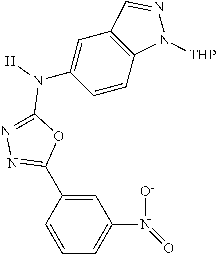

To a solution of 2-(3-nitrobenzoyl)-N-(1-(tetrahydro-2H-, pyran-2-yl)-1H-indazol-5-yl)hydrazinecarboxamide (1.36 g, 3.20 mmol) in DCM (50 mL) was added 4-methylbenzenesulfonyl chloride (855 mg, 4.49 mmol) and TEA (973 mg, 9.61 mmol) at 0° C., and the reaction was stirred at 0° C. for 1.5 hr. The reaction mixture was filtered and the filter

cake was dried to give 5-(3-nitrophenyl)-N-(1-(tetrahydro-2H-pyran-2-yl)-1H-indazol-5-yl)-1,3,4-oxadiazol-2-amine (660 mg, 1.62 mmol, 51% yield) as a light yellow solid. LC-MS (ES⁺, Method A), 0.60 min, m/z 407.2 [M+H]⁺.

### Step 5: 5-(3-aminophenyl)-N-(1-(tetrahydro-2H-pyran-2-yl)-1H-indazol-5-yl)-1,3,4-oxadiazol-2-amine

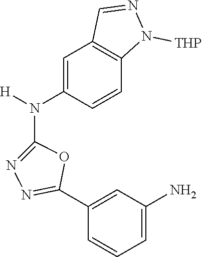

To a solution of 5-(3-nitrophenyl)-N-(1-(tetrahydro-2H-pyran-N 2-yl)-1H-indazol-5-yl)-1,3,4-oxadiazol-2-amine (650 mg, 1.60 mmol) in EtOH (12 mL) and H₂O (6 mL) was added Fe (447 mg, 8.00 mmol) and NH₄Cl (856 mg, 15.99 mmol) at r.t. The reaction was stirred at 80° C. for 1 hr. It was then cooled to r.t. Then the reaction mixture was diluted with EtOAc (50 mL). The solution was filtered and filter cake was triturated with EtOAc (10 mL) to give 5-(3-aminophenyl)-N-(1-(tetrahydro-2H-pyran-2-yl)-1H-indazol-5-yl)-1,3,4-oxadiazol-2-amine (490 mg, 1.30 mmol, 81% yield) as a light yellow solid. LC-MS (ES⁺, Method A), 0.44 min, m/z 377.3 [M+H]⁺.

### Step 6: 5-(3-aminophenyl)-N-(1H-indazol-5-yl)-1,3,4-oxadiazol-2-amine

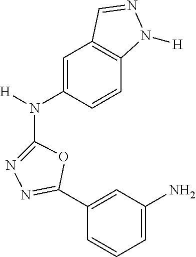 [SMILES: Nc1cccc(-c2nnc(Nc3ccc4[nH]ncc4c3)o2)c1]

To a solution of 5-(3-aminophenyl)-N-(1-(tetrahydro-2H-pyran-2-yl)-1H-indazol-5-yl)-1,3,4-oxadiazol-2-amine (430 mg, 1.14 mmol) in dioxane (10 mL) was added HCl/dioxane (4M, 10 mL) at r.t. The reaction was stirred at r.t. for 2.5 hr. The reaction mixture was filtered and the filter cake was dried to give 5-(3-aminophenyl)-N-(1H-indazol-5-yl)-1,3,4-oxadiazol-2-amine (270 mg, 821.28 μmol, 72% yield) as a light yellow solid. LC-MS (ES⁺, Method A), 0.35 min, m/z 393.3 [M+H]⁺.

Compounds prepared in a similar manner to that set out above (methods as examples 1 to 5) are given below in Table 9:

| Example | Structure | Method | LCMS | ¹H NMR |
|---|---|---|---|---|
| 36 | 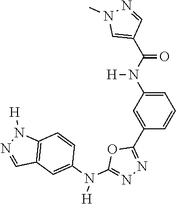 [SMILES: Cn1cc(C(=O)Nc2cccc(-c3nnc(Nc4ccc5[nH]ncc5c4)o3)c2)cn1] | D | LC-MS (ES⁺, Method A), 0.45 min, m/z 401.3 [M + H]⁺ | ¹H NMR (400 MHz, DMSO-d₆) δ 12.99 (s, 1H), 10.65 (s, 1H), 10.06 (s, 1H), 8.46 (s, 1H), 8.35 (s, 1H), 8.16 (d, J = 1.4 Hz, 1H), 8.06 (s, 2H), 7.86 (d, J = 8.0 Hz, 1H), 7.61-7.46 (m, 4H), 3.91 (s, 3H) |
| 37 | 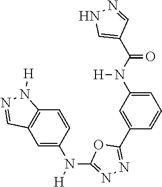 [SMILES: O=C(Nc1cccc(-c2nnc(Nc3ccc4[nH]ncc4c3)o2)c1)c1cn[nH]c1] | E | LC-MS (ES⁺, Method A), 0.35 min, m/z 387.1 [M + H]⁺ | ¹H NMR (400 MHz, DMSO-d₆) δ 12.98 (s, 1H), 10.66 (s, 1H), 10.46 (s, 1H), 8.53 (s, 2H), 8.16-8.12 (m, 2H), 8.06 (s, 1H), 7.91-7.87 (m, 1H), 7.66-7.61 (m, 1H), 7.59-7.53 (m, 2H), 7.50-7.46 (m, 1H), exchangeable NH not seen |
| 38 | 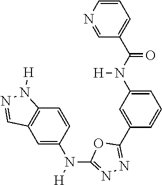 [SMILES: O=C(Nc1cccc(-c2nnc(Nc3ccc4[nH]ncc4c3)o2)c1)c1cccnc1] | D | LC-MS (ES⁺, Method A), 0.38 min, m/z 398.1 [M + H]⁺ | ¹H NMR (400 MHz, DMSO-d₆) δ 13.01 (s, 1H), 10.71 (s, 1H), 10.66 (s, 1H), 9.17 (d, J = 1.2 Hz, 1H), 8.81 (d, J = 4.0 Hz, 1H), 8.55 (s, 1H), 8.38 (d, J = 8.0 Hz, 1H), 8.16 (s, 1H), 8.07 (s, 1H), 7.89-7.86 (m, 1H), 7.69-7.54 (m, 4H), 7.50-7.47 (m, 1H) |

### General Route or the Synthesis of Examples 39-41:

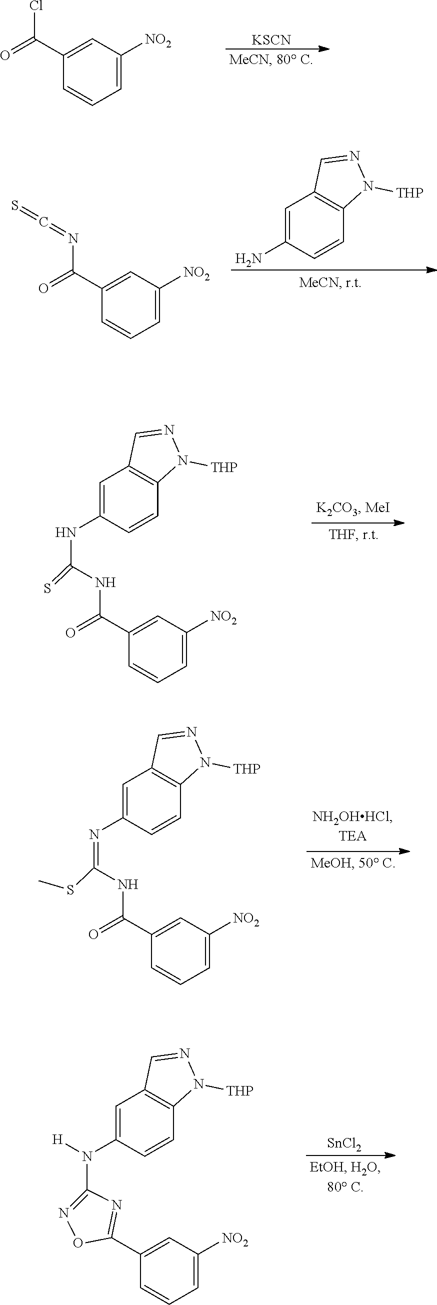

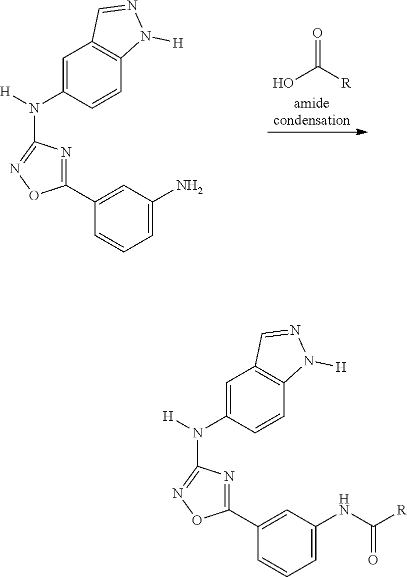

### Step 1: 3-nitrobenzoyl isothiocyanate

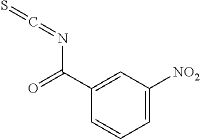 [SMILES: O=C(N=C=S)c1cccc([N+](=O)[O-])c1]

A solution of 3-nitrobenzoyl chloride (3.5 g, 18.86 mmol) and KSCN (1.83 g, 18.86 mmol) in MeCN (40 mL) was stirred at 80° C. for 1 hr. The mixture was concentrated under reduced pressure to give 3-nitrobenzoyl isothiocyanate (3.93 g, crude) as a yellow solid.

### Step 2: 3-nitro-N-((1-(tetrahydro-2H-pyran-2-yl)-1H-indazol-5-yl)carbamothioyl)benzamide

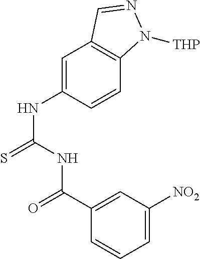

A solution of 3-nitrobenzoyl isothiocyanate (3.93 g, 18.88 mmol) 1-(tetrahydro-2H-pyran-2-yl)-1H-indazol-5-amine (4.10 g, 18.88 mmol) in MeCN (40 mL) was stirred at r.t. for 1 hr. The reaction solution was filtered and the filter cake was washed by MeCN (20 mL) and water (10 mL). The filter cake was collected to give 3-nitro-N-((1-(tetrahydro-2H-pyran-2-yl)-1H-indazol-5-yl)carbamothioyl)benzamide (7.15 g, 16.81 mmol, 89% yield) as a yellow solid. LC-MS (ES⁺, Method A), 0.65 min, m/z 426.3 [M+H]+*.

### Step 3: (E)-methyl N-(3-nitrobenzoyl)-N′-(1-(tetrahydro-2H-pyran-2-yl)-1H-indazol-5-yl)carbamimidothioate

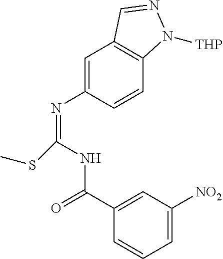

To a solution of 3-nitro-N-((1-(tetrahydro-2H-pyran-2-yl)-1H-indazol-5-yl)carbamothioyl)benzamide (5 g, 11.75 mmol) and CH₃I (2.50 g, 17.63 mmol) in THE (75 mL) was added K₂CO₃ (3.25 g, 23.50 mmol), the reaction solution was stirred at r.t. for 1.5 hr. The reaction solution was filtered and the filter cake was washed by water (40 mL). The filter cake was collected to give (E)-methyl N-(3-nitrobenzoyl)-N-(1-(tetrahydro-2H-pyran-2-yl)-1H-indazol-5-yl) carbamimidothioate (2.82 g, 6.42 mmol, 55% yield) as a white solid. LC-MS (ES⁺, Method A), 0.71 min, m/z 440.1 [M+H]⁺.

### Step 4: 5-(3-nitrophenyl)-N-(1-(tetrahydro-2H-pyran-2-yl)-1H-indazol-5-yl)-1,2,4-oxadiazol-3-amine

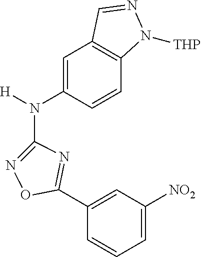

To a solution of (E)-methyl N-(3-nitrobenzoyl)-N-(1-(tetrahydro-2H-pyran-2-yl)-1H-indazol-5-yl)carbamimidothioate (2.82 g, 6.42 mmol) in MeOH (60 mL) was added NH₂OH·HCl (1.34 g, 19.2 NN mmol) and TEA (3.90 g, 38.5 mmol), and the reaction solution was stirred b NO₂ at 50° C. for 66 hr. The reaction solution was filtered and the filter cake was washed by MeOH (20 mL). The filter cake was collected to give 5-(3-nitrophenyl)-N-(1-(tetrahydro-2H-pyran-2-yl)-1H-indazol-5-yl)-1,2,4-oxadiazol-3-amine (1.5 g, 3.69 mmol, 58% yield) as a yellow solid. LC-MS (ES⁺, Method A), 0.63 min, m/z 407.1 [M+H]⁺.

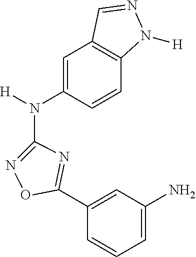 [SMILES: Nc1cccc(-c2nc(Nc3ccc4[nH]ncc4c3)no2)c1]

### Step 5: 5-(3-aminophenyl)-N-(1H-indazol-5-yl)-1,2,4-oxadiazol-3-amine

To a solution of 5-(3-nitrophenyl)-N-(1-(tetrahydro-2H-pyran-2-˜H yl)-1H-indazol-5-yl)-1,2,4-oxadiazol-3-amine (200 mg, 492.13 μmol) in EtOH (5 mL) and H₂O (0.2 mL) was added SnCl₂.2H₂O (555 mg, 2.46 mmol), and the reaction solution was stirred at 80° C. for 2 hr. The reaction solution was concentrated under reduced pressure, the residue was added ethyl acetate (30 mL) and sodium bicarbonate solution (10%, 10 mL). The solution was filtered and the filtrate was concentrated under reduced pressure. The residue was purified by preparative HPLC (19-49% MeCN in H₂O) to give 5 5-(3-aminophenyl)-N-(1H-indazol-5-yl)-1,2,4-oxadiazol-3-amine (110 mg, 210.13 μmol, 43% yield) as a white solid. LC-MS (ES⁺, Method A), 0.49 min, m/z 293.2 [M+H]⁺.

Compounds prepared in a similar manner to that set out above are given below in Table 10:

| Example | Structure | Method | LCMS | ¹H NMR |
|---|---|---|---|---|
| 39 | 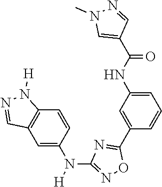 [SMILES: Cn1cc(C(=O)Nc2cccc(-c3nc(Nc4ccc5[nH]ncc5c4)no3)c2)cn1] | D | LC-MS (ES⁺, Method A), 0.57 min, m/z 401.1 [M + H]⁺ | ¹H NMR (400 MHz, DMSO-d₆) δ 13.18-12.75 (m, 1H), 10.13 (s, 1H), 10.0-9.96 (m, 1H), 8.63-8.58 (m, 1H), 8.39-8.34 (m, 1H), 8.09-8.05 (m, 1H), 8.05-8.03 (m, 1H), 8.01-7.97 (m, 1H), 7.97-7.94 (m, 1H), 7.80- 7.75 (m, 1H), 7.64-7.57 (m, 1H), 7.55-7.50 (m, 1H), 7.47-7.41 (m, 1H), 3.91 (s, 3H) |
| 40 | 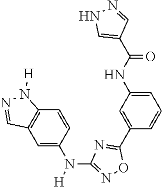 [SMILES: O=C(Nc1cccc(-c2nc(Nc3ccc4[nH]ncc4c3)no2)c1)c1cn[nH]c1] | E | LC-MS (ES⁺, Method A), 0.48 min, m/z 387.1 [M + H]⁺ | ¹H NMR (400 MHz, DMSO-d₆) δ 13.13 − 12.76 (m, 1H), 10.47 (s, 1H), 10.01 (s, 1H), 8.70 (s, 1H), 8.38 (s, 1H), 8.09 - 8.06 (m, 1H), 8.05-8.01 (m, 2H), 7.9 -7.95 (m, 1H), 7.83-7.79 (m, 1H), 7.65-7.59 (m, 1H), 7.54- 7.50 (m, 1H), 7.46-7.41 (m, 1H) |
| 41 | 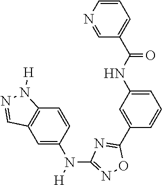 [SMILES: O=C(Nc1cccc(-c2nc(Nc3ccc4[nH]ncc4c3)no2)c1)c1cccnc1] | D | LC-MS (ES⁺, Method A), 0.52 min, m/z 398.1 [M + H]⁺ | ¹H NMR (400 MHz, DMSO-d₆) δ 13.13-12.80 (m, 1H), 10.77 (s, 1H), 10.02 (s, 1H), 9.16 (d, J = 1.6 Hz, 1H), 8.84-8.67 (m, 2H), 8.40-8.29 (m, 1H), 8.08-8.00 (m, 2H), 7.99-7.94 (m, 1H), 7.87-7.81 (m, 1H), 7.69- 7.59 (m, 2H), 7.56-7.49 (m, 1H), 7.47-7.41 (m, 1H) |

### General Route or the Synthesis of Examples 42-44:

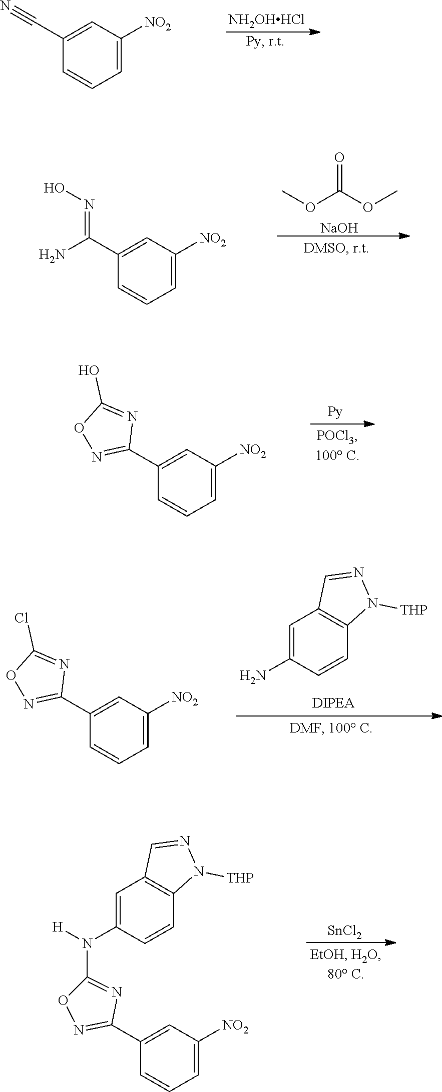

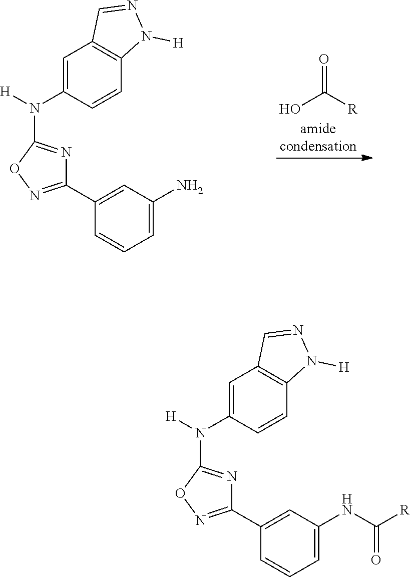

### Step 1: (Z)—N′-hydroxy-3-nitrobenzimidamide

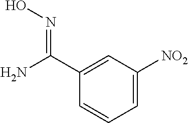 [SMILES: N/C(=N\O)c1cccc([N+](=O)[O-])c1]

To a solution of hydroxylamine; hydrochloride (14.1 g, 202.6 mmol) in pyridine (100 mL) was added 3-nitrobenzonitrile (5 g, 33.76 mmol) at 0° C., and the reaction solution was stirred at r.t. for 16 hr. The reaction mixture was poured into water (100 mL), extracted with ethyl acetate (100 mL×3). The combined organic layers were washed with brine (100 mL×2), dried over sodium sulfate, filtered and concentrated under reduced pressure to give (Z)—N′-hydroxy-3-nitrobenzimidamide (6 g, 33.12 mmol, 98 yield) as a yellow solid.

### Step 2: 3-(3-nitrophenyl)-1,2,4-oxadiazol-5-ol

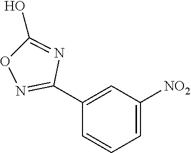 [SMILES: O=[N+]([O-])c1cccc(-c2noc(O)n2)c1]

To the solution of (Z)—N′-hydroxy-3-nitrobenzimidamide (3.5 g, 19.32 mmol) and dimethyl carbonate (2.61 g, 28.98 mmol) in DMSO (10 mL) was added NaOH (1.16 g, 28.98 mmol). The reaction solution was stirred at r.t. for 16 hr. The reaction was diluted with H₂O (10 mL), and con. HCl (15 mL) was added to the solution, then filtered and washed with H₂O (30 mL). The filter cake was collected to give 3-(3-nitrophenyl)-1,2,4-oxadiazol-5-ol (2.5 g, 12.07 mmol, 62% yield) as a yellow solid.

### Step 3: 5-chloro-3-(3-nitrophenyl)-1,2,4-oxadiazole

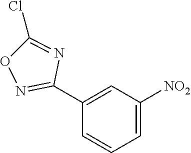 [SMILES: O=[N+]([O-])c1cccc(-c2noc(Cl)n2)c1]

To a stirred mixture of 3-(3-nitrophenyl)-1,2,4-oxadiazol-5-ol (1 g, 4.83 mmol) in POCl₃ (20 mL) was added Py (572.8 mg, 7.24 mmol). The mixture was heated at 100° C. for 16 hr. The reaction mixture was concentrated under reduced pressure to remove solvent. The residue was added dropwise to ice water (100 mL) and extracted with ethyl acetate (30 mL×3). The combined organic layers were washed with brine (50 mL×2), dried over sodium sulfate, filtered and concentrated under reduced pressure to give 5-chloro-3-(3-nitrophenyl)-1,2,4-oxadiazole (940 mg, 4.17 mmol, 86% yield) as a white solid.

### Step 4: 3-(3-nitrophenyl)-N-(1-(tetrahydro-2H-pyran-2-yl)-1H-indazol-5-yl)-1,2,4-oxadiazol-5-amine

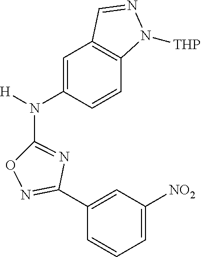

To a solution of 1-(tetrahydro-2H-pyran-2-yl)-1H-indazol-5-amine (443.03 mg, 2.04 mmol) in DMF (15 mL) was added DIPEA (790.62 mg, 6.12 mmol), and then 5-chloro-3-(3-nitrophenyl)-1,2,4-oxadiazole (460 mg, 2.04 mmol) was added to the mixture. The reaction was stirred at 100° C. for 16 hr. The reaction mixture was poured into water (100 mL), extracted with ethyl acetate (100 mL×2). The combined organic layers were washed with brine (200 mL), dried over sodium sulfate, filtered and concentrated under reduced pressure to give a residue. The residue was purified by column chromatography eluting with 10-50% EtOAc in Pet. Ether to give 3-(3-nitrophenyl)-N-(1-(tetrahydro-2H-pyran-2-yl)-1H-indazol-5-yl)-1,2,4-oxadiazol-5-amine (1.4 g, 3.44 mmol, 84% yield) as a red oil. LC-MS (ES⁺, Method A), 0.66 min, m/z 407.3 [M+H]⁺.

### Step 5: 3-(3-aminophenyl)-N-(1H-indazol-5-yl)-1,2,4-oxadiazol-5-amine

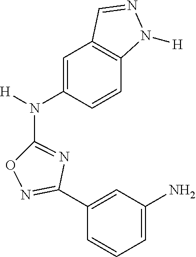 [SMILES: Nc1cccc(-c2noc(Nc3ccc4[nH]ncc4c3)n2)c1]

To a solution of 3-(3-nitrophenyl)-N-(1-(tetrahydro-2H-pyran-2-yl)-1H-indazol-5-yl)-1,2,4-oxadiazol-5-amine (400 mg, 984.27 μmol) in EtOH (12 mL) and H₂O (0.4 mL) was added SnCl₂.2H₂O (1.11 g, 4.92 mmol), and the reaction solution was stirred at 80° C. for 2 hr. The reaction was concentrated under reduced pressure to remove solvent. The residue was diluted with water (30 mL) and extracted with Ethyl acetate (30 mL×3). The combined organic layers were washed with brine (50 mL×2), dried over Na₂SO₄, filtered and concentrated under reduced pressure to give a residue. The residue was purified by preparative HPLC (8-38% MeCN in H₂O) to give 3-(3-aminophenyl)-N-(1H-indazol-5-yl)-1,2,4-oxadiazol-5-amine (180 mg, 597.34 μmol, 30% yield) as a white solid. LC-MS (ES⁺, Method A), 0.42 min, m/z 293.1 [M+H]⁺.

Compounds prepared in a similar manner to that set out above are given below in Table 11:

| Example | Structure | Method | LCMS | ¹H NMR |
|---|---|---|---|---|
| 42 | 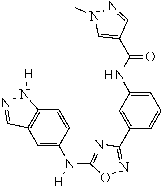 [SMILES: Cn1cc(C(=O)Nc2cccc(-c3noc(Nc4ccc5[nH]ncc5c4)n3)c2)cn1] | D | LC-MS (ES⁺, Method A), 0.55 min, m/z 401.1 [M + H]⁺ | ¹H NMR (400 MHz, DMSO-d₆) δ 13.08 (s, 1H), 11.00 (s, 1H), 10.04 (s, 1H), 8.35 (s, 2H), 8.13-8.08 (m, 2H), 8.06-8.04 (m, 1H), 8.02-7.98 (m, 1H), 7.73- 7.69 (m, 1H), 7.63-7.57 (m, 1H), 7.55-7.48 (m, 2H), 3.94 (s, 3H) |
| 43 | 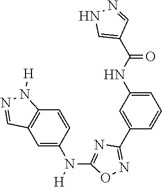 [SMILES: O=C(Nc1cccc(-c2noc(Nc3ccc4[nH]ncc4c3)n2)c1)c1cn[nH]c1] | E | LC-MS (ES⁺, Method A), 0.39 min, m/z 387.0 [M + H]⁺ | ¹H NMR (400 MHz, CD₃OD-d₄) δ 8.76 (s, 1H), 8.42 (s, 1H), 8.26-8.22 (m, 1H), 8.18 (s, 1H), 8.07 (s, 1H), 7.96 (dd, J = 1.2, 8.0 Hz, 1H), 7.90-7.85 (m, 1H), 7.61-7.50 (m, 3H) |
| 44 | 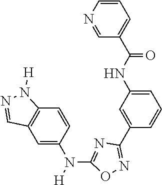 [SMILES: O=C(Nc1cccc(-c2noc(Nc3ccc4[nH]ncc4c3)n2)c1)c1cccnc1] | D | LC-MS (ES⁺, Method A), 0.43 min, m/z 398.2.0 [M + H]⁺ | ¹H NMR (400 MHz, DMSO-d₆) δ 13.27-12.89 (m, 1H), 11.02 (s, 1H), 10.68 (s, 1H), 9.16 (d, J = 2.4 Hz, 1H), 8.79 (dd, J = 1.6, 4.8 Hz, 1H), 8.46 (s, 1H), 8.36 (d, J = 8.0 Hz, 1H), 8.15-8.07 (m, 2H), 8.05-7.99 (m, 1H), 7.77 (d, J = 8.0 Hz, 1H), 7.64-7.49 (m, 4H), 2.07 (s, 1H) |

### General Route or the Synthesis of Examples 45-47:

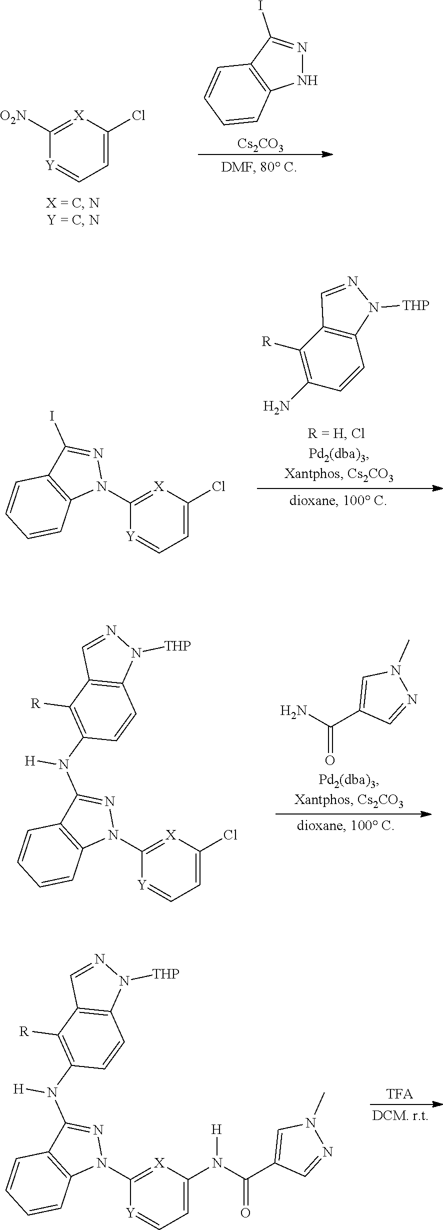

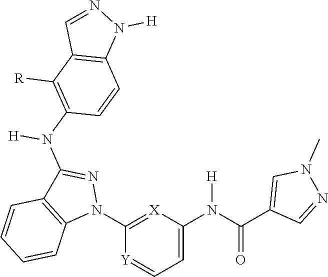

### Step 1: 1-(6-chloropyridin-2-yl)-3-iodo-1H-indazole

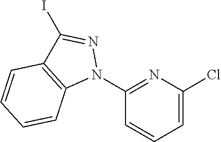 [SMILES: Clc1cccc(-n2nc(I)c3ccccc32)n1]

A mixture of 3-iodo-1H-indazole (0.2 g, 819.56 μmol), 2-N chloro-6-nitro-pyridine (130 mg, 819.56 μmol,) and Cs₂CO₃ (534 mg, 1.64 mmol) in DMF (5 mL) was degassed and purged with N₂ for 3 times, and then the mixture was stirred at 80° C. for 16 h under N₂ atmosphere. The reaction mixture was partitioned between EtOAc (50 mL) and water (20 mL). The organic phase was separated, washed with brine (20 mL×3), dried over Na₂SO₄, filtered and concentrated under reduced pressure to give a residue. The residue was purified by column chromatography eluting with 20-50% EtOAc in Pet. Ether to give 1-(6-chloropyridin-2-yl)-3-iodo-1H-indazole (0.25 g, 611.71 μmol, 75% yield) as a white solid. LC-MS (ES⁺, Method A), 0.87 min, m/z 355.8 [M+H]⁺.

### Step 2: 1-(6-chloropyridin-2-yl)-N-(1-(tetrahydro-2H-pyran-2-yl)-1H-indazol-5-yl)-1H-indazol-3-amine

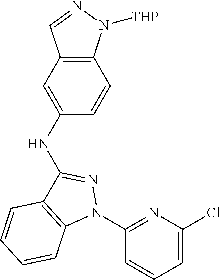

To a solution of 1-(6-chloropyridin-2-yl)-3-iodo-1H-indazole (240 mg, 674.99 [tmol) and 1-(tetrahydro-2H-pyran-2-yl)-1H-indazol-5-amine (147 mg, 674.99 μmol) in dioxane (6 mL) was added Xantphos (78 mg, 135.00 μmol) and Pd₂(dba)₃ (62 mg, 67.50 μmol) and Cs₂CO₃ (440 mg, 1.35 mmol) at r.t. The reaction was evacuated, flushed with nitrogen and stirred at 100° C. for 2 hr. The reaction was cooled to r.t. and solvent was removed under reduced pressure. The residue was partitioned between H₂O (10 mL) and EtOAc (10 mL). The organic layer was separated and the aqueous was extracted with EtOAc (3×10 mL). The combined organics were washed with brine (2×10 mL), dried over sodium sulfate, filtered and the solvent removed under reduced pressure. The residue was loaded onto silica and purified by column chromatography eluting with 0-33% EtOAc in Pet. Ether to give 1-(6-chloropyridin-2-yl)-N-(1-(tetrahydro-2H-pyran-2-yl)-1H-indazol-5-yl)-1H-indazol-3-amine (220 mg, 494.48 μmol, 73% yield) as a yellow solid. LC-MS (ES⁺, Method A), 0.80 min, m/z 445.3 [M+H]⁺.

### Step 3: 1-methyl-N-(6-(3-((1-(tetrahydro-2H-pyran-2-yl)-1H-indazol-5-yl)amino)-1H-indazol-1-yl)pyridin-2-yl)-1H-pyrazole-4-carboxamide

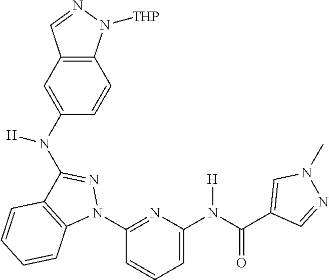

To a solution of give 1-(6-chloropyridin-2-yl)-N-(1-(tetrahydro-2H-pyran-2-yl)-1H-indazol-5-yl)-1H-indazol-3-amine (200 mg, 449.52 μmol) and 1-methyl-1H-pyrazole-4-carboxamide (84 mg, 674.29 μmol) in dioxane (4 mL) was added Pd₂(dba)₃ (41 mg, 44.95 μmol), Xantphos (52 mg, 89.90 μmol) and Cs₂CO₃ (293 mg, 899.05 μmol) at r.t. The reaction was evacuated, flushed with nitrogen and stirred at 100° C. for 5 hr. The reaction was cooled to r.t. and solvent removed under reduced pressure. The residue was partitioned between H₂O (10 mL) and EtOAc (10 mL). The organic layer was separated and the aqueous layer was extracted with EtOAc (3×10 mL). The combined organics were washed with brine (2×10 mL), dried over sodium sulfate, filtered and the solvent removed under reduced pressure. The residue was loaded onto silica and purified by column chromatography eluting with 0-100% EtOAc in Pet. Ether to give 1-methyl-N-(6-(3-((1-(tetrahydro-2H-pyran-2-yl)-1H-indazol-5-yl)amino)-1H-indazol-1-yl)pyridin-2-yl)-1H-pyrazole-4-carboxamide (120 mg, 224.89 μmol, 50% yield) as a yellow solid. LC-MS (ES⁺, Method A), 0.65 min, m/z 534.2 [M+H]⁺.

Example 45: N-(6-(3-((1H-indazol-5-yl)amino)-1H-indazol-1-yl)pyridin-2-yl)-1-methyl-1H-pyrazole-4-carboxamide

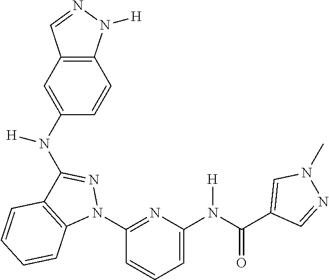 [SMILES: Cn1cc(C(=O)Nc2cccc(-n3nc(Nc4ccc5[nH]ncc5c4)c4ccccc43)n2)cn1]

To a solution of 1-methyl-N-(6-(3-((1-(tetrahydro-2H-pyran-2-yl)-1H-indazol-5-yl)amino)-1H-indazol-1-yl)pyridin-2-yl)-1H-pyrazole-4-carboxamide (110 mg, 206.15 μmol) in DCM (10 mL) was added TFA (2.31 g, 20.26 mmol) at 0° C., the reaction was stirred at r.t. for 12 hr. The reaction solvent was removed under reduced pressure and the crude product was purified by preparative HPLC (36-66% MeCN in H₂O) to give N-(6-(3-((1H-indazol-5-yl)amino)-1H-indazol-1-yl)pyridin-2-yl)-1-methyl-1H-pyrazole-4-carboxamide (28.40 mg, 48.03 mol, 23% yield) as a yellow solid. LC-MS (ES⁺, Method A), 0.56 min, m/z 450.2 [M+H]⁺. ¹H NMR (400 MHz, DMSO-d₆) δ 10.30 (s, 1H), 9.30 (s, 1H), 9.14 (d, J=8.4 Hz, 1H), 8.51 (s, 2H), 8.20 (d, J=8.0 Hz, 1H), 8.15 (s, 1H), 8.08 (s, 1H), 7.95-7.93 (m, 1H), 7.89-7.86 (m, 1H), 7.71-7.68 (m, 2H), 7.63-7.56 (m, 2H), 7.33 (t, J=7.6 Hz, 1H), 3.94 (s, 3H), exchangeable NH not seen.

Compounds prepared in a similar manner to that set out above are given below in Table 12:

TABLE 12

| Example | Structure | LCMS | ¹H NMR |
|---|---|---|---|
| 46 | 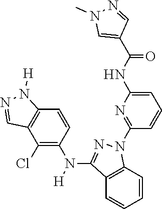 [SMILES: Cn1cc(C(=O)Nc2cccc(-n3nc(Nc4ccc5[nH]ncc5c4Cl)c4ccccc43)n2)cn1] | LC-MS (ES+, Method A), 0.69 min, m/z 484.0 [M + H]⁺ | ¹H NMR (400 MHz, DMSO-d₆) δ 13.36 (s, 1H), 10.28 (s, 1H), 9.10 (d, J = 8.4 Hz, 1H), 8.65 (s, 1H), 8.50 (s, 1H), 8.14 (s, 1H), 8.11 (s, 1H), 8.04 (d, J = 8.0 Hz, 1H), 7.95 (d, J = 8.8 Hz, 1H), 7.83-7.79 (m, 2H), 7.61-7.56 (m, 2H), 7.37-7.34 (m, 1H), 7.28 (t, J = 8.0 Hz, 1H), 3.93 (s, 3H) |
| 47 | 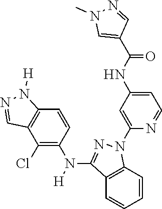 [SMILES: Cn1cc(C(=O)Nc2ccnc(-n3nc(Nc4ccc5[nH]ncc5c4Cl)c4ccccc43)c2)cn1] | LC-MS (ES+, Method A), 0.59 min, m/z 484.1 [M + H]⁺ | ¹H NMR (400 MHz, DMSO-d₆) δ 13.37 (s, 1H), 10.23 (s, 1H), 8.73 (d, J = 8.8 Hz, 1H), 8.63 (s, 1H), 8.38-8.30 (m, 2H), 8.11 (s, 1H), 8.06-7.99 (m, 2H), 7.92 (s, 1H), 7.91- 7.78 (m, 1H), 7.66 (d, J = 5.6 Hz, 1H), 7.67-7.62 (m, 2H), 7.27-7.25 (m, 1H), 3.89 (s, 3H) |

### General Route or the Synthesis of Examples 48-49:

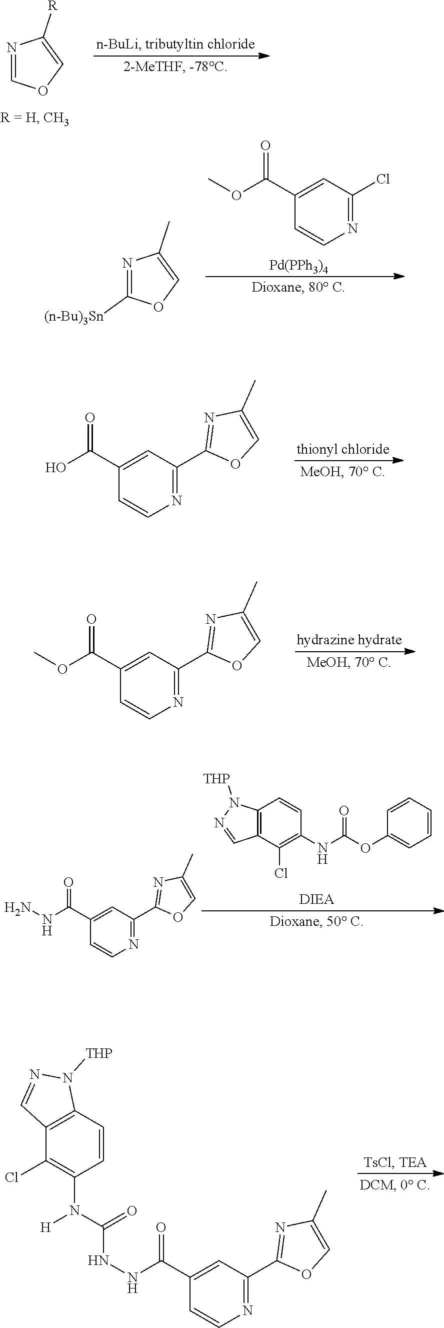

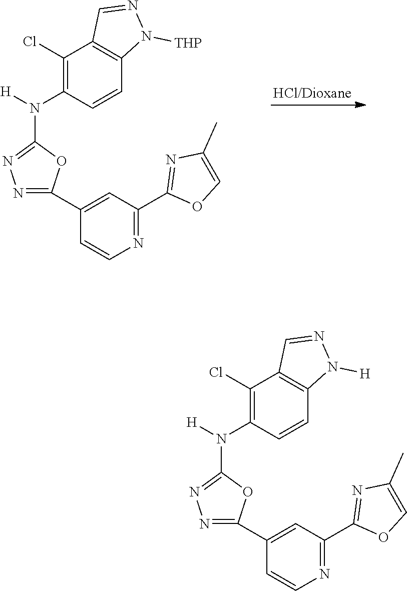

### Step 1: 4-methyl-2-(tributylstannyl)oxazole

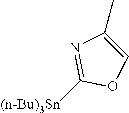

To a solution of 4-methyloxazole (2 g, 24.07 mmol) in 2-MeTHF (80 mL) at −78° C. under nitrogen was added n-BuLi (9.63 mL, 24.07 mmol, 2.5 M) slowly and the reaction was stirred at −78° C. for 0.5 hr. Then the tributyltin chloride (7.84 g, 24.07 mmol) was added. The reaction was allowed to warm to r.t. and stirred for 1 hr under nitrogen. The reaction mixture solvent was removed under reduced pressure. Then the residue was suspended into Petroleum ether (60 mL). The resulting precipitate was filtered and the filtrate was removed under reduced pressure to give 4-methyl-2-(tributylstannyl)oxazole (8 g, 21.5 mmol, 89% yield) as a colorless liquid.

### Step 2: 2-(4-methyloxazol-2-yl)isonicotinic acid

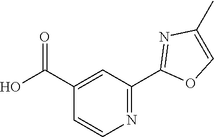

To a solution of methyl 2-chloroisonicotinate (461 mg, 2.69 mmol) and 4-methyl-2-(tributylstannyl)oxazole (5 g, 13.44 mmol) in dioxane (25 mL) was added Pd(PPh₃)₄ (311 mg, 268.72 μmol) at r.t. The reaction was evacuated, flushed with nitrogen and stirred at 80° C. for 12 hr. The reaction mixture was quenched by addition saturated KF aqueous solution (80 mL) at 0° C. and the aqueous phase was extracted with EtOAc (2×80 mL). The combined organics were washed with washed with brine (2×100 mL), dried over sodium sulfate, filtered and the solvent was removed under reduced pressure. The residue was loaded onto silica and purified by column chromatography eluting with 0-30% EtOAc in Pet. Ether to give methyl 2-(4-methyloxazol-2-yl)isonicotinate (40 mg, 183.31 nol, 6.8% yield) as a white solid and the aqueous phase was purified by reversed phase flash (30-80% MeCN in H₂O) to give 2-(4-methyloxazol-2-yl)isonicotinic acid (200 mg, 832.59 μmol, 31% yield) as a white solid. LC-MS (ES⁺, Method A), 0.28 min, m/z 205.1 [M+H]⁺.

### Step 3: methyl 2-(4-methyloxazol-2-yl)isonicotinate

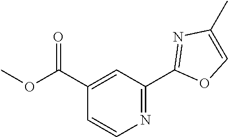

To a solution of 2-(4-methyloxazol-2-yl)isonicotinic acid (200 mg, 1.05 mmol) in MeOH (10 mL) was added thionyl chloride (250 mg, 2.10 mmol) at 0° C., and then the reaction was stirred at 70° C. for 12 hr. It was then cooled to r.t. and solvent removed under reduced pressure. The residue was loaded onto silica and purified by column chromatography eluting with 0-35% EtOAc in Pet. Ether to give methyl 2-(4-methyloxazol-2-yl)isonicotinate (50 mg, 229.14 μmol, 22% yield) as a white solid. LC-MS (ES⁺, Method A), 0.41 min, m/z 219.0 [M+H]⁺.

### Step 4: 2-(4-methyloxazol-2-yl)isonicotinohydrazide

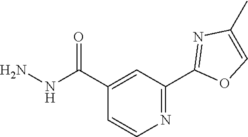 [SMILES: Cc1coc(-c2cc(C(=O)NN)ccn2)n1]

To a solution of methyl 2-(4-methyloxazol-2-yl)isonicotinate (90 mg, 412.45 μmol) in MeOH (2 mL) was added hydrazine hydrate (211 mg, 4.12 mmol) in one portion at r.t. The reaction was stirred at 70° C. for 2 hr. It was then cooled to r.t. and solvent removed under reduced pressure to give 2-(4-methyloxazol-2-yl)isonicotinohydrazide (80 mg, 366.62 μmol, 89% yield) as a yellow solid. LC-MS (ES⁺, Method A), 0.24 min, m/z 219.0 [M+H]⁺.

### Step 5: N-(4-chloro-1-(tetrahydro-2H-pyran-2-yl)-1H-indazol-5-yl)-2-(2-(4-methyloxazol-2-yl)isonicotinoyl)hydrazinecarboxamide

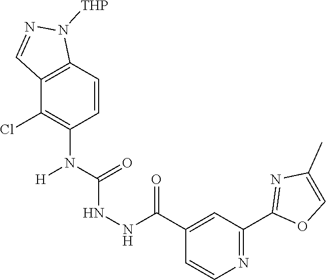

To a solution of 2-(4-methyloxazol-2-yl)isonicotinohydrazide (120 mg, 549.92 μmol) and phenyl (4-chloro-1-(tetrahydro-2H-pyran-2-yl)-1H-indazol-5-yl)carbamate (266 mg, 714.90 μmol) in dioxane (10 mL) was HN added DIEA (213 mg, 1.65 mmol) at r.t. The reaction was stirred at 50° C. for 5 hr. It was then cooled to r.t. and solvent removed under reduced pressure. The crude product was purified by re-crystallization from H₂O (30 mL) at r.t. to give N-(4-chloro-1-(tetrahydro-2H-pyran-2-yl)-1H-indazol-5-yl)-2-(2-(4-methyloxazol-2-yl)isonicotinoyl)hydrazinecarboxamide (200 mg, 403.29 μmol, 73% yield) as a yellow solid. LC-MS (ES⁺, Method A), 0.39 min, m/z 496.3 [M+H]⁺.

### Step 6: N-(4-chloro-1-(tetrahydro-2H-pyran-2-yl)-1H-indazol-5-yl)-5-(2-(4-methyloxazol-2-yl)pyridin-4-yl)-1,3,4-oxadiazol-2-amine

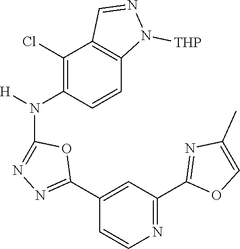

To a solution of N-(4-chloro-1-(tetrahydro-2H-pyran-2-yl)-1H-indazol-5-yl)-2-(2-(4-methyloxazol-2-yl)isonicotinoyl)hydrazinecarboxamide (180 mg, 362.96 mol) in DCM (20 mL) was added TosCl (97 mg, 508.15 μmol) and TEA (110 mg, 1.09μmmol) at 0° C., the reaction was stirred at 0° C. for 2 hr. It was then warmed to r.t. and solvent removed under reduced pressure. The crude product was purified by re-crystallization from MeOH (15 mL) at r.t. to give N-(4-chloro-1-(tetrahydro-2H-pyran-2-yl)-1H-indazol-5-yl)-5-(2-(4-methyloxazol-2-yl)pyridin-4-yl)-1,3,4-oxadiazol-2-amine (130 mg, 272.02 μmol, 75% yield) as a yellow solid. LC-MS (ES⁺, Method A), 0.47 min, m/z 478.2 [M+H]⁺.

### Example 48: N-(4-chloro-1H-indazol-5-yl)-5-(2-(4-methyloxazol-2-yl)pyridin-4-yl)-1,3,4-oxadiazol-2-amine

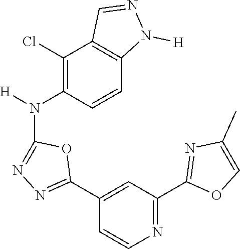 [SMILES: Cc1coc(-c2cc(-c3nnc(Nc4ccc5[nH]ncc5c4Cl)o3)ccn2)n1]

To a solution of N-(4-chloro-1-(tetrahydro-2H-pyran-2-yl)-1H-indazol-5-yl)-5-(2-(4-methyloxazol-2-yl)pyridin-4-yl)-1,3,4-oxadiazol-2-amine (120 mg, 251.10 μmol) was added HCl/dioxane (4 M, 12 mL) at r.t. and the reaction was stirred at r.t. for 2 hr. The reaction solvent removed under reduced pressure. The crude product was triturated with DMSO (3 mL) and H₂O (10 mL) at r.t. for 30 min to give N-(4-chloro-1H-indazol-5-yl)-5-(2-(4-methyloxazol-2-yl)pyridin-4-yl)-1,3,4-oxadiazol-2-amine (13.10 mg, 32.60 μmol, 13% yield) as a yellow solid. LC-MS (ES⁺, Method A), 0.39 min, m/z 394.0 [M+H]⁺. ¹H NMR (400 MHz, DMSO-d₆) δ 13.48 (s, 1H), 10.36 (s, 1H), 8.85 (d, J=4.8 Hz, 1H), 8.38 (s, 1H), 8.15 (s, 1H), 8.05 (d, J=1.2 Hz, 1H), 7.87 (dd, J=4.8 Hz, 1.2 Hz, 1H), 7.80 (d, J=8.8 Hz, 1H), 7.62 (d, J=8.8 Hz, 1H), 2.21 (s, 3H).

Compounds prepared in a similar manner to that set out above are given below in Table 13:

TABLE 13

| Example | Structure | LCMS | ¹H NMR |
|---|---|---|---|
| 49 | 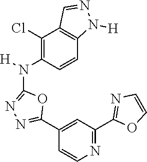 [SMILES: Clc1c(Nc2nnc(-c3ccnc(-c4ncco4)c3)o2)ccc2[nH]ncc12] | LC-MS (ES*, Method A), 0.43 min, m/z 380.1 [M + H]⁺ | ¹H NMR (400 MHz, DMSO-d₆) δ 13.51 (s, 1H), 10.38 (s, 1H), 8.87 (d, J = 3.2 Hz, 1H), 8.40-8.36 (m, 2H), 8.16 (s, 1H), 7.88 (d, J = 2.0 Hz, 1H), 7.78 (d, J = 8.8 Hz, 1H), 7.62 (d, J = 8.8 Hz, 1H), 7.52 (s, 1H) |

### General Route or the Synthesis of Examples 50-51:

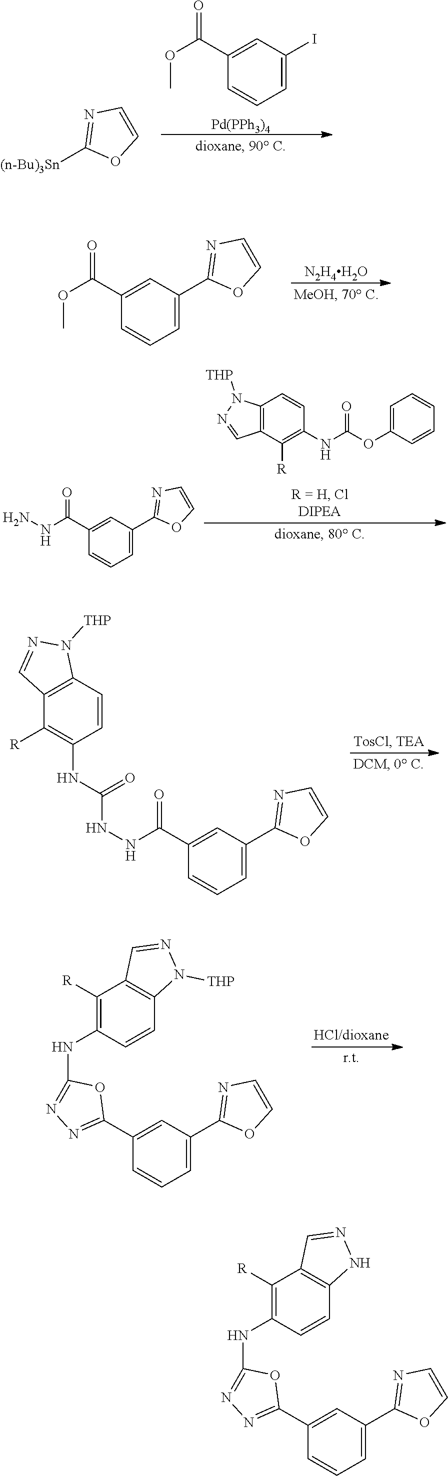

### Step 1: methyl 3-(oxazol-2-yl)benzoate

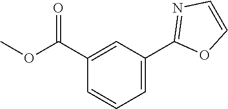

To a solution of 2-(tributylstannyl)oxazole (10 g, 27.92 mmol) and methyl 3-iodobenzoate (2.44 g, 9.31 mmol) in dioxane (80 mL) was added Pd(PPh₃)₄ (1.08 g, 930.67 μmol) at r.t. The reaction was stirred at 90° C. for 12 hr. The reaction was cooled to r.t. and solvent removed under reduced pressure. The residue was partitioned between H₂O (110 mL) and EtOAc (90 mL). The organic layer was separated and the aqueous layer was extracted with EtOAc (2×90 mL). The combined organics were washed with brine (100 mL), dried over sodium sulfate, filtered and the solvent removed under reduced pressure. The residue was loaded onto silica and purified by column chromatography eluting with 0-15% EtOAc in Pet. Ether to give methyl 3-(oxazol-2-yl)benzoate (1 g, 4.92 mmol, 53% yield) as a white solid. LC-MS (ES⁺, Method A), 0.41 min, m/z 204.2 [M+H]⁺.

### Step 2: 3-(oxazol-2-yl)benzohydrazide

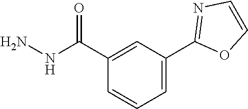 [SMILES: NNC(=O)c1cccc(-c2ncco2)c1]

To a solution of methyl 3-(oxazol-2-yl)benzoate (500 mg, 2.46 mmol) in MeOH (15 mL) was added hydrazine hydrate (1.23 g, 24.08 mmol) in one portion at r.t., and the reaction was stirred at 70° C. for 12 hr. It was then cooled to r.t. and solvent removed under reduced pressure. The crude product was triturated with MeOH (10 mL) at r.t. to give 3-(oxazol-2-yl)benzohydrazide (230 mg, 1.13 mmol, 46% yield) as a white solid. LC-MS (ES⁺, Method A), 0.24 min, m/z 204.1 [M+H]⁺.

### Step 3: N-(4-chloro-1-(tetrahydro-2H-pyran-2-yl)-1H-indazol-5-yl)-2-(3-(oxazol-2-yl)benzoyl)hydrazinecarboxamide

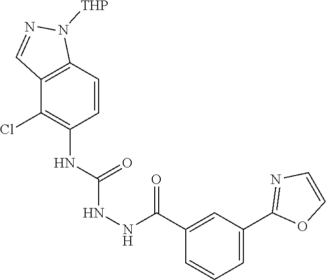

To a solution of 3-(oxazol-2-yl)benzohydrazide (140 mg, 688.99 μmol) and phenyl (4-chloro-1-(tetrahydro-2H-pyran-2-yl)-1H-indazol-5-yl)carbamate (307.41 mg, 826.78 μmol) in dioxane (8 mL) was added DIEA (178.09 mg, 1.38 mmol) at r.t. The reaction was stirred at 80° C. for 5 hr. It was then cooled to r.t. And the mixture was diluted with H₂O (15 mL). The reaction mixture was filtered and the filter cake was washed by EtOAc (3×10 mL) to give N-(4-chloro-1-(tetrahydro-2H-pyran-2-yl)-1H-indazol-5-yl)-2-(3-(oxazol-2-yl)benzoyl)hydrazinecarboxamide (180 mg, 374.30 μmol, 54% yield) as an off-white solid. LC-MS (ES⁺, Method A), 0.48 min, m/z 481.2 [M+H]⁺.

### Step 4: N-(4-chloro-1-(tetrahydro-2H-pyran-2-yl)-1H-indazol-5-yl)-5-(3-(oxazol-2-yl)phenyl)-1,3,4-oxadiazol-2-amine

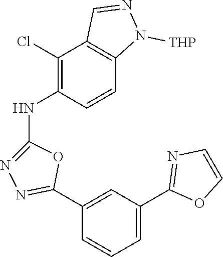

To a solution of N-(4-chloro-1-(tetrahydro-2H-pyran-2-yl)-1H-THP indazol-5-yl)-2-(3-(oxazol-2-yl)benzoyl)hydrazinecarboxamide (260 mg, 540.65 μmol) in DCM (10 mL) was added TEA (164 mg, 1.62 mmol) and TosCl (144 mg, 756.91 μmol) at 0° C., and the reaction was stirred at 0° C. for 4 hr. It was then warmed to r.t. and solvent removed under reduced pressure. The crude product was purified by re-crystallization from MeOH (10 mL) at r.t. to give N-(4-chloro-1-(tetrahydro-2H-pyran-2-yl)-1H-indazol-5-yl)-5-(3-(oxazol-2-yl)phenyl)-1,3,4-oxadiazol-2-amine (122 mg, 263.56 μmol, 49% yield) as an off-white solid.

LC-MS (ES⁺, Method A), 0.69 min, m/z 462.9 [M+H]⁺.

### Example 50: N-(4-chloro-1H-indazol-5-yl)-5-(3-(oxazol-2-yl)phenyl)-1,3,4-oxadiazol-2-amine

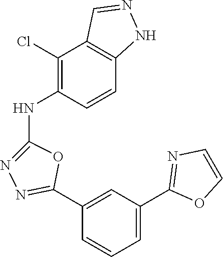 [SMILES: Clc1c(Nc2nnc(-c3cccc(-c4ncco4)c3)o2)ccc2[nH]ncc12]

To a solution of N-(4-chloro-1-(tetrahydro-2H-pyran-2-yl)-1H-CI indazol-5-yl)-5-(3-(oxazol-2-yl)phenyl)-1,3,4-oxadiazol-2-amine (90 mg, 194.43 μmol) was added HCl/dioxane (4 M, 4 mL) at r.t. and then reaction was stirred at r.t. for 12 hr. The reaction mixture was filtered and the filter cake was washed by MeCN (30 mL) and the filter cake was lyophilized to give N-(4-chloro-1H-indazol-5-yl)-5-(3-(oxazol-2-yl)phenyl)-1,3,4-oxadiazol-2-amine (65.40 mg, 170.94 μmol, 88% yield) as a yellow solid. LC-MS (ES⁺, Method A), 0.51 min, m/z 379.2 [M+H]⁺. ¹H NMR (400 MHz, DMSO-d₆) δ 10.16 (s, 1H), 8.43 (s, 1H), 8.30 (s, 1H), 8.14-8.13 (m, 2H), 8.00 (d, J=8.0 Hz, 1H), 7.80 (d, J=8.8 Hz, 1H), 7.74 (t, J=7.6 Hz, 1H), 7.62 (d, J=8.8 Hz, 1H), 7.45 (s, 1H), exchangeable NH not seen.

Compounds prepared in a similar manner to that set out above are given below in Table 14:

TABLE 14

| Example | Structure | LCMS | ¹H NMR |
|---|---|---|---|
| 51 | 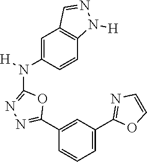 [SMILES: c1cc(-c2ncco2)cc(-c2nnc(Nc3ccc4[nH]ncc4c3)o2)c1] | LC-MS (ES+, Method A), 0.40 min, m/z 345.2 [M + H]⁺ | ¹H NMR (400 MHz, CD-C, DMSO-d₆) δ 10.75 (s, 1H), 8.48 (s, 1H), 8.31 (s, 1H), 8.17-8.14 (m, 2H), 8.08 (s, 1H), 8.04 (d, J = 7.6 Hz, 1H), 7.76 (t, J = 8.0 Hz, 1H), 7.58-7.51 (m, 1H), 7.51-7.48 (m, 1H), 7.46 (s, 1H), exchangeable NH not seen |

Method for the synthesis of Example 52:

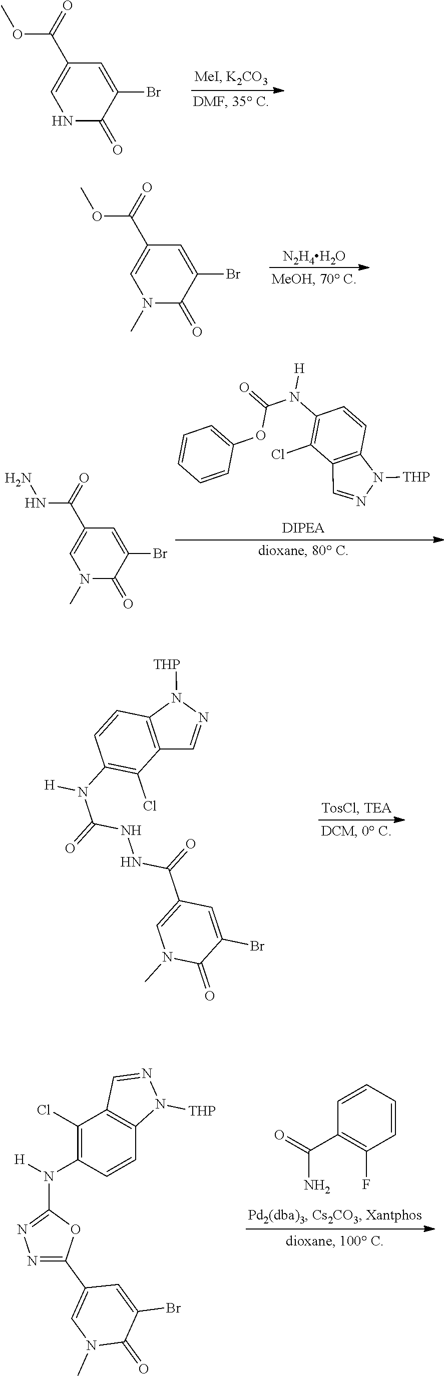

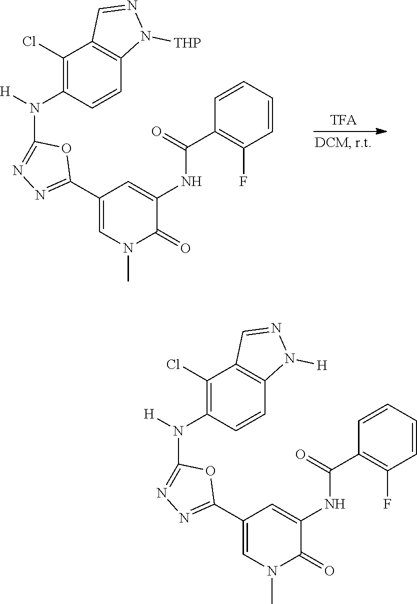

### Step 1: methyl 5-bromo-1-methyl-6-oxo-1,6-dihydropyridine-3-carboxylate

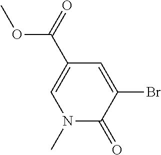

To a mixture of methyl 5-bromo-6-oxo-1,6-dihydropyridine-3-carboxylate (4.98 g, 21.46 mmol) and K₂CO₃ (5.93 g, 42.93 mmol) in DMF (50 Br mL) was added Mel (4.57 g, 32.19 mmol) slowly at 25° C., and the mixture was stirred at 35° C. for 16 hr. After cooled to room temperature, the reaction mixture was diluted with ethyl acetate (600 mL) and then washed with water (200 mL) and brine (200 mL). The organic layer was dried over sodium sulfate, filtered and concentrated under reduced pressure to give methyl 5-bromo-1-methyl-6-oxo-1,6-dihydropyridine-3-carboxylate (5.2 g, 21.13 mmol) as an off-white solid.

### Step 2: 5-bromo-1-methyl-6-oxo-1,6-dihydropyridine-3-carbohydrazide

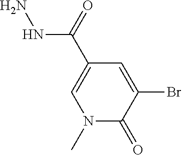

To a solution of methyl 5-bromo-1-methyl-6-oxo-1,6-HNJ dihydropyridine-3-carboxylate (1 g, 4.06 mmol) in MeOH (10 mL) was Br added N₂H4.H₂O (1.04 g, 20.32 mmol) in one portion at 25° C., and then the mixture was stirred at 70° C. for 4 hr. The reaction solution was diluted with methanol (30 mL), concentrated under reduced to give 5-bromo-1-methyl-6-oxo-1,6-dihydropyridine-3-carbohydrazide (950 mg, 3.86 mmol, 95% yield) as a yellow solid. LC-MS (ES⁺, Method D), 0.17 min, m/z 246.0 [M+H]⁺.

### Step 3: 2-(5-bromo-1-methyl-6-oxo-1,6-dihydropyridine-3-carbonyl)-N-(4-chloro-1-(tetrahydro-2H-pyran-2-yl)-1H-indazol-5-yl)hydrazinecarboxamide

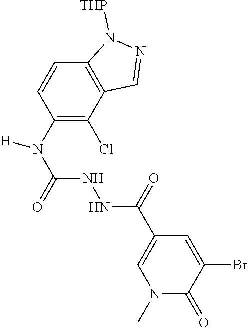

To a solution of 5-bromo-1-methyl-6-oxo-1,6-dihydropyridine-3-carbohydrazide (395 mg, 1.61 mmol) and phenyl N-(4-chloro-1-tetrahydropyran-2-yl-indazol-5-yl)carbamate (596.9 mg, 1.61 mmol) in dioxane (12 mL) was added DIPEA (415 mg, 3.21 mmol) in one portion at r.t., and the reaction solution was stirred at 80° C. for 16 hr. The mixture was diluted with water (15 mL), and the reaction solution was filtered and the filter cake was washed by ethyl acetate (3×10 mL). The filter cake was collected to give 2-(5-bromo-1-methyl-6-oxo-1,6-dihydropyridine-3-carbonyl)-N-(4-chloro-1-(tetrahydro-2H-pyran-2-yl)-1H-indazol-5-yl)hydrazinecarboxamide (430 mg, 820.98 μmol, 51% yield) as an off-white solid. LC-MS (ES⁺, Method A), 0.46 min, m/z 525.3 [M+H]⁺.

### Step 4: 3-bromo-5-(5-((4-chloro-1-(tetrahydro-2H-pyran-2-yl)-1H-indazol-5-yl)amino)-1,3,4-oxadiazol-2-yl)-1-methylpyridin-2(1H)-one

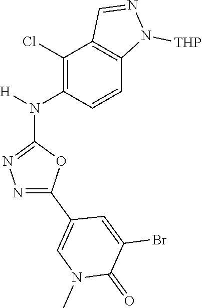

To a solution of 2-(5-bromo-1-methyl-6-oxo-1,6-dihydropyridine-3-carbonyl)-N-(4-chloro-1-(tetrahydro-2H-pyran-2-yl)-H 1H-indazol-5-yl)hydrazinecarboxamide (430 mg, 820.98 μmol) in DCM (12 mL) was added TosCl (219.12 mg, 1.15 mmol) and TEA (249.22 mg, 2.46 mmol) at 0° C., and the reaction solution was stirred at 0° C. for 2 hr. The reaction solution was filtered and the filter cake was washed by DMF (16 mL) and the filter cake was collected to give 3-bromo-5-(5-((4-chloro-1-(tetrahydro-2H-pyran-2-yl)-1H-indazol-5-yl)amino)-1,3,4-oxadiazol-2-yl)-1-methylpyridin-2(1H)-one (180 mg, 355.91 μmol, 43% yield) as an off-white solid. LC-MS (ES⁺, Method A), 0.50 min, m/z 507.0 [M+H]⁺.

### Step 5: N-(5-(5-((4-chloro-1-(tetrahydro-2H-pyran-2-yl)-1H-indazol-5-yl)amino)-1,3,4-oxadiazol-2-yl)-1-methyl-2-oxo-1,2-dihydropyridin-3-yl)-2-fluorobenzamide

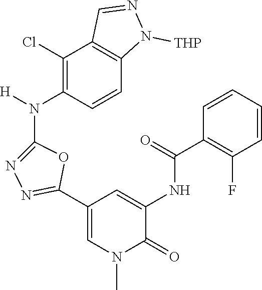

To a mixture of 3-bromo-5-(5-((4-chloro-1-(tetrahydro-2H-pyran-2-yl)-1H-indazol-5-yl)amino)-1,3,4-oxadiazol-2-yl)-1-methylpyridin-2(1H)-one (180 mg, 355.91 μmol) and 2-fluorobenzamide (99 mg, 711.81 μmol) in dioxane (3 mL) was added Cs₂CO₃ (231.9 mg, 711.81 μmol), Xantphos (41.2 mg) and Pd₂(dba)₃ (32.6 mg, 35.59 μmol) in one portion at r.t. under N₂, and then the mixture was stirred at 100° C. for 16 h under N₂. After cooled to room temperature, the reaction mixture was diluted with ethyl acetate (200 mL) and then washed with water (50 mL) and brine (50 mL). The organic layer was dried over sodium sulfate, filtered and concentrated under reduced pressure to give N-(5-(5-((4-chloro-1-(tetrahydro-2H-pyran-2-yl)-1H-indazol-5-yl)amino)-1,3,4-oxadiazol-2-yl)-1-methyl-2-oxo-1,2-dihydropyridin-3-yl)-2-fluorobenzamide (340 mg, crude) as an off-white solid. LC-MS (ES⁺, Method A), 0.63 min, m/z 564.4 [M+H]⁺.

### Example 52: N-(5-(5-((4-chloro-1H-indazol-5-yl)amino)-1,3,4-oxadiazol-2-yl)-1-methyl-2-oxo-1,2-dihydropyridin-3-yl)-2-fluorobenzamide

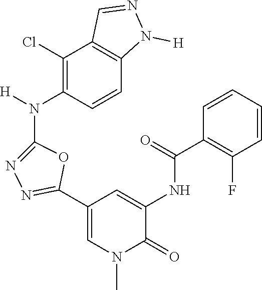 [SMILES: Cn1cc(-c2nnc(Nc3ccc4[nH]ncc4c3Cl)o2)cc(NC(=O)c2ccccc2F)c1=O]

To a solution of N-(5-(5-((4-chloro-1-(tetrahydro-2H-pyran-CI 2-yl)-1H-indazol-5-yl)amino)-1,3,4-oxadiazol-2-yl)-1-methyl-2-oxo-H,N 1,2-dihydropyridin-3-yl)-2-fluorobenzamide (320 mg, 567.41 μmol) in DCM (5 mL) was added TFA (7.70 g, 67.53 mmo) in one portion, and the reaction solution was stirred at r.t. for 1 hr. The reaction solution was diluted with dichloromethane (5 mL) and then concentrated under reduced pressure to give a crude. The crude product was purified by re-crystallized with acetonitrile (10 mL) at 80° C. to give N-(5-(5-((4-chloro-1H-indazol-5-yl)amino)-1,3,4-oxadiazol-2-yl)-1-methyl-2-oxo-1,2-dihydropyridin-3-yl)-2-fluorobenzamide (31.40 mg, 62.17 μmol, 75% yield) as a yellow solid. LC-MS (ES⁺, Method A), 0.48 min, m/z 480.1 [M+H]⁺. ¹H NMR (400 MHz, DMSO-d₆) δ 13.07 (s, 1H), 9.77 (d, J=11.2 Hz, 1H), 8.89 (d, J=2.0 Hz, 1H), 8.12-8.00 (m, 3H), 7.93 (s, 1H), 7.73-7.67 (m, 1H), 7.48-7.39 (m, 4H), 3.65 (s, 3H).

Method for the synthesis of Example 53:

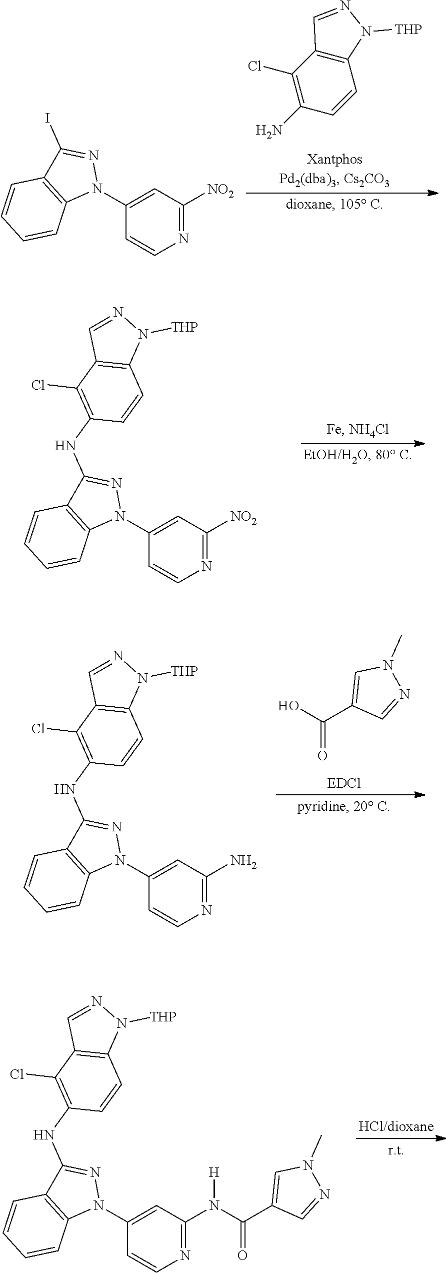

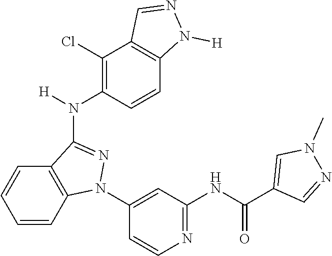 [SMILES: Cn1cc(C(=O)Nc2cc(-n3nc(Nc4ccc5[nH]ncc5c4Cl)c4ccccc43)ccn2)cn1]

### Step 1: N-(4-chloro-1-(tetrahydro-2H-pyran-2-yl)-1H-indazol-5-yl)-1-(2-nitropyridin-4-yl)-1H-indazol-3-amine

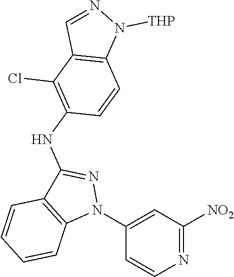

To a mixture of 4-chloro-1-(tetrahydro-2H-pyran-2-yl)-1H-indazol-5-amine (281.26 mg, 1.12 mmol,) and 3-iodo-1-(2-nitropyridin-/4-yl)-1H-indazole (450 mg, 1.23 mmol) in dioxane (20 mL) was added Xantphos (129.31 mg, 223.48 μmol), Pd₂(dba)₃ (102.32 mg, 111.74 μmol) and Cs₂CO₃ (910.17 mg, 2.79 mmol) at r.t. The reaction was evacuated, flushed with nitrogen and stirred at 105° C. for 4 hr. It was cooled to r.t. and the reaction was partitioned between H₂O (30 mL) and EtOAc (40 mL). The organic layer was separated and the aqueous layer was extracted with EtOAc (2×40 mL). The combined organics were washed with brine (2×50 mL), dried over sodium sulfate, filtered and the solvent removed under reduced pressure. The residue was loaded onto silica and purified by column chromatography eluting with 0-25% EtOAc in Pet. Ether to give N-(4-chloro-1-(tetrahydro-2H-pyran-2-yl)-1H-indazol-5-yl)-1-(2-nitropyridin-4-yl)-1H-indazol-3-amine (350 mg, 714.41 μmol, 64% yield) as a red solid. LC-MS (ES⁺, Method A), 0.60 min, m/z 490.2 [M+H]⁺.

### Step 2: 1-(2-aminopyridin-4-yl)-N-(4-chloro-1-(tetrahydro-2H-pyran-2-yl)-1H-indazol-5-yl)-1H-indazol-3-amine

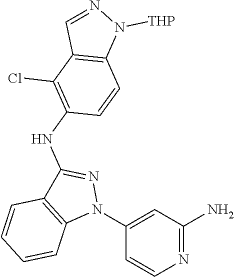

To a solution of N-(4-chloro-1-(tetrahydro-2H-pyran-2-yl)—1H-indazol-5-yl)-1-(2-nitropyridin-4-yl)-1H-indazol-3-amine (150 mg, 306.18 μmol) in EtOH (8 mL) and water (2 mL) was added NH₄Cl (163.78 mg, 3.06 mmol) and Fe (51.30 mg, 918.53 [tmol) at r.t. The reaction was stirred at 80° C. for 2 hr. The reaction mixture was filtered and the filter cake was washed by MeOH (50 mL). The filtrate solvent removed under reduced pressure. The crude product was triturated with H₂O (20 ml) at r.t. for 10 min. The mixture was filtered and the filter cake was washed by H₂O (10 mL) to give 1-(2-aminopyridin-4-yl)-N-(4-chloro-1-(tetrahydro-2H-pyran-2-yl)-1H-indazol-5-yl)-1H-indazol-3-amine (110 mg, 239.17 μmol, 78% yield) as an off-white solid. LC-MS (ES⁺, Method A), 0.50 min, m/z 460.1 [M+H]⁺.

### Step 3: N-(4-(3-((4-chloro-1-(tetrahydro-2H-pyran-2-yl)-1H-indazol-5-yl)amino)-1H-indazol-1-yl)pyridin-2-yl)-1-methyl-1H-pyrazole-4-carboxamide

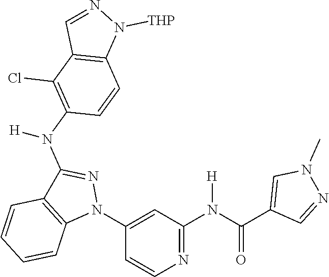

To a solution of 1-(2-aminopyridin-4-yl)-N-(4-chloro-1-(tetrahydro-2H-pyran-2-yl)-1H-indazol-5-yl)-1H-indazol-3-amine (100 mg, 217.42 μmol) and 1-methyl-1H-pyrazole-4-carboxylic acid (55 mg, 434.85 μmol) in pyridine (5 mL) was added EDCI (104 mg, 543.56 μmol) at r.t. and the reaction was stirred at r.t. for 48 hr. The residue was partitioned between H₂O (20 mL) and DCM (20 mL). The organic layer was separated and the aqueous layer was extracted with DCM (2×20 mL). The combined organics were washed with brine (2×30 mL), dried over sodium sulfate, filtered and the solvent removed under reduced pressure. The crude product was purified by reversed phase flash (0-33% MeCN in H₂O) to give N-(4-(3-((4-chloro-1-(tetrahydro-2H-pyran-2-yl)-1H-indazol-5-yl)amino)-1H-indazol-1-yl)pyridin-2-yl)-1-methyl-1H-pyrazole-4-carboxamide (100 mg, 156.68 μmol, 72% yield) as a brown solid. LC-MS (ES⁺, Method A), 0.53 min, m/z 568.2 [M+H]⁺.

### Example 53: N-(4-(3-((4-chloro-1H-indazol-5-yl)amino)-1H-indazol-1-yl)pyridin-2-yl)-1-methyl-1H-pyrazole-4-carboxamide

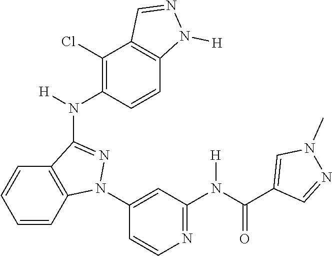 [SMILES: Cn1cc(C(=O)Nc2cc(-n3nc(Nc4ccc5[nH]ncc5c4Cl)c4ccccc43)ccn2)cn1]

To a solution of N-(4-(3-((4-chloro-1-(tetrahydro-2H-pyran-2-yl)-1H-indazol-5-yl)amino)-1H-indazol-1-yl)pyridin-2-/yl)-1-methyl-1H-pyrazole-4-carboxamide (95 mg, 167.25 μmol) was added HCl/dioxane (4 M, 4 mL) at r.t. The reaction stirred at r.t. for 12 h. The reaction mixture was filtered and filter cake was washed by MeCN (30 mL). The crude product was purified by re-crystallization from MeCN (15 mL) at 45° C. to give N-(4-(3-((4-chloro-1H-indazol-5-yl)amino)-1H-indazol-1-yl)pyridin-2-yl)-1-methyl-1H-pyrazole-4-carboxamide (20.30 mg, 41.53 μmol, 25% yield) as a green solid. LC-MS (ES⁺, Method A), 0.47 min, m/z 484.2 [M+H]⁺. ¹H NMR (400 MHz, DMSO-d₆) δ 11.96 (s, 1H), 9.13 (s, 1H), 8.66 (s, 1H), 8.41 (s, 1H), 8.34-8.28 (m, 3H), 8.24 (d, J=8.0 Hz, 1H), 8.15 (s, 1H), 7.86 (d, J=8.8 Hz, 1H), 7.75-7.71 (m, 2H), 7.63 (d, J=8.8 Hz, 1H), 7.47 (t, J=7.2 Hz, 1H), 3.93 (s, 3H), exchangeable NH not seen.

Method for the synthesis of Example 54:

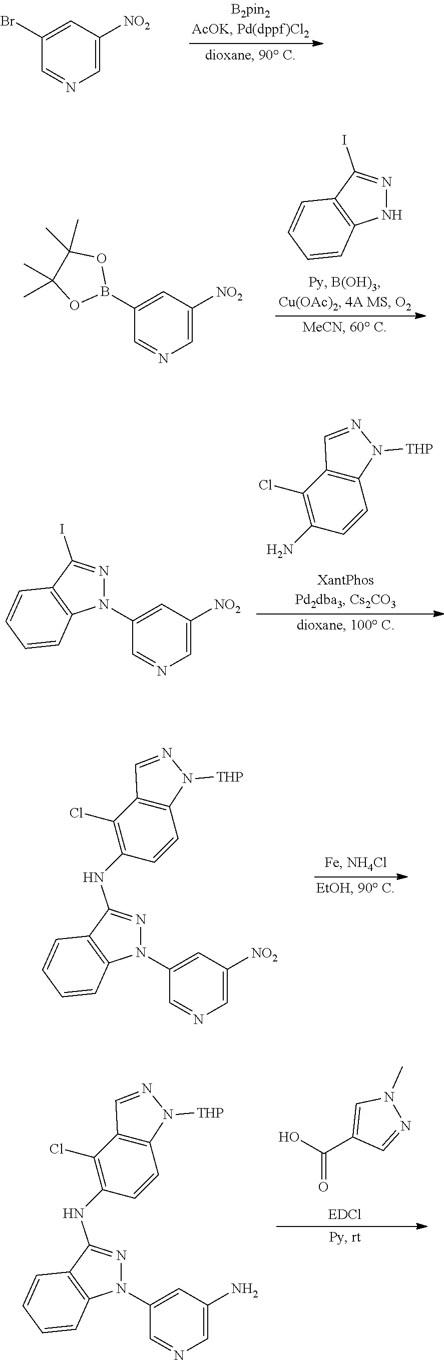

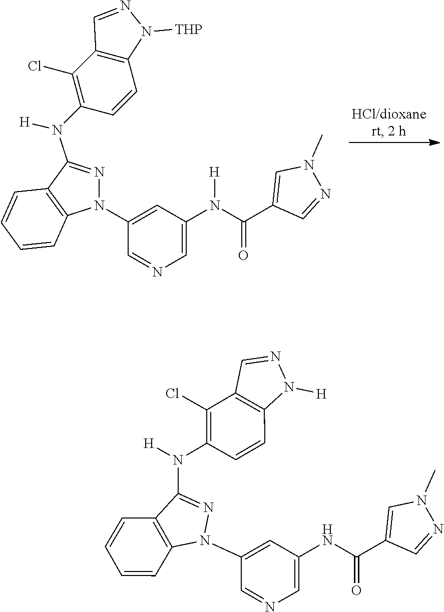

### Step 1: 3-nitro-5-(4,4,5,5-tetramethyl-1,3,2-dioxaborolan-2-yl)pyridine

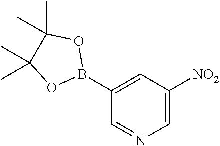 [SMILES: CC1(C)OB(c2cncc([N+](=O)[O-])c2)OC1(C)C]

To a solution of 3-bromo-5-nitropyridine (3 g, 14.78 mmol) intense dioxane (100 mL) was added 4,4,4′,4′,5,5,5′,5′-octamethyl-2,2′-bi(1,3,2-dioxaborolane) (7.51 g, 29.56 mmol), Pd(dppf)Cl₂ (540 mg, 738.94 μmol) and AcOK (4.35 g, 44.34 mmol). The reaction was evacuated, flushed with nitrogen and stirred at 90° C. for 12 h. The reaction was cooled to r.t. and the mixture was partitioned between H₂O (100 mL) and EtOAc (75 mL). The organic layer was separated and the aqueous layer was extracted with EtOAc (3×75 mL). The combined organics were washed with washed with brine (2×75 mL), dried over sodium sulfate, filtered and the solvent removed under reduced pressure. The residue was loaded onto silica and purified by column chromatography eluting with 0-20% EtOAc in Pet. Ether to give 3-nitro-5-(4,4,5,5-tetramethyl-1,3,2-dioxaborolan-2-yl)pyridine (3.5 g, 14.00 mmol, 94.5% yield) as yellow solid.

### Step 2: 3-iodo-1-(5-nitropyridin-3-yl)-1H-indazole

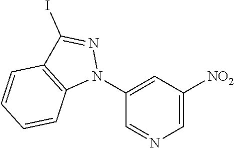 [SMILES: O=[N+]([O-])c1cncc(-n2nc(I)c3ccccc32)c1]

The solution of 3-nitro-5-(4,4,5,5-tetramethyl-1,3,2-dioxaborolan-2-yl)pyridine (500 mg, 2.00 mmol) and 3-iodo-1H-indazole (488 mg, 2.00 mmol) in MeCN (80 mL) was added pyridine (316 mg, 4.00 mmol), Cu(OAc)₂ (545 mg, 3.00 mmol), boric acid (247 mg, 4.00 mmol) and 200 mg 4 Å molecular sieve, and the reaction solution was bubbled with air and stirred at 60° C. for 12 hr. The reaction was cooled to r.t. and solvent removed under reduced pressure. The residue was partitioned between H₂O (200 mL) and EtOAc (50 mL). The organic layer was separated and the aqueous layer was extracted with EtOAc (3×50 mL). The combined organics were washed with washed with brine (2×20 mL), dried over sodium sulfate, filtered and the solvent removed under reduced pressure. The residue was loaded onto silica and purified by column chromatography eluting with 0-15% EtOAc in Pet. Ether to give 3-iodo-1-(5-nitropyridin-3-yl)-1H-indazole (270 mg, 737.48 μmol, 37% yield) as a yellow solid. LC-MS (ES⁺, Method A), 0.58 min, m/z 367.0 [M+H]⁺.

### Step 3: N-(4-chloro-1-(tetrahydro-2H-pyran-2-yl)-1H-indazol-5-yl)-1-(5-nitropyridin-3-yl)-1H-indazol-3-amine

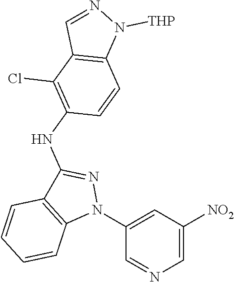

To a solution of 3-iodo-1-(5-nitropyridin-3-yl)-1H-indazole 260 mg, 710.16 μmol) and 4-chloro-1-(tetrahydro-2H-pyran-2-yl)-1H-indazol-5-amine (179 mg, 710.16 μmol) in dioxane (6 mL) was added Xantphos (82 mg, 142.03 μmol), Cs₂CO₃ (463 mg, 1.42 mmol) and Pd₂(dba)₃ (65 mg, 71.02 μmol) at r.t. The reaction was evacuated, flushed with nitrogen and stirred at 100° C. for 2 hr. The reaction was cooled to r.t. and solvent removed under reduced pressure. The residue was partitioned between H₂O (20 mL) and EtOAc (10 mL). The organic layer was separated and the aqueous layer was extracted with EtOAc (3×10 mL). The combined organics were washed with washed with brine (2×10 mL), dried over sodium sulfate, filtered and the solvent removed under reduced pressure. The residue was loaded onto silica and purified by column chromatography eluting with 0-20% EtOAc in Pet. Ether to give N-(4-chloro-1-(tetrahydro-2H-pyran-2-yl)-1H-indazol-5-yl)-1-(5-nitropyridin-3-yl)-1H-indazol-3-amine (270 mg, 551.12 μmol, 78% yield) as a red solid. LC-MS (ES⁺, Method A), 0.66 min, m/z 490.1 [M+H]⁺.

### Step 4: 1-(5-aminopyridin-3-yl)-N-(4-chloro-1-(tetrahydro-2H-pyran-2-yl)-1H-indazol-5-yl)-1H-indazol-3-amine

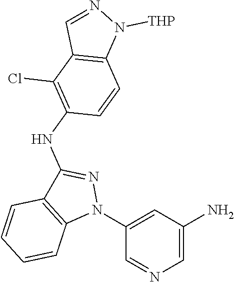

To a solution of N-(4-chloro-1-(tetrahydro-2H-pyran-2-yl)-1H-indazol-5-yl)-1-(5-nitropyridin-3-yl)-1H-indazol-3-amine (200 mg, 314.34 μmol) in EtOH (16 mL) and H₂O (4 mL) was added NH₄Cl (168 mg, 3.14 mmol) and Fe (53 mg, 943.02 μmol) at r.t. the reaction was N stirred at 80° C. for 2 hr. Then the reaction mixture was filtered and the filter cake was washed by MeOH (70 mL). The filtrate solvent removed under reduced pressure and the crude product was triturated with H₂O (20 mL) at r.t. for 10 minutes. The mixture was filtered and the filter cake was washed by H₂O (10 mL) to give 1-(5-aminopyridin-3-yl)-N-(4-chloro-1-(tetrahydro-2H-pyran-2-yl)-1H-indazol-5-yl)-1H-indazol-3-amine (120 mg, 260.91 μmol, 83% yield) as a red solid. LC-MS (ES⁺, Method A), 0.46 min, m/z 460.2 [M+H]⁺.

### Step 5: N-(5-(3-((4-chloro-1-(tetrahydro-2H-pyran-2-yl)-1H-indazol-5-yl)amino)-1H-indazol-1-yl)pyridin-3-yl)-1-methyl-1H-pyrazole-4-carboxamide

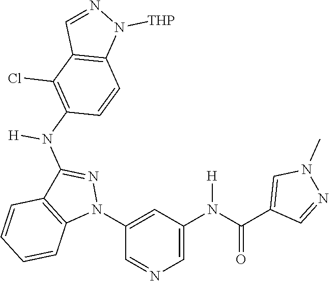

To a solution of 1-(5-aminopyridin-3-yl)-N-(4-chloro-1-(tetrahydro-2H-pyran-2-yl)-1H-indazol-5-yl)-1H-indazol-3-amine (200 mg, 434.85 μmol) and 1-methyl-1H-pyrazole-4-carboxylic acid (165 mg, 1.30 mmol) in pyridine (10 mL) was added EDCI (333 mg, 1.74 mmol) at r.t. and the reaction was stirred at r.t. for 12 hr. The solvent was removed under reduced pressure and the residue was purified by reversed phase flash (0-60% MeCN in H₂O) to give N-(5-(3-((4-chloro-1-(tetrahydro-2H-pyran-2-yl)-1H-indazol-5-yl)amino)-1H-indazol-1-yl)pyridin-3-yl)-1-methyl-1H-pyrazole-4-carboxamide (100 mg, 176.05 mol, 40% yield) as a red solid. LC-MS (ES⁺, Method A), 0.48 min, m/z 568.4 [M+H]⁺.

### Example 54: N-(5-(3-((4-chloro-1H-indazol-5-yl)amino)-1H-indazol-1-yl)pyridin-3-yl)-1-methyl-1H-pyrazole-4-carboxamide

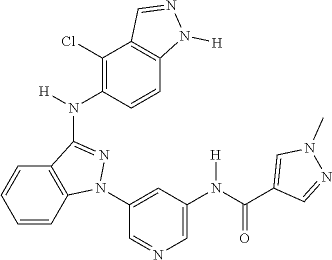 [SMILES: Cn1cc(C(=O)Nc2cncc(-n3nc(Nc4ccc5[nH]ncc5c4Cl)c4ccccc43)c2)cn1]

solution of N-(5-(3-((4-chloro-1-(tetrahydro-2H-pyran-2-yl)-1H-indazol-5-yl)amino)-1H-indazol-1-yl)pyridin-3-yl)-1-methyl-1H-pyrazole-4-carboxamide (90 mg, 158.44 mol) was added HCl/dioxane (4 M, 6 mL) was stirred at r.t. for 2 h. The solvent removed under reduced pressure and the crude product was purified by re-crystallization from MeOH (10 mL) at r.t. to give N-(5-(3-((4-chloro-1H-indazol-5-yl)amino)-1H-indazol-1-yl)pyridin-3-yl)-1-methyl-1H-pyrazole-4-carboxamide (42.90 mg, 85.73 mol, 54% yield) as a brown solid. LC-MS (ES⁺, Method A), 0.43 min, m/z 484.2 [M+H]⁺. ¹H NMR (400 MHz, DMSO-d₆) δ 10.9 (s, 1H), 9.04 (d, J=1.6 Hz, 1H), 8.94 (s, 1H), 8.81-8.77 (m, 2H), 8.51 (s, 1H), 8.17 (s, 1H), 8.14 (d, J=8.0 Hz, 1H), 8.13-8.10 (m, 1H), 8.08 (d, J=8.4 Hz, 1H), 7.88 (d, J=8.0 Hz, 1H), 7.65-7.58 (m, 2H), 7.33 (t, J=7.2 Hz, 1H), 3.91 (s, 3H), exchangeable NH not seen.

Method for the synthesis of Example 55:

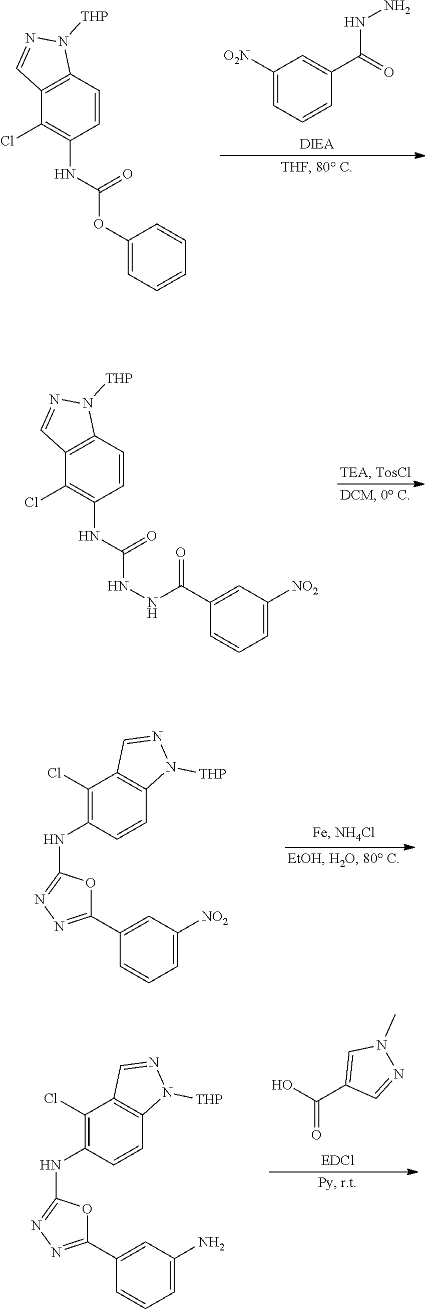

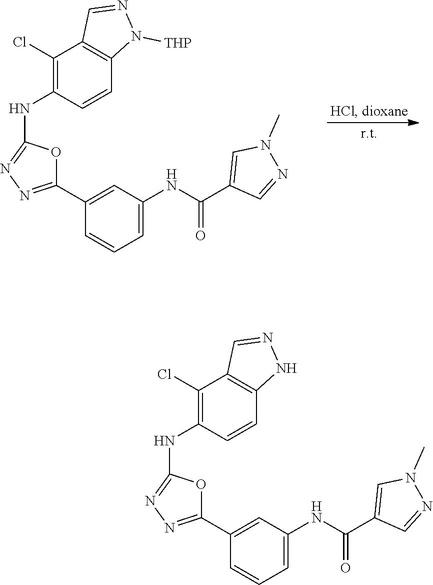

### Step 1: N-(4-chloro-1-(tetrahydro-2H-pyran-2-yl)-1H-indazol-5-yl)-2-(3-nitrobenzoyl)hydrazinecarboxamide

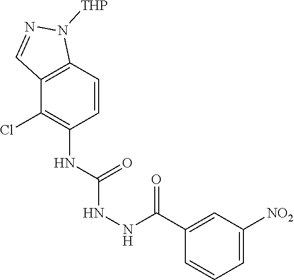

To a mixture of 3-nitrobenzohydrazide (162.4 mg, 896.50 μmol) and phenyl (4-chloro-1-(tetrahydro-2H-pyran-2-yl)-1H-indazol-5-yl)carbamate (400 mg, 1.08 mmol) in THF (5 mL) was added DIEA (347.6 mg, 2.69 mmol) at r.t., and then the mixture was stirred at 80° C. for 4 hr. The reaction solution was concentrated under reduced pressure to give a crude product. The crude product was purified by re-crystallization from H₂O (30 mL) at r.t. to give N-(4-chloro-1-(tetrahydro-2H-pyran-2-yl)-1H-indazol-5-yl)-2-(3-nitrobenzoyl)hydrazinecarboxamide (350 mg, 762.77 μmol, 85.1% yield) as a red solid.

### Step 2: N-(4-chloro-1-(tetrahydro-2H-pyran-2-yl)-1H-indazol-5-yl)-5-(3-nitrophenyl)-1,3,4-oxadiazol-2-amine

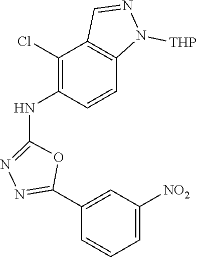

To a mixture of N-(4-chloro-1-(tetrahydro-2H-pyran-2-yl)-1H-THP indazol-5-yl)-2-(3-nitrobenzoyl)hydrazinecarboxamide (340 mg, 740.98 μmol) in DCM (5 mL) was added TosCl (197.8 mg, 1.04 mmol), TEA (224.9 mg, 2.22 mmol) at r.t., and then the mixture was stirred at 0° C. for 1 hr. The mixture was concentrated under reduced pressure to give a crude product. The crude product was purified by re-crystallization from methanol (35 mL) at r.t. to give N-(4-chloro-1-(tetrahydro-2H-pyran-2-yl)-1H-indazol-5-yl)-5-(3-nitrophenyl)-1,3,4-oxadiazol-2-amine (260 mg, 589.78 μmol, 79.6% yield) as a light yellow solid.

### Step 8: 5-(3-aminophenyl)-N-(4-chloro-1-(tetrahydro-2H-pyran-2-yl)-1H-indazol-5-yl)-1,3,4-oxadiazol-2-amine

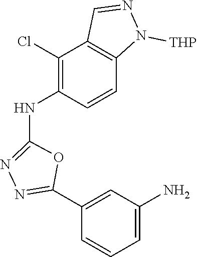

To a mixture of N-(4-chloro-1-(tetrahydro-2H-pyran-2-yl)-1H-CI indazol-5-yl)-5-(3-nitrophenyl)-1,3,4-oxadiazol-2-amine (250 mg, 567.10 μmol) in EtOH (4 mL) was added Fe (95 mg, 1.70 mmol), NH₄Cl (303.4 mg, 5.67 mmol) and H₂O (1 mL) at 20° C., and then the mixture was stirred at 80° C. for 2 hr. The reaction solution was filtered, the filter cake was washed by MeOH (10 mL). The filtrate was concentrated under reduced pressure to give a crude product. The crude product was triturated with H₂O (20 mL) at 25° C. for 10 min and then was filtered, and the filter cake was washed by H₂O (10 mL) to give 5-(3-aminophenyl)-N-(4-chloro-1-(tetrahydro-2H-pyran-2-yl)-1H-indazol-5-yl)-1,3,4-oxadiazol-2-amine ((130 mg, 316.41 μmol, 55.8% yield) as a red solid.

### Step 9: N-(3-(5-((4-chloro-1-(tetrahydro-2H-pyran-2-yl)-1H-indazol-5-yl)amino)-1,3,4-oxadiazol-2-yl)phenyl)-1-methyl-1H-pyrazole-4-carboxamide

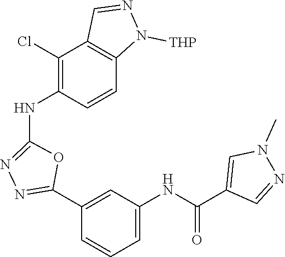

To a mixture of 5-(3-aminophenyl)-N-(4-chloro-1-(tetrahydro-2H-pyran-2-yl)-1H-indazol-5-yl)-1,3,4-oxadiazol-2-amine (70 mg, 170.38 μmol) in Py (2 mL) was added 1-methyl-1H˜pyrazole-4-carboxylic acid (21.5 mg, 170.38 μmol) and EDCI (81.7 mg, 425.94 μmol) at r.t., and then the mixture was stirred at r.t. for 4 hr. The reaction mixture was poured into water (20 mL) filtered by filter paper, the filter cake was washed by MeOH (10 mL). The filter layers was concentrated under reduced pressure to give N-(3-(5-((4-chloro-1-(tetrahydro-2H-pyran-2-yl)-1H-indazol-5-yl)amino)-1,3,4 oxadiazol-2-yl)phenyl)-1-methyl-1H-pyrazole-4-carboxamide (88.4 mg, crude) as a red solid.

### Example 55: N-(3-(5-((4-chloro-1H-indazol-5-yl)amino)-1,3,4-oxadiazol-2-yl)phenyl)-1-methyl-1H-pyrazole-4-carboxamide

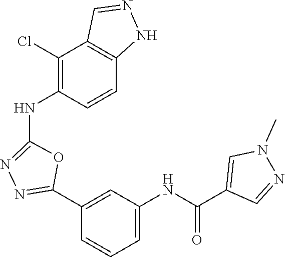 [SMILES: Cn1cc(C(=O)Nc2cccc(-c3nnc(Nc4ccc5[nH]ncc5c4Cl)o3)c2)cn1]

To a solution of N-(3-(5-((4-chloro-1-(tetrahydro-2H-pyran-2-yl)-1H-indazol-5-yl)amino)-1,3,4-oxadiazol-2-yl)phenyl)-1-methyl-1H-pyrazole-4-carboxamide (60 mg, 115.62 μmol,) was added HCl/dioxane (4 M, 12. mL) at r.t., and then mixture was stirred at r.t. for 2 hr. The reaction solution was concentrated under reduced pressure to give a crude product. The crude product was purified by preparative HPLC (30-80% MeCN in H₂O) to give N-(3-(5-((4-chloro-1H-indazol-5-yl)amino)-1,3,4-oxadiazol-2-yl)phenyl)-1-methyl-1H-pyrazole-4-carboxamide (21.1 mg, 38.06 μmol, 39.9% yield) as an off-white solid. LC-MS (ES⁺, Method A), 0.39 min, m/z 435.1 [M+H]⁺. ¹H NMR (400 MHz, DMSO-d₆) δ 13.46 (s, 1H) 10.08-10.04 (m, 2H) 8.34 (s, 2H) 8.14 (s, 1H) 8.04 (s, 1H) 7.89 (d, J=8.4 Hz, 1H) 7.77 (d, J=8.8 Hz, 1H) 7.60 (d, J=8.8 Hz, 1H) 7.56-7.51 (m, 2H) 3.90 (s, 3H).

### General Route or the Synthesis of Examples 56-60:

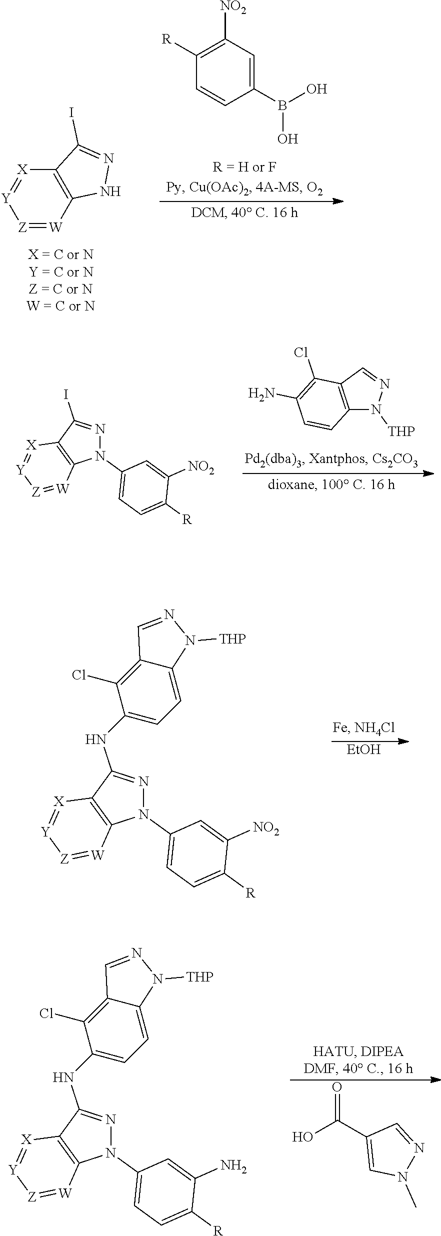

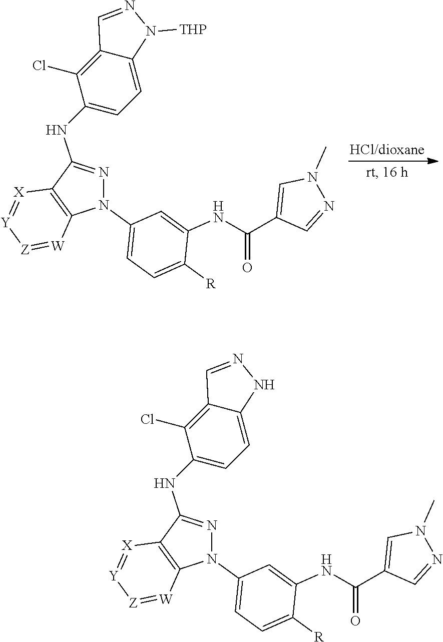

### Step 1: 3-iodo-1-(3-nitrophenyl)-1H-pyrazolo[4,3-b]pyridine

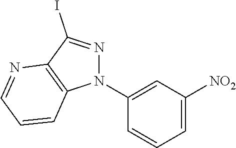 [SMILES: O=[N+]([O-])c1cccc(-n2nc(I)c3ncccc32)c1]

To a solution of 3-iodo-1H-pyrazolo[4,3-b]pyridine (2 g, 8.16 mmol, 1 eq) and (3-nitrophenyl)boronic acid (1.77 g, 10.61 mmol) in THF (20 mL) were added 4 Å MS (1 g) and Cu(OAc)₂ (2.22 g, 12.24 mmol), pyridine (1.29 g, 16.33 mmol, 1.32 mL) and boric acid (1.01 g, 16.33 mmol) at r.t. The mixture was stirred at r.t. for 16 hr under O₂ (15 Psi). The residue was diluted with H₂O (20 mL) and extracted with EtOAc 60 mL (3×20 mL). The combined organic layers were washed with brine (20 mL), dried over anhydrous Na₂SO₄, filtered and concentrated under reduced pressure to give a residue. The residue was purified by preparative HPLC (34-64% MeCN in H₂O) to afford 3-iodo-1-(3-nitrophenyl)pyrazolo[4,3-b]pyridine (420 mg, 1.15 mmol, 14.05% yield) as a yellow solid. LC-MS (ES⁺, Method A), 0.41 min, m/z 366.9 [M+H]⁺.

### Step 2: N-(4-chloro-1-(tetrahydro-2H-pyran-2-yl)-1H-indazol-5-yl)-1-(3-nitrophenyl)-1H-pyrazolo[4,3-b]pyridin-3-amine

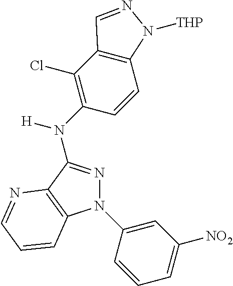

A mixture of 3-iodo-1-(3-nitrophenyl)pyrazolo[4,3-b]pyridine (200.00 mg, 546.28 umol), 4-chloro-1-tetrahydropyran-2-yl-indazol-5-amine (178.76 mg, 710.16 umol), Pd₂(dba)₃ (50.02 mg, 54.63 umol), Xantphos (63.22 mg, 109.26 umol) and Cs₂CO₃ (355.98 mg, 1.09 mmol) in dioxane (3 mL) was degassed and purged with N₂ for 3 times, and then the mixture was stirred at 90° C. for 16 hr under N₂ atmosphere. The reaction mixture was concentrated under reduced pressure to give a residue. The residue was purified by flash silica gel chromatography eluting with 5-25% EtOAc in Pet. Ether to give N-(4-chloro-1-tetrahydropyran-2-yl-indazol-5-yl)-1-(3-nitrophenyl)pyrazolo[4,3-b]pyridin-3-amine (50 mg, 102.06 umol, 18.68% yield) as a yellow solid. LC-MS (ES⁺, Method A), 0.60 min, m/z 490.1 [M+H]⁺.

### Step 3: 1-(3-aminophenyl)-N-(4-chloro-1-(tetrahydro-2H-pyran-2-yl)-1H-indazol-5-yl)-1H-pyrazolo[4,3-b]pyridin-3-amine

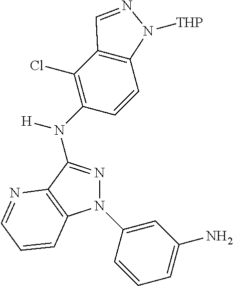

To a solution of N-(4-chloro-1-tetrahydropyran-2-yl-indazol-CI 5-yl)-1-(3-nitrophenyl)pyrazolo[4,3-b]pyridin-3-amine (80 mg, 163.29 umol) in EtOH (1 mL) and H₂O (0.1 mL) was added Fe (45.60 mg, 816.47 umol) and NH₄Cl (43.67 mg, 816.47 umol). The mixture was stirred at 50° C. for 16 hr. The reaction mixture was filtered to removed the insoluble Fe and the filter liquor was concentrated in vacuo to give a C₁ residue. The residue was diluted with H₂O (10 mL) and extracted with EtOAc (3×10 mL). The combined organic layers were washed with brine (10 mL), dried over anhydrous Na₂SO₄, filtered and concentrated under reduced pressure to give 1-(3-aminophenyl)-N-(4-chloro-1-tetrahydropyran-2-yl-indazol-5-yl)pyrazolo[4,3-b]pyridin-3-amine (65 mg, 141.33 umol, 86.55% yield) as a yellow oil, which was used directly in the next step without further purification. LC-MS (ES⁺, Method A), 0.57 min, m/z 460.3 [M+H]⁺.

General Method A for the synthesis of example 56:

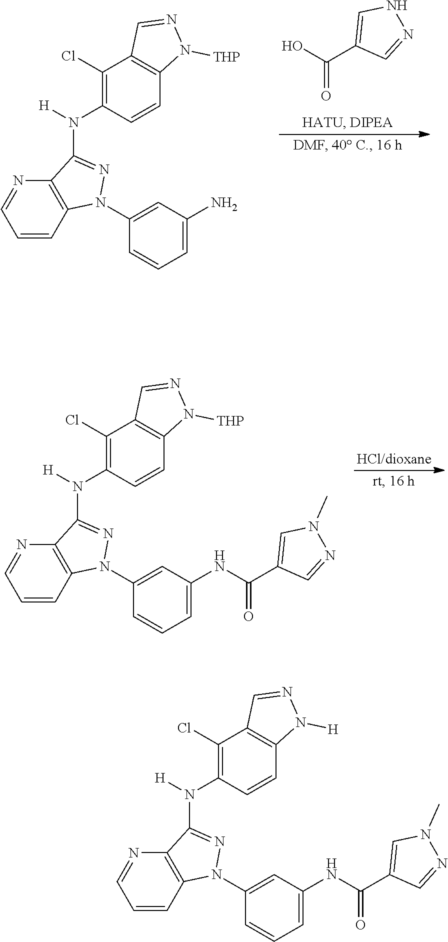

### Step 1: N-(3-(3-((4-chloro-1-(tetrahydro-2H-pyran-2-yl)-1H-indazol-5-yl)amino)-1H-pyrazolo[4,3-b]pyridin-1-yl)phenyl)-1-methyl-1H-pyrazole-4-carboxamide

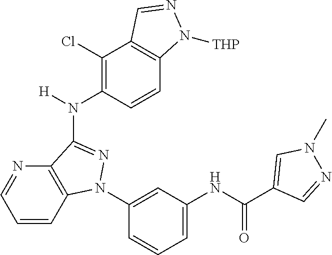

To a solution of 1-(3-aminophenyl)-N-(4-chloro-1-tetrahydropyran-2-yl-indazol-5-yl)pyrazolo[4,3-b]pyridin-3-amine (60.00 mg, 130.45 umol) and 1-methylpyrazole-4-carboxylic acid (32.90 mg, 260.91 umol) in DMF (0.5 mL) was added HATU (99.21 mg, 260.91 umol) and DIEA (84.30 mg, 652.27 umol, 113.61 uL). The mixture was stirred at 40° C. for 16 hr. The reaction mixture was poured into water (10 mL), and extracted with EtOAc (3×10 mL). The combined organic phase was washed with brine (10 mL), dried with anhydrous sodium sulfate, filtered and concentrated in vacuum. The residue was purified by preparative HPLC (55-85% MeCN in H₂O) to afford N-[3-[3-[(4-chloro-1-tetrahydropyran-2-yl-indazol-5-yl)amino]pyrazolo[4,3-b]pyridin-1-yl]phenyl]-1-methyl-pyrazole-4-carboxamide (50 mg, 88.02 umol, 67.47% yield) as a yellow solid. LC-MS (ES⁺, Method A), 0.5 min, m/z 568.2 [M+H]⁺.

### Example 56: N-(3-(3-((4-chloro-1H-indazol-5-yl)amino)-1H-pyrazolo[4,3-b]pyridin-1-yl)phenyl)-1-methyl-1H-pyrazole-4-carboxamide

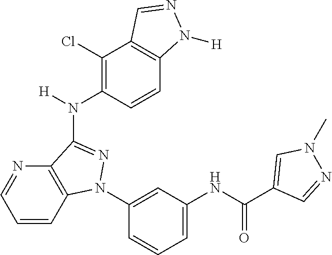 [SMILES: Cn1cc(C(=O)Nc2cccc(-n3nc(Nc4ccc5[nH]ncc5c4Cl)c4ncccc43)c2)cn1]

A mixture of N-[3-[3-[(4-chloro-1-tetrahydropyran-2-yl-indazol-5-yl)amino]pyrazolo[4,3-b]pyridin-1-yl]phenyl]-1-methyl-pyrazole-4-carboxamide (50 mg, 88.02 umol) in HCl/dioxane (4 M, 2 mL) was stirred at 25° C. for 16 hr. The reaction mixture was concentrated under reduced pressure. The residue was purified by preparative HPLC (34-64% MeCN in H₂O) to afford N-[3-[3-[(4-chloro-1H-indazol-5-yl)amino]pyrazolo[4,3-b]pyridin-1-yl]phenyl]-1-methyl-pyrazole-4-carboxamide (12.5 mg, 23.21 umol, 26.36% yield, FA salt) as a brown solid. LC-MS (ES⁺, Method A), 0.55 min, m/z 484.2 [M+H]⁺. ¹H NMR (400 MHz, DMSO-d₆) δ 13.35 (s, 1H), 10.03 (s, 1H), 8.59 (d, J=4.0 Hz, 1H), 8.38-8.32 (m, 2H), 8.28 (d, J=8.8 Hz, 1H), 8.22 (s, 1H), 8.14 (s, 1H), 8.10 (s, 1H), 8.05 (s, 1H), 7.64-7.60 (m, 3H), 7.52-7.48 (m, 2H), 3.91 (s, 3H).

General Method B for the synthesis of Example 57:

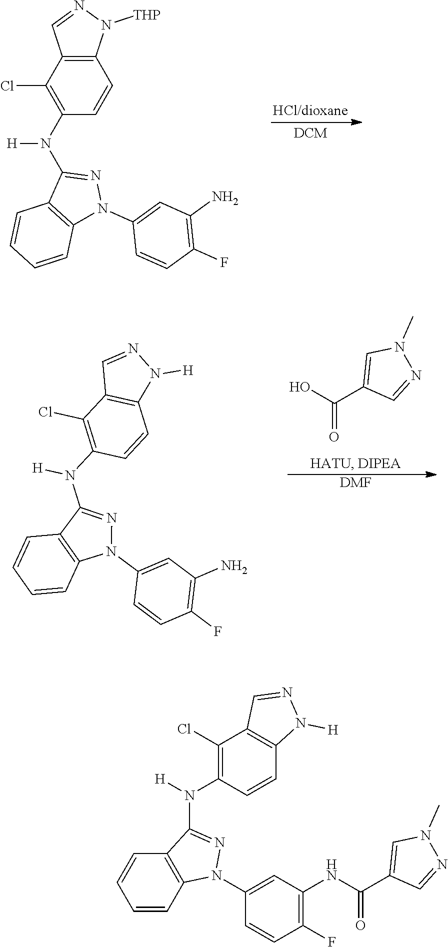

### Step 1: 1-(3-amino-4-fluorophenyl)-N-(4-chloro-1H-indazol-5-yl)-1H-indazol-3-amine

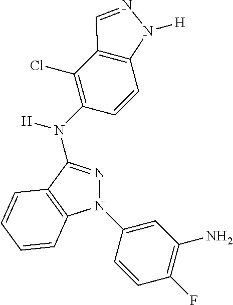 [SMILES: Nc1cc(-n2nc(Nc3ccc4[nH]ncc4c3Cl)c3ccccc32)ccc1F]

To a solution of 1-(3-amino-4-fluorophenyl)-N-(4-chloro-1-(tetrahydro-2H-pyran-2-yl)-1H-indazol-5-yl)-1H-indazol-3-amine (70 mg, 146.77 umol) in dioxane (0.5 mL) was added HCl/dioxane (4 M, 1 mL). The mixture was stirred at 20° C. for 1 hr. The reaction mixture was concentrated under reduced pressure to remove solvent to give 1-(3-amino-4-fluorophenyl)-N-(4-chloro-1H-indazol-5-yl)-1H-indazol-3-amine (200 mg, 509.14 umol) as a yellow solid. LC-MS (ES⁺, Method A), 0.54 min, m/z 393.1 [M+H]⁺.

### Example 57: N-(5-(3-((4-chloro-1H-indazol-5-yl)amino)-1H-indazol-1-yl)-2-fluorophenyl)-1-methyl-1H-pyrazole-4-carboxamide

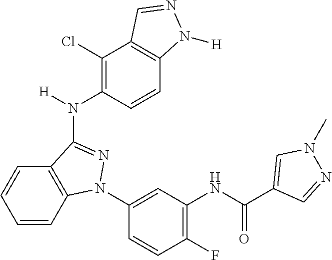 [SMILES: Cn1cc(C(=O)Nc2cc(-n3nc(Nc4ccc5[nH]ncc5c4Cl)c4ccccc43)ccc2F)cn1]

To a solution of 1-(3-amino-4-fluorophenyl)-N-(4-chloro-1H-indazol-5-yl)-1H-indazol-3-amine (200 mg, 509.14 umol), 1-methyl-1H-pyrazole-4-carboxylic acid (64.21 mg, 509.14 umol) in DMF (2 mL) was added HATU (387.18 mg, 1.02 mmol) and DIEA (197.41 mg, 1.53 mmol). The mixture was stirred at 25° C. for 16 hr. The reaction mixture was poured into water (30 mL), extracted with Ethyl acetate (2×20 mL). The combined organic layers were washed with brine (2×10 mL), dried over Na₂SO₄, filtered and concentrated. The residue was purified by prep-HPLC (30-60% MeCN in H₂O) to give N-(5-(3-((4-chloro-1H-indazol-5-yl)amino)-1H-indazol-1-yl)-2-fluorophenyl)-1-methyl-1H-pyrazole-4-carboxamide (4.1 mg, 7.78 umol, 1.5% yield) as a white solid. LC-MS (ES⁺, Method A), 0.62 min, m/z 501.1 [M+H]⁺. ¹H NMR (400 MHz, DMSO-d₆) δ 13.30 (s, 1H), 9.84 (s, 1H), 8.44 (s, 1H), 8.36 (s, 1H), 8.07 (s, 1H), 8.05-7.98 (m, 3H), 7.83 (dd, J=8.8, 15.7 Hz, 2H), 7.56-7.45 (m, 3H), 7.44-7.37 (m, 1H), 7.20 (t, J=7.6 Hz, 1H), 3.90 (s, 3H).

Compounds prepared in a similar manner to that set out above are given below in Table 15.

TABLE 15

| Example | Structure | Method | LCMS | ¹H NMR |
|---|---|---|---|---|
| 58 | 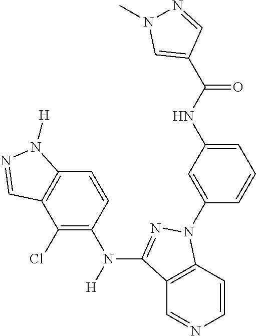 [SMILES: Cn1cc(C(=O)Nc2cccc(-n3nc(Nc4ccc5[nH]ncc5c4Cl)c4cnccc43)c2)cn1] | A | LC-MS (ES⁺, Method A), 0.39 min, m/z 484.2 [M + H]⁺ | ¹H NMR (400 MHz, DMSO-d₆) δ 10.20 (s, 1 H), 9.78 (s, 1 H), 9.62 (s, 1 H), 8.64 (d, J = 7.2 Hz, 1 H), 8.37 (s, 1 H), 8.21 (s, 1 H), 8.16- 8.10 (m, 2 H), 8.05 (s, 1 H), 7.89 (d, J = 8.8 Hz, 1 H), 7.79 (d, J = 8.80 Hz, 1 H), 7.61 (d, J = 8.8 Hz, 1 H), 7.55 (t, J = 8.0 Hz, 1 H), 7.43 (d, J = 7.6 Hz, 1 H), 3.89 (s, 3 H). |
| 59 | 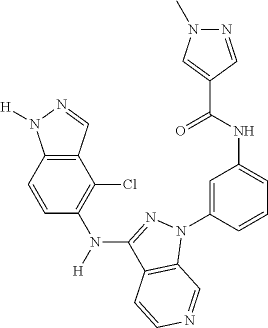 [SMILES: Cn1cc(C(=O)Nc2cccc(-n3nc(Nc4ccc5[nH]ncc5c4Cl)c4ccncc43)c2)cn1] | A | LC-MS (ES⁺, Method A), 0.42 min, m/z 484.1 [M + H]⁺ | ¹H NMR (400 MHz, MeOD) δ 9.29 (s, 1 H), 8.29 (s, 1 H), 8.25 (s, 1 H), 8.21 (s, 1 H), 8.10- 8.06 (m, 3 H), 7.60 (d, J = 7.2 Hz, 1 H), 7.60 (d, J = 7.2 Hz, 1 H), 7.55-7.51 (m, 3 H), 3.96 (s, 3 H). |
| 60 | 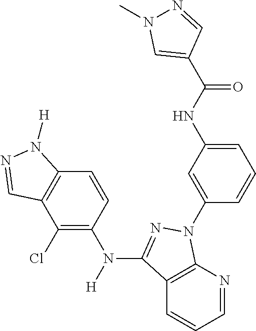 [SMILES: Cn1cc(C(=O)Nc2cccc(-n3nc(Nc4ccc5[nH]ncc5c4Cl)c4cccnc43)c2)cn1] | A | LC-MS (ES⁺, Method A), 0.55 min, m/z 484.2 [M + H]⁺ | ¹H NMR (400 MHz, DMSO-d₆) δ 9.99 (s, 1 H), 8.75 (s, 1 H), 8.65 (dd, J = 4.4, 1.2 Hz, 1 H), 8.40-8.50 (m, 2 H), 8.35 (s, 1 H), 8.11 (s, 1 H), 8.04 (s, 1 H), 7.99 (d, J = 8.8 Hz, 1 H), 7.93 (d, J = 8.8 Hz, 1 H), 7.66 (d, J = 8.8 Hz, 1 H), 7.59 (d, J = 8.8 Hz, 1 H), 7.42 (t, J = 8.4 Hz, 1 H), 7.29 (dd, J = 8.0, 4.4 Hz, 1 H), 3.90 (s, 3 H). |

General method for the synthesis of Example 61 and 62:

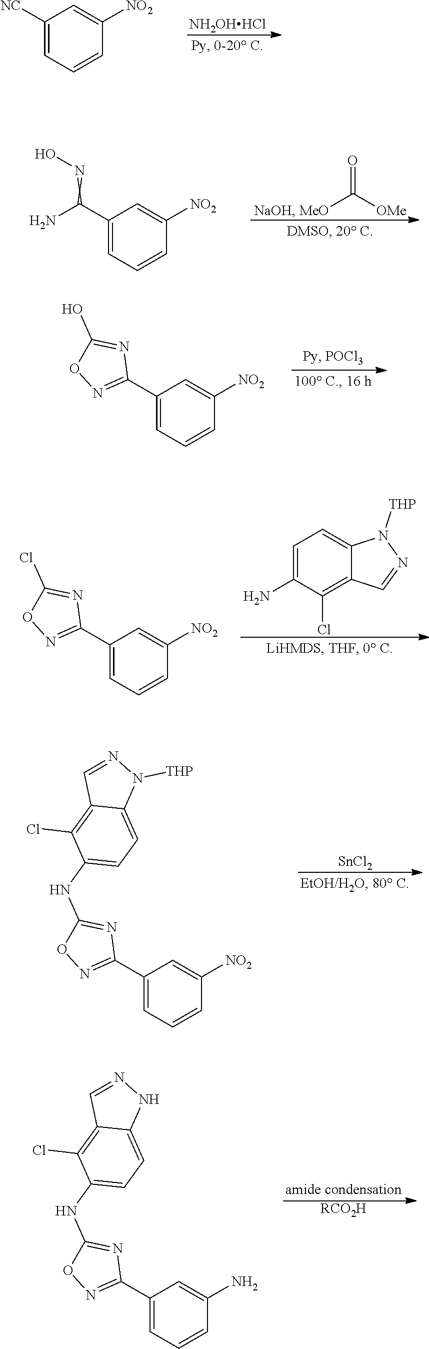

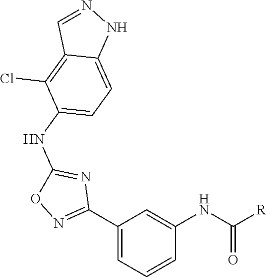

### Step 1: N′-hydroxy-3-nitrobenzimidamide

To a mixture of 3-nitrobenzonitrile (5 g, 33.76 mmol) in pyridine (60 mL) was added

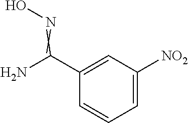 [SMILES: NC(=NO)c1cccc([N+](=O)[O-])c1]

NH₂OH·HCl (14.07 g, 202.54 mmol) at 0° C., then the mixture was stirred at 20° C. for 16 hr. The reaction mixture was diluted with water (200 mL) and then extracted with ethyl acetate (3×100 mL). The combined organic layers were washed with brine (2×50 mL), dried over anhydrous sodium sulfate, filtered and concentrated under reduced pressure to give N′-hydroxy-3-nitrobenzimidamide (5.5 g, 30.36 mmol, 89.94% yield) as a yellow solid.

### Step 2: 3-(3-nitrophenyl)-1,2,4-oxadiazol-5-ol

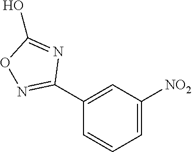 [SMILES: O=[N+]([O-])c1cccc(-c2noc(O)n2)c1]

To a mixture of N′-hydroxy-3-nitrobenzimidamide (5.5 g, 30.36 mmol) and dimethyl carbonate (4.10 g, 45.54 mmol, 3.83 mL) in DMSO (25 mL) was added NaOH (1.82 g, 45.54 mmol). The mixture was stirred at 20° C. for 2 hr. The mixture was diluted with H₂O (100 mL), HCl (1N) was added to adjust pH to 7, then filtered and washed with H₂O (300 mL) to give 3-(3-nitrophenyl)-1,2,4-oxadiazol-5-ol (3.4 g, 16.41 mmol, 54.06% yield) as a yellow solid.

### Step 3: 5-chloro-3-(3-nitrophenyl)-1,2,4-oxadiazole

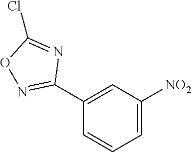 [SMILES: O=[N+]([O-])c1cccc(-c2noc(Cl)n2)c1]

To a solution of 3-(3-nitrophenyl)-1,2,4-oxadiazol-5-ol (500 mg, 2.41 mmol) in POCl₃ (10 mL) was added pyridine (286.40 mg, 3.62 mmol). The mixture was stirred at 100° C. for 16 hr. The reaction mixture was dropwisely into the ice water (200 mL), extracted with Ethyl acetate (2×80 mL). The combined organic layers were washed with brine (2×50 mL), dried over Na₂SO₄, filtered and concentrated under reduced pressure to give 5-chloro-3-(3-nitrophenyl)-1,2,4-oxadiazole (500 mg, 2.22 mmol, 91.82% yield) as a yellow solid.

### Step 4: N-(4-chloro-1-(tetrahydro-2H-pyran-2-yl)-1H-indazol-5-yl)-3-(3-nitrophenyl)-1,2,4-oxadiazol-5-amine

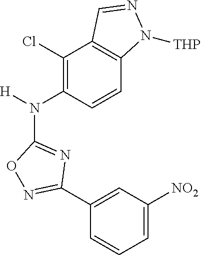

To A solution of 4-chloro-1-(tetrahydro-2H-pyran-2-yl)-1H-indazol-5-amine (111.58 mg, 443.29 umol) in THE (2 mL) was added LiHMDS (1 M, 886.57 uL) at 0° C. and stirred for 30 min, then the mixture was added 5-chloro-3-(3-nitrophenyl)-1,2,4-oxadiazole (100 mg, 443.29 umol) and stirred at 20° C. for 15.5 h. The reaction mixture was poured into the saturated NH₄Cl (80 mL), extracted with Ethyl acetate (2×30 mL). The combined organic layers were washed with brine (2×30 mL), dried over Na₂SO₄, filtered and concentrated under reduced pressure to give a residue. The residue was purified by column chromatography (SiO₂, Petroleum ether/Ethyl acetate=3:1) to give N-(4-chloro-1-(tetrahydro-2H-pyran-2-yl)-1H-indazol-5-yl)-3-(3-nitrophenyl)-1,2,4-oxadiazol-5-amine (195 mg, 442.34 umol, 99.79% yield) as a yellow oil. LC-MS (ES⁺, Method C), 0.586 min, m/z 441.1 [M+H]⁺.

### Step 5: 3-(3-aminophenyl)-N-(4-chloro-1H-indazol-5-yl)-1,2,4-oxadiazol-5-amine

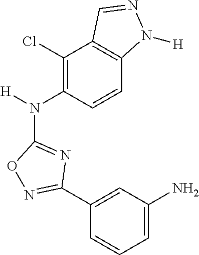 [SMILES: Nc1cccc(-c2noc(Nc3ccc4[nH]ncc4c3Cl)n2)c1]

To a solution of N-(4-chloro-1-(tetrahydro-2H-pyran-2-yl)-1H-indazol-5-yl)-3-(3-nitrophenyl)-1,2,4-oxadiazol-5-amine (195 mg, 442.34 umol) in EtOH (3 mL) and H₂O (0.1 mL) was added SnCl₂.2H₂O (501.62 mg, 2.22 mmol). The mixture was stirred at 80° C. for 2 hr. The residue was purified by prep-HPLC (column: Phenomenex luna C18 150*25 mm*10 um; mobile phase: [water(FA)-MeCN]; B %: 17%-47%, 10.5 min) to give 3-(3-aminophenyl)-N-(4-chloro-1H-indazol-5-yl)-1,2,4-oxadiazol-5-amine (75.1 mg, 218.35 umol, 49.36% yield) as a white solid. LC-MS (ES⁺, Method A), 0.343 min, m/z 227.0 [M+H]⁺.

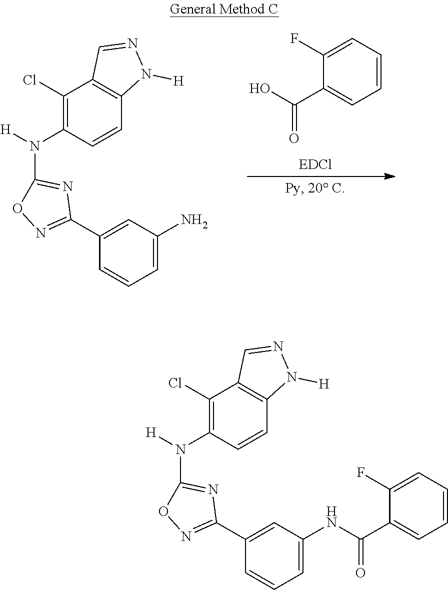 [SMILES: Nc1cccc(-c2noc(Nc3ccc4[nH]ncc4c3Cl)n2)c1 ; O=C(O)c1ccccc1F ; O=C(Nc1cccc(-c2noc(Nc3ccc4[nH]ncc4c3Cl)n2)c1)c1ccccc1F]

### Example 61: N-(3-(5-((4-chloro-1H-indazol-5-yl)amino)-1,2,4-oxadiazol-3-yl)phenyl)-2-fluorobenzamide

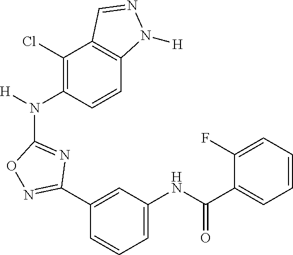 [SMILES: O=C(Nc1cccc(-c2noc(Nc3ccc4[nH]ncc4c3Cl)n2)c1)c1ccccc1F]

To a solution of 3-(3-aminophenyl)-N-(4-chloro-1H-indazol-5-yl)-1,2,4-oxadiazol-5-amine (100 mg, 306.05 umol), 2-fluorobenzoic acid (42.88 mg, 306.05 umol) in pyridine (2 mL) was added EDCI (146.68 mg, 765.13 umol). The mixture was stirred at 20° C. for 16 hr. The residue was purified by preparative HPLC (30-60% MeCN in H₂O) to give N-[3-[5-[(4-chloro-1H-indazol-5-yl)amino]-1,2,4-oxadiazol-3-yl]phenyl]-2-fluoro-benzamide (59.1 mg, 130.75 umol, 42.72% yield) as a white solid. LC-MS (ES⁺, Method A), 0.518 min, m/z 449.2 [M+H]⁺. ¹H NMR (400 MHz, DMSO-d₆) δ 13.54 (s, 1H), 10.61 (s, 1H), 8.32 (s, 1H), 8.18 (s, 1H), 7.89 (d, J=8.0 Hz, 1H), 7.70-7.55 (m, 6H), 7.58 (t, J=8.0, 15.6 Hz, 1H), 7.33 (dd, J=9.6, 17.6 Hz, 2H).

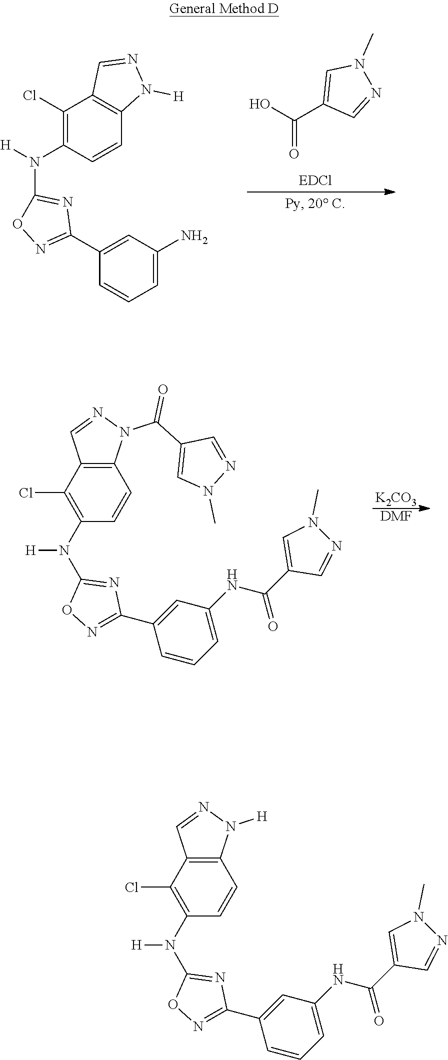

### Step 1: N-(3-(5-((4-chloro-1-(1-methyl-1H-pyrazole-4-carbonyl)-1H-indazol-5-yl)amino)-1,2,4-oxadiazol-3-yl)phenyl)-1-methyl-1H-pyrazole-4-carboxamide

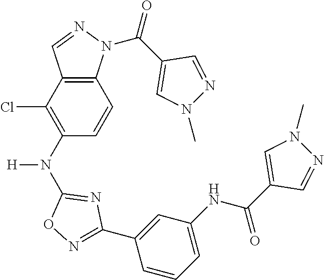 [SMILES: Cn1cc(C(=O)Nc2cccc(-c3noc(Nc4ccc5c(cnn5C(=O)c5cnn(C)c5)c4Cl)n3)c2)cn1]

To a solution of 3-(3-aminophenyl)-N-(4-chloro-1H-N indazol-5-yl)-1,2,4-oxadiazol-5-amine (70 mg, 214.24 umol), 1methyl-1H-pyrazole-4-carboxylic acid (27.02 mg, 214.24 umol) in pyridine (2 mL) was added EDCI (82.14 mg, 428.48 umol). The mixture was stirred at 20° C. for 16 hr. The reaction mixture was poured into water, a quantity of solid was collected by filtered to give N-(3-(5-((4-chloro-1-(1-methyl-1H-pyrazole-4-carbonyl)-1H-indazol-5-yl)amino)-1,2,4-oxadiazol-3-yl)phenyl)-1-methyl-1H-pyrazole-4-carboxamide (100 mg, crude) as a white solid.

### Example 62: N-(3-(5-((4-chloro-1H-indazol-5-yl)amino)-1,2,4-oxadiazol-3-yl)phenyl)-1-methyl-1H-pyrazole-4-carboxamide

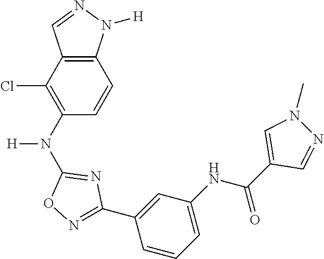 [SMILES: Cn1cc(C(=O)Nc2cccc(-c3noc(Nc4ccc5[nH]ncc5c4Cl)n3)c2)cn1]

To a solution of N-(3-(5-((4-chloro-1-(1-methyl-1H-pyrazole-4-carbonyl)-1H-indazol-5-yl)amino)-1,2,4-oxadiazol-3-yl)phenyl)-1-methyl-1H-pyrazole-4-carboxamide (100 mg, 184.18 umol) in DMF (2 mL) was added K₂CO₃ (76.37 mg, 552.55 umol). The mixture was stirred at 20° C. for 1 hr. The residue was purified by preparative HPLC (35-65% MeCN in H₂O) to give N-[3-[5-[(4-chloro-1H-indazol-5-yl)amino]1,2,4-oxadiazol-3-yl]phenyl]-1-methyl-pyrazole-4-carboxamide (6.7 mg, 13.78 umol, 7.48% yield, HCl salt) as an off-white solid. LC-MS (ES⁺, Method A), 0.461 min, m/z 435.2 [M+H]⁺. ¹H NMR (400 MHz, DMSO-d₆) δ 10.64 (s, 1H), 9.98 (s, 1H), 8.32 (s, 1H), 8.23-8.15 (m, 2H), 8.04-7.97 (m, 2H), 7.63 (s, 2H), 7.58 (d, J=7.6 Hz, 1H), 7.45 (t, J=8.0 Hz, 1H), 3.88 (s, 3H).

General method for the synthesis of Example 63 and 64:

### Step 1: 3-nitrobenzoyl isothiocyanate

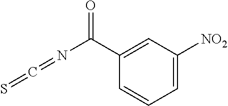 [SMILES: O=C(N=C=S)c1cccc([N+](=O)[O-])c1]

To a mixture of 3-nitrobenzoyl chloride (2 g, 10.78 mmol) in ACN (40 mL) was added

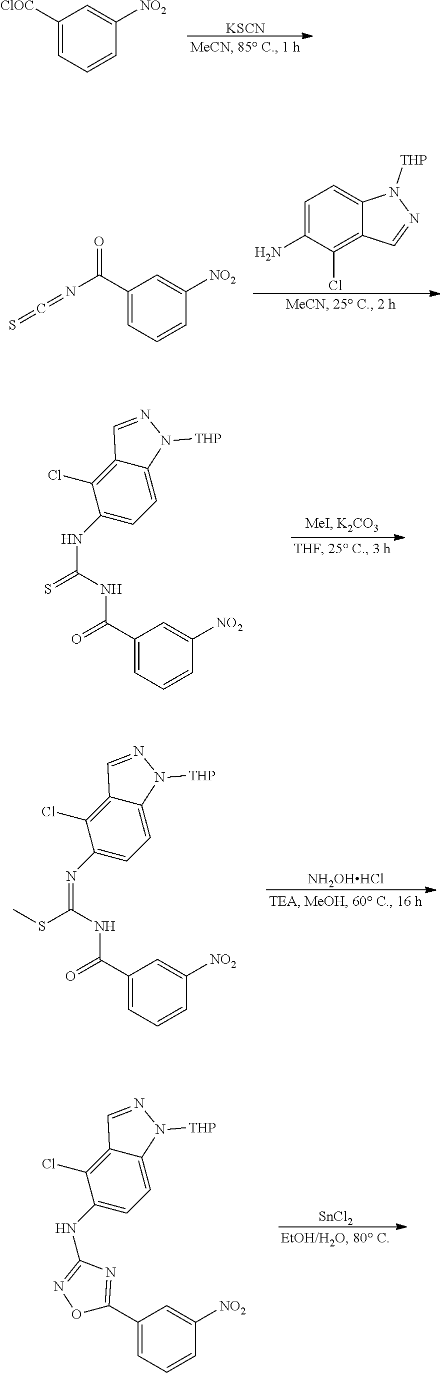

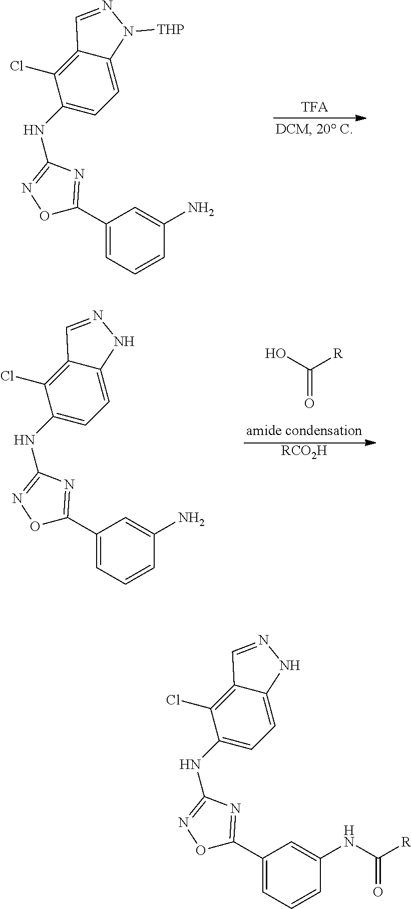

potassium thiocyanate (1.05 g, 10.78 mmol, 1.05 mL). The mixture was stirred at 85° C. for 1 hr. The reaction mixture was filtered to give a filter head and a filtrate and the filtrate was concentrated under vacuum to give 3-nitrobenzoyl isothiocyanate (2.2 g, 10.57 mmol, 98.04% yield) as a white solid.

### Step 2: N-((4-chloro-1-(tetrahydro-2H-pyran-2-yl)-1H-indazol-5-yl)carbamothioyl)-3-nitrobenzamide

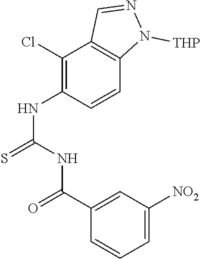

To a mixture of 3-nitrobenzoyl isothiocyanate (488.00 mg, 2.34 mmol) and 4-chloro-1-tetrahydropyran-2-yl-indazol-5-amine (590 mg, 2.34 mmol) in MeCN (5 mL). The mixture was stirred at 25° C. for 2 hr. The reaction mixture was diluted with H₂O (30 mL) and extracted with EtOAc (3×30 mL). The combined organic layers were washed with brine (50 mL), dried over Na₂SO₄, filtered and concentrated under reduced pressure to give a residue. The residue was added EtOAc (5 mL) and stirred at 20° C. for 10 min, the mixture was filtered and concentrated under vacuum to give N-((4-chloro-1-(tetrahydro-2H-pyran-2-yl)-1H-indazol-5-yl)carbamothioyl)-3-nitrobenzamide (1.1 g, crude) as a yellow solid.

### Step 3: (E)-methyl N′-(4-chloro-1-(tetrahydro-2H-pyran-2-yl)-1H-indazol-5-yl)-N-(3-nitrobenzoyl) carbamimidothioate

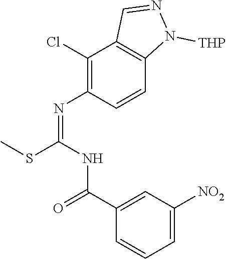

To a mixture of N-((4-chloro-1-(tetrahydro-2H-pyran-2-yl)-1H-indazol-5-yl)carbamothioyl)-3-nitrobenzamide (1.1 g, 2.39 mmol) in THE (15 mL) was added K₂CO₃ (661.12 mg, 4.78 mmol) and MeI (1.70 g, 11.96 mmol, 744.49 uL). The mixture was stirred at 20° C. for 3 hr. To the mixture was added H₂O (15 mL) and EtOAc (10 mL) and the mixture was stirred at 20° C. for 1 hr. the resulting mixture was filtered and the filter cake was concentrated under vacuum to give (E)-methyl N′-(4-chloro-1-(tetrahydro-2H-pyran-2-yl)-1H-indazol-5-yl)-N-(3-nitrobenzoyl)carbamimidothioate (800 mg, 1.69 mmol, 70.57% yield) as a yellow solid.

### Step 3: N-(4-chloro-1-(tetrahydro-2H-pyran-2-yl)-1H-indazol-5-yl)-5-(3-nitrophenyl)-1,2,4-oxadiazol-3-amine

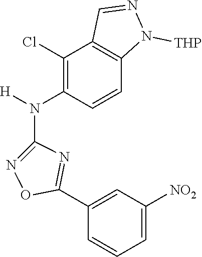

To a mixture of (E)-methyl N′-(4-chloro-1-(tetrahydro-2H-pyran-2-yl)-1H-indazol-5-yl)-N-(3-nitrobenzoyl)carbamimidothioate (660 mg, 1.39 mmol) in MeOH (6.6 mL) was added NH₂OH·HCl (145.16 mg, 2.09 mmol) and TEA (422.75 mg, 4.18 mmol, 581.50 uL), and the mixture was stirred at 60° C. for 10 hr. The reaction mixture was filtered and the filter cake was washed with water (2 mL) and dried under reduced pressure to give N-(4-chloro-1-(tetrahydro-2H-pyran-2-yl)-1H-indazol-5-yl)-5-(3-nitrophenyl)-1,2,4-oxadiazol-3-amine (290 mg, 657.84 umol, 47.24% yield) as a yellow solid.

### Step 4: 5-(3-aminophenyl)-N-(4-chloro-1-(tetrahydro-2H-pyran-2-yl)-1H-indazol-5-yl)-1,2,4-oxadiazol-3-amine

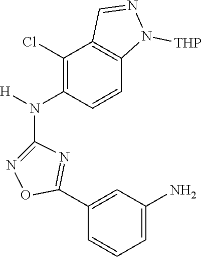

To a solution of N-(4-chloro-1-(tetrahydro-2H-pyran-2-yl)-1H-indazol-5-yl)-5-(3-nitrophenyl)-1,2,4-oxadiazol-3-amine (240 mg, 544.42 umol) in EtOH (2.5 mL) and H₂O (0.25 mL) was added SnCl₂.2H₂O (614.23 mg, 2.72 mmol) at 20° C. and then the mixture was stirred at 80° C. for 2 hr. The mixture was dissolved in water (20 mL) and extracted with EtOAc (3×50 mL). The combined organic layer was washed with saturated NaCl (2×20 mL), dried over Na₂SO₄, filtered and concentrated under reduced pressure to give 5-(3-aminophenyl)-N-(4-chloro-1-(tetrahydro-2H-pyran-2-yl)-1H-indazol-5-yl)-1,2,4-oxadiazol-3-amine (180 mg, crude) as a brown oil.

### Step 5: 5-(3-aminophenyl)-N-(4-chloro-1H-indazol-5-yl)-1,2,4-oxadiazol-3-amine

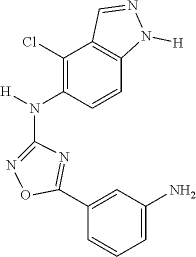 [SMILES: Nc1cccc(-c2nc(Nc3ccc4[nH]ncc4c3Cl)no2)c1]

To a solution of 5-(3-aminophenyl)-N-(4-chloro-1-(tetrahydro-2H-pyran-2-yl)-1H-indazol-5-yl)-1,2,4-oxadiazol-3-amine (180 mg, 438.11 umol) N in DCM (2 mL) was added TFA (3.96 g, 34.73 mmol, 2.57 mL) at 20° C. and then the mixture was stirred at 20° C. for 1 hr. The mixture was concentrated under reduced pressure to give 5-(3-aminophenyl)-N-(4-chloro-1H-indazol-5-yl)-1,2,4-oxadiazol-3-amine (100 mg, 306.05 umol, 69.86% yield) as a brown solid.

### Example 63: N-(3-(3-((4-chloro-1H-indazol-5-yl)amino)-1,2,4-oxadiazol-5-yl)phenyl)-1-methyl-1H-pyrazole-4-carboxamide

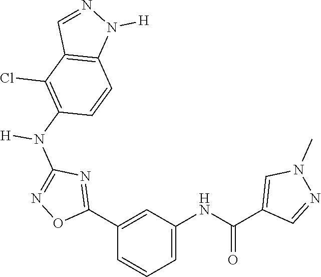 [SMILES: Cn1cc(C(=O)Nc2cccc(-c3nc(Nc4ccc5[nH]ncc5c4Cl)no3)c2)cn1]

To a solution of 5-(3-aminophenyl)-N-(4-chloro-1H-indazol-5-yl)-1,2,4-oxadiazol-3-amine (60 mg, 183.63 umol) in pyridine (2 mL) was added 1-methylpyrazole-4-carboxylic acid (27.79 mg, 220.36 umol) and EDCI (70.41 mg, 367.26 umol) at 20° C. and then the mixture was stirred at 20° C. for 16 hr. The mixture was concentrated under reduced pressure to give a residue. The residue was purified by preparative HPLC (28-58% MeCN in H₂O) and preparative HPLC (22-52% MeCN in H₂O) to give N-(3-(3-((4-chloro-1H-indazol-5-yl)amino)-1,2,4-oxadiazol-5-yl)phenyl)-1-methyl-1H-pyrazole-4-carboxamide (10.2 mg, 22.84 umol, 12.44% yield) as a light off-white solid. LC-MS (ES⁺, Method A), 0.474 min, m/z 435.1 [M+H]⁺. ¹H NMR (400 MHz, DMSO-d₆) δ 13.41 (s, 1H), 10.10 (s, 1H), 9.20 (s, 1H), 8.50 (t, J=1.6 Hz, 1H), 8.35 (s, 1H), 8.12 (s, 1H), 8.06-8.00 (m, 2H), 7.74 (d, J=8.0 Hz, 1H), 7.67-7.63 (m, 1H), 7.61-7.54 (m, 2H), 3.91 (s, 3H).

Compounds prepared in a similar manner to that set out above are given below in Table 16:

TABLE 16

| Example | Structure | LCMS | ¹H NMR |
|---|---|---|---|
| 64 | 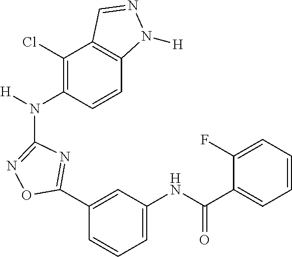 [SMILES: O=C(Nc1cccc(-c2nc(Nc3ccc4[nH]ncc4c3Cl)no2)c1)c1ccccc1F] | LC-MS (ES⁺, Method A), 0.543 min, m/z 449.1 [M + H]⁺. | ¹H NMR (400 MHz, DMSO- d₆) δ 13.40 (s, 1H), 10.74 (s, 1H), 9.23 (s, 1H), 8.57 (s, 1H), 8.11 (s, 1H), 7.95 (d, J = 8.0 Hz, 1H), 7.79 (d, J = 8.0 Hz, 1H), 7.73-7.69 (m, 1H), 7.67-7.55 (m, 4H), 7.40-7.32 (m, 2H) |

General route for the synthesis of Intermediate 6:

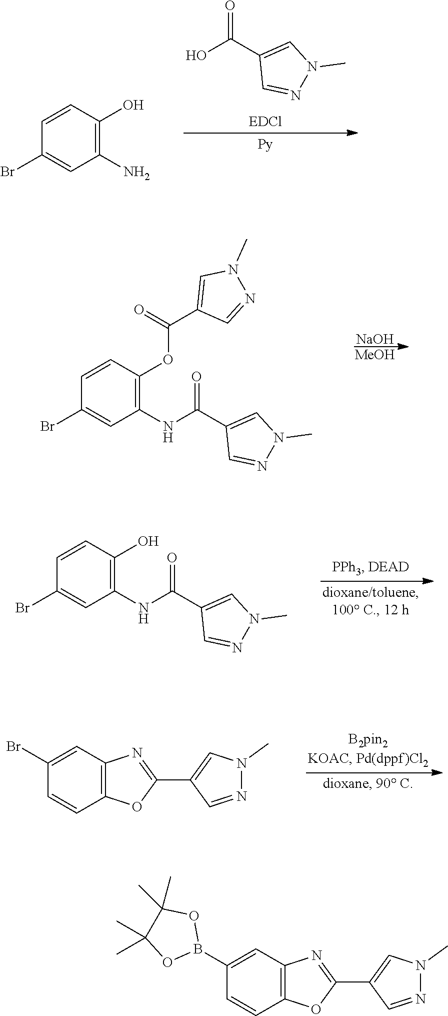

### Step 1: 4-bromo-2-(1-methyl-1H-pyrazole-4-carboxamido)phenyl 1-methyl-1H-pyrazole-4-carboxylate

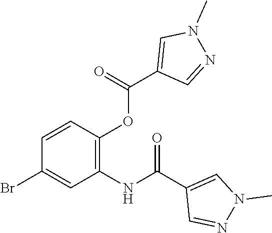 [SMILES: Cn1cc(C(=O)Nc2cc(Br)ccc2OC(=O)c2cnn(C)c2)cn1]

To a solution of 2-amino-4-bromo-phenol (10 g, 53.19 mmol), 1-methylpyrazole-4-carboxylic acid (10.06 g, 79.78 mmol) in pyridine (100 mL) was added EDCI (22.43 g, 117.01 mmol). The mixture was stirred at 40° C. for 16 hr. The reaction mixture was concentrated under reduced pressure to remove solvent. The residue was diluted with water (300 mL) and extracted with EtOAc (3×200 mL). The combined organic layers were washed with brine (2×100 mL), dried over Na₂SO₄, filtered and concentrated under reduced pressure to give 4-bromo-2-(1-methyl-1H-pyrazole-4-carboxamido)phenyl 1-methyl-1H-pyrazole-4-carboxylate (20 g, 49.48 mmol, 93.03% yield) as a brown solid. LC-MS (ES⁺, Method A), 0.402 min, m/z 405.9 [M+H]⁺.

### Step 2: N-(5-bromo-2-hydroxyphenyl)-1-methyl-1H-pyrazole-4-carboxamide

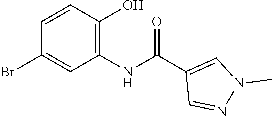 [SMILES: Cn1cc(C(=O)Nc2cc(Br)ccc2O)cn1]

To a solution of 4-bromo-2-(1-methyl-1H-pyrazole-4-carboxamido)phenyl 1-methyl-1H-pyrazole-4-carboxylate (20 g, 49.48 mmol) in MeOH (200 mL) and H₂O (20 mL) was added NaOH (3.96 g, 98.96 mmol). The mixture was stirred at 30° C. for 16 hr. The reaction mixture was concentrated under reduced pressure to remove solvent. The residue was diluted with water (300 mL) and adjusted the pH to 8 with HCl (1N), extracted with EtOAc (3×100 mL). The combined organic layers were washed with brine (2×200 mL), dried over Na₂SO₄, filtered and concentrated under reduced pressure to give N-(5-bromo-2-hydroxyphenyl)-1-methyl-1H-pyrazole-4-carboxamide (10 g, 33.77 mmol, 68.25% yield) as a brown solid. LC-MS (ES⁺, Method A), 0.393 min, m/z 298.0 [M+H]⁺.

### Step 3: 5-bromo-2-(1-methyl-1H-pyrazol-4-yl)benzo[d]oxazole

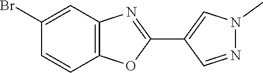 [SMILES: Cn1cc(-c2nc3cc(Br)ccc3o2)cn1]

To a mixture of N-(5-bromo-2-hydroxyphenyl)-1-methyl-1H-pyrazole-4-carboxamide (2 g, 6.75 mmol), PPh₃ (1.77 g, 6.75 mmol) in dioxane (20 mL) was dropwisely added a solution of DEAD (1.18 g, 6.75 mmol) in toluene (1 mL). The mixture was stirred at 100° C. for 16 hr. The reaction mixture was concentrated under reduced pressure to remove solvent. The residue was diluted with water (100 mL) and extracted with EtOAc (3×30 mL). The combined organic layers were washed with brine (2×50 mL), dried over Na₂SO₄, filtered and concentrated under reduced pressure to give a residue. The residue was purified by flash silica gel chromatography eluting with 0-60% EtOAc in Pet. Ether to give 5-bromo-2-(1-methyl-1H-pyrazol-4-yl)benzo[d]oxazole (1.88 g, 6.76 mmol, 100.00% yield) as a pink solid.

### Intermediate 6: 2-(1-methyl-1H-pyrazol-4-yl)-5-(4,4,5,5-tetramethyl-1,3,2-dioxaborolan-2-yl)benzo[d]oxazole

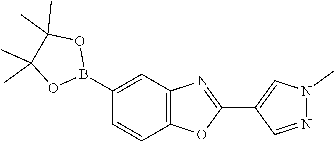

To a mixture of 5-bromo-2-(1-methyl-1H-pyrazol-4-yl)benzo[d]oxazole (1.88 g, 6.76 mmol), 4,4,5,5-tetramethyl-2-N (4,4,5,5-tetramethyl-1,3,2-dioxaborolan-2-yl)-1,3,2-dioxaborolane (1.89 g, 7.44 mmol), KOAc (1.33 g, 13.52 mmol), Pd(dppf)Cl₂ (494.64 mg, 676.01 umol) in dioxane (20 mL) was degassed and purged with nitrogen for 3 times, and then the mixture was stirred at 90° C. for 16 hr. The reaction mixture was poured into water (100 mL), extracted with Ethyl acetate (2×50 mL). The combined organic layers were washed with brine (2×50 mL), dried over Na₂SO₄, filtered and concentrated. The residue was purified by column chromatography (SiO₂, Petroleum ether/Ethyl acetate=3:1) to give 2-(1-methyl-1H-pyrazol-4-yl)-5-(4,4,5,5-tetramethyl-1,3,2-dioxaborolan-2-yl)benzo[d]oxazole (1.8 g, 5.54 mmol, 81.89% yield) as a white solid.

General method for the synthesis of Example 65 and 66:

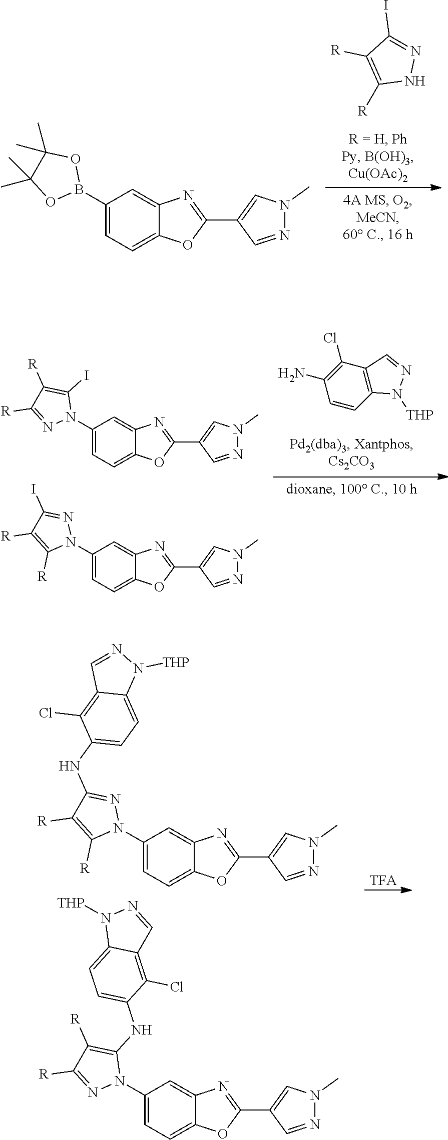

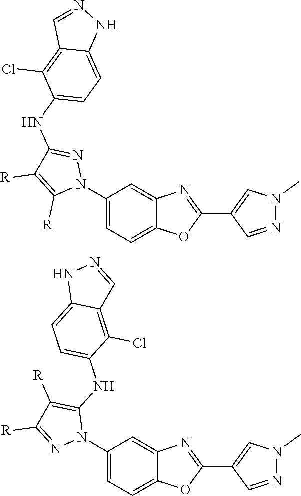

### Step 1: 5-(3-iodo-1H-pyrazol-1-yl)-2-(1-methyl-1H-pyrazol-4-yl)benzo[d]oxazole

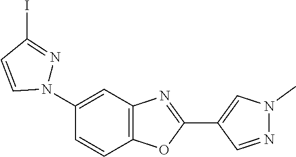 [SMILES: Cn1cc(-c2nc3cc(-n4ccc(I)n4)ccc3o2)cn1]

To a solution of 2-(1-methylpyrazol-4-yl)-5-(4,4,5,5-tetramethyl-1,3,2-dioxaborolan-2-yl)-1,3-benzoxazole (1.8 g, 5.54 mmol), 3-iodo-1H-pyrazole (1.07 g, 5.54 mmol) in MeCN (20 mL) was added pyridine (875.73 mg, 11.07 mmol), boric acid (684.53 mg, 11.07 mmol) and Cu(OAc)₂ (1.51 g, 8.30 mmol). The mixture was stirred at 60° C. for 12 hr. The reaction mixture was poured into water (30 mL), extracted with Ethyl acetate (2×20 mL). The combined organic layers were washed with brine (2×10 mL), dried over Na₂SO₄, filtered and concentrated. The residue was purified by preparative HPLC (27-57% MeCN in H₂O) to give 5-(3-iodo-1H-pyrazol-1-yl)-2-(1-methyl-1H-pyrazol-4-yl)benzo[d]oxazole (290 mg, 741.37 umol, 13.39% yield) as a white solid.

### Step 2: 4-chloro-N-(1-(2-(1-methyl-1H-pyrazol-4-yl)benzo[d]oxazol-5-yl)-1H-pyrazol-3-yl)-1-(tetrahydro-2H-pyran-2-yl)-1H-indazol-5-amine

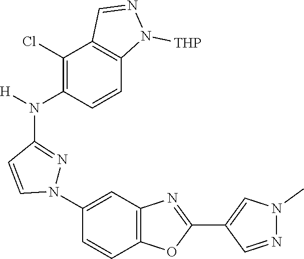

To a mixture of 5-(3-iodo-1H-pyrazol-1-yl)-2-(1-methyl-1H-pyrazol-4-yl)benzo[d]oxazole (290 mg, 741.37 umol), 4-chloro-1-tetrahydropyran-2-yl-indazol-5-amine (186.61 mg, 741.37 umol), Pd₂(dba)₃ (67.89 mg, 74.14 umol), Xantphos (85.79 mg, 148.27 umol) and Cs₂CO₃ (483.11 mg, 1.48 mmol) in dioxane (3 mL) was degassed and purged with nitrogen for 3 times, and then the mixture was stirred at 100° C. for 12 hr under nitrogen atmosphere. The reaction mixture was poured into water (50 mL), extracted with Ethyl acetate (2×30 mL). The combined organic layers were washed with brine (2×30 mL), dried over Na₂SO₄, filtered and concentrated. The residue was purified by column chromatography (SiO₂, Petroleum ether/Ethyl acetate=2:1) to give 4-chloro-N-(1-(2-(1-methyl-1H-pyrazol-4-yl)benzo[d]oxazol-5-yl)-1H-pyrazol-3-yl)-1-(tetrahydro-2H-pyran-2-yl)-1H-indazol-5-amine (185 mg, 359.25 umol, 48.46% yield) as a yellow oil. LC-MS (ES⁺, Method A), 0.598 min, m/z 515.3 [M+H]⁺.

### Example 65: 4-chloro-N-(1-(2-(1-methyl-1H-pyrazol-4-yl)benzo[d]oxazol-5-yl)-1H-pyrazol-3-yl)-1H-indazol-5-amine

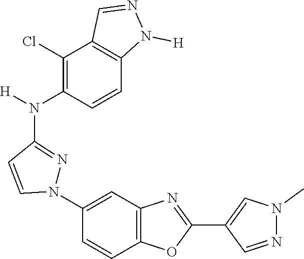 [SMILES: Cn1cc(-c2nc3cc(-n4ccc(Nc5ccc6[nH]ncc6c5Cl)n4)ccc3o2)cn1]

To a solution of 4-chloro-N-(1-(2-(1-methyl-1H-pyrazol-4-yl)benzo[d]oxazol-5-yl)-1H-pyrazol-3-yl)-1-(tetrahydro-2H-pyran-2-yl)-1H-indazol-5-amine (180 mg, 349.54 umol) in DCM (1 mL) was added TFA (1.54 g, 13.51 mmol). The mixture was stirred at 20° C. for 2 hr. The reaction mixture was concentrated under reduced pressure to remove solvent. The residue was purified by preparative HPLC (25-55% MeCN in H₂O) to give 4-chloro-N-(1-(2-(1-methyl-1H-pyrazol-4-yl)benzo[d]oxazol-5-yl)-1H-pyrazol-3-yl)-1H-indazol-5-amine (39 mg, 89.16 umol, 25.51% yield) as an off-white solid LC-MS (ES⁺, Method A), 0.513 min, m/z 431.3 [M+H]⁺. ¹H NMR (400 MHz, DMSO-d₆) δ 13.22 (s, 1H), 8.58 (s, 1H), 8.41 (d, J=2.4 Hz, 1H), 8.24 (d, J=9.2 Hz, 1H), 8.13 (s, 1H), 8.05 (s, 2H), 8.00 (s, 1H), 7.81-7.74 (m, 2H), 7.55 (d, J=9.2 Hz, 1H), 6.25 (d, J=2.4 Hz, 1H), 3.97 (s, 3H).

Compounds prepared in a similar manner to that set out above are given below in Table 17.

TABLE 17

| Example | Structure | LCMS | ¹H NMR |
|---|---|---|---|
| 66 | 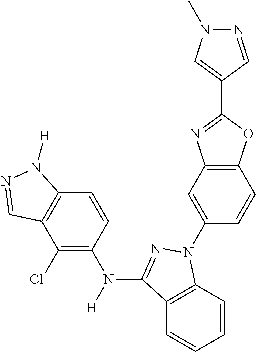 [SMILES: Cn1cc(-c2nc3cc(-n4nc(Nc5ccc6[nH]ncc6c5Cl)c5ccccc54)ccc3o2)cn1] | LC-MS (ES⁺, Method A), min, m/z 482.9. [M + H]⁺. | ¹H NMR (400 MHz, DMSO-d₆) δ 13.32 (s, 1H), 8.59 (s, 1H), 8.44 (s, 1H), 8.14 (s, 1H), 8.08 (s, 1H), 8.02 (d, J = 8.0 Hz, 1H), 7.95-7.89 (m, 2H), 7.81 (t, J = 8.8 Hz, 2H), 7.66 (dd, J = 2.0, 8.8 Hz, 1H), 7.55 (d, J = 8.8 Hz, 1H), 7.49 (t, J = 7.6 Hz, 1H), 7.20 (t, J = 7.6 Hz, 1H), 3.97 (s, 3H). |

General method for the synthesis of Example 67:

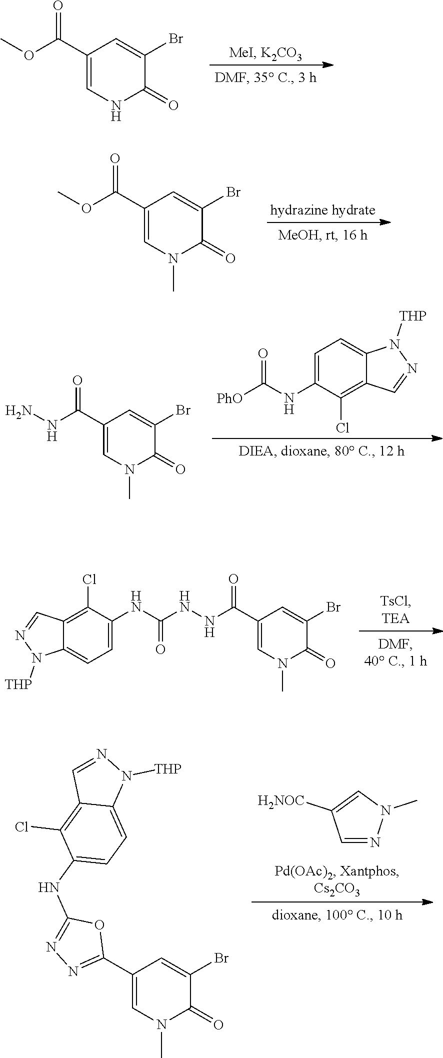

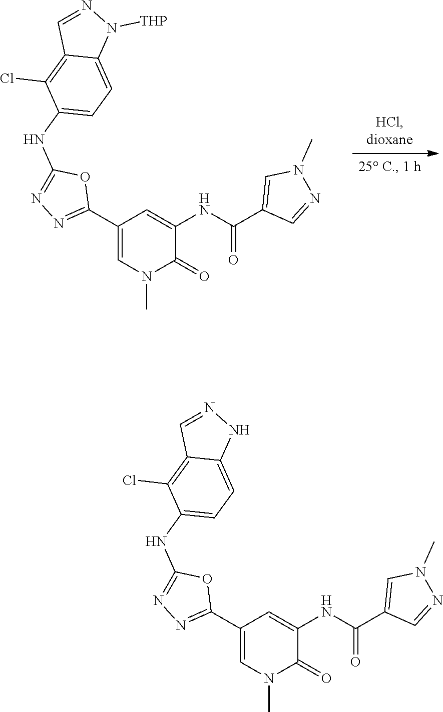

### Step 1: 3-iodo-1-(3-nitrophenyl)pyrazolo[4,3-c]pyridine

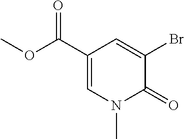

To a mixture of methyl 5-bromo-6-oxo-1H-pyridine-3-carboxylate (8.8 g, 37.93 mmol) and K₂CO₃ (10.48 g, 75.85 mmol) in DMF (50 mL) was added Mel (8.07 g, 56.89 mmol, 3.54 mL). The mixture was stirred at 35° C. for 3 hr. The reaction mixture was diluted with H₂O (70 mL) and extracted with ethyl acetate (3×50 mL). The combined organic layers were washed with brine (100 mL), dried over anhydrous Na₂SO₄, filtered and concentrated under reduced pressure to methyl 5-bromo-1-methyl-6-oxo-pyridine-3-carboxylate (8.7 g, 35.36 mmol, 93.23% yield) as a brown solid. LC-MS (ES⁺, Method A), 0.47 min, m/z 207.0 [M+H]⁺.

### Step 2: 5-bromo-1-methyl-6-oxo-pyridine-3-carbohydrazide

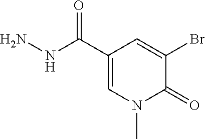

To a solution of methyl 5-bromo-1-methyl-6-oxo-pyridine-3-carboxylate (2 g, 8.13 mmol, 1 eq) in MeOH (20 mL) was added hydrazine hydrate (4.13 g, 82.50 mmol, 4.01 mL, 10.15 eq). The mixture was stirred at 70° C. for 4 hr. The reaction mixture was concentrated under reduced pressure to give a residue. The residue was triturated with EtOH (10 mL) at 25° C. for 30 min and filtered to give 5-bromo-1-methyl-6-oxo-pyridine-3-carbohydrazide (1 g, 4.06 mmol, 50.00% yield) as an off-white solid. LC-MS (ES⁺, Method A), 0.26 min, m/z 248.0 [M+H]⁺.

### Step 3: 1-[(5-bromo-1-methyl-6-oxo-pyridine-3-carbonyl)amino]-3-(4-chloro-1-tetrahydropyran-2-yl-indazol-5-yl)urea

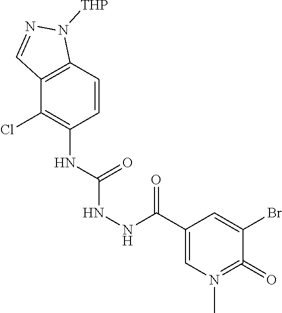

To a solution of 5-bromo-1-methyl-6-oxo-pyridine-3-carbohydrazide (86.03 mg, 349.63 umol, 1 eq) and phenyl N-(4-chloro-1-tetrahydropyran-2-yl-indazol-5-yl)carbamate (130 mg, 349.63 umol) in dioxane (2 mL) was added DIEA (135.56 mg, 1.05 mmol, 182.70 uL). The mixture was stirred at 80° C. for 16 hr. The reaction mixture was concentrated under reduced pressure to give a residue. The residue was triturated with EtOAc (3 mL) at 25° C. for 30 min and filtered to give 1-[(5-bromo-1-methyl-6-oxo-pyridine-3-carbonyl)amino]-3-(4-chloro-1-tetrahydropyran-2-yl-indazol-5-yl)urea (120 mg, 229.11 umol, 65.53% yield) as a white solid. LC-MS (ES⁺, Method A), 0.43 min, m/z 441.0 [M+H]⁺.

### Step 4: 3-bromo-5-[5-[(4-chloro-1-tetrahydropyran-2-yl-indazol-5-yl)amino]-1,3,4-oxadiazol-2-yl]-1-methyl-pyridin-2-one

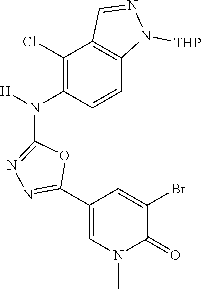

To a solution of 1-[(5-bromo-1-methyl-6-oxo-pyridine-3-carbonyl)amino]-3-(4-chloro-1-tetrahydropyran-2-yl-indazol-5-yl)urea (200 mg, 381.85 umol) in DMF (3 mL) was added TosCl (182.00 mg, 954.62 umol) and TEA (115.92 mg, 1.15 mmol, 159.45 uL). The mixture was stirred at 40° C. for 2 hr. The reaction mixture was a concentrated under reduced pressure to give a residue. The residue was purified by preparative HPLC (35-65% MeCN in H₂O) to give 3-bromo-5-[5-[(4-chloro-1-tetrahydropyran-2-yl-indazol-5-yl)amino]-1,3,4-oxadiazol-2-yl]-1-methyl-pyridin-2-one (70 mg, 138.41 umol, 36.25% yield) as a pink solid. LC-MS (ES⁺, Method A), 0.66 min, m/z 505.0 [M+H]⁺.

### Step 5: N-[5-[5-[(4-chloro-1-tetrahydropyran-2-yl-indazol-5-yl)amino]-1,3,4-oxadiazol-2-yl]-1-methyl-2-oxo-3-pyridyl]-1-methyl-pyrazole-4-carboxamide

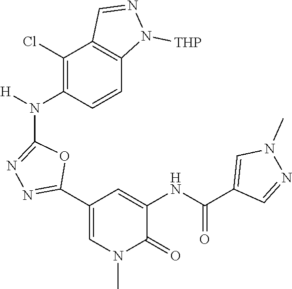

A mixture of 3-bromo-5-[5-[(4-chloro-1-tetrahydropyran-2-yl-indazol-5-yl)amino]-1,3,4-oxadiazol-2-yl]-1-methyl-pyridin-2-one (69.5 mg, 137.42 umol, 1 eq), 1-methylpyrazole-4-carboxamide (22.35 mg, 178.65 umol), Pd(dba)₂ (7.90 mg, 13.74 umol), Cs₂CO₃ (89.55 mg, 274.84 umol) and Xantphos (15.90 mg, 27.48 umol) in dioxane (0.5 mL) was degassed and purged with N₂ for 3 times, and then the mixture was stirred at 100° C. for 16 hr under N₂ atmosphere. The reaction mixture was concentrated under reduced pressure. The residue was purified by preparative eluting with 30% EtOAc in Pet. Ether to give N-[5-[5-[(4-chloro-1-tetrahydropyran-2-yl-indazol-5-yl)amino]-1,3,4-oxadiazol-2-yl]-1-methyl-2-oxo-3-pyridyl]-1-methyl-pyrazole-4-carboxamide (75 mg, 136.37 umol, 99.24% yield) as a white solid. LC-MS (ES⁺, Method A), 0.50 min, m/z 550.2 [M+H]⁺.

### Example 67: N-[5-[5-[(4-chloro-1H-indazol-5-yl)amino]-1,3,4-oxadiazol-2-yl]-1-methyl-2-oxo-3-pyridyl]-1-methyl-pyrazole-4-carboxamide

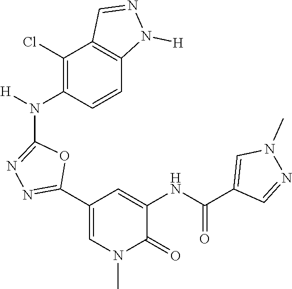 [SMILES: Cn1cc(C(=O)Nc2cc(-c3nnc(Nc4ccc5[nH]ncc5c4Cl)o3)cn(C)c2=O)cn1]

A mixture of N-[5-[5-[(4-chloro-1-tetrahydropyran-2-yl-indazol-5-yl)amino]-1,3,4-oxadiazol-2-yl]-1-methyl-2-oxo-3-pyridyl]-1-methyl-pyrazole-4-carboxamide (75 mg, 136.37 umol) in HCl/dioxane (4 M, 1 mL) was stirred at 25° C. for 2 hr. The reaction mixture was concentrated under reduced pressure. The crude product was triturated with DMF (2 mL) at 25° C. for 30 min and filtered to give N-[5-[5-[(4-chloro-1H-indazol-5-yl)amino]-1,3,4-oxadiazol-2-yl]-1-methyl-2-oxo-3-pyridyl]-1-methyl-pyrazole-4-carboxamide (3.8 mg, 7.57 umol, 5.55% yield, HCl) as a brown solid. LC-MS (ES⁺, Method A), 0.42 min, m/z 466.0 [M+H]⁺. ¹H NMR (400 MHz, DMSO-d₆) δ 13.45 (s, 1H), 9.98 (s, 1H), 9.05 (s, 1H), 8.70 (d, J=2.0 Hz, 1H), 8.46 (s, 1H), 8.13 (s, 2H), 8.07 (d, J=2.0 Hz, 1H), 8.00 (s, 1H), 7.76 (d, J=8.8 Hz, 1H), 7.59 (d, J=8.8 Hz, 1H), 3.89 (s, 3H), 3.64 (s, 3H).

### General Route or the Synthesis of Examples 68 and 69

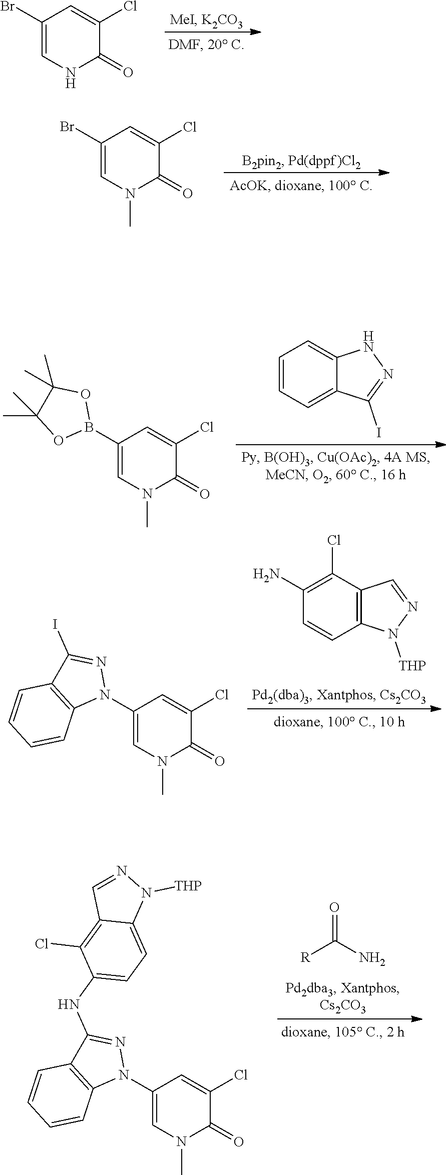

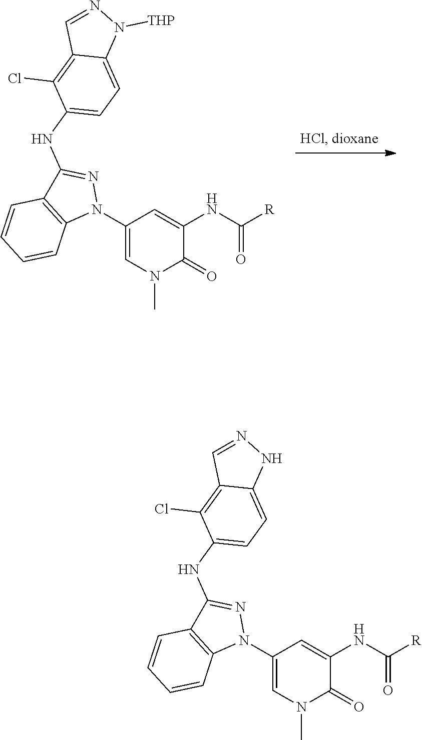

### Step 1: 5-bromo-3-chloro-1-methylpyridin-2(1H)-one

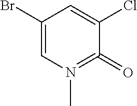

To a solution of 5-bromo-3-chloro-pyridin-2-ol (5 g, 23.99 mmol) in r DMF (50 mL) was added Mel (17.02 g, 119.94 mmol, 7.47 mL) and K₂CO₃ (6.63 g, 47.98 mmol). The mixture was stirred at 20° C. for 1 hr. The reaction mixture was poured into water (100 mL), extracted with ethyl acetate (3×100 mL). The combined organic layers were washed with brine (2×100 mL), dried over Na₂SO₄, filtered and concentrated under reduced pressure to give 5-bromo-3-chloro-1-methylpyridin-2(1H)-one (3.23 g, 14.52 mmol, 60.53% yield) as a white solid. LC-MS (ES⁺, Method A), 1 min, m/z 223.9.

### Step 2: 3-chloro-1-methyl-5-(4,4,5,5-tetramethyl-1,3,2-dioxaborolan-2-yl)pyridin-2(1H)-one

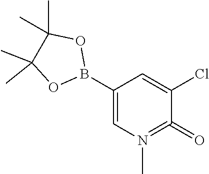

To a solution of 5-bromo-3-chloro-1-methyl-pyridin-2-one (2 g, 8.99 mmol) in dioxane (20 mL) was added 4,4,5,5-tetramethyl-2-(4,4,5,5-tetramethyl-1,3,2-dioxaborolan-2-yl)-1,3,2-dioxaborolane (2.74 g, 10.79 mmol) and Pd(dppf)Cl₂ (657.81 mg, 899.01 umol) at 20° C. under N₂, the mixture was stirred 0.5 hr. And then was added AcOK (2.65 g, 26.97 mmol), the mixture was stirred at 80° C. for 1 hr. The reaction mixture was poured into water (100 mL), extracted with Ethyl acetate (3×25 mL). The combined organic layers were washed with brine (2×20 mL), dried over Na₂SO₄, filtered and concentrated. The residue was purified by flash silica gel chromatography eluting with 0-15% EtOAc in Pet. Ether to give 3-chloro-1-methyl-5-(4,4,5,5-tetramethyl-1,3,2-dioxaborolan-2-yl)pyridin-2(1H)-one (1.3 g, 4.82 mmol, 53.65% yield) as a yellow oil.

### Step 3: 3-chloro-5-(3-iodo-1H-indazol-1-yl)-1-methylpyridin-2(1H)-one

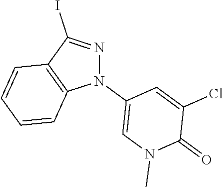 [SMILES: Cn1cc(-n2nc(I)c3ccccc32)cc(Cl)c1=O]

To a solution of 3-chloro-1-methyl-5-(4,4,5,5-tetramethyl-1,3,2-dioxaborolan-2-yl)pyridin-2-one (1.3 g, 4.82 mmol, 1 eq) and 3-iodo-1H-indazole (1.18 g, 4.82 mmol, 1 eq) in MeCN (15 mL) was added Py (763.03 mg, 9.65 mmol, 778.60 uL), boric acid (596.46 mg, 9.65 mmol), Cu(OAc)₂ (1.31 g, 7.23 mmol) and 2 g 4A molecular sieve, then mixture was bubbled with air and stirred at 60° C. for 16 hr. The reaction mixture was poured into water (30 mL), extracted with Ethyl acetate (2×50 mL). The combined organic layers were washed with brine (2×50 mL), dried over Na₂SO₄, filtered and concentrated. The residue was purified by flash silica gel chromatography eluting with 0-10% EtOAc in Pet. Ether to give 3-chloro-1-methyl-5-(4,4,5,5-tetramethyl-1,3,2-dioxaborolan-2-yl)pyridin-2(1H)-one (250 mg, 648.36 umol, 13.44% yield) as a yellow solid.

### Step 4: 3-chloro-5-(3-((4-chloro-1-(tetrahydro-2H-pyran-2-yl)-1H-indazol-5-yl)amino)-1H-indazol-1-yl)-1-methylpyridin-2(1H)-one

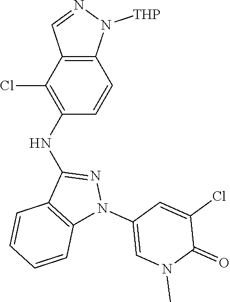

To a mixture of 3-chloro-5-(3-iodoindazol-1-yl)-1-methyl-pyridin-2-one (250 mg, 648.36 umol) and 4-chloro-1-tetrahydropyran-2-yl-indazol-5-amine (163.20 mg, 648.36 umol) in dioxane (3 mL) was added Cs₂CO₃ (422.50 mg, 1.30 mmol), Pd₂(dba)₃ (59.37 mg, 64.84 umol) and Xantphos (75.03 mg, 129.67 umol) in one portion at 25° C. under N₂. The mixture was stirred at 100° C. for 16 hr. The reaction mixture was filtered and the mother solution was concentrated, the residue was added into water (20 mL), extracted with Ethyl acetate (2×15 mL). The combined organic layers were washed with brine (2×10 mL), dried over Na₂SO₄, filtered and concentrated under reduced pressure to give a residue. The residue was purified by flash silica gel chromatography eluting with 0-10% EtOAc in Pet. Ether to give 3-chloro-5-(3-((4-chloro-1-(tetrahydro-2H-pyran-2-yl)-1H-indazol-5-yl)amino)-1H-indazol-1-yl)-1-methylpyridin-2(1H)-one (260 mg, 510.42 umol, 78.72% yield) as a yellow solid.

### Step 5: N-(5-(3-((4-chloro-1-(tetrahydro-2H-pyran-2-yl)-1H-indazol-5-yl)amino)-1H-indazol-1-yl)-1-methyl-2-oxo-1,2-dihydropyridin-3-yl)-2-fluorobenzamide

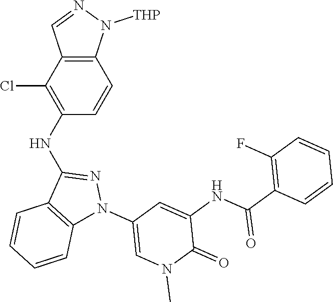

To a solution of 3-chloro-5-[3-[(4-chloro-1-tetrahydropyran-2-yl-indazol-5-yl)amino]indazol-1-yl]-1-methyl-pyridin-2-one (250 mg, 490.79 umol) and 2-fluorobenzamide (75.11 mg, 539.87 umol) in dioxane (2 mL) was added Cs₂CO₃ (319.82 mg, 981.57 umol), Pd₂(dba)₃ (44.94 mg, 49.08 umol), and Xantphos (56.80 mg, 98.16 umol) under N₂, the mixture was stirred at 105° C. for 2 hr. The reaction mixture was filtered and the mother solution was concentrated, the residue was added into water (40 mL), extracted with Ethyl acetate (2×15 mL). The combined organic layers were washed with brine (2×10 mL), dried over Na₂SO₄, filtered and concentrated under reduced pressure to give a residue. The residue was purified by flash silica gel chromatography eluting with 0-20% EtOAc in Pet. Ether to give N-(5-(3-((4-chloro-1-(tetrahydro-2H-pyran-2-yl)-1H-indazol-5-yl)amino)-1H-indazol-1-yl)-1-methyl-2-oxo-1,2-dihydropyridin-3-yl)-2-fluorobenzamide (40 mg, 65.35 umol, 13.32% yield) as a yellow solid.

### Example 68: N-(5-(3-((4-chloro-1H-indazol-5-yl)amino)-1H-indazol-1-yl)-1-methyl-2-oxo-1,2-dihydropyridin-3-yl)-2-fluorobenzamide

To a solution of N-[5-[3-[(4-chloro-1-tetrahydropyran-2-yl-indazol-5-yl)amino]indazol-1-yl]-1-methyl-2-oxo-3-pyridyl]-2-fluoro-benzamide (40 mg, 65.35 umol) in DCM (0.2 mL) was added TFA (616.00 mg, 5.40 mmol, 0.4 mL), the mixture was stirred at 25° C. for 1 hr. The mixture was concentrated under reduced pressure to give a residue. The mixture was triturated with MeOH (5 mL) at 50° C. for 1 h

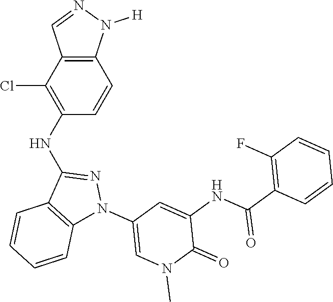 [SMILES: Cn1cc(-n2nc(Nc3ccc4[nH]ncc4c3Cl)c3ccccc32)cc(NC(=O)c2ccccc2F)c1=O]

to give N-(5-(3-((4-chloro-1H-indazol-5-yl)amino)-1H-indazol-1-yl)-1-methyl-2-oxo-1,2-dihydropyridin-3-yl)-2-fluorobenzamide (14.7 mg, 42.06 umol, 16.42% yield) as a yellow solid. LC-MS (ES⁺, Method A), 1 min, m/z 528.3. [M+H]⁺0.1H NMR (400 MHz, DMSO+CF3COOH) δ9.80 (d, J=11.2 Hz, 1H), 8.74 (d, J=2.8 Hz, 1H), 8.06 (s, 1H), 8.01-7.90 (m, 3H), 7.84 (d, J=8.8 Hz, 1H), 7.63 (d, J=8.4 Hz, 2H), 7.55-7.44 (m, 2H), 7.43-7.35 (m, 2H), 7.16 (t, J=7.2 Hz, 1H), 3.65 (s, 3H).
Compounds prepared in a similar manner to that set out above are given below in Table 18

TABLE 18

| Example | Structure | LCMS | ¹H NMR |
|---|---|---|---|
| 69 | 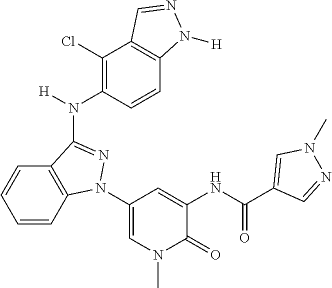 [SMILES: Cn1cc(C(=O)Nc2cc(-n3nc(Nc4ccc5[nH]ncc5c4Cl)c4ccccc43)cn(C)c2=O)cn1] | LC-MS (ES⁺, Method A), 0.45 min, m/z 514.2 [M + H]⁺. | ¹H NMR (400 MHz, DMSO- d₆) δ 13.29 (s, 1H), 9.07 (s, 1H), 8.55 (d, J = 2.8 Hz, 1H), 8.45 (s, 1H), 8.38 (s, 1H), 8.06 (s, 1H), 7.99 (s, 1H), 7.95 (d, J = 8.4 Hz, 1H), 7.86 (d, J = 2.8 Hz, 1H), 7.81 (d, J = 9.2 Hz, 1H), 7.62 (d, J = 8.4 Hz, 1H), 7.56-7.43 (m, 2H), 7.16 (t, J = 7.6 Hz, 1H), 3.89 (s, 3H), 3.63 (s, 3H). |

General method for the synthesis of Example 70:

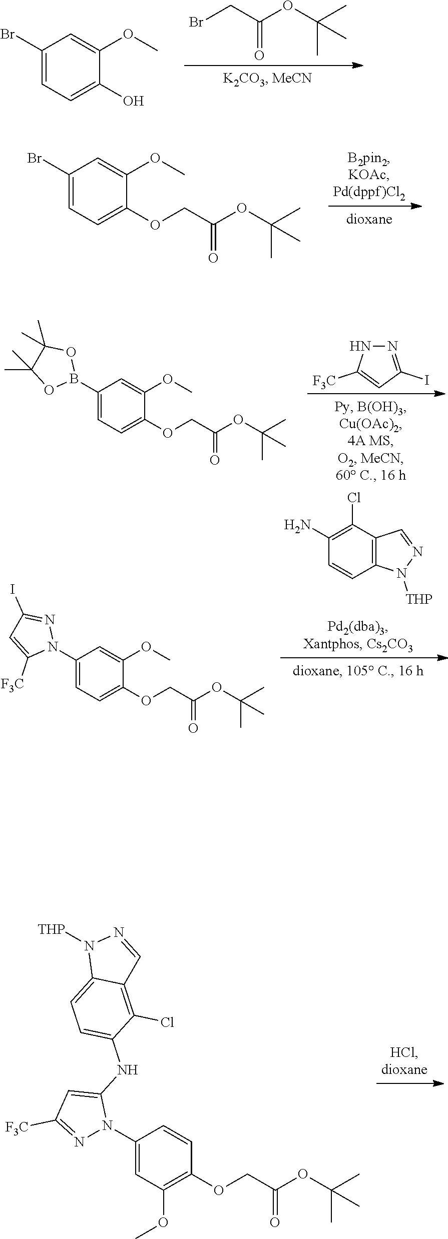

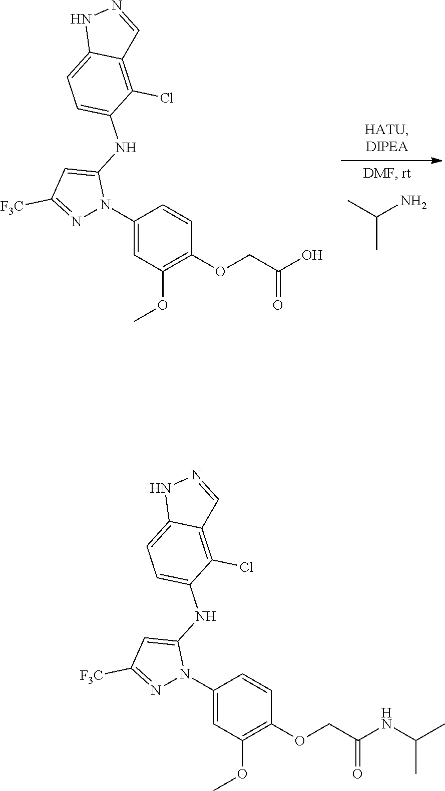

### Step 1: tert-butyl 2-(4-bromo-2-methoxyphenoxy)acetate

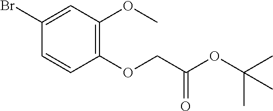 [SMILES: COc1cc(Br)ccc1OCC(=O)OC(C)(C)C]

To a solution of 4-bromo-2-methoxy-phenol (10 g, 49.25 mmol) in MeCN (100 mL) was added K₂CO₃ (13.61 g, 98.51 mmol) and tert-butyl 2-bromoacetate (9.61 g, 49.25 mmol). The mixture was stirred at 80° C. for 16 hr. The reaction mixture was quenched by added water (300 mL), and then diluted with EtOAc (100 mL) and extracted with EtOAc (100 mL×3). The combined organic layers were washed with brine (100 mL), dried over Na₂SO₄, filtered and concentrated under reduced pressure to give a residue. The residue was purified by flash silica gel chromatography eluting with 0-15% EtOAc in Pet. to give tert-butyl 2-(4-bromo-2-methoxyphenoxy)acetate (15.79 g, 48.29 mmol, 98.04% yield) as a white solid.

### Step 2: tert-butyl 2-(2-methoxy-4-(4,4,5,5-tetramethyl-1,3,2-dioxaborolan-2-yl)phenoxy)acetate

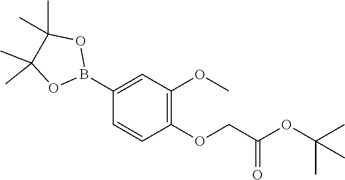 [SMILES: COc1cc(B2OC(C)(C)C(C)(C)O2)ccc1OCC(=O)OC(C)(C)C]

To a solution of tert-butyl 2-(4-bromo-2-methoxyphenoxy)acetate (156 mL) was added 4,4,5,5-tetramethyl-2-(4,4,5,5-tetramethyl-1,3,2-dioxaborolan-2-yl)-1,3,2-dioxaborolane (9.37 g, 36.89 mmol), AcOK (4.83 g, 49.18 mmol) and Pd(dppf)Cl₂ (1.80 g, 2.46 mmol). The mixture was stirred at 90° C. for 16 hr. The reaction mixture was filtered by a bed of celite and the filtrate was concentrated to give the residue. The residue was purified by flash silica gel chromatography eluting with 0-15% EtOAc in Pet. to give tert-butyl 2-(2-methoxy-4-(4,4,5,5-tetramethyl-1,3,2-dioxaborolan-2-yl)phenoxy)acetate (18 g, 45.46 mmol) as a yellow oil.

### Step 3: tert-butyl 2-(2-methoxy-4-(4,4,5,5-tetramethyl-1,3,2-dioxaborolan-2-yl)phenoxy)acetate

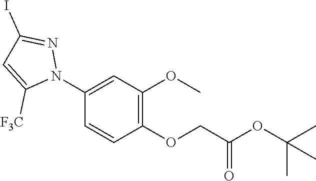 [SMILES: COc1cc(-n2nc(I)cc2C(F)(F)F)ccc1OCC(=O)OC(C)(C)C]

To a solution of tert-butyl 2-(2-methoxy-4-(4,4,5,5-tetramethyl-1,3,2-dioxaborolan-2-yl)phenoxy)acetate (500 mg, 1.37 mmol), 3-iodo-5-(trifluoromethyl)-1H-pyrazole (299.68 mg, 1.14 mmol) in MeCN (7 mL) was added pyridine (180.97 mg, 2.29 mmol), Cu(OAc)₂ (311.67 mg, 1.72 mmol) and boric acid (141.47 mg, 2.29 mmol). The mixture was stirred at 60° C. for 16 hr. The reaction mixture was filtered and the filtrate was concentrated. The residue was purified by flash silica gel chromatography eluting with 0-25% EtOAc in Pet. to give tert-butyl 2-(4-(3-iodo-5-(trifluoromethyl)-1H-pyrazol-1-yl)-2-methoxyphenoxy)acetate (500 mg, 1.00 mmol, 87.73% yield) as a yellow oil.

### Step 4: tert-butyl 2-(4-(5-((4-chloro-1-(tetrahydro-2H-pyran-2-yl)-1H-indazol-5-yl)amino)-3-(trifluoromethyl)-1H-pyrazol-1-yl)-2-methoxyphenoxy)acetate

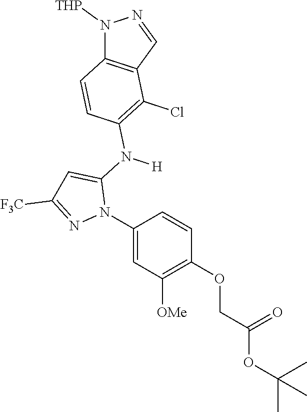

To a mixture of tert-butyl 2-(4-(3-iodo-5-(trifluoromethyl)-1H-pyrazol-1-yl)-2-methoxyphenoxy)acetate (450 mg, 903.19 umol), 4-chloro-1-(tetrahydro-2H-pyran-2-yl)-1H-indazol-5-amine (227.34 mg, 903.19 umol), Pd₂(dba)₃ (82.71 mg, 90.32 umol), Xantphos (104.52 mg, 180.64 umol) and Cs₂CO₃ (588.55 mg, 1.81 mmol) in dioxane (7 mL) was degassed and purged with nitrogen for 3 times, and then the mixture was stirred at 105° C. for 24 hr under nitrogen atmosphere. The reaction mixture was poured into water (100 mL), extracted with Ethyl acetate (60 mL×2). The combined organic layers were washed with brine (60 mL×2), dried over Na₂SO₄, filtered and concentrated. The residue was purified by flash silica gel chromatography eluting with 0-25% EtOAc in Pet. to give tert-butyl 2-(4-(5-((4-chloro-1-(tetrahydro-2H-pyran-2-yl)-1H-indazol-5-yl)amino)-3-(trifluoromethyl)-1H-pyrazol-1-yl)-2-methoxyphenoxy)acetate (240 mg, 385.83 umol, 42.72% yield) as a yellow oil. LC-MS (ES⁺, Method A), 0.666 min, m/z 622.5 [M+H]⁺.

### Step 5: 2-(4-(5-((4-chloro-1H-indazol-5-yl)amino)-3-(trifluoromethyl)-1H-pyrazol-1-yl)-2-methoxyphenoxy)acetic acid

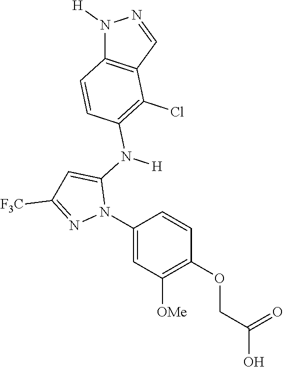 [SMILES: COc1cc(-n2nc(C(F)(F)F)cc2Nc2ccc3[nH]ncc3c2Cl)ccc1OCC(=O)O]

To a solution of tert-butyl 2-(4-(5-((4-chloro-1-(tetrahydro-2H-pyran-2-yl)-1H-indazol-5-yl)amino)-3-(trifluoromethyl)-1H-pyrazol-1-yl)-2-methoxyphenoxy)acetate (50 mg, 80.38 umol) in dioxane (0.5 mL) was added HCl/dioxane (4 M, 0.5 mL). The mixture was stirred 25° C. for 2 hr. The reaction mixture was concentrated under reduced pressure to give 2-(4-(5-((4-chloro-1H-indazol-5-yl)amino)-3-(trifluoromethyl)-1H-pyrazol-1-yl)-2-methoxyphenoxy)acetic acid (50 mg, crude) as a yellow oil.

### Example 70: 2-(4-(5-((4-chloro-1H-indazol-5-yl)amino)-3-(trifluoromethyl)-1H-pyrazol-1-yl)-2-methoxyphenoxy)—N-isopropylacetamide

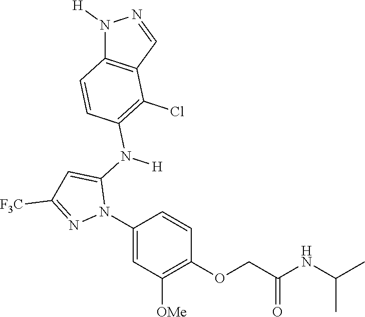 [SMILES: COc1cc(-n2nc(C(F)(F)F)cc2Nc2ccc3[nH]ncc3c2Cl)ccc1OCC(=O)NC(C)C]

To a solution of 2-(4-(5-((4-chloro-1H-indazol-5-yl)amino)-3-(trifluoromethyl)-1H-pyrazol-1-yl)-2-methoxyphenoxy)acetic acid (37.00 mg, 76.79 umol), propan-2-amine (4.99 mg, 84.47 umol), HATU (58.40 mg, 153.59 umol) and DIEA (29.78 mg, 230.38 umol) in DMF (0.5 mL). The mixture was stirred at 20° C. for 2 hr. The reaction mixture was purified by preparative HPLC (42-72% MeCN in H₂O) to give 2-(4-(5-((4-chloro-1H-indazol-5-yl)amino)-3-(trifluoromethyl)-1H-pyrazol-1-yl)-2-methoxyphenoxy)—N-isopropylacetamide (5 mg, 9.06 umol, 11.80% yield) as an off-white solid. LC-MS (ES⁺, Method A), 0.533 min, m/z 523.2 [M+H]⁺. ¹H NMR (400 MHz, DMSO-d₆) δ 13.25 (s, 1H), 7.99 (s, 1H), 7.89 (s, 1H), 7.75 (d, J=7.6 Hz, 1H), 7.40 (d, J=8.8 Hz, 1H), 7.27 (d, J=2.4 Hz, 1H), 7.15 (dd, J=2.4, 8.8 Hz, 1H), 6.99 (dd, J=4.5, 8.8 Hz, 2H), 6.27 (s, 1H), 4.44 (s, 2H), 3.93-378 (m, 1H), 3.78 (s, 3H), 1.07 (s, 3H), 1.05 (s, 3H).

General method for the synthesis of Example 71

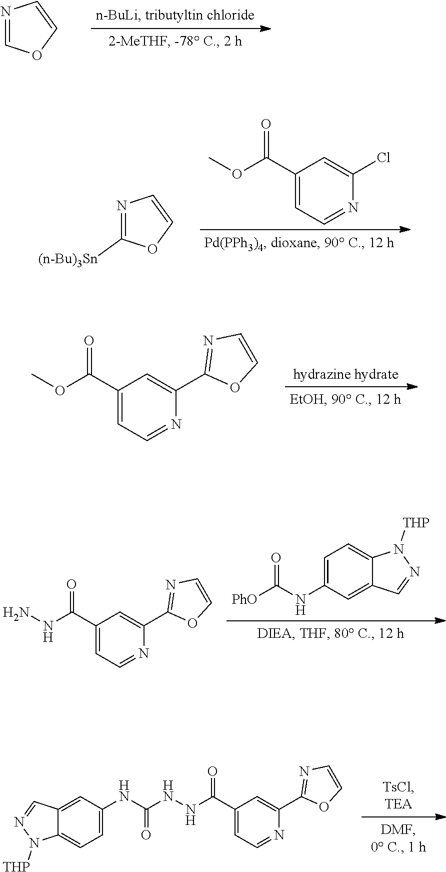

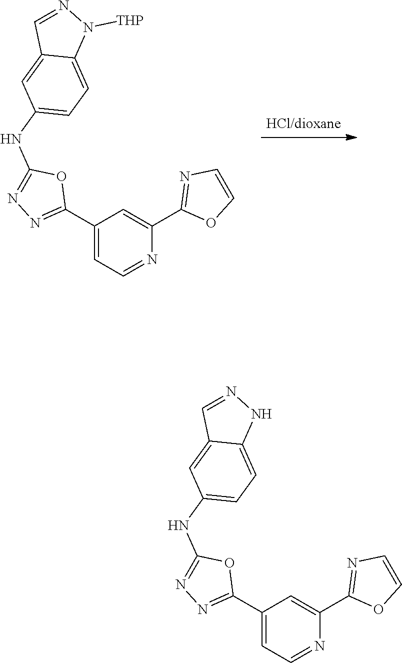

### Step 1: 2-(tributylstannyl)oxazole

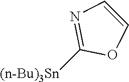

To a solution of oxazole (5 g, 72.40 mmol, 4.63 mL) in 2-MeTHF (200 (n-Bu)_{3S}n mL) at −78° C. under nitrogen was added n-BuLi (2.5 M, 28.96 mL,) slowly, and the mixture was stirred at −78° C. for 0.5 hr, then tributyl(chloro)stannane (23.57 g, 72.40 mmol, 19.48 mL) was added to the mixture and allowed to warm to 25° C. and stirred for 1 hr. The reaction mixture was quenched by added saturated KF aqueous solution (320 mL) at 0° C., and extracted with ethyl acetate (3×300 mL). The combined organic layers were washed with salt water (2×200 mL), dried over anhydrous Na₂SO₄, filtered and concentrated under reduced pressure to give a residue. The solvent was removed under reduced pressure and residue was taken up in Petroleum ether (100 mL). The resulting precipitate was removed by filtration and the filtrate was concentrated under reduced pressure to give 2-(tributylstannyl)oxazole (20 g, 55.85 mmol, 77.14% yield) as a yellow syrup.

### Step 2: methyl 2-(oxazol-2-yl)isonicotinate

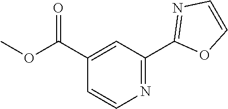

To a mixture of tributyl(oxazol-2-yl)stannane (10 g, 27.92 mmol) ° k and methyl 2-chloropyridine-4-carboxylate (1.60 g, 9.31 mmol) in dioxane (80 mL) was added Pd(PPh₃)₄ (1.08 g, 930.82 umol) at 20° C., then the mixture was stirred at 90° C. for 12 hr. The reaction mixture was pour into water (100 mL), the aqueous phase was extracted with Ethyl acetate (2×80 mL). The combined organic layers were dried over Na₂SO₄, filtered and concentrated under reduced pressure to give a residue. The residue was purified by flash silica gel chromatography eluting with 0-20% EtOAc in Pet. to give methyl 2-(oxazol-2-yl)isonicotinate (400 mg, 1.96 mmol, 21.05% yield) as a white solid.

### Step 3: 2-(oxazol-2-yl)isonicotinohydrazide

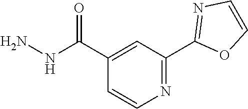 [SMILES: NNC(=O)c1ccnc(-c2ncco2)c1]

To a mixture of methyl 2-(oxazol-2-yl)isonicotinate (400 mg, 1.96 mmol) in MeOH (10 mL) was added N₂H₄·H₂O (1.37 g, 26.77 mmol, 1.33 mL, 98% purity) at 20° C., then the mixture was stirred at 70° C. for 12 hr. The reaction solution was concentrated in vacuum under reduced pressure to give residue to give 2-(oxazol-2-yl) isonicotinohydrazide (180 mg, 881.55 umol, 45.00% yield) as a white solid.

### Step 4: 2-(2-(oxazol-2-yl)isonicotinoyl)-N-(1-(tetrahydro-2H-pyran-2-yl)-1H-indazol-5-yl)hydrazinecarboxamide

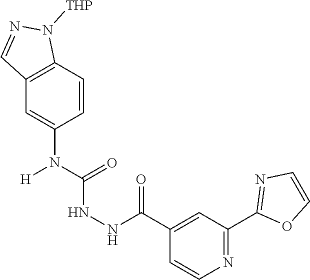

To a solution of 2-(oxazol-2-yl)isonicotinohydrazide (170 mg, 832.58 umol, 1 eq) and phenyl N-(1-tetrahydropyran-2-ylindazol-5-yl)carbamate (337.07 mg, 999.09 umol) in dioxane (5 mL) was DIEA (322.81 mg, 2.50 mmol, 435.06 uL) at 25° C. Then mixture was stirred at 80° C. for 12 hr. The reaction mixture was poured into water (20 mL), extracted with Ethyl acetate (3×20 mL). The combined organic layers were washed with brine (2×20 mL), dried over Na₂SO₄, filtered and concentrated to give 2-(2-(oxazol-2-yl)isonicotinoyl)-N-(1-(tetrahydro-2H-pyran-2-yl)-1H-indazol-5-yl)hydrazinecarboxamide (200 mg, 446.98 umol, 53.69% yield) as a yellow solid.

### Step 5: 5-(2-(oxazol-2-yl)pyridin-4-yl)-N-(1-(tetrahydro-2H-pyran-2-yl)-1H-indazol-5-yl)-1,3,4-oxadiazol-2-amine

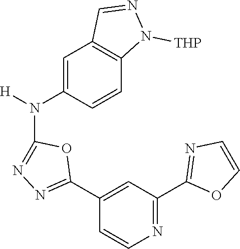

To a solution of 2-(2-(oxazol-2-yl)isonicotinoyl)-N-(1-(tetrahydro-2H-pyran-2-yl)-1H-indazol-5-yl)hydrazinecarboxamide (120 mg, 268.19 umol) in DCM (3 mL) and DMF (1 mL) was added TEA (81.41 mg, 804.57 umol, 111.99 uL) at 20° C. and then the mixture was cooled to⁰° C. and then TosCl (56.24 mg, 295.01 umol) was added in the mixture and the mixture was stirred at 0° C. for 2 hr. The mixture was dissolved in water (20 mL) and extracted with Ethyl acetate (2×50 mL). The combined organic layer was washed with saturated NaCl (3×3=20 mL), dried over Na₂SO₄, filtered and concentrated under reduced pressure to give 5-(2-(oxazol-2-yl)pyridin-4-yl)-N-(1-(tetrahydro-2H-pyran-2-yl)-1H-indazol-5-yl)-1,3,4-oxadiazol-2-amine (110 mg, 256.15 umol, 95.51% yield) as a brown solid.

### Example 71: N-(1H-indazol-5-yl)-5-(2-(oxazol-2-yl)pyridin-4-yl)-1,3,4-oxadiazol-2-amine

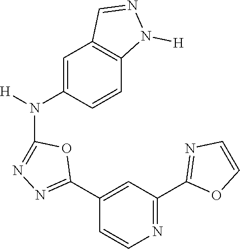 [SMILES: c1cc(-c2nnc(Nc3ccc4[nH]ncc4c3)o2)cc(-c2ncco2)n1]

To a solution of 5-(2-(oxazol-2-yl)pyridin-4-yl)-N-(1-(tetrahydro-2H-pyran-2-yl)-1H-indazol-5-yl)-1,3,4-oxadiazol-2-amine (110 mg, 256.15 umol) in DCM (1 mL) was added TFA (770.00 mg, 6.75 mmol, 0.5 mL) at 20° C. and then the mixture was stirred at 20° C. for 30 min. The mixture was concentrated under reduced pressure to give a residue. The residue was purified by preparative HPLC (12-42% MeCN in H₂O) to give N-(1H-indazol-5-yl)-5-(2-(oxazol-2-yl)pyridin-4-yl)-1,3,4-oxadiazol-2-amine (14.7 mg, 42.06 umol, 16.42% yield) as a yellow solid. LC-MS (ES⁺, Method A), 0.426 min, m/z 345.8 [M+H]⁺. ¹H NMR (400 MHz, DMSO-d₆) δ 13.04 (br s, 1H), 9.09-8.85 (m, 1H), 8.48 (s, 1H), 8.39 (s, 1H), 8.16 (d, J=1.2 Hz, 1H), 8.09 (s, 1H), 7.94 (dd, J=1.6, 5.2 Hz, 1H), 7.61-7.46 (m, 3H).

General method for the synthesis of Example 72

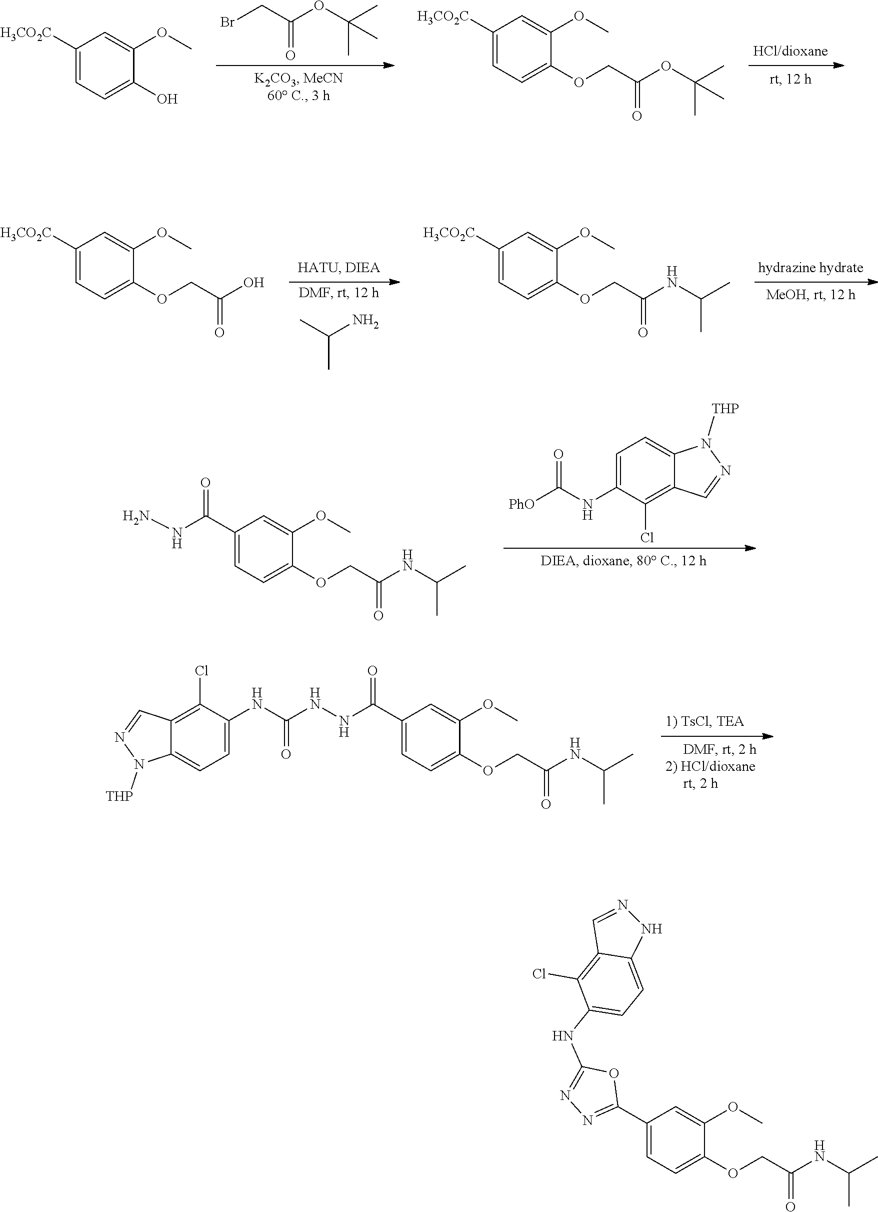

### Step 1: methyl 4-(2-tert-butoxy-2-oxo-ethoxy)-3-methoxy-benzoate

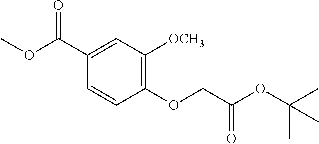 [SMILES: COC(=O)c1ccc(OCC(=O)OC(C)(C)C)c(OC)c1]

To a solution of methyl 4-hydroxy-3-methoxy-benzoate (3 g, 16.47 mmol), tert-butyl 2-bromoacetate (6.42 g, 32.94 mmol, 4.87 mL) in MeCN (15 mL) was added K₂CO₃ (4.55 g, 32.94 mmol). The mixture was stirred at 60° C. for 2 hr. The reaction mixture was diluted with water (50 mL) and extracted with EtOAc (3×50 mL). The combined organic layers dried over Na₂SO₄, filtered and concentrated under reduced pressure to give a residue. The residue was purified by flash silica gel chromatography eluting with 0-25% EtOAc in Pet. Ether to give methyl 4-(2-tert-butoxy-2-oxo-ethoxy)-3-methoxy-benzoate (4.5 g, 15.19 mmol, 92.22% yield) as a white solid. LC-MS (ES⁺, Method A), 0.47 min, m/z 296.3 [M+H]⁺.

### Step 2: 2-(2-methoxy-4-methoxycarbonyl-phenoxy)acetic acid

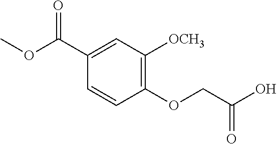 [SMILES: COC(=O)c1ccc(OCC(=O)O)c(OC)c1]

A mixture of methyl 4-(2-tert-butoxy-2-oxo-ethoxy)-3-methoxy-benzoate (4.5 g, 15.19 mmol) in HCl/dioxane (30 mL) was stirred at 25° C. for 2 hr. The reaction mixture concentrated under reduced pressure to give 2-(2-methoxy-4-methoxycarbonyl-phenoxy)acetic acid (4 g, crude) as a white solid, which was used directly in the next step without further purification. LC-MS (ES⁺, Method A), 0.33 min, m/z 241.0 [M+H]⁺.

### Step 3: methyl 4-[2-(isopropylamino)-2-oxo-ethoxy]-3-methoxy-benzoate

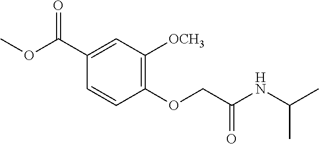 [SMILES: COC(=O)c1ccc(OCC(=O)NC(C)C)c(OC)c1]

To a solution of 2-(2-methoxy-4-methoxycarbonyl-phenoxy)acetic acid (4 g, 16.65 mmol), propan-2-amine (1.97 g, 33.30 mmol, 2.86 mL, 2 eq) in DMF (30 mL) was added HATU (9.50 g, 24.98 mmol) and DIEA (10.76 g, 83.26 mmol, 14.50 mL). The mixture was stirred at 25° C. for 2 hr. The reaction mixture was diluted with water (50 mL) and extracted with EtOAc (3×50 mL). The combined organic layers dried over Na₂SO₄, filtered and concentrated under reduced pressure to give a residue. The residue was purified by flash silica gel chromatography eluting with 0-50% EtOAc in Pet. Ether to give methyl 4-[2-(isopropylamino)-2-oxo-ethoxy]-3-methoxy-benzoate (6 g, crude) as a white solid. LC-MS (ES⁺, Method A), 0.37 min, m/z 282.0 [M+H]⁺.

### Step 4: 2-[4-(hydrazinecarbonyl)-2-methoxy-phenoxy]-N-isopropyl-acetamide

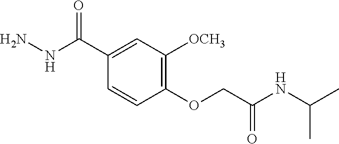 [SMILES: COc1cc(C(=O)NN)ccc1OCC(=O)NC(C)C]

To a solution of methyl 4-[2-(isopropylamino)-2-oxo-ethoxy]-3-methoxy-benzoate (3 g, 10.66 mmol) in MeOH (30 mL) was added hydrazine hydrate (5.1 g, 101.88 mmol, 4.95 mL). The mixture was stirred at 25° C. for 2 hr. The reaction mixture was concentrated under reduced pressure to give 2-[4-(hydrazinecarbonyl)-2-methoxy-phenoxy]-N-isopropyl-acetamide (1.5 g, 5.33 mmol, 50.00% yield) as a white solid. LC-MS (ES⁺, Method A), 0.25 min, m/z 281.9 [M+H]⁺.

### Step 5: 2-[4-[[(4-chloro-1-tetrahydropyran-2-yl-indazol-5-yl)carbamoylamino]carbamoyl]-2-methoxy-phenoxy]-N-isopropyl-acetamide

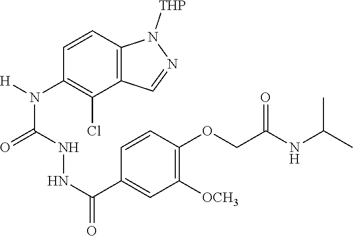

To a solution of 2-[4-(hydrazinecarbonyl)-2-methoxy-phenoxy]-N-isopropyl-acetamide (300 mg, 1.07 mmol), phenyl N-(4-chloro-1 tetrahydropyran-2-yl-indazol-5-yl)carbamate (396.52 mg, 1.07 mmol) in dioxane (5 mL) was added DIEA (413.49 mg, 3.20 mmol, 557.27 uL). The mixture was stirred at 80° C. for 16 hr. The reaction mixture was partitioned between H₂O (10 mL) and EtOAc (10 mL). The organic phase was separated, dried over anhydrous Na₂SO₄ and concentrated under reduced pressure to give 2-[4-[[(4-chloro-1-tetrahydropyran-2-yl-indazol-5-yl)carbamoylamino]carbamoyl]-2-methoxy-phenoxy]-N-isopropyl-acetamide (480 mg, 858.66 umol, 80.52% yield) as white solid. LC-MS (ES⁺, Method A), 0.39 min, m/z 559.1 [M+H]⁺.

### Step 6: 2-[4-[5-[(4-chloro-1-tetrahydropyran-2-yl-indazol-5-yl)amino]-1,3,4-Oxadiazol-2-yl]-2-methoxy-phenoxy]-N-isopropyl-acetamide

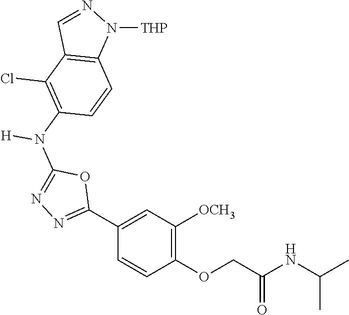

To a solution of 2-[4-[[(4-chloro-1-tetrahydropyran-2-yl-indazol-5-yl)carbamoylamino]carbamoyl]-2-methoxy-phenoxy]-N-isopropyl-acetamide (100 mg, 178.89 umol) in DMF (1 mL) was added TosCl (85.26 mg, 447.22 umol) and TEA (54.30 mg, 536.66 umol, 74.70 uL). The mixture was stirred at 25° C. for 2 hr. The reaction mixture was concentrated under reduced pressure to give a residue. The residue was purified by preparative HPLC (43-73% MeCN in H₂O) to give 2-[4-[5-[(4-chloro-1-tetrahydropyran-2-yl-indazol-5-yl)amino]-1,3,4-oxadiazol-2-yl]-2-methoxy-phenoxy]-N-isopropyl-acetamide (20 mg, 36.97 umol, 20.67% yield) as a white solid. LC-MS (ES⁺, Method A), 0.47 min, m/z 541.1 [M+H]⁺.

### Example 72: 2-[4-[5-[(4-chloro-1H-indazol-5-yl)amino]-1,3,4-oxadiazol-2-yl]-2-methoxy-phenoxy]-N-isopropyl-acetamide

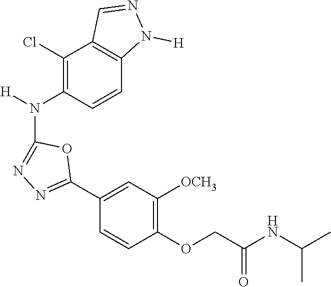 [SMILES: COc1cc(-c2nnc(Nc3ccc4[nH]ncc4c3Cl)o2)ccc1OCC(=O)NC(C)C]

A mixture of 2-[4-[5-[(4-chloro-1-tetrahydropyran-2-yl-indazol-5-yl)amino]-1,3,4-oxadiazol-2-yl]-2-methoxy-phenoxy]-N-isopropyl-acetamide (20 mg, 36.97 umol, 1 eq) in HCl/dioxane (4 M, 2 mL) was stirred at 25° C. for 1 hr. The reaction mixture was concentrated under reduced pressure to give a residue. The residue was purified by preparative HPLC (22-52% MeCN in H₂O) to give 2-[4-[5-[(4-chloro-1H-indazol-5-yl)amino]-1,3,4-oxadiazol-2-yl]-2-methoxy-phenoxy]-N-isopropyl-acetamide (10 mg, 21.37 umol, 57.82% yield, 97.655% purity) as a white solid. LC-MS (ES⁺, Method A), 0.40 min, m/z 457.1 [M+H]⁺. ¹H NMR (400 MHz, DMSO-d₆) δ 13.45 (s, 1 H), 10.06-9.89 (m, 1H), 8.12 (s, 1H), 7.85-7.75 (m, 2H), 7.59 (d, J=8.8 Hz, 1H), 7.42 (d, J=2.0 Hz, 1H), 7.37 (dd, J=2.0, 8.4 Hz, 1H), 7.05 (d, J=8.0 Hz, 1H), 4.53 (s, 2H), 3.96-3.83 (m, 4H), 1.09 (d, J=6.4 Hz, 6H).

General method for the synthesis of Example 73

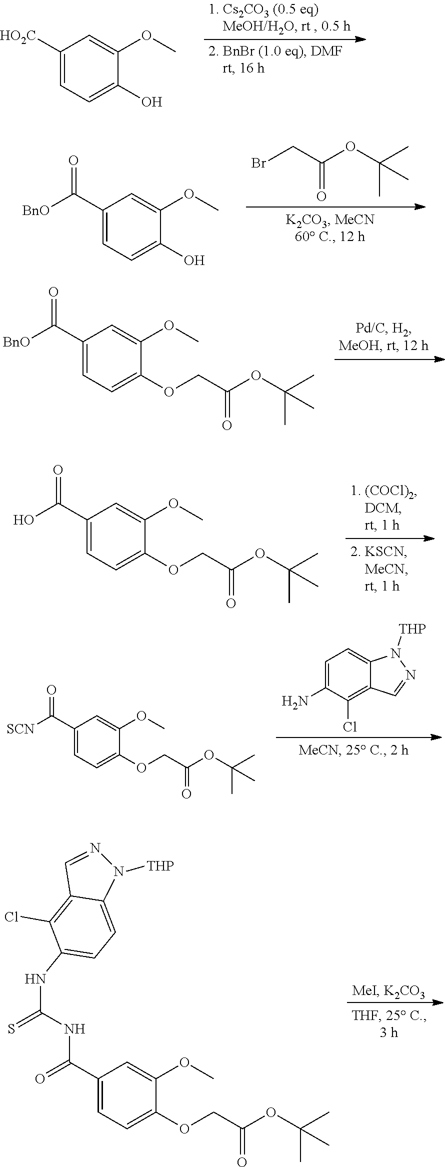

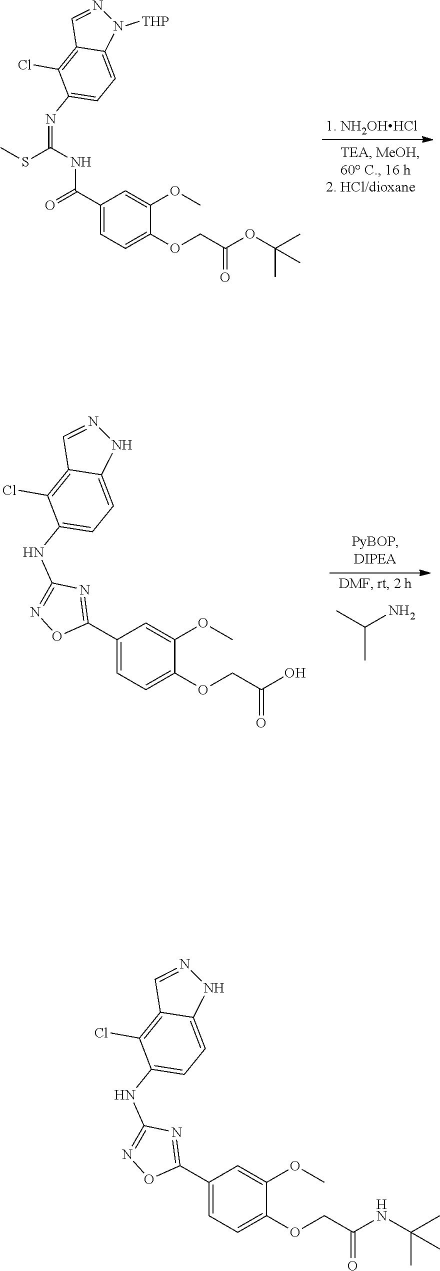

### Step 1: benzyl 4-hydroxy-3-methoxybenzoate

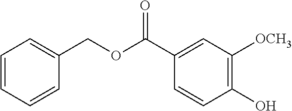 [SMILES: COc1cc(C(=O)OCc2ccccc2)ccc1O]

To a solution of 4-hydroxy-3-methoxy-benzoic acid (2 g, 11.89 mmol, 1 eq) in MeOH (5 mL) was added Cs₂CO₃ (1.94 g, 5.95 mmol). The mixture was stirred at 25° C. for 0.5 hr. Then BnBr (2.03 g, 11.89 mmol, 1.41 mL) was added to the reaction mixture at 0° C., and the mixture was stirred at 25° C. for 10 hr. The reaction mixture was diluted with water (40 mL) and extracted with EtOAc (40 mL*3). The combined organic layers were dried over Na₂SO₄, filtered and concentrated under reduced pressure to give a residue. The residue was purified by flash silica gel chromatography eluting with 0-25% EtOAc in Pet. Ether to give benzyl 4-hydroxy-3-methoxy-benzoate (2 g, 7.74 mmol, 65.11% yield) as a colorless oil. LC-MS (ES⁺, Method A), 0.42 min, m/z 259.1 [M+H]⁺.

### Step 2: benzyl 4-(2-(tert-butoxy)-2-oxoethoxy)-3-methoxybenzoate

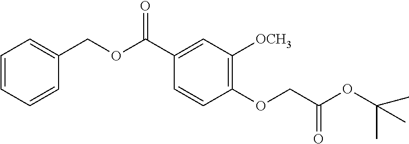 [SMILES: COc1cc(C(=O)OCc2ccccc2)ccc1OCC(=O)OC(C)(C)C]

To a solution of benzyl 4-hydroxy-3-methoxy-benzoate (1 g, 3.87 mmol, 1 eg), tert-butyl 2-bromoacetate (1.51 g, 7.74 mmol, 1.14 mL) in MeCN (10 mL) was added K₂CO₃ (1.07 g, 7.74 mmol). The mixture was stirred at 60° C. for 12 hr. The reaction mixture was concentrated under reduced pressure to give a residue. The residue was purified by flash silica gel chromatography eluting with 0-25% EtOAc in Pet. Ether to give benzyl 4-(2-tert-butoxy-2-oxo-ethoxy)-3-methoxy-benzoate (1.4 g, 3.76 mmol, 97.09% yield) as a colorless oil. LC-MS (ES⁺, Method A), 0.56 min, m/z 372.9 [M+H]⁺.

### Step 3: 4-(2-(tert-butoxy)-2-oxoethoxy)-3-methoxybenzoic acid

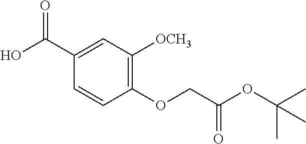 [SMILES: COc1cc(C(=O)O)ccc1OCC(=O)OC(C)(C)C]

A mixture of benzyl 4-(2-tert-butoxy-2-oxo-ethoxy)-3-methoxy-benzoate (1.4 g, 3.76 mmol), Pd/C (500 mg, 10% purity) in MeOH (20 mL) was degassed and purged with Ar for 3 times, and then the mixture was stirred at 25° C. for 16 hr under H₂ (15 psi) atmosphere. The reaction mixture was filtered and concentrated under reduced pressure to give 4-(2-tert-butoxy-2-oxo-ethoxy)-3-methoxy-benzoic acid (1 g, 3.54 mmol, 94.23% yield) as a white solid. LC-MS (ES⁺, Method A), 0.41 min, m/z 282.9 [M+H]⁺.

### Step 4: tert-butyl 2-(4-chlorocarbonyl-2-methoxy-phenoxy)acetate

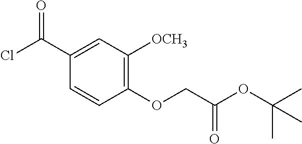 [SMILES: COc1cc(C(=O)Cl)ccc1OCC(=O)OC(C)(C)C]

To a solution of 4-(2-tert-butoxy-2-oxo-ethoxy)-3-methoxy-benzoic acid (1 g, 3.54 mmol) in DCM (5 mL) was added (COCl)₂ (899.26 mg, 7.08 mmol, 620.18 uL). The mixture was stirred at 25° C. for 2 hr. The reaction mixture was concentrated under reduced pressure to give tert-butyl 2-(4-chlorocarbonyl-2-methoxy-phenoxy)acetate (1 g, 3.33 mmol, 93.87% yield) as a yellow solid.

### Step 5: tert-butyl 2-(4-carbonisothiocyanatidoyl-2-methoxy-phenoxy)acetate

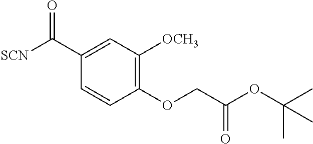

To a solution of tert-butyl 2-(4-chlorocarbonyl-2-methoxy-phenoxy)acetate (1 g, 3.33 mmol) in MeCN (10 mL) was added thiocyanatopotassium (484.72 mg, 4.99 mmol, 484.72 uL). The mixture was stirred at 25° C. for 1 hr. The solvent was evaporated to give tert-butyl 2-(4-carbonisothiocyanatidoyl-2-methoxy-phenoxy)acetate (1 g, 3.09 mmol, 93.00% yield) as a brown solid, which was used directly in the next step without further purification. LC-MS (ES⁺, Method A), 0.48 min, m/z 346.0 [M+Na]⁺.

### Step 6: tert-butyl 2-[4-[(4-chloro-1-tetrahydropyran-2-yl-indazol-5-yl) carbamothioyl-carbamoyl]-2-methoxy-phenoxy]acetate

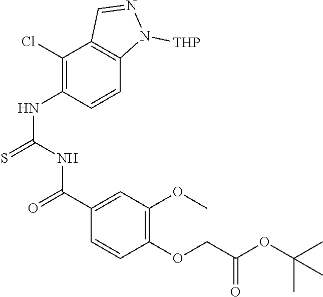

To a solution of tert-butyl 2-(4-carbonisothiocyanatidoyl-2-methoxy-phenoxy)acetate (1 g, 3.09 mmol) in MeCN (10 mL) was added 4-chloro-1-tetrahydropyran-2-yl-indazol-5-amine (700.57 mg, 2.78 mmol). The mixture was stirred at 25° C. for 2 hr. The reaction mixture was filtered and concentrated under reduced pressure to give a residue. The residue was purified by flash silica gel chromatography eluting with 0-25% EtOAc in Pet. Ether to give tert-butyl 2-[4-[(4-chloro-1-tetrahydropyran-2-yl-indazol-5-yl)carbamothioylcarbamoyl]-2-methoxy-phenoxy]acetate (1.2 g, 2.09 mmol, 80.00% yield) as a yellow solid. LC-MS (ES⁺, Method A), 0.58 min, m/z 575.1 [M+H]⁺.

### Step 7: tert-butyl 2-[4-[[(E)—N-(4-chloro-1-tetrahydropyran-2-yl-indazol-5-yl)—C-methylsulfanyl-carbonimidoyl]carbamoyl]-2-methoxy-phenoxy]acetate

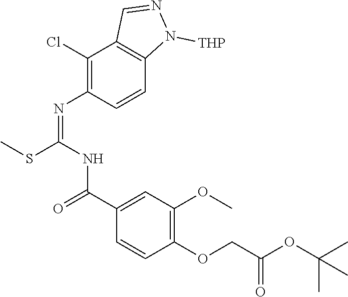

To a solution of tert-butyl 2-[4-[(4-chloro-1-tetrahydropyran-2-yl-indazol-5-yl)carbamothioylcarbamoyl]-2-methoxy-phenoxy]acetate (900 mg, 1.57 mmol, 1 eq), Mel (1.11 g, 7.83 mmol, 487.14 uL, 5 eq) in THE (10 mL) was added K₂CO₃ (432.59 mg, 3.13 mmol, 2 eq). The mixture was stirred at 25° C. for 2 hr. The reaction mixture was concentrated under reduced pressure to give tert-butyl 2-[4-[[(E)—N-(4-chloro-1-tetrahydropyran-2-yl-indazol-5-yl)—C-methylsulfanyl-carbonimidoyl] carbamoyl]-2-methoxy-phenoxy]acetate (900 mg, 1.53 mmol, 97.62% yield) as a yellow solid, which was used directly in the next step without further purification. LC-MS (ES⁺, Method A), 0.60 min, m/z 589.2 [M+H]⁺.

### Step 8: tert-butyl 2-(4-chlorocarbonyl-2-methoxy-phenoxy)acetate

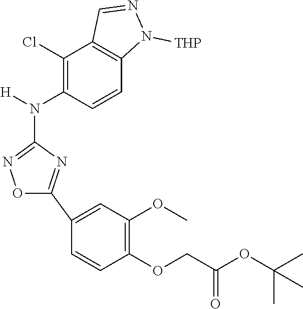

To a solution of tert-butyl 2-[4-[[(E)—N-(4-chloro-1-tetrahydropyran-2-yl-indazol-5-yl)—C-methylsulfanyl-carbonimidoyl]carbamoyl]-2-methoxy-phenoxy]acetate (900 mg, 1.53 mmol) in MeOH (10 mL) was added NH₂OH·HCl (530.82 mg, 7.64 mmol) and TEA (1.08 g, 10.69 mmol, 1.49 mL). The mixture was stirred at 60° C. for 16 hr. The reaction mixture was concentrated under reduced pressure to give a residue. The residue was purified by flash silica gel chromatography eluting with 0-25% EtOAc in Pet. Ether to give tert-butyl 2-[4-[3-[(4-chloro-1-tetrahydropyran-2-yl-indazol-5-yl)amino]-1,2,4-oxadiazol-5-yl]-2-methoxy-phenoxy]acetate (290 mg, 521.57 umol, 34.14% yield) as a yellow solid. LC-MS (ES⁺, Method A), 0.8 min, m/z 589.2 [M+H]⁺.

### Step 9: 2-[4-[3-[(4-chloro-1H-indazol-5-yl)amino]-1,2,4-oxadiazol-5-yl]-2-methoxy-phenoxy]acetic acid

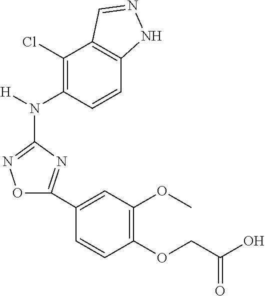 [SMILES: COc1cc(-c2nc(Nc3ccc4[nH]ncc4c3Cl)no2)ccc1OCC(=O)O]

A mixture of tert-butyl 2-[4-[3-[(4-chloro-1-tetrahydropyran-2-yl-indazol-5-yl)amino]-1,2,4-oxadiazol-5-yl]-2-methoxy-phenoxy]acetate (240 mg, 431.65 umol, 1 eq) in HCl/dioxane (5 mL) was stirred at 25° C. for 16 hr. The reaction mixture was concentrated under reduced pressure to give 2-[4-[3-[(4-chloro-1H-indazol-5-yl)amino]-1,2,4-oxadiazol-5-yl]-2-methoxy-phenoxy]acetic acid (180 mg, crude) as a yellow solid, which was used directly in the next step without further purification. LC-MS (ES⁺, Method A), 0.41 min, m/z 415.9 [M+H]⁺.

### Example 73: 2-[4-[3-[(4-chloro-1H-indazol-5-yl)amino]-1,2,4-oxadiazol-5-yl]-2-methoxy-phenoxy]-N-isopropyl-acetamide

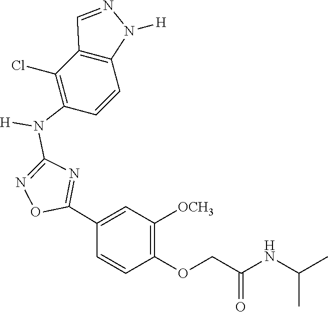 [SMILES: COc1cc(-c2nc(Nc3ccc4[nH]ncc4c3Cl)no2)ccc1OCC(=O)NC(C)C]

To a solution of 2-[4-[3-[(4-chloro-1H-indazol-5-yl)amino]-1,2,4-oxadiazol-5-yl]-2-methoxy-phenoxy]acetic acid (180 mg, 398.01 umol, 1 eq, HCl) propan-2-amine (47.05 mg, 796.02 umol, 68.39 uL) in DMF (1 mL) was added PyBOP (414.24 mg, 796.02 umol) and DIEA (257.20 mg, 1.99 mmol, 346.62 uL). The mixture was stirred at 25° C. for 2 hr. The reaction mixture was concentrated under reduced pressure to give a residue. The residue was purified by preparative HPLC (19-49% MeCN in H₂O) to give 2-[4-[3-[(4-chloro-1H-indazol-5-yl)amino]-1,2,4-oxadiazol-5-yl]-2-methoxy-phenoxy]-N-isopropyl-acetamide (10.9 mg, 21.07 umol, 5.29% yield, HCl salt) as a white solid. LC-MS (ES⁺, Method A), 0.47 min, m/z 456.9 [M+H]⁺. ¹H NMR (400 MHz, DMSO-d₆) δ 13.40 (s, 1H), 9.11 (s, 1H), 8.10 (s, 1H), 7.86 (br d, J=7.6 Hz, 1H), 7.69-7.59 (m, 2H), 7.58-7.52 (m, 2H), 7.07 (d, J=8.8 Hz, 1H), 4.57 (s, 2H), 3.89 (m, 4H), 1.09 (d, J=6.8 Hz, 6H).

General method for the synthesis of Example 74

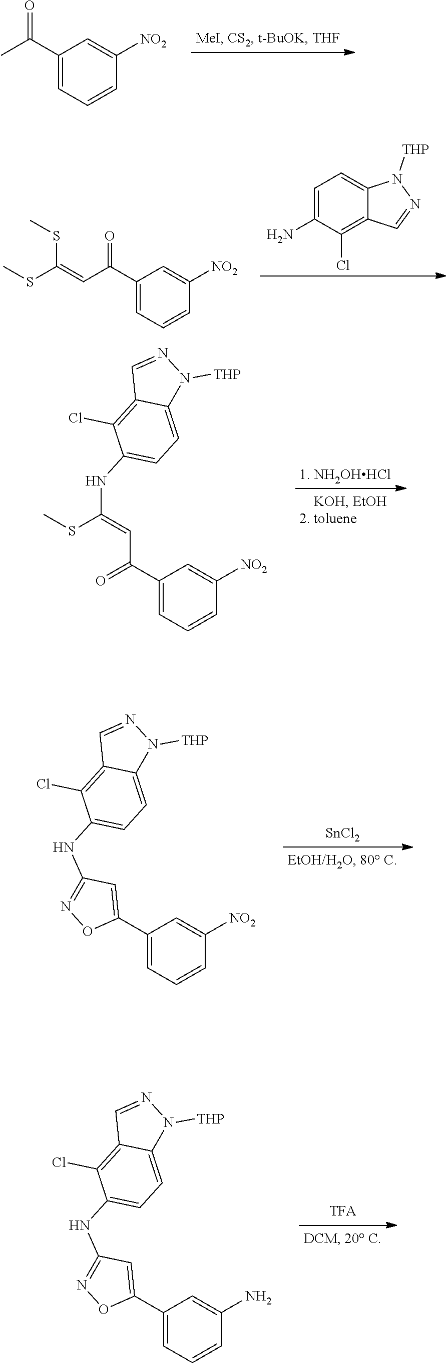

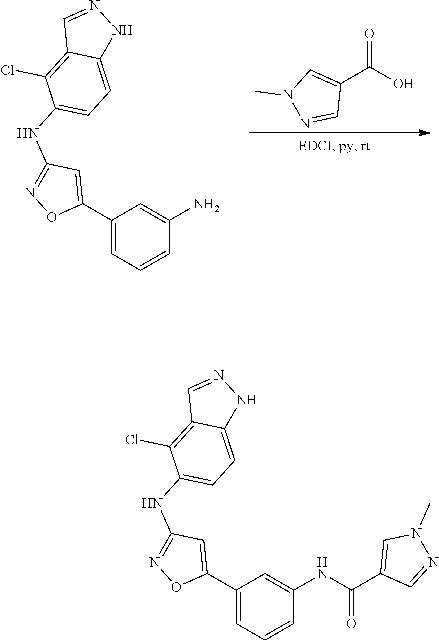

### Step 1: 3,3-bis(methylthio)-1-(3-nitrophenyl)prop-2-en-1-one

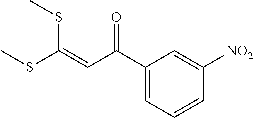 [SMILES: CSC(=CC(=O)c1cccc([N+](=O)[O-])c1)SC]

To the solution of 1-(3-nitrophenyl)ethanone (5 g, 30.28 mmol) and CS₂ (5.07 g, 66.61 mmol, 4.03 mL) in THE (60 mL) was added t-BuOK (1 M, 66.61 mL) at 0° C. under nitrogen. The mixture was stirred at 20° C. for 0.5 hr. Mel (21.49 g, 151.38 mmol, 9.42 mL) was added to the mixture and the mixture was stirred at 20° C. for 0.5 hr. The reaction mixture was diluted with H₂O (5 mL) and extracted with ethyl acetate (50 mL×3). The combined organic layers were washed with brine (100 mL), dried over Na₂SO₄, filtered and concentrated under reduced pressure to give a residue. The residue was purified by column chromatography eluting with 0-50% EtOAc in Pet. Ether to give 3,3-bis(methylthio)-1-(3-nitrophenyl)prop-2-en-1-one (2 g, 7.43 mmol, 24.53% yield) as a yellow solid.

### Step 2: (Z)-3-((4-chloro-1-(tetrahydro-2H-pyran-2-yl)-1H-indazol-5-yl)amino)-3-(methylthio)-1-(3-nitrophenyl)prop-2-en-1-one

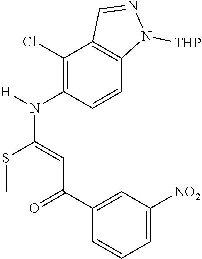

To a mixture of 3,3-bis(methylthio)-1-(3-nitrophenyl)prop-2-en-1-one (500 mg, 1.86 mmol), 4-chloro-1-(tetrahydro-2H-pyran-2-yl)-1H-indazol-5-amine (467.28 mg, 1.86 mmol) in toluene (5 mL) was stirred at 120° C. for 36 hr. The mixture was concentrated under reduce pressure to give a residue then was triturate with DMF (3 mL) at 40° C. for 1 h and filtered to give (Z)-3-((4-chloro-1-(tetrahydro-2H-pyran-2-yl)-1H-indazol-5-yl)amino)-3-(methylthio)-1-(3-nitrophenyl)prop-2-en-1-one (328 mg, 693.53 umol, 41.00% yield) as a brown solid.

### Step 3: N-(4-chloro-1-(tetrahydro-2H-pyran-2-yl)-1H-indazol-5-yl)-5-(3-nitrophenyl)isoxazol-3-amine

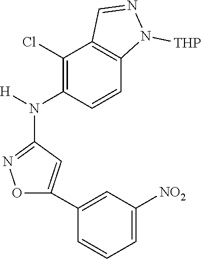

To a solution of (Z)-3-[(4-chloro-1-tetrahydropyran-2-yl-indazol-5-yl)amino]-3-methylsulfanyl-1-(3-nitrophenyl)prop-2-en-1-one (320 mg, 676.61 umol) in EtOH (4 mL) was added NH₂OH·HCl (188.07 mg, 2.71 mmol), KOH (151.85 mg, 2.71 mmol) at 20° C. and the mixture was stirred at 80° C. for 2 hr. The mixture was concentrated under reduced pressure to give a residue. Then to a solution of the residue in toluene (4 mL) was stirred at 110° C. for 3 h. The mixture was concentrated under reduced pressure to give a residue. The residue was purified by preparative HPLC (54-84% MeCN in H₂O) to give N-(4-chloro-1-(tetrahydro-2H-pyran-2-yl)-1H-indazol-5-yl)-5-(3-nitrophenyl)isoxazol-3-amine (60 mg, 136.41 umol, 20.48% yield) as a brown solid.

### Step 4: 5-(3-aminophenyl)-N-(4-chloro-1-(tetrahydro-2H-pyran-2-yl)-1H-indazol-5-yl)isoxazol-3-amine

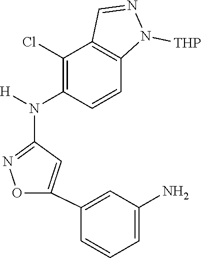

To a solution of N-(4-chloro-1-(tetrahydro-2H-pyran-2-yl)-1H-indazol-5-yl)-5-(3-nitrophenyl)isoxazol-3-amine (60 mg, 136.41 umol) in EtOH (2 mL) and H₂O (0.2 mL) was added SnCl₂.2H₂O (61.56 mg, 272.82 umol) at 20° C. and then the mixture was stirred at 80° C. for 2 hr. The mixture was dissolved in water (20 mL) and extracted with ethyl acetate (30 mL×2). The combined organic layer was dried over Na₂SO₄, filtered and concentrated under reduced pressure to give 5-(3-aminophenyl)-N-(4-chloro-1-(tetrahydro-2H-pyran-2-yl)-1H-indazol-5-yl)isoxazol-3-amine (55 mg, 134.19 umol, 98.37% yield) as a brown oil.

### Step 5: 5-(3-aminophenyl)-N-(4-chloro-1H-indazol-5-yl)isoxazol-3-amine

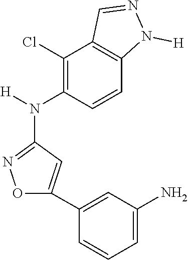 [SMILES: Nc1cccc(-c2cc(Nc3ccc4[nH]ncc4c3Cl)no2)c1]

To a solution of 5-(3-aminophenyl)-N-(4-chloro-1-(tetrahydro-2H-pyran-2-yl)-1H-indazol-5-yl)isoxazol-3-amine (55 mg, 134.19 umol) in DCM (0.5 mL) was added TFA (2.82 g, 24.76 mmol, 1.83 mL) at 20° C. and then the mixture was stirred at 20° C. for 1 hr. The mixture was concentrated under reduced pressure to give a residue. The residue was purified by preparative TLC eluting with 50% EtOAc in Pet. Ether to give 5-(3-aminophenyl)-N-(4-chloro-1H-indazol-5-yl)isoxazol-3-amine (20 mg, 61.40 umol, 45.75% yield) as a brown solid.

### Example 74: N-(3-(3-((4-chloro-1H-indazol-5-yl)amino)isoxazol-5-yl)phenyl)-1-methyl-1H-pyrazole-4-carboxamide

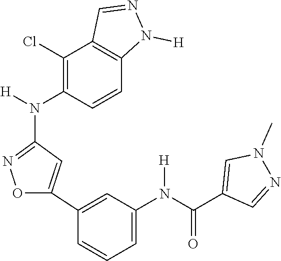 [SMILES: Cn1cc(C(=O)Nc2cccc(-c3cc(Nc4ccc5[nH]ncc5c4Cl)no3)c2)cn1]

To a solution of 5-(3-aminophenyl)-N-(4-chloro-1H-indazol-5-yl)isoxazol-3-amine (15 mg, 46.05 umol) in pyridine (0.1 mL) was added 1-methylpyrazole-4-carboxylic acid (11.61 mg, 92.09 umol), EDCI (17.65 mg, 92.09 umol) at 20° C. and the mixture was stirred at 20° C. for 16 hr. The mixture was concentrated under reduced pressure to give a residue. The residue was purified by preparative HPLC (25-55% MeCN in H₂O) to give N-(3-(3-((4-chloro-1H-indazol-5-yl)amino)isoxazol-5-yl)phenyl)-1-methyl-1H-pyrazole-4-carboxamide (3.7 mg, 7.20 umol, 15.63% yield, 93.37% purity, FA salt) as a white solid. LC-MS (ES⁺, Method A), 0.466 min, m/z 434.2. [M+H]⁺. ¹H NMR (400 MHz, MeOD-d₄) 6 8.21 (s, 1H), 8.12 (s, 1H), 8.07 (d, J=8.4 Hz, 2H), 7.78 (d, J=8.0 Hz, 1H), 7.65-7.50 (m, 3H), 7.49-7.39 (m, 1H), 5.70 (s, 1H), 3.97 (s, 3H).

General method for the synthesis of Example 75

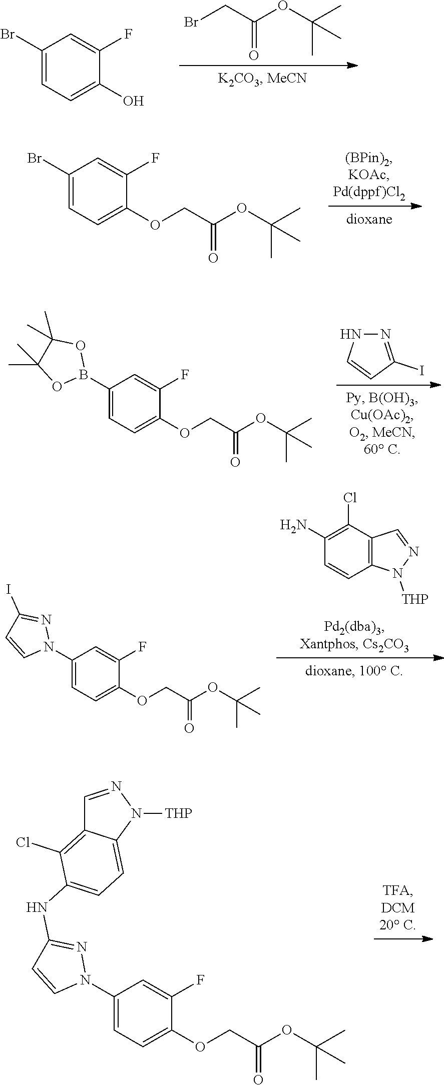

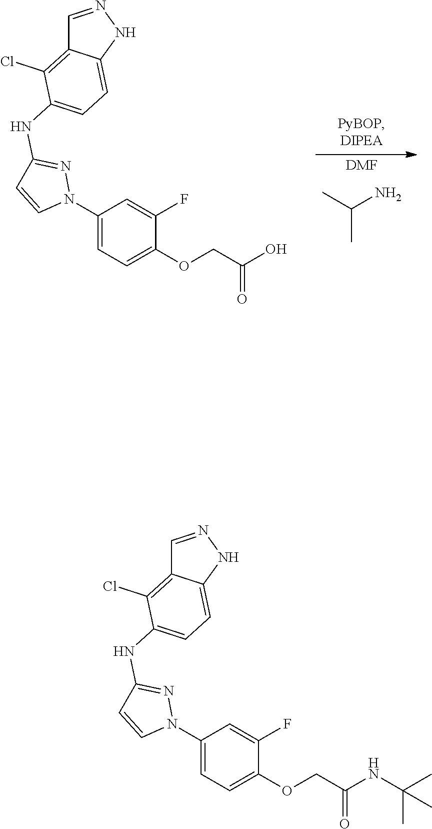

### Step 1: tert-butyl 2-(4-bromo-2-fluorophenoxy)acetate

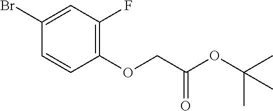 [SMILES: CC(C)(C)OC(=O)COc1ccc(Br)cc1F]

To a solution of 4-bromo-2-fluoro-phenol (10 g, 52.36 mmol), tert-butyl 2-bromoacetate (11.23 g, 57.59 mmol, 8.51 mL) in MeCN (100 mL) was added K₂CO₃ (14.47 g, 104.71 mmol). The mixture was stirred at 60° C. for 16 hr. The reaction mixture was concentrated under reduced pressure to remove solvent. The residue was diluted with water (100 mL) and extracted with ethyl acetate (30 mL×3). The combined organic layers were washed with brine (50 mL×2), dried over Na₂SO₄, filtered and concentrated under reduced pressure to give tert-butyl 2-(4-bromo-2-fluorophenoxy)acetate (15.9 g, 52.11 mmol, 99.52% yield) as a yellow oil.

### Step 2: tert-butyl 2-(2-fluoro-4-(4,4,5,5-tetramethyl-1,3,2-dioxaborolan-2-yl)phenoxy)acetate

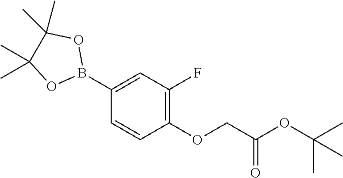 [SMILES: CC(C)(C)OC(=O)COc1ccc(B2OC(C)(C)C(C)(C)O2)cc1F]

To a mixture of tert-butyl 2-(4-bromo-2-fluorophenoxy)acetate (1 g, 3.28 mmol), 4,4,5,5-tetramethyl-2-(4,4,5,5-tetramethyl-1,3,2-dioxaborolan-2-yl)-1,3,2-dioxaborolane (915.42 mg, 3.60 mmol), KOAc (643.26 mg, 6.55 mmol), Pd(dppf)Cl₂ (239.79 mg, 327.72 umol) in dioxane (10 mL) was degassed and purged with nitrogen for 3 times, and then the mixture was stirred at 90° C. for 12 hr. The reaction mixture was poured into water (30 mL), extracted with ethyl acetate (20 mL×2). The combined organic layers were washed with brine (10 mL×2), dried over Na₂SO₄, filtered and concentrated. The residue was purified by column chromatography eluting with 0-10% EtOAc in Pet. Ether to give tert-butyl 2-[2-fluoro-4-(4,4,5,5-tetramethyl-1,3,2-dioxaborolan-2-yl)phenoxy]acetate (1.15 g, 3.27 mmol) as a yellow oil.

### Step 3: tert-butyl 2-(2-fluoro-4-(3-iodo-1H-pyrazol-1-yl)phenoxy)acetate

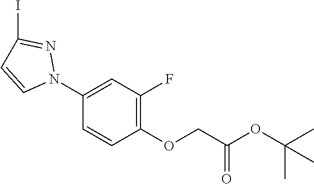 [SMILES: CC(C)(C)OC(=O)COc1ccc(-n2ccc(I)n2)cc1F]

To a solution of tert-butyl 2-[2-fluoro-4-(4,4,5,5-tetramethyl-1,3,2-dioxaborolan-2-yl)phenoxy]acetate (300 mg, 851.78 umol), 3-iodo-1H-pyrazole (165.22 mg, 851.78 umol) in MeCN (2 mL) was added pyridine (134.75 mg, 1.70 mmol), boric acid (105.34 mg, 1.70 mmol) and Cu(OAc)₂ (232.07 mg, 1.28 mmol). The mixture was stirred at 60° C. for 12 hr. The reaction mixture was filtered and the mother solution was concentrated, the residue was added into water (80 mL), extracted with ethyl acetate (50 mL×2). The combined organic layers were washed with brine (50 mL×2), dried over Na₂SO₄, filtered and concentrated under reduced pressure to give a residue. The residue was purified by column chromatography eluting with 0-25% EtOAc in Pet. Ether to give tert-butyl 2-[2-fluoro-4-(3-iodopyrazol-1-yl)phenoxy]acetate (240 mg, 573.89 umol, 67.38% yield) as a yellow solid.

### Step 4: tert-butyl 2-(4-(3-((4-chloro-1-(tetrahydro-2H-pyran-2-yl)-1H-indazol-5-yl)amino)-1H-pyrazol-1-yl)-2-fluorophenoxy)acetate

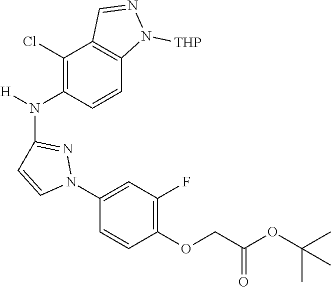

To a mixture of tert-butyl 2-[2-fluoro-4-(3-iodopyrazol-1-yl)phenoxy]acetate (240 mg, 573.89 umol), 4-chloro-1-tetrahydropyran-2-yl-indazol-5-amine (144.45 mg, 573.89 umol), Pd₂(dba)₃ (52.55 mg, 57.39 umol), Xantphos (99.62 mg, 172.17 umol) and Cs₂CO₃ (373.97 mg, 1.15 mmol) in dioxane (3 mL) was degassed and purged with nitrogen for 3 times, and then the mixture was stirred at 100° C. for 16 hr under nitrogen atmosphere. The reaction mixture was poured into water (30 mL), extracted with ethyl acetate (20 mL×2). The combined organic layers were washed with brine (10 mL×2), dried over Na₂SO₄, filtered and concentrated. The residue was purified by preparative HPLC (63-93% MeCN in H₂O) to give tert-butyl 2-(4-(3-((4-chloro-1-(tetrahydro-2H-pyran-2-yl)-1H-indazol-5-yl)amino)-1H-pyrazol-1-yl)-2-fluorophenoxy)acetate (55 mg, 101.48 umol, 17.68% yield) as a white solid. LC-MS (ES⁺, Method A), 0.662 min, m/z 542.3 [M+H]⁺.

### Step 5: 2-(4-(3-((4-chloro-1H-indazol-5-yl)amino)-1H-pyrazol-1-yl)-2-fluorophenoxy)acetic acid

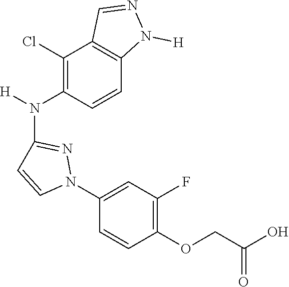 [SMILES: O=C(O)COc1ccc(-n2ccc(Nc3ccc4[nH]ncc4c3Cl)n2)cc1F]

To a solution of tert-butyl 2-(4-(3-((4-chloro-1-(tetrahydro-2H-pyran-2-yl)-1H-indazol-5-yl)amino)-1H-pyrazol-1-yl)-2-fluorophenoxy)acetate (55 mg, 101.48 umol) in DCM (0.5 mL) was added TFA (770.00 mg, 6.75 mmol, 0.5 mL). The mixture was stirred at 20° C. for 2 hr. The reaction mixture was concentrated under reduced pressure to give 2-(4-(3-((4-chloro-1H-indazol-5-yl)amino)-1H-pyrazol-1-yl)-2-fluorophenoxy)acetic acid (40 mg, 99.56 umol) as a yellow oil.

### Example 75: 2-(4-(3-((4-chloro-1H-indazol-5-yl)amino)-1H-pyrazol-1-yl)-2-fluorophenoxy)—N-isopropylacetamide

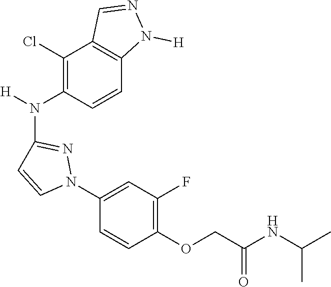 [SMILES: CC(C)NC(=O)COc1ccc(-n2ccc(Nc3ccc4[nH]ncc4c3Cl)n2)cc1F]

To a solution of 2-(4-(3-((4-chloro-1H-indazol-5-yl)amino)-1H-pyrazol-1-yl)-2-fluorophenoxy)acetic acid (40 mg, 99.56 umol), propan-2-amine (5.88 mg, 99.56 umol) in DMF (1 mL) was added PyBOP (103.62 mg, 199.11 umol) and DIEA (25.73 mg, 199.11 umol). The mixture was stirred at 25° C. for 2 hr. The reaction mixture was purified by preparative HPLC (30-60% MeCN in H₂O) to give 2-[4-[3-[(4-chloro-1H-indazol-5-yl)amino]pyrazol-1-yl]-2-fluoro-phenoxy]-N-isopropyl-acetamide (9.5 mg, 20.96 umol, 21.05% yield) as an off-white solid. LC-MS (ES⁺, Method A), 0.514 min, m/z 443.2 [M+H]⁺. ¹H NMR (400 MHz, DMSO-d₆) δ 13.20 (d, J=2.0 Hz, 1H), 8.29 (s, 1H), 8.20-8.12 (m, 1H), 8.07-7.96 (m, 2H), 7.90 (d, J=0.8 Hz, 1H), 7.67 (d, J=11.6 Hz, 1H), 7.51 (s, 2H), 7.15 (d, J=1.2 Hz, 1H), 6.20 (s, 1H), 4.54 (s, 2H), 4.01-3.87 (m, 1H), 1.09 (s, 6H).

General method for the synthesis of Example 76

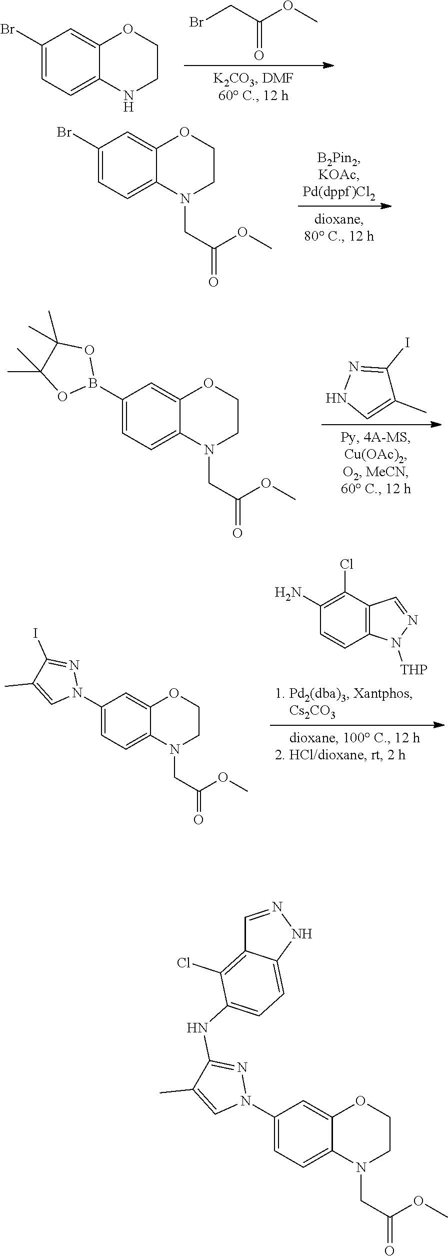

### Step 1: methyl 2-(7-bromo-2,3-dihydro-1,4-benzoxazin-4-yl)acetate

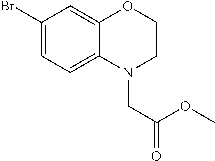 [SMILES: COC(=O)CN1CCOc2cc(Br)ccc21]

To a solution of 7-bromo-3,4-dihydro-2H-1,4-benzoxazine (2 g, 9.34 mmol), methyl 2-bromoacetate (2.14 g, 14.01 mmol, 1.32 mL) in MeCN (20 mL) was added K₂CO₃ (2.58 g, 18.69 mmol). The mixture was stirred at 60° C. for 12 hr. The reaction mixture was concentrated under reduced pressure. The residue was purified by flash silica gel chromatography eluting with 0-25% EtOAc in Pet. Ether to give methyl 2-(7-bromo-2,3-dihydro-1,4-benzoxazin-4-yl)acetate (5.3 g, 18.52 mmol, 99.13% yield) as a brown oil. LC-MS (ES⁺, Method A), 0.46 min, m/z 285.5 [M+H]⁺.

### Step 2: methyl 2-[7-(4,4,5,5-tetramethyl-1,3,2-dioxaborolan-2-yl)-2,3-dihydro-1,4-benzoxazin-4-yl]acetate

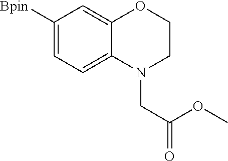

A mixture of methyl 2-(7-bromo-2,3-dihydro-1,4-benzoxazin-4-yl)acetate (1 g, 3.50 mmol), 4,4,5,5-tetramethyl-2-(4,4,5,5-tetramethyl-1,3,2-dioxaborolan-2-yl)-1,3,2-dioxaborolane (1.33 g, 5.24 mmol), Pd(dppf)Cl₂.CH₂Cl₂ (285.42 mg, 349.50 umol), KOAc (686.02 mg, mmol, 2 eq) in dioxane (10 mL) was degassed and purged with N₂ for 3 times, and then the mixture was stirred at 80° C. for 16 hr under N₂ atmosphere. The reaction mixture was concentrated under reduced pressure to give a residue. The residue was purified by flash silica gel chromatography eluting with 0-25% EtOAc in Pet. Ether to give methyl 2-[7-(4,4,5,5-tetramethyl-1,3,2-dioxaborolan-2-yl)-2,3-dihydro-1,4-benzoxazin-4-yl]acetate (2.3 g, 6.90 mmol, 98.76% yield) as a yellow solid. LC-MS (ES⁺, Method A), 0.48 min, m/z 333.6 [M+H]⁺.

### Step 3: methyl 2-[7-(3-iodo-4-methyl-pyrazol-1-yl)-2,3-dihydro-1,4-benzoxazin-4-yl]acetate

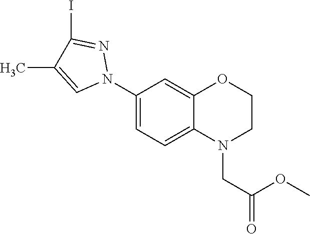

To a solution of methyl 2-[7-(4,4,5,5-tetramethyl-1,3,2-dioxaborolan-2-yl)-2,3-dihydro-1,4-benzoxazin-4-yl]acetate (1 g, 3.00 mmol), 3-iodo-4-methyl-1H-pyrazole (561.85 mg, 2.70 mmol) in MeCN (10 mL) was added Py (356.11 mg, 4.50 mmol, 363.37 uL) and Cu(OAc)₂ (1.09 g, 6.00 mmol), 4 Å MS (500 mg, 3.00 mmol) and boric acid (371.16 mg, 6.00 mmol). The mixture was stirred at 60° C. for 12 hr under O₂ (15 psi). The reaction mixture was concentrated under reduced pressure to give a residue. The residue was purified by flash silica gel chromatography eluting with 0-25% EtOAc in Pet. Ether to give and preparative HPLC (50-80% MeCN in H₂O) to give methyl 2-[7-(3-iodo-4-methyl-pyrazol-1-yl)-2,3-dihydro-1,4-benzoxazin-4-yl]acetate (220 mg, 532.42 umol, 17.74% yield) as a yellow solid). LC-MS (ES⁺, Method A), 0.50 min, m/z 413.5 [M+H]⁺.

### Step 4: methyl 2-[7-[3-[(4-chloro-1-tetrahydropyran-2-yl-indazol-5-yl)amino]-4-methyl-pyrazol-1-yl]-2,3-dihydro-1,4-benzoxazin-4-yl]acetate

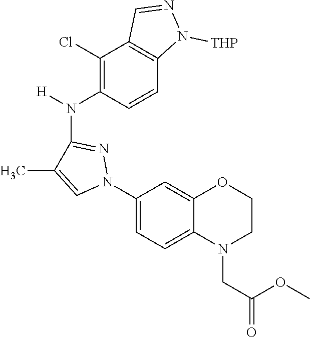

A mixture of methyl 2-[7-(3-iodo-4-methyl-pyrazol-1-yl)-2,3-dihydro-1,4-benzoxazin-4-yl]acetate (200 mg, 484.02 umol, 1 eq), 4-chloro-1-tetrahydropyran-2-yl-indazol-5-amine (121.83 mg, 484.02 umol, 1 eq), Pd₂(dba)₃ (22.16 mg, 24.20 umol), Xantphos (28.01 mg, 48.40 umol) and Cs₂CO₃ (315.40 mg, 968.03 umol) in dioxane (2 mL) was degassed and purged with N₂ for 3 times, and then the mixture was stirred at 100° C. for 16 hr under N₂ atmosphere. The reaction mixture was concentrated under reduced pressure to give a residue. The residue was purified by preparative TLC eluting with 30% EtOAc in Pet. Ether to give methyl 2-[7-[3-[(4-chloro-1-tetrahydropyran-2-yl-indazol-5-yl)amino]-4-methyl-pyrazol-1-yl]-2,3-dihydro-1,4-benzoxazin-4-yl]acetate (50 mg, 93.11 umol, 19.24% yield) as a brown solid. LC-MS (ES⁺, Method A), 0.58 min, m/z 537.1 [M+H]⁺.

### Example 76: methyl 2-[7-[3-[(4-chloro-1H-indazol-5-yl)amino]-4-methyl-pyrazol-1-yl]-2,3-dihydro-1,4-benzoxazin-4-yl]acetate

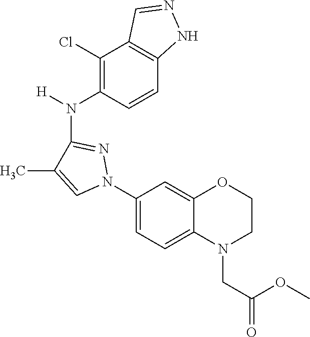

A mixture of methyl 2-[7-[3-[(4-chloro-1-tetrahydropyran-2-yl-indazol-5-yl)amino]-4-methyl-pyrazol-1-yl]-2,3-dihydro-1,4-benzoxazin-4-yl]acetate (25 mg, 46.55 umol, 1 eq) in HCl/dioxane (1 mL) was stirred at 25° C. for 0.5 hr. The reaction mixture was concentrated under reduced pressure to give a residue. The residue was purified by preparative HPLC (50-80% MeCN in H₂O) to give methyl 2-[7-[3-[(4-chloro-1H-indazol-5-yl)amino]-4-methyl-pyrazol-1-yl]-2,3-dihydro-1,4-benzoxazin-4-yl]acetate (19.2 mg, 41.89 umol, 89.98% yield, 98.805% purity) as an off-white solid. LC-MS (ES⁺, Method A), 0.50 min, m/z 452.9 [M+H]⁺. ¹H NMR (400 MHz, DMSO-d₆) δ 13.16 (s, 1H), 8.04 (s, 1H), 7.97 (s, 1H), 7.64 (d, J=8.8 Hz, 1H), 7.44 (d, J=8.8 Hz, 1H), 7.10-7.01 (m, 3H), 6.65-6.57 (m, 1H), 4.26-4.19 (m, 4H), 3.65 (s, 3H), 3.46-3.40 (m, 2H), 1.97 (s, 3H).

General method for the synthesis of Example 77

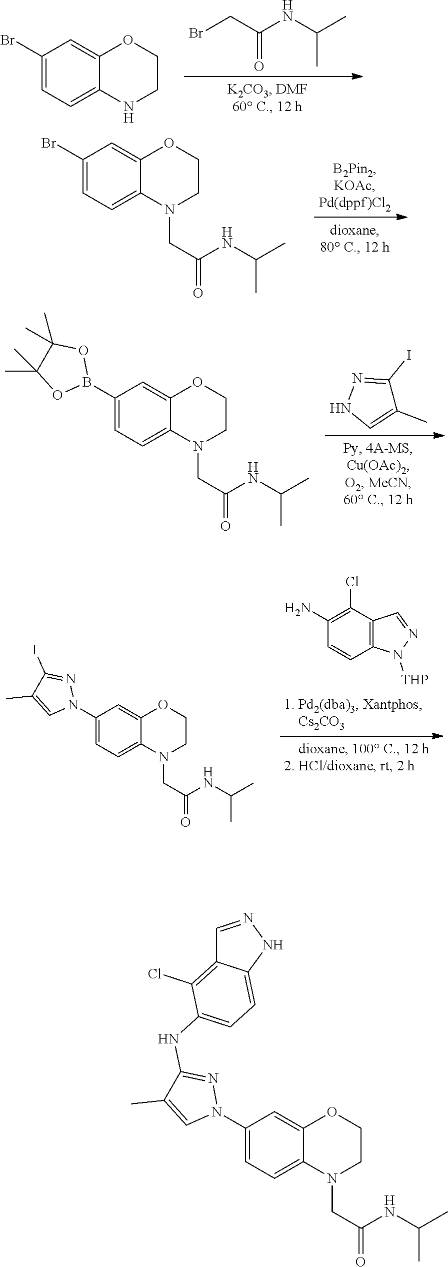

### Step 1: 2-(7-bromo-2,3-dihydro-1,4-benzoxazin-4-yl)-N-isopropyl-acetamide

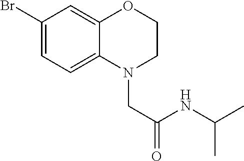 [SMILES: CC(C)NC(=O)CN1CCOc2cc(Br)ccc21]

To a solution of 7-bromo-3,4-dihydro-2H-1,4-benzoxazine (2 g, 9.34 mmol), 2-bromo-N-isopropyl-acetamide (2.52 g, 14.01 mmol) in MeCN (10 mL) was added K₂CO₃ (2.58 g, 18.69 mmol). The mixture was stirred at 60° C. for 2 hr. The reaction mixture was filtered and concentrated under reduced pressure to give a residue. The residue was purified by flash silica gel chromatography eluting with 0-50% EtOAc in Pet. Ether to give 2-(7-bromo-2,3-dihydro-1,4-benzoxazin-4-yl)-N-isopropyl-acetamide (2 g, 6.39 mmol, 68.35% yield) as a white solid. LC-MS (ES⁺, Method A), 0.42 min, m/z 315.0 [M+H]⁺.

### Step 2: N-isopropyl-2-[7-(4,4,5,5-tetramethyl-1,3,2-dioxaborolan-2-yl)-2,3-dihydro-1,4-benzoxazin-4-yl]acetamide

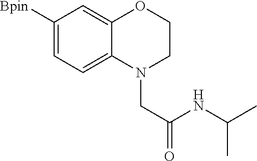

A mixture of 2-(7-bromo-2,3-dihydro-1,4-benzoxazin-4-yl)-N-isopropyl-acetamide (2 g, 6.39 mmol), 4,4,5,5-tetramethyl-2-(4,4,5,5-tetramethyl-1,3,2-dioxaborolan-2-yl)-1,3,2-dioxaborolane (2.43 g, 9.58 mmol), KOAc (1.25 g, 12.77 mmol), Pd(dppf)Cl₂.CH₂Cl₂ (521.50 mg, 638.59 umol) in dioxane (5 mL) was degassed and purged with N₂ for 3 times, and then the mixture was stirred at 100° C. for 12 hr under N₂ atmosphere. The reaction mixture was concentrated under reduced pressure to give a residue. The residue was purified by flash silica gel chromatography eluting with 0-50% EtOAc in Pet. Ether to give N-isopropyl-2-[7-(4,4,5,5-tetramethyl-1,3,2-dioxaborolan-2-yl)-2,3-dihydro-1,4-benzoxazin-4-yl]acetamide (2 g, 5.55 mmol, 86.94% yield) as a yellow oil. LC-MS (ES⁺, Method A), 0.44 min, m/z 361.0 [M+H]⁺.

### Step 3: 2-[7-(3-iodo-4-methyl-pyrazol-1-yl)-2,3-dihydro-1,4-benzoxazin-4-yl]-N-isopropyl-acetamide

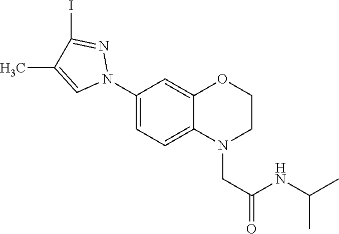 [SMILES: Cc1cn(-c2ccc3c(c2)OCCN3CC(=O)NC(C)C)nc1I]

To a solution of N-isopropyl-2-[7-(4,4,5,5-tetramethyl-1,3,2-dioxaborolan-2-yl)-2,3-dihydro-1,4-benzoxazin-4-yl]acetamide (2 g, 5.55 mmol), 3-iodo-4-methyl-1H-pyrazole (1.04 g, 5.00 mmol) in MeCN (20 mL) was added Cu(OAc)₂ (2.02 g, 11.10 mmol) and pyridine (658.70 mg, 8.33 mmol, 672.14 uL), 4A MS (1 g, 5.55 mmol), boric acid (686.51 mg, 11.10 mmol). The mixture was stirred at 60° C. for 16 hr under O₂ (15 psi). The reaction mixture was filtered and concentrated under reduced pressure to give a residue. The residue was purified by flash silica gel chromatography eluting with 0-75% EtOAc in Pet. Ether and preparative HPLC (10-20% MeCN in H₂O) to give 2-[7-(3-iodo-4-methyl-pyrazol-1-yl)-2,3-dihydro-1,4-benzoxazin-4-yl]-N-isopropyl-acetamide (330 mg, 749.53 umol, 13.50% yield) as a white solid. LC-MS (ES⁺, Method A), 0.49 min, m/z 440.7 [M+H]⁺.

### Step 4: 2-[7-[3-[(4-chloro-1-tetrahydropyran-2-yl-indazol-5-yl)amino]-4-methyl-pyrazol-1-yl]-2,3-dihydro-1,4-benzoxazin-4-yl]-N-isopropyl-acetamide

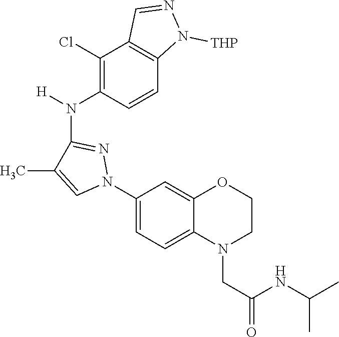

A mixture of 2-[7-(3-iodo-4-methyl-pyrazol-1-yl)-2,3-dihydro-1,4-benzoxazin-4-yl]-N-isopropyl-acetamide (100 mg, 227.13 umol), 4-chloro-1-tetrahydropyran-2-yl-indazol-5-amine (57.17 mg, 227.13 umol), 4-chloro-1-tetrahydropyran-2-yl-indazol-5-amine (57.17 mg, 227.13 umol), Pd₂(dba)₃ (20.80 mg, 22.71 umol), Xantphos (13.14 mg, 22.71 umol) and Cs₂CO₃ (148.01 mg, 454.26 umol) in dioxane (0.5 mL) was degassed and purged with N₂ for 3 times, and then the mixture was stirred at 100° C. for 12 hr under N₂ atmosphere. The reaction mixture was filtered and concentrated under reduced pressure to give a residue. The residue was purified by preparative TLC eluting with 0-60% EtOAc in Pet. Ether to give 2-[7-[3-[(4-chloro-1-tetrahydropyran-2-yl-indazol-5-yl)amino]-4-methyl-pyrazol-1-yl]-2,3-dihydro-1,4-benzoxazin-4-yl]-N-isopropyl-acetamide (60 mg, 106.37 umol, 46.83% yield) as a yellow solid. LC-MS (ES⁺, Method A), 0.54 min, m/z 564.2 [M+H]⁺.

### Example 77: 2-[7-[3-[(4-chloro-1H-indazol-5-yl)amino]-4-methyl-pyrazol-1-yl]-2,3-dihydro-1,4-benzoxazin-4-yl]-N-isopropyl-acetamide

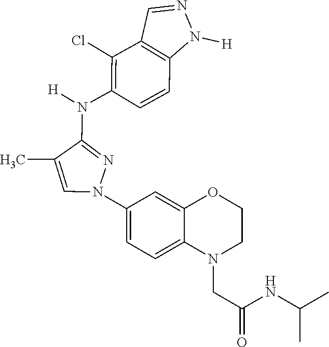

To a solution of 2-[7-[3-[(4-chloro-1-tetrahydropyran-2-yl-indazol-5-yl)amino]-4-methyl-pyrazol-1-yl]-2,3-dihydro-1,4-benzoxazin-4-yl]-N-isopropyl-acetamide (60 mg, 106.37 umol) in HCl/dioxane (2 mL), DCM (2 mL) was stirred at 25° C. for 0.5 hr. The reaction mixture was concentrated under reduced pressure to give a residue. The residue was purified by preparative HPLC (35-65% MeCN in H₂O) to give 2-[7-[3-[(4-chloro-1H-indazol-5-yl)amino]-4-methyl-pyrazol-1-yl]-2,3-dihydro-1,4-benzoxazin-4-yl]-N-isopropyl-acetamide (18.3 mg, 36.98 umol, 34.76% yield, 96.977% purity) as a white solid. LC-MS (ES⁺, Method A), 0.48 min, m/z 479.9 [M+H]⁺. ¹H NMR (400 MHz, DMSO-d₆) δ 13.16 (s, 1H), 8.18 (s, 1H), 8.03 (s, 1H), 7.96 (s, 1H), 7.84 (d, J=8.0 Hz, 1H), 7.60 (d, J=8.8 Hz, 1H), 7.44 (d, J=8.8 Hz, 1H), 7.06 (d, J=7.6 Hz, 3H), 6.53 (d, J=9.2 Hz, 1H), 4.25 (s, 2H), 3.92-3.84 (m, 1H), 3.81 (s, 2H), 3.43 (s, 2H), 1.96 (s, 3H), 1.05 (d, J=6.4 Hz, 6H).

### General Route or the Synthesis of Examples 78-82

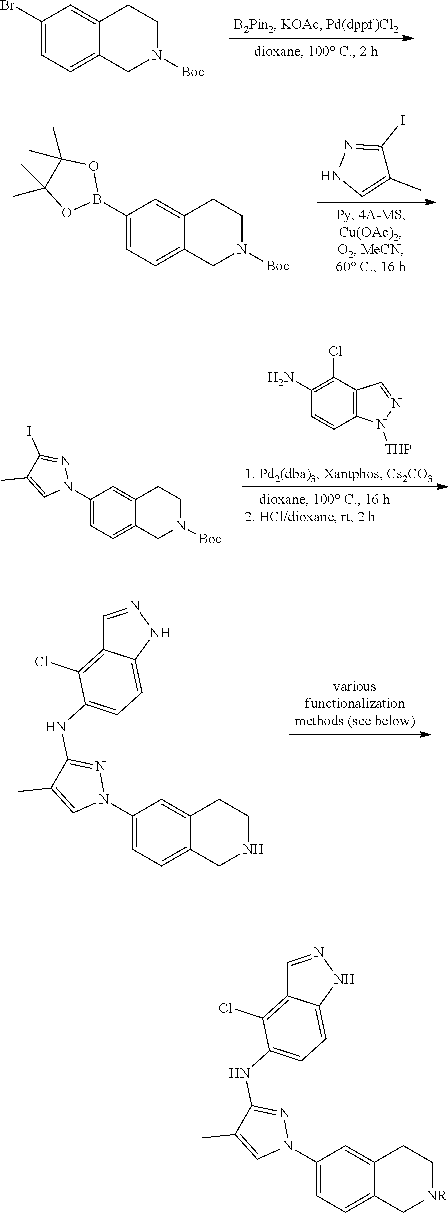

### Step 1: tert-butyl 6-(4,4,5,5-tetramethyl-1,3,2-dioxaborolan-2-yl)-3,4-dihydro-1H-isoquinoline-2-carboxylate

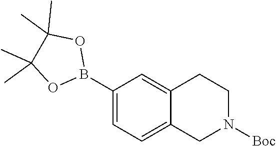 [SMILES: CC(C)(C)OC(=O)N1CCc2cc(B3OC(C)(C)C(C)(C)O3)ccc2C1]

A mixture of tert-butyl 6-bromo-3,4-dihydro-1H-isoquinoline-2-carboxylate (5 g, 16.02 mmol), 4,4,5,5-tetramethyl-2-(4,4,5,5-tetramethyl-1,3,2-dioxaborolan-2-yl)-1,3,2-dioxaborolane (6.10 g, 24.02 mmol), Pd(dppf)Cl₂·CH₂Cl₂ (1.31 g, 1.60 mmol), KOAc (3.14 g, 32.03 mmol) in dioxane (50 mL) was degassed and purged with N₂ for 3 times, and then the mixture was stirred at 100° C. for 16 hr under N₂ atmosphere. The reaction mixture was concentrated under reduced pressure to give a residue. The residue was purified by flash silica gel chromatography eluting with 0-10% EtOAc in Pet. Ether to give tert-butyl 6-(4,4,5,5-tetramethyl-1,3,2-dioxaborolan-2-yl)-3,4-dihydro-1H-isoquinoline-2-carboxylate (3.3 g, 9.19 mmol, 57.35% yield) as a yellow solid. LC-MS (ES⁺, Method A), 0.79 min, m/z 385.4 [M+H]⁺.

### Step 2: tert-butyl 6-(3-iodo-4-methyl-pyrazol-1-yl)-3,4-dihydro-1H-isoquinoline-2-carboxylate

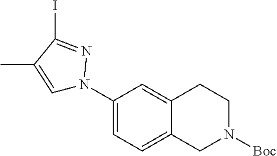 [SMILES: Cc1cn(-c2ccc3c(c2)CCN(C(=O)OC(C)(C)C)C3)nc1I]

To a solution of tert-butyl 6-(4,4,5,5-tetramethyl-1,3,2-dioxaborolan-2-yl)-3,4-dihydro-1H-isoquinoline-2-carboxylate (3 g, 8.35 mmol), 3-iodo-4-methyl-1H-pyrazole (1.74 g, 8.35 mmol) in MeCN (2 mL) was added Cu(OAc)₂ (2.28 g, 12.53 mmol) and 4 Å MS (1.5 g), pyridine (1.32 g, 16.70 mmol, 1.35 mL), boric acid (1.03 g, 16.70 mmol). The mixture was stirred at 60° C. for 16 hr under O₂ (15 Psi). The reaction mixture was filtered and concentrated under reduced pressure to remove 4A MS. The residue was concentrated under reduced pressure to give a residue. The residue was purified by flash silica gel chromatography eluting with 0-23% EtOAc in Pet. Ether to give tert-butyl 6-(3-iodo-4-methyl-pyrazol-1-yl)-3,4-dihydro-1H-isoquinoline-2-carboxylate (1.8 g, 4.10 mmol, 49.07% yield) as a colorless oil. LC-MS (ES⁺, Method A), 0.61 min, m/z 439.9 [M+H]⁺.

### Step 3: tert-butyl 6-[3-[(4-chloro-1-tetrahydropyran-2-yl-indazol-5-yl)amino]-4-methyl-pyrazol-1-yl]-3,4-dihydro-1H-isoquinoline-2-carboxylate

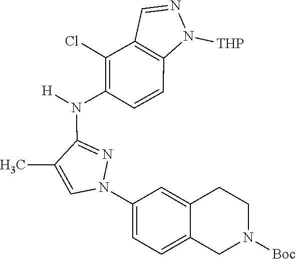

A mixture of tert-butyl 6-(3-iodo-4-methyl-pyrazol-1-yl)-3,4-dihydro-1H-isoquinoline-2-carboxylate (500 mg, 1.14 mmol), 4-chloro-1-tetrahydropyran-2-yl-indazol-5-amine (257.85 mg, 1.02 mmol), Pd₂(dba)₃ (52.11 mg, 56.91 umol), Xantphos (65.86 mg, 113.82 umol) and Cs₂CO₃ (741.70 mg, 2.28 mmol) in dioxane (5 mL) was degassed and purged with N₂ for 3 times, and then the mixture was stirred at 100° C. for 16 hr under N₂ atmosphere. The reaction mixture was concentrated under reduced pressure to give a residue. The residue was purified by flash silica gel chromatography eluting with 0-23% EtOAc in Pet. Ether and preparative HPLC (80-100% MeCN in H₂O) to give tert-butyl 6-[3-[(4-chloro-1-tetrahydropyran-2-yl-indazol-5-yl)amino]-4-methyl-pyrazol-1-yl]-3,4-dihydro-1H-isoquinoline-2-carboxylate (400 mg, 710.37 umol, 31.21% yield) as a white solid. LC-MS (ES⁺, Method A), 0.57 min, m/z 563.2 [M+H]⁺.

### Step 4: 4-chloro-N-[4-methyl-1-(1,2,3,4-tetrahydroisoquinolin-6-yl)pyrazol-3-yl]-1H-indazol-5-amine

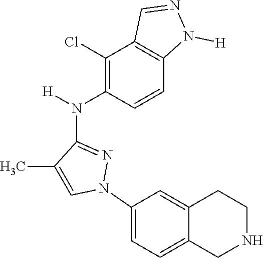 [SMILES: Cc1cn(-c2ccc3c(c2)CCNC3)nc1Nc1ccc2[nH]ncc2c1Cl]

A mixture of tert-butyl 6-[3-[(4-chloro-1-tetrahydropyran-2-yl-indazol-5-yl)amino]-4-methyl-pyrazol-1-yl]-3,4-dihydro-1H-isoquinoline-2-carboxylate (290 mg, 515.02 umol) in HCl/dioxane (5 mL) was stirred at 25° C. for 1 hr. The reaction mixture was concentrated under reduced pressure to give 4-chloro-N-[4-methyl-1-(1,2,3,4-tetrahydroisoquinolin-6-yl)pyrazol-3-yl]-1H-indazol-5-amine (250 mg, HCl salt) as yellow solid. LC-MS (ES⁺, Method A), 0.48 min, m/z 379.0 [M+H]⁺.

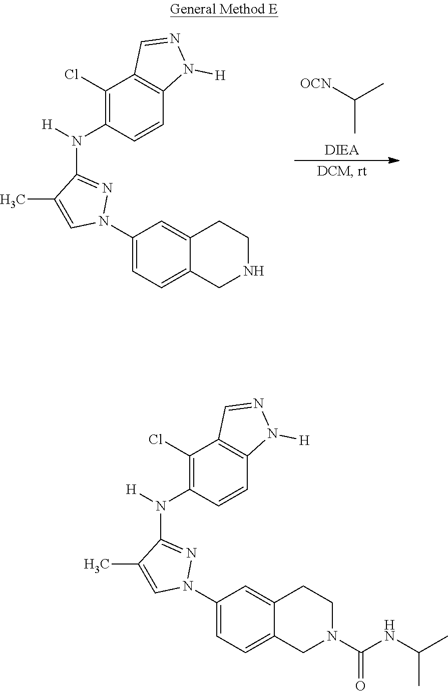

### Example 78: 6-(3-((4-chloro-1H-indazol-5-yl)amino)-4-methyl-1H-pyrazol-1-yl)-N-isopropyl-3,4-dihydroisoquinoline-2(1H)-carboxamide

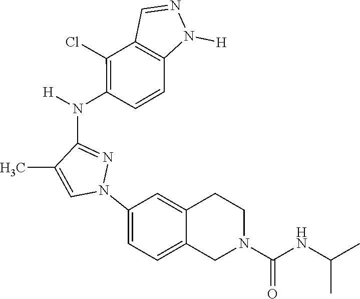 [SMILES: Cc1cn(-c2ccc3c(c2)CCN(C(=O)NC(C)C)C3)nc1Nc1ccc2[nH]ncc2c1Cl]

To a solution of 4-chloro-N-(4-methyl-1-(1,2,3,4-tetrahydroisoquinolin-6-yl)-1H-pyrazol-3-yl)-1H-indazol-5-amine (170 mg, 448.72 umol) and 2-isocyanatopropane (381.88 mg, 4.49 mmol, 439.95 uL) in DCM (2 mL) was added DIEA (173.98 mg, 1.35 mmol, 234.48 uL). The mixture was stirred at 25° C. for 2 hr. The residue was purified by preparative HPLC (40-70% MeCN in H₂O) to give phenyl (6-[3-[(4-chloro-1H-indazol-5-yl)amino]-4-methyl-pyrazol-1-yl]-N-isopropyl-3,4-dihydro-1H-isoquinoline-2-carboxamide (30 mg, 64.66 umol, 14.41% yield) as a white solid. LC-MS (ES⁺, Method A), 0.585 min, m/z 464.2 [M+H]⁺. ¹H NMR (400 MHz, DMSO-d₆) δ 13.19 (s, 1H), 8.19 (s, 1H), 7.99 (s, 1H), 7.69 (d, J=8.8 Hz, 1H), 7.52-7.46 (m, 2H), 7.19-7.16 (m, 2H), 6.22 (d, J=7.2 Hz, 1H), 4.47 (s, 2H), 3.86-3.70 (m, 1H), 3.55 (t, J=6.0 Hz, 2H), 2.80 (t, J=5.6 Hz, 2H), 2.01 (s, 4H), 1.08 (d, J=6.8 Hz, 6H).

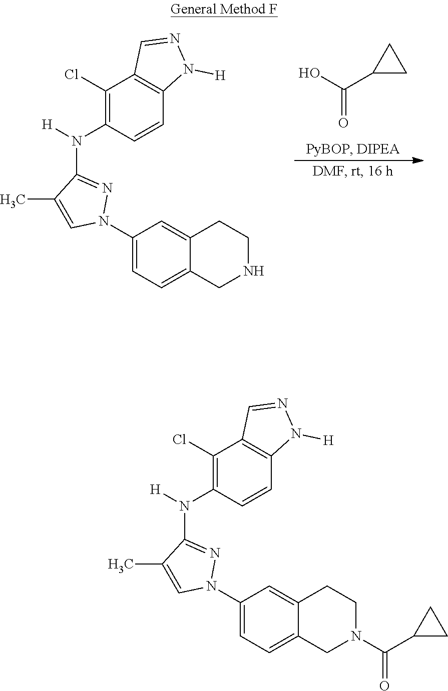

### Example 79: [6-[3-[(4-chloro-1H-indazol-5-yl)amino]-4-methyl-pyrazol-1-yl]-3,4-dihydro-1H-isoquinolin-2-yl]-cyclopropyl-methanone

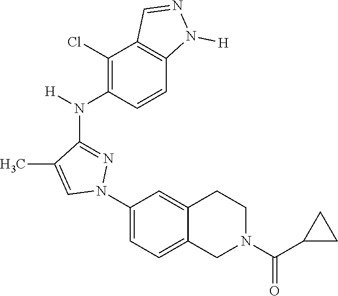 [SMILES: Cc1cn(-c2ccc3c(c2)CCN(C(=O)C2CC2)C3)nc1Nc1ccc2[nH]ncc2c1Cl]

To a solution of 4-chloro-N-[4-methyl-1-(1,2,3,4-tetrahydroisoquinolin-6-yl)pyrazol-3-yl]-1H-indazol-5-amine (30 mg, 72.23 umol, HCl salt), cyclopropanecarboxylic acid (6.22 mg, 72.23 umol, 5.71 uL) in DMF (0.5 mL) was added PyBOP (75.18 mg, 144.47 umol) and DIEA (46.68 mg, 361.17 umol, 62.91 uL). The mixture was stirred at 25° C. for 2 hr. The reaction mixture was diluted with water (10 mL) and extracted with EtOAc (10 mL×3). The combined organic layers were dried over Na₂SO₄, filtered and concentrated under reduced pressure to give a residue. And then to a solution of the crude in MeOH (1 mL) was added K₂CO₃ (20 mg). The mixture was stirred at 25° C. for 2 hr. The reaction mixture was filtered and concentrated under reduced pressure to give a residue after 2 hr. The residue was purified by preparative HPLC (48-78% MeCN in H₂O) to give [6-[3-[(4-chloro-1H-indazol-5-yl)amino]-4-methyl-pyrazol-1-yl]-3,4-dihydro-1H-isoquinolin-2-yl]-cyclopropyl-methanone (9.4 mg, 20.13 umol, 27.87% yield) as an off-white solid. LC-MS (ES⁺, Method A), 0.48 min, m/z 447.0 [M+H]⁺.¹HNMR (400 MHz, DMSO-d₆) δ 13.18 (s, 1H), 8.20 (s, 1H), 7.99 (s, 1H), 7.69 (d, J=8.8 Hz, 1H), 7.53 (s, 2H), 7.46 (d, J=9.2 Hz, 1H), 7.29-7.23 (m, 1H), 7.17 (s, 1H), 4.88 (s, 1H), 4.59 (s, 1H), 3.91 (s, 1H), 3.69 (s, 1H), 2.94 (s, 1H), 2.80 (s, 1H), 2.10-2.03 (m, 1H), 2.00 (s, 3H), 0.78-0.71 (m, 4H).

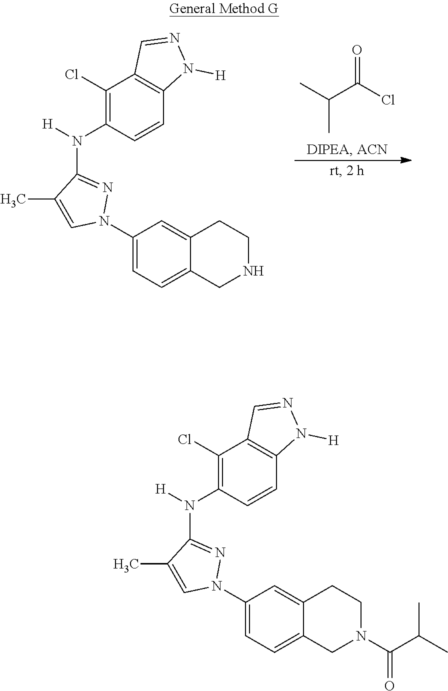

### Example 80: 1-[6-[3-[(4-chloro-1H-indazol-5-yl)amino]-4-methyl-pyrazol-1-yl]-3,4-dihydro-1H-isoquinolin-2-yl]-2-methyl-propan-1-one

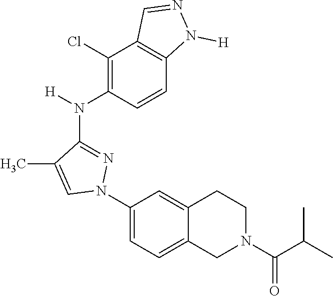 [SMILES: Cc1cn(-c2ccc3c(c2)CCN(C(=O)C(C)C)C3)nc1Nc1ccc2[nH]ncc2c1Cl]

To a solution of 4-chloro-N-[4-methyl-1-(1,2,3,4-tetrahydroisoquinolin-6-yl)pyrazol-3-yl]-1H-indazol-5-amine (30 mg, 72.23 umol, HCl salt), 2-methylpropanoyl chloride (7.70 mg, 72.23 umol, 7.55 uL) in MeCN (0.5 mL) was added DIEA (46.68 mg, 361.17 umol, 62.91 uL). The mixture was stirred at 25° C. for 1 hr. The reaction mixture was diluted with water (10 mL) and extracted with EtOAc (10 mL×3). The combined organic layers were dried over Na₂SO₄, filtered and concentrated under reduced pressure to give a residue. And then to a solution of the residue in MeOH (1 mL) was added K₂CO₃ (20 mg). The mixture was stirred at 25° C. for 2 hr. The reaction mixture was filtered and concentrated under reduced pressure to give a residue after 2 hr. The residue was purified by preparative HPLC (50-80% MeCN in H₂O) to give 1-[6-[3-[(4-chloro-1H-indazol-5-yl)amino]-4-methyl-pyrazol-1-yl]-3,4-dihydro-1H-isoquinolin-2-yl]-2-methyl-propan-1-one (10.4 mg, 22.82 umol, 31.59% yield, 98.501% purity) as an off-white solid. LC-MS (ES⁺, Method A), 0.49 min, m/z 448.9 [M+H]⁺. ¹H NMR (400 MHz, DMSO-d₆) δ 13.19 (s, 1H), 8.20 (s, 1H), 7.99 (s, 1H), 7.69 (d, J=9.2 Hz, 1H), 7.57-7.38 (m, 3H), 7.31-7.23 (m, 1H), 7.18 (s, 1H), 4.70 (s, 1H), 4.60 (s, 1H), 3.80-3.63 (m, 2H), 3.02-2.87 (m, 2H), 2.80 (s, 1H), 2.01 (s, 3H), 1.11-0.95 (m, 6H).

Compounds prepared in a similar manner to that set out above are given below in Table 19

TABLE 19

| Example | Structure | Method | LCMS | 1H NMR |
|---|---|---|---|---|
| 81 | 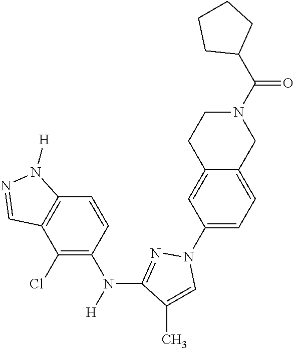 [SMILES: Cc1cn(-c2ccc3c(c2)CCN(C(=O)C2CCCC2)C3)nc1Nc1ccc2[nH]ncc2c1Cl] | F | LC-MS (ES+, Method A), 0.52 min, m/z 475.1 [M + H]+. | 1H NMR (400 MHz, DMSO-d6) δ 13.19 (s, 1 H), 8.20 (s, 1 H), 7.99 (s, 1 H), 7.69 (d, J = 8.8 Hz, 1 H), 7.56- 7.41 (m, 3 H), 7.25 (t, J = 7.2 Hz, 1 H), 7.18 (s, 1 H), 4.70 (s, 1 H), 4.60 (s, 1 H), 3.75 (t, J = 5.2 Hz, 1 H), 3.71-3.68 (m, 1 H), 3.13-3.04 (m, 1 H), 2.94- 2.86 (m, 1 H), 2.81-2.79 (m, 1 H), 2.01 (s, 3 H), 1.85-1.75 (m, 2 H), 1.72- 1.48 (m, 6 H). |
| 82 | 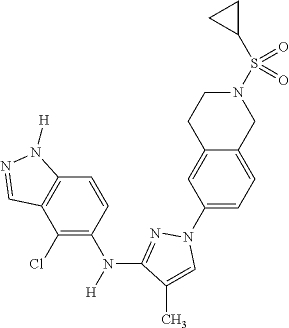 [SMILES: Cc1cn(-c2ccc3c(c2)CCN(S(=O)(=O)C2CC2)C3)nc1Nc1ccc2[nH]ncc2c1Cl] | G | LC-MS (ES+, Method A), 0.57 min, m/z 483.2 [M + H]+. | 1H NMR (400 MHz, DMSO-d6) δ 13.20 (s, 1 H), 8.21 (s, 1 H), 7.99 (s, 1 H), 7.70 (d, J = 9.2 Hz, 1 H), 7.57- 7.50 (m, 2 H), 7.47 (d, J = 8.4 Hz, 1 H), 7.25 (d, J = 8.4 Hz, 1 H), 7.19 (s, 1 H), 4.43 (s, 2H), 3.53 (t, J = 6.0 Hz, 2 H), 2.96 (t, J = 5.6 Hz, 2 H), 2.70-2.59 (m, 1 H), 2.01 (s, 3 H), 1.02-0.93 (m, 4 H). |

Method for the synthesis of Example 83

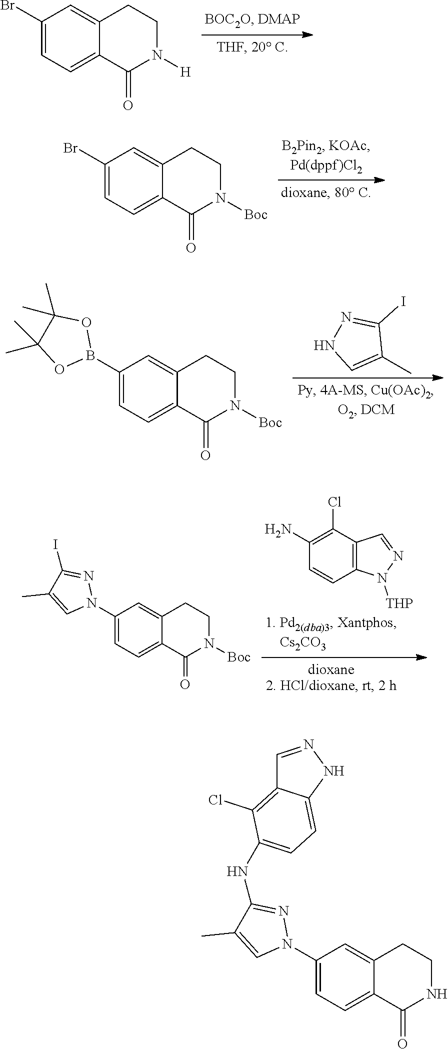

### Step 1: tert-butyl 6-bromo-1-oxo-3,4-dihydroisoquinoline-2(1H)-carboxylate

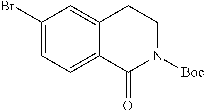 [SMILES: CC(C)(C)OC(=O)N1CCc2cc(Br)ccc2C1=O]

To a solution of 6-bromo-3,4-dihydroisoquinolin-1(2H)-one (3 g, 13.27 mmol) in THF (30 mL) was added Boc₂O (3.19 g, 14.60 mmol) and DMAP (243.18 mg, 1.99 mmol), the mixture was stirred at 25° C. for 1 hr. The reaction mixture was poured into water (100 mL), extracted with Ethyl acetate (3×100 mL). The combined organic layers were washed with brine (2×100 mL), dried over Na₂SO₄, filtered and concentrated. The residue was purified by flash silica gel chromatography to give tert-butyl 6-bromo-1-oxo-3,4-dihydroisoquinoline-2(1H)-carboxylate (4.2 g, 12.88 mmol, 97% yield) as a white solid. LC-MS (ES⁺, Method A), 0.46 min, m/z 271.9 [M-56]*.

### Step 2: tert-butyl 1-oxo-6-(4,4,5,5-tetramethyl-1,3,2-dioxaborolan-2-yl)-3,4-dihydroisoquinoline-2(1H)-carboxylate

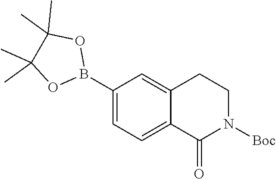 [SMILES: CC(C)(C)OC(=O)N1CCc2cc(B3OC(C)(C)C(C)(C)O3)ccc2C1=O]

To a mixture of tert-butyl 6-bromo-1-oxo-3,4-dihydroisoquinoline-2(1H)-carboxylate (4.2 g, 12.88 mmol) and 4,4,4′,4′,5,5,5′,5′-octamethyl-2,2′-bi(1,3,2-dioxaborolane) (3.92 g, 15.45 mmol) in dioxane (45 mL) was added Pd(dppf)Cl₂ (942.15 mg, 1.29 mmol), AcOK (2.53 g, 25.75 mmol) in one portion at 25° C. under N₂. The mixture was stirred at 90° C. for 16 hr. The reaction mixture was poured into water (100 mL), extracted with Ethyl acetate (3×100 mL). The combined organic layers were washed with brine (2×100 mL), dried over Na₂SO₄, filtered and concentrated. The residue was purified by flash silica gel chromatography to give tert-butyl 1-oxo-6-(4,4,5,5-tetramethyl-1,3,2-dioxaborolan-2-yl)-3,4-dihydroisoquinoline-2(1H)-carboxylate (2.6 g, 6.97 mmol, 54% yield) as a white solid. LC-MS (ES⁺, Method A), 0.50 min, m/z 317.9 [M-56]⁺.

### Step 3: tert-butyl 6-(3-iodo-4-methyl-1H-pyrazol-1-yl)-1-oxo-3,4-dihydroisoquinoline-2(1H)-carboxylate

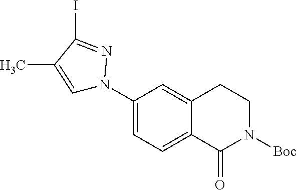 [SMILES: Cc1cn(-c2ccc3c(c2)CCN(C(=O)OC(C)(C)C)C3=O)nc1I]

To a solution of tert-butyl 1-oxo-6-(4,4,5,5-tetramethyl-1,3,2-dioxaborolan-2-yl)-3,4-dihydroisoquinoline-2(1H)-carboxylate (800 mg, 2.14 mmol) and 3-iodo-4-methyl-1H-pyrazole (490.39 mg, 2.36 mmol) in MeCN (10 mL) was added pyridine (339.08 mg, 4.29 mmol) and Cu(OAc)₂ (583.95 mg, 3.22 mmol) and 4A-MS (100 mg, 2.14 mmol) and boric acid (265.06 mg, 4.29 mmol). The mixture was stirred at 60° C. for 16 hr. The reaction mixture was poured into water (50 mL), extracted with ethyl acetate (2×50 mL). The combined organic layer was washed with brine (2×50 mL), dried over anhydrous sodium sulfate, filtered and concentrated under reduced pressure to give a residue. The residue was purified by prep-HPLC (56%-86% MeCN in H₂O) to give tert-butyl 6-(3-iodo-4-methyl-1H-pyrazol-1-yl)-1-oxo-3,4-dihydroisoquinoline-2(1H)-carboxylate (240 mg, 529.48 umol, 25% yield) as a white solid. LC-MS (ES⁺, Method A), 0.51 min, m/z 454.0 [M+H]⁺.

### Step 4: tert-butyl 6-(3-((4-chloro-1-(tetrahydro-2H-pyran-2-yl)-1H-indazol-5-yl)amino)-4-methyl-1H-pyrazol-1-yl)-1-oxo-3,4-dihydroisoquinoline-2(1H)-carboxylate

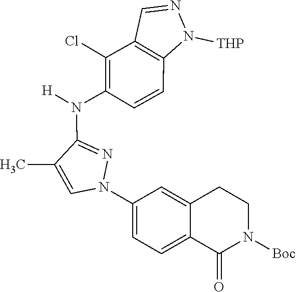

To a mixture of tert-butyl 6-(3-iodo-4-methyl-1H-pyrazol-1-yl)-1-oxo-3,4-dihydroisoquinoline-2(1H)-carboxylate (100 mg, 220.62 umol) and 4-chloro-1-(tetrahydro-2H-pyran-2-yl)-1H-indazol-5-amine (55.53 mg, 220.62 umol) in dioxane (1 mL) was added Xantphos (25.53 mg, 44.12 umol), Cs₂CO₃ (143.76 mg, 441.24 umol) and Pd₂(dba)₃ (20.20 mg, 22.06 umol) in one portion at 25° C. under N2. The mixture was stirred at 100° C. for 16 hr. The reaction mixture was poured into water (20 mL), extracted with ethyl acetate (2×20 mL). The combined organic layer was washed with brine (2×20 mL), dried over anhydrous sodium sulfate, filtered and concentrated under reduced pressure to give a residue. The residue was purified by flash silica gel chromatography to give tert-butyl 6-(3-((4-chloro-1-(tetrahydro-2H-pyran-2-yl)-1H-indazol-5-yl)amino)-4-methyl-1H-pyrazol-1-yl)-1-oxo-3,4-dihydroisoquinoline-2(1H)-carboxylate (100 mg, 136.90 umol, 62% yield) as a yellow solid. LC-MS (ES⁺, Method A), 0.57 min, m/z 577.3 [M+H]⁺.

### Example 83: 6-(3-((4-chloro-1H-indazol-5-yl)amino)-4-methyl-1H-pyrazol-1-yl)-3,4-dihydroisoquinolin-1(2H)-one

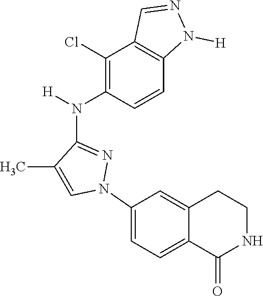 [SMILES: Cc1cn(-c2ccc3c(c2)CCNC3=O)nc1Nc1ccc2[nH]ncc2c1Cl]

To a mixture of tert-butyl 6-(3-((4-chloro-1-(tetrahydro-2H-pyran-2-yl)-1H-indazol-5-yl)amino)-4-methyl-1H-pyrazol-1-yl)-1-oxo-3,4-dihydroisoquinoline-2(1H)-carboxylate (100 mg, 136.90 umol) in DCM (2 mL) was added HCl/dioxane (4 M, 342.24 uL) in one portion. The mixture was stirred at 25° C. for 1 hr. The reaction mixture was concentrated under reduced pressure to remove solvent. The residue was purified by prep-HPLC (31%-61% MeCN in H₂O) to give 6-(3-((4-chloro-1H-indazol-5-yl)amino)-4-methyl-1H-pyrazol-1-yl)-3,4-dihydroisoquinolin-1(2H)-one (26.4 mg, 66.53 umol, 49% yield) as an off-white solid. LC-MS (ES⁺, Method A), 0.40 min, m/z 393.0 [M+H]⁺. ¹H NMR (400 MHz, DMSO-d₆) δ 13.22 (s, 1H) 8.29 (s, 1H) 8.01 (s, 1H) 7.82-7.87 (m, 2H) 7.79 (d, J=8.8 Hz, 1H) 7.59-7.65 (m, 2H) 7.49 (d, J=8.8 Hz, 1H) 7.29 (s, 1H) 3.36-3.40 (m, 2H) 2.93 (t, J=6.4 Hz, 2H) 2.03 (s, 3H).

### General Route or the Synthesis of Example 84

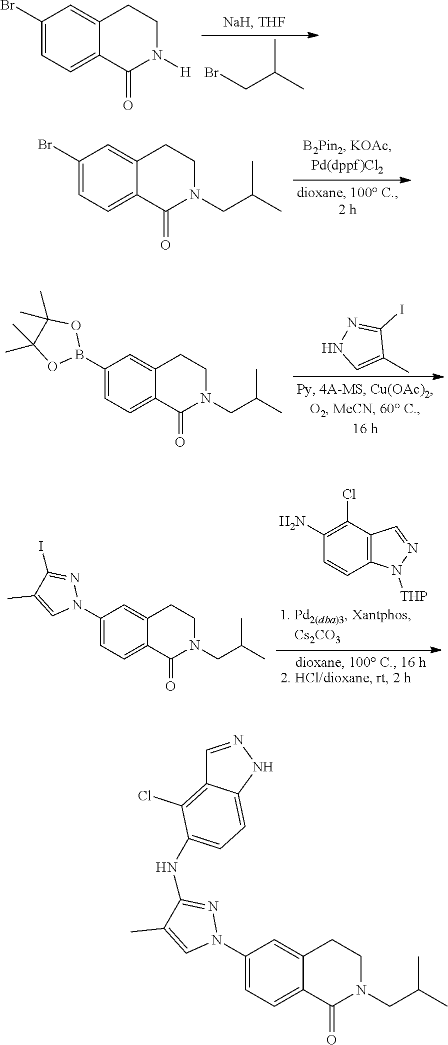

### Step 1: 6-bromo-2-isobutyl-3,4-dihydroisoquinolin-1-one

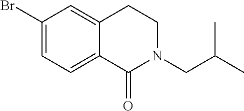 [SMILES: CC(C)CN1CCc2cc(Br)ccc2C1=O]

To a solution of 6-bromo-3,4-dihydro-2H-isoquinolin-1-one (2 g, 8.85 mmol, 1 eq) in THF (20 mL) was added NaH (1.77 g, 44.23 mmol, 60% purity) at 25° C. for 1 hr, and then to the mixture was added 1-bromo-2-methyl-propane (2.42 g, 17.69 mmol, 1.92 mL), KI (5.87 g, 35.39 mmol). The mixture was stirred at 60° C. for 4 hr. The reaction mixture was quenched by saturated NH₄Cl (20 mL) and extracted with EtOAc (3×60 mL). The combined organic layers were washed with brine (30 mL), dried over anhydrous Na₂SO₄, filtered and concentrated under reduced pressure. The residue was purified by flash silica gel chromatography eluting with 0-30% EtOAc in Pet. Ether to give 6-bromo-2-isobutyl-3,4-dihydroisoquinolin-1-one (2 g, 7.09 mmol, 80.12% yield) was obtained as a yellow solid. LC-MS (ES⁺, Method A), 0.467 min, m/z 283.8 [M+H]⁺.

### Step 2: 2-isobutyl-6-(4,4,5,5-tetramethyl-1,3,2-dioxaborolan-2-yl)-3,4-dihydroisoquinolin-1-one

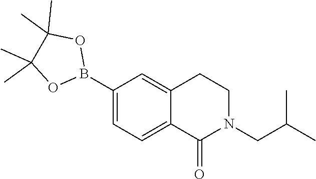 [SMILES: CC(C)CN1CCc2cc(B3OC(C)(C)C(C)(C)O3)ccc2C1=O]

A mixture of 6-bromo-2-isobutyl-3,4-dihydroisoquinolin-1-one (1 g, 3.54 mmol), 4,4,5,5-tetramethyl-2-(4,4,5,5-tetramethyl-1,3,2-dioxaborolan-2-yl)-1,3,2-dioxaborolane (1.35 g, 5.32 mmol), Pd(dppf)Cl₂.CH₂Cl₂ (289.41 mg, 354.39 umol) and KOAc (695.61 mg, 7.09 mmol) in dioxane (10 mL) was degassed and purged with N₂ for 3 times, and then the mixture was stirred at 100° C. for 16 hr under N₂ atmosphere. The reaction mixture was concentrated under reduced pressure to give a residue. The residue was purified by flash silica gel chromatography eluting with 0-30% EtOAc in Pet. Ether to give 2-isobutyl-6-(4,4,5,5-tetramethyl-1,3,2-dioxaborolan-2-yl)-3,4-dihydroisoquinolin-1-one (1 g, 3.04 mmol, 85.71% yield) as a yellow solid. LC-MS (ES⁺, Method A), 0.517 min, m/z 330.0 [M+H]⁺.

### Step 3: 6-(3-iodo-4-methyl-pyrazol-1-yl)-2-isobutyl-3,4-dihydroisoquinolin-1-one

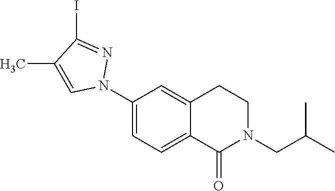 [SMILES: Cc1cn(-c2ccc3c(c2)CCN(CC(C)C)C3=O)nc1I]

To a solution of 2-isobutyl-6-(4,4,5,5-tetramethyl-1,3,2-dioxaborolan-2-yl)-3,4-dihydroisoquinolin-1-one (200 mg, 607.46 umol) and 3-iodo-4-methyl-1H-pyrazole (113.72 mg, 546.71 umol) in MeCN (5 mL) was added Py (72.07 mg, 911.19 umol, 73.55 uL), Cu(OAc)2 (220.67 mg, 1.21 mmol), 4 Å MS (100 mg) and boric acid (75.12 mg, 1.21 mmol). The mixture was stirred at 60° C. for 16 hr. The reaction mixture was concentrated under reduced pressure to give a residue. The residue was purified by preparative HPLC (55-85% MeCN in H₂O) to give 6-(3-iodo-4-methyl-pyrazol-1-yl)-2-isobutyl-3,4-dihydroisoquinolin-1-one (40 mg, 97.74 umol, 16.09% yield) as a colorless solid. LC-MS (ES⁺, Method A), 0.543 min, m/z 410.0 [M+H]⁺.

### Step 4: 6-[3-[(4-chloro-1-tetrahydropyran-2-yl-indazol-5-yl)amino]-4-methyl-pyrazol-1-yl]-2-isobutyl-3,4-dihydroisoquinolin-1-one

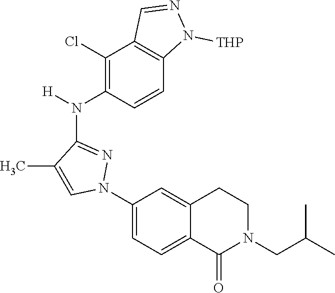

A mixture of 6-(3-iodo-4-methyl-pyrazol-1-yl)-2-isobutyl-3,4-dihydroisoquinolin-1-one (100 mg, 244.34 umol, 1 eq), 4-chloro-1-tetrahydropyran-2-yl-indazol-5-amine (61.50 mg, 244.34 umol), Pd₂(dba)₃ (11.19 mg, 12.22 umol), Xantphos (14.14 mg, 24.43 umol) and Cs₂CO₃ (159.22 mg, 488.68 umol) in dioxane (4 mL) was degassed and purged with N₂ for 3 times, and then the mixture was stirred at 100° C. for 16 hr under N₂ atmosphere. The reaction mixture was diluted with H₂O (5 mL) and extracted with EtOAc (3×15 mL). The combined organic layers were washed with brine (15 mL), dried over Na₂SO₄, filtered and concentrated under reduced pressure to give a residue. The residue was purified by preparative TLC eluting with 50% EtOAc in Pet. Ether to give 6-[3-[(4-chloro-1-tetrahydropyran-2-yl-indazol-5-yl)amino]-4-methyl-pyrazol-1-yl]-2-isobutyl-3,4-dihydroisoquinolin-1-one (40 mg, 75.04 umol, 30.71% yield) as a red solid. LC-MS (ES⁺, Method A), 0.612 min, m/z 533.3 [M+H]⁺.

### Example 84: 6-[3-[(4-chloro-1H-indazol-5-yl)amino]-4-methyl-pyrazol-1-yl]-2-isobutyl-3,4-dihydroisoquinolin-1-one

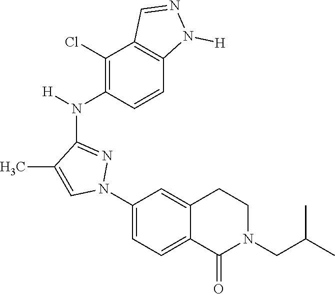

A mixture of 6-[3-[(4-chloro-1-tetrahydropyran-2-yl-indazol-CI 5-yl)amino]-4-methyl-pyrazol-1-yl]-2-isobutyl-3,4-dihydroisoquinolin-1-one (40 mg, 75.04 umol) in HCl/dioxane (4 M, 3 mL) was stirred at 25 H₃C N ° C. for 2 hr. The reaction mixture was concentrated under reduced pressure to give a residue. The residue was purified by preparative HPLC (50-80% MeCN in H₂O) to give 6-[3-[(4-chloro-1H-indazol-5-yl)amino]-4-methyl-pyrazol-1-yl]-2-isobutyl-3,4-dihydroisoquinolin-1-one (8.5 mg, 17.42 umol, 23.21% yield, 92% purity) as a pink solid. LC-MS (ES⁺, Method A), 0.52 min, m/z 449.1 [M+H]⁺. tH 1H NMR (400 MHz, DMSO-d6) δ 13.24 (s, 1H), 8.29 (s, 1H), 8.02 (s, 1H), 7.87 (d, J=8.4 Hz, 1H), 7.78 (d, J=8.8 Hz, 1H), 7.68-7.57 (m, 2H), 7.49 (br d, J=9.2 Hz, 1H), 7.30 (s, 1H), 3.53 (t, J=6.4 Hz, 2H), 3.29 (d, J=7.6 Hz, 2H), 2.98 (t, J=6.0 Hz, 2H), 2.11-1.92 (m, 4H), 0.88 (d, J=6.8 Hz, 6H).

### General Route or the Synthesis of Example 85-89

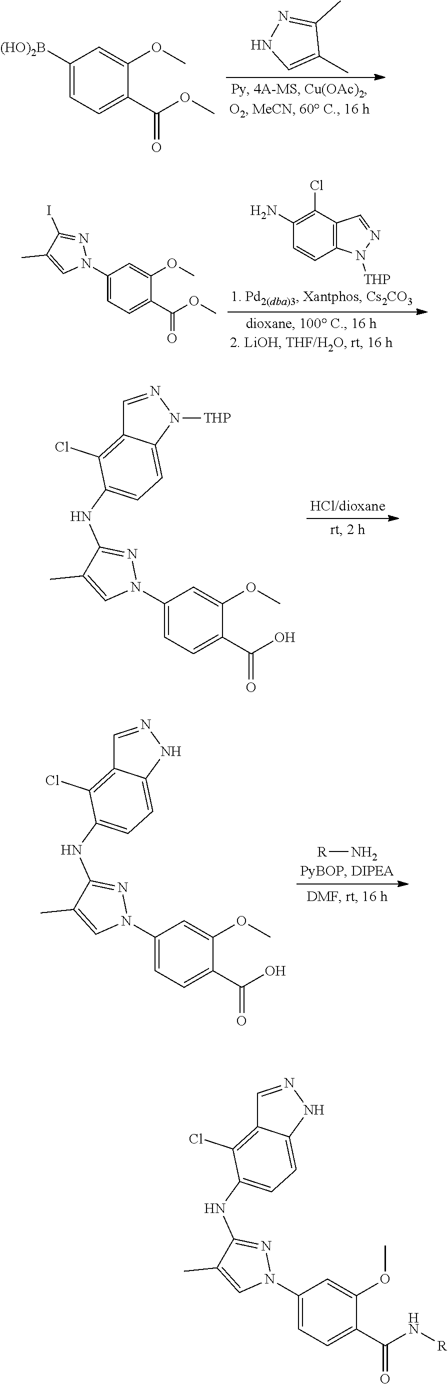

### Step 1: methyl 4-(3-iodo-4-methyl-pyrazol-1-yl)-2-methoxy-benzoate

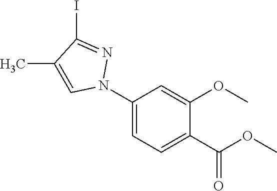

To a solution of (3-methoxy-4-methoxycarbonyl-phenyl)boronic acid (2 g, 9.52 mmol, 1 eq) and 3-iodo-4-methyl-1H-pyrazole (1.98 g, 9.52 mmol, 1 eq) in THE (20 mL) was added Py (1.51 g, 19.05 mmol, 1.54 mL, 2 eq), 4A MS (1 g, 3.66 mmol), Cu(OAc)₂ (2.59 g, 14.29 mmol, 1.5 eq) and BORIC ACID (1.18 g, 19.05 mmol, 2 eq). The mixture was stirred at 60° C. for 16 hr. The reaction mixture was filterd to remove 4A MS and concentrated under reduced pressure to give a residue. The residue was purified by flash silica gel chromatography eluting with 0-25% EtOAc in Pet. Ether to give and prep-HPLC (50%-80% MeCN in H₂O) to give methyl 4-(3-iodo-4-methyl-pyrazol-1-yl)-2-methoxy-benzoate (1.6 g, 4.30 mmol, 45.14% yield) as a white solid. LC-MS (ES⁺, Method A), 0.50 min, m/z 373.0 [M+H]⁺.

### Step 2: methyl 4-[3-[(4-chloro-1-tetrahydropyran-2-yl-indazol-5-yl)amino]-4-methyl-pyrazol-1-yl]-2-methoxy-benzoate

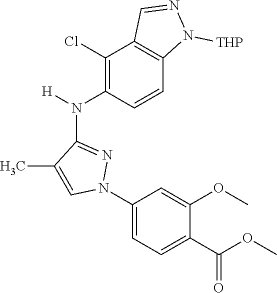

A mixture of methyl 4-(3-iodo-4-methyl-pyrazol-1-yl)-2-methoxy-benzoate (800 mg, 2.15 mmol), 4-chloro-1-tetrahydropyran-2-yl-indazol-5-amine (703.41 mg, 2.79 mmol), Pd₂(dba)₃ (196.85 mg, 214.96 umol), Xantphos (248.76 mg, 429.93 umol) and Cs₂CO₃ (1.40 g, 4.30 mmol) in dioxane (8 mL) was degassed and purged with N₂ for 3 times, and then the mixture was stirred at 100° C. for 16 hr under N₂ atmosphere. The reaction mixture was concentrated under reduced pressure. The residue was purified by flash silica gel chromatography eluting with 25-33% EtOAc in Pet. Ether to give methyl 4-[3-[(4-chloro-1-tetrahydropyran-2-yl-indazol-5-yl)amino]-4-methyl-pyrazol-1-yl]-2-methoxy-benzoate (1.07 g, 2.16 mmol) as a yellow solid. LC-MS (ES⁺, Method A), 0.58 min, m/z 496.1 [M+H]⁺.

### Step 3: 4-[3-[(4-chloro-1-tetrahydropyran-2-yl-indazol-5-yl)amino]-4-methyl-pyrazol-1-yl]-2-methoxy-benzoic acid

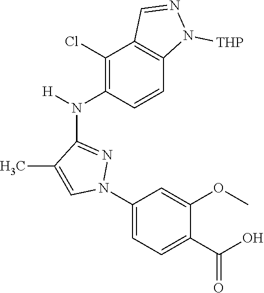

To a solution of methyl 4-[3-[(4-chloro-1-tetrahydropyran-2-yl-indazol-5-yl)amino]-4-methyl-pyrazol-1-yl]-2-methoxy-benzoate (1.07 g, 2.16 mmol) in THE (10 mL) and H₂O (10 mL) was added LiOH·H₂O (271.60 mg, 6.47 mmol). The mixture was stirred at 25° C. for 2 hr. The reaction mixture was diluted with H₂O (20 mL) and extracted with EtOAc (3×20 mL). The combined organic layers were washed with brine (20 mL) and dried over Na₂SO₄, filtered and concentrated under reduced pressure to give 4-[3-[(4-chloro-1-tetrahydropyran-2-yl-indazol-5-yl)amino]-4-methyl-pyrazol-1-yl]-2-methoxy-benzoic acid (800 mg, 1.66 mmol, 76.94% yield) as a blue solid. LC-MS (ES⁺, Method A), 0.53 min, m/z 482.2 [M+H]⁺.

### Step 4: 4-[3-[(4-chloro-1H-indazol-5-yl)amino]-4-methyl-pyrazol-1-yl]-2-methoxy-benzoic acid

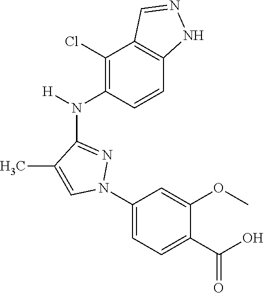

To a solution of 4-[3-[(4-chloro-1-tetrahydropyran-2-yl-indazol-5-yl)amino]-4-methyl-pyrazol-1-yl]-2-methoxy-benzoic acid (800 mg, 1.66 mmol) in HCl/dioxane (4 M, 10 mL). The mixture was stirred at 25° C. for 2 hr. The reaction mixture was concentrated under H reduced pressure to give 4-[3-[(4-chloro-1H-indazol-5-yl)amino]-4-methyl-pyrazol-1-yl]-2-methoxy-benzoic acid (700 mg, 1.61 mmol, 97.10% yield, HCl salt) as a green solid. LC-MS (ES⁺, Method A), 0.45 min, m/z 398.0 [M+H]⁺.

### Example 85: 4-(3-((4-chloro-1H-indazol-5-yl)amino)-4-methyl-1H-pyrazol-1-yl)-N-isopropyl-2-methoxybenzamide

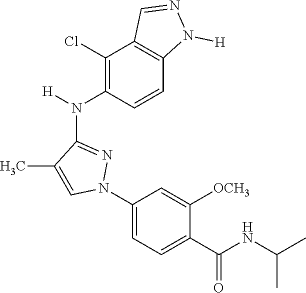

To a solution of 4-(3-((4-chloro-1H-indazol-5-yl)amino)-4-methyl-1H-pyrazol-1-yl)-2-methoxybenzoic acid (80 mg, 201.10 umol), propan-2-amine (13.08 mg, 221.21 umol) in DMF (1 mL) was added pybop (209.30 mg, 402.20 umol) and DIEA (51.98 mg, 402.20 umol). The mixture was stirred at 20° C. for 16 hr. The mixture reaction was purified by prep-HPLC (45%-75% MeCN in H₂O) to give 4-(3-((4-chloro-1H-indazol-5-yl)amino)-4-methyl-1H-pyrazol-1-yl)-N-isopropyl-2-methoxybenzamide (15.1 mg, 33.47 umol, 17% yield) as an off-white solid. LC-MS (ES⁺, Method A), 0.59 min, m/z 439.2 [M+H]⁺. ¹H NMR (400 MHz, DMSO-d₆) δ 13.42-12.98 (m, 1H), 8.36 (s, 1H), 8.01 (s, 1H), 7.86-7.78 (m, 2H), 7.73 (d, J=9.2 Hz, 1H), 7.49 (d, J=9.2 Hz, 1H), 7.40-7.27 (m, 3H), 4.06 (d, J=6.8, 13.7 Hz, 1H), 3.96 (s, 3H), 2.01 (s, 3H), 1.17 (d, J=6.4 Hz, 6H).

Compounds prepared in a similar manner to that set out above are given below in Table 20

TABLE 20

| Example | Structure | Method | LCMS | ¹H NMR |
|---|---|---|---|---|
| 86 | 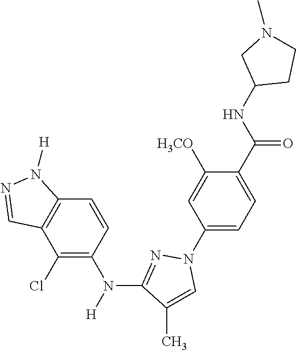 [SMILES: COc1cc(-n2cc(C)c(Nc3ccc4[nH]ncc4c3Cl)n2)ccc1C(=O)NC1CCN(C)C1] | F | LC-MS (ES+, Method A), 0.67 min, m/z 480.3 [M + H]⁺. | ¹H NMR (400 MHz, DMSO-d₆) δ 13.22 (s, 1H), 8.37 (s, 1H), 8.26 (s, 1H), 8.08 (d, J = 7.2 Hz, 1H), 8.01 (s, 1H), 7.81 (d, J = 8.4 Hz, 1H), 7.74 (d, J = 8.8 Hz, 1H), 7.48 (d, J = 8.8 Hz, 1H), 7.40-7.31 (m, 3H), 4.43-4.32 (m, 1H), 3.96 (s, 3H), 2.68-2.61 (m, 2H), 2.43 (dd, J = 4.4, 9.2 Hz, 1H), 2.34 (d, J = 7.6 Hz, 1H), 2.26 (s, 3H), 2.24-2.14 (m, 1H), 2.01 (s, 3H), 1.70- 1.60 (m, 1H). |
| 87 | 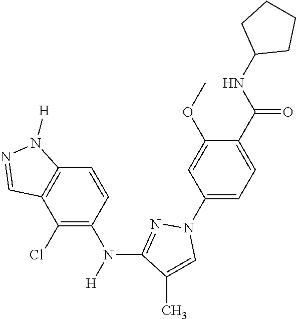 [SMILES: COc1cc(-n2cc(C)c(Nc3ccc4[nH]ncc4c3Cl)n2)ccc1C(=O)NC1CCCC1] | F | LC-MS (ES⁺, Method A), 0.53 min, m/z 465.1 [M + H]⁺ | ¹H NMR (400 MHz, DMSO-d₆) δ 13.22 (s, 1 H), 8.37 (s, 1 H), 8.00 (s, 1 H), 7.93 (d, J = 7.2 Hz, 1 H), 7.81-7.70 (m, 2 H), 7.48 (d, J = 8.8 Hz, 1 H), 7.40-7.31 (m, 3 H), 4.26-4.23 (m, 1 H), 3.95 (s, 3 H), 2.01 (s, 3 H), 1.93-1.84 (m, 2 H), 1.69-1.65 (m, 2 H), 1.61-1.45 (m, 4 H). |
| 88 | 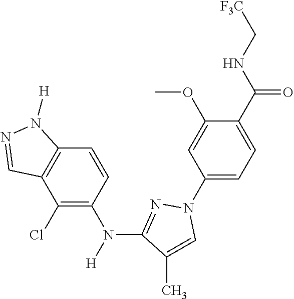 [SMILES: COc1cc(-n2cc(C)c(Nc3ccc4[nH]ncc4c3Cl)n2)ccc1C(=O)NCC(F)(F)F] | F | LC-MS (ES⁺, Method A), 0.51 min, m/z 479.0 [M + H]⁺ | ¹H NMR (400 MHz, DMSO-d₆) δ 13.23 (s, 1 H), 8.57 (t, J = 6.4 Hz, 1 H), 8.40 (s, 1 H), 8.01 (s, 1 H), 7.86 (d, J = 8.8 Hz, 1 H), 7.75 (d, J = 8.8 Hz, 1 H), 7.49 (d, J = 9.2 Hz, 1 H), 7.44- 7.34 (m, 3 H), 4.17-4.06 (m, 2 H), 3.98 (s, H), 2.02 (s, 3 H). |
| 89 | 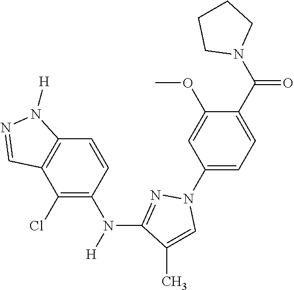 [SMILES: COc1cc(-n2cc(C)c(Nc3ccc4[nH]ncc4c3Cl)n2)ccc1C(=O)N1CCCC1] | F | LC-MS (ES⁺, Method A), 0.48 min, m/z 451.2 [M + H]⁺ | ¹H NMR (400 MHz, DMSO-d₆) δ 13.21 (s, 1 H), 8.34 (s, 1 H), 8.01 (s, 1 H), 7.71 (d, J = 9.2 Hz, 1 H), 7.48 (d, J = 9.2 Hz, 1 H), 7.36 (d, J = 2.0 Hz, 1 H), 7.33-7.27 (m, 2 H), 7.26-7.19 (m, 1 H), 3.87 (s, 3 H), 3.43 (t, J = 6.8 Hz, 2 H), 3.15 (t, J = 6.8 Hz, 2 H), 2.02 (s, 3 H), 1.90-1.73 (m, 4 H). |

General route for the synthesis of Intermediate 1-3

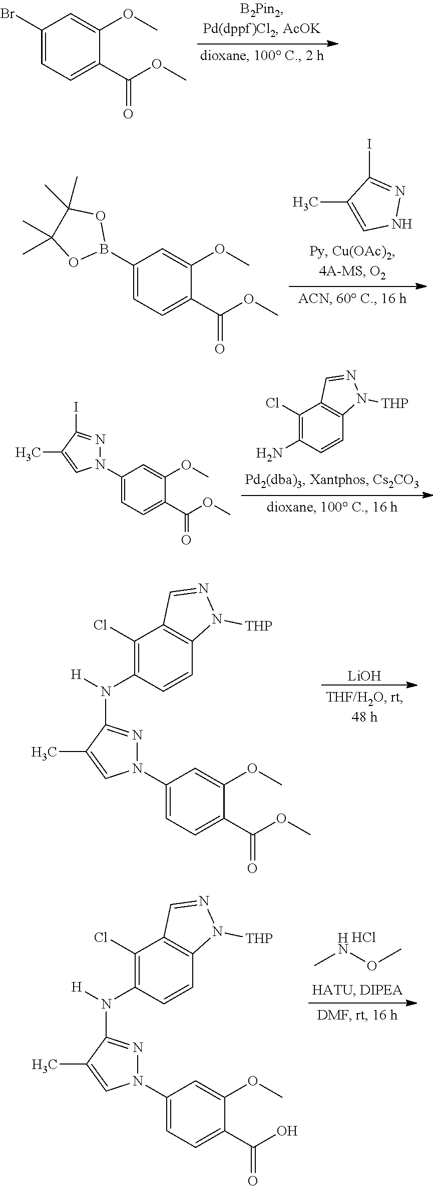

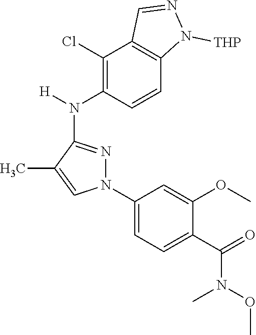

### Step 1: methyl 2-methoxy-4-(4,4,5,5-tetramethyl-1,3,2-dioxaborolan-2-yl)benzoate

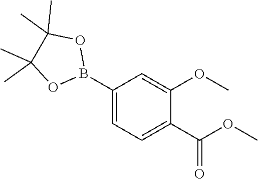

A mixture of methyl 4-bromo-2-methoxy-benzoate (5 g, 20.40 mmol, 1 eq), 4,4,5,5-tetramethyl-2-(4,4,5,5-tetramethyl-1,3,2-dioxaborolan-2-yl)-1,3,2-dioxaborolane (6.22 g, 24.48 mmol, 1.2 eq), Pd(dppf)Cl₂ (746.43 mg, 1.02 mmol, 0.05 eq), KOAc (6.01 g, 61.21 mmol, 3 eq) in dioxane (50 mL) was degassed and purged with N₂ for 3 times, and then the mixture was stirred at 100° C. for 2 hr under N₂ atmosphere. The reaction mixture was concentrated under reduced pressure. The residue was purified by flash silica gel chromatography eluting with 0-9% EtOAc in Pet. Ether to give methyl 2-methoxy-4-(4,4,5,5-tetramethyl-1,3,2-dioxaborolan-2-yl)benzoate (5.9 g, 20.20 mmol, 98.99% yield) as a yellow oil.

### Step 2: methyl 4-(3-iodo-4-methyl-pyrazol-1-yl)-2-methoxy-benzoate

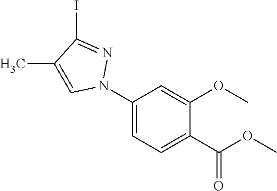

To a solution of methyl 2-methoxy-4-(4,4,5,5-tetramethyl-1,3,2-dioxaborolan-2-yl)benzoate (5.8 g, 19.85 mmol, 1 eq) and 3-iodo-4-methyl-1H-pyrazole (4.13 g, 19.85 mmol, 1 eq) in THF (50 mL) was added Py (3.14 g, 39.71 mmol, 3.20 mL, 2 eq) and 4A MS (2.5 g, 3.66 mmol), Cu(OAc)₂ (5.41 g, 29.78 mmol, 1.5 eq) and boric acid (2.46 g, 39.71 mmol, 2 eq). The mixture was stirred at 60° C. under O₂ atmosphere for 16 hr. The reaction mixture was filter and concentrate under reduced pressure to give a residue. The crude product was purified by reversed phase MPLC (FA conditions) to give methyl 4-(3-iodo-4-methyl-pyrazol-1-yl)-2-methoxy-benzoate (4.5 g, 12.09 mmol, 60.90% yield) was obtained as a yellow solid. LC-MS (ES⁺, Method A), 0.50 min, m/z 373.0 [M+H]⁺.

### Intermediate 1: methyl 4-[3-[(4-chloro-1-tetrahydropyran-2-yl-indazol-5-yl) amino]-4-methyl-pyrazol-1-yl]-2-methoxy-benzoate

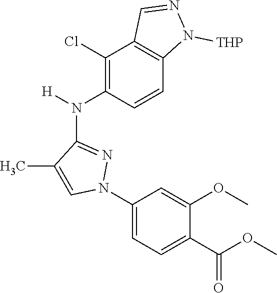

A mixture of methyl 4-(3-iodo-4-methyl-pyrazol-1-yl)-2-methoxy-benzoate (2.3 g, 6.18 mmol, 1 eq), 4-chloro-1-tetrahydropyran-2-yl-indazol-5-amine (2.02 g, 8.03 mmol, 1.3 eq), Pd₂(dba)₃ (565.93 mg, 618.02 umol, 0.1 eq), Xantphos (715.19 mg, 1.24 mmol, 0.2 eq) and Cs₂CO₃ (4.03 g, 12.36 mmol, 2 eq) in dioxane (2 mL) was degassed and purged with N₂ for 3 times, and then the mixture was stirred at 100° C. for 16 hr under N₂ atmosphere. The reaction mixture was filtered and concentrated under reduced pressure to give a residue. The residue was purified by flash silica gel chromatography eluting with 0-60% EtOAc in Pet. Ether to give methyl 4-[3-[(4-chloro-1-tetrahydropyran-2-yl-indazol-5-yl)amino]-4-methyl-pyrazol-1-yl]-2-methoxy-benzoate (2.1 g, 4.23 mmol, 68.51% yield) as a brown solid. LC-MS (ES⁺, Method A), 0.55 min, m/z 496.2 [M+H]⁺.

### Intermediate 2: 4-[3-[(4-chloro-1-tetrahydropyran-2-yl-indazol-5-yl) amino]-4-methyl-pyrazol-1-yl]-2-methoxy-benzoic acid

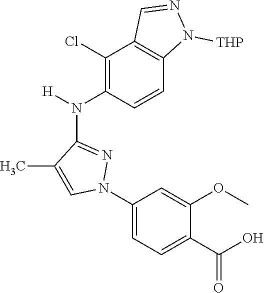

To a solution of methyl 4-[3-[(4-chloro-1-tetrahydropyran-2-yl-indazol-5-yl)amino]-4-methyl-pyrazol-1-yl]-2-methoxy-benzoate (2.1 g, 4.23 mmol, 1 eq) in THF (10 mL) and H₂O (10 nL) was added LiOH·H₂O (888.42 mg, 21.17 mmol, 5 eq). The mixture was stirred at 25° C. for 16 hr. The reaction mixture was diluted with HCl (1 N, 20 mL) and extracted with EtOAc (30 mL*3). The combined organic layers were dried over Na₂SO₄, filtered and concentrated under reduced pressure to give 4-[3-[(4-chloro-1-tetrahydropyran-2-yl-indazol-5-yl)amino]-4-methyl-pyrazol-1-yl]-2-methoxy-benzoic acid (2 g, 4.15 mmol, 98.01% yield) as a brown solid. LC-MS (ES⁺, Method A), 0.59 min, m/z 482.2 [M+H]⁺.

### Intermediate 3: 4-(3-((4-chloro-1-(tetrahydro-2H-pyran-2-yl)-1H-indazol-5-yl)amino)-4-methyl-1H-pyrazol-1-yl)-N, 2-dimethoxy-N-methylbenzamide

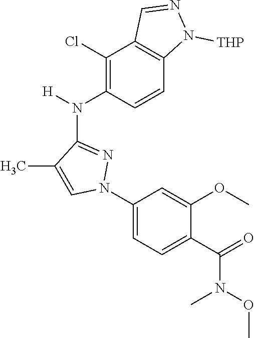

To a solution of 4-[3-[(4-chloro-1-tetrahydropyran-2-yl-indazol-5-yl)amino]-4-methyl-pyrazol-1-yl]-2-methoxy-benzoic acid (1.3 g, 2.70 mmol, 1 eq) and N-methoxymethanamine (526.25 mg, 5.39 mmol, 2 eq, HCl) in DMF (10 mL) was added HATU (1.54 g, 4.05 mmol, 1.5 eq) and DIEA (1.74 g, 13.49 mmol, 2.35 mL, 5 eq). The mixture was stirred at 25° C. for 2 hr. The reaction mixture was diluted with H₂O (30 mL) and extracted with EtOAc (30 mL*3). The combined organic layers were dried over Na₂SO₄, filtered and concentrated under reduced pressure to give a residue. The residue was purified by flash silica gel chromatography eluting with 0-70% EtOAc in Pet. Ether to give 4-[3-[(4-chloro-1-tetrahydropyran-2-yl-indazol-5-yl)amino]-4-methyl-pyrazol-1-yl]-N, 2-dimethoxy-N-methyl-benzamide (1.3 g, 2.48 mmol, 91.80% yield) as a brown oil. LC-MS (ES⁺, Method A), 0.54 min, m/z 525.3 [M+H]⁺*.

General route for the synthesis of Intermediate 4

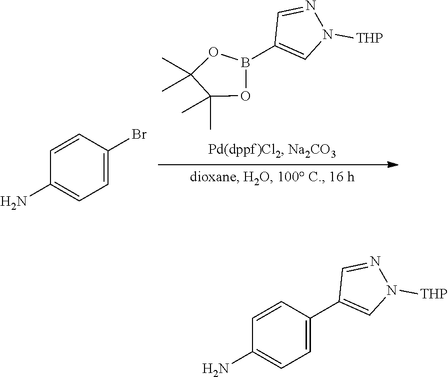

### Intermediate 4: 4-(1-(tetrahydro-2H-pyran-2-yl)-1H-pyrazol-4-yl)aniline

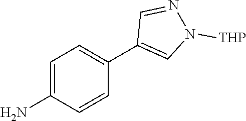

To a solution of 4-bromoaniline (5 g, 29.07 mmol, 1 eq) and 1-tetrahydropyran-2-yl-4-(4,4,5,5-tetramethyl-1,3,2-dioxaborolan-2-yl)pyrazole (8.89 g, 31.97 mmol, 1.1 eq) in dioxane (50 mL) and H₂O (10 mL) was added Pd(dppf)Cl₂.CH₂Cl2 (1.19 g, 1.45 mmol, 0.05 eq) and Na₂CO₃ (6.16 g, 58.13 mmol, 2 eq). The mixture was stirred at 80° C. for 16 hr. The mixture was filtered, and concentrated under reduced pressure affording the residue. The residue was purified by column chromatography eluting with 0-50% EtOAc in Pet. Ether to give 4-(1-tetrahydropyran-2-ylpyrazol-4-yl)aniline (3.3 g, 13.56 mmol, 46.66% yield) as a yellow solid. LC-MS (ES⁺, Method A), 0.250 min, m/z 244.2 [M+H]⁺.

General route for the synthesis of Intermediate 5

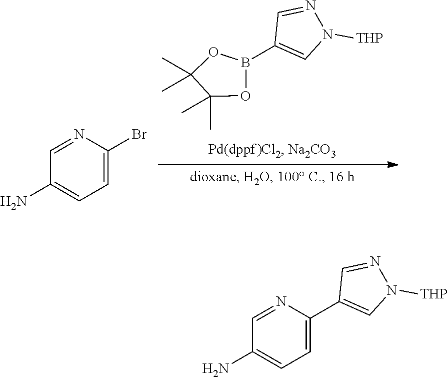

### Intermediate 5: 6-(1-(tetrahydro-2H-pyran-2-yl)-1H-pyrazol-4-yl)pyridin-3-amine

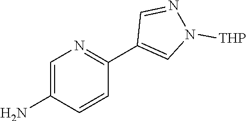

To a solution of 6-chloropyridin-3-amine (5 g, 38.89 mmol, 1 eq) and 1-tetrahydropyran-2-yl-4-(4,4,5,5-tetramethyl-1,3,2-dioxaborolan-2-yl)pyrazole (10.82 g, 38.89 mmol, 1 eq) in dioxane (50 mL) and H₂O (10 mL) was added Na₂CO₃ (8.24 g, 77.79 mmol, 2 eq) and Pd(dppf)Cl₂·CH₂Cl₂ (1.59 g, 1.94 mmol, 0.05 eq). The mixture was stirred at 80° C. for 16 hr. The reaction mixture was filtered and concentrated under reduced pressure to give a residue. The residue was purified by column chromatography eluting with 0-100% EtOAc in Pet. Ether to give 6-(1-tetrahydropyran-2-ylpyrazol-4-yl)pyridin-3-amine (6.8 g, 27.84 mmol, 71.57% yield) was obtained as a yellow solid. LC-MS (ES⁺, Method A), 0.237 min, m/z 245.2 [M+H]⁺.

General route for the synthesis of Intermediate 6

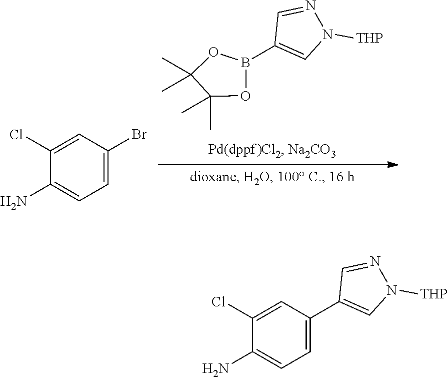

### Intermediate 6: 2-chloro-4-(1-tetrahydropyran-2-ylpyrazol-4-yl)aniline

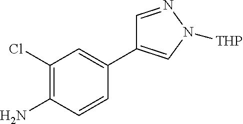

To a solution of 4-bromo-2-chloro-aniline (5 g, 24.22 mmol, 1 eq) and 1-tetrahydropyran-2-yl-4-(4,4,5,5-tetramethyl-1,3,2-dioxaborolan-2-yl)pyrazole (8.08 g, 29.06 mmol, 1.2 eq) in dioxane (50 mL) and H₂O (5 mL) was added Pd(dppf)Cl₂.CH₂Cl₂ (1.98 g, 2.42 mmol, 0.1 eq) and Na₂CO₃ (5.13 g, 48.43 mmol, 2 eq). The mixture was stirred at 100° C. for 16 hr. The reaction mixture was filtered and concentrated under reduced pressure to give a residue. The residue was purified by column chromatography eluting with 0-25% EtOAc in Pet. Ether to give 2-chloro-4-(1-tetrahydropyran-2-ylpyrazol-4-yl)aniline (4.5 g, 16.20 mmol, 66.90% yield) as a yellow solid. LC-MS (ES⁺, Method A), 0.452 min, m/z 278.1 [M+H]⁺.

General route for the synthesis of Intermediates 7 and 8

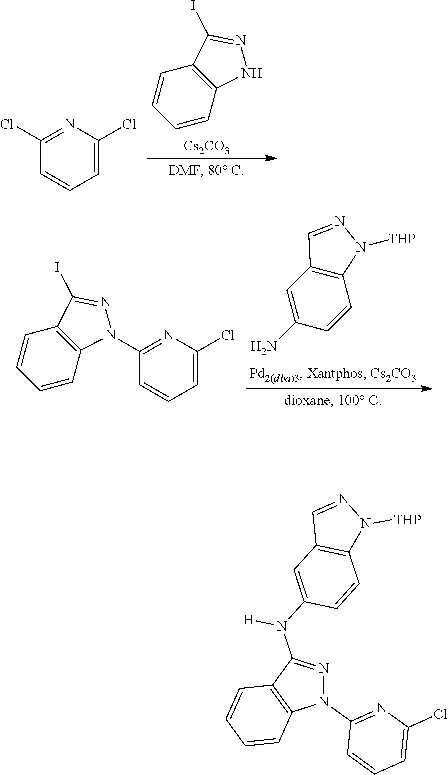

### Intermediate 7: 1-(6-chloropyridin-2-yl)-3-iodo-1H-indazole

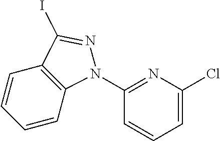 [SMILES: Clc1cccc(-n2nc(I)c3ccccc32)n1]

A mixture of 3-iodo-1H-indazole (0.2 g, 819.56 umo), 2,6-dichloropyridine (130 mg, 819.56 umol,) and Cs₂CO₃ (534 mg, 1.64 mmol) in DMF (5 mL) was degassed and purged with N₂ for 3 times, and then the mixture was stirred at 80° C. for 16 h under N₂ atmosphere. The reaction mixture was partitioned between EtOAc (50 mL) and water (20 mL). The organic phase was separated, washed with brine (20 mL×3), dried over Na₂SO₄, filtered and concentrated under reduced pressure to give a residue. The residue was purified by column chromatography eluting with 20-50% EtOAc in Pet. Ether to give 1-(6-chloropyridin-2-yl)-3-iodo-1H-indazole (0.25 g, 611.71 umol, 75% yield) as a white solid. LC-MS (ES⁺, Method A), 0.87 min, m/z 355.8 [M+H]⁺.

### Intermediate 8: 1-(6-chloropyridin-2-yl)-N-(1-(tetrahydro-2H-pyran-2-yl)-1H-indazol-5-yl)-1H-indazol-3-amine

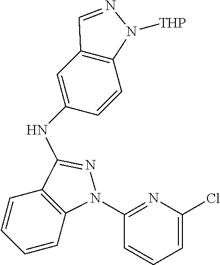

To a solution of 1-(6-chloropyridin-2-yl)-3-iodo-1H-indazole (240 mg, 674.99 μmol) and 1-(tetrahydro-2H-pyran-2-yl)-1H-indazol-5-amine (147 mg, 674.99 μmol) in dioxane (6 mL) was added Xantphos (78 mg, 135.00 μmol) and Pd₂(dba)₃ (62 mg, 67.50 μmol) and Cs₂CO₃ (440 mg, 1.35 mmol) at r.t. The reaction was evacuated, flushed with nitrogen and stirred at 100° C. for 2 hr. The reaction was cooled to r.t. and solvent was removed in vacuo. The residue was partitioned between H₂O (10 mL) and EtOAc (10 mL). The organic layer was separated and the aqueous was extracted with EtOAc (3×10 mL). The combined organics were washed with brine (2×10 mL), dried over sodium sulfate, filtered and the solvent removed in vacuo. The residue was loaded onto silica and purified by column chromatography eluting with 0-33% EtOAc in Pet. Ether to give 1-(6-chloropyridin-2-yl)-N-(1-(tetrahydro-2H-pyran-2-yl)-1H-indazol-5-yl)-1H-indazol-3-amine (220 mg, 494.48 μmol, 73% yield) as a yellow solid. LC-MS (ES⁺, Method A), 0.80 min, m/z 445.3 [M+H]⁺.

General method for the synthesis of Intermediate 9

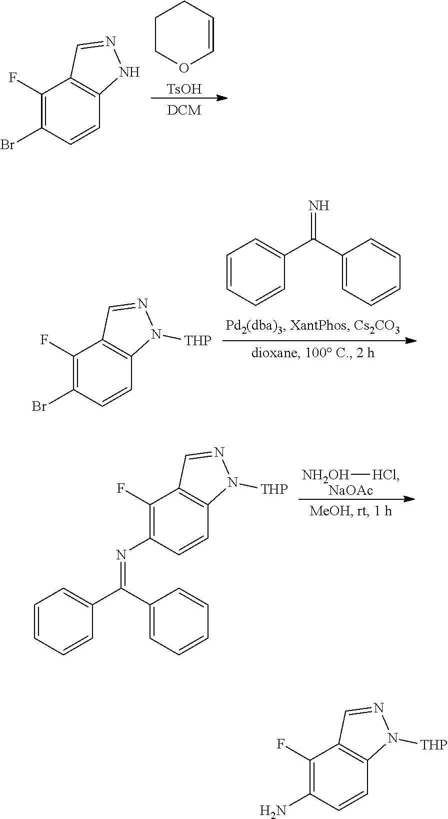

### Step 1: 5-bromo-4-fluoro-1-tetrahydropyran-2-yl-indazole

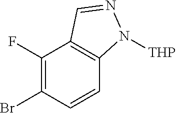

To a solution of 5-bromo-4-fluoro-1H-indazole (2 g, 9.30 mmol, 1 eq) and 3,4-dihydro-2H-pyran (2.35 g, 27.90 mmol, 2.55 mL, 3 eq) in DCM (10 mL) was added TsOH·H₂O (176.93 mg, 930.14 umol, 0.1 eq). The mixture was stirred at 25° C. for 16 hr. The mixture was concentrated under reduced pressure. The residue was purified by flash silica gel chromatography eluting with 0-20% EtOAc in Pet. Ether to afford 5-bromo-4-fluoro-1-tetrahydropyran-2-yl-indazole (2.6 g, 8.69 mmol, 93.44% yield) as a white solid. LC-MS (ES⁺, Method A), 0.459 min, m/z 299.1 [M+H]⁺.

### Step 2: N-(4-fluoro-1-tetrahydropyran-2-yl-indazol-5-yl)-1,1-diphenyl-methanimine

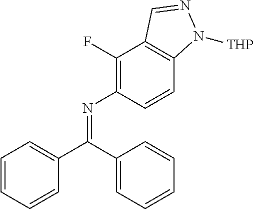

A mixture of 5-bromo-4-fluoro-1-tetrahydropyran-2-yl-indazole (700 mg, 2.34 mmol, 1 eq), diphenylmethanimine (636.14 mg, 3.51 mmol, 589.02 uL, 1.5 eq), Pd₂(dba)₃ (107.14 mg, 117.00 umol, 0.05 eq), Xantphos (135.40 mg, 234.01 umol, 0.1 eq) and Cs₂CO₃ (762.43 mg, 2.34 mmol, 1 eq) in dioxane (10 mL) was degassed and purged with N₂ for 3 times, and then the mixture was stirred at 100° C. for 1 hr under N₂ atmosphere. The mixture was concentrated under reduced pressure. The residue was purified by flash silica gel chromatography eluting with 0-100% EtOAc in Pet. Ether to afford N-(4-fluoro-1-tetrahydropyran-2-yl-indazol-5-yl)-1,1-diphenyl-methanimine (900 mg, 2.25 mmol, 96.28% yield) as a white solid. LC-MS (ES⁺, Method A), 0.555 min, m/z 400.1 [M+H]⁺.

### Intermediate 9: 4-fluoro-1-tetrahydropyran-2-yl-indazol-5-amine: To a solution of N-(4-fluoro-1-tetrahydropyran-2-yl-indazol-5-yl)-1,1-diphenyl-methanimine (900 mg, 2.25 mmol, 1 eq) in MeOH (1 mL) was added hydroxylamine; hydrochloride (1.57 mg, 22.53 umol, 0.01 eq) and sodium; acetate (2.22 mg, 27.04 umol, 0.012 eq). The mixture was stirred at 20° C. for 30 min. The mixture was concentrated under reduced pressure. The residue was purified by flash silica gel chromatography eluting with 0-100% EtOAc in Pet. Ether to afford 4-fluoro-1-tetrahydropyran-2-yl-indazol-5-amine (520 mg, 2.21 mmol, 98.10% yield) as a white solid. LC-MS (ES⁺, Method A), 0.257 min, m/z 236.1 [M+H]⁺

General method for the synthesis of Intermediate 10

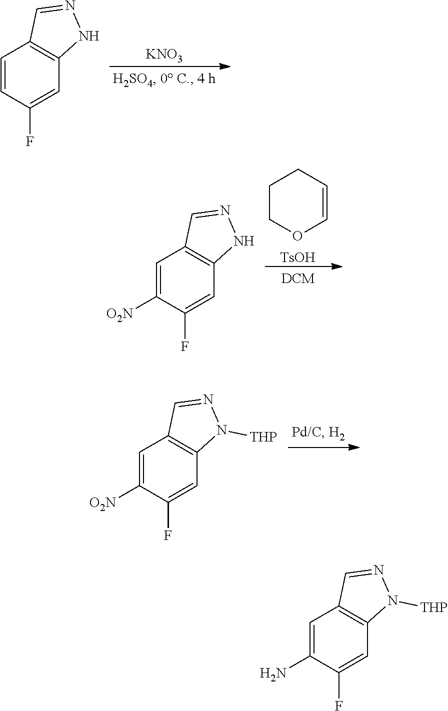

### Step 1: 6-fluoro-5-nitro-1H-indazole

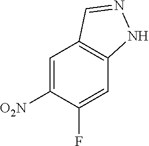 [SMILES: O=[N+]([O-])c1cc2cn[nH]c2cc1F]

To a solution of 6-fluoro-1H-indazole (4 g, 29.38 mmol, 1 eq) in H₂SO₄ (44 mL) was added KNO₃ (3.56 g, 35.21 mmol, 1.20 eq) at 0° C. The mixture was stirred at 0° C. for 0.5 hr. The mixture was cooled to 0° C., basified with saturated NaHCO₃ solution, extracted with EtOAc (50 mL*3), washed with brine and the organic layer was dried over anhydrous Na₂SO₄. The mixture was filtered, and then was concentrated under reduced pressure to give a residue. The residue was purified by flash silica gel chromatography eluting with 0-50% EtOAc in Pet. Ether to afford 6-fluoro-5-nitro-1H-indazole (2.2 g, 11.04 mmol, 37.58% yield) as a white solid. LC-MS (ES⁺, Method A), 0.299 min, m/z 182.1 [M+H]⁺.

### Step 2: 6-fluoro-5-nitro-1-tetrahydropyran-2-yl-indazole

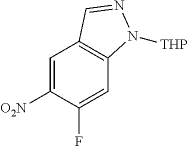

To a solution of 6-fluoro-5-nitro-1H-indazole (1 g, 5.52 mmol, 1 eq) and 3,4-dihydro-2H-pyran (1.39 g, 16.56 mmol, 1.51 mL, 3 eq) in DCM (20 mL) was added TsOH·H₂O (105.02 mg, 552.11 umol, 0.1 eq). The mixture was stirred at 25° C. for 2 hr. The mixture was concentrated under reduced pressure. The residue was purified by flash silica gel chromatography eluting with 0-50% EtOAc in Pet. Ether to afford 6-fluoro-5-nitro-1-tetrahydropyran-2-yl-indazole (1.1 g, 4.15 mmol, 75.12% yield) as a white solid.

### Intermediate 10: 6-fluoro-1-tetrahydropyran-2-yl-indazol-5-amine

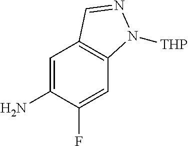

A mixture of 6-fluoro-5-nitro-1-tetrahydropyran-2-yl-indazole (1 g, 3.77 mmol, 1 eq), Pd/C (0.1 g, 10% purity), H₂ (1.00 eq) and MeOH (10 mL) was degassed and purged with N₂ for 3 times, and then the mixture was stirred at 25° C. for 12 hr under H₂ atmosphere. The reaction mixture was filtered and concentrated under reduced pressure to give 6-fluoro-1-tetrahydropyran-2-yl-indazol-5-amine (880 mg, 3.74 mmol, 99.22% yield) as a white solid. LC-MS (ES⁺, Method A), 0.254 min, m/z 236.2 [M+H]⁺.

General method for the synthesis of Intermediate 11

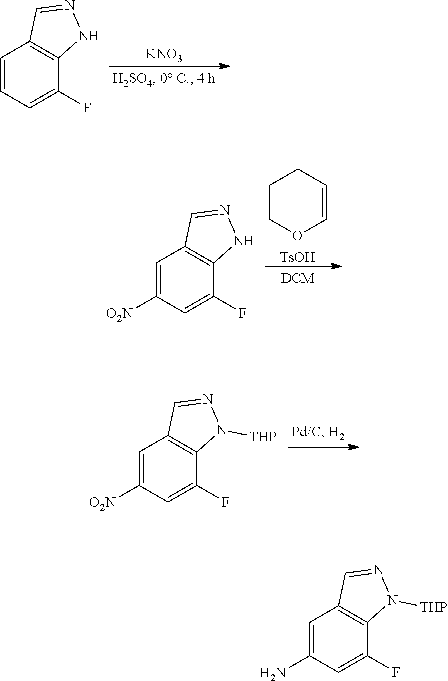

### Step 1: 7-fluoro-5-nitro-1H-indazole

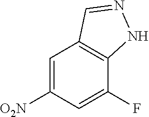 [SMILES: O=[N+]([O-])c1cc(F)c2[nH]ncc2c1]

To a solution of 7-fluoro-1H-indazole (2 g, 14.69 mmol, 1 NH, eq) in H₂SO₄ (44 mL) was added KNO₃ (1.54 g, 15.23 mmol, 1.04 eq) at 0° C. The mixture was stirred at 0° C. for 1 hr. The mixture was cooled to 0° C., basified with saturated NaHCO₃ solution, extracted with EtOAc (50 mL*3), washed with brine and the organic layer was dried over anhydrous Na₂SO₄. The mixture was filtered, and then was concentrated under reduced pressure to give a residue. The residue was purified by flash silica gel chromatography eluting with 0-50% EtOAc in Pet. Ether to afford 7-fluoro-5-nitro-1H-indazole (2.5 g, 13.80 mmol, 93.95% yield) as a yellow solid. LC-MS (ES⁺, Method A), 0.302 min, m/z 182.1 [M+H]⁺.

### Step 2: 7-fluoro-5-nitro-1-tetrahydropyran-2-yl-indazole

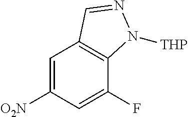

To a solution of 7-fluoro-5-nitro-1H-indazole (2.2 g, 12.15 mmol, 1 eq) and 3,4-dihydro-2H-pyran (3.07 g, 36.44 mmol, 3.33 mL, 3 eq) in DCM (5 mL) was added TsOH·H₂O (231.04 mg, 1.21 mmol, 0.1 eq). The mixture was stirred at 25° C. for 16 hr. The mixture was concentrated under reduced pressure. The residue was purified by flash silica gel chromatography eluting with 0-50% EtOAc in Pet. Ether to afford 7-fluoro-5-nitro-1-tetrahydropyran-2-yl-indazole (2.8 g, 10.56 mmol, 86.91% yield) as a white solid. LC-MS (ES⁺, Method A), 0.312 min, m/z 266.1 [M+H]⁺.

### Intermediate 11: 7-fluoro-1-tetrahydropyran-2-yl-indazol-5-amine

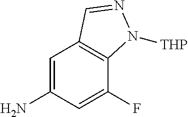

A mixture of 7-fluoro-5-nitro-1-tetrahydropyran-2-yl-indazole (100 mg, 377.02 umol, 1 eq), Pd/C (0.01 g, 10% purity), H₂ (7.54 mmol) in MeOH (20 mL) was degassed and purged with N₂ for 3 times, and then the mixture was stirred at 25° C. for 16 hr under H₂ atmosphere. The reaction mixture was filtered and concentrated under reduced pressure to give 7-fluoro-1-tetrahydropyran-2-yl-indazol-5-amine (80 mg, 340.05 umol, 90.20% yield) as a white solid. LC-MS (ES⁺, Method A), 0.337 min, m/z 236.1 [M+H]⁺.

General method for the synthesis of Intermediate 12

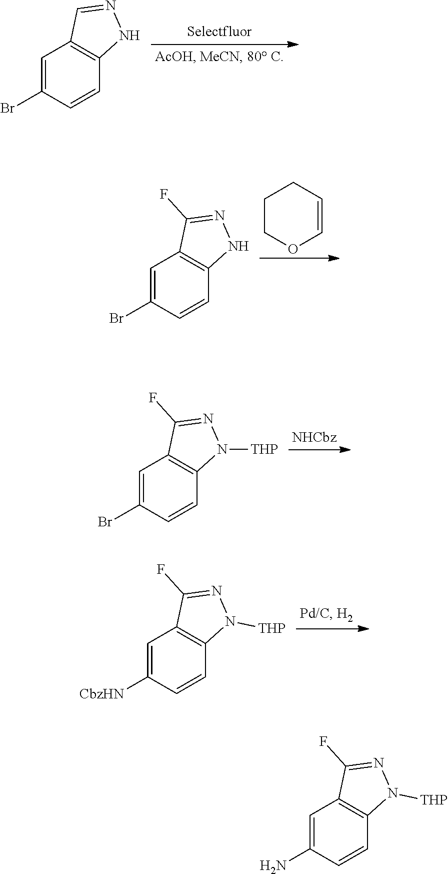

### Step 1: 5-bromo-3-fluoro-1H-indazole

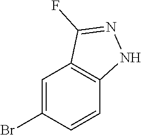 [SMILES: Fc1n[nH]c2ccc(Br)cc12]

To a solution of 5-bromo-1H-indazole (2 g, 10.15 mmol, 1 eq) and 1-(chloromethyl)-4-fluoro-1,4-diazoniabicyclo[2.2.2]octane; ditetrafluoroborate (7.19 g, 20.30 mmol, 2 eq) in AcOH (30 mL) was added MeCN (300 mL). The mixture was stirred at 80° C. for 12 hr. The mixture was concentrated under reduced pressure. The residue was purified by flash silica gel chromatography eluting with 0-50% EtOAc in Pet. Ether to afford 5-bromo-3-fluoro-1H-indazole (2 g, 9.30 mmol, 91.63% yield) as a white solid. LC-MS (ES⁺, Method A), 0.410 min, m/z 214.9 [M+H]⁺.

### Step 2: 5-bromo-3-fluoro-1-tetrahydropyran-2-yl-indazole

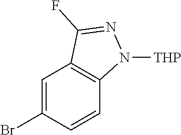

To a solution of 5-bromo-3-fluoro-1H-indazole (1.2 g, 5.58 mmol, 1 eq) and 3,4-dihydro-2H-pyran (1.41 g, 16.74 mmol, 1.53 mL, 3 eq) in DCM (10 mL) was added TsOH·H₂O (106.16 mg, 558.08 umol, 0.1 eq). The mixture was stirred at 25° C. for 1 hr. The mixture was concentrated under reduced pressure. The residue was purified by flash silica gel chromatography eluting with 0-50% EtOAc in Pet. Ether to afford 5-bromo-3-fluoro-1-tetrahydropyran-2-yl-indazole (1.3 g, 4.35 mmol, 77.87% yield) as a white solid.

### Step 3: benzyl N-(3-fluoro-1-tetrahydropyran-2-yl-indazol-5-yl)carbamate

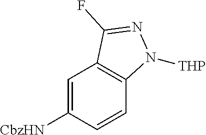

A mixture of 5-bromo-3-fluoro-1-tetrahydropyran-2-yl-indazole (500 mg, 1.67 mmol, 1 eq), benzyl carbamate (757.99 mg, 5.01 mmol, 3 eq), Pd₂(dba)₃ (76.53 mg, 83.57 umol, 0.05 eq), Xantphos (96.71 mg, 167.15 umol, 0.1 eq) and Cs₂CO₃ (544.60 mg, 1.67 mmol, 1 eq) in dioxane (10 mL) was degassed and purged with N₂ for 3 times, and then the mixture was stirred at 100° C. for 2 hr under N₂ atmosphere. The reaction mixture was filtered and concentrated under reduced pressure to give a residue. The residue was purified by flash silica gel chromatography eluting with 0-100% EtOAc in Pet. Ether to afford benzyl N-(3-fluoro-1-tetrahydropyran-2-yl-indazol-5-yl)carbamate (350 mg, 947.51 umol, 56.69% yield) as a white solid. LC-MS (ES⁺, Method A), 0.504 min, m/z 369.2 [M+H]⁺.

### Intermediate 12: 3-fluoro-1-tetrahydropyran-2-yl-indazol-5-amine

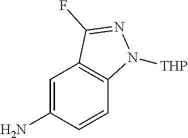

A mixture of benzyl N-(3-fluoro-1-tetrahydropyran-2-yl-indazol-TH 5-yl)carbamate (300 mg, 812.15 umol, 1 eq), Pd/C (0.03 g, 10% purity) in MeOH (10 mL) was degassed and purged with N₂ for 3 times, and then H₂N the mixture was stirred at 25° C. for 12 hr under H₂ atmosphere. The reaction mixture was filtered and concentrated under reduced pressure to give 3-fluoro-1-tetrahydropyran-2-yl-indazol-5-amine (150 mg, 637.60 umol, 78.51% yield) was obtained as a white solid. LC-MS (ES⁺, Method A), 0.271 min, m/z 236.1 [M+H]⁺.

General method for the synthesis of Examples 90-116

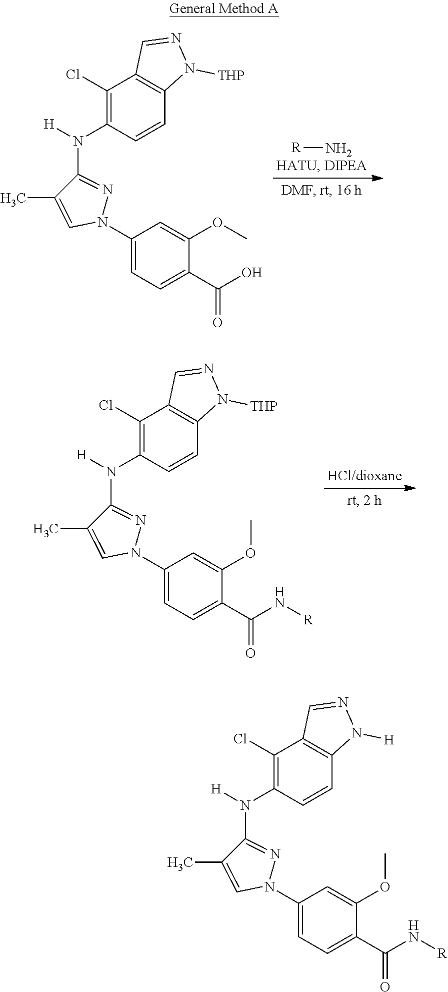

### Step 1: 4-[3-[(4-chloro-1-tetrahydropyran-2-yl-indazol-5-yl) amino]-4-methyl-pyrazol-1-yl]-2-methoxy-N-(1-methylpyrazol-4-yl)benzamide

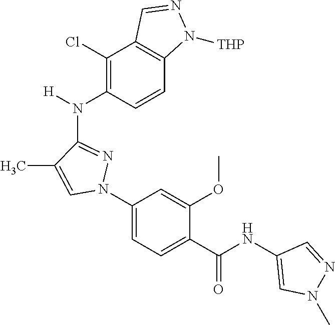

To a solution of 4-[3-[(4-chloro-1-tetrahydropyran-2-yl-indazol-5-yl)amino]-4-methyl-pyrazol-1-yl]-2-methoxy-benzoic acid (1.2 g, 2.49 mmol, 1 eq) and 1-methylpyrazol-4-amine (290.19 mg, 2.99 mmol, 1.2 eq) in DMF (8 mL) was added DIPEA (1.61 g, 12.45 mmol, 2.17 mL, 5 eq) and HATU (1.14 g, 2.99 mmol, 1.2 eq). The mixture was stirred at 25° C. for 2 hr. The reaction mixture filtered and concentrated under reduced pressure to give a residue. The residue was purified by preparative HPLC (56-86% MeCN in H₂O) to give 4-[3-[(4-chloro-1-tetrahydropyran-2-yl-indazol-5-yl)amino]-4-methyl-pyrazol-1-yl]-2-methoxy-N-(1-methylpyrazol-4-yl)benzamide (1.2 g, 2.14 mmol, 85.90% yield) as a brown solid. LC-MS (ES*, Method A), 0.52 min, m/z 561.4 [M+H]⁺.

### Example 90: 4-[3-[(4-chloro-1H-indazol-5-yl) amino]-4-methyl-pyrazol-1-yl]-2-methoxy-N-(1-methylpyrazol-4-yl)benzamide

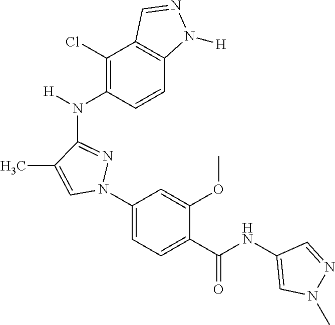

To a solution of 4-[3-[(4-chloro-1-tetrahydropyran-2-yl-indazol-5-yl)amino]-4-methyl-pyrazol-1-yl]-2-methoxy-N-(1-methylpyrazol-4-yl)benzamide (1.2 g, 2.14 mmol, 1 eq) in HCl/dioxane (15 mL) was stirred at 25° C. for 2 hr. The reaction mixture was concentrated under reduced pressure to give a residue. The crude product was triturated with MeOH at 25° C. for 30 min to give 4-[3-[(4-chloro-1H-indazol-5-yl)amino]-4-methyl-pyrazol-1-yl]-2-methoxy-N-(1-methylpyrazol-4-yl)benzamide (796.3 mg, 1.52 mmol, 71.03% yield, 98% purity, HCl) was obtained as a green solid. LC-MS (ES′, Method A), 0.47 min, m/z 477.3 [M+H]⁺.¹H NMR (400 MHz, DMSO-d₆) δ=13.22 (br s, 1H), 9.92 (s, 1H), 8.39 (s, 1H), 8.02 (s, 2H), 7.83-7.72 (m, 2H), 7.57 (s, 1H), 7.49 (d, J=8.8 Hz, 1H), 7.45-7.31 (m, 3H), 3.99 (s, 3H), 3.81 (s, 3H), 2.02 (s, 3H).

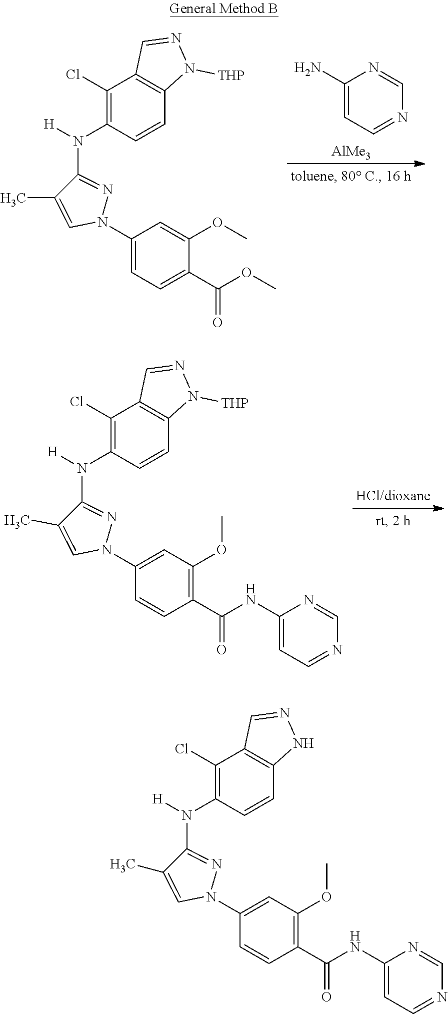

### Step 1: 4-(3-((4-chloro-1-(tetrahydro-2H-pyran-2-yl)-1H-indazol-5-yl)amino)-4-methyl-1H-pyrazol-1-yl)-2-methoxy-N-(pyrimidin-4-yl)benzamide

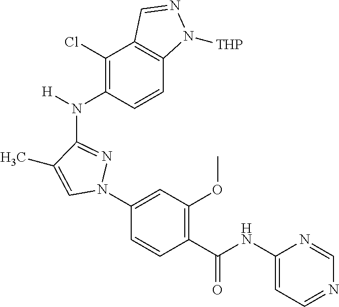

To a solution of pyrimidin-4-amine (95.88 mg, 1.01 mmol, 5 eq) in toluene (1 mL) was added dropwise AlMe₃ (2 M, 504.08 uL, 5 eq) at 0° C. After addition, the mixture was stirred at 25° C. for 2 hr, and then methyl 4-[3-[(4-chloro-1-tetrahydropyran-2-yl-indazol-5-yl)amino]-4-methyl-pyrazol-1-yl]-2-methoxy-benzoate (100 mg, 201.63 umol, 1 eq) in toluene (1 mL) was added dropwise at 25° C. The resulting mixture was stirred at 80° C. for 14 hr. The reaction mixture was quenched with water (2 mL) and extracted with EtOAc (2 mL*3). The extracts are combined, dried over Na₂SO₄ and concentrated in vacuo to give 4-[3-[(4-chloro-1-tetrahydropyran-2-yl-indazol-5-yl)amino]-4-methyl-pyrazol-1-yl]-2-methoxy-N-pyrimidin-4-yl-benzamide (100 mg, 178.89 umol, 88.72% yield) as a yellow solid. LC-MS (ES⁺, Method A), 0.54 min, m/z 559.5 [M+H]⁺*.

### Example 91: 4-(3-((4-chloro-1H-indazol-5-yl)amino)-4-methyl-1H-pyrazol-1-yl)-2-methoxy-N-(pyrimidin-4-yl)benzamide

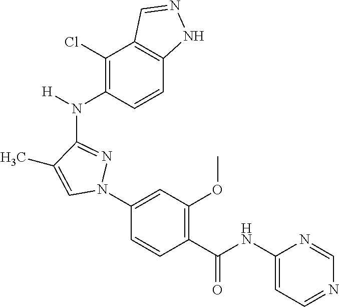 [SMILES: COc1cc(-n2cc(C)c(Nc3ccc4[nH]ncc4c3Cl)n2)ccc1C(=O)Nc1ccncn1]

A solution of 4-[3-[(4-chloro-1-tetrahydropyran-2-yl-indazol-5-yl)amino]-4-methyl-pyrazol-1-yl]-2-methoxy-N-pyrimidin-4-yl-benzamide (50 mg, 89.44 umol, 1 eq) in HCl/dioxane (1 mL) was stirred at 25° C. for 1 hr. The reaction mixture was filtered and the filter cake was concentrated in vacuo. The crude product was triturated with MeOH (3 mL) at 25° C. for 1 hr, then filtered and the filter cake was concentrated in vacuo to give 4-[3-[(4-chloro-1H-indazol-5-yl)amino]-4-methyl-pyrazol-1-yl]-2-methoxy-N-pyrimidin-4-yl-benzamide (14.1 mg, 24.91 umol, 27.85% yield, 90.336% purity, HCl) as a yellow solid. LC-MS (ES⁺, Method A), 0.48 min, m/z 475.4 [M+H]⁺*. ¹H NMR (400 MHz, DMSO-d6) 6=13.29-13.21 (m, 1H), 10.60 (s, 1H), 8.91 (s, 1H), 8.72 (d, J=5.6 Hz, 1H), 8.44 (s, 1H), 8.23 (d, J=5.6 Hz, 1H), 8.02 (s, 1H), 7.95 (d, J=8.8 Hz, 1H), 7.80 (d, J=9.2 Hz, 1H), 7.52-7.41 (m, 4H), 4.08 (s, 3H), 2.04 (s, 3H).

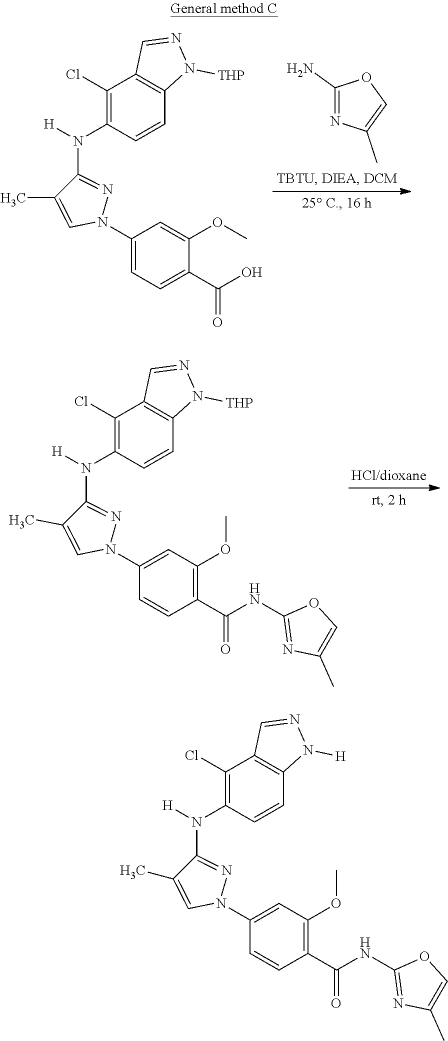

### Step 1: 4-(3-((4-chloro-1-(tetrahydro-2H-pyran-2-yl)-1H-indazol-5-yl)amino)-4-methyl-1H-pyrazol-1-yl)-2-methoxy-N-(4-methyloxazol-2-yl)benzamide

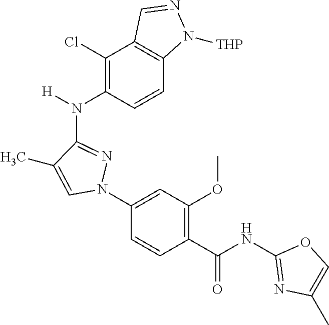

To a solution of 4-[3-[(4-chloro-1-tetrahydropyran-2-yl-indazol-5-yl)amino]-4-methyl-pyrazol-1-yl]-2-methoxy-benzoic acid (200 mg, 415.00 umol, 1 eq) in DCM (2 mL) was added TBTU (266.50 mg, 830.00 umol, 2 eq), 4-methyloxazol-2-amine (81.43 mg, 830.00 umol, 2 eq) and DIEA (160.91 mg, 1.24 mmol, 216.86 uL, 3 eq). The mixture was stirred at 25° C. for 16 hr. The reaction mixture was concentrated under reduced pressure to give a residue. The residue was purified by flash silica gel chromatography eluting with 0-100% EtOAc in Pet. Ether to give 4-[3-[(4-chloro-1-tetrahydropyran-2-yl-indazol-5-yl)amino]-4-methyl-pyrazol-1-yl]-2-methoxy-N-(4-methyloxazol-2-yl)benzamide (50 mg, 88.97 umol, 21.44% yield) as a yellow solid. LC-MS (ES⁺, Method A), 0.51 min, m/z 562.3 [M+H]⁺*.

### Example 92: 4-(3-((4-chloro-1H-indazol-5-yl)amino)-4-methyl-1H-pyrazol-1-yl)-2-methoxy-N-(4-methyloxazol-2-yl)benzamide

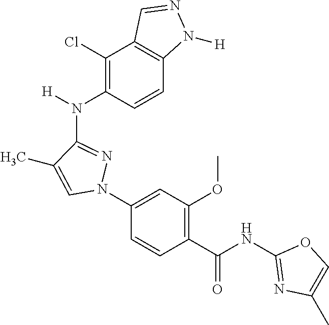

A mixture of 4-[3-[(4-chloro-1-tetrahydropyran-2-yl-indazol-5-yl)amino]-4-methyl-pyrazol-1-yl]-2-methoxy-N-(4-methyloxazol-2-yl)benzamide (50 mg, 88.97 umol, 1 eq) in HCl/dioxane (5 mL) was stirred at 25° C. for 16 hr. The reaction mixture was concentrated under reduced pressure to give a residue. The residue was purified by reversed-phase MPLC (TFA condition) to give 4-[3-[(4-chloro-1H-indazol-5-yl)amino]-4-methyl-pyrazol-1-yl]-2-methoxy-N-(4-methyloxazol-2-yl)benzamide (6.1 mg, 9.44 umol, 10.61% yield, 91.625% purity, TFA) as a yellow solid. LC-MS (ES⁺, Method A), 0.45 min, m/z 478.1 [M+H]⁺. ¹H NMR (400 MHz, DMSO-d₆) 6=13.23 (s, 1H), 10.71 (s, 1H), 8.41 (s, 1H), 8.01 (s, 1H), 7.76 (dd, J=5.2, 8.8 Hz, 2H), 7.61 (d, J=0.8 Hz, 1H), 7.49 (d, J=9.2 Hz, 1H), 7.44-7.35 (m, 3H), 3.96 (s, 3H), 2.11-1.95 (m, 6H).

Compounds prepared in a similar manner to that set out above are given below in

| Example | Structure | Method | LCMS | ¹H NMR |
|---|---|---|---|---|
| 93 | 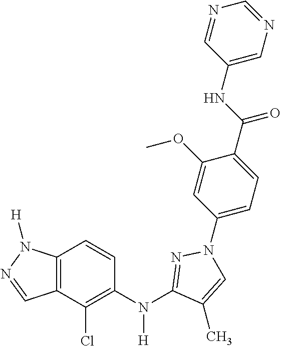 [SMILES: COc1cc(-n2cc(C)c(Nc3ccc4[nH]ncc4c3Cl)n2)ccc1C(=O)Nc1cncnc1] | A | LC-MS (ES⁺, Method A), 0.476 min, m/z 475.71 [M + H]⁺ | ¹H NMR (400 MHz, DMSO-d₆) δ = 10.31 (s, 1H), 9.16 (s, 2H), 8.92 (s, 1H), 8.43 (d, J = 0.8 Hz, 1H), 8.02 (d, J = 0.8 Hz, 1H), 7.83 (d, J = 8.4 Hz, 1H), 7.80-7.74 (m, 1H), 7.50 (d, J = 9.2 Hz, 1H), 7.46 (d, J = 1.6 Hz, 1H), 7.44- 7.40 (m, 1H), 4.02 (s, 3H), 2.03 (s, 3H). |
| 94 | 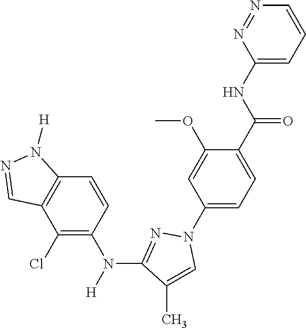 [SMILES: COc1cc(-n2cc(C)c(Nc3ccc4[nH]ncc4c3Cl)n2)ccc1C(=O)Nc1cccnn1] | A | LC-MS (ES⁺, Method A), 0.482 min, m/z 475.1 [M + H]⁺ | ¹H NMR (400 MHz, DMSO-6) δ = 13.29-13.19 (m, 1H), 10.89-10.80 (m, 1H), 9.05-8.98 (m, 1H), 8.53- 8.40 (m, 2H), 8.06-7.95 (m, 2H), 7.83-7.70 (m, 2H), 7.58-7.37 (m, 4H), 4.18-4.07 (m, 3H), 2.10-2.00 (m, 3H). |
| 95 | 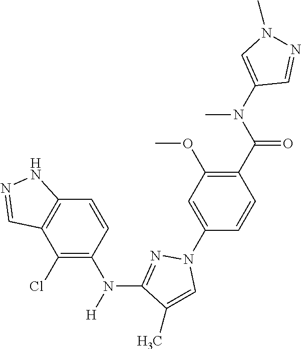 [SMILES: COc1cc(-n2cc(C)c(Nc3ccc4[nH]ncc4c3Cl)n2)ccc1C(=O)N(C)c1cnn(C)c1] | A | LC-MS (ES+, Method D), 0.44 min, m/z 491.2 [M + H]⁺. | ¹H NMR (400 MHz, DMSO-d6) δ = 8.22 (br s, 1H), 7.99 (s, 1H), 7.75 (d, J = 8.8 Hz, 1H), 7.47 (d, J = 8.8 Hz, 1H), 7.40-6.88 (m, 4H), 3.93-3.64 (m, 6H), 3.28-3.08 (m, 3H), 2.04 (s, 3H). |
| 96 | 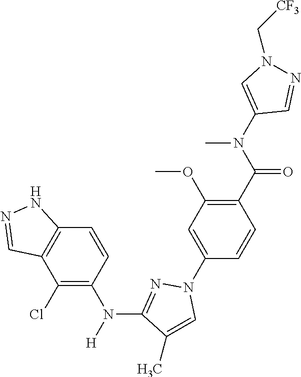 | A | LC-MS (ES+, Method D), 0.51 min, m/z 545.4 [M + H]⁺. | ¹H NMR (400 MHz, DMSO-d6) δ = 13.39-13.09 (m, 1H), 10.08-10.04 (m, 1H), 8.43-8.37 (m, 1H), 8.27- 8.21 (m, 1H), 8.02 (s, 1H), 7.83 (s, 3H), 7.50 (d, J = 8.8 Hz, 1H), 7.47-7.31 (m, 3H), 5.13 (q, J = 9.2 Hz, 2H), 4.06-3.94 (m, 3H), 3.45-3.32 (m, 1H), 3.39 (br s, 6H), 2.03 (s, 3H). |
| 97 | 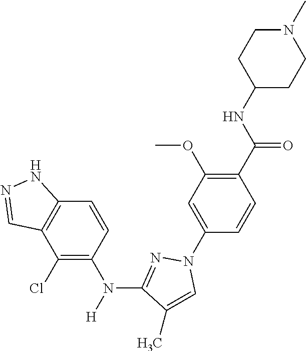 [SMILES: COc1cc(-n2cc(C)c(Nc3ccc4[nH]ncc4c3Cl)n2)ccc1C(=O)NC1CCN(C)CC1] | A | LC-MS (ES+, Method D), 0.38 min, m/z 494.4 [M + H]⁺. | ¹H NMR (400 MHz, DMSO-d6) δ = 9.67-9.17 (m, 1H), 8.28 (d, J = 0.8 Hz, 1H), 8.00 (d, J = 0.8 Hz, 1H), 7.92 (d, J = 5.2 Hz, 1H), 7.82-7.71 (m, 2H), 7.51-7.46 (m, 1H), 7.48 (dd, J = 0.8, 8.8 Hz, 1H), 7.41 (d, J = 2.0 Hz, 1H), 7.36 (dd, J = 2.0, 8.8 Hz, 1H), 7.07 (br s, 1H), 4.14-3.91 (m, 4H), 3.57-3.33 (m, 4H), 2.82 (s, 3H), 2.17-2.00 (m, 5H), 1.97-1.72 (m, 2H). |
| 98 | 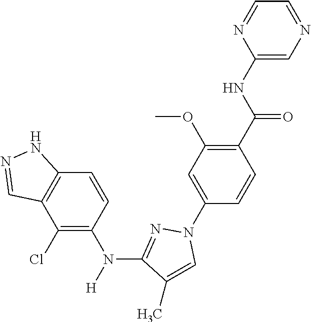 [SMILES: COc1cc(-n2cc(C)c(Nc3ccc4[nH]ncc4c3Cl)n2)ccc1C(=O)Nc1cnccn1] | B | LC-MS (ES⁺, Method A), 0.50 min, m/z 475.4 [M + H]⁺. | ¹H NMR (400 MHz, DMSO-d6) δ = 10.51 (s, 1H), 9.53- 9.47 (m, 1H), 8.49-8.38 (m, 3H), 8.05-7.94 (m, 2H), 7.79 (d, J = 8.8 Hz, 1H), 7.53-7.38 (m, 4H), 4.08 (s, 3H), 2.04 (s, 3H). |
| 99 | 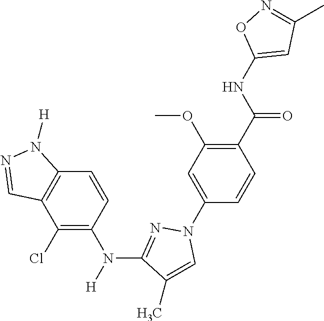 [SMILES: COc1cc(-n2cc(C)c(Nc3ccc4[nH]ncc4c3Cl)n2)ccc1C(=O)Nc1cc(C)no1] | B | LC-MS (ES⁺, Method A), 0.51 min, m/z 478.2 [M + H]⁺. | ¹H NMR (400 MHz, DMSO-d₆) δ = 13.24 (br s, 1H), 11.19 (s, 1H), 8.47-8.35 (m, 1H), 8.01 (s, 1H), 7.82- 7.71 (m, 2H), 7.50 (br d, J = 9.2 Hz, 1H), 7.46-7.33 (m, 3H), 6.27 (s, 1H), 3.98 (s, 3H), 2.21 (s, 3H), 2.03 (s, 3H). |
| 100 | 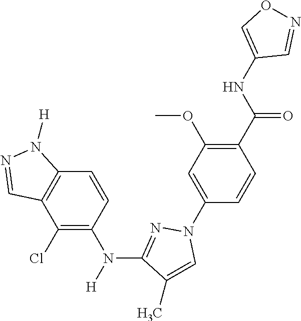 [SMILES: COc1cc(-n2cc(C)c(Nc3ccc4[nH]ncc4c3Cl)n2)ccc1C(=O)Nc1cnoc1] | B | LC-MS (ES⁺, Method A), 0.49 min, m/z 464.1 [M + H]⁺. | ¹H NMR (400 MHz, DMSO-d6) δ = 10.17 (s, 1H), 9.26- 9.21 (m, 1H), 8.80 (s, 1H), 8.41 (s, 1H), 8.02 (s, 1H), 7.85 (d, J = 8.4 Hz, 1H), 7.77 (d, J = 9.2 Hz, 1H), 7.50 (d, J = 8.8 Hz, 1H), 7.45-7.38 (m, 2H), 4.01 (s, 3H), 2.03 (s, 3H). |
| 101 | 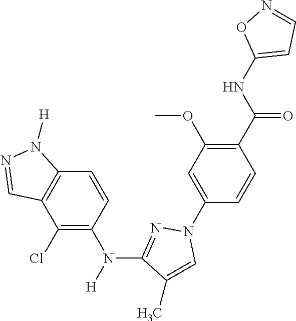 [SMILES: COc1cc(-n2cc(C)c(Nc3ccc4[nH]ncc4c3Cl)n2)ccc1C(=O)Nc1ccno1] | B | LC-MS (ES⁺, Method A), 0.51 min, m/z 464.1 [M + H]⁺. | ¹H NMR (400 MHz, DMSO-d₆) δ = 11.37-11.31 (m, 1H), 8.49 (d, J = 1.6 Hz, 1H), 8.41 (d, J = 0.8 Hz, 1H), 8.02 (d, J = 0.8 Hz, 1H), 7.77 (dd, J = 6.4, 8.8 Hz, 2H), 7.50 (dd, J = 0.8, 8.8 Hz, 1H), 7.45-7.35 (m, 3H), 6.37 (d, J = 1.6 Hz, 1H), 3.98 (s, 3H), 2.03 (s, 3H). |
| 102 | 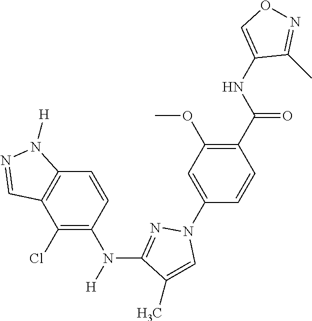 [SMILES: COc1cc(-n2cc(C)c(Nc3ccc4[nH]ncc4c3Cl)n2)ccc1C(=O)Nc1conc1C] | B | LC-MS (ES⁺, Method A), 0.52 min, m/z 478.0 [M + H]⁺. | ¹H NMR (400 MHz, DMSO-d6) δ = 9.75 (s, 1H), 9.13 (s, 1H), 8.41 (s, 1H), 8.02 (s, 1H), 7.88 (d, J = 8.4 Hz, 1H), 7.78 (d, J = 9.2 Hz, 1H), 7.52-7.45 (m, 2H), 7.42 (dd, J = 1.6, 8.4 Hz, 1H), 7.38 (s, 1H), 4.04 (s, 3H), 2.33 (s, 3H), 2.03 (s, 3H). |
| 103 | 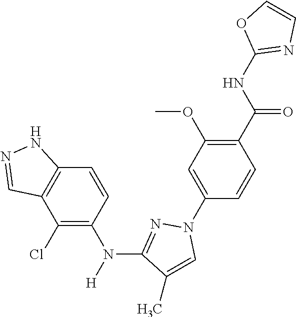 [SMILES: COc1cc(-n2cc(C)c(Nc3ccc4[nH]ncc4c3Cl)n2)ccc1C(=O)Nc1ncco1] | A | LC-MS (ES+, Method A), 0.55 min, m/z 464.1 [M + H]⁺. | ¹H NMR (400 MHz, DMSO-d6) δ = 13.39-13.08 (m, 1H), 10.86 (s, 1H), 8.46 (s, 1H), 8.06 (s, 1H), 7.98 (s, 1H), 7.85-7.70 (m, 2H), 7.55-7.46 (m, 1H), 7.45-7.34 (m, 3H), 7.22 (s, 1H), 3.93 (s, 3H), 2.03 (s, 3H). |
| 104 | 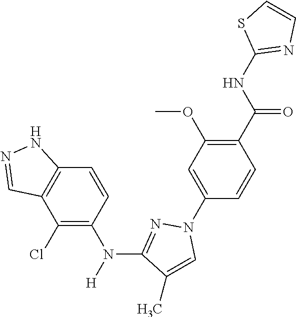 [SMILES: COc1cc(-n2cc(C)c(Nc3ccc4[nH]ncc4c3Cl)n2)ccc1C(=O)Nc1nccs1] | A | LC-MS (ES+, Method A), 0.48 min, m/z 480.2 [M + H]⁺. | ¹H NMR (400 MHz, DMSO-d6) δ = 11.59 (s, 1H), 8.44 (s, 1H), 8.03 (s, 1H), 7.87 (d, J = 8.8 Hz, 1H), 7.80 (d, J = 9.2 Hz, 1H), 7.54-7.50 (m, 2H), 7.47 (d, J = 2.0 Hz, 1H), 7.44 (dd, J = 2.0, 8.8 Hz, 2H), 7.29 (d, J = 3.2 Hz, 1H), 4.05 (s, 3H), 2.05 (s, 3H). |
| 105 | 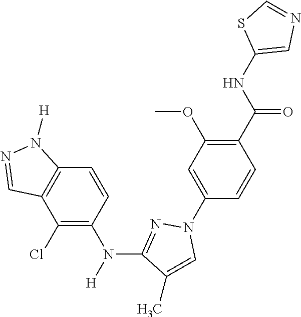 [SMILES: COc1cc(-n2cc(C)c(Nc3ccc4[nH]ncc4c3Cl)n2)ccc1C(=O)Nc1cncs1] | A | LC-MS (ES+, Method A), 0.48 min, m/z 480.3 [M + H]⁺. | ¹H NMR (400 MHz, DMSO-d6) δ = 11.19 (s, 1H), 8.62 (s, 1H), 8.43 (d, J = 0.8 Hz, 1H), 8.03 (d, J = 0.8 Hz, 1H), 7.84 (d, J = 0.8 Hz, 1H), 7.79 (t, J = 8.4 Hz, 2H), 7.51 (dd, J = 0.8, 8.8 Hz, 1H), 7.46 (d, J = 2.0 Hz, 1H), 7.44-7.37 (m, 2H), 4.01 (s, 3H), 2.04 (s, 3H). |
| 106 | 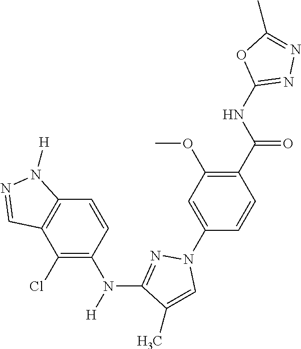 [SMILES: COc1cc(-n2cc(C)c(Nc3ccc4[nH]ncc4c3Cl)n2)ccc1C(=O)Nc1nnc(C)o1] | A | LC-MS (ES+, Method A), 0.56 min, m/z 478.9 [M + H]⁺. | ¹H NMR (400 MHz, DMSO-d6) δ = 13.24 (br s, 1H), 11.05 (s, 1H), 8.43 (s, 1H), 8.03 (s, 1H), 7.77 (t, J = 8.4 Hz, 2H), 7.51 (d, J = 8.8 Hz, 1H), 7.46-7.36 (m, 3H), 3.97 (s, 3H), 2.49 (s, 3H), 2.04 (s, 3H). |
| 107 | 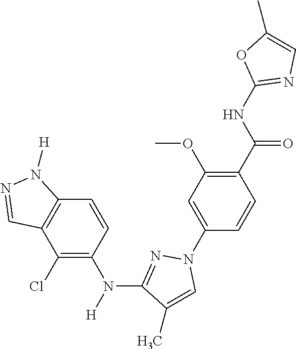 [SMILES: COc1cc(-n2cc(C)c(Nc3ccc4[nH]ncc4c3Cl)n2)ccc1C(=O)Nc1ncc(C)o1] | A | LC-MS (ES+, Method A), 0.47 min, m/z 478.2 [M + H]⁺. | ¹H NMR (400 MHz, DMSO-d6) δ = 10.75-10.56 (m, 1H), 8.42 (s, 1H), 8.02 (s, 1H), 7.81-7.72 (m, 2H), 7.50 (d, J = 8.8 Hz, 1H), 7.45-7.32 (m, 3H), 6.78 (d, J = 1.2 Hz, 1H), 3.97 (s, 3H), 2.29 (d, J = 0.8 Hz, 3H), 2.04 (s, 3H). |
| 108 | 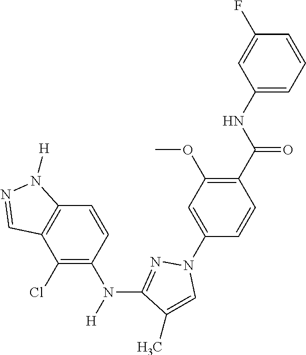 [SMILES: COc1cc(-n2cc(C)c(Nc3ccc4[nH]ncc4c3Cl)n2)ccc1C(=O)Nc1cccc(F)c1] | A | LC-MS (ES+, Method A), 0.53 min, m/z 491.3 [M + H]⁺. | ¹H NMR (400 MHz, DMSO-d6) δ = 13.23 (s, 1H), 10.20 (s, 1H), 8.39 (s, 1H), 8.07 (m, 1H), 7.81-7.72 (m, 3H), 7.53-7.47 (m, 2H), 7.45 (d, J = 1.6 Hz, 1H), 7.43-7.33 (m, 3H), 6.92 (dt, J = 2.4, 8.4 Hz, 1H), 4.06 (s, 3H), 2.06 (s, 3H). |
| 109 | 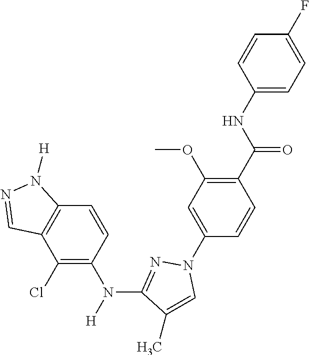 [SMILES: COc1cc(-n2cc(C)c(Nc3ccc4[nH]ncc4c3Cl)n2)ccc1C(=O)Nc1ccc(F)cc1] | A | LC-MS (ES+, Method A), 0.52 min, m/z 491.4 [M + H]⁺. | ¹H NMR (400 MHz, METHANOL-d4) δ = 8.16 (d, J = 0.8 Hz, 1H), 8.05-8.02 (m, 2H), 7.98-7.92 (m, 1H), 7.74- 7.66 (m, 2H), 7.56-7.52 (m, 1H), 7.52-7.47 (m, 1H), 7.44-7.39 (m, 1H), 7.18-7.08 (m, 2H), 4.13 (s, 3H), 2.12 (s, 3H). |
| 110 | 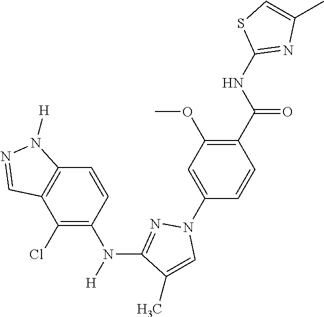 [SMILES: COc1cc(-n2cc(C)c(Nc3ccc4[nH]ncc4c3Cl)n2)ccc1C(=O)Nc1nc(C)cs1] | A | LC-MS (ES⁺, Method A), 0.522 min, m/z 494 [M + H]⁺. | ¹H NMR (400 MHz, DMSO-d6) δ = 13.30 (s, 1H), 11.56 (s, 1H), 8.47-8.40 (m, 1H), 8.05-7.97 (m, 1H), 7.90- 7.83 (m, 1H), 7.83-7.76 (m, 1H), 7.51 (d, J = 9.2 Hz, 1H), 7.48-7.37 (m, 3H), 6.82 (s, 1H), 4.04 (s, 3H), 2.29 (s, 3H), 2.04 (s, 3H). |
| 111 | 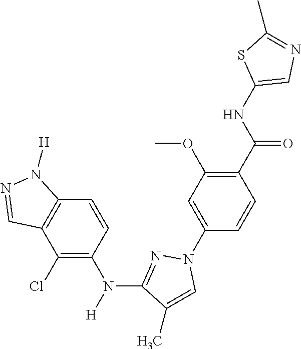 [SMILES: COc1cc(-n2cc(C)c(Nc3ccc4[nH]ncc4c3Cl)n2)ccc1C(=O)Nc1cnc(C)s1] | A | LC-MS (ES⁺, Method A), 0.440 min, m/z 494.11 [M + H]⁺. | ¹H NMR (400 MHz, DMSO-d6) δ = 11.27 (s, 1H), 8.52 (s, 1H), 8.02 (s, 1H), 7.84-7.75 (m, 2H), 7.72-7.64 (m, 1H), 7.57-7.28 (m, 5H), 4.01 (s, 3H), 2.64 (s, 3H), 2.03 (s, 3H). |
| 112 | 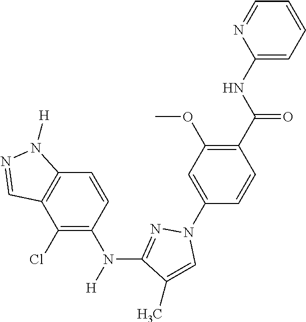 [SMILES: COc1cc(-n2cc(C)c(Nc3ccc4[nH]ncc4c3Cl)n2)ccc1C(=O)Nc1ccccn1] | A | LC-MS (ES⁺, Method A), 0.442 min, m/z 474 [M + H]⁺. | ¹H NMR (400 MHz, DMSO-d6) δ = 12.84 (s, 1H), 10.41 (s, 1H), 8.48-8.34 (m, 2H), 8.25 (d, J = 8.4 Hz, 1H), 8.06- 7.97 (m, 2H), 7.96-7.85 (m, 1H), 7.84-7.73 (m, 1H), 7.55-7.36 (m, 4H), 7.24-7.15 (m, 1H), 4.06 (s, 3H), 2.08 (s, 3H). |
| 113 | 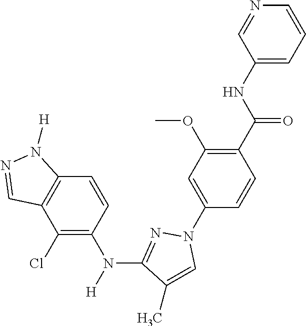 [SMILES: COc1cc(-n2cc(C)c(Nc3ccc4[nH]ncc4c3Cl)n2)ccc1C(=O)Nc1cccnc1] | A | LC-MS (ES⁺, Method A), 0.378 min, m/z 474 [M + H]⁺. | ¹H NMR (400 MHz, DMSO-d6) δ = 13.06 (s, 1H), 10.57 (s, 1H), 9.17 (s, 1H), 8.59-8.48 (m, 2H), 8.43 (s, 1H), 8.04-8.00 (m, 1H), 7.85-7.74 (m, 3H), 7.53-7.48 (m, 1H), 7.48-7.45 (m, 1H), 7.45-7.37 (m, 2H), 4.01 (s, 3H), 2.04 (s, 3H). |
| 114 | 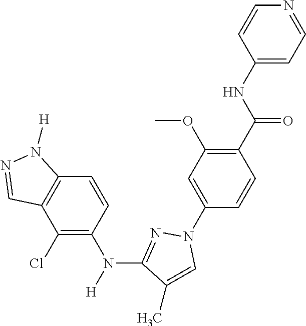 [SMILES: COc1cc(-n2cc(C)c(Nc3ccc4[nH]ncc4c3Cl)n2)ccc1C(=O)Nc1ccncc1] | A | LC-MS (ES⁺, Method A), 0.398 min, m/z 474 [M + H]⁺. | ¹H NMR (400 MHz, DMSO-d6) δ = 13.03 (s, 1H), 11.14 (s, 1H), 8.76-8.67 (m, 2H), 8.46-8.39 (m, 1H), 8.23- 8.15 (m, 2H), 8.04-7.98 (m, 1H), 7.81-7.71 (m, 2H), 7.52-7.47 (m, 1H), 7.45 (d, J = 1.9 Hz, 1H), 7.44-7.38 (m, 2H), 4.01 (s, 3H), 2.07 (s, 3H). |
| 115 | 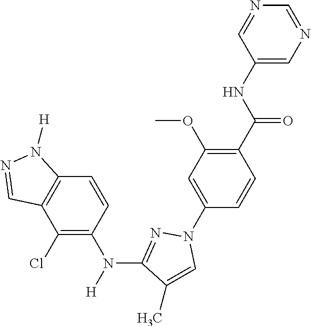 [SMILES: COc1cc(-n2cc(C)c(Nc3ccc4[nH]ncc4c3Cl)n2)ccc1C(=O)Nc1cncnc1] | A | LC-MS (ES⁺, Method A), 0.448 min, m/z 475.3 [M + H]⁺. | ¹H NMR (400 MHz, DMSO-d6) δ = 13.63 (s, 1H), 10.32 (s, 1H), 9.20-9.12 (m, 2H), 8.95-8.90 (m, 1H), 8.46- 8.41 (m, 1H), 8.02 (s, 1H), 7.83 (d, J = 8.4 Hz, 1H), 7.81- 7.76 (m, 1H), 7.53-7.49 (m, 1H), 7.46 (d, J = 1.6 Hz, 1H), 7.45-7.35 (m, 2H), 4.03 (s, 3H), 2.04 (s, 3H). |
| 116 | 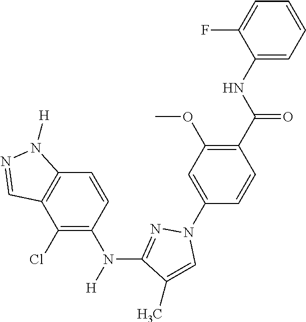 [SMILES: COc1cc(-n2cc(C)c(Nc3ccc4[nH]ncc4c3Cl)n2)ccc1C(=O)Nc1ccccc1F] | A | LC-MS (ES⁺, Method A), 0.520 min, m/z 490 [M + H]⁺. | ¹H NMR (400 MHz, DMSO-d6) δ = 13.20 (s, 1H), 10.12 (s, 1H), 8.46 (s, 1H), 8.27 (t, J = 7.6 Hz, 1H), 8.06-7.99 (m, 2H), 7.83-7.76 (m, 1H), 7.53-7.43 (m, 4H), 7.40 (s, 1H), 7.32 (dd, J = 8.0, 11.2 Hz, 1H), 7.25-7.13 (m, 3H), 4.10 (s, 3H), 2.04 (s, 3H). |

### General Route or the Synthesis of Examples 117-120

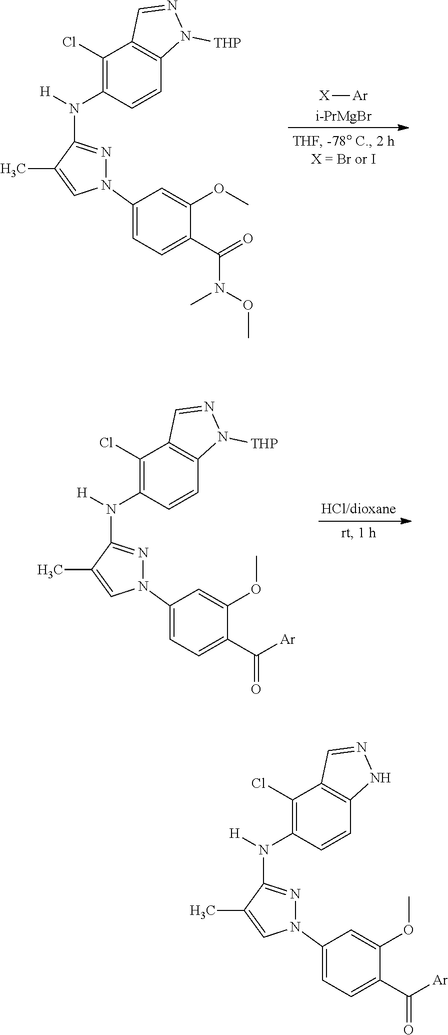

### Step 1: [4-[3-[(4-chloro-1-tetrahydropyran-2-yl-indazol-5-yl)amino]-4-methyl-pyrazol-1-yl]-2-methoxy-phenyl]-(1-methylpyrazol-4-yl)methanone

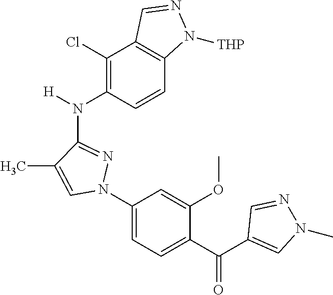

A mixture of 4-[3-[(4-chloro-1-tetrahydropyran-2-yl-indazol-5-yl)amino]-4-methyl-pyrazol-1-yl]-N,2-dimethoxy-N-methyl-benzamide (100 mg, 190.48 umol, 1 eq), 4-iodo-1-methyl-pyrazole (158.48 mg, 761.91 umol, 4 eq), i-PrMgBr (2 M, 476.19 uL, 5 eq) in THE (1 mL) was degassed and purged with N₂ for 3 times, and then the mixture was stirred at −78° C. for 2 hr under N₂ atmosphere. The reaction mixture was diluted with water 10 mL and extracted with EtOAc (15 mL*3). The combined organic layers were dried over Na₂SO₄, filtered and concentrated under reduced pressure to give [4-[3-[(4-chloro-1-tetrahydropyran-2-yl-indazol-5-yl)amino]-4-methyl-pyrazol-1-yl]-2-methoxy-phenyl]-(1-methylpyrazol-4-yl)methanone (100 mg, 183.14 umol, 96.15% yield) as yellow oil. LC-MS (ES⁺, Method A), 0.54 min, m/z 546.2 [M+H]⁺.

### Example 117: [4-[3-[(4-chloro-1H-indazol-5-yl)amino]-4-methyl-pyrazol-1-yl]-2-methoxy-phenyl]-(1-methylpyrazol-4-yl)methanone

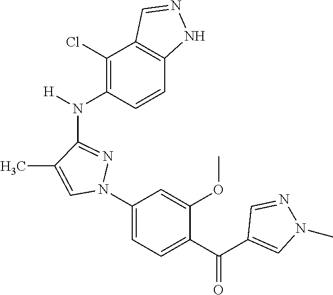

To a solution of [4-[3-[(4-chloro-1-tetrahydropyran-2 yl-indazol-5-yl)amino]-4-methyl-pyrazol-1-yl]-2-methoxy-phenyl]-(1-methylpyrazol-4-yl)methanone (100 mg, 183.14 umol, 1 eq) in HCl/dioxane (2 mL) was stirred at 25° C. for 2 hr. The reaction mixture was concentrated under reduced pressure to give a residue. The residue was purified by preparative HPLC (35-65% MeCN in H₂O) to afford [4-[3-[(4-chloro-1H-indazol-5-yl)amino]-4-methyl-pyrazol-1-yl]-2-methoxy-phenyl]-(1-methyl pyrazol-4-yl)methanone (9.4 mg, 19.35 umol, 10.57% yield, 95.083% purity) as a yellow solid. LC-MS (ES⁺, Method A), 0.42 min, m/z 462.4 [M+H]⁺. ¹H NMR (400 MHz, METHANOL-d4) δ 8.14 (d, J=0.8 Hz, 1H), 8.09 (s, 1H), 8.03 (s, 1H), 7.94 (d, J=9.0 Hz, 1H), 7.84 (s, 1H), 7.53-7.45 (m, 3H), 7.37 (dd, J=1.8, 8.4 Hz, 1H), 3.95 (s, 3H), 3.89 (s, 3H), 2.13 (s, 3H).

Compounds prepared in a similar manner to that set out above are given below in Table 22

| Example | Structure | LCMS | ¹H NMR |
|---|---|---|---|
| 118 | 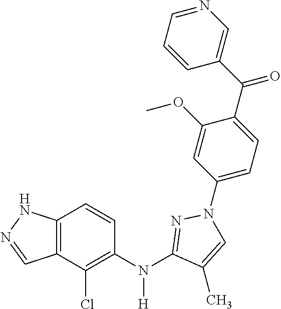 [SMILES: COc1cc(-n2cc(C)c(Nc3ccc4[nH]ncc4c3Cl)n2)ccc1C(=O)c1cccnc1] | LC-MS (ES⁺, Method A), 0.40 min, m/z 459.2 [M + H]⁺. | ¹H NMR (400 MHz, METHANOL-d4) δ = 8.84 (dd, J = 0.8, 2.4 Hz, 1H), 8.73 (dd, J = 1.6, 4.8 Hz, 1H), 8.20- 8.13 (m, 2H), 8.04 (s, 1H), 7.99 (d, J = 9.2 Hz, 1H), 7.64-7.55 (m, 2H), 7.52-7.42 (m, 3H), 3.78 (s, 3H), 2.14 (d, J = 0.8 Hz, 3H). |
| 119 | 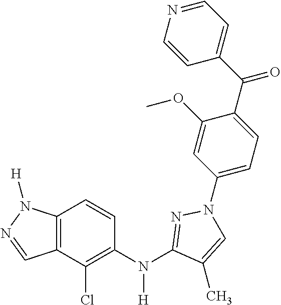 [SMILES: COc1cc(-n2cc(C)c(Nc3ccc4[nH]ncc4c3Cl)n2)ccc1C(=O)c1ccncc1] | LC-MS (ES⁺, Method A), 0.43 min, m/z 459.1 [M + H]⁺. | ¹H NMR (400 MHz, METHANOL-d4) δ 8.75-8.70 (m, 2H), 8.18 (d, J = 0.8 Hz, 1H), 8.05-7.98 (m, 2H), 7.66-7.61 (m, 3H), 7.51-7.48 (m, 2H), 7.44 (dd, J = 1.8, 8.4 Hz, 1H), 3.75 (s, 3H), 2.14 (s, 3H). |
| 120 | 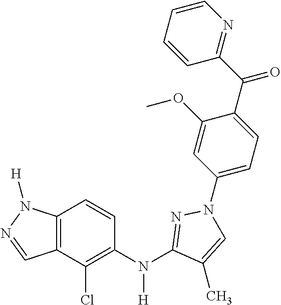 [SMILES: COc1cc(-n2cc(C)c(Nc3ccc4[nH]ncc4c3Cl)n2)ccc1C(=O)c1ccccn1] | LC-MS (ES⁺, Method A), 0.47 min, m/z 459.3 [M + H]⁺. | ¹H NMR (400 MHz, METHANOL-d4) δ = 8.60 (d, J = 4.4 Hz, 1H), 8.16 (d, J = 0.8 Hz, 1H), 8.07-7.96 (m, 3H), 7.88 (d, J = 8.0 Hz, 1H), 7.66 (d, J = 8.4 Hz, 1H), 7.63-7.57 (m, 1H), 7.50-7.40 (m, 3H), 3.70 (s, 3H), 2.13 (s, 3H). |

General method for the synthesis of Example 121:

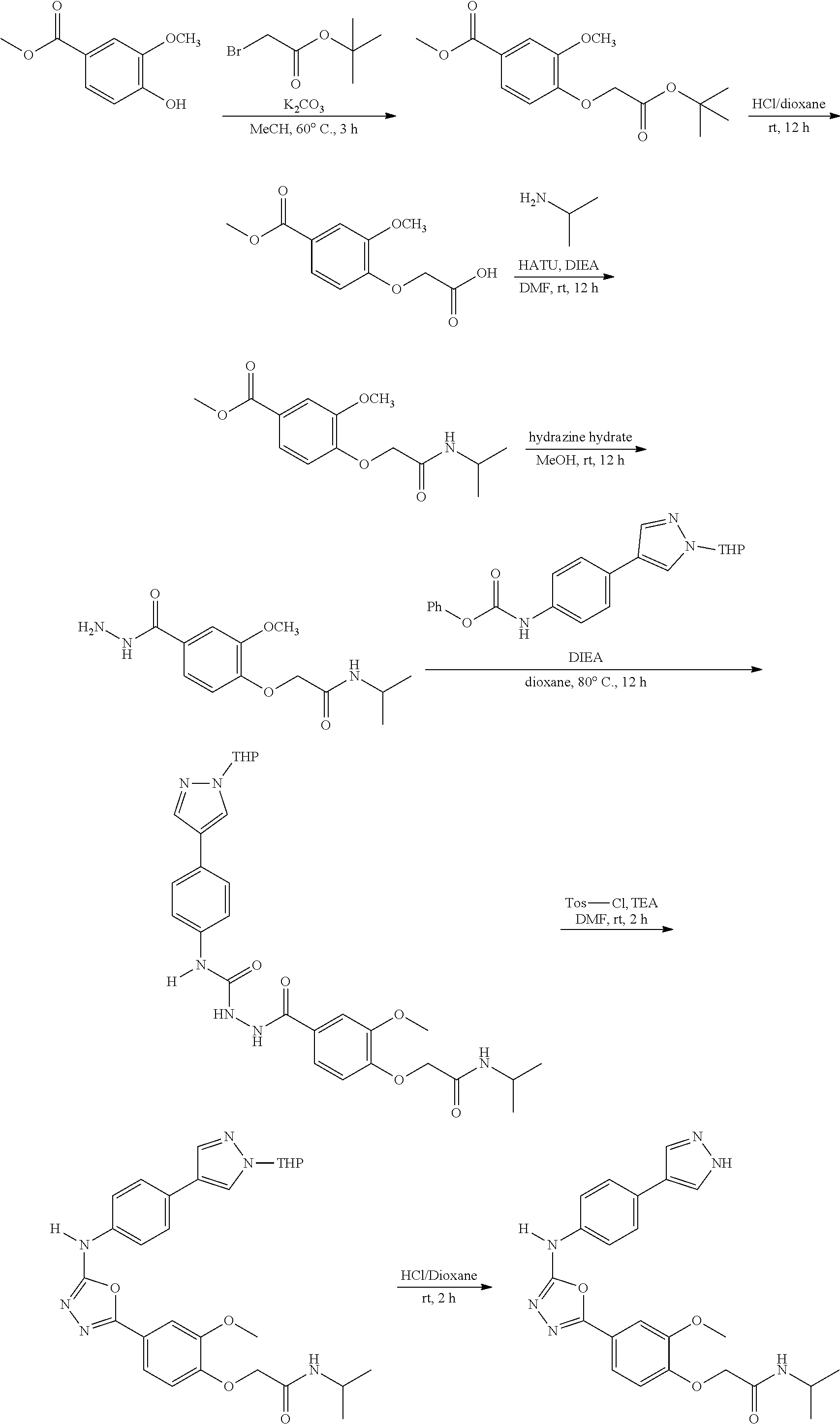

### Step 1: methyl 4-(2-tert-butoxy-2-oxo-ethoxy)-3-methoxy-benzoate

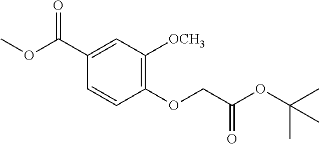 [SMILES: COC(=O)c1ccc(OCC(=O)OC(C)(C)C)c(OC)c1]

To a solution of methyl 4-hydroxy-3-methoxy-benzoate (3 g, 16.47 mmol), tert-butyl 2-bromoacetate (6.42 g, 32.94 mmol, 4.87 mL) in MeCN (15 mL) was added K₂CO₃ (4.55 g, 32.94 mmol). The mixture was stirred at 60° C. for 2 hr. The reaction mixture was diluted with water (50 mL) and extracted with EtOAc (3×50 mL). The combined organic layers dried over Na₂SO₄, filtered and concentrated under reduced pressure to give a residue. The residue was purified by flash silica gel chromatography eluting with 0-25% EtOAc in Pet. Ether to give methyl 4-(2-tert-butoxy-2-oxo-ethoxy)-3-methoxy-benzoate (4.5 g, 15.19 mmol, 92.22% yield) as a white solid. LC-MS (ES⁺, Method A), 0.47 min, m/z 296.3 [M+H]⁺.

### Step 2: 2-(2-methoxy-4-methoxycarbonyl-phenoxy)acetic acid

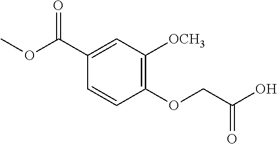 [SMILES: COC(=O)c1ccc(OCC(=O)O)c(OC)c1]

A mixture of methyl 4-(2-tert-butoxy-2-oxo-ethoxy)-3-methoxy-benzoate (4.5 g, 15.19 mmol) in HCl/dioxane (30 mL) was stirred at 25° C. for 2 hr. The reaction mixture concentrated under reduced pressure to give 2-(2-methoxy-4-methoxycarbonyl-phenoxy)acetic acid (4 g, crude) as a white solid, which was used directly in the next step without further purification. LC-MS (ES⁺, Method A), 0.33 min, m/z 241.0 [M+H]⁺.

### Step 3: methyl 4-[2-(isopropylamino)-2-oxo-ethoxy]-3-methoxy-benzoate

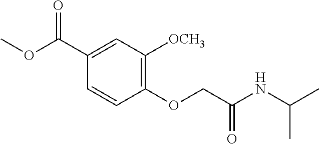 [SMILES: COC(=O)c1ccc(OCC(=O)NC(C)C)c(OC)c1]

To a solution of 2-(2-methoxy-4-methoxycarbonyl-phenoxy)acetic acid (4 g, 16.65 mmol), propan-2-amine (1.97 g, o 33.30 mmol, 2.86 mL, 2 eq) in DMF (30 mL) was added HATU (9.50 g, 24.98 mmol) and DIEA (10.76 g, 83.26 mmol, 14.50 mL). The mixture was stirred at 25° C. for 2 hr. The reaction mixture was diluted with water (50 mL) and extracted with EtOAc (3×50 mL). The combined organic layers dried over Na₂SO₄, filtered and concentrated under reduced pressure to give a residue. The residue was purified by flash silica gel chromatography eluting with 0-50% EtOAc in Pet. Ether to give methyl 4-[2-(isopropylamino)-2-oxo-ethoxy]-3-methoxy-benzoate (6 g, crude) as a white solid. LC-MS (ES⁺, Method A), 0.37 min, m/z 282.0 [M+H]⁺.

### Step 4: 2-[4-(hydrazinecarbonyl)-2-methoxy-phenoxy]-N-isopropyl-acetamide

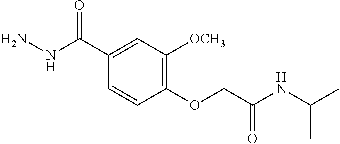 [SMILES: COc1cc(C(=O)NN)ccc1OCC(=O)NC(C)C]

To a solution of methyl 4-[2-(isopropylamino)-2-oxo-ethoxy]-3-methoxy-benzoate (3 g, 10.66 mmol) in MeOH (30 mL) was added hydrazine hydrate (5.1 g, 101.88 mmol, 4.95 mL). The mixture was stirred at 25° C. for 2 hr. The reaction mixture was concentrated under reduced pressure to give 2-[4-(hydrazinecarbonyl)-2-methoxy-phenoxy]-N-isopropyl-acetamide (1.5 g, 5.33 mmol, 50.00% yield) as a white solid. LC-MS (ES⁺, Method A), 0.25 min, m/z 281.9 [M+H]⁺.

### Step 5: N-isopropyl-2-[2-methoxy-4-[[[4-(1-tetrahydropyran-2-ylpyrazol-4-yl)phenyl]carbamoylamino]carbamoyl]phenoxy]acetamide

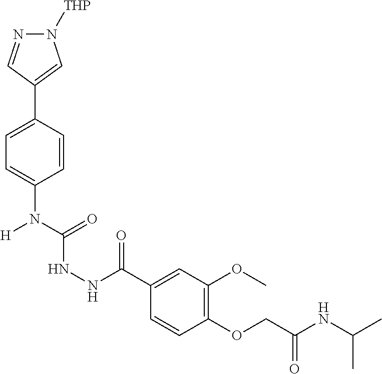

To a solution of 2-[4-(hydrazinecarbonyl)-2-methoxy-phenoxy]-N-isopropyl-acetamide (500 mg, 1.78 mmol, 1 eq) and phenyl N-[4-(1-tetrahydropyran-2-ylpyrazol-4-yl)phenyl]carbamate (646.87 mg, 1.78 mmol, 1 eq) in dioxane (5 mL) was added DIEA (690.14 mg, 5.34 mmol, 930.11 uL, 3 eq). The mixture was stirred at 80° C. for 16 hr. The reaction mixture was concentrated under reduced pressure to give a residue. The residue was purified by preparative HPLC (25-55% MeCN in H₂O) to afford N-isopropyl-2-[2-methoxy-4-[[[4-(1-tetrahydropyran-2-ylpyrazol-4-yl)phenyl]carbamoylamino]carbamoyl] phenoxy]acetamide (400 mg, 726.47 umol, 40.81% yield) as a white solid. LC-MS (ES⁺, Method A), 0.40 min, m/z 551.2 [M+H]⁺*.

### Step 6: N-isopropyl-2-[2-methoxy-4-[5-[4-(1-tetrahydropyran-2-ylpyrazol-4-yl) anilino]-1,3,4-oxadiazol-2-yl]phenoxy]acetamide

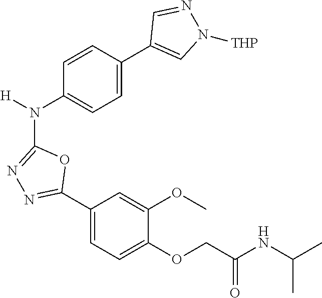

To a solution of N-isopropyl-2-[2-methoxy-4-[[[4-(1-tetrahydropyran-2-ylpyrazol-4-yl)phenyl] carbamoy 1 amino]carbamoyl]phenoxy] acetamide (350.00 mg, 635.66 umol, 1 eq) in DCM (3 mL) was added TosCl (302.97 mg, 1.59 mmol, 2.5 eq) and TEA (321.61 mg, 3.18 mmol, 442.38 uL, 5 eq). The mixture was stirred at 25° C. for 2 hr. The reaction mixture was filtered and concentrated under reduced pressure to give a residue. The residue was purified by flash silica gel chromatography eluting with 0-20% MeOH in DCM to afford N-isopropyl-2-[2-methoxy-4-[5-[4-(1-tetrahydropyran-2-ylpyrazol-4-yl)anilino]-1,3,4-oxadiazol-2-yl]phenoxy]acetamide (300 mg, 563.29 umol, 88.61% yield) as a yellow solid. LC-MS (ES⁺, Method A), 0.44 min, m/z 533.3 [M+H]⁺*.

### Example 121: N-isopropyl-2-[2-methoxy-4-[5-[4-(1H-pyrazol-4-yl)anilino]-1,3,4-oxadiazol-2-yl]phenoxy]acetamide

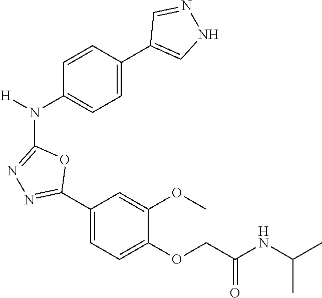 [SMILES: COc1cc(-c2nnc(Nc3ccc(-c4cn[nH]c4)cc3)o2)ccc1OCC(=O)NC(C)C]

To a solution of N-isopropyl-2-[2-methoxy-4-[5-[4-(1-tetrahydropyran-2-ylpyrazol-4-yl)anilino]-1,3,4-oxadiazol-2-yl]phenoxy]acetamide (150 mg, 281.64 umol, 1 eq) in HCl/dioxane (5 mL) was stirred at 25° C. for 1 hr. The reaction mixture was filtered and concentrated under reduced pressure to give a residue. The residue was purified by preparative HPLC (10-40% MeCN in H₂O) to afford N-isopropyl-2-[2-methoxy-4-[5-[4-(1H-pyrazol-4-yl)anilino]-1,3,4-oxadiazol-2-yl]phenoxy] acetamide (87.6 mg, 189.25 umol, 67.20% yield, 96.89% purity) as an off-white solid. LC-MS (ES⁺, Method A), 0.39 min, m/z 449.1 [M+H]⁺*. ¹H NMR (400 MHz, MeOH-d4) δ 8.08 (s, 2H), 7.67-7.58 (m, 5H), 7.55 (dd, J=1.6, 8.4 Hz, 1H), 7.16 (d, J=8.4 Hz, 1H), 4.60 (s, 2H), 4.16-4.05 (m, 1H), 4.01 (s, 3H), 1.22 (d, J=6.4 Hz, 6H).

General method for the synthesis of Example 122-125

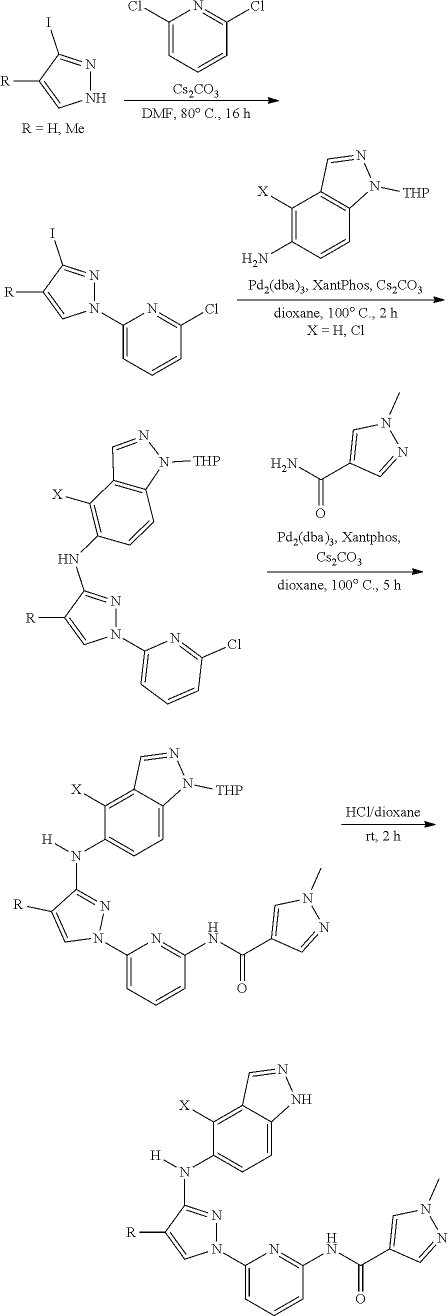

### Step 1: 2-chloro-6-(3-iodo-1H-pyrazol-1-yl)pyridine

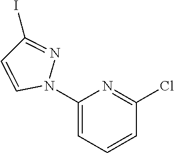 [SMILES: Clc1cccc(-n2ccc(I)n2)n1]

To a solution of 3-iodo-1H-pyrazole (1 g, 5.16 mmol, 1 eq) and 2,6-dichloropyridine (991.82 mg, 6.70 mmol, 1.3 eq) in DMF (10 mL) was added Cs₂CO₃ (5.04 g, 15.47 mmol, 3 eq). The mixture was stirred at 80° C. for 16 hr. The mixture was pour into water (100 ml), filtered and the solid was concentrated in vacuum to give a residue. The residue was triturated with MeOH at 25° C. for 60 min to give 2-chloro-6-(3-iodopyrazol-1-yl)pyridine (2.7 g, 8.84 mmol, 85.72% yield) as a white solid. LC-MS (ES⁺, Method A), 0.488 min, m/z 305.9 [M+H]⁺.

### Step 2: N-(1-(6-chloropyridin-2-yl)-1H-pyrazol-3-yl)-1-(tetrahydro-2H-pyran-2-yl)-1H-indazol-5-amine

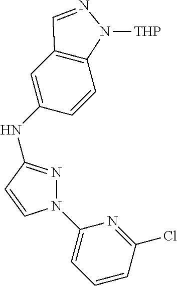

A mixture of 2-chloro-6-(3-iodopyrazol-1-yl)pyridine (300 mg, 981.99 umol, 1 eq), 1-tetrahydropyran-2-ylindazol-5-amine (213.35 mg, 981.99 umol, 1 eq), Pd₂(dba)₃ (89.92 mg, 98.20 umol, 0.1 eq), Xantphos (113.64 mg, 196.40 umol, 0.2 eq) and Cs₂CO₃ (639.90 mg, 1.96 mmol, 2 eq) in dioxane (3 mL) was degassed and purged with N₂ for 3 times, and then the mixture was stirred at 100° C. for 2 hr under N₂ atmosphere. The reaction mixture was concentrated under reduced pressure to give a residue. The residue was purified by preparative HPLC (55-85% MeCN in H₂O) to give N-[1-(6-chloro-2-pyridyl)pyrazo]-3-yl]-1-tetrahydropyran-2-yl-indazol-5-amine (140 mg, 354.56 umol, 36.11% yield) as a yellow solid. LC-MS (ES⁺, Method A), 0.533 min, m/z 395.1 [M+H]⁺.

### Step 3: 1-methyl-N-(6-(3-((1-(tetrahydro-2H-pyran-2-yl)-1H-indazol-5-yl)amino)-1H-pyrazol-1-yl)pyridin-2-yl)-1H-pyrazole-4-carboxamide

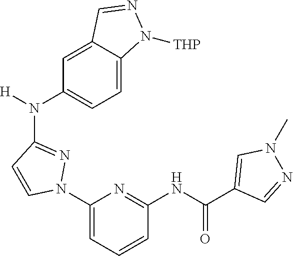

A mixture of N-[1-(6-chloro-2-pyridyl)pyrazo]-3-yl]-1-tetrahydropyran-2-yl-indazol-5-amine (140 mg, 354.56 umol, 1 eq), 1-methylpyrazole-4-carboxamide (48.80 mg, 390.01 umol, 1.1 eq), Pd₂(dba)₃ (32.47 mg, 35.46 umol, 0.1 eq), Xantphos (41.03 mg, 70.91 umol, 0.2 eq) and Cs₂CO₃ (231.04 mg, 709.12 umol, 2 eq) in dioxane (2 mL) was degassed and purged with N₂ for 3 times, and then the mixture was stirred at 100° C. for 16 hr under N₂ atmosphere. The reaction mixture was concentrated under reduced pressure to give a residue. The residue was purified by preparative-TLC (EtOAc) to afford 1-methyl-N-[6-[3-[(1-tetrahydropyran-2-ylindazol-5-yl)amino]pyrazol-1-yl]-2-pyridyl]pyrazole-4-carboxamide (150 mg, 310.22 umol, 87.50% yield) as a yellow solid. LC-MS (ES⁺, Method A), 0.568 min, m/z 498.3 [M+H]⁺.

### Example 122: N-(6-(3-((1H-indazol-5-yl)amino)-1H-pyrazol-1-yl)pyridin-2-yl)-1-methyl-1H-pyrazole-4-carboxamide

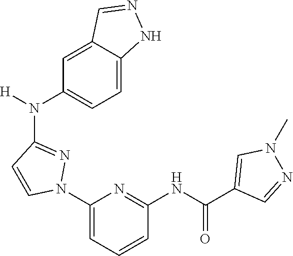 [SMILES: Cn1cc(C(=O)Nc2cccc(-n3ccc(Nc4ccc5[nH]ncc5c4)n3)n2)cn1]

A mixture of 1-methyl-N-[6-[3-[(1-tetrahydropyran-2-ylindazol-5-yl)amino]pyrazol-1-yl]-2-pyridyl]pyrazole-4-carboxamide (150 mg, 310.22 umol, 1 eq) in HCl/dioxane (4 M, 4 mL) was stirred at 25° C. for 4 hr. The reaction mixture was concentrated under reduced pressure to give a residue. The crude product was triturated with EtOAc and MeOH at 25° C. for 30 min to give N-[6-[3-(1H-indazol-5-ylamino)pyrazo]-1-yl]-2-pyridyl]-1-methyl-pyrazole-4-carboxamide (107.7 mg, 240.15 umol, 77.41% yield, 97.191% purity, HCl) as a yellow solid. LC-MS (ES⁺, Method A), 0.395 min, m/z 400.1 [M+H]⁺. 1H NMR (400 MHz, DMSO-d6) 6=10.32 (s, 1H), 8.48 (s, 1H), 8.43 (d, J=2.8 Hz, 1H), 8.20-8.13 (m, 2H), 8.02-7.93 (m, 3H), 7.61-7.53 (m, 1H), 7.45 (d, J=8.8 Hz, 1H), 7.33 (dd, J=2.0, 8.8 Hz, 1H), 6.18 (d, J=2.8 Hz, 1H), 3.90 (s, 3H).

Compounds prepared in a similar manner to that set out above are given below in Table 23.

| Example | Structure | LCMS | ¹H NMR |
|---|---|---|---|
| 123 | 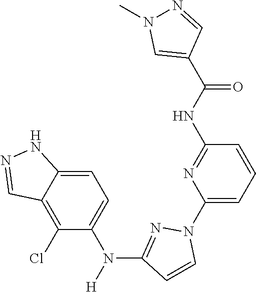 [SMILES: Cn1cc(C(=O)Nc2cccc(-n3ccc(Nc4ccc5[nH]ncc5c4Cl)n3)n2)cn1] | LC-MS (ES⁺, Method A), 0.438 min, m/z 434.1 [M + H]⁺. | ¹H NMR (400 MHz, DMSO-d6) δ = 10.32 (s, 1H), 8.47 (s, 1H), 8.42 (d, J = 2.8 Hz, 1H), 8.28-8.20 (m, 1H), 8.14 (s, 1H), 8.01 (s, 1H), 7.98-7.88 (m, 2H), 7.54 (d, J = 9.2 Hz, 1H), 7.44 (d, J = 7.2 Hz, 1H), 6.31 (d, J = 2.4 Hz, 1H), 3.92-3.87 (m, 3H). |
| 124 | 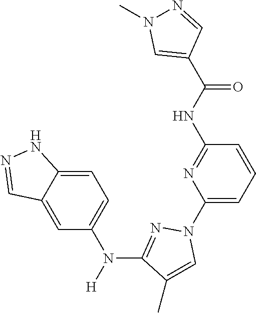 [SMILES: Cc1cn(-c2cccc(NC(=O)c3cnn(C)c3)n2)nc1Nc1ccc2[nH]ncc2c1] | LC-MS (ES⁺, Method A), 0.412 min, m/z 414.2 [M + H]⁺ | ¹H NMR (400 MHz, DMSO-d6) δ = 10.23 (s, 1H), 8.74- 8.50 (m, 2H), 8.47 (s, 1H), 8.27 (s, 2H), 8.15 (s, 1H), 7.99 (d, J = 0.8 Hz, 1H), 7.94-7.87 (m, 2H), 7.58- 7.48 (m, 2H), 7.45 (d, J = 8.8 Hz, 1H), 3.91 (s, 3H), 2.13 (s, 3H). |
| 125 | 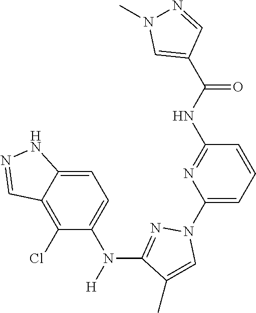 [SMILES: Cc1cn(-c2cccc(NC(=O)c3cnn(C)c3)n2)nc1Nc1ccc2[nH]ncc2c1Cl] | LC-MS (ES⁺, Method A), 0.464 min, m/z 448.1 [M + H]⁺. | ¹H NMR (400 MHz, DMSO-d6) δ = 10.30 (s, 1H), 8.49 (s, 1H), 8.30 (s, 1H), 8.15 (s, 1H), 8.01 (s, 1H), 7.92- 7.80 (m, 3H), 7.51 (d, J = 8.8 Hz, 1H), 7.33 (d, J = 7.6 Hz, 1H), 3.90 (s, 3H), 2.05 (s, 3H). |

General method for the synthesis of Example 126-128

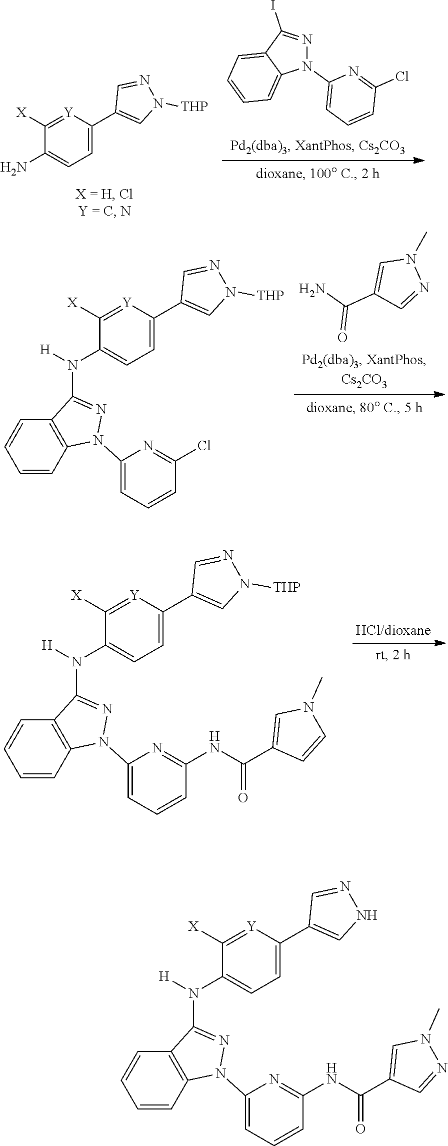

### Step 1: Coupling of 4-(1-tetrahydropyran-2-ylpyrazol-4-yl)aniline and 1-(6-chloro-2-pyridyl)-3-iodo-indazole

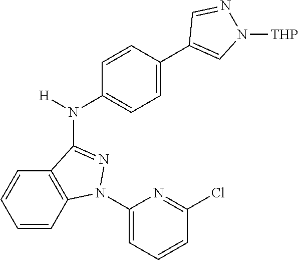

To a solution of 4-(1-tetrahydropyran-2-ylpyrazol-4-yl)aniline (300 mg, 1.23 mmol, 1 eq) and 1-(6-chloro-2-pyridyl)-3-iodo-indazole (438.42 mg, 1.23 mmol, 1 eq) in dioxane (3 mL) was added Cs₂CO₃ (803.49 mg, 2.47 mmol, 2 eq), Pd₂(dba)₃ (35.45 mg, 61.65 umol, 0.05 eq) and Xantphos (71.35 mg, 123.30 umol, 0.1 eq). The mixture was stirred at 100° C. for 16 hr. The mixture was concentrated under reduced pressure affording the residue. The residue was purified by preparative HPLC (80-100% MeCN in H₂O) to give 1-(6-chloro-2-pyridyl)-N-[4-(1-tetrahydropyran-2-ylpyrazol-4-yl)phenyl]indazol-3-amine (300 mg, 637.01 umol, 51.66% yield) as a white solid. LC-MS (ES⁺, Method A), 0.755 min, m/z 471.1 [M+H]⁺.

### Step 2: 1-methyl-N-(6-(3-((4-(1-(tetrahydro-2H-pyran-2-yl)-1H-pyrazol-4-yl)phenyl)amino)-1H-indazol-1-yl)pyridin-2-yl)-1H-pyrazole-4-carboxamide

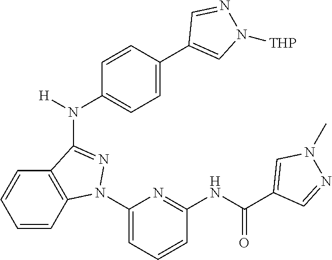

To a solution of 1-(6-chloro-2-pyridyl)-N-[4-(1-tetrahydropyran-2-ylpyrazol-4-yl)phenyl]indazol-3-amine (50 mg, 106.17 umol, 1 eq) and 1-methylpyrazole-4-carboxamide (19.93 mg, 159.25 umol, 1.5 eq) in dioxane (1 mL) was added Pd₂(dba)₃ (3.05 mg, 5.31 umol, 0.05 eq) and Xantphos (6.14 mg, 10.62 umol, 0.1 eq) and Cs₂CO₃ (69.18 mg, 212.34 umol, 2 eq). The mixture was stirred at 100° C. for 16 hr. The mixture was filtered, and concentrated under reduced pressure affording the residue. The residue was purified by prep-TLC (SiO₂, Petroleum ether/Ethyl acetate=1/2) to give 1-methyl-N-[6-[3-[4-(1-tetrahydropyran-2-ylpyrazol-4-yl)anilino]indazol-1-yl]-2-pyridyl]pyrazole-4-carboxamide (30 mg, 53.61 umol, 50.49% yield) as a yellow solid. LC-MS (ES⁺, Method A), 0.530 min, m/z 560.3 [M+H]⁺.

### Example 126: N-(6-(3-((6-(1H-pyrazol-4-yl)pyridin-3-yl)amino)-1H-indazol-1-yl)pyridin-2-yl)-1-methyl-1H-pyrazole-4-carboxamide

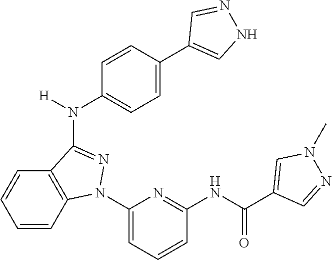 [SMILES: Cn1cc(C(=O)Nc2cccc(-n3nc(Nc4ccc(-c5cn[nH]c5)cc4)c4ccccc43)n2)cn1]

A mixture of 1-methyl-N-[6-[3-[[6-(1-tetrahydropyran-2-ylpyrazol-4-yl)-3-pyridyl]amino]indazol-1-yl]-2-pyridyl]pyrazole-4-carboxamide (40 mg, 71.35 umol, 1 eq) in HCl/dioxane (4 M, 1 mL) was stirred at 25° C. for 2 hr under N₂ atmosphere. The mixture was concentrated under reduced pressure affording the residue. The residue was purified by preparative HPLC (13-43% MeCN in H₂O) to give 1-methyl-N-[6-[3-[[6-(1H-pyrazol-4-yl)-3-pyridyl]amino]indazol-1-yl]-2-pyridyl]pyrazole-4-carboxamide (2.3 mg, 4.10 umol, 5.74% yield, 93.1% purity, FA) as a yellow solid. LC-MS (ES⁺, Method A), 0.445 min, m/z 477.3 [M+H]⁺. 1H NMR (400 MHz, DMSO-d6) 6=10.33 (s, 1H), 9.68-9.59 (m, 1H), 9.18-9.11 (m, 1H), 9.07-8.99 (m, 1H), 8.51 (s, 1H), 8.34 (d, J=6.4 Hz, 2H), 8.23-8.04 (m, 4H), 7.99-7.86 (m, 2H), 7.73 (d, J=8.8 Hz, 1H), 7.67-7.58 (m, 2H), 7.35 (t, J=7.2 Hz, 1H), 3.94 (s, 3H).

Compounds prepared in a similar manner to that set out above are given below in Table 24.

| Example | Structure | LCMS | ¹H NMR |
|---|---|---|---|
| 127 | 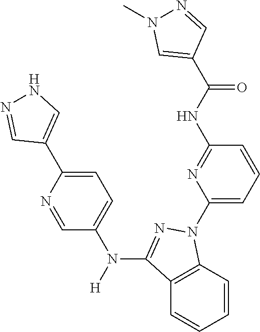 [SMILES: Cn1cc(C(=O)Nc2cccc(-n3nc(Nc4ccc(-c5cn[nH]c5)nc4)c4ccccc43)n2)cn1] | LC-MS (ES⁺, Method A), 0.445 min, m/z 477.3 [M + H]⁺.. | ¹H NMR (400 MHz, DMSO-d6) δ = 10.33 (s, 1H), 9.68- 9.59 (m, 1H), 9.18-9.11 (m, 1H), 9.07-8.99 (m, 1H), 8.51 (s, 1H), 8.34 (br d, J = 6.4 Hz, 2H), 8.23-8.04 (m, 4H), 7.99-7.86 (m, 2H), 7.73 (br d, J = 8.8 Hz, 1H), 7.67-7.58 (m, 2H), 7.35 (br t, J = 7.2 Hz, 1H), 3.94 (s, 3H). |
| 128 | 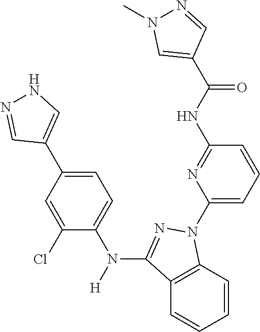 [SMILES: Cn1cc(C(=O)Nc2cccc(-n3nc(Nc4ccc(-c5cn[nH]c5)cc4Cl)c4ccccc43)n2)cn1] | LC-MS (ES⁺, Method A), 0.506 min, m/z 510.2 [M + H]⁺. | ¹H NMR (400 MHz, DMSO-d6) δ = 10.32 (s, 1H), 9.13 (d, J = 8.4 Hz, 1H), 8.56-8.42 (m, 2H), 8.24 (m, 2H), 8.16-8.07 (m, 2H), 7.99 (m, 1H), 7.90 (q, J = 8.4 Hz, 2H), 7.80 (d, J = 2.0 Hz, 1H), 7.68-7.58 (m, 2H), 7.53 (d, J = 7.2 Hz, 1H), 7.32 (t, J = 7.6 Hz, 1H), 3.94 (s, 3H). |

General method for the synthesis of Example 129-131:

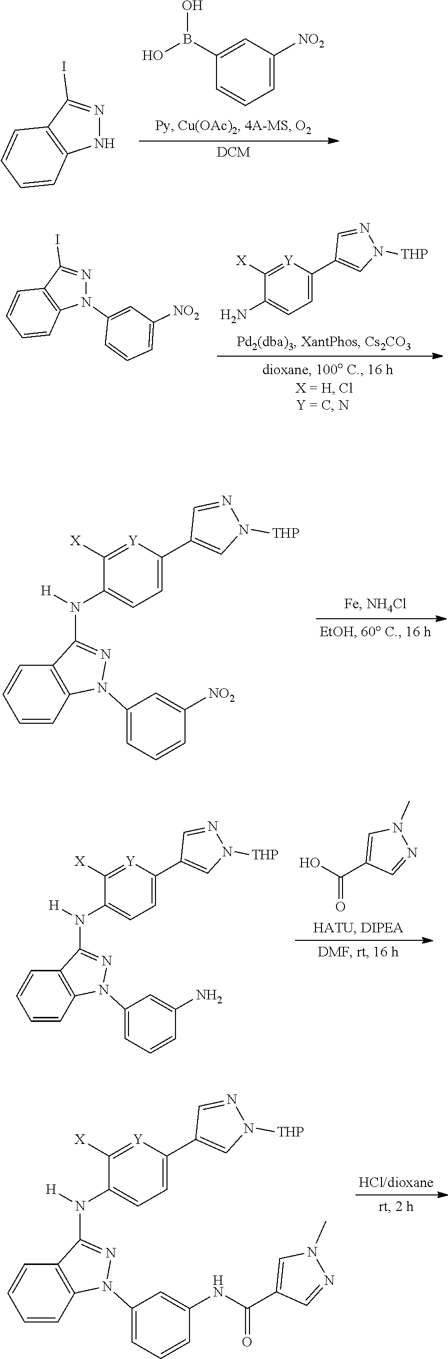

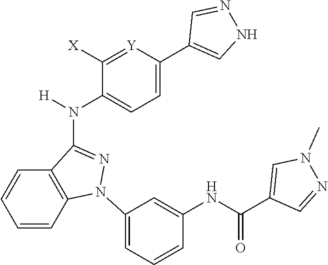

### Step 1: 3-iodo-1-(3-nitrophenyl)indazole

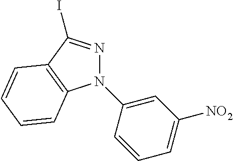 [SMILES: O=[N+]([O-])c1cccc(-n2nc(I)c3ccccc32)c1]

A mixture of 3-iodo-1H-indazole (5 g, 20.49 mmol, 1 eq), (3-nitrophenyl)boronic acid (3.42 g, 20.49 mmol, 1 eq), Cu(OAc)₂ (7.44 g, 40.98 mmol, 2 eq), Py (2.43 g, 30.73 mmol, 2.48 mL, 1.5 eq) and 4A MS (2.5 g, 1.00 eq), boric acid (2.53 g, 40.98 mmol, 2 eq) in MeCN (50 mL) was degassed and purged with N₂ for 3 times, and then the mixture was stirred at 60° C. for 16 hr under N₂ atmosphere. The reaction mixture was filtered to remove 4 A MS. The residue was concentrated under reduced pressure to give a residue. The residue was purified by flash silica gel chromatography eluting with 0-17% EtOAc in Pet. Ether to give 3-iodo-1-(3-nitrophenyl)indazole (2.5 g, 6.85 mmol, 33.42% yield) as a yellow solid. LC-MS (ES⁺, Method A), 0.56 min, m/z 365.9 [M+H]⁺.

### Step 2: 1-(3-nitrophenyl)-N-[4-(1-tetrahydropyran-2-ylpyrazol-4-yl)phenyl]indazol-3-amine

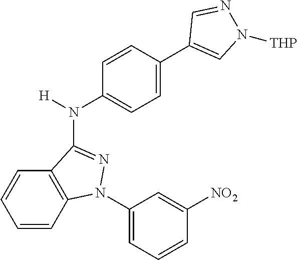

A mixture of 3-iodo-1-(3-nitrophenyl)indazole (500 mg, 1.37 mmol, 1 eq), 4-(1-tetrahydropyran-2-ylpyrazol-4-yl)aniline (333.18 mg, 1.37 mmol, 1 eq), Pd₂(dba)₃ (125.40 mg, 136.94 umol, 0.1 eq), Xantphos (79.24 mg, 136.94 umol, 0.1 eq) and Cs₂CO₃ (892.35 mg, 2.74 mmol, 2 eq) in dioxane (5 mL) was degassed and purged with N₂ for 3 times, and then the mixture was stirred at 100° C. for 16 hr under N₂ atmosphere. The reaction mixture was concentrated under reduced pressure to give a residue. The residue was purified by flash silica gel chromatography eluting with 0-50% EtOAc in Pet. Ether to affrod 1-(3-nitrophenyl)-N-[4-(1-tetrahydropyran-2-ylpyrazol-4-yl)phenyl]indazol-3-amine (500 mg, 1.04 mmol, 75.99% yield) as a brown oil. LC-MS (ES⁺, Method A), 0.58 min, m/z 481.0 [M+H]⁺.

### Step 3: 1-(3-aminophenyl)-N-[4-(1-tetrahydropyran-2-ylpyrazol-4-yl)phenyl]indazol-3-amine

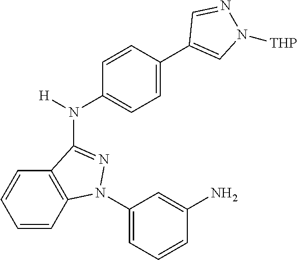

To a solution of 1-(3-nitrophenyl)-N-[4-(1-tetrahydropyran-2-ylpyrazol-4-yl)phenyl]indazol-3-amine (200 mg, 416.22 umol, 1 eq) in EtOH (2 mL) and H₂O (0.2 mL) was added Fe (116.22 mg, 2.08 mmol, 5 eq) and NH₄Cl (111.32 mg, 2.08 mmol, 5 eq). The mixture was stirred at 60° C. for 16 hr. The reaction mixture was filtered to remove the insoluble and the filter liquor was concentrated in vacuo to give a residue. The residue was diluted with H₂O 10 mL and extracted with EtOAc (10 mL*3). The combined organic layers were washed with brine (20 mL), dried over Na₂SO₄, filtered and concentrated under reduced pressure to give the crude product 1-(3-aminophenyl)-N-[4-(1-tetrahydropyran-2-ylpyrazol-4-yl)phenyl]indazol-3-amine (180 mg, crude) as brown solid. LC-MS (ES⁺, Method A), 0.51 min, m/z 451.4 [M+H]⁺.

### Step 4: 1-methyl-N-[3-[3-[4-(1-tetrahydropyran-2-ylpyrazol-4-yl)anilino]indazol-1-yl]phenyl]pyrazole-4-carboxamide

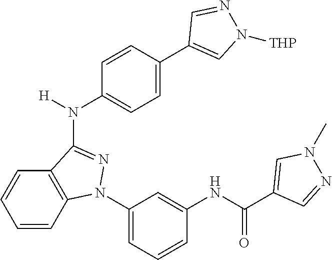

To a solution of 1-(3-aminophenyl)-N-[4-(1-tetrahydropyran-2-ylpyrazol-4-yl)phenyl]indazol-3-amine (180 mg, 399.53 umol, 1 eq) and 1-_{H} methylpyrazole-4-carboxylic acid (100.77 mg, 799.05 umol, 2 eq) in DMF (3 mL) was added HATU (227.87 mg, 599.29 umol, 1.5 eq) and DIEA (154.91 mg, 1.20 mmol, 208.77 uL, 3 eq). The mixture was stirred at 25° C. for 2 hr. The reaction mixture was diluted with water (10 mL) and extracted with EtOAc (15 mL*3). The combined organic layers were dried over Na₂SO₄, filtered and concentrated under reduced pressure to give a residue. The residue was purified by prep-TLC (SiO₂, DCM: MeOH=10:1) to afford 1-methyl-N-[3-[3-[4-(1-tetrahydropyran-2-ylpyrazol-4-yl)anilino]indazol-1-yl]phenyl]pyrazole-4-carboxamide (50 mg, 89.50 umol, 22.40% yield) as a yellow solid. LC-MS (ES⁺, Method A), 0.52 min, m/z 559.2 [M+H]⁺.

### Example 129: 1-methyl-N-[3-[3-[4-(1H-pyrazol-4-yl)anilino]indazol-1-yl]phenyl]pyrazole-4-carboxamide

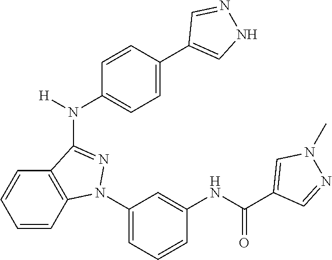 [SMILES: Cn1cc(C(=O)Nc2cccc(-n3nc(Nc4ccc(-c5cn[nH]c5)cc4)c4ccccc43)c2)cn1]

To a solution of 1-methyl-N-[3-[3-[4-(1-tetrahydropyran-2-ylpyrazol-4-yl)anilino]indazol-1-yl]phenyl]pyrazole-4-carboxamide (50 mg, 89.50 umol, 1 eq) in HCl/dioxane (3 mL) was stirred at 25° C. for 0.5 hr. The reaction mixture was concentrated under reduced pressure to give a residue. The residue was purified by preparative HPLC (35-65% MeCN in H₂O) to afford 1-methyl-N-[3-[3-[4-(1H-pyrazol-4-yl)anilino]indazol-1-yl]phenyl]pyrazole-4-carboxamide (6.8 mg, 14.20 umol, 15.86% yield, 99.073% purity) as a white solid. LC-MS (ES⁺, Method A), 0.47 min, m/z 475.0 [M+H]⁺.¹H NMR (400 MHz, METHANOL-d4) 6=8.32-8.30 (m, 1H), 8.26 (s, 1H), 8.11 (s, 1H), 8.04 (d, J=8.0 Hz, 1H), 7.96-7.91 (m, 2H), 7.87-7.84 (m, 1H), 7.84-7.84 (m, 1H), 7.87-7.84 (m, 1H), 7.85 (d, J=8.8 Hz, 1H), 7.63-7.59 (m, 1H), 7.59-7.50 (m, 5H), 7.64-7.48 (m, 1H), 7.23 (t, J=7.6 Hz, 1H), 4.00 (s, 3H).

Compounds prepared in a similar manner to that set out above are given below in Table 25.

| Example | Structure | LCMS | ¹H NMR |
|---|---|---|---|
| 130 | 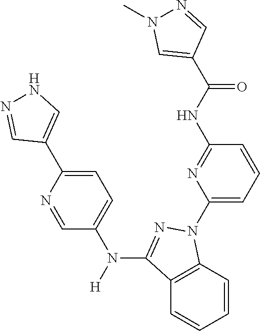 [SMILES: Cn1cc(C(=O)Nc2cccc(-n3nc(Nc4ccc(-c5cn[nH]c5)nc4)c4ccccc43)n2)cn1] | LC-MS (ES⁺, Method A), 0.445 min, m/z 477.3 [M + H]⁺. | ¹H NMR (400 MHz, METHANOL-d4) δ 9.66 (d, J = 2.0 Hz, 1H), 8.58-8.49 (m, 2H), 8.38 (s, 2H), 8.28 (s, 1H), 8.22 (d, J = 8.8 Hz, 1H), 8.12 (s, 1H), 8.07 (d, J = 8.0 Hz, 1H), 7.98 (d, J = 8.4 Hz, 1H), 7.64-7.53 (m, 3H), 7.44 (br d, J = 8.4 Hz, 1H), 7.37-7.30 (m, 1H), 4.01 (s, 3H). |
| 131 | 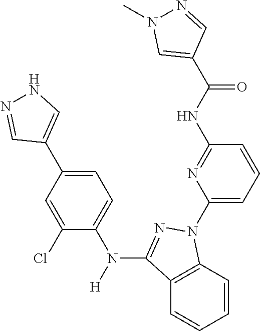 [SMILES: Cn1cc(C(=O)Nc2cccc(-n3nc(Nc4ccc(-c5cn[nH]c5)cc4Cl)c4ccccc43)n2)cn1] | LC-MS (ES⁺, Method A), 0.50 min, m/z 509.0 [M + H]⁺. | ¹H NMR (400 MHz, METHANOL-d4) δ = 8.35-8.28 (m, 2H), 8.27-8.16 (m, 3H), 8.10 (s, 1H), 7.98-7.88 (m, 2H), 7.75 (d, J = 2.0 Hz, 1H), 7.62-7.52 (m, 5H), 7.28 (t, J = 7.6 Hz, 1H), 3.99 (s, 3H). |

General method for the synthesis of Example 132-134:

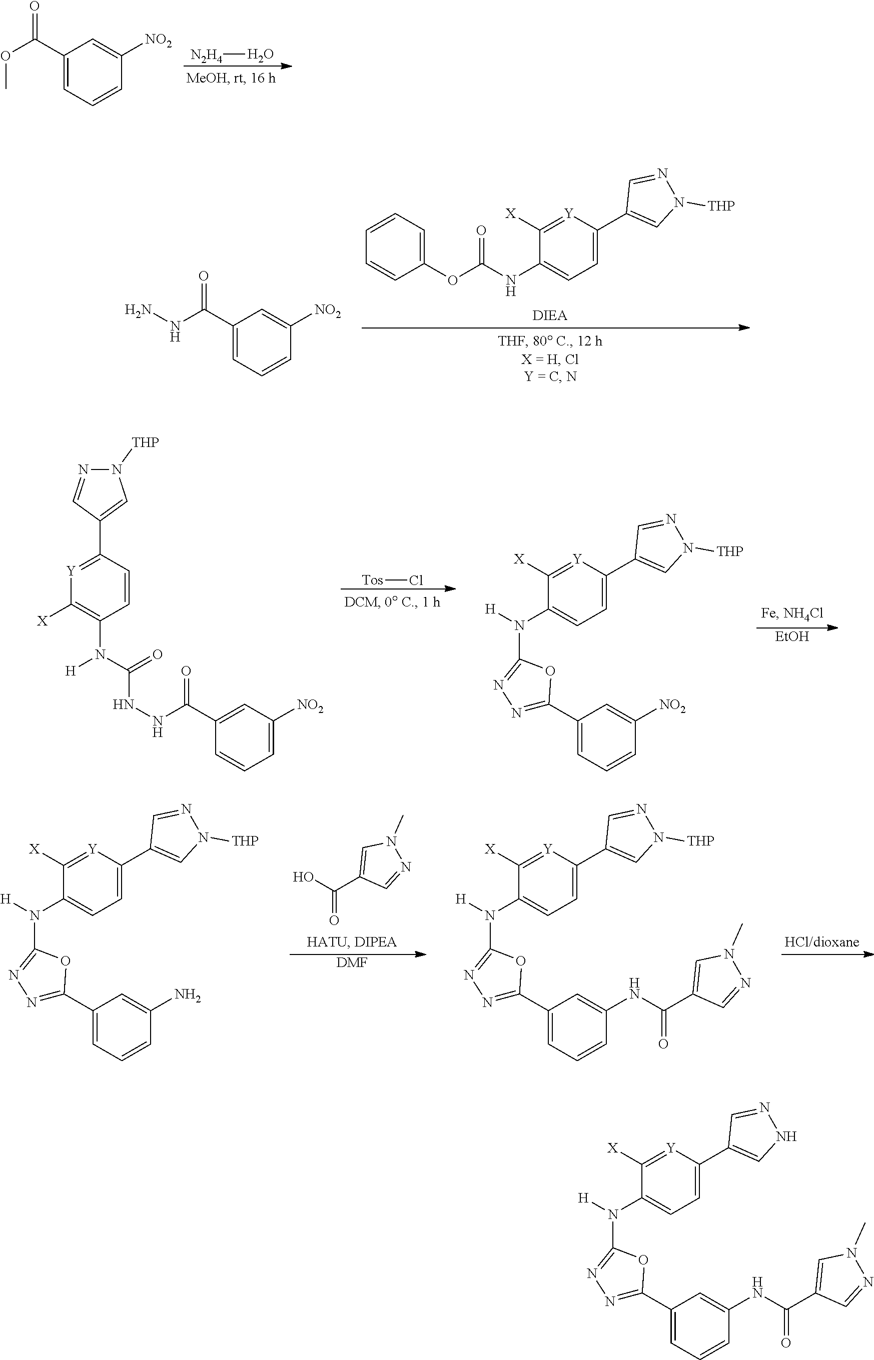

### Step 1: 3-nitrobenzohydrazide

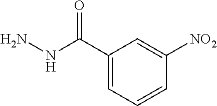 [SMILES: NNC(=O)c1cccc([N+](=O)[O-])c1]

To a solution of methyl 3-nitrobenzoate (5 g, 27.60 mmol, 1 eq) in MeOH (50 mL) was added N₂H₄-H₂O (10.620 g, 212.14 mmol, 10.31 mL, 7.69 eq). The mixture was stirred at 25° C. for 16 hr. The reaction mixture was filtered and the filter cake was washed with MeOH (20 mL) and dried over vacuum to give 3-nitrobenzohydrazide (4.2 g, 23.19 mmol, 84.00% yield) as a white solid. LC-MS (ES⁺, Method A), 0.157 min, m/z 182.1 [M+H]⁺.

### Step 2: 1-[(3-nitrobenzoyl)amino]-3-[4-(1-tetrahydropyran-2-ylpyrazol-4-yl)phenyl]urea

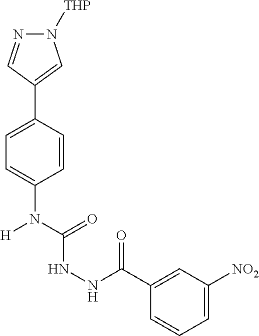

To a solution of phenyl N-[4-(1-tetrahydropyran-2-ylpyrazol-4-yl)phenyl]carbamate (900 mg, 2.48 mmol, 1 eq) in dioxane (10 mL) was added DIEA (960.23 mg, 7.43 mmol, 1.29 mL, 3 eq) and 3-nitrobenzohydrazide (448.62 mg, 2.48 mmol, 1 eq). The mixture was stirred at 80° C. for 16 hr. The mixture was concentrated under reduced pressure affording the residue. The residue was purified by column chromatography eluting with 0-100% EtOAc in Pet. Ether to give 1-[(3-nitrobenzoyl)amino]-3-[4-(1-tetrahydropyran-2-ylpyrazol-4-yl)phenyl]urea (600 mg, 1.33 mmol, 53.79% yield) as a yellow solid. LC-MS (ES⁺, Method A), 0.408 min, m/z 451.2 [M+H]⁺.

### Step 3: 5-(3-nitrophenyl)-N-(4-(1-(tetrahydro-2H-pyran-2-yl)-1H-pyrazol-4-yl)phenyl)-1,3,4-oxadiazol-2-amine

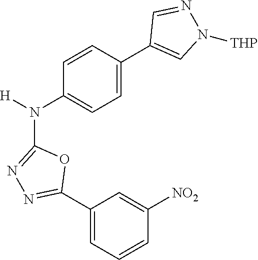

To a solution of 1-[(3-nitrobenzoyl)amino]-3-[4-(1-tetrahydropyran-2-ylpyrazol-4-yl)phenyl]urea (500 mg, 1.11 mmol, 1 eq) in DMF (5 mL) was added TosCl (529.05 mg, 2.78 mmol, 2.5 eq) and TEA (561.60 mg, 5.55 mmol, 772.50 uL, 5 eq). The mixture was stirred at 25° C. for 1 hr. The mixture was concentrated under reduced pressure affording the residue. The residue was purified by preparative HPLC (46-76% MeCN in H₂O) to give 5-(3-nitrophenyl)-N-[4-(1-tetrahydropyran-2-ylpyrazol-4-yl)phenyl]-1,3,4-oxadiazol-2-amine (220 mg, 508.75 umol, 45.83% yield) as a yellow solid. LC-MS (ES⁺, Method A), 0.419 min, m/z 433.3 [M+H]⁺.

### Step 4: 5-(3-aminophenyl)-N-(4-(1-(tetrahydro-2H-pyran-2-yl)-1H-pyrazol-4-yl)phenyl)-1,3,4-oxadiazol-2-amine

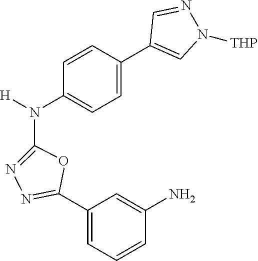

To a solution of 5-(3-nitrophenyl)-N-[4-(1-tetrahydropyran-2-ylpyrazol-4-yl)phenyl]-1,3,4-oxadiazol-2-amine (50 mg, 115.63 umol, 1 eq) in EtOH (1 mL) and H₂O (0.1 mL) was added Fe (32.29 mg, 578.13 umol, 5 eq) and NH₄Cl (30.92 mg, 578.13 umol, 5 eq). The mixture was stirred at 60° C. for 2 hr. The mixture was filtered, and concentrated under reduced pressure affording the residue, then, quenched by slow addition of H₂O (0.5 mL). The resulting mixture was transferred to a separatory funnel, and the aqueous layer mixture was extracted with ethyl acetate (1 mL×3).

The combined organic layers were washed with brine (1 mL), dried over anhydrous sodium sulfate, filtered, and concentrated under reduced pressure to give 5-(3-aminophenyl)-N-[4-(1-tetrahydropyran-2-ylpyrazol-4-yl)phenyl]-1,3,4-oxadiazol-2-amine (50 mg, crude) as a white solid. LC-MS (ES⁺, Method A), 0.377 min, m/z 403.1 [M+H]⁺.

### Step 5: 1-methyl-N-(3-(5-((4-(1-(tetrahydro-2H-pyran-2-yl)-1H-pyrazol-4-yl)phenyl)amino)-1,3,4-oxadiazol-2-yl)phenyl)-1H-pyrazole-4-carboxamide

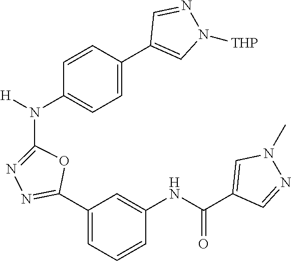

To a solution of 5-(3-aminophenyl)-N-[4-(1-tetrahydropyran-2-ylpyrazol-4-yl)phenyl]-1,3,4-oxadiazol-2-amine (90 mg, 223.63 umol, 1 eq) in DMF (2 mL) was added HATU (127.55 mg, 335.45 umol, 1.5 eq) and DIEA (144.51 mg, 1.12 mmol, 194.76 uL, 5 eq) and 1-methylpyrazole-4-carboxylic acid (56.41 mg, 447.26 umol, 2 eq). The mixture was stirred at 25° C. for 16 hr. The mixture was concentrated under reduced pressure affording the residue. The residue was purified by prep-HPLC (column: Phenomenex luna C18 150*25 mm* 10 um; mobile phase: [water(FA)-ACN]; B %: 30%-60%, 10 min) to give 1-methyl-N-[3-[5-[4-(1-tetrahydropyran-2-ylpyrazol-4-yl)anilino]-1,3,4-oxadiazol-2-yl]phenyl]pyrazole-4-carboxamide (80 mg, 156.69 umol, 70.07% yield) as a yellow solid. LC-MS (ES⁺, Method A), 0.393 min, m/z 511.3 [M+H]⁺.

### Example 132: N-(3-(5-((4-(1H-pyrazol-4-yl)phenyl)amino)-1,3,4-oxadiazol-2-yl)phenyl)-1-methyl-1H-pyrazole-4-carboxamide

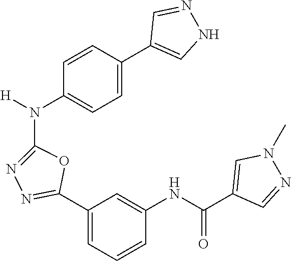 [SMILES: Cn1cc(C(=O)Nc2cccc(-c3nnc(Nc4ccc(-c5cn[nH]c5)cc4)o3)c2)cn1]

A solution of 1-methyl-N-[3-[5-[4-(1-tetrahydropyran-2-ylpyrazol-4-yl)anilino]-1,3,4-oxadiazol-2-yl]phenyl]pyrazole-4-carboxamide (40 mg, 78.35 umol, 1 eq) in HCl/dioxane (1 mL) was stirred at 25° C. for 1 hr. The mixture was concentrated under reduced pressure affording the residue. The residue was triturated with EtOAc at 25° C. for 30 min to give 1-methyl-N-[3-[5-[4-(1H-pyrazol-4-yl)anilino]-1,3,4-oxadiazol-2-yl]phenyl]pyrazole-4-carboxamide (57.3 mg, 116.61 umol, 74.42% yield, 94.2% purity, HCl) as a yellow solid. LC-MS (ES⁺, Method A), 0.437 min, m/z 427.3 [M+H]⁺. ¹H NMR (400 MHz, DMSO-d6) 6=10.76 (s, 1H), 10.15 (s, 1H), 8.47 (s, 1H), 8.39 (s, 1H), 8.11-8.06 (m, 3H), 7.88 (d, J=8.4 Hz, 1H), 7.64-7.49 (m, 7H), 3.91 (s, 3H).

Compounds prepared in a similar manner to that set out above are given below in Table 26.

| Example | Structure | LCMS | ¹H NMR |
|---|---|---|---|
| 133 | 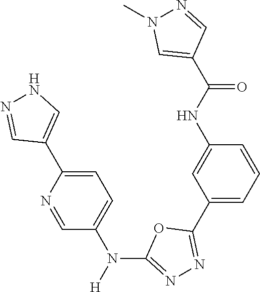 [SMILES: Cn1cc(C(=O)Nc2cccc(-c3nnc(Nc4ccc(-c5cn[nH]c5)nc4)o3)c2)cn1] | LC-MS (ES+, Method A), 0.315 min, m/z 428.1 [M + H]+. | ¹H NMR (400 MHz, METHANOL-d4) δ = 9.19 (d, J = 2.4 Hz, 1H), 8.53 (t, J = 1.6 Hz, 1H), 8.46 (dd, J = 2.8, 9.2 Hz, 1H), 8.42 (s, 2H), 8.29 (d, J = 9.2 Hz, 1H), 8.23 (s, 1H), 8.06 (s, 1H), 7.80-7.73 (m, 2H), 7.58-7.51 (m, 1H), 3.97 (s, 3H). |
| 134 | 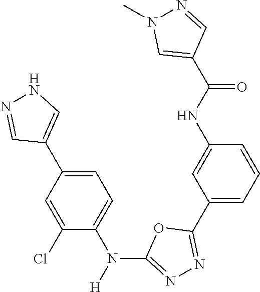 [SMILES: Cn1cc(C(=O)Nc2cccc(-c3nnc(Nc4ccc(-c5cn[nH]c5)cc4Cl)o3)c2)cn1] | LC-MS (ES⁺, Method A), 0.401 min, m/z 461.2 [M + H]⁺. | ¹H NMR (400 MHz, DMSO-d6) δ = 10.10 (s, 1H), 8.41 (s, 1H), 8.36 (s, 1H), 8.15 (s, 2H), 8.06 (s, 1H), 7.99 (d, J = 8.4 Hz, 1H), 7.89 (d, J = 8.0 Hz, 1H), 7.81 (d, J = 2.0 Hz, 1H), 7.65 (dd, J = 2.0, 8.4 Hz, 1H), 7.61-7.56 (m, 1H), 7.55-7.48 (m, 1H), 3.90 (s, 3H). |

General method for the synthesis of Example 135-137:

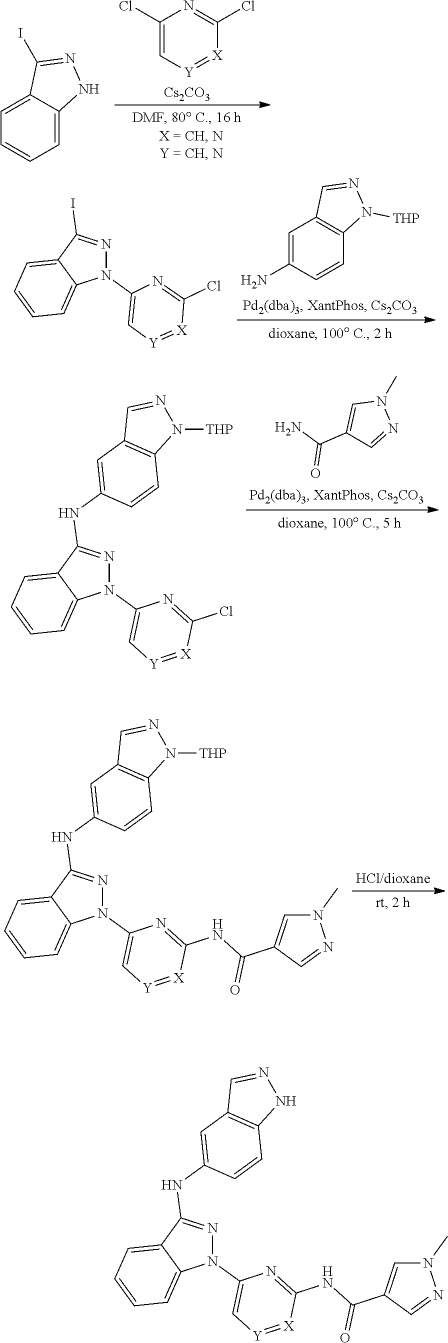

### Step 1: 1-(2-chloropyrimidin-4-yl)-3-iodo-indazole

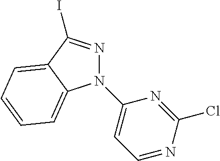 [SMILES: Clc1nccc(-n2nc(I)c3ccccc32)n1]

To a solution of 3-iodo-1H-indazole (200 mg, 819.57 umol, 1 eq) and 2,4-dichloropyrimidine (244.19 mg, 1.64 mmol, 2 eq) in DMF (10 mL) was added Cs₂CO₃ (534.06 mg, 1.64 mmol, 2 eq). The mixture was stirred at 60° C. for 1 hr. The reaction mixture was filtered and concentrated under reduced pressure to give a residue. The residue was purified by flash silica gel chromatography eluting with 0-30% EtOAc in Pet. Ether to afford 1-(2-chloropyrimidin-4-yl)-3-iodo-indazole (1.5 g, 4.21 mmol, 51.37% yield) as a white solid. LC-MS (ES⁺, Method A), 0.481 min, m/z 356.9 [M+H]⁺. tH NMR (400 MHz, CHLOROFORM-d) 6=8.75 (d, J=8.4 Hz, 1H), 8.58 (d, J=5.6 Hz, 1H), 7.94-7.88 (m, 1H), 7.71-7.65 (m, 1H), 7.57-7.51 (m, 1H), 7.47-7.41 (m, 1H).

### Step 2: 1-(2-chloropyrimidin-4-yl)-N-(1-tetrahydropyran-2-ylindazol-5-yl)indazol-3-amine

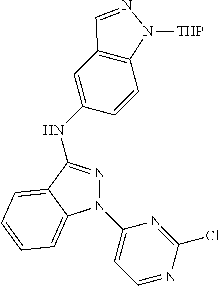

A mixture of 1-(2-chloropyrimidin-4-yl)-3-iodo-indazole (100 mg, 280.47 umol, 1 eq), 1-tetrahydropyran-2-ylindazol-5-amine (67.03 mg, 308.51 umol, 1.1 eq), Pd₂(dba)₃ (17.98 mg, 19.63 umol, 0.07 eq), Xantphos (22.72 mg, 39.27 umol, 0.14 eq) and Cs₂CO₃ (91.38 mg, 280.47 umol, 1 eq) in dioxane (1 mL) was degassed and purged with N₂ for 3 times, and then the mixture was stirred at 100° C. for 1 hr under N₂ atmosphere. The reaction mixture was filtered and concentrated under reduced pressure to give a residue. The residue was purified by flash silica gel chromatography eluting with 0-100% EtOAc in Pet. Ether to afford 1-(2-chloropyrimidin-4-yl)-N-(1-tetrahydropyran-2-ylindazol-5-yl)indazol-3-amine (102 mg, 228.75 umol, 81.56% yield) as a white solid. LC-MS (ES⁺, Method A), 0.561 min, m/z 446.1 [M+H]⁺.

### Step 3:1-methyl-N-[4-[3-[(1-tetrahydropyran-2-ylindazol-5-yl)amino]indazol-1-yl]pyrimidin-2-yl]pyrazole-4-carboxamide

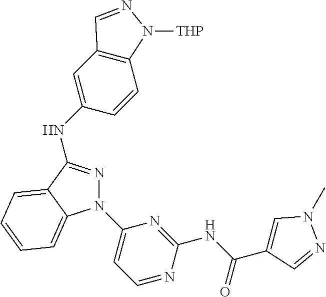

A mixture of 1-(2-chloropyrimidin-4-yl)-N-(1-tetrahydropyran-2-ylindazol-5-yl)indazol-3-amine (50 mg, 112.13 umol, 1 eq), 1-methylpyrazole-4-carboxamide (23.85 mg, 190.62 umol, 1.7 eq), Pd₂(dba)₃ (5.13 mg, 5.61 umol, 0.05 eq), Xantphos (6.49 mg, 11.21 umol, 0.1 eq) and Cs₂CO₃ (36.53 mg, 112.13 umol, 1 eq) in dioxane (2 mL) was degassed and purged with N₂ for 3 times, and then the mixture was stirred at 100° C. for 1 hr under N₂ atmosphere. The reaction mixture filtered and concentrated under reduced pressure to give a residue. The residue was purified by flash silica gel chromatography eluting with 0-100% EtOAc in Pet. Ether to afford 1-methyl-N-[4-[3-[(1-tetrahydropyran-2-ylindazol-5-yl)amino]indazol-1-yl]pyrimidin-2-yl]pyrazole-4-carboxamide (50 mg, 93.53 umol, 83.41% yield) as a white solid. LC-MS (ES⁺, Method A), 0.424 min, m/z 535.2 [M+H]⁺.

### Example 135: N-[4-[3-(1H-indazol-5-ylamino)indazo]-1-yl]pyrimidin-2-yl]-1-methyl-pyrazole-4-carboxamide

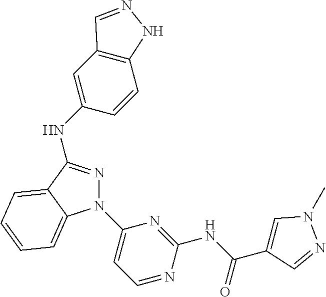 [SMILES: Cn1cc(C(=O)Nc2nccc(-n3nc(Nc4ccc5[nH]ncc5c4)c4ccccc43)n2)cn1]

To a solution of 1-methyl-N-[4-[3-[(1-tetrahydropyran-2-ylindazol-5-yl)amino]indazol-1-yl]pyrimidin-2-yl]pyrazole-4-carboxamide (50 mg, 93.53 umol, 1 eq) in HCl/dioxane (4 M). The mixture was stirred at 25° C. for 1 hr. The mixture was filtered, and then was concentrated under reduced pressure to give a residue. The crude product was triturated by MeOH (5 mL) at 25° C. for 30 min. The mixture was filtered, the filter cake was dried under vacuum to afford N-[4-[3-(1H-indazol-5-ylamino)indazo]-1-yl]pyrimidin-2-yl]-1-methyl-pyrazole-4-carboxamide (32.3 mg, 59.77 umol, 63.90% yield, 90.1% purity, HCl) as a yellow solid. LC-MS (ES⁺, Method A), 0.368 min, m/z 451.3 [M+H]⁺. ¹H NMR (400 MHz, DMSO-d6) 6=11.39 (s, 1H), 9.69 (s, 1H), 9.28 (s, 1H), 8.65-8.48 (m, 3H), 8.38-8.31 (m, 1H), 8.26-8.22 (m, 1H), 8.09 (s, 1H), 7.76-7.65 (m, 3H), 7.61-7.55 (m, 1H), 7.53-7.44 (m, 1H), 3.94 (s, 3H).

Compounds prepared in a similar manner to that set out above are given below in Table 27.

| Example | Structure | LCMS | ¹H NMR |
|---|---|---|---|
| 136 | 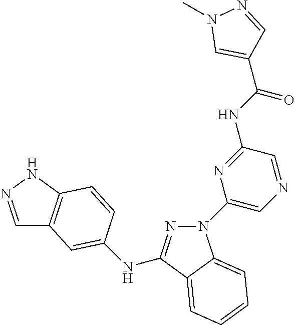 [SMILES: Cn1cc(C(=O)Nc2cncc(-n3nc(Nc4ccc5[nH]ncc5c4)c4ccccc43)n2)cn1] | LC-MS (ES⁺, Method A), 0.436 min, m/z 451.1 [M + H]⁺. | ¹H NMR (400 MHz, DMSO-d6) δ = 10.62 (s, 1H), 9.48 (s, 1H), 9.11 (s, 1H), 9.08-9.03 (m, 1H), 9.00 (s, 1H), 8.54 (s, 2H), 8.29 (d, J = 8.0 Hz, 1H), 8.16 (d, J = 17.6 Hz, 2H), 7.73-7.62 (m, 2H), 7.57 (d, J = 8.8 Hz, 1H), 7.39 (t, J = 7.6 Hz, 1H), 3.95 (s, 3H). |
| 137 | 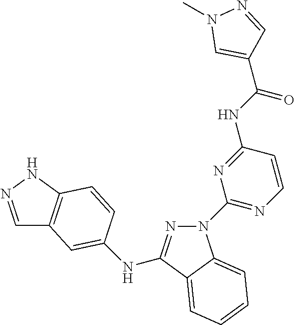 [SMILES: Cn1cc(C(=O)Nc2ccnc(-n3nc(Nc4ccc5[nH]ncc5c4)c4ccccc43)n2)cn1] | LC-MS (ES⁺, Method A), 0.379 min, m/z 451.2 [M + H]⁺. | ¹H NMR (400 MHz, DMSO-d6) δ = 13.32 (s, 1H), 11.11 (s, 1H), 9.50 (s, 1H), 9.02-8.97 (m, 1H), 8.72 (d, J = 6.0 Hz, 1H), 8.67 (d, J = 1.6 Hz, 1H), 8.63-8.57 (m, 1H), 8.33-8.27 (m, 1H), 8.22 (s, 1H), 8.07 (s, 1H), 7.96-7.91 (m, 1H), 7.76-7.64 (m, 2H), 7.58-7.53 (m, 1H), 7.46- 7.39 (m, 1H), 3.95 (s, 3H). |

### General Route or the Synthesis of Examples 138-141:

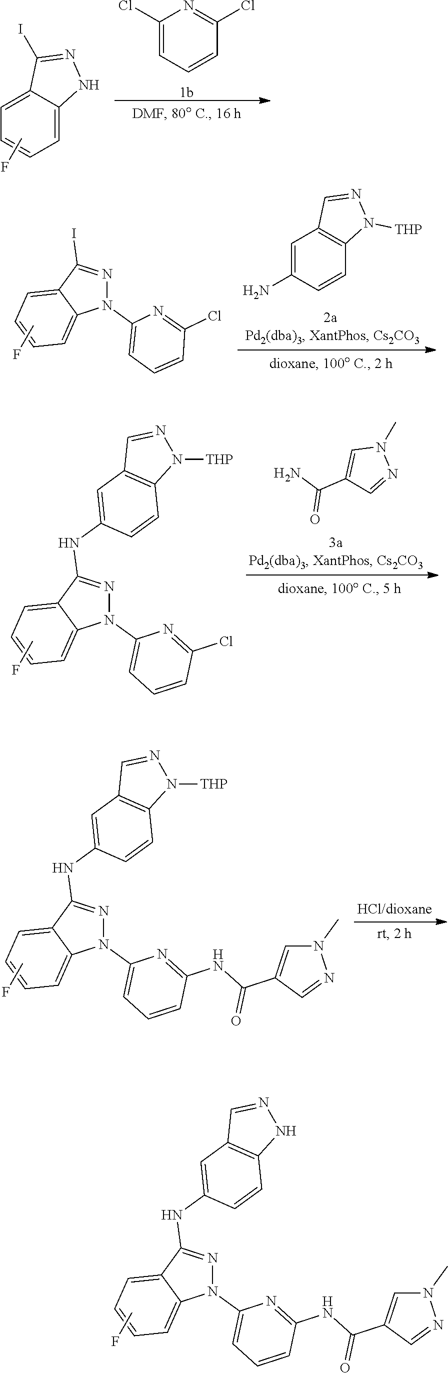

### Step 1: 1-(6-chloro-2-pyridyl)-4-fluoro-3-iodo-indazole

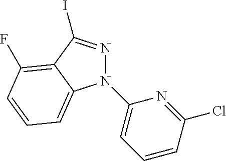 [SMILES: Fc1cccc2c1c(I)nn2-c1cccc(Cl)n1]

To a solution of 2,6-dichloropyridine (423.60 mg, 2.86 mmol, 1.5 eq) in DMF (5 mL) was added Cs₂CO₃ (1.24 g, 3.82 mmol, 2 eq) and 4-fluoro-3-iodo-1H-indazole (500.00 mg, 1.91 mmol, 1 eq). The mixture was stirred at 80° C. for 16 hr. The reaction mixture was pour into H₂O 50 mL then stirred for 15 min, the mixture was filtered and the filter cake was concentrated in vacuo. The residue was purified by column chromatography eluting with 0-10% EtOAc in Pet. Ether to afford 1-(6-chloro-2-pyridyl)-4-fluoro-3-iodo-indazole (300 mg, 803.10 umol, 42.09% yield) as a yellow solid. LC-MS (ES⁺, Method A), 0.60 min, m/z 373.1 [M+H]⁺*.

### Step 2: 1-(6-chloro-2-pyridyl)-4-fluoro-N-(1-tetrahydropyran-2-ylindazol-5-yl)indazol-3-amine

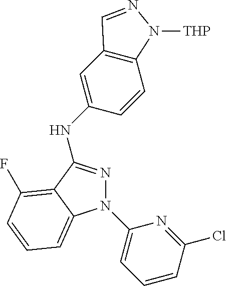

A mixture of 1-(6-chloro-2-pyridyl)-4-fluoro-3-iodo-indazole (264.78 mg, 708.81 umol, 1.1 eq), 1-tetrahydropyran-2-ylindazol-5-amine (140 mg, 644.37 umol, 1 eq), Pd₂(dba)₃ (59.01 mg, 64.44 umol, 0.1 eq), Xantphos (74.57 mg, 128.87 umol, 0.2 eq) and Cs₂CO₃ (419.90 mg, 1.29 mmol, 2 eq) in dioxane (3 mL) was degassed and purged with N₂ for 3 times, and then the mixture was stirred at 100° C. for 16 hr under N₂ atmosphere. The reaction mixture was filtered and concentrated under reduced pressure to give a residue. The residue was purified by preparative-TLC (SiO₂, Petroleum ether: Ethyl acetate=3/1) to afford 1-(6-chloro-2-pyridyl)-4-fluoro-N-(1-tetrahydropyran-2-ylindazol-5-yl)indazol-3-amine (50 mg, 108.01 umol, 16.76% yield) as a white solid. LC-MS (ES⁺, Method A), 0.60 min, m/z 463.1 [M+H]⁺*.

### Step 3: N-[6-[4-fluoro-3-[(1-tetrahydropyran-2-ylindazol-5-yl)amino]indazol-1-yl]-2-pyridyl]-1-methyl-pyrazole-4-carboxamide

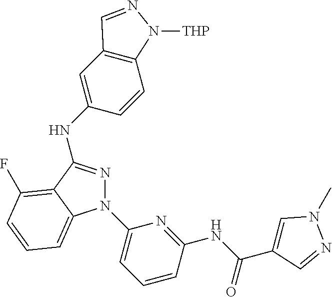

A mixture of 1-(6-chloro-2-pyridyl)-4-fluoro-N-(1-tetrahydropyran-2-ylindazol-5-yl)indazol-3-amine (30 mg, 64.81 umol, 1 eq), 1-methylpyrazole-4-carboxamide (8.92 mg, 71.29 umol, 1.1 eq), Pd₂(dba)₃ (5.93 mg, 6.48 umol, 0.1 eq), Xantphos (7.50 mg, 12.96 umol, 0.2 eq) and Cs₂CO₃ (42.23 mg, 129.62 umol, 2 eq) in dioxane (2 mL) was degassed and purged with N₂ for 3 times, and then the mixture was stirred at 100° C. for 16 hr under N₂ atmosphere. The reaction mixture was filtered and concentrated under reduced pressure to give a residue. The residue was purified by preparative-TLC (SiO₂, Petroleum ether: Ethyl acetate=5/1) to afford N-[6-[4-fluoro-3-[(1-tetrahydropyran-2-ylindazol-5-yl)amino]indazol-1-yl]-2-pyridyl]-1-methyl-pyrazole-4-carboxamide (16 mg, 29.01 umol, 44.76% yield) as a yellow solid. LC-MS (ES⁺, Method A), 0.53 min, m/z 552.4 [M+H]⁺*.

### Example 138: N-[6-[4-fluoro-3-(1H-indazol-5-ylamino)indazol-1-yl]-2-pyridyl]-1-methyl-pyrazole-4-carboxamide

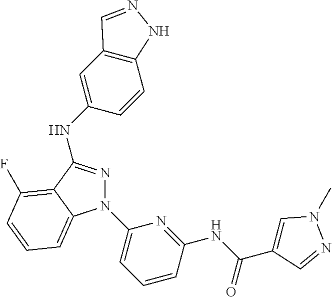 [SMILES: Cn1cc(C(=O)Nc2cccc(-n3nc(Nc4ccc5[nH]ncc5c4)c4c(F)cccc43)n2)cn1]

To a solution of N-[6-[4-fluoro-3-[(1-tetrahydropyran-2-ylindazol-5-yl)amino]indazol-1-yl]-2-pyridyl]-1-methyl-pyrazole-4-carboxamide (16 mg, 29.01 umol, 1 eq) in HCl/dioxane (2 mL) was stirred at 25° C. for 1 hr. The reaction mixture was filtered and concentrated under reduced pressure to give a residue. The residue was purified by preparative HPLC (36-66% MeCN in H₂O) to afford N-[6-[4-fluoro-3-(1H-indazol-5-ylamino)indazol-1-yl]-2-pyridyl]-1-methyl-pyrazole-4-carboxamide (5.1 mg, 10.07 umol, 34.72% yield, 92.311% purity) as a white solid. LC-MS (ES⁺, Method A), 0.48 min, m/z 468.1 [M+H]⁺. ¹H NMR (400 MHz, DMSO-d6) δ 12.92 (s, 1H), 10.35 (s, 1H), 8.99 (d, J=8.4 Hz, 1H), 8.58-8.46 (m, 2H), 8.37 (s, 1H), 8.15 (s, 1H), 8.07 (s, 1H), 8.01-7.89 (m, 2H), 7.72 (dd, J=1.6, 8.8 Hz, 1H), 7.65 (d, J=7.6 Hz, 1H), 7.59 (dt, J=5.6, 8.4 Hz, 1H), 7.53 (d, J=9.2 Hz, 1H), 7.09 (dd, J=8.0, 10.4 Hz, 1H), 3.94 (s, 3H).

Compounds prepared in a similar manner to that set out above are given below in Table 28.

| Example | Structure | LCMS | ¹H NMR |
|---|---|---|---|
| 139 | 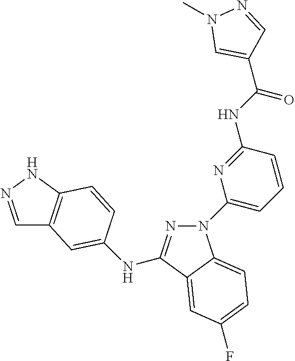 [SMILES: Cn1cc(C(=O)Nc2cccc(-n3nc(Nc4ccc5[nH]ncc5c4)c4cc(F)ccc43)n2)cn1] | LC-MS (ES⁺, Method A), 0.44 min, m/z 468.3 [M + H]⁺. | ¹H NMR (400 MHz, DMSO-d6) δ 10.34 (s, 1H), 9.39 (s, 1H), 9.20 (dd, J = 4.4, 9.2 Hz, 1H), 8.56-8.43 (m, 2H), 8.15 (s, 1H), 8.13-8.07 (m, 2H), 8.01-7.94 (m, 1H), 7.88 (d, J = 8.0 Hz, 1H), 7.72-7.64 (m, 2H), 7.56 (d, J = 8.8 Hz, 1H), 7.47 (dt, J = 2.8, 9.2 Hz, 1H), 3.94 (s, 3H). |
| 140 | 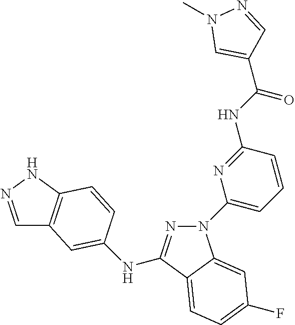 [SMILES: Cn1cc(C(=O)Nc2cccc(-n3nc(Nc4ccc5[nH]ncc5c4)c4ccc(F)cc43)n2)cn1] | LC-MS (ES⁺, Method A), 0.46 min, m/z 468.2 [M + H]⁺. | ¹H NMR (400 MHz, DMSO-d6) δ = 10.44 (s, 1H), 9.37 (d, J = 4.0 Hz, 1H), 9.08-8.92 (m, 1H), 8.49 (d, J = 7.2 Hz, 2H), 8.24 (dt, J = 3.2, 5.2 Hz, 1H), 8.14 (s, 1H), 8.09 (s, 1H), 8.03-7.95 (m, 1H), 7.93-7.87 (m, 1H), 7.74- 7.62 (m, 2H), 7.56 (d, J = 8.0 Hz, 1H), 7.25-7.17 (m, 1H), 3.95 (s, 3H). |
| 141 | 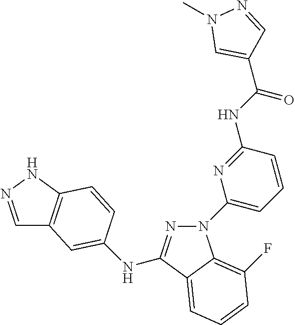 [SMILES: Cn1cc(C(=O)Nc2cccc(-n3nc(Nc4ccc5[nH]ncc5c4)c4cccc(F)c43)n2)cn1] | LC-MS (ES⁺, Method A), 0.44 min, m/z 468.1 [M + H]⁺. | ¹H NMR (400 MHz, DMSO-d6) δ = 10.39 (s, 1H), 9.28 (s, 1H), 8.48 (s, 1H), 8.40 (s, 1H), 8.19-7.97 (m, 5H), 7.64 (d, J = 8.0 Hz, 1H), 7.52 (dd, J = 5.2, 8.0 Hz, 2H), 7.38 (dd, J = 8.0, 12.0 Hz, 1H), 7.26 (td, J = 4.0, 8.0 Hz, 1H), 3.89 (s, 3H). |

### General Route or the Synthesis of Examples 142-145:

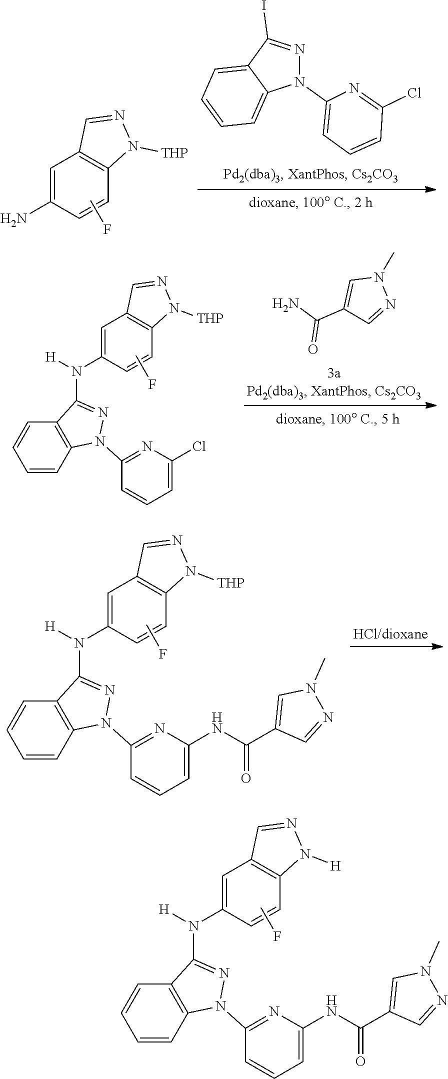

### Step 1: N-[1-(6-chloro-2-pyridyl)indazo]-3-yl]-4-fluoro-1-tetrahydropyran-2-yl-indazol-5-amine

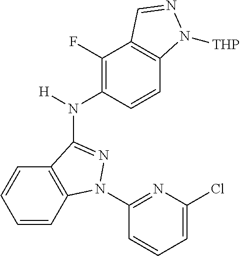

A mixture of 4-fluoro-1-tetrahydropyran-2-yl-indazol-5-amine (200 mg, 850.13 umol, 1 eq), 1-(6-chloro-2-pyridyl)-3-iodo-indazole (302.27 mg, 850.13 umol, 1 eq), Pd₂(dba)₃ (38.92 mg, 42.51 umol, 0.05 eq), Xantphos (49.19 mg, 85.01 umol, 0.1 eq) and Cs₂CO₃ (276.99 mg, 850.13 umol, 1 eq) in dioxane (1 mL) was degassed and purged with N₂ for 3 times, and then the mixture was stirred at 100° C. for 1 hr under N₂ atmosphere. The mixture was concentrated under reduced pressure. The residue was purified by flash silica gel chromatography eluting with 0-100% EtOAc in Pet. Ether to afford N-[1-(6-chloro-2-pyridyl)indazo]-3-yl]-4-fluoro-1-tetrahydropyran-2-yl-indazol-5-amine (300 mg, 648.08 umol, 76.23% yield) as a white solid. LC-MS (ES⁺, Method A), 0.580 min, m/z 463.1 [M+H]⁺.

### Step 2: N-[6-[3-[(4-fluoro-1-tetrahydropyran-2-yl-indazol-5-yl)amino]indazol-1-yl]-2-pyridyl]-1-methyl-pyrazole-4-carboxamide

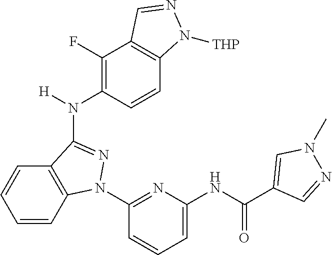

A mixture of N-[1-(6-chloro-2-pyridyl)indazo]-3-yl]-4-fluoro-1-tetrahydropyran-2-yl-indazol-5-amine (150 mg, 324.04 umol, 1 eq), 1-methylpyrazole-4-carboxamide (121.64 mg, 972.12 umol, 3 eq), Pd₂(dba)₃ (14.84 mg, 16.20 umol, 0.05 eq), Xantphos (18.75 mg, 32.40 umol, 0.1 eq) and Cs₂CO₃ (105.58 mg, 324.04 umol, 1 eq) in dioxane (10 mL) was degassed and purged with N₂ for 3 times, and then the mixture was stirred at 100° C. for 1 hr under N₂ atmosphere. The mixture was filtered, and then was concentrated under reduced pressure to give a residue. The residue was purified by preparative HPLC (32-62% MeCN in H₂O) to afford N-[6-[3-[(4-fluoro-1-tetrahydropyran-2-yl-indazol-5-yl)amino]indazol-1-yl]-2-pyridyl]-1-methyl-pyrazole-4-carboxamide (102 mg, 184.93 umol, 57.07% yield) as a white solid. LC-MS (ES⁺, Method A), 0.516 min, m/z 552.2 [M+H]⁺.

### Example 142: N-[6-[3-[(4-fluoro-1H-indazol-5-yl)amino]indazol-1-yl]-2-pyridyl]-1-methyl-pyrazole-4-carboxamide

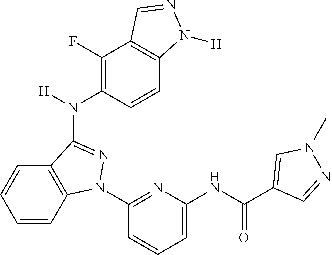 [SMILES: Cn1cc(C(=O)Nc2cccc(-n3nc(Nc4ccc5[nH]ncc5c4F)c4ccccc43)n2)cn1]

A mixture of N-[6-[3-[(4-fluoro-1-tetrahydropyran-2-yl-indazol-5-yl)amino]indazol-1-yl]-2-pyridyl]-1-methyl-pyrazole-4-carboxamide (50 mg, 90.65 umol, 1 eq) in HCl/dioxane (4 M) was stirred at 25° C. for 5 min. The mixture was filtered, and then was concentrated under reduced pressure to give a residue. The crude product was triturated with DCM:MeOH=20:1 at 25° C. for 30 min, and filtered, the filter cake was dried to afford N-[6-[3-[(4-fluoro-1H-indazol-5-yl)amino]indazol-1-yl]-2-pyridyl]-1-methyl-pyrazole-4-carboxamide (14.6 mg, 27.52 umol, 30.36% yield, 95% purity, HCl) as a green solid. LC-MS (ES⁺, Method A), 0.47 min, m/z 468.1 [M+H]⁺. ¹H NMR (400 MHz, DMSO-d6) δ=10.28 (s, 1H), 9.09 (d, J=8.4 Hz, 1H), 8.80 (s, 1H), 8.49 (s, 1H), 8.19 (s, 1H), 8.13 (s, 1H), 8.09 (d, J=8.0 Hz, 1H), 8.03-7.95 (m, 1H), 7.82 (d, J=4.0 Hz, 2H), 7.57 (t, J=7.6 Hz, 1H), 7.44 (d, J=8.8 Hz, 1H), 7.41-7.36 (m, 1H), 7.28 (t, J=7.6 Hz, 1H), 3.93 (s, 3H).

Compounds prepared in a similar manner to that set out above are given below in Table 29.

| Example | Structure | LCMS | ¹H NMR |
|---|---|---|---|
| 143 | 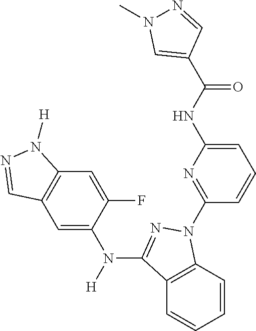 [SMILES: Cn1cc(C(=O)Nc2cccc(-n3nc(Nc4cc5cn[nH]c5cc4F)c4ccccc43)n2)cn1] | LC-MS (ES⁺, Method A), 0.47 min, m/z 468.0 [M + H]⁺. | ¹H NMR (400 MHz, DMSO-d6) δ = 10.33 (s, 1H), 9.17- 9.09 (m, 1H), 8.85 (s, 1H), 8.66-8.58 (m, 1H), 8.54 (s, 1H), 8.24-8.19 (m, 1H), 8.14 (s, 1H), 7.94-7.83 (m, 2H), 7.58 (d, J = 7.2 Hz, 1H), 7.52-7.44 (m, 1H), 7.34-7.21 (m, 2H), 7.11 (sDzhgcd33., 1H), 6.99 (s, 1H), 3.98 (s, 3H). |
| 144 | 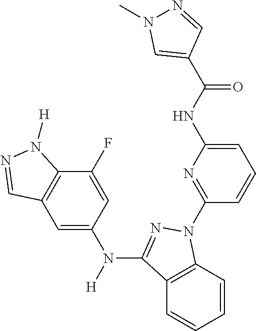 [SMILES: Cn1cc(C(=O)Nc2cccc(-n3nc(Nc4cc(F)c5[nH]ncc5c4)c4ccccc43)n2)cn1] | LC-MS (ES⁺, Method A), 0.47 min, m/z 468.5 [M + H]⁺. | ¹H NMR (400 MHz, DMSO-d6) δ = 10.33 (s, 1H), 9.30 (s, 1H), 9.17 (d, J = 8.4 Hz, 1H), 8.67 (d, J = 2.8 Hz, 1H), 8.51 (s, 1H), 8.33 (d, J = 8.0 Hz, 1H), 8.15 (s, 1H), 7.97- 7.87 (m, 3H), 7.66-7.59 (m, 2H), 7.36 (t, J = 7.2 Hz, 1H), 7.25-7.18 (m, 1H), 3.94 (s, 3H). |
| 145 | 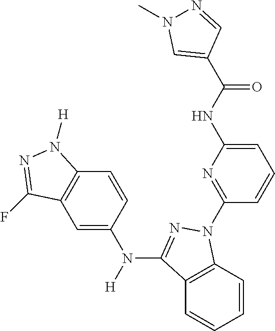 [SMILES: Cn1cc(C(=O)Nc2cccc(-n3nc(Nc4ccc5[nH]nc(F)c5c4)c4ccccc43)n2)cn1] | LC-MS (ES⁺, Method A), 0.46 min, m/z 468.2 [M + H]⁺. | ¹H NMR (400 MHz, DMSO-d6) δ = 12.33 (s, 1H), 10.36 (s, 1H), 9.47 (s, 1H), 9.15 (d, J = 8.4 Hz, 1H), 8.51 (s, 1H), 8.44-8.39 (m, 1H), 8.24-8.17 (m, 1H), 8.17 (s, 1H), 8.02-7.96 (m, 1H), 7.88 (d, J = 8.0 Hz, 1H), 7.77-7.71 (m, 1H), 7.64-7.57 (m, 2H), 7.54-7.48 (m, 1H), 7.38- 7.31 (m, 1H), 3.94 (s, 3H). |

General method for the synthesis of Example 146:

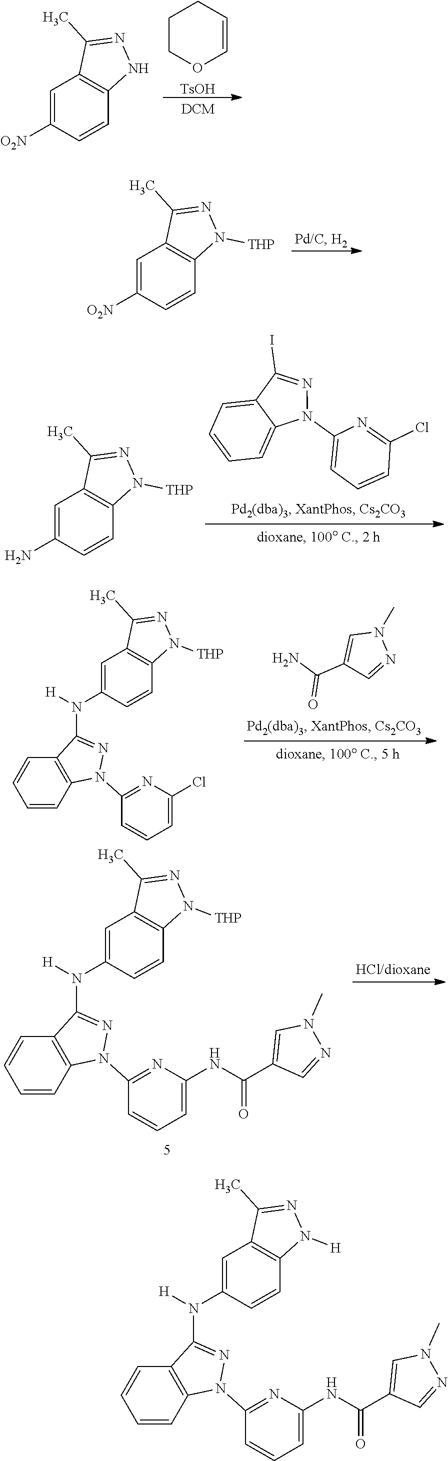

### Step 1: 3-methyl-5-nitro-1-tetrahydropyran-2-yl-indazole

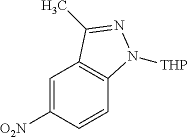

To a solution of 3-methyl-5-nitro-1H-indazole (1 g, 5.64 mmol, 1 eq) and 3,4-dihydro-2H-pyran (1.42 g 16.93 mmol, 1.55 mL, 3 eq) in DCM (20 mL) was added TsOH·H₂O (107.37 mg, 564.46 umol, 0.1 eq). The mixture was stirred at 25° C. for 16 hr. The mixture was concentrated under reduced pressure to afford 3-methyl-5-nitro-1-tetrahydropyran-2-yl-indazole (1.3 g, 4.98 mmol, 88.15% yield) as a white solid. LC-MS (ES⁺, Method A), 0.475 min, m/z 261.1 [M+H]⁺.

### Step 2: 3-methyl-1-tetrahydropyran-2-yl-indazol-5-amine

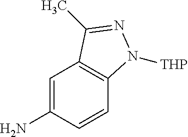

A mixture of 3-methyl-5-nitro-1-tetrahydropyran-2-yl-indazole (1.3 g, 4.98 mmol, 1eq), Pd/C (0.13 g, 10% purity) in MeOH (10 mL) was degassed and purged with N₂ for 3 times, and then the mixture was stirred at 25° C. for 16 hr under H₂ atmosphere. The reaction mixture was filtered and concentrated under reduced pressure to give 3-methyl-1-tetrahydropyran-2-yl-indazol-5-amine (1.1 g, 4.76 mmol, 95.58% yield) as a white solid. LC-MS (ES⁺, Method A), 0.210 min, m/z 232.1 [M+H]⁺.

### Step 3: N-[1-(6-chloro-2-pyridyl)indazo]-3-yl]-3-methyl-1-tetrahydropyran-2-yl-indazol-5-amine

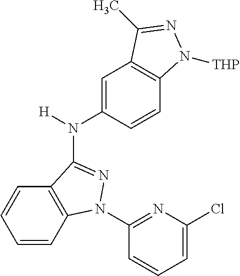

A mixture of 3-methyl-1-tetrahydropyran-2-yl-indazol-5-amine (200 mg, 864.70 umol, 1 eq), 1-(6-chloro-2-pyridyl)-3-iodo-indazole (307.46 mg, 864.70 umol, 1 eq), Pd₂(dba)₃ (39.59 mg, 43.24 umol, 0.05 eq), Xantphos (50.03 mg, 86.47 umol, 0.1 eq) and Cs₂CO₃ (281.74 mg, 864.70 umol, 1 eq) in dioxane (10 mL) was degassed and purged with N₂ for 3 times, and then the mixture was stirred at 100° C. for 16 hr under N₂ atmosphere. The reaction mixture was filtered and concentrated under reduced pressure to give a residue. The residue was purified by flash sili-a gel chromatography eluting with 0-100% EtOAc in Pet. Ether to afford N-[1-(6-chloro-2-pyridyl)indazo]-3-yl]-3-methyl-1-tetrahydropyran-2-yl-indazol-5-amine (350 mg, 762.62 umol, 88.19% yield) as a white solid. LC-MS (ES⁺, Method A), 0.667 min, m/z 459.2 [M+H]⁺.

### Step 4: 1-methyl-N-[6-[3-[(3-methyl-1-tetrahydropyran-2-yl-indazol-5-yl)amino]indazol-1-yl]-2-pyridyl]pyrazole-4-carboxamide

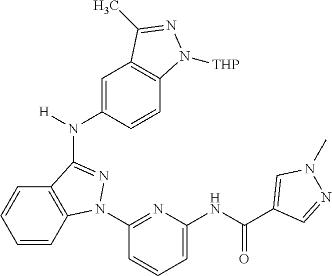

A mixture of N-[1-(6-chloro-2-pyridyl)indazo]-3-yl]-3-methyl-1-tetrahydropyran-2-yl-indazol-5-amine (50 mg, 108.95 umol, 1 eq), 1-methylpyrazole-4-carboxamide (17.72 mg, 141.63 umol, 1.3 eq), Pd₂(dba)₃ (4.99 mg, 5.45 umol, 0.05 eq), Xantphos (6.30 mg, 10.89 umol, 0.1 eq) and Cs₂CO₃ (35.50 mg, 108.95 umol, 1 eq) in dioxane (1 mL) was degassed and purged with N₂ for 3 times, and then the mixture was stirred at 100° C. for 1 hr under N₂ atmosphere. The mixture was filtered, and then was concentrated under reduced pressure to give a residue. The residue was triturated with MeOH at 25° C. for 30 min. The mixture was filtered, the filter cake was dried under vacuum to afford 1-methyl-N-[6-[3-[(3-methyl-1-tetrahydropyran-2-yl-indazol-5-yl)amino]indazol-1-yl]-2-pyridyl]pyrazole-4-carboxamide (59 mg, 107.74 umol, 98.89% yield) as a white solid. LC-MS (ES⁺, Method A), 0.591 min, m/z 548.2 [M+H]⁺.

### Example 146: 1-methyl-N-[6-[3-[(3-methyl-1H-indazol-5-yl)amino]indazol-1-yl]-2-pyridyl]pyrazole-4-carboxamide

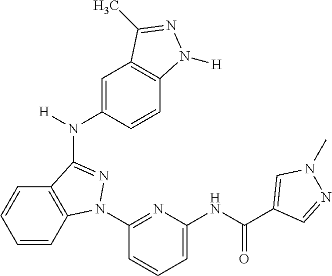 [SMILES: Cc1n[nH]c2ccc(Nc3nn(-c4cccc(NC(=O)c5cnn(C)c5)n4)c4ccccc34)cc12]

A mixture of 1-methyl-N-[6-[3-[(3-methyl-1-tetrahydropyran-2-yl-indazol-5-yl)amino]indazol-1-yl]-2-pyridyl]pyrazole-4-carboxamide (100 mg, 182.61 umol, 1 eq) in HCl/dioxane (4 M) was stirred at 25° C. for 2 hr. The mixture was filtered, and then was concentrated under reduced pressure to give a residue. The residue was triturated with MeOH at 25° C. for 30 min. The mixture was filtered, the filter cake was dried under vacuum to afford 1-methyl-N-[6-[3-[(3-methyl-1H-indazol-5-yl)amino]indazol-1-yl]-2-pyridyl]pyrazole-4-carboxamide (35.8 mg, 66.09 umol, 36.19% yield, 92.3% purity, HCl) as a yellow solid. LC-MS (ES⁺, Method A), 0.46 min, m/z 464.2 [M+H]⁺. ¹H NMR (400 MHz, DMSO-d6) 6=10.31 (s, 1H), 9.44 (s, 1H), 9.15 (d, J=8.4 Hz, 1H), 8.52 (s, 1H), 8.43 (d, J=1.6 Hz, 1H), 8.22 (d, J=8.0 Hz, 1H), 8.15 (s, 1H), 8.01-7.93 (m, 1H), 7.90-7.85 (m, 1H), 7.75-7.69 (m, 1H), 7.67-7.57 (m, 2H), 7.49 (d, J=8.8 Hz, 1H), 7.33 (t, J=7.6 Hz, 1H), 3.94 (s, 3H), 2.56 (s, 3H).

### General Route or the Synthesis of Example 147:

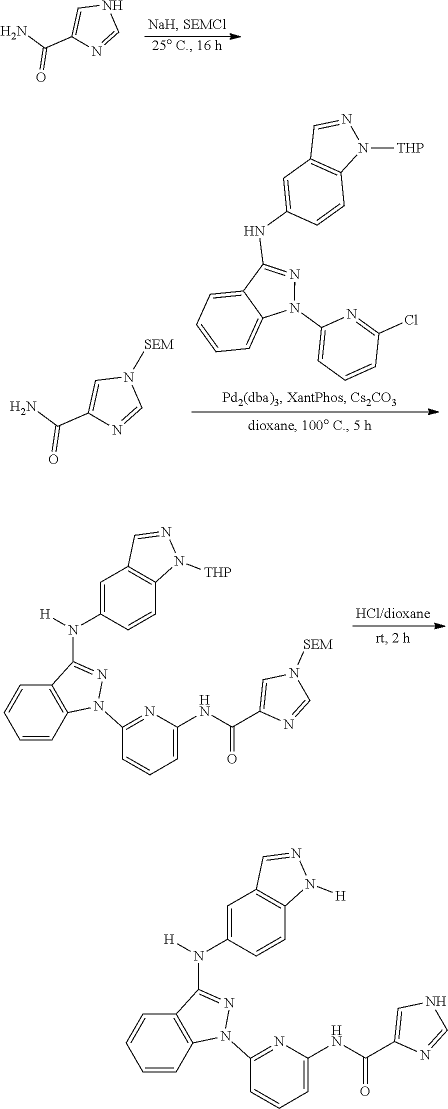

### Step 1: 3-((2-(trimethylsilyl)ethoxy)methyl)-3H-pyrrole-5-carboxamide

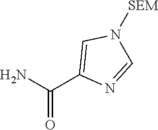 [SMILES: C[Si](C)(C)CCOCn1cnc(C(N)=O)c1]

To a mixture of 1H-imidazole-4-carboxamide (100 mg, 900.08 umol, 1 eq) and NaH (36.00 mg, 900.08 umol, 60% purity, 1 eq) in THF (2 mL) at 0° ′C and stirred for 1 hr, then SEM-C1 (150.06 mg, 900.08 umol, 159.30 uL, 1 eq) was added dropwise. The mixture was stirred at 25° C. for 15 hr. The reaction mixture was quenched by addition of H₂O (1 mL) at 0° C., and then diluted with H₂O (1 mL) and extracted with EtOAc (2 mL* 3). The combined organic layers were washed with NaCl aqueous solution (3 mL), dried over Na₂SO₄, filtered and concentrated under reduced pressure to give 1-(2-trimethylsilylethoxymethyl)imidazole-4-carboxamide (100 mg, 414.32 umol, 46.03% yield) was obtained as a yellow oil. LC-MS (ES⁺, Method A), 0.33 min, m/z 242.2 [M+H]⁺.

### Step 2: N-(6-(3-((1-(tetrahydro-2H-pyran-2-yl)-1H-indazol-5-yl)amino)-1H-indazol-1-yl)pyridin-2-yl)-1-((2-(trimethylsilyl)ethoxy)methyl)-1H-imidazole-4-carboxamide

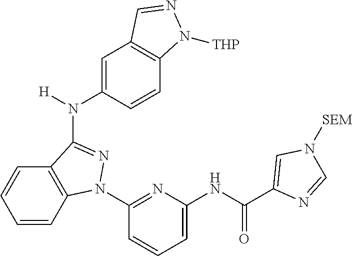

A mixture of 1-(6-chloro-2-pyridyl)-N-(1-tetrahydropyran-2-ylindazol-5-yl)indazol-3-amine (140 mg, 314.67 umol, 1 eq), 1-(2-trimethylsilylethoxymethyl)imidazole-4-carboxamide (91.14 mg 377.60 umol, 1.2 eq) Xantphos (36.41 mg, 62.93 umol, 0.2 eq), Pd₂(dba)₃ (28.81 mg, 31.47 umol, 0.1 eq) and Cs₂CO₃ (205.05 mg, 629.33 umol, 2 eq) in dioxane (1 mL) was degassed and purged with N₂ for 3 times, and then the mixture was stirred at 100° C. for 16 hr under N₂ atmosphere. The reaction mixture was concentrated under reduced pressure to give a residue. The residue was purified by flash silica gel chromatography eluting with 0-33% EtOAc in Pet. Ether to give N-[6-[3-[(1-tetrahydropyran-2-ylindazol-5-yl)amino]indazol-1-yl]-2-pyridyl]-1-(2-trimethylsilylethoxymethyl)imidazole-4-carboxamide (30 mg, 46.17 umol, 14.67% yield) as a yellow solid. LC-MS (ES⁺, Method A), 0.59 min, m/z 650.7 [M+H]⁺*.

### Example 147: N-(6-(3-((1H-indazol-5-yl)amino)-1H-indazol-1-yl)pyridin-2-yl)-1H-imidazole-4-carboxamide

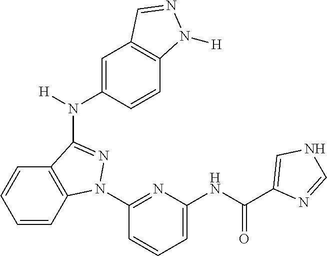 [SMILES: O=C(Nc1cccc(-n2nc(Nc3ccc4[nH]ncc4c3)c3ccccc32)n1)c1c[nH]cn1]

A mixture of N-[6-[3-[(1-tetrahydropyran-2-ylindazol-5-yl)amino]indazol-1-yl]-2-pyridyl]-1-(2-trimethylsilylethoxymethyl)imidazole-4-carboxamide (30 mg, 46.17 umol, 1 eq) in HCl/dioxane (1 mL) was stirred at 25° C. for 16 hr. The reaction mixture was concentrated under reduced pressure to give a residue. The residue was purified by preparative HPLC (18-48% MeCN in H₂O) to give N-[6-[3-(1H-indazol-5-ylamino)indazo]-1-yl]-2-pyridyl]-1H-imidazole-4-carboxamide (8.4 mg, 16.31 umol, 35.34% yield, 91.650% purity, HCl) as a yellow solid. LC-MS (ES⁺, Method A), 0.40 min, m/z 436.1 [M+H]⁺*. tH NMR (400 MHz, DMSO-d6) 6=11.02 (s, 1H), 9.46-9.37 (m, 1H), 9.21 (s, 1H), 9.13 (d, J=8.4 Hz, 1H), 8.67 (s, 1H), 8.52 (d, J=1.6 Hz, 1H), 8.27 (d, J=8.0 Hz, 1H), 8.08 (d, J=0.8 Hz, 1H), 8.05-8.00 (m, 1H), 7.84 (d, J=7.6 Hz, 1H), 7.77 (d, J=8.0 Hz, 1H), 7.70 (dd, J=2.0, 9.2 Hz, 1H), 7.62-7.51 (m, 2H), 7.33 (t, J=7.6 Hz, 1H).

### General Route or the Synthesis of Example 148:

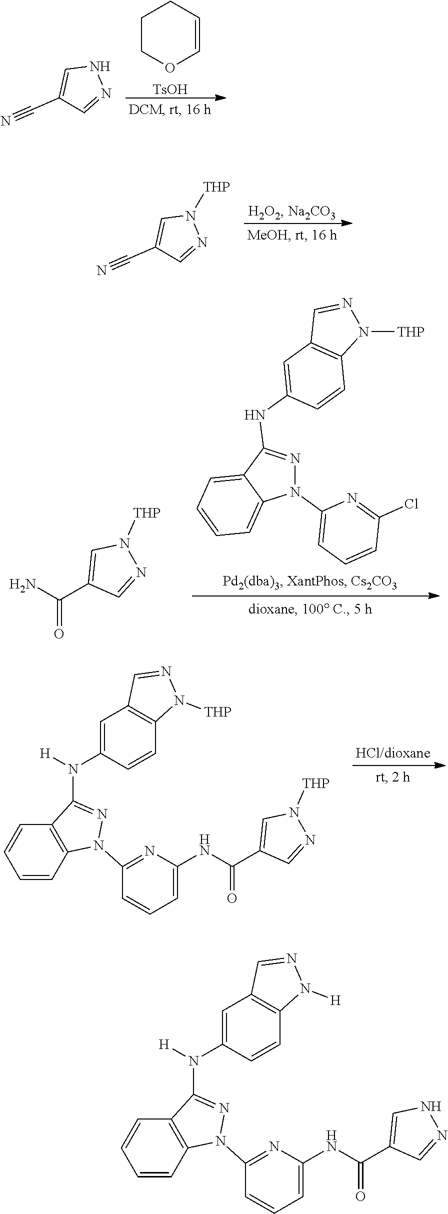

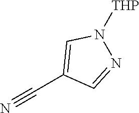

To a mixture of 1H-pyrazole-4-carbonitrile (1 g, 10.74 mmol, 1 eq) and TsOH·H₂O (204.34 mg, 1.07 mmol, 0.1 eq) in DCM (10 mL) was added 3,4-dihydro-2H-pyran (1.08 g, 12.89 mmol, 1.18 mL, 1.2 eq) dropwise at 0° C. over a period of 5 min. The reaction was then stirred at 25° C. for 13 hr. The mixture was then washed with 2 M aqueous Na₂CO₃ solution (20 mL) and water (20 mL), the organic layer was dried over Na₂SO₄, filtered and concentrated. The residue was triturated with EtOAc and the solid was collected and purified by column chromatography eluting with 0-30% EtOAc in Pet. Ether to give 1-tetrahydropyran-2-ylpyrazole-4-carbonitrile (560 mg, 3.16 mmol, 29.42% yield) as a white solid.

### Step 2: 1-(tetrahydro-2H-pyran-2-yl)-1H-pyrazole-4-carboxamide

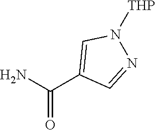

1-tetrahydropyran-2-ylpyrazole-4-carbonitrile (560 mg, 3.16 mmol, 1 eq) was dissolved in MeOH (5 mL) to which was added H₂O₂ (3.61 g, 35.02 mmol, 3.06 mL, 33% purity, 11.08 eq) followed by Na₂CO₃ (3 M, 3.16 mL, 3 eq). The reaction mixture was stirred at 25° C. for 4 hr. The reaction mixture was partitioned between EtOAc 20 mL and aqueous NaCl 10 ml. The organic phase was separated and the aqueous phase was extracted with ethyl acetate 20 ml*3 and the combined organics stirred with solid Na₂SO₃ followed by drying over Na₂SO₄, filtered and concentration under vacuum to give 1-tetrahydropyran-2-ylpyrazole-4-carboxamide (540 mg, 2.77 mmol, 87.53% yield) as a white solid.

### Step 3: 1-(tetrahydro-2H-pyran-2-yl)-N-(6-(3-((1-(tetrahydro-2H-pyran-2-yl)-1H-indazol-5-yl)amino)-1H-indazol-1-yl)pyridin-2-yl)-1H-pyrazole-4-carboxamide

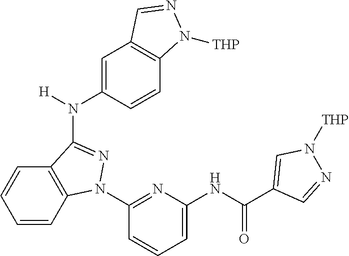

A mixture of 1-(6-chloro-2-pyridyl)-N-(1-tetrahydropyran-2-ylindazol-5-yl)indazol-3-amine (200 mg, 449.52 umol, 1 eq), 1-tetrahydropyran-2-ylpyrazole-4-carboxamide (175.51 mg, 899.05 umol, 2 eq), Pd₂(dba)₃ (41.16 mg, 44.95 umol, 0.1 eq), Xantphos (52.02 mg, 89.90 umol, 0.2 eq) and Cs₂CO₃ (439.39 mg, 1.35 mmol, 3 eq) in dioxane (5 mL) was degassed and purged with N₂ for 3 times, and then the mixture was stirred at 100° C. for 16 hr under N₂ atmosphere. The reaction mixture was concentrated to give a residue. The residue was purified by eluting with 0-80% EtOAc in Pet. Ether to give 1-tetrahydropyran-2-yl-N-[6-[3-[(1-tetrahydropyran-2-ylindazol-5-yl)amino]indazol-1-yl]-2-pyridyl]pyrazole-4-carboxamide (180 mg, 298.18 umol, 66.33% yield) as a yellow oil. LC-MS (ES⁺, Method A), 0.52 min, m/z 604.6 [M+H]⁺*.

### Example 148: N-(6-(3-((1H-indazol-5-yl)amino)-1H-indazol-1-yl)pyridin-2-yl)-1H-pyrazole-4-carboxamide

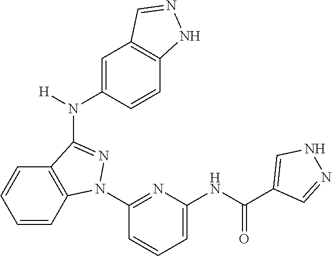 [SMILES: O=C(Nc1cccc(-n2nc(Nc3ccc4[nH]ncc4c3)c3ccccc32)n1)c1cn[nH]c1]

To a mixture of 1-tetrahydropyran-2-yl-N-[6-[3-[(1-tetrahydropyran-2-ylindazol-5-yl)amino]indazol-1-yl]-2-pyridyl]pyrazole-4-carboxamide (180 mg, 298.18 umol, 1 eq) in HCl/dioxane (10 mL). The mixture was stirred at 25° C. for 16 hr. The reaction mixture was concentrated under reduced pressure to give a residue. The crude product was triturated with MeOH at 25° C. for 2 h, filtered and the filter cake was concentrated in vacuum to give N-[6-[3-(1H-indazol-5-ylamino)indazo]-1-yl]-2-pyridyl]-1H-pyrazole-4-carboxamide (101.5 mg, 201.70 umol, 67.64% yield, 93.776% purity, HCl) as a yellow solid. LC-MS (ES⁺, Method A), 0.42 min, m/z 436.4 [M+H]⁺*. ¹H NMR (400 MHz, DMSO-d₆) δ=10.30 (s, 1H), 9.51-9.23 (m, 1H), 9.14 (d, J=8.4 Hz, 1H), 8.53 (d, J=1.6 Hz, 1H), 8.39 (s, 2H), 8.25 (d, J=8.0 Hz, 1H), 8.10 (s, 1H), 8.01-7.93 (m, 1H), 7.91-7.85 (m, 1H), 7.75-7.66 (m, 2H), 7.65-7.52 (m, 2H), 7.32 (t, J=7.6 Hz, 1H).

ROCK2 and ROCK] kinase assays

ROCK2 and ROCK1 enzyme potencies were determined by Reaction Biology (www.reactionbiology.com) using their Hot Spot kinase Assay. The base reaction buffer for the assay was 20 mM Hepes (pH 7.5), 10 mM MgCl₂, 1 mM EGTA, 0.01% Brij35, 0.02 mg/mL BSA, 0.1 mM Na₃VO₄, and 2 mM DTT with a 1% DMSO concentration. Required cofactors were added individually to each kinase reaction. The substrate was freshly prepared in the reaction buffer described above, and then cofactors were delivered. The purified kinase was added to the substrate solution and then gently mixed. Compounds were added from 100% DMSO into the kinase reaction mixture by Acoustic technology (Echo550; nanoliter range) and then incubated for 20 min at room temperature. ³³P-ATP (10 μM) was delivered to initiate the reaction, and then the mixture was incubated again for two hours at room temperature. Kinase activity was determined by P81 filter-binding method as described in the following reference: Anastassiadis T, et al. Comprehensive assay of kinase catalytic activity reveals features of kinase inhibitor selectivity. Nat. Biotechnol. 2011 Oct. 30; 29(11):1039-45. doi: 10.1038/nbt.2017.

Table 30 demonstrates ROCK2 and ROCK1 binding activity as determined by the assay described above for certain compounds. The compounds were categorized based on IC₅₀ value as “+”, “++”, “+++”, and “++++”. The category “+” refers to compounds with ROCK IC₅₀ value of >10 μM. The category “++” refers to compounds with a ROCK IC₅₀ value of 10 to 3 μM. The category “+++” refers to compounds with ROCK IC₅₀ value of 3 to 0.3 μM. The category “++++” refers to compounds with a ROCK IC₅₀ value of <0.3 μM. “ND” refers to “not determined.” The compound of an “Example Number” is the final product of an Example entitled the Example Number.

TABLE 30
ROCK2 and ROCK1 binding activity as determined by the
kinase assay for exemplary compounds.

| Example Number | ROCK2 IC₅₀ | ROCK1 IC₅₀ |
|---|---|---|
| 1 | +++ | ++ |
| 2 | ND | ND |
| 3 | ++++ | ++++ |
| 4 | ++++ | ++++ |
| 5 | ++++ | +++ |
| 6 | +++ | +++ |
| 7 | ND | ND |
| 8 | ND | ND |
| 9 | ++++ | +++ |
| 10 | ND | ND |
| 11 | +++ | + |
| 12 | ++++ | +++ |
| 13 | ++++ | +++ |
| 14 | +++ | + |
| 15 | ND | ND |
| 16 | +++ | + |
| 17 | ++++ | +++ |
| 18 | +++ | ++ |
| 19 | ++++ | ++++ |
| 20 | ND | ND |
| 21 | +++ | ++ |
| 22 | +++ | ++ |
| 23 | ND | ND |
| 24 | ND | ND |
| 25 | +++ | +++ |
| 26 | +++ | ++ |
| 27 | +++ | ++ |
| 28 | ND | ND |
| 29 | ND | ND |
| 30 | ND | ND |
| 31 | ++++ | +++ |
| 32 | ++++ | + |
| 33 | ++++ | ++++ |
| 34 | ++++ | ++++ |
| 35 | ++++ | +++ |
| 36 | ++++ | ++++ |
| 37 | + | + |
| 38 | ++++ | ++++ |
| 39 | ++++ | ++++ |
| 40 | ++++ | ++++ |
| 41 | ++++ | +++ |
| 42 | ++++ | ++++ |
| 43 | ND | ND |
| 44 | ++++ | ++++ |
| 45 | ++++ | ++++ |
| 46 | ++++ | ++++ |
| 47 | + | + |
| 48 | ++++ | ++++ |
| 49 | ++++ | ++++ |
| 50 | ++++ | ++++ |
| 51 | ND | ND |
| 52 | ++++ | ++++ |
| 53 | ++++ | ++ |
| 54 | + | + |
| 55 | ++++ | ++++ |
| 56 | ++++ | +++ |
| 57 | ++++ | ++++ |
| 58 | ++++ | ++++ |
| 59 | ++++ | + |
| 60 | ++++ | ++++ |
| 61 | + | ++++ |
| 62 | ++++ | ++++ |
| 63 | ++++ | ++++ |
| 64 | ++++ | +++ |
| 65 | ++++ | +++ |
| 66 | + | + |
| 67 | ++++ | ++++ |
| 68 | ++++ | ++++ |
| 69 | + | + |
| 70 | +++ | +++ |
| 71 | ND | ND |
| 72 | ++++ | +++ |
| 73 | ++++ | +++ |
| 74 | ++++ | ++++ |
| 75 | ++++ | +++ |
| 76 | + | + |
| 77 | +++ | ++ |
| 78 | ++++ | +++ |
| 79 | ++++ | ++ |
| 80 | + + | + |
| 81 | + | + |
| 82 | ++++ | + |
| 83 | ++++ | ++++ |
| 84 | ++++ | ++ |
| 85 | ++++ | ++++ |
| 86 | ++++ | ++++ |
| 87 | ++++ | +++ |
| 88 | ++++ | + |
| 89 | +++ | + |

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 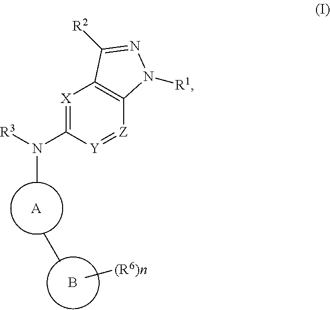
or a pharmaceutically acceptable salt, co-crystal, tautomer, stereoisomer, solvate, hydrate, polymorph, isotopically enriched compound, or prodrug thereof, wherein:
R¹ is hydrogen, substituted or unsubstituted alkyl, or a nitrogen protecting group;
R² is hydrogen, halogen, —CN, substituted or unsubstituted alkyl, or substituted or unsubstituted carbocyclyl;
X is CR⁷ or N;
Y is CR⁸ or N;
Z is CR⁹ or N;
R³ is hydrogen, substituted or unsubstituted alkyl, or a nitrogen protecting group;
A is 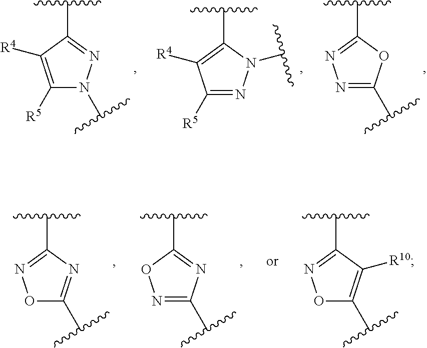
R⁴ is hydrogen, halogen, substituted or unsubstituted alkyl, substituted or unsubstituted carbocyclyl, substituted or unsubstituted aryl, or substituted or unsubstituted heteroaryl;
R⁵ is hydrogen, halogen, substituted or unsubstituted alkyl, substituted or unsubstituted carbocyclyl, substituted or unsubstituted aryl, or substituted or unsubstituted heteroaryl; or R⁴ and R⁵ together with the atoms to which they are attached form a substituted or unsubstituted carbocyclyl, substituted or unsubstituted aryl, substituted or unsubstituted heterocyclyl, or substituted or unsubstituted heteroaryl;
B is aryl, heterocyclyl, heteroaryl, or carbocyclyl;
each R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, oxo, —OR^{A}, —N₃, —N(R^{A})₂, —SR^{A}, —CN, —SCN, —C(═NR^{A})R^{A}, —C(═NR^{A})OR^{A}, —C(═NR^{A})N(R^{A})₂, —C(═O)R_{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A} or —S(O)₂R^{A};
n is 1, 2, 3, 4, or 5;
each R⁷, R⁸, and R⁹ is independently hydrogen, halogen, —CN, or substituted or unsubstituted alkyl;
R¹⁰ is hydrogen, halogen, substituted or unsubstituted alkyl, substituted or unsubstituted carbocyclyl, substituted or unsubstituted aryl, or substituted or unsubstituted heteroaryl; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

2. The compound of claim 1, or a pharmaceutically acceptable salt, co-crystal, tautomer, stereoisomer, solvate, hydrate, polymorph, isotopically enriched compound, or prodrug thereof, wherein R¹ is hydrogen or substituted or unsubstituted alkyl.

3. The compound of claim 1, or a pharmaceutically acceptable salt, co-crystal, tautomer, stereoisomer, solvate, hydrate, polymorph, isotopically enriched compound, or prodrug thereof, wherein R¹ is hydrogen.

4. The compound of any of claims 1-3, or a pharmaceutically acceptable salt, co-crystal, tautomer, stereoisomer, solvate, hydrate, polymorph, isotopically enriched compound, or prodrug thereof, wherein R² is hydrogen, halogen, or substituted or unsubstituted alkyl.

5. The compound of claim 4, or a pharmaceutically acceptable salt, co-crystal, tautomer, stereoisomer, solvate, hydrate, polymorph, isotopically enriched compound, or prodrug thereof, wherein R² is hydrogen or halogen.

6. The compound of claim 4, or a pharmaceutically acceptable salt, co-crystal, tautomer, stereoisomer, solvate, hydrate, polymorph, isotopically enriched compound, or prodrug thereof, wherein R² is hydrogen.

7. The compound of any of claims 1-6, or a pharmaceutically acceptable salt, co-crystal, tautomer, stereoisomer, solvate, hydrate, polymorph, isotopically enriched compound, or prodrug thereof, wherein R³ is hydrogen or substituted or unsubstituted alkyl.

8. The compound of claim 7, or a pharmaceutically acceptable salt, co-crystal, tautomer, stereoisomer, solvate, hydrate, polymorph, isotopically enriched compound, or prodrug thereof, wherein R³ is hydrogen.

9. The compound of any of claims 1-8, or a pharmaceutically acceptable salt, co-crystal, tautomer, stereoisomer, solvate, hydrate, polymorph, isotopically enriched compound, or prodrug thereof, wherein X is CR⁷.

10. The compound of claim 9, or a pharmaceutically acceptable salt, co-crystal, tautomer, stereoisomer, solvate, hydrate, polymorph, isotopically enriched compound, or prodrug thereof, wherein:
X is CR⁷; and
R⁷ is hydrogen or halogen, preferably hydrogen.

11. The compound of any of claims 1-10, or a pharmaceutically acceptable salt, co-crystal, tautomer, stereoisomer, solvate, hydrate, polymorph, isotopically enriched compound, or prodrug thereof, wherein Y is CR⁸.

12. The compound of any of claims 1-11, or a pharmaceutically acceptable salt, co-crystal, tautomer, stereoisomer, solvate, hydrate, polymorph, isotopically enriched compound, or prodrug thereof, wherein Z is CR⁹.

13. The compound of any of claims 1-8, or a pharmaceutically acceptable salt, co-crystal, tautomer, stereoisomer, solvate, hydrate, polymorph, isotopically enriched compound, or prodrug thereof, wherein:
X is CR⁷, wherein R⁷ is hydrogen or halogen, preferably hydrogen;
Y is CR⁸, wherein R⁸ is hydrogen; and
Z is CR⁹, wherein R⁹ is hydrogen.

14. The compound of any of claims 1-13, or a pharmaceutically acceptable salt, co-crystal, tautomer, stereoisomer, solvate, hydrate, polymorph, isotopically enriched compound, or prodrug thereof, wherein B is aryl, heterocyclyl, or heteroaryl.

15. The compound of claim 14, or a pharmaceutically acceptable salt, co-crystal, tautomer, stereoisomer, solvate, hydrate, polymorph, isotopically enriched compound, or prodrug thereof, wherein B is aryl or heteroaryl.

16. The compound of claim 14, or a pharmaceutically acceptable salt, co-crystal, tautomer, stereoisomer, solvate, hydrate, polymorph, isotopically enriched compound, or prodrug thereof, wherein B is phenyl or pyridinyl.

17. The compound of claim 14, or a pharmaceutically acceptable salt, co-crystal, tautomer, stereoisomer, solvate, hydrate, polymorph, isotopically enriched compound, or prodrug thereof, wherein B is phenyl.

18. The compound of claim 14, or a pharmaceutically acceptable salt, co-crystal, tautomer, stereoisomer, solvate, hydrate, polymorph, isotopically enriched compound, or prodrug thereof, wherein B is pyridinyl.

19. The compound of any of claims 1-18, or a pharmaceutically acceptable salt, co-crystal, tautomer, stereoisomer, solvate, hydrate, polymorph, isotopically enriched compound, or prodrug thereof, wherein n is 1 or 2.

20. The compound of claim 19, or a pharmaceutically acceptable salt, co-crystal, tautomer, stereoisomer, solvate, hydrate, polymorph, isotopically enriched compound, or prodrug thereof, wherein n is 1.

21. The compound of claim 19, or a pharmaceutically acceptable salt, co-crystal, tautomer, stereoisomer, solvate, hydrate, polymorph, isotopically enriched compound, or prodrug thereof, wherein n is 2.

22. The compound of any of claims 1-18, or a pharmaceutically acceptable salt, co-crystal, tautomer, stereoisomer, solvate, hydrate, polymorph, isotopically enriched compound, or prodrug thereof, wherein —B(R⁶)ₙ is

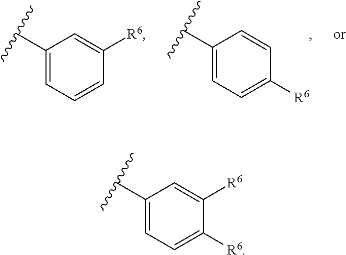

23. The compound of claim 22, or a pharmaceutically acceptable salt, co-crystal, tautomer, stereoisomer, solvate, hydrate, polymorph, isotopically enriched compound, or prodrug thereof,
wherein —B(R⁶)ₙ is

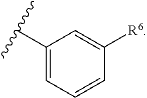

24. The compound of any of claims 1-18, or a pharmaceutically acceptable salt, co-crystal, tautomer, stereoisomer, solvate, hydrate, polymorph, isotopically enriched compound, or prodrug thereof, wherein —B(R⁶)ₙ is

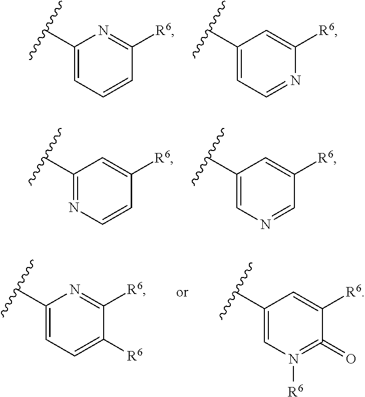

25. The compound of any of claims 1-18, or a pharmaceutically acceptable salt, co-crystal, tautomer, stereoisomer, solvate, hydrate, polymorph, isotopically enriched compound, or prodrug thereof, wherein —B(R⁶)ₙ is

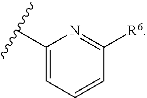

26. The compound of any of claims 1-15, or a pharmaceutically acceptable salt, co-crystal, tautomer, stereoisomer, solvate, hydrate, polymorph, isotopically enriched compound, or prodrug thereof, wherein —B(R⁶)ₙ is 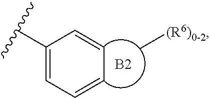
wherein B2 is 5-6-membered, monocyclic, unsubstituted heteroaryl, or 5-6-membered, monocyclic, unsubstituted heterocyclyl, and R⁶ is directly attached to B2.

27. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently halogen, substituted or unsubstituted alkyl, substituted or unsubstituted aryl, substituted or unsubstituted heteroaryl, substituted or unsubstituted heteroaliphatic, oxo, —OR^{A}, —N(R^{A})₂, —CN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}.

28. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently substituted or unsubstituted alkyl, substituted or unsubstituted heteroaryl, oxo, —OR^{A}, —N(R^{A})₂, or NR^{A}C(═O)R^{A}.

29. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently substituted or unsubstituted alkyl, substituted or unsubstituted heteroaryl, oxo, —OC₁₋₄alkyl, —OCH₂C(═O)N(R^{A})₂, —N(R^{A})₂, or —NR^{A}C(═O)R^{A}.

30. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently C₁₋₄ alkyl, substituted or unsubstituted heteroaryl, oxo, —OC₁₋₄ alkyl, —OCH₂C(═O)NHC₁₋₄ alkyl, —NH₂, —NHC(═O)aryl, or —NHC(═O)heteroaryl, wherein each alkyl, aryl, and heteroaryl are substituted or unsubstituted.

31. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently each R⁶ is independently oxo, —CH₃, —OCH₃, —F,

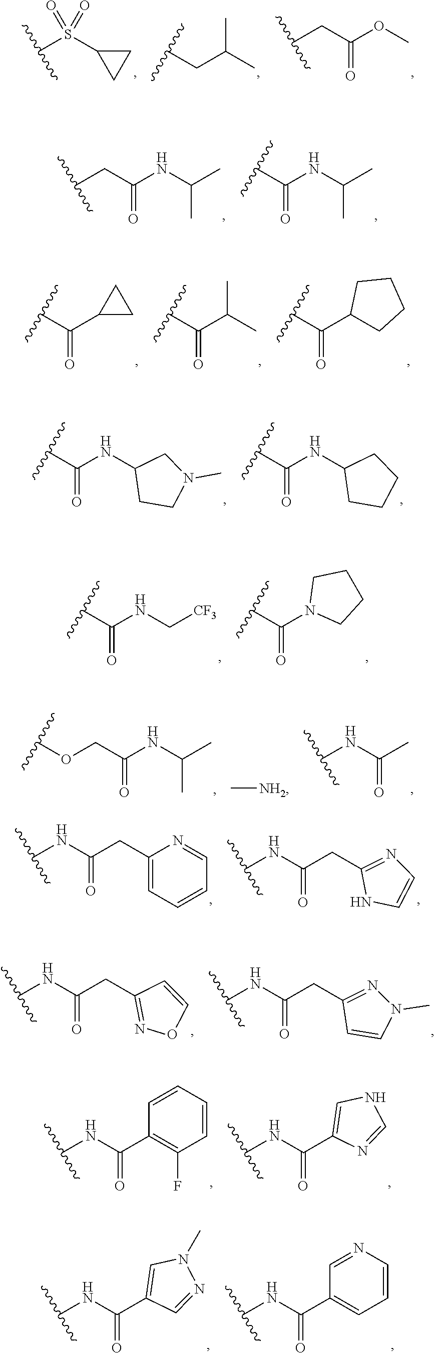

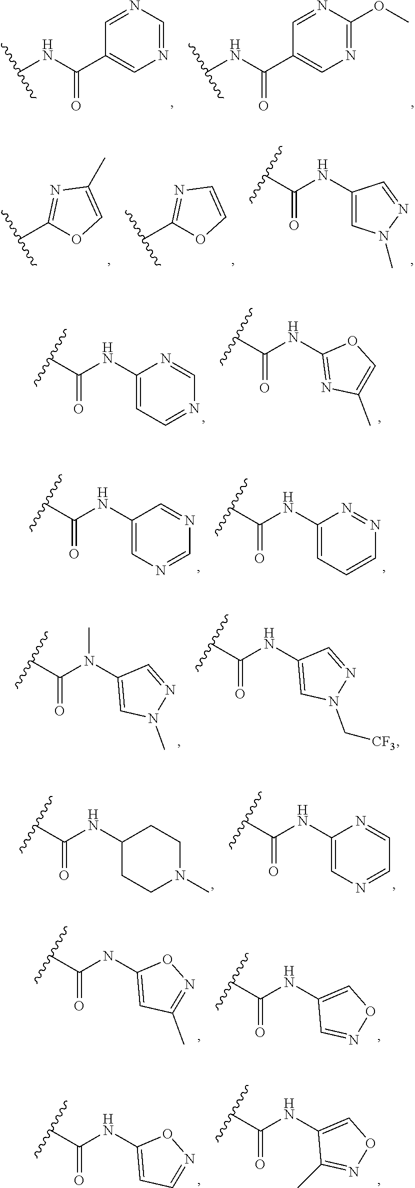

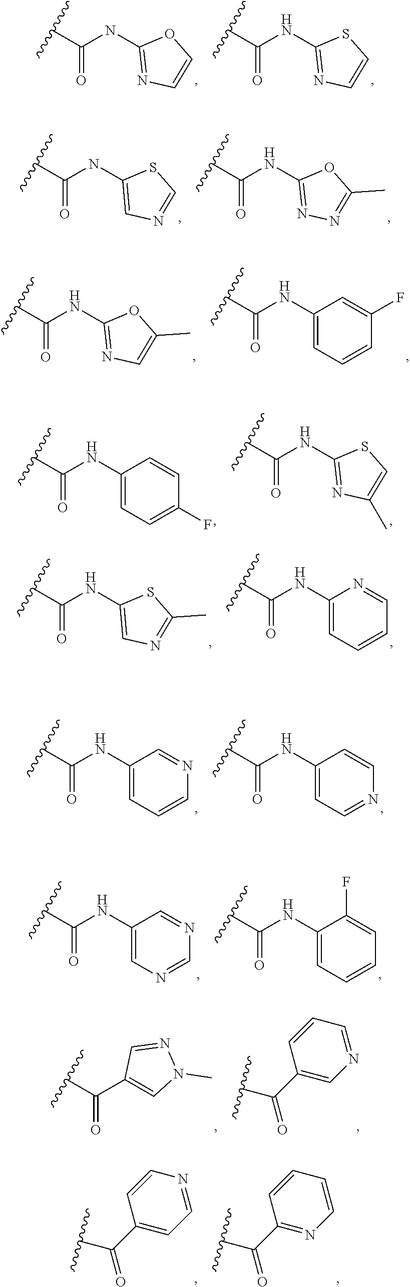

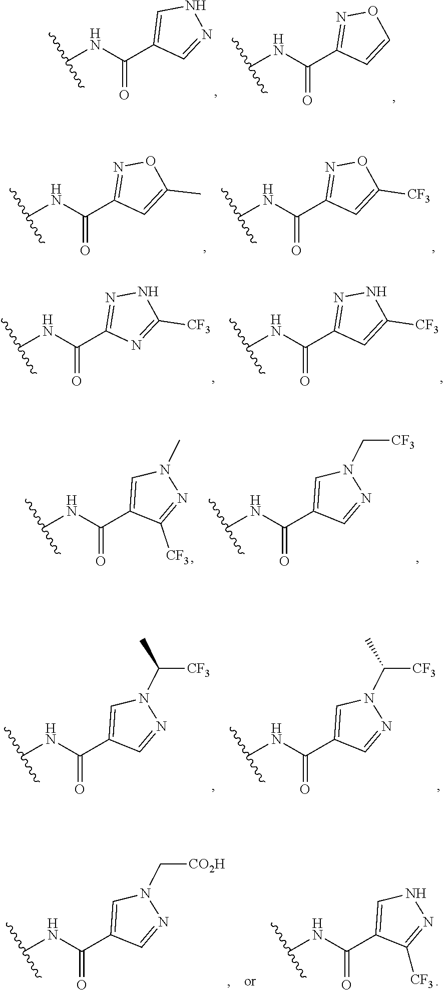

32. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently oxo, —CH₃, —OCH₃, —F,

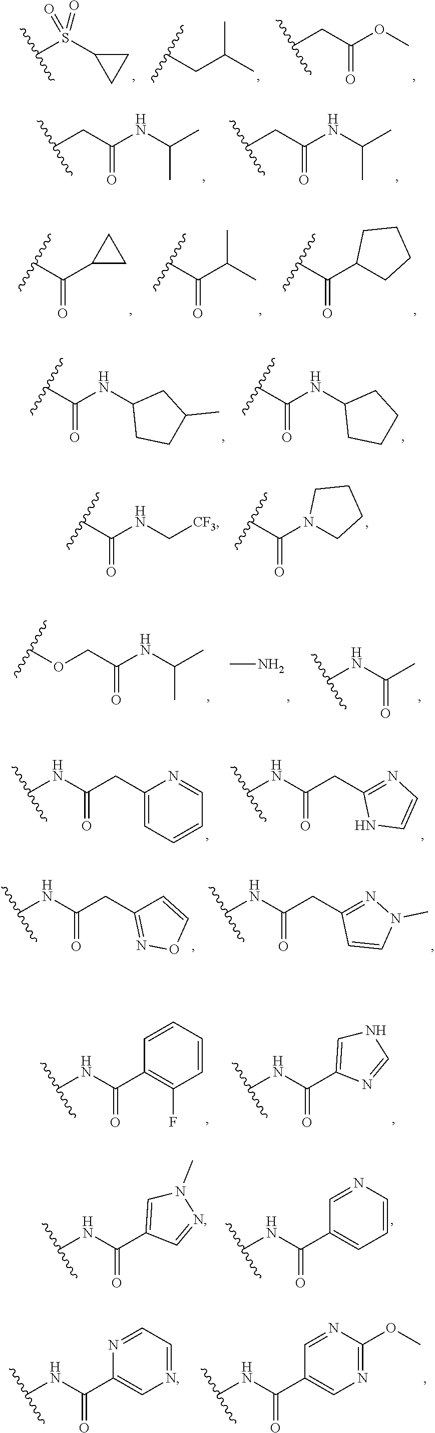

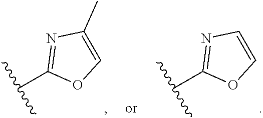

33. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently oxo, —CH₃, —OCH₃,

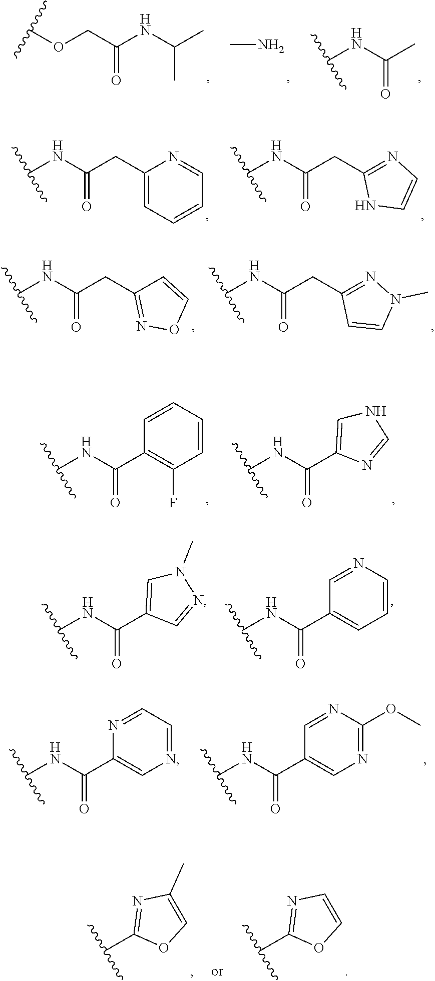

34. The compound of any of claims 1-26, or a pharmaceutically acceptable salt, co-crystal, tautomer, stereoisomer, solvate, hydrate, polymorph, isotopically enriched compound, or prodrug thereof, wherein each R⁶ is independently —OCH₃

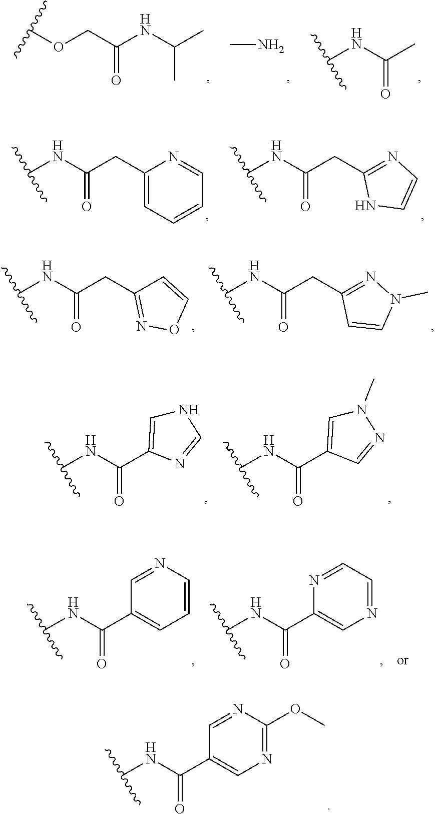

35. The compound of any of claims 1-26, or a pharmaceutically acceptable salt, co-crystal, tautomer, stereoisomer, solvate, hydrate, polymorph, isotopically enriched compound, or prodrug thereof, wherein R⁶ is

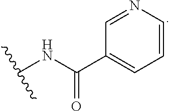 [SMILES: *NC(=O)c1cccnc1]

36. The compound of any of claims 1-26, or a pharmaceutically acceptable salt, co-crystal, tautomer, stereoisomer, solvate, hydrate, polymorph, isotopically enriched compound, or prodrug thereof, wherein R⁶ is

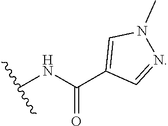 [SMILES: *NC(=O)c1cnn(C)c1]

37. The compound of any of claims 1-36, or a pharmaceutically acceptable salt, co-crystal, tautomer, stereoisomer, solvate, hydrate, polymorph, isotopically enriched compound, or prodrug thereof, wherein A is,

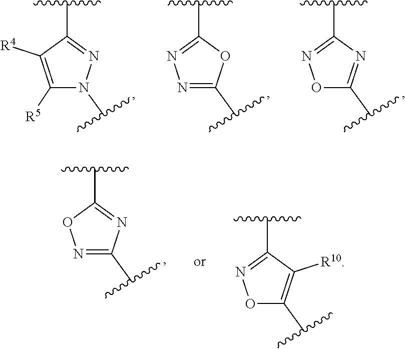

38. The compound of any of claims 1-37, or a pharmaceutically acceptable salt, co-crystal, tautomer, stereoisomer, solvate, hydrate, polymorph, isotopically enriched compound, or prodrug thereof, wherein A is

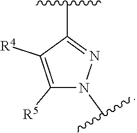

39. The compound of any of claims 1-38, or a pharmaceutically acceptable salt, co-crystal, tautomer, stereoisomer, solvate, hydrate, polymorph, isotopically enriched compound, or prodrug thereof, wherein R⁴ is hydrogen, halogen, or substituted or unsubstituted alkyl.

40. The compound of claim 39, or a pharmaceutically acceptable salt, co-crystal, tautomer, stereoisomer, solvate, hydrate, polymorph, isotopically enriched compound, or prodrug thereof, wherein R⁴ is hydrogen.

41. The compound of claim 39, or a pharmaceutically acceptable salt, co-crystal, tautomer, stereoisomer, solvate, hydrate, polymorph, isotopically enriched compound, or prodrug thereof, wherein R⁴ is substituted or unsubstituted alkyl.

42. The compound of any of claims 1-41, or a pharmaceutically acceptable salt, co-crystal, tautomer, stereoisomer, solvate, hydrate, polymorph, isotopically enriched compound, or prodrug thereof, wherein R⁵ is hydrogen, halogen, or substituted or unsubstituted alkyl.

43. The compound of claim 42, or a pharmaceutically acceptable salt, co-crystal, tautomer, stereoisomer, solvate, hydrate, polymorph, isotopically enriched compound, or prodrug thereof, wherein R⁵ is hydrogen.

44. The compound of claim 42, or a pharmaceutically acceptable salt, co-crystal, tautomer, stereoisomer, solvate, hydrate, polymorph, isotopically enriched compound, or prodrug thereof, wherein R⁵ is substituted or unsubstituted alkyl.

45. The compound of any of claims 1-38, or a pharmaceutically acceptable salt, co-crystal, tautomer, stereoisomer, solvate, hydrate, polymorph, isotopically enriched compound, or prodrug thereof, wherein:
R⁴ is hydrogen or substituted or unsubstituted alkyl; and
R⁵ is hydrogen or substituted or unsubstituted alkyl; or R⁴ and R⁵ together with the atoms to which they are attached form a substituted or unsubstituted aryl ring.

46. The compound of any of claims 1-38, or a pharmaceutically acceptable salt, co-crystal, tautomer, stereoisomer, solvate, hydrate, polymorph, isotopically enriched compound, or prodrug thereof, wherein R⁴ and R⁵ together with the atoms to which they are attached form a substituted or unsubstituted aryl ring.

47. The compound of any of claims 1-46, or a pharmaceutically acceptable salt, co-crystal, tautomer, stereoisomer, solvate, hydrate, polymorph, isotopically enriched compound, or prodrug thereof, wherein A is

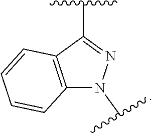 [SMILES: *c1nn(*)c2ccccc12]

48. The compound of any of claims 1-37, or a pharmaceutically acceptable salt, co-crystal, tautomer, stereoisomer, solvate, hydrate, polymorph, isotopically enriched compound, or prodrug thereof, wherein A is

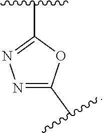 [SMILES: *c1nnc(*)o1]

49. The compound of any of claims 1-37, or a pharmaceutically acceptable salt, co-crystal, tautomer, stereoisomer, solvate, hydrate, polymorph, isotopically enriched compound, or prodrug thereof, wherein A is

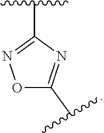 [SMILES: *c1noc(*)n1]

50. The compound of any of claims 1-37, or a pharmaceutically acceptable salt, co-crystal, tautomer, stereoisomer, solvate, hydrate, polymorph, isotopically enriched compound, or prodrug thereof, wherein A is

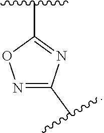 [SMILES: *c1noc(*)n1]

51. The compound of any of claims 1-37, or a pharmaceutically acceptable salt, co-crystal, tautomer, stereoisomer, solvate, hydrate, polymorph, isotopically enriched compound, or prodrug thereof, wherein A is

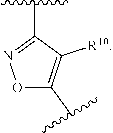

52. The compound of any of claims 1-37 and 51, or a pharmaceutically acceptable salt, co-crystal, tautomer, stereoisomer, solvate, hydrate, polymorph, isotopically enriched compound, or prodrug thereof, wherein R¹⁰ is hydrogen, halogen, or substituted or unsubstituted alkyl.

53. The compound of claim 52, or a pharmaceutically acceptable salt, co-crystal, tautomer, stereoisomer, solvate, hydrate, polymorph, isotopically enriched compound, or prodrug thereof, wherein R¹⁰ is hydrogen.

54. The compound of any of claims 1-53, wherein the compound is of Formula (I-a): 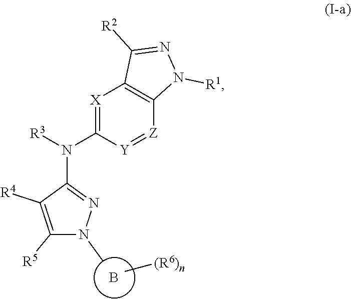
or a pharmaceutically acceptable salt, co-crystal, tautomer, stereoisomer, solvate, hydrate, polymorph, isotopically enriched compound, or prodrug thereof.

55. The compound of any of claims 1-53, wherein the compound is of Formula (I-a-1): 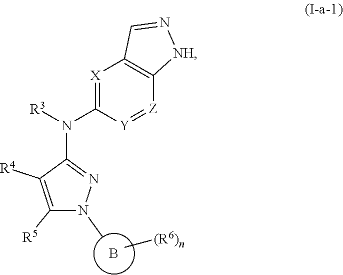
or a pharmaceutically acceptable salt, co-crystal, tautomer, stereoisomer, solvate, hydrate, polymorph, isotopically enriched compound, or prodrug thereof.

56. The compound of any of claims 1-53, wherein the compound is of Formula (I-a-2): 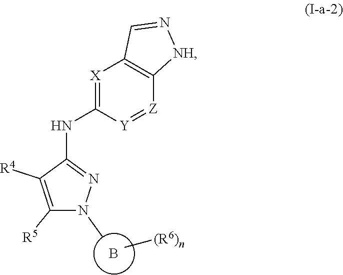
or a pharmaceutically acceptable salt, co-crystal, tautomer, stereoisomer, solvate, hydrate, polymorph, isotopically enriched compound, or prodrug thereof.

57. The compound of any of claims 1-53, wherein the compound is of Formula (I-a-3): 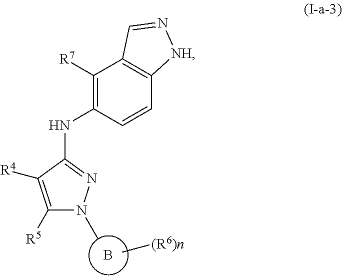
or a pharmaceutically acceptable salt, co-crystal, tautomer, stereoisomer, solvate, hydrate, polymorph, isotopically enriched compound, or prodrug thereof.

58. The compound of any of claims 1-53, wherein the compound is of Formula (I-a-4): 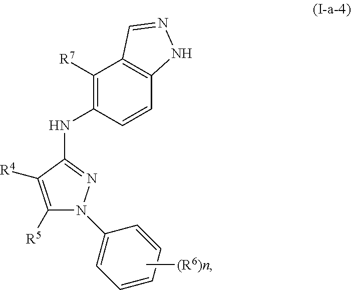
or a pharmaceutically acceptable salt, co-crystal, tautomer, stereoisomer, solvate, hydrate, polymorph, isotopically enriched compound, or prodrug thereof.

59. The compound of any of claims 1-53, wherein the compound is of Formula (I-a-4a): 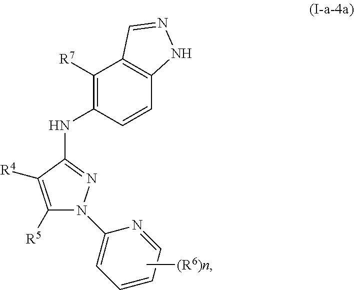
or a pharmaceutically acceptable salt, co-crystal, tautomer, stereoisomer, solvate, hydrate, polymorph, isotopically enriched compound, or prodrug thereof.

60. The compound of any of claims 1-53, wherein the compound is of Formula (I-a-5): 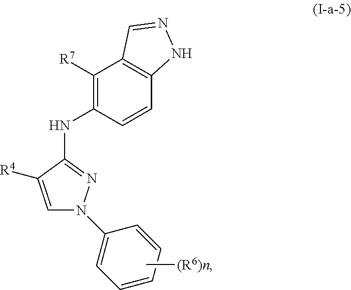
or a pharmaceutically acceptable salt, co-crystal, tautomer, stereoisomer, solvate, hydrate, polymorph, isotopically enriched compound, or prodrug thereof.

61. The compound of any of claims 1-53, wherein the compound is of Formula (I-a-5a): 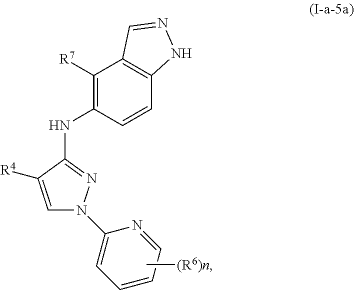
or a pharmaceutically acceptable salt, co-crystal, tautomer, stereoisomer, solvate, hydrate, polymorph, isotopically enriched compound, or prodrug thereof.

62. The compound of any of claims 1-53, wherein the compound is of Formula (I-a-6): 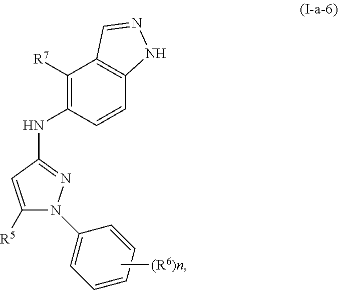
or a pharmaceutically acceptable salt, co-crystal, tautomer, stereoisomer, solvate, hydrate, polymorph, isotopically enriched compound, or prodrug thereof.

63. The compound of any of claims 1-53, wherein the compound is of Formula (I-a-6a): 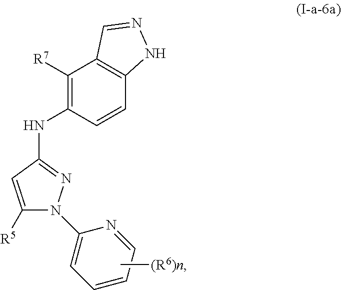
or a pharmaceutically acceptable salt, co-crystal, tautomer, stereoisomer, solvate, hydrate, polymorph, isotopically enriched compound, or prodrug thereof.

64. The compound of any of claims 1-53, wherein the compound is of Formula (I-a-7): 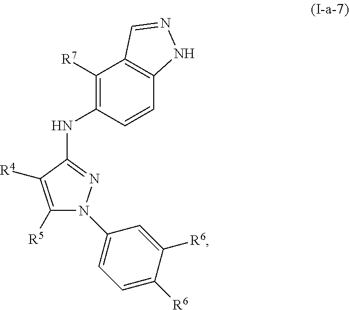
or a pharmaceutically acceptable salt, co-crystal, tautomer, stereoisomer, solvate, hydrate, polymorph, isotopically enriched compound, or prodrug thereof.

65. The compound of any of claims 1-53, wherein the compound is of Formula (I-a-8): 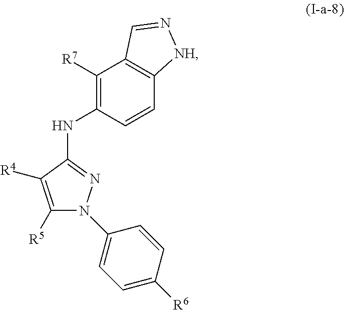
or a pharmaceutically acceptable salt, co-crystal, tautomer, stereoisomer, solvate, hydrate, polymorph, isotopically enriched compound, or prodrug thereof.

66. The compound of any of claims 1-53, wherein the compound is of Formula (I-a-9): 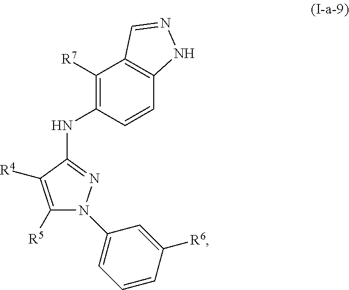
or a pharmaceutically acceptable salt, co-crystal, tautomer, stereoisomer, solvate, hydrate, polymorph, isotopically enriched compound, or prodrug thereof.

67. The compound of any of claims 1-53, wherein the compound is of Formula (I-a-9a): 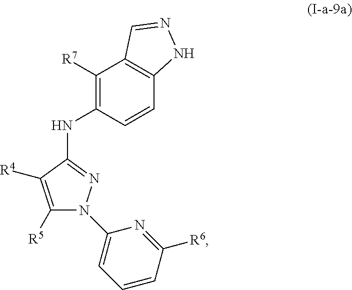
or a pharmaceutically acceptable salt, co-crystal, tautomer, stereoisomer, solvate, hydrate, polymorph, isotopically enriched compound, or prodrug thereof.

68. The compound of any of claims 1-53, wherein the compound is of Formula (I-a-10): 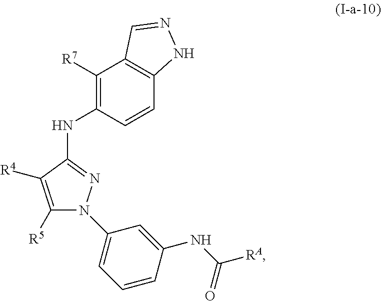
or a pharmaceutically acceptable salt, co-crystal, tautomer, stereoisomer, solvate, hydrate, polymorph, isotopically enriched compound, or prodrug thereof.

69. The compound of any of claims 1-53, wherein the compound is of Formula (I-a-10a): 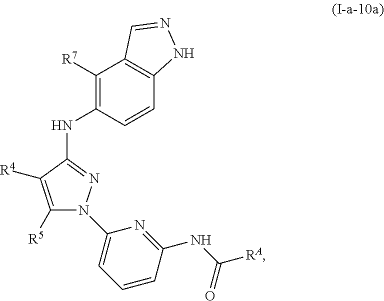
or a pharmaceutically acceptable salt, co-crystal, tautomer, stereoisomer, solvate, hydrate, polymorph, isotopically enriched compound, or prodrug thereof.

70. The compound of any of claims 1-53, wherein the compound is of Formula (I-b): 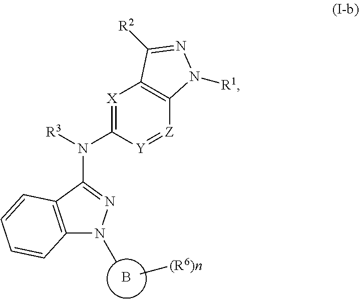
or a pharmaceutically acceptable salt, co-crystal, tautomer, stereoisomer, solvate, hydrate, polymorph, isotopically enriched compound, or prodrug thereof.

71. The compound of any of claims 1-53, wherein the compound is of Formula (I-b-1): 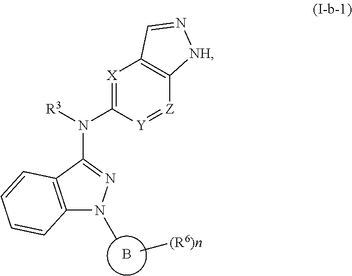
or a pharmaceutically acceptable salt, co-crystal, tautomer, stereoisomer, solvate, hydrate, polymorph, isotopically enriched compound, or prodrug thereof.

72. The compound of any of claims 1-53, wherein the compound is of Formula (I-b-2): 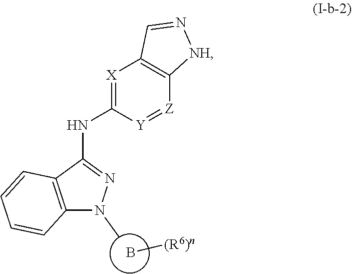
or a pharmaceutically acceptable salt, co-crystal, tautomer, stereoisomer, solvate, hydrate, polymorph, isotopically enriched compound, or prodrug thereof.

73. The compound of any of claims 1-53, wherein the compound is of Formula (I-b-3): 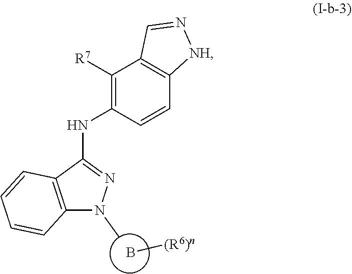
or a pharmaceutically acceptable salt, co-crystal, tautomer, stereoisomer, solvate, hydrate, polymorph, isotopically enriched compound, or prodrug thereof.

74. The compound of any of claims 1-53, wherein the compound is of Formula (I-b-4): 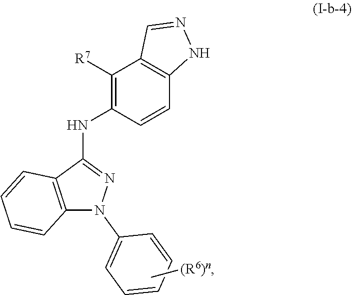
or a pharmaceutically acceptable salt, co-crystal, tautomer, stereoisomer, solvate, hydrate, polymorph, isotopically enriched compound, or prodrug thereof.

75. The compound of any of claims 1-53, wherein the compound is of Formula (I-b-4a): 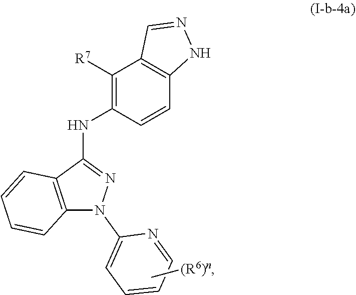
or a pharmaceutically acceptable salt, co-crystal, tautomer, stereoisomer, solvate, hydrate, polymorph, isotopically enriched compound, or prodrug thereof.

76. The compound of any of claims 1-53, wherein the compound is of Formula (I-b-5): 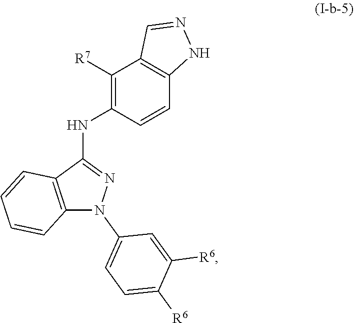
or a pharmaceutically acceptable salt, co-crystal, tautomer, stereoisomer, solvate, hydrate, polymorph, isotopically enriched compound, or prodrug thereof.

77. The compound of any of claims 1-53, wherein the compound is of Formula (I-b-5a): 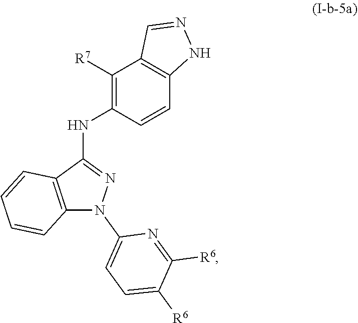
or a pharmaceutically acceptable salt, co-crystal, tautomer, stereoisomer, solvate, hydrate, polymorph, isotopically enriched compound, or prodrug thereof.

78. The compound of any of claims 1-53, wherein the compound is of Formula (I-b-6): 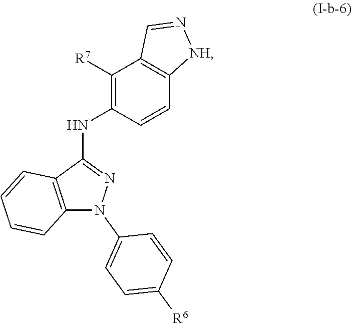
or a pharmaceutically acceptable salt, co-crystal, tautomer, stereoisomer, solvate, hydrate, polymorph, isotopically enriched compound, or prodrug thereof.

79. The compound of any of claims 1-53, wherein the compound is of Formula (I-b-6a): 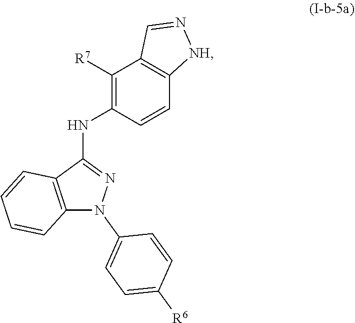
or a pharmaceutically acceptable salt, co-crystal, tautomer, stereoisomer, solvate, hydrate, polymorph, isotopically enriched compound, or prodrug thereof.

80. The compound of any of claims 1-53, wherein the compound is of Formula (I-b-7): 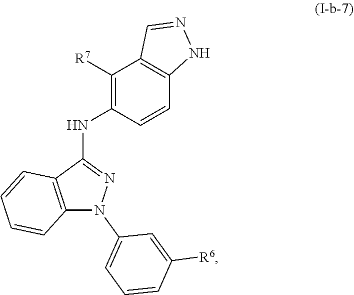
or a pharmaceutically acceptable salt, co-crystal, tautomer, stereoisomer, solvate, hydrate, polymorph, isotopically enriched compound, or prodrug thereof.

81. The compound of any of claims 1-53, wherein the compound is of Formula (I-b-7a): 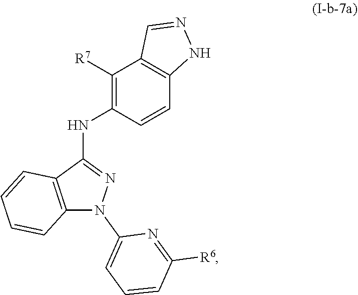
or a pharmaceutically acceptable salt, co-crystal, tautomer, stereoisomer, solvate, hydrate, polymorph, isotopically enriched compound, or prodrug thereof.

82. The compound of any of claims 1-53, wherein the compound is of Formula (I-b-8): 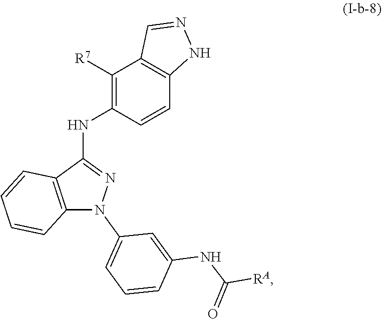
or a pharmaceutically acceptable salt, co-crystal, tautomer, stereoisomer, solvate, hydrate, polymorph, isotopically enriched compound, or prodrug thereof.

83. The compound of any of claims 1-53, wherein the compound is of Formula (I-b-8a): 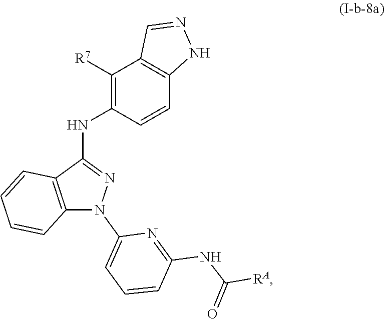
or a pharmaceutically acceptable salt, co-crystal, tautomer, stereoisomer, solvate, hydrate, polymorph, isotopically enriched compound, or prodrug thereof.

84. The compound of any of claims 1-53, wherein the compound is of Formula (I-c): 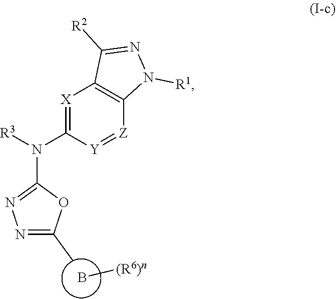
or a pharmaceutically acceptable salt, co-crystal, tautomer, stereoisomer, solvate, hydrate, polymorph, isotopically enriched compound, or prodrug thereof.

85. The compound of any of claims 1-53, wherein the compound is of Formula (I-c-1): 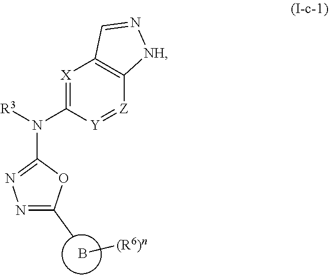
or a pharmaceutically acceptable salt, co-crystal, tautomer, stereoisomer, solvate, hydrate, polymorph, isotopically enriched compound, or prodrug thereof.

86. The compound of any of claims 1-53, wherein the compound is of Formula (I-c-2): 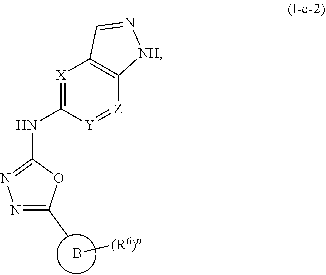
or a pharmaceutically acceptable salt, co-crystal, tautomer, stereoisomer, solvate, hydrate, polymorph, isotopically enriched compound, or prodrug thereof.

87. The compound of any of claims 1-53, wherein the compound is of Formula (I-c-3): 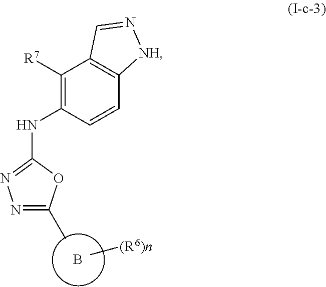
or a pharmaceutically acceptable salt, co-crystal, tautomer, stereoisomer, solvate, hydrate, polymorph, isotopically enriched compound, or prodrug thereof.

88. The compound of any of claims 1-53, wherein the compound is of Formula (I-c-4): 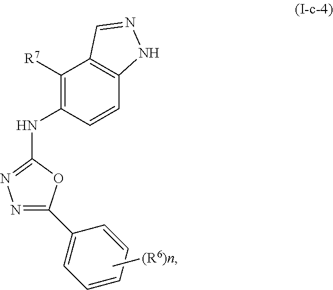
or a pharmaceutically acceptable salt, co-crystal, tautomer, stereoisomer, solvate, hydrate, polymorph, isotopically enriched compound, or prodrug thereof.

89. The compound of any of claims 1-53, wherein the compound is of Formula (I-c-4a): 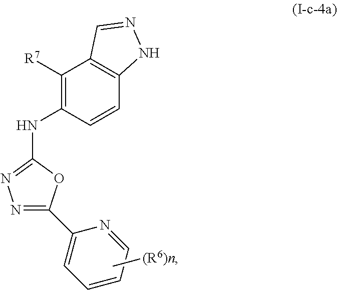
or a pharmaceutically acceptable salt, co-crystal, tautomer, stereoisomer, solvate, hydrate, polymorph, isotopically enriched compound, or prodrug thereof.

90. The compound of any of claims 1-53, wherein the compound is of Formula (I-c-5): 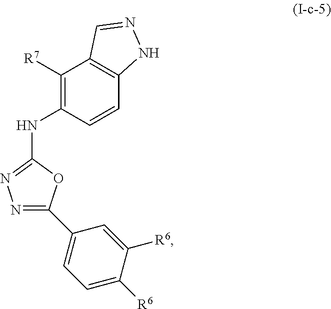
or a pharmaceutically acceptable salt, co-crystal, tautomer, stereoisomer, solvate, hydrate, polymorph, isotopically enriched compound, or prodrug thereof.

91. The compound of any of claims 1-53, wherein the compound is of Formula (I-c-6): 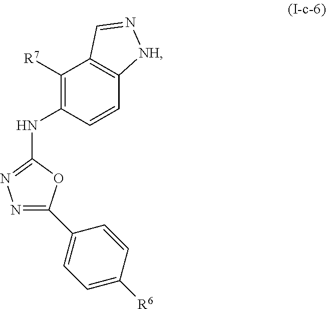
or a pharmaceutically acceptable salt, co-crystal, tautomer, stereoisomer, solvate, hydrate, polymorph, isotopically enriched compound, or prodrug thereof.

92. The compound of any of claims 1-53, wherein the compound is of Formula (I-c-7): 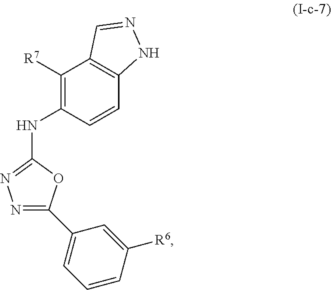
or a pharmaceutically acceptable salt, co-crystal, tautomer, stereoisomer, solvate, hydrate, polymorph, isotopically enriched compound, or prodrug thereof.

93. The compound of any of claims 1-53, wherein the compound is of Formula (I-c-7a): 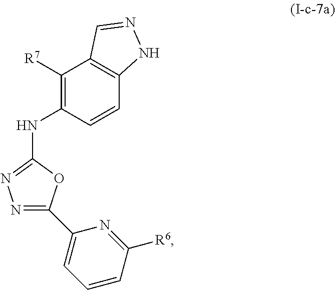
or a pharmaceutically acceptable salt, co-crystal, tautomer, stereoisomer, solvate, hydrate, polymorph, isotopically enriched compound, or prodrug thereof.

94. The compound of any of claims 1-53, wherein the compound is of Formula (I-c-8): 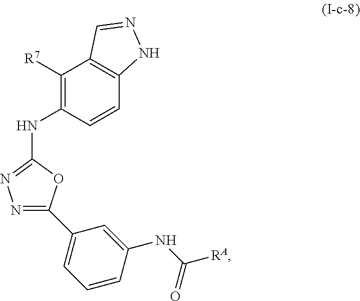
or a pharmaceutically acceptable salt, co-crystal, tautomer, stereoisomer, solvate, hydrate, polymorph, isotopically enriched compound, or prodrug thereof.

95. The compound of any of claims 1-53, wherein the compound is of Formula (I-c-8a): 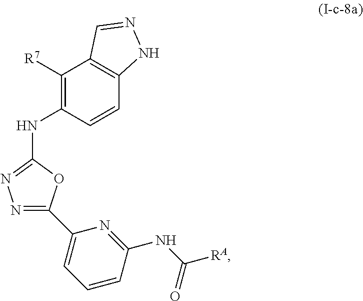
or a pharmaceutically acceptable salt, co-crystal, tautomer, stereoisomer, solvate, hydrate, polymorph, isotopically enriched compound, or prodrug thereof.

96. The compound of any of claims 1-53, wherein the compound is of Formula (I-d): 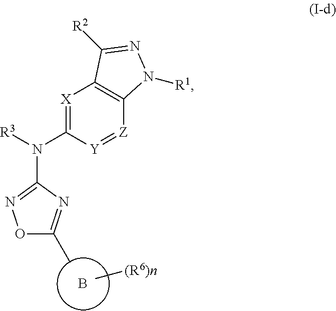
or a pharmaceutically acceptable salt, co-crystal, tautomer, stereoisomer, solvate, hydrate, polymorph, isotopically enriched compound, or prodrug thereof.

97. The compound of any of claims 1-53, wherein the compound is of Formula (I-d-1): 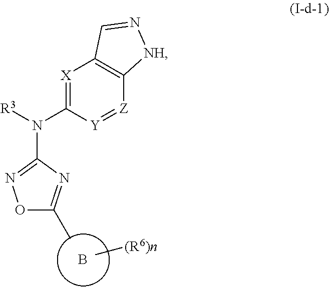
or a pharmaceutically acceptable salt, co-crystal, tautomer, stereoisomer, solvate, hydrate, polymorph, isotopically enriched compound, or prodrug thereof.

98. The compound of any of claims 1-53, wherein the compound is of Formula (I-d-2): 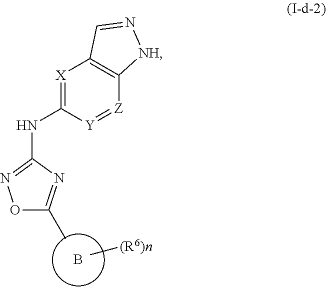
or a pharmaceutically acceptable salt, co-crystal, tautomer, stereoisomer, solvate, hydrate, polymorph, isotopically enriched compound, or prodrug thereof.

99. The compound of any of claims 1-53, wherein the compound is of Formula (I-d-3): 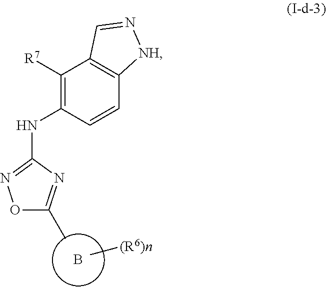
or a pharmaceutically acceptable salt, co-crystal, tautomer, stereoisomer, solvate, hydrate, polymorph, isotopically enriched compound, or prodrug thereof.

100. The compound of any of claims 1-53, wherein the compound is of Formula (I-d-4): 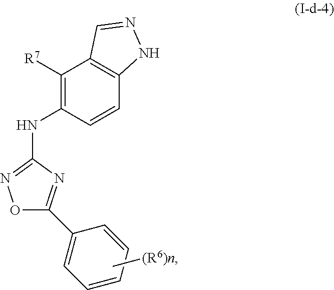
or a pharmaceutically acceptable salt, co-crystal, tautomer, stereoisomer, solvate, hydrate, polymorph, isotopically enriched compound, or prodrug thereof.

101. The compound of any of claims 1-53, wherein the compound is of Formula (I-d-4a): 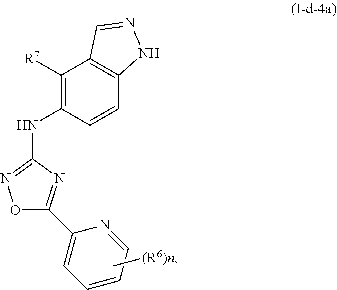
or a pharmaceutically acceptable salt, co-crystal, tautomer, stereoisomer, solvate, hydrate, polymorph, isotopically enriched compound, or prodrug thereof.

102. The compound of any of claims 1-53, wherein the compound is of Formula (I-d-5): 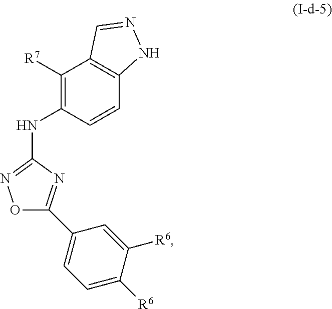
or a pharmaceutically acceptable salt, co-crystal, tautomer, stereoisomer, solvate, hydrate, polymorph, isotopically enriched compound, or prodrug thereof.

103. The compound of any of claims 1-53, wherein the compound is of Formula (I-d-6): 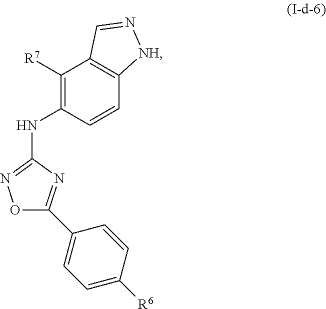
or a pharmaceutically acceptable salt, co-crystal, tautomer, stereoisomer, solvate, hydrate, polymorph, isotopically enriched compound, or prodrug thereof.

104. The compound of any of claims 1-53, wherein the compound is of Formula (I-d-7): 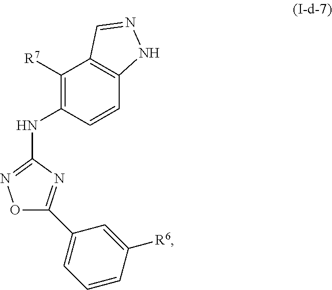
or a pharmaceutically acceptable salt, co-crystal, tautomer, stereoisomer, solvate, hydrate, polymorph, isotopically enriched compound, or prodrug thereof.

105. The compound of any of claims 1-53, wherein the compound is of Formula (I-d-7a): 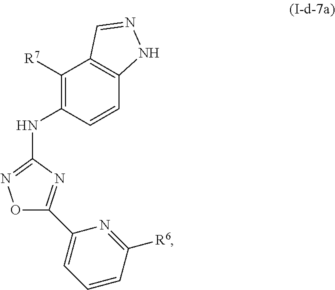
or a pharmaceutically acceptable salt, co-crystal, tautomer, stereoisomer, solvate, hydrate, polymorph, isotopically enriched compound, or prodrug thereof.

106. The compound of any of claims 1-53, wherein the compound is of Formula (I-d-8): 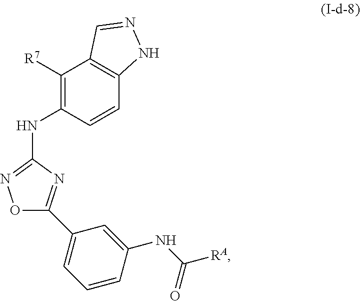
or a pharmaceutically acceptable salt, co-crystal, tautomer, stereoisomer, solvate, hydrate, polymorph, isotopically enriched compound, or prodrug thereof.

107. The compound of any of claims 1-53, wherein the compound is of Formula (I-d-8a): 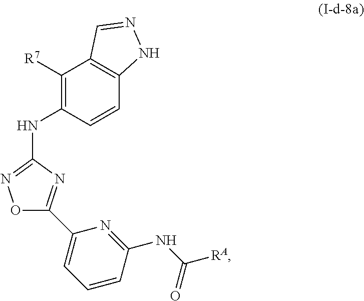
or a pharmaceutically acceptable salt, co-crystal, tautomer, stereoisomer, solvate, hydrate, polymorph, isotopically enriched compound, or prodrug thereof.

108. The compound of any of claims 1-53, wherein the compound is of Formula (I-e): 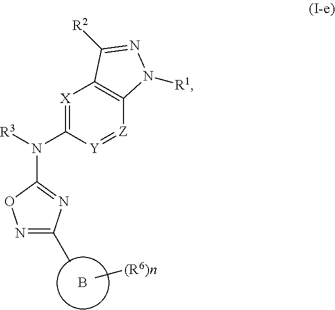
or a pharmaceutically acceptable salt, co-crystal, tautomer, stereoisomer, solvate, hydrate, polymorph, isotopically enriched compound, or prodrug thereof.

109. The compound of any of claims 1-53, wherein the compound is of Formula (I-e-1): 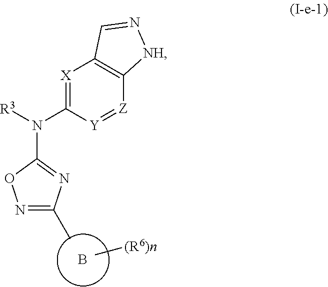
or a pharmaceutically acceptable salt, co-crystal, tautomer, stereoisomer, solvate, hydrate, polymorph, isotopically enriched compound, or prodrug thereof.

110. The compound of any of claims 1-53, wherein the compound is of Formula (I-e-2): 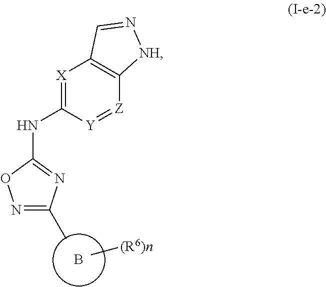
or a pharmaceutically acceptable salt, co-crystal, tautomer, stereoisomer, solvate, hydrate, polymorph, isotopically enriched compound, or prodrug thereof.

111. The compound of any of claims 1-53, wherein the compound is of Formula (I-e-3): 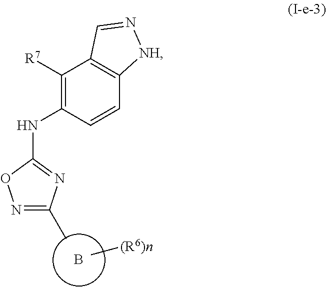
or a pharmaceutically acceptable salt, co-crystal, tautomer, stereoisomer, solvate, hydrate, polymorph, isotopically enriched compound, or prodrug thereof.

112. The compound of any of claims 1-53, wherein the compound is of Formula (I-e-4): 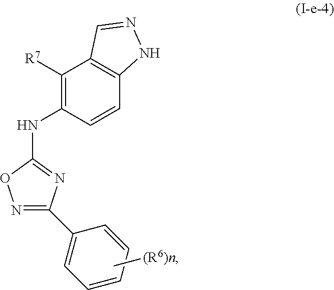
or a pharmaceutically acceptable salt, co-crystal, tautomer, stereoisomer, solvate, hydrate, polymorph, isotopically enriched compound, or prodrug thereof.

113. The compound of any of claims 1-53, wherein the compound is of Formula (I-e-4a): 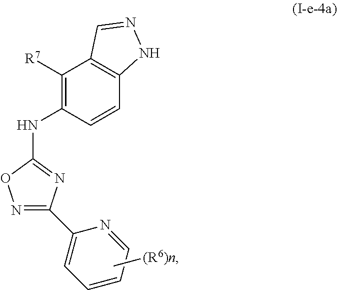
or a pharmaceutically acceptable salt, co-crystal, tautomer, stereoisomer, solvate, hydrate, polymorph, isotopically enriched compound, or prodrug thereof.

114. The compound of any of claims 1-53, wherein the compound is of Formula (I-e-5): 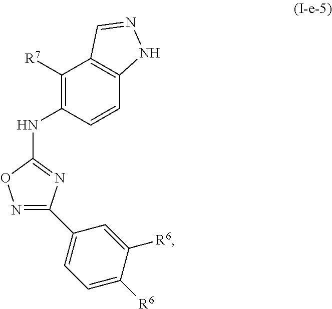
or a pharmaceutically acceptable salt, co-crystal, tautomer, stereoisomer, solvate, hydrate, polymorph, isotopically enriched compound, or prodrug thereof.

115. The compound of any of claims 1-53, wherein the compound is of Formula (I-e-6): 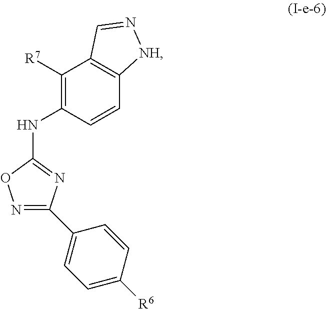
or a pharmaceutically acceptable salt, co-crystal, tautomer, stereoisomer, solvate, hydrate, polymorph, isotopically enriched compound, or prodrug thereof.

116. The compound of any of claims 1-53, wherein the compound is of Formula (I-e-7): 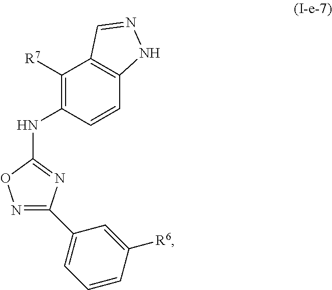
or a pharmaceutically acceptable salt, co-crystal, tautomer, stereoisomer, solvate, hydrate, polymorph, isotopically enriched compound, or prodrug thereof.

117. The compound of any of claims 1-53, wherein the compound is of Formula (I-e-7a): 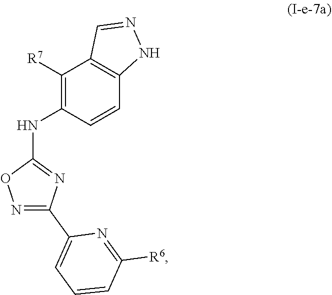
or a pharmaceutically acceptable salt, co-crystal, tautomer, stereoisomer, solvate, hydrate, polymorph, isotopically enriched compound, or prodrug thereof.

118. The compound of any of claims 1-53, wherein the compound is of Formula (I-e-8): 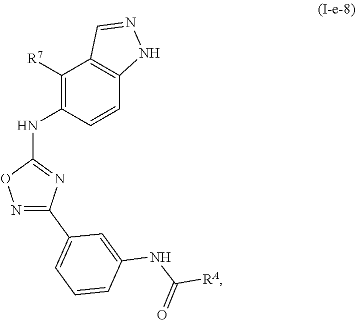
or a pharmaceutically acceptable salt, co-crystal, tautomer, stereoisomer, solvate, hydrate, polymorph, isotopically enriched compound, or prodrug thereof.

119. The compound of any of claims 1-53, wherein the compound is of Formula (I-e-8a): 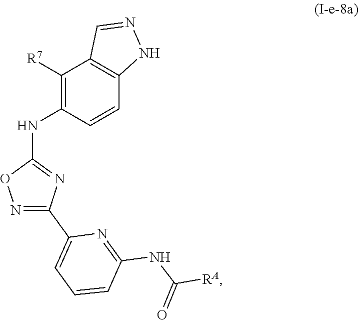
or a pharmaceutically acceptable salt, co-crystal, tautomer, stereoisomer, solvate, hydrate, polymorph, isotopically enriched compound, or prodrug thereof.

120. The compound of any of claims 1-53, wherein the compound is of Formula (I-f): 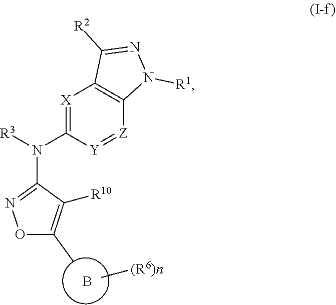
or a pharmaceutically acceptable salt, co-crystal, tautomer, stereoisomer, solvate, hydrate, polymorph, isotopically enriched compound, or prodrug thereof.

121. The compound of any of claims 1-53, wherein the compound is of Formula (I-f-1): 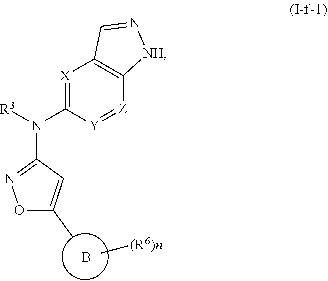
or a pharmaceutically acceptable salt, co-crystal, tautomer, stereoisomer, solvate, hydrate, polymorph, isotopically enriched compound, or prodrug thereof.

122. The compound of any of claims 1-53, wherein the compound is of Formula (I-f-2): 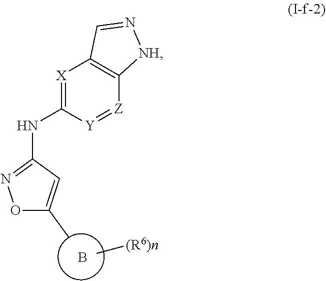
or a pharmaceutically acceptable salt, co-crystal, tautomer, stereoisomer, solvate, hydrate, polymorph, isotopically enriched compound, or prodrug thereof.

123. The compound of any of claims 1-53, wherein the compound is of Formula (I-f-3): 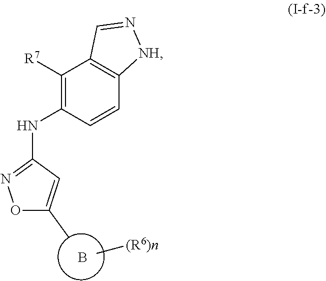
or a pharmaceutically acceptable salt, co-crystal, tautomer, stereoisomer, solvate, hydrate, polymorph, isotopically enriched compound, or prodrug thereof.

124. The compound of any of claims 1-53, wherein the compound is of Formula (I-f-4): 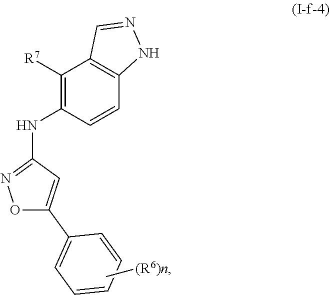
or a pharmaceutically acceptable salt, co-crystal, tautomer, stereoisomer, solvate, hydrate, polymorph, isotopically enriched compound, or prodrug thereof.

125. The compound of any of claims 1-53, wherein the compound is of Formula (I-f-4a): 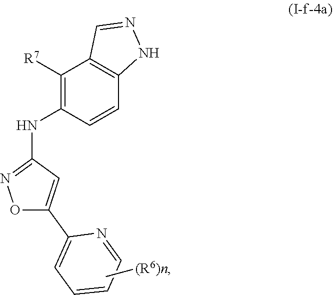
or a pharmaceutically acceptable salt, co-crystal, tautomer, stereoisomer, solvate, hydrate, polymorph, isotopically enriched compound, or prodrug thereof.

126. The compound of any of claims 1-53, wherein the compound is of Formula (I-f-5): 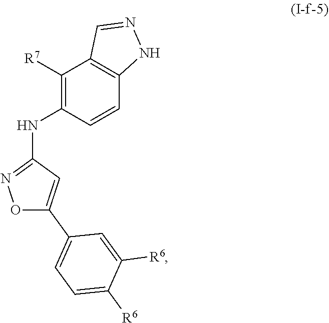
or a pharmaceutically acceptable salt, co-crystal, tautomer, stereoisomer, solvate, hydrate, polymorph, isotopically enriched compound, or prodrug thereof.

127. The compound of any of claims 1-53, wherein the compound is of Formula (I-f-6): 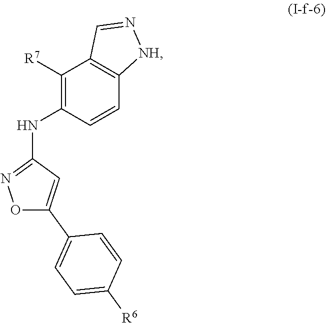
or a pharmaceutically acceptable salt, co-crystal, tautomer, stereoisomer, solvate, hydrate, polymorph, isotopically enriched compound, or prodrug thereof.

128. The compound of any of claims 1-53, wherein the compound is of Formula (I-f-7): 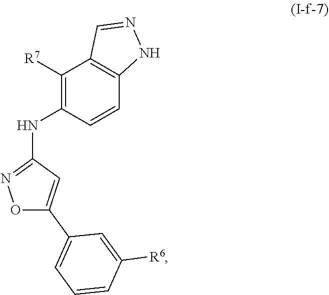
or a pharmaceutically acceptable salt, co-crystal, tautomer, stereoisomer, solvate, hydrate, polymorph, isotopically enriched compound, or prodrug thereof.

129. The compound of any of claims 1-53, wherein the compound is of Formula (I-f-7a): 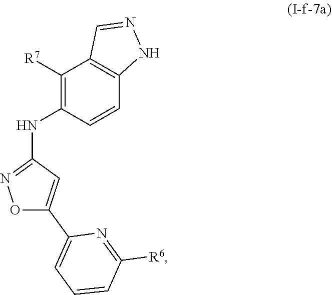
or a pharmaceutically acceptable salt, co-crystal, tautomer, stereoisomer, solvate, hydrate, polymorph, isotopically enriched compound, or prodrug thereof.

130. The compound of any of claims 1-53, wherein the compound is of Formula (I-f-8): 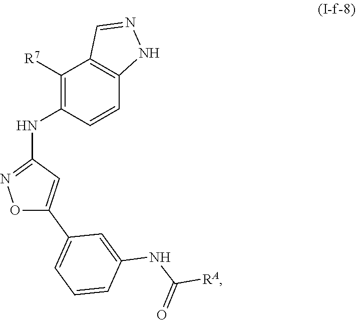
or a pharmaceutically acceptable salt, co-crystal, tautomer, stereoisomer, solvate, hydrate, polymorph, isotopically enriched compound, or prodrug thereof.

131. The compound of any of claims 1-53, wherein the compound is of Formula (I-f-8a): 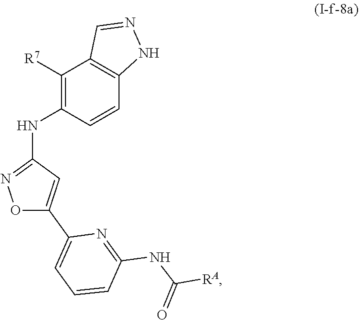
or a pharmaceutically acceptable salt, co-crystal, tautomer, stereoisomer, solvate, hydrate, polymorph, isotopically enriched compound, or prodrug thereof.

132. The compound of claim 1, wherein the compound is: 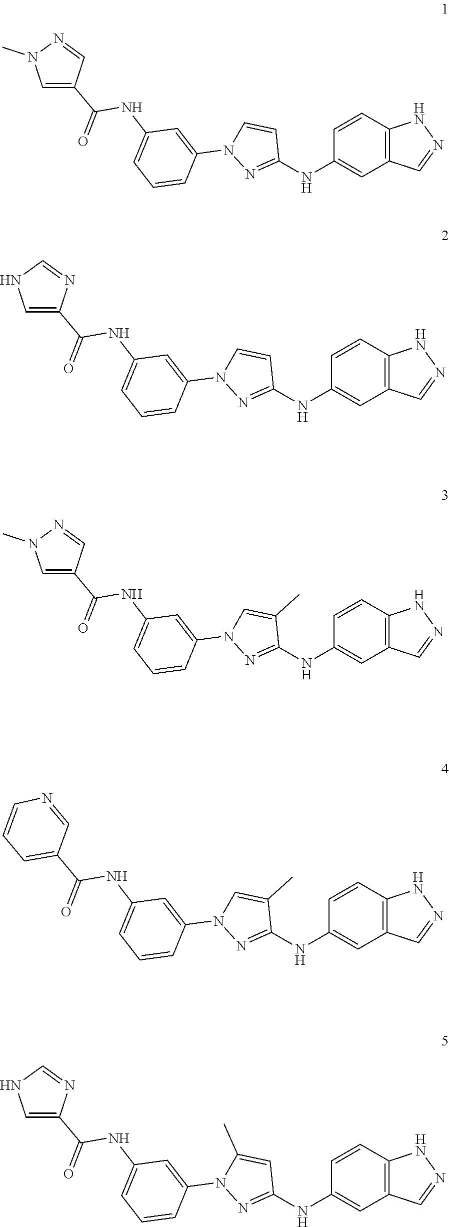 [SMILES: Cn1cc(C(=O)Nc2cccc(-n3ccc(Nc4ccc5[nH]ncc5c4)n3)c2)cn1 ; O=C(Nc1cccc(-n2ccc(Nc3ccc4[nH]ncc4c3)n2)c1)c1c[nH]cn1 ; Cc1cn(-c2cccc(NC(=O)c3cnn(C)c3)c2)nc1Nc1ccc2[nH]ncc2c1 ; Cc1cn(-c2cccc(NC(=O)c3cccnc3)c2)nc1Nc1ccc2[nH]ncc2c1 ; Cc1cc(Nc2ccc3[nH]ncc3c2)nn1-c1cccc(NC(=O)c2c[nH]cn2)c1] 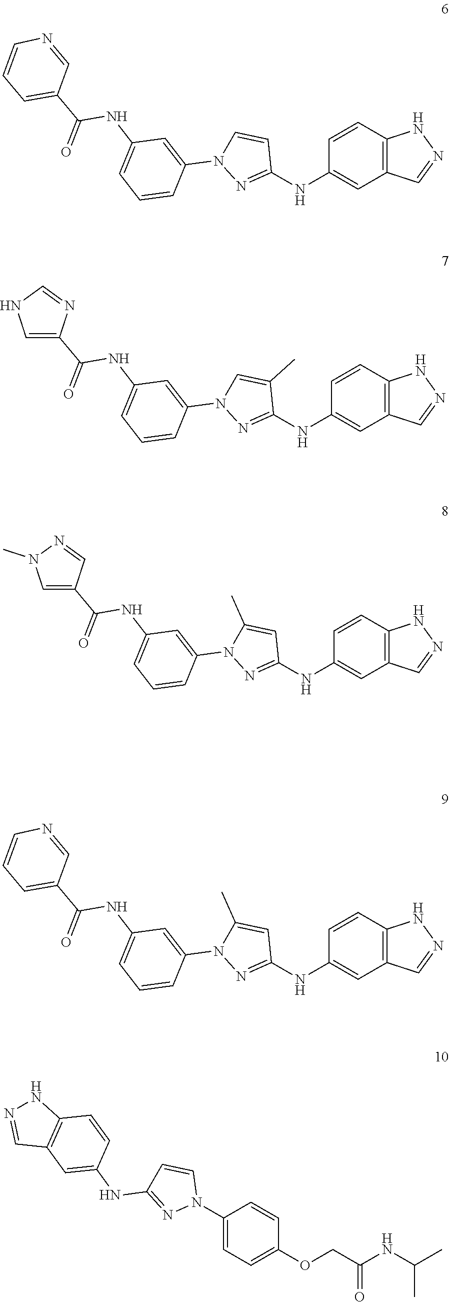 [SMILES: O=C(Nc1cccc(-n2ccc(Nc3ccc4[nH]ncc4c3)n2)c1)c1cccnc1 ; Cc1cn(-c2cccc(NC(=O)c3c[nH]cn3)c2)nc1Nc1ccc2[nH]ncc2c1 ; Cc1cc(Nc2ccc3[nH]ncc3c2)nn1-c1cccc(NC(=O)c2cnn(C)c2)c1 ; Cc1cc(Nc2ccc3[nH]ncc3c2)nn1-c1cccc(NC(=O)c2cccnc2)c1 ; CC(C)NC(=O)COc1ccc(-n2ccc(Nc3ccc4[nH]ncc4c3)n2)cc1] 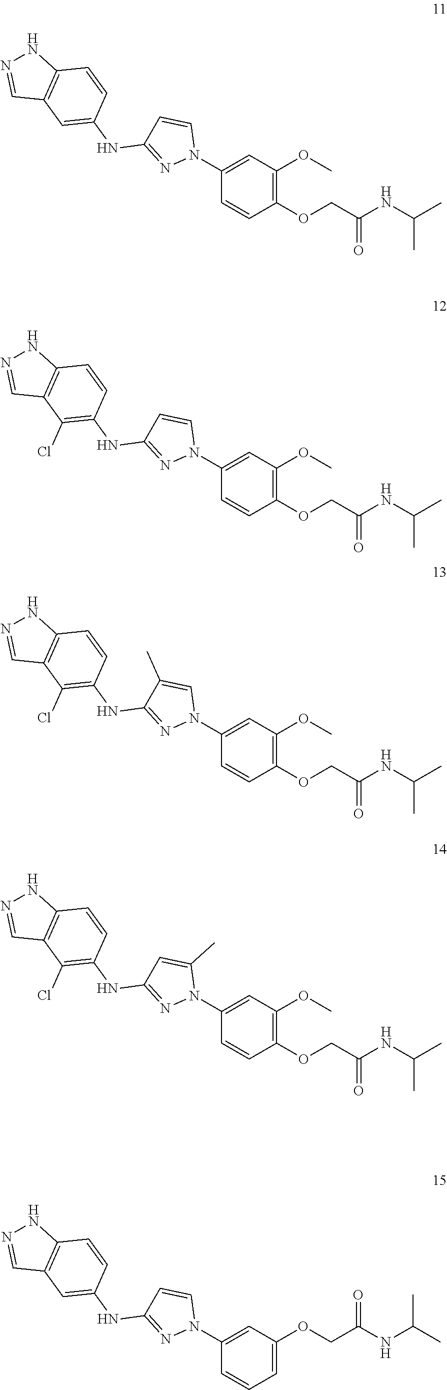 [SMILES: COc1cc(-n2ccc(Nc3ccc4[nH]ncc4c3)n2)ccc1OCC(=O)NC(C)C ; COc1cc(-n2ccc(Nc3ccc4[nH]ncc4c3Cl)n2)ccc1OCC(=O)NC(C)C ; COc1cc(-n2cc(C)c(Nc3ccc4[nH]ncc4c3Cl)n2)ccc1OCC(=O)NC(C)C ; COc1cc(-n2nc(Nc3ccc4[nH]ncc4c3Cl)cc2C)ccc1OCC(=O)NC(C)C ; CC(C)NC(=O)COc1cccc(-n2ccc(Nc3ccc4[nH]ncc4c3)n2)c1] 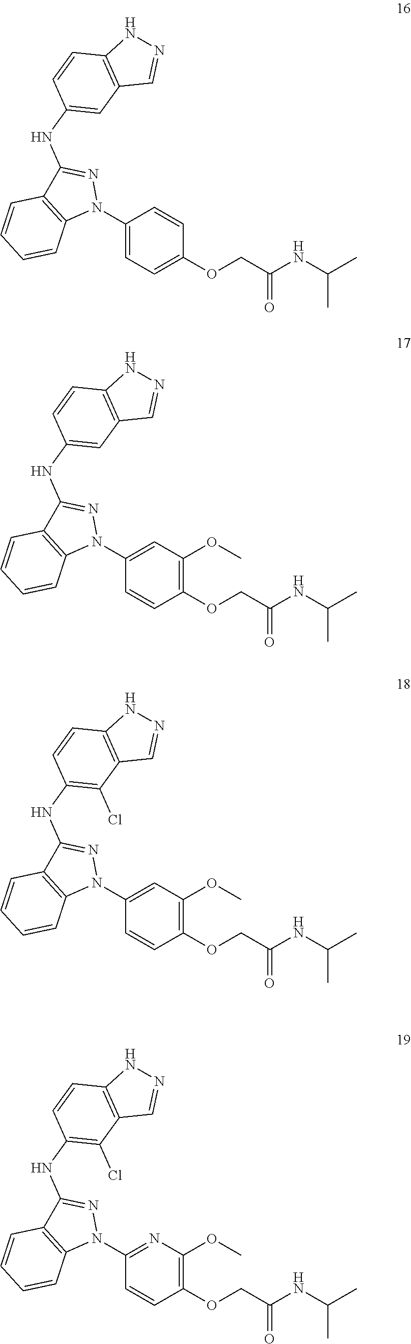 [SMILES: CC(C)NC(=O)COc1ccc(-n2nc(Nc3ccc4[nH]ncc4c3)c3ccccc32)cc1 ; COc1cc(-n2nc(Nc3ccc4[nH]ncc4c3)c3ccccc32)ccc1OCC(=O)NC(C)C ; COc1cc(-n2nc(Nc3ccc4[nH]ncc4c3Cl)c3ccccc32)ccc1OCC(=O)NC(C)C ; COc1nc(-n2nc(Nc3ccc4[nH]ncc4c3Cl)c3ccccc32)ccc1OCC(=O)NC(C)C] 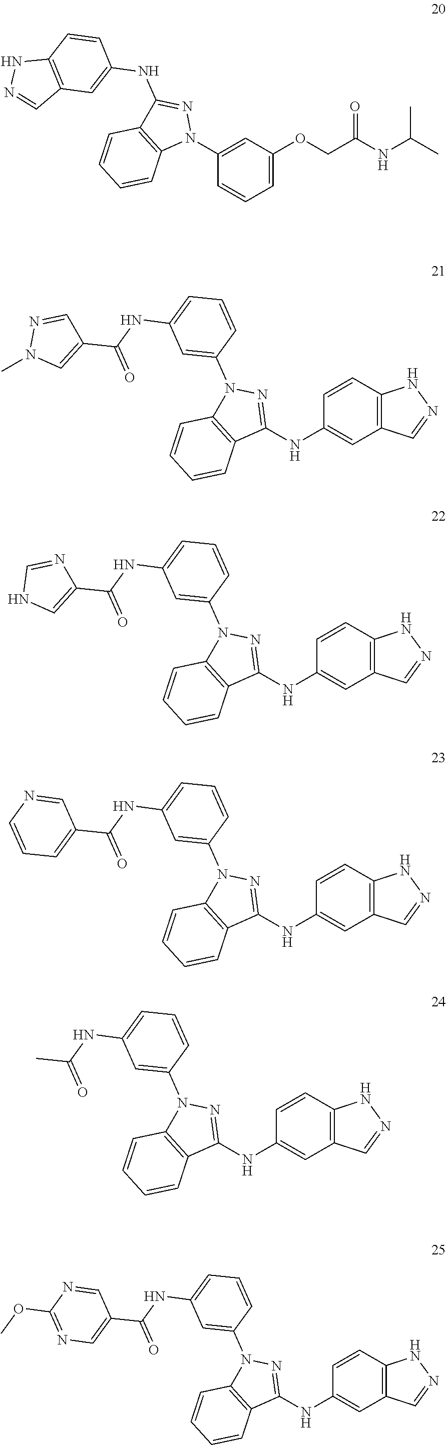 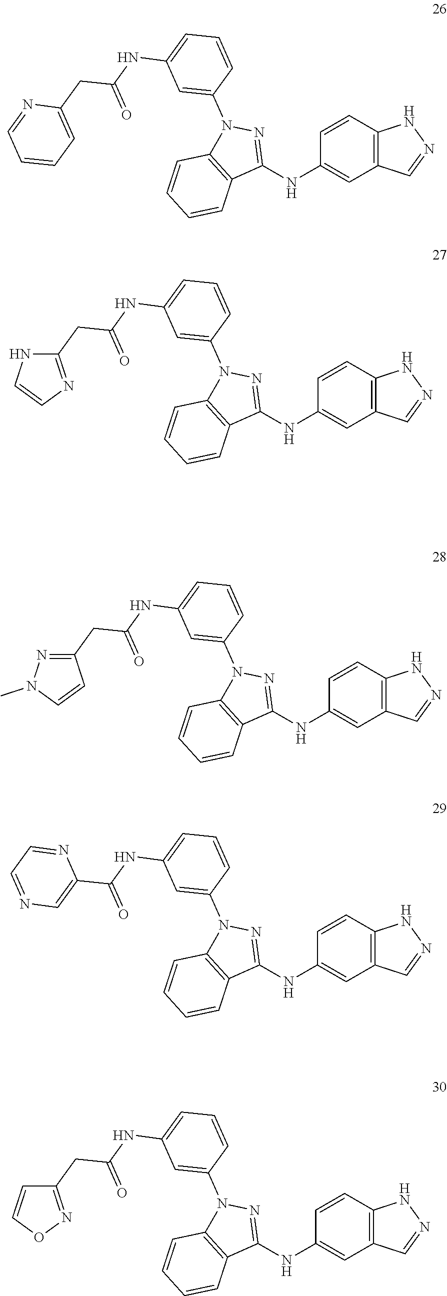 [SMILES: O=C(Cc1ccccn1)Nc1cccc(-n2nc(Nc3ccc4[nH]ncc4c3)c3ccccc32)c1 ; O=C(Cc1ncc[nH]1)Nc1cccc(-n2nc(Nc3ccc4[nH]ncc4c3)c3ccccc32)c1 ; Cn1ccc(CC(=O)Nc2cccc(-n3nc(Nc4ccc5[nH]ncc5c4)c4ccccc43)c2)n1 ; O=C(Nc1cccc(-n2nc(Nc3ccc4[nH]ncc4c3)c3ccccc32)c1)c1cnccn1 ; O=C(Cc1ccon1)Nc1cccc(-n2nc(Nc3ccc4[nH]ncc4c3)c3ccccc32)c1] 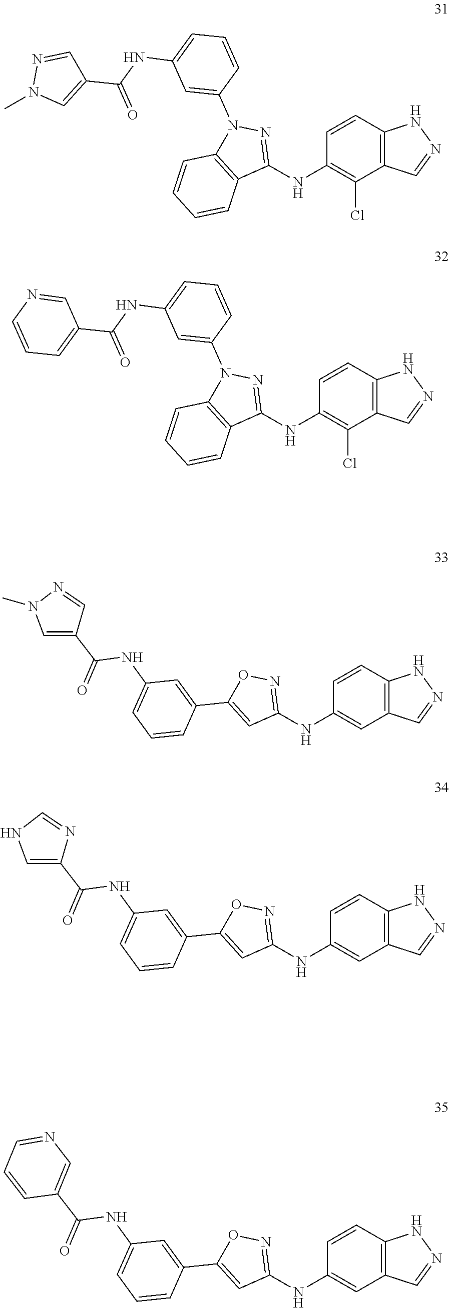 [SMILES: Cn1cc(C(=O)Nc2cccc(-n3nc(Nc4ccc5[nH]ncc5c4Cl)c4ccccc43)c2)cn1 ; O=C(Nc1cccc(-n2nc(Nc3ccc4[nH]ncc4c3Cl)c3ccccc32)c1)c1cccnc1 ; Cn1cc(C(=O)Nc2cccc(-c3cc(Nc4ccc5[nH]ncc5c4)no3)c2)cn1 ; O=C(Nc1cccc(-c2cc(Nc3ccc4[nH]ncc4c3)no2)c1)c1c[nH]cn1 ; O=C(Nc1cccc(-c2cc(Nc3ccc4[nH]ncc4c3)no2)c1)c1cccnc1] 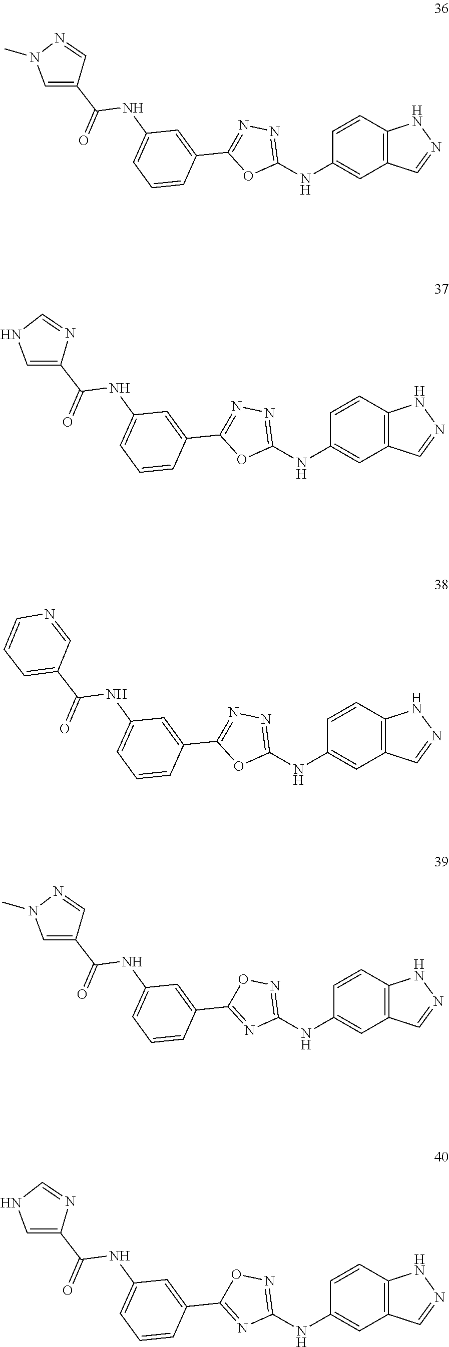 [SMILES: Cn1cc(C(=O)Nc2cccc(-c3nnc(Nc4ccc5[nH]ncc5c4)o3)c2)cn1 ; O=C(Nc1cccc(-c2nnc(Nc3ccc4[nH]ncc4c3)o2)c1)c1c[nH]cn1 ; O=C(Nc1cccc(-c2nnc(Nc3ccc4[nH]ncc4c3)o2)c1)c1cccnc1 ; Cn1cc(C(=O)Nc2cccc(-c3nc(Nc4ccc5[nH]ncc5c4)no3)c2)cn1 ; O=C(Nc1cccc(-c2nc(Nc3ccc4[nH]ncc4c3)no2)c1)c1c[nH]cn1] 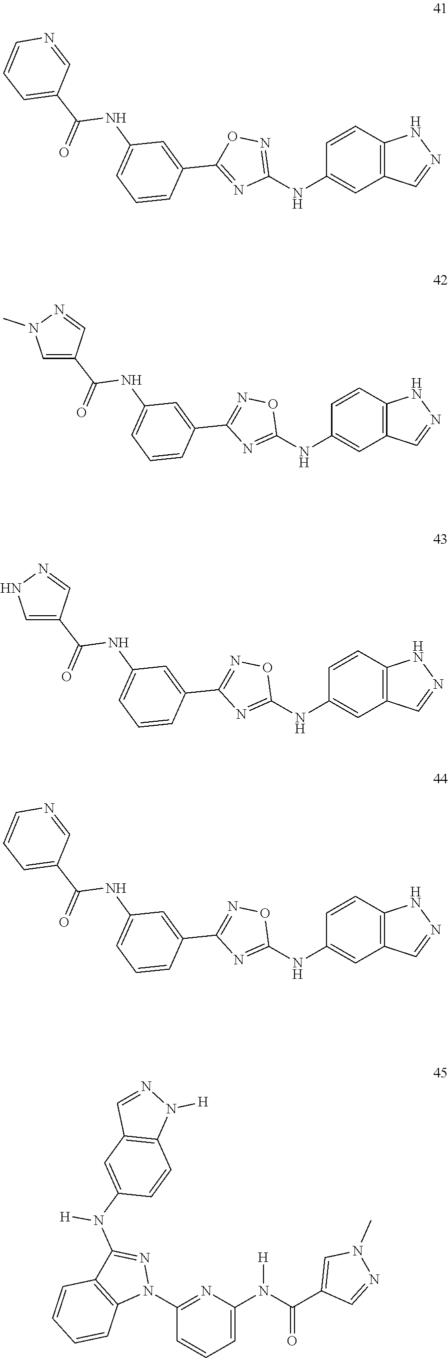 [SMILES: O=C(Nc1cccc(-c2nc(Nc3ccc4[nH]ncc4c3)no2)c1)c1cccnc1 ; Cn1cc(C(=O)Nc2cccc(-c3noc(Nc4ccc5[nH]ncc5c4)n3)c2)cn1 ; O=C(Nc1cccc(-c2noc(Nc3ccc4[nH]ncc4c3)n2)c1)c1cn[nH]c1 ; O=C(Nc1cccc(-c2noc(Nc3ccc4[nH]ncc4c3)n2)c1)c1cccnc1 ; Cn1cc(C(=O)Nc2cccc(-n3nc(Nc4ccc5[nH]ncc5c4)c4ccccc43)n2)cn1] 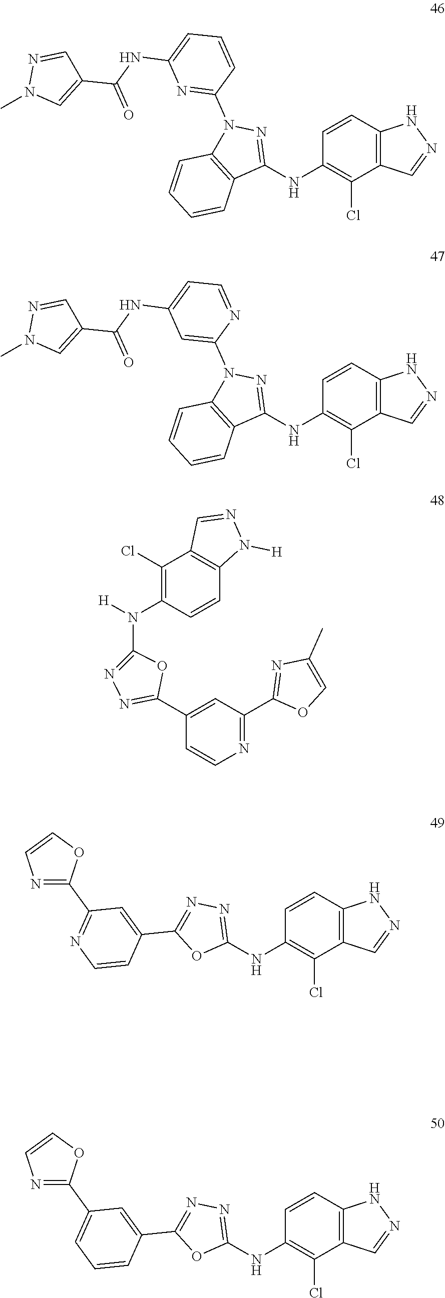 [SMILES: Cn1cc(C(=O)Nc2cccc(-n3nc(Nc4ccc5[nH]ncc5c4Cl)c4ccccc43)n2)cn1 ; Cn1cc(C(=O)Nc2ccnc(-n3nc(Nc4ccc5[nH]ncc5c4Cl)c4ccccc43)c2)cn1 ; Cc1coc(-c2cc(-c3nnc(Nc4ccc5[nH]ncc5c4Cl)o3)ccn2)n1 ; Clc1c(Nc2nnc(-c3ccnc(-c4ncco4)c3)o2)ccc2[nH]ncc12 ; Clc1c(Nc2nnc(-c3cccc(-c4ncco4)c3)o2)ccc2[nH]ncc12] 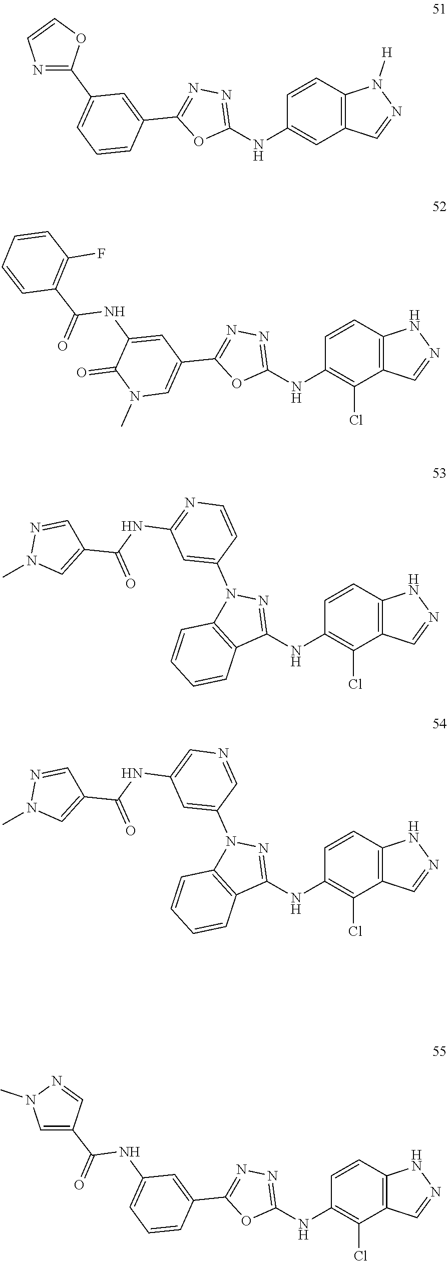 [SMILES: c1cc(-c2ncco2)cc(-c2nnc(Nc3ccc4[nH]ncc4c3)o2)c1 ; Cn1cc(-c2nnc(Nc3ccc4[nH]ncc4c3Cl)o2)cc(NC(=O)c2ccccc2F)c1=O ; Cn1cc(C(=O)Nc2cc(-n3nc(Nc4ccc5[nH]ncc5c4Cl)c4ccccc43)ccn2)cn1 ; Cn1cc(C(=O)Nc2cncc(-n3nc(Nc4ccc5[nH]ncc5c4Cl)c4ccccc43)c2)cn1 ; Cn1cc(C(=O)Nc2cccc(-c3nnc(Nc4ccc5[nH]ncc5c4Cl)o3)c2)cn1] 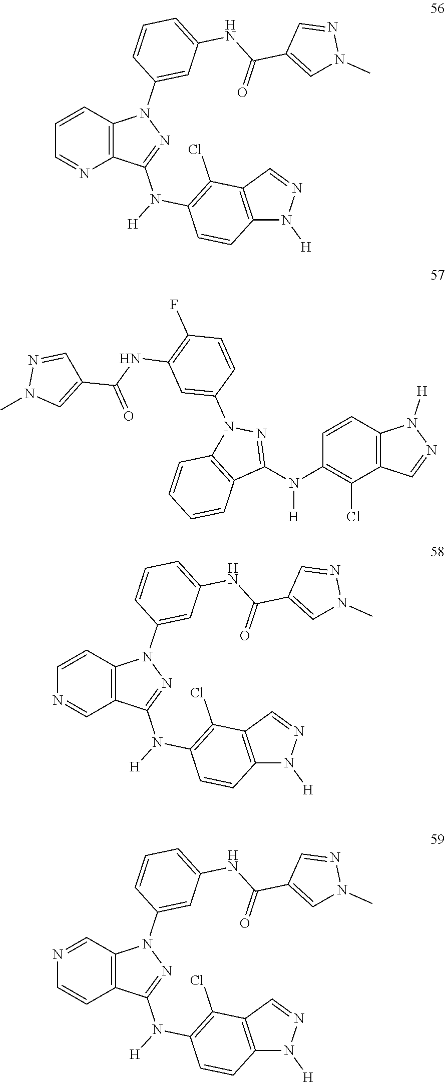 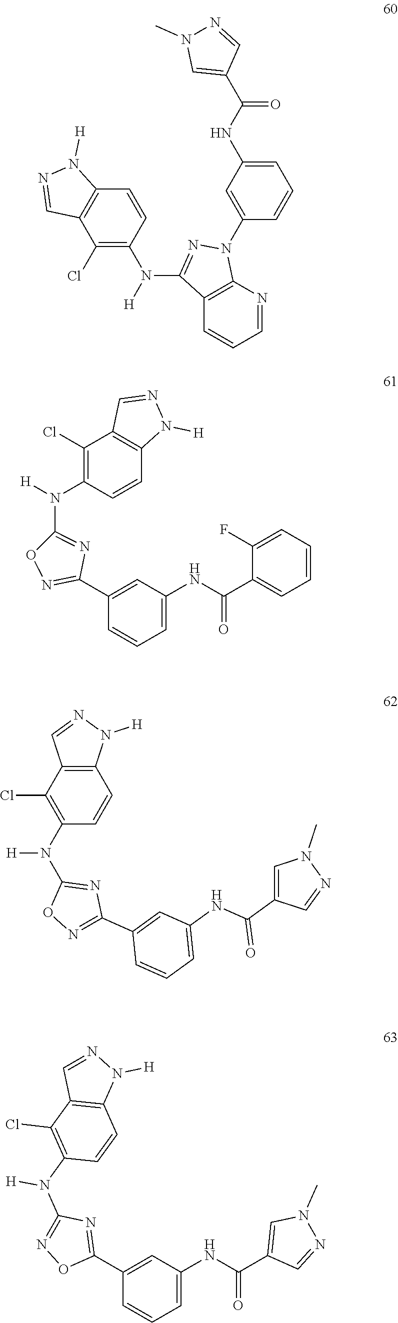 [SMILES: Cn1cc(C(=O)Nc2cccc(-n3nc(Nc4ccc5[nH]ncc5c4Cl)c4cccnc43)c2)cn1 ; O=C(Nc1cccc(-c2noc(Nc3ccc4[nH]ncc4c3Cl)n2)c1)c1ccccc1F ; Cn1cc(C(=O)Nc2cccc(-c3noc(Nc4ccc5[nH]ncc5c4Cl)n3)c2)cn1 ; Cn1cc(C(=O)Nc2cccc(-c3nc(Nc4ccc5[nH]ncc5c4Cl)no3)c2)cn1] 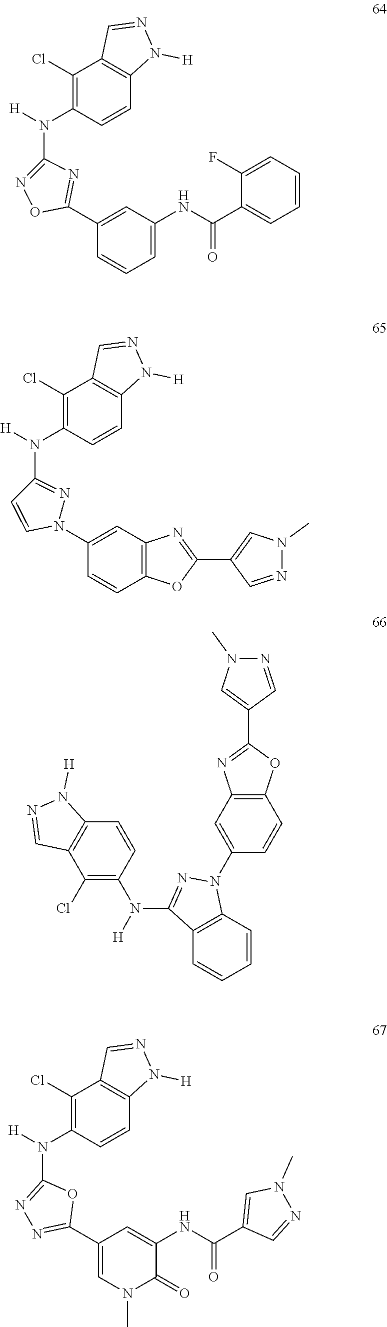 [SMILES: O=C(Nc1cccc(-c2nc(Nc3ccc4[nH]ncc4c3Cl)no2)c1)c1ccccc1F ; Cn1cc(-c2nc3cc(-n4ccc(Nc5ccc6[nH]ncc6c5Cl)n4)ccc3o2)cn1 ; Cn1cc(-c2nc3cc(-n4nc(Nc5ccc6[nH]ncc6c5Cl)c5ccccc54)ccc3o2)cn1 ; Cn1cc(C(=O)Nc2cc(-c3nnc(Nc4ccc5[nH]ncc5c4Cl)o3)cn(C)c2=O)cn1] 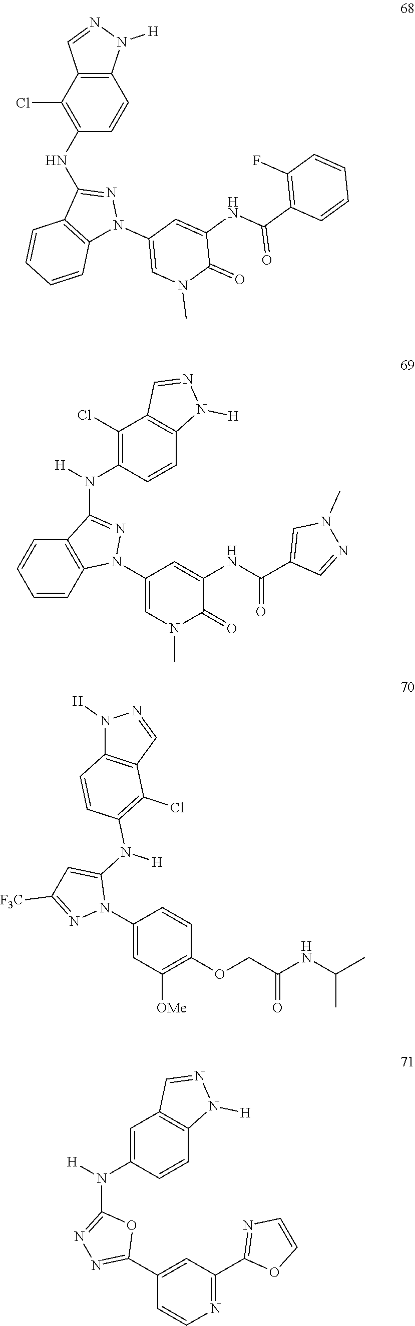 [SMILES: Cn1cc(-n2nc(Nc3ccc4[nH]ncc4c3Cl)c3ccccc32)cc(NC(=O)c2ccccc2F)c1=O ; Cn1cc(C(=O)Nc2cc(-n3nc(Nc4ccc5[nH]ncc5c4Cl)c4ccccc43)cn(C)c2=O)cn1 ; COc1cc(-n2nc(C(F)(F)F)cc2Nc2ccc3[nH]ncc3c2Cl)ccc1OCC(=O)NC(C)C ; c1cc(-c2nnc(Nc3ccc4[nH]ncc4c3)o2)cc(-c2ncco2)n1] 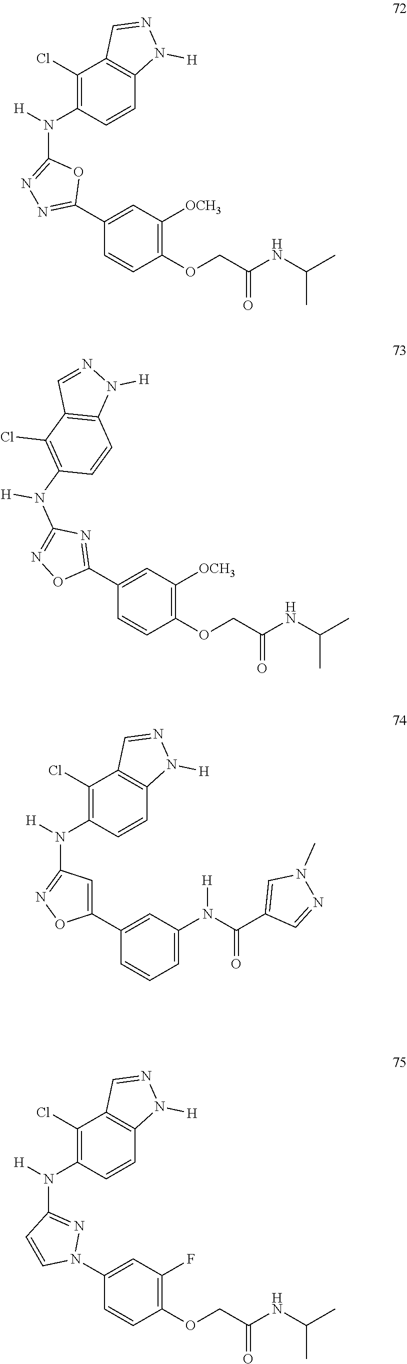 [SMILES: COc1cc(-c2nnc(Nc3ccc4[nH]ncc4c3Cl)o2)ccc1OCC(=O)NC(C)C ; COc1cc(-c2nc(Nc3ccc4[nH]ncc4c3Cl)no2)ccc1OCC(=O)NC(C)C ; Cn1cc(C(=O)Nc2cccc(-c3cc(Nc4ccc5[nH]ncc5c4Cl)no3)c2)cn1 ; CC(C)NC(=O)COc1ccc(-n2ccc(Nc3ccc4[nH]ncc4c3Cl)n2)cc1F] 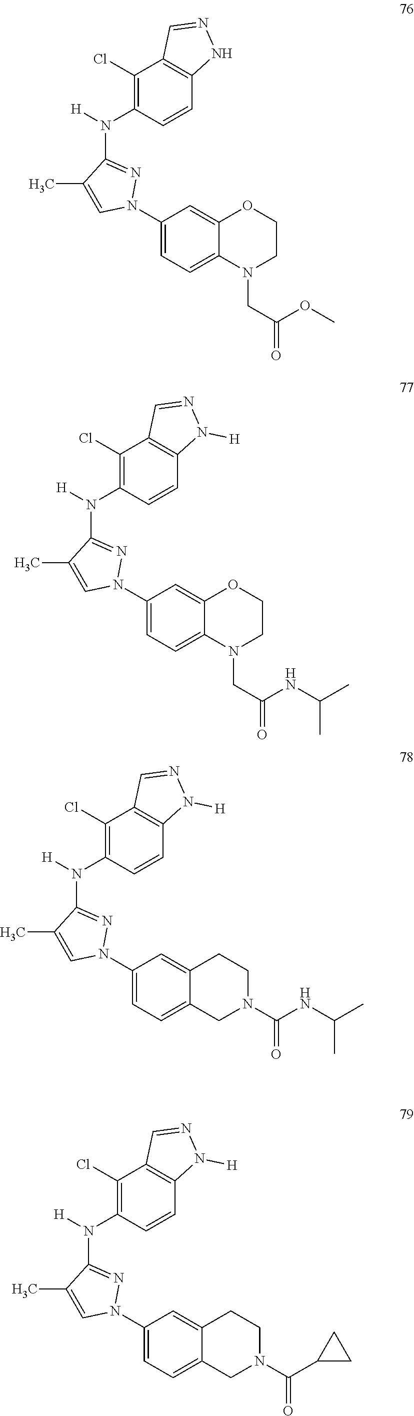 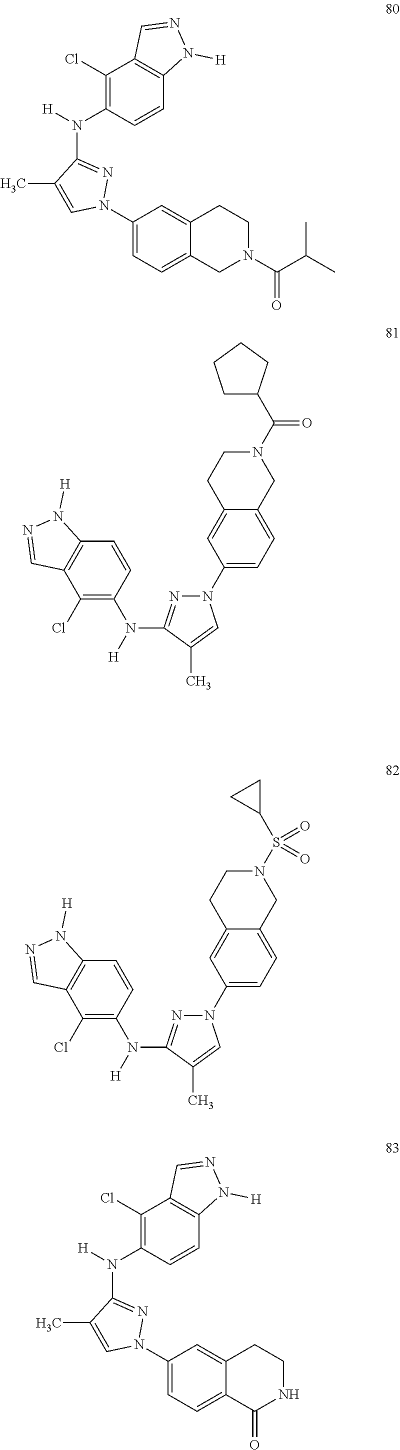 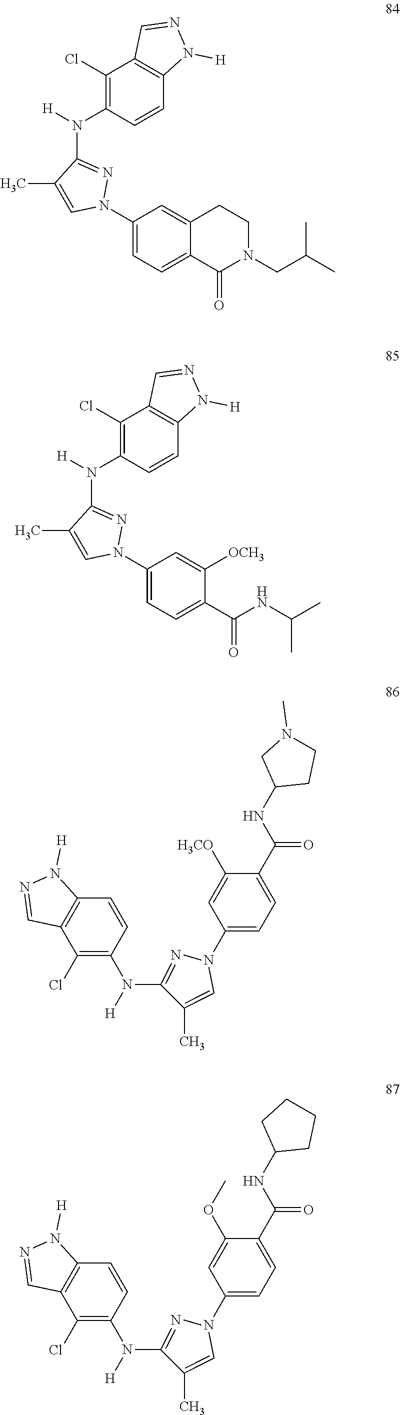 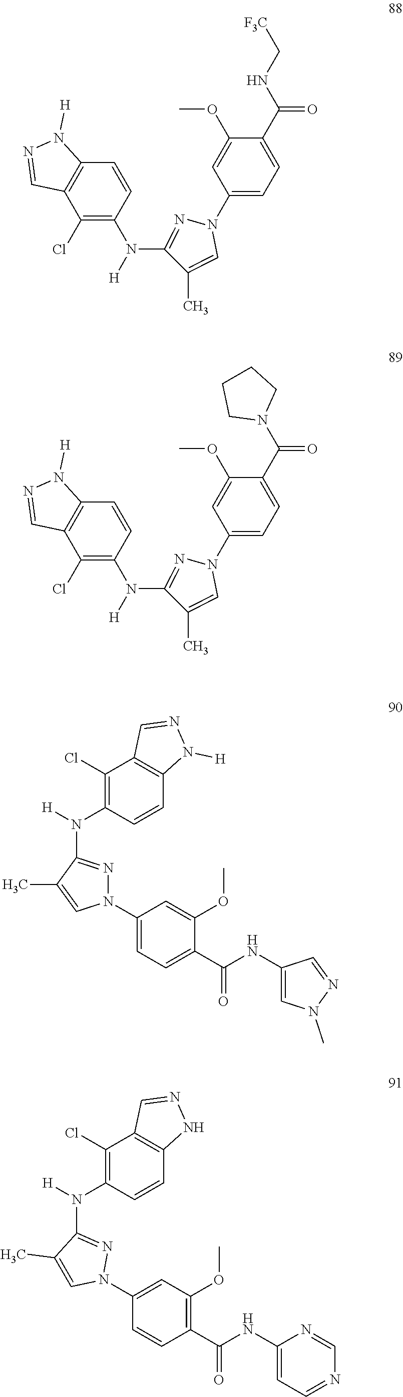 [SMILES: COc1cc(-n2cc(C)c(Nc3ccc4[nH]ncc4c3Cl)n2)ccc1C(=O)NCC(F)(F)F ; COc1cc(-n2cc(C)c(Nc3ccc4[nH]ncc4c3Cl)n2)ccc1C(=O)N1CCCC1 ; COc1cc(-n2cc(C)c(Nc3ccc4[nH]ncc4c3Cl)n2)ccc1C(=O)Nc1cnn(C)c1 ; COc1cc(-n2cc(C)c(Nc3ccc4[nH]ncc4c3Cl)n2)ccc1C(=O)Nc1ccncn1] 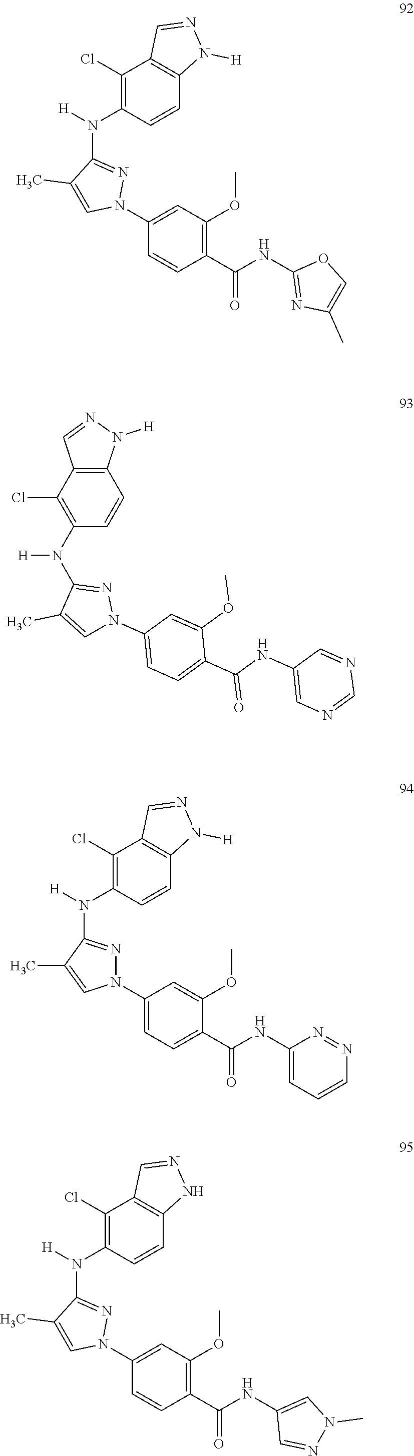 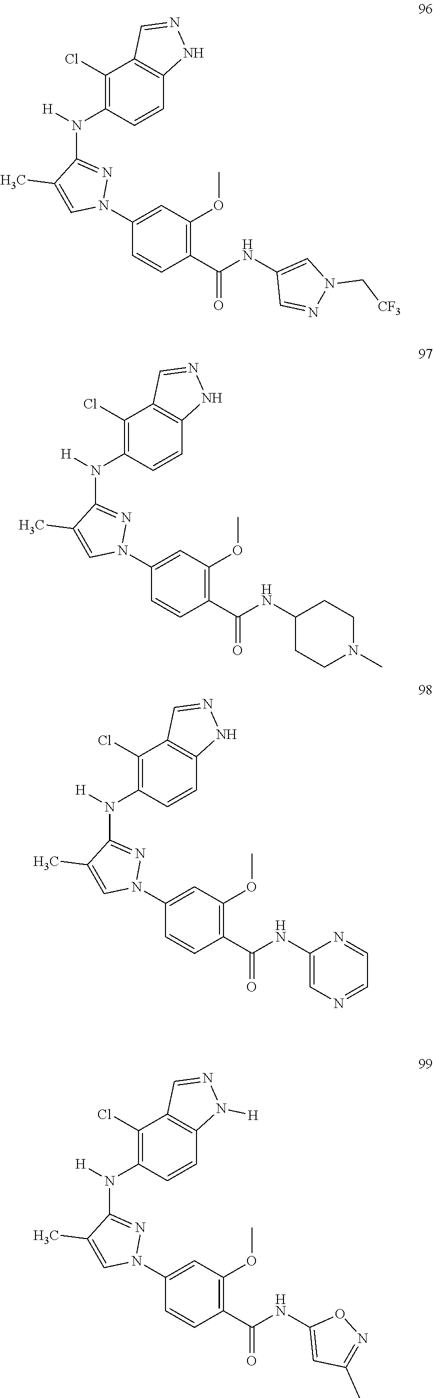 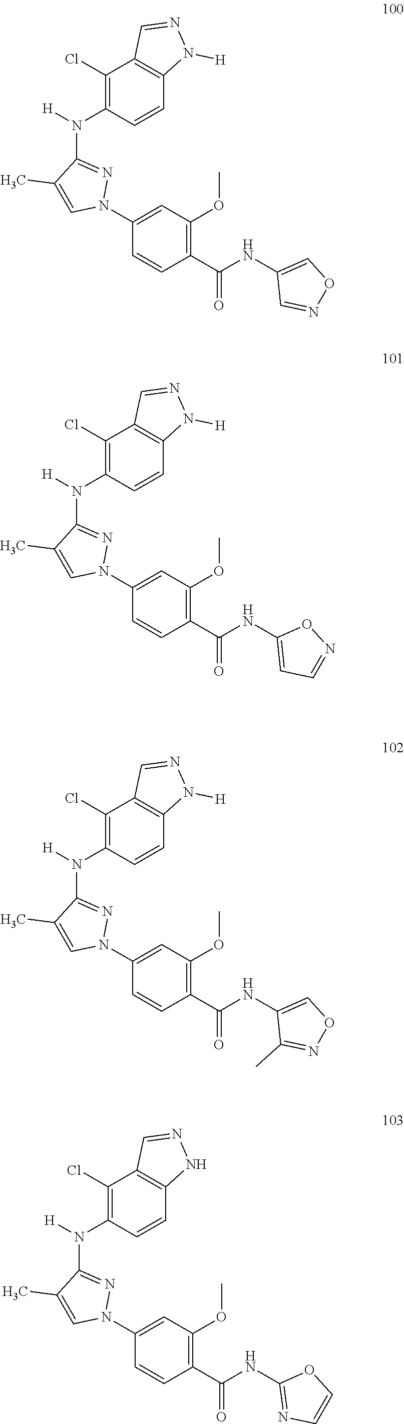 [SMILES: COc1cc(-n2cc(C)c(Nc3ccc4[nH]ncc4c3Cl)n2)ccc1C(=O)Nc1cnoc1 ; COc1cc(-n2cc(C)c(Nc3ccc4[nH]ncc4c3Cl)n2)ccc1C(=O)Nc1ccno1 ; COc1cc(-n2cc(C)c(Nc3ccc4[nH]ncc4c3Cl)n2)ccc1C(=O)Nc1conc1C ; COc1cc(-n2cc(C)c(Nc3ccc4[nH]ncc4c3Cl)n2)ccc1C(=O)Nc1ncco1] 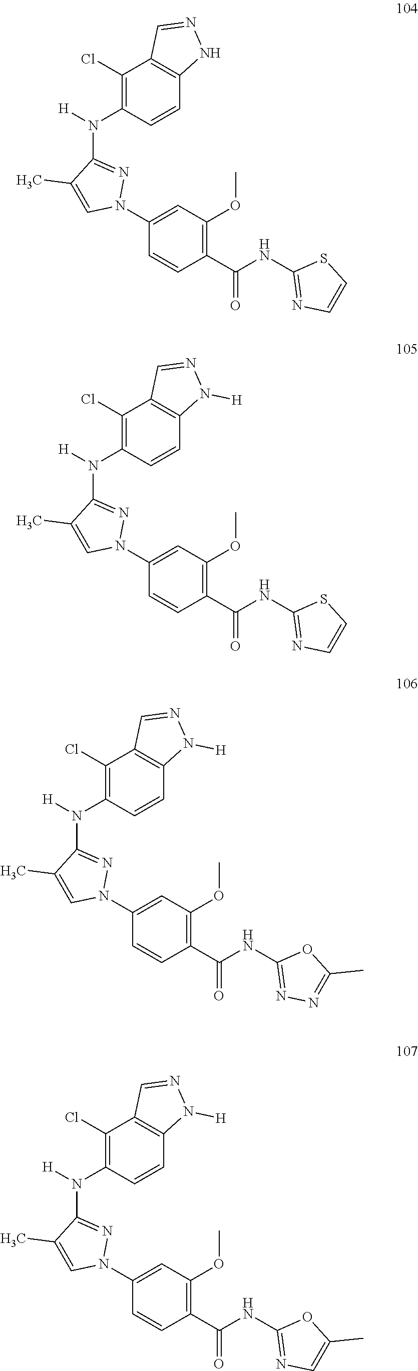 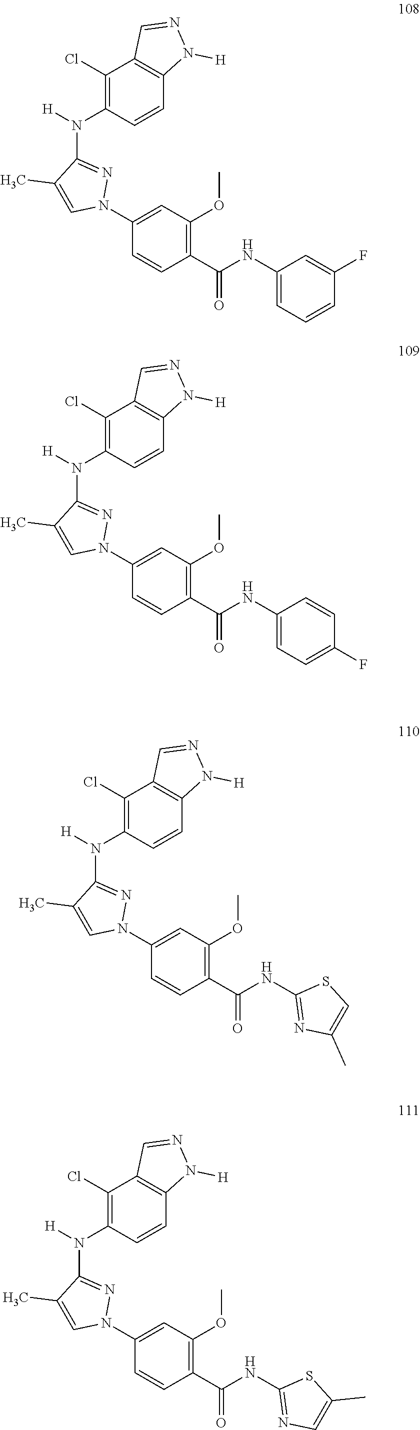 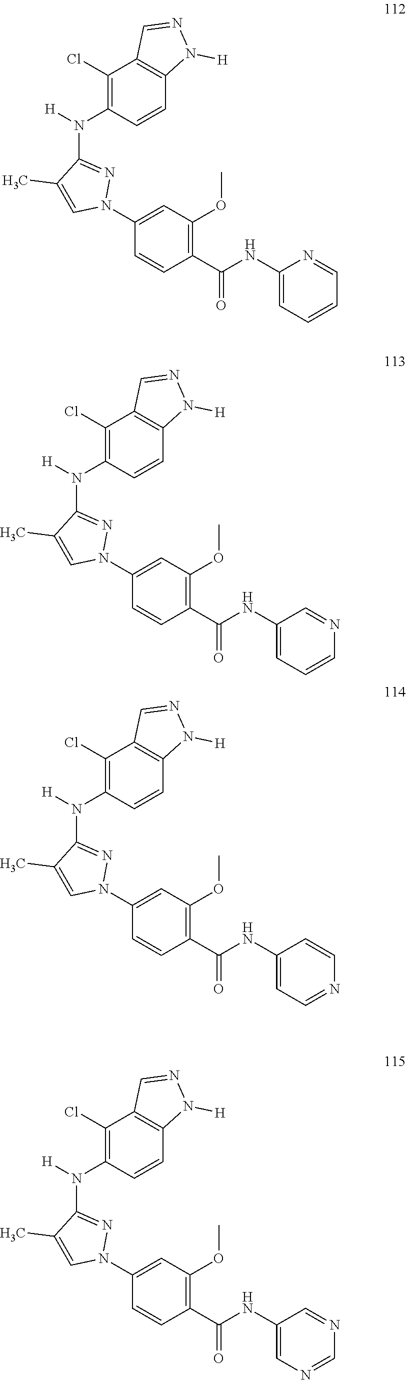 [SMILES: COc1cc(-n2cc(C)c(Nc3ccc4[nH]ncc4c3Cl)n2)ccc1C(=O)Nc1ccccn1 ; COc1cc(-n2cc(C)c(Nc3ccc4[nH]ncc4c3Cl)n2)ccc1C(=O)Nc1cccnc1 ; COc1cc(-n2cc(C)c(Nc3ccc4[nH]ncc4c3Cl)n2)ccc1C(=O)Nc1ccncc1 ; COc1cc(-n2cc(C)c(Nc3ccc4[nH]ncc4c3Cl)n2)ccc1C(=O)Nc1cncnc1] 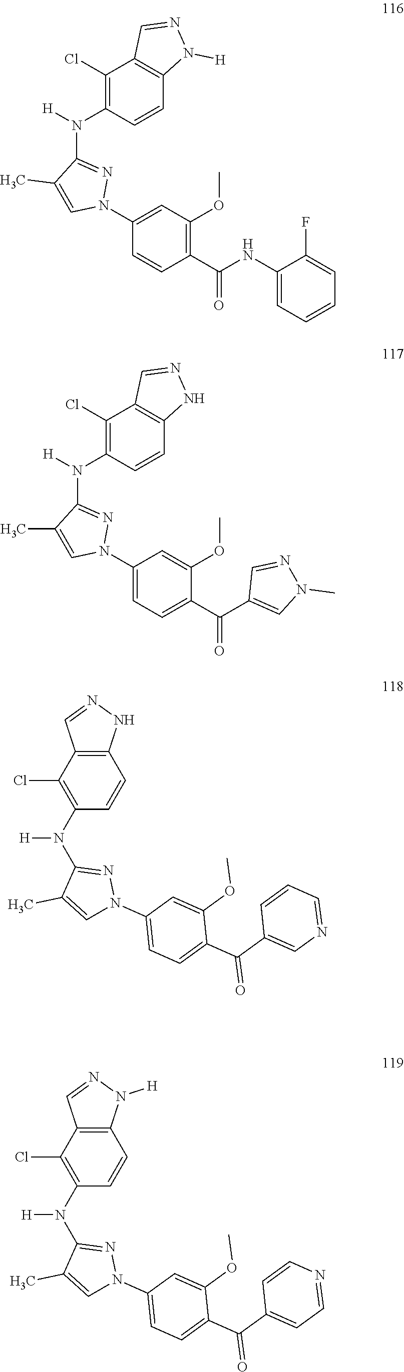 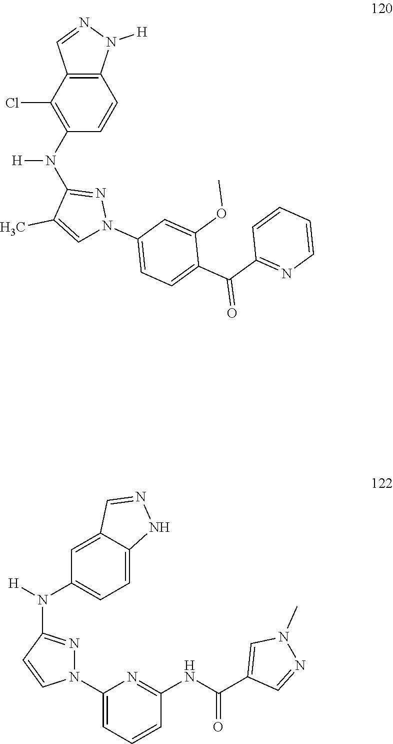 [SMILES: COc1cc(-n2cc(C)c(Nc3ccc4[nH]ncc4c3Cl)n2)ccc1C(=O)c1ccccn1 ; Cn1cc(C(=O)Nc2cccc(-n3ccc(Nc4ccc5[nH]ncc5c4)n3)n2)cn1] 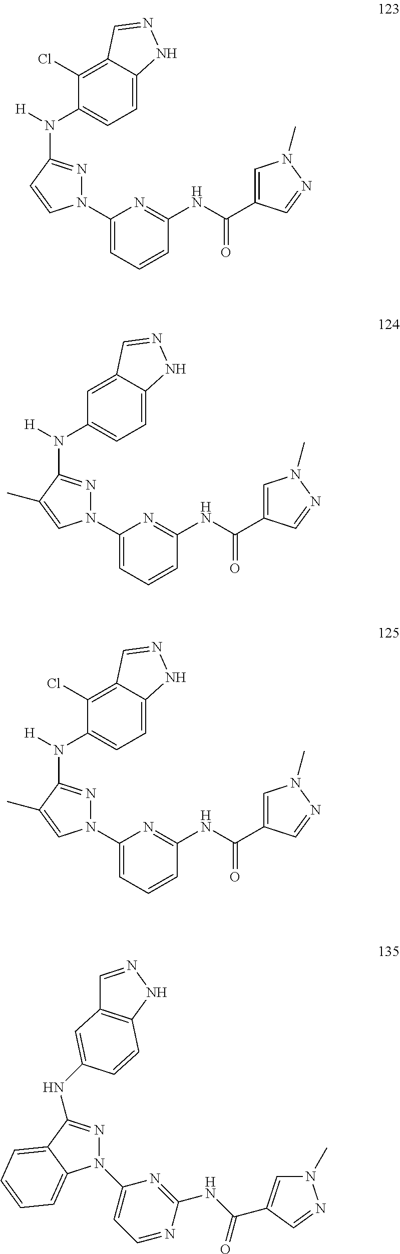 [SMILES: Cn1cc(C(=O)Nc2cccc(-n3ccc(Nc4ccc5[nH]ncc5c4Cl)n3)n2)cn1 ; Cc1cn(-c2cccc(NC(=O)c3cnn(C)c3)n2)nc1Nc1ccc2[nH]ncc2c1 ; Cc1cn(-c2cccc(NC(=O)c3cnn(C)c3)n2)nc1Nc1ccc2[nH]ncc2c1Cl ; Cn1cc(C(=O)Nc2nccc(-n3nc(Nc4ccc5[nH]ncc5c4)c4ccccc43)n2)cn1] 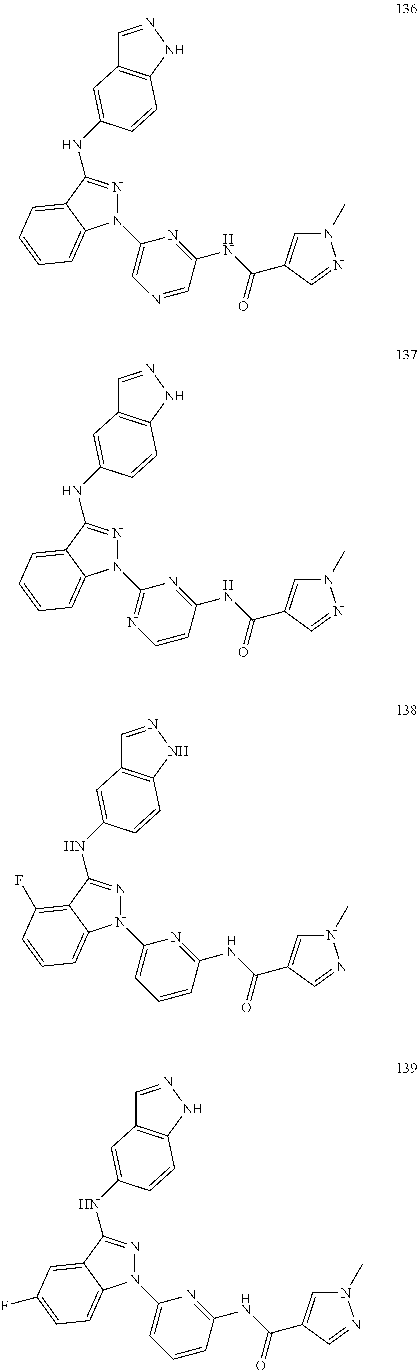 [SMILES: Cn1cc(C(=O)Nc2cncc(-n3nc(Nc4ccc5[nH]ncc5c4)c4ccccc43)n2)cn1 ; Cn1cc(C(=O)Nc2ccnc(-n3nc(Nc4ccc5[nH]ncc5c4)c4ccccc43)n2)cn1 ; Cn1cc(C(=O)Nc2cccc(-n3nc(Nc4ccc5[nH]ncc5c4)c4c(F)cccc43)n2)cn1 ; Cn1cc(C(=O)Nc2cccc(-n3nc(Nc4ccc5[nH]ncc5c4)c4cc(F)ccc43)n2)cn1] 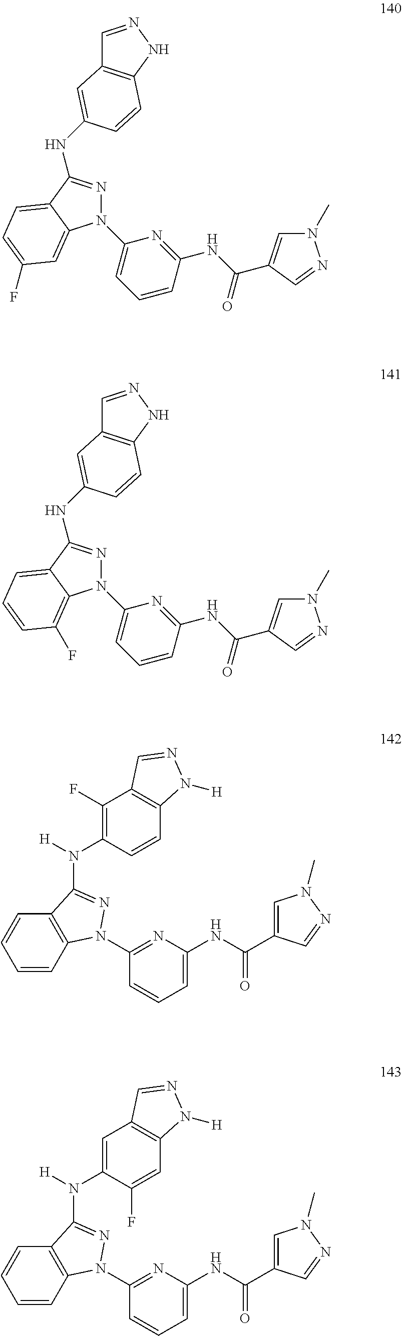 [SMILES: Cn1cc(C(=O)Nc2cccc(-n3nc(Nc4ccc5[nH]ncc5c4)c4ccc(F)cc43)n2)cn1 ; Cn1cc(C(=O)Nc2cccc(-n3nc(Nc4ccc5[nH]ncc5c4)c4cccc(F)c43)n2)cn1 ; Cn1cc(C(=O)Nc2cccc(-n3nc(Nc4ccc5[nH]ncc5c4F)c4ccccc43)n2)cn1 ; Cn1cc(C(=O)Nc2cccc(-n3nc(Nc4cc5cn[nH]c5cc4F)c4ccccc43)n2)cn1] 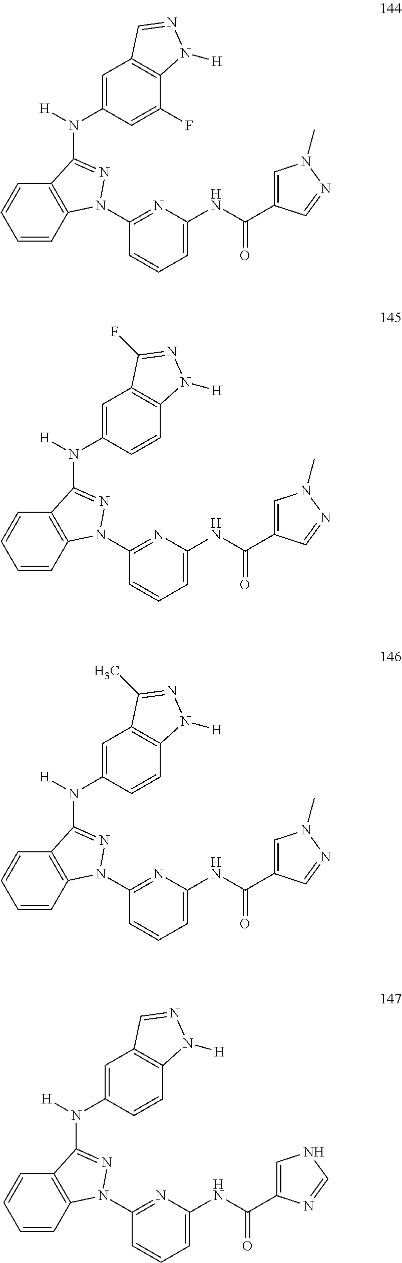 [SMILES: Cn1cc(C(=O)Nc2cccc(-n3nc(Nc4cc(F)c5[nH]ncc5c4)c4ccccc43)n2)cn1 ; Cn1cc(C(=O)Nc2cccc(-n3nc(Nc4ccc5[nH]nc(F)c5c4)c4ccccc43)n2)cn1 ; Cc1n[nH]c2ccc(Nc3nn(-c4cccc(NC(=O)c5cnn(C)c5)n4)c4ccccc34)cc12 ; O=C(Nc1cccc(-n2nc(Nc3ccc4[nH]ncc4c3)c3ccccc32)n1)c1c[nH]cn1] 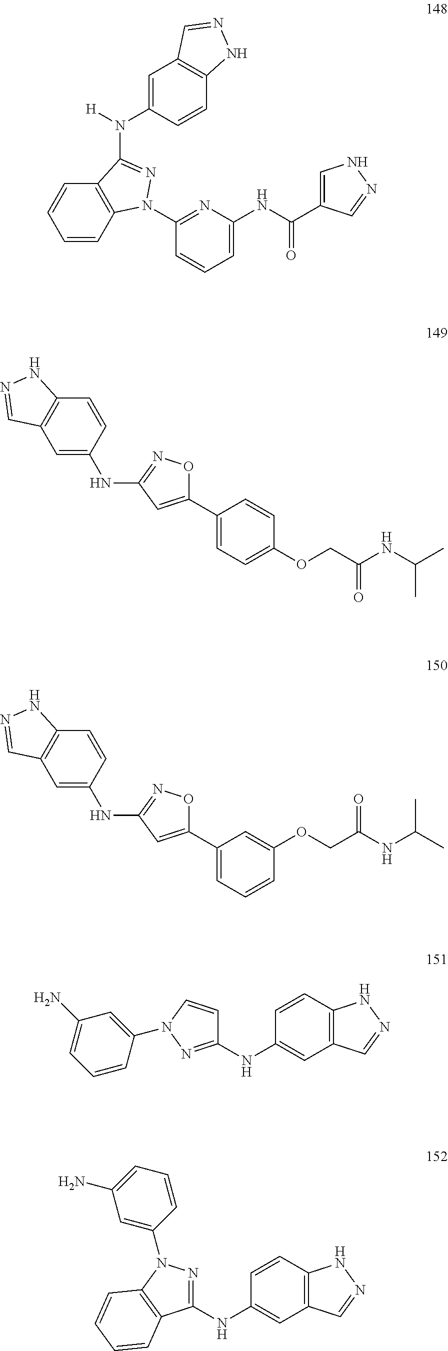 [SMILES: O=C(Nc1cccc(-n2nc(Nc3ccc4[nH]ncc4c3)c3ccccc32)n1)c1cn[nH]c1 ; CC(C)NC(=O)COc1ccc(-c2cc(Nc3ccc4[nH]ncc4c3)no2)cc1 ; CC(C)NC(=O)COc1cccc(-c2cc(Nc3ccc4[nH]ncc4c3)no2)c1 ; Nc1cccc(-n2ccc(Nc3ccc4[nH]ncc4c3)n2)c1 ; Nc1cccc(-n2nc(Nc3ccc4[nH]ncc4c3)c3ccccc32)c1] 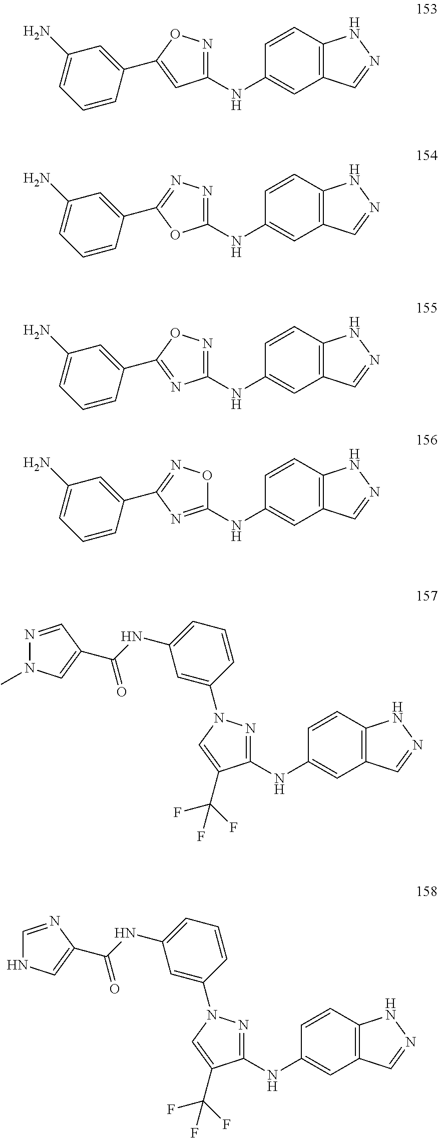 [SMILES: Nc1cccc(-c2cc(Nc3ccc4[nH]ncc4c3)no2)c1 ; Nc1cccc(-c2nnc(Nc3ccc4[nH]ncc4c3)o2)c1 ; Nc1cccc(-c2nc(Nc3ccc4[nH]ncc4c3)no2)c1 ; Nc1cccc(-c2noc(Nc3ccc4[nH]ncc4c3)n2)c1 ; Cn1cc(C(=O)Nc2cccc(-n3cc(C(F)(F)F)c(Nc4ccc5[nH]ncc5c4)n3)c2)cn1 ; O=C(Nc1cccc(-n2cc(C(F)(F)F)c(Nc3ccc4[nH]ncc4c3)n2)c1)c1c[nH]cn1] 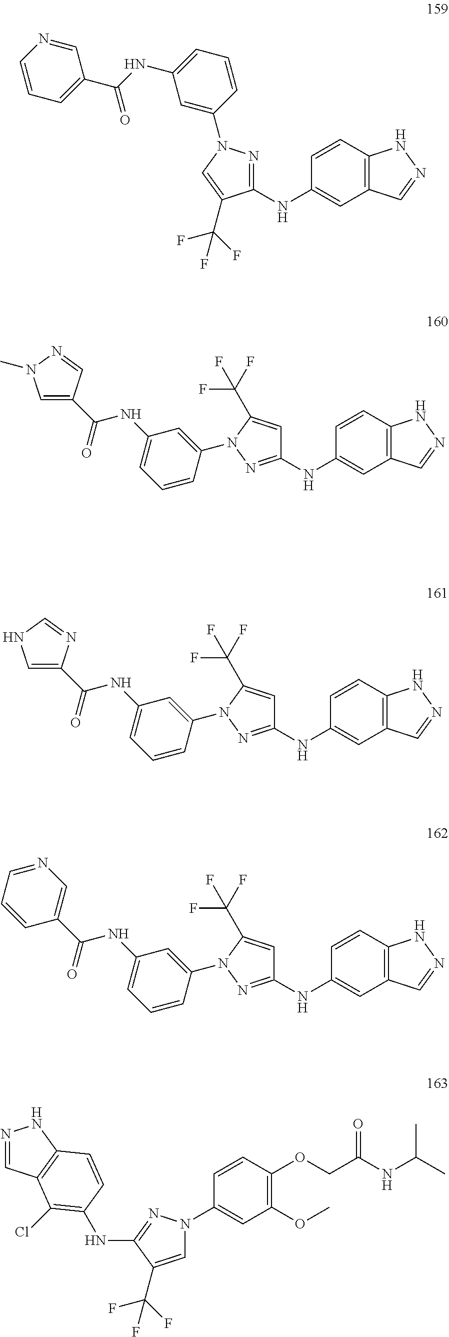 [SMILES: O=C(Nc1cccc(-n2cc(C(F)(F)F)c(Nc3ccc4[nH]ncc4c3)n2)c1)c1cccnc1 ; Cn1cc(C(=O)Nc2cccc(-n3nc(Nc4ccc5[nH]ncc5c4)cc3C(F)(F)F)c2)cn1 ; O=C(Nc1cccc(-n2nc(Nc3ccc4[nH]ncc4c3)cc2C(F)(F)F)c1)c1c[nH]cn1 ; O=C(Nc1cccc(-n2nc(Nc3ccc4[nH]ncc4c3)cc2C(F)(F)F)c1)c1cccnc1 ; COc1cc(-n2cc(C(F)(F)F)c(Nc3ccc4[nH]ncc4c3Cl)n2)ccc1OCC(=O)NC(C)C] 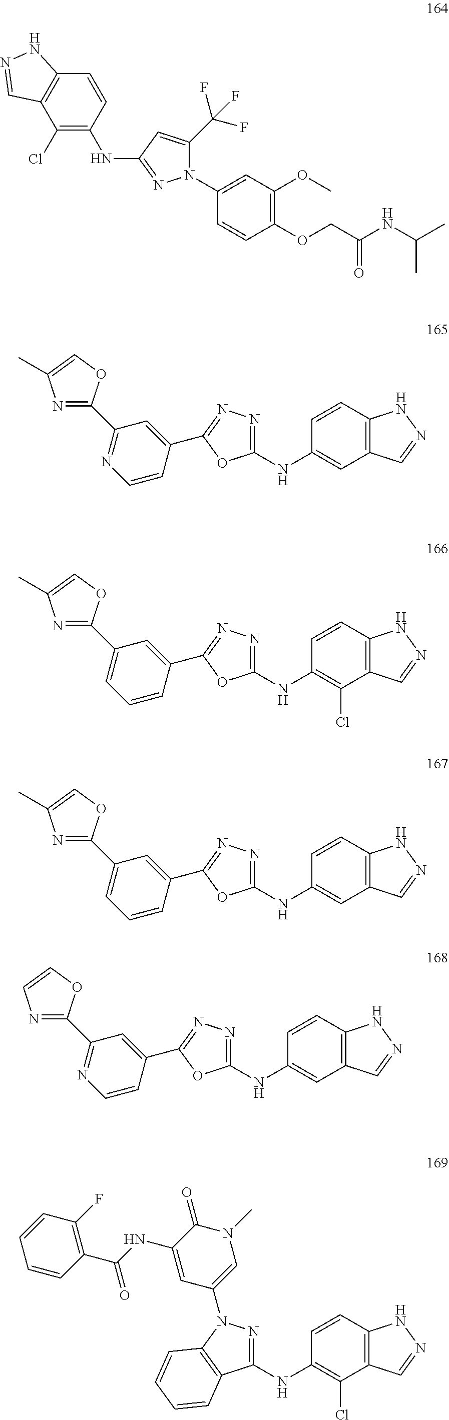 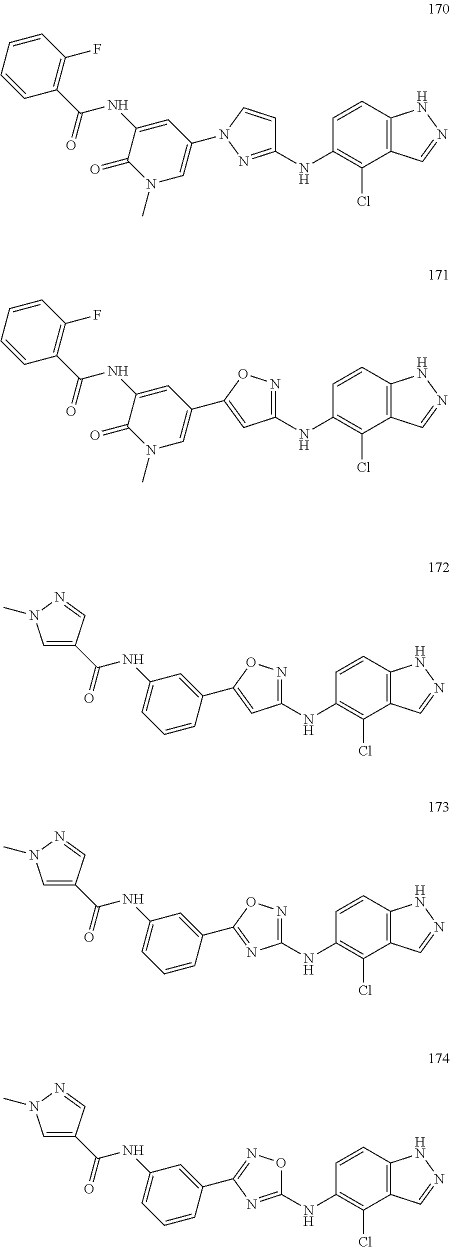 [SMILES: Cn1cc(-n2ccc(Nc3ccc4[nH]ncc4c3Cl)n2)cc(NC(=O)c2ccccc2F)c1=O ; Cn1cc(-c2cc(Nc3ccc4[nH]ncc4c3Cl)no2)cc(NC(=O)c2ccccc2F)c1=O ; Cn1cc(C(=O)Nc2cccc(-c3cc(Nc4ccc5[nH]ncc5c4Cl)no3)c2)cn1 ; Cn1cc(C(=O)Nc2cccc(-c3nc(Nc4ccc5[nH]ncc5c4Cl)no3)c2)cn1 ; Cn1cc(C(=O)Nc2cccc(-c3noc(Nc4ccc5[nH]ncc5c4Cl)n3)c2)cn1] 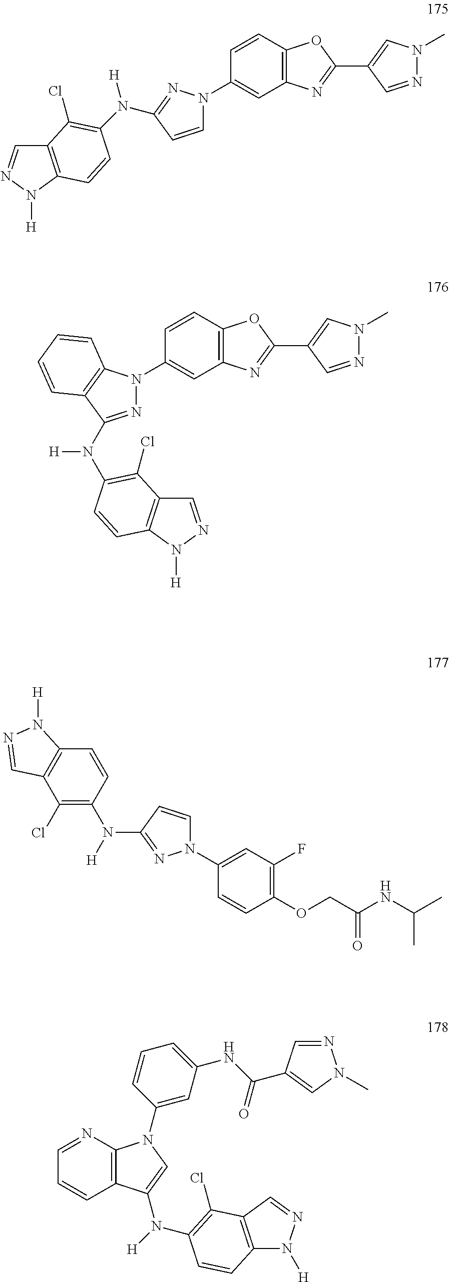 [SMILES: Cn1cc(-c2nc3cc(-n4ccc(Nc5ccc6[nH]ncc6c5Cl)n4)ccc3o2)cn1 ; Cn1cc(-c2nc3cc(-n4nc(Nc5ccc6[nH]ncc6c5Cl)c5ccccc54)ccc3o2)cn1 ; CC(C)NC(=O)COc1ccc(-n2ccc(Nc3ccc4[nH]ncc4c3Cl)n2)cc1F ; Cn1cc(C(=O)Nc2cccc(-n3cc(Nc4ccc5[nH]ncc5c4Cl)c4cccnc43)c2)cn1] 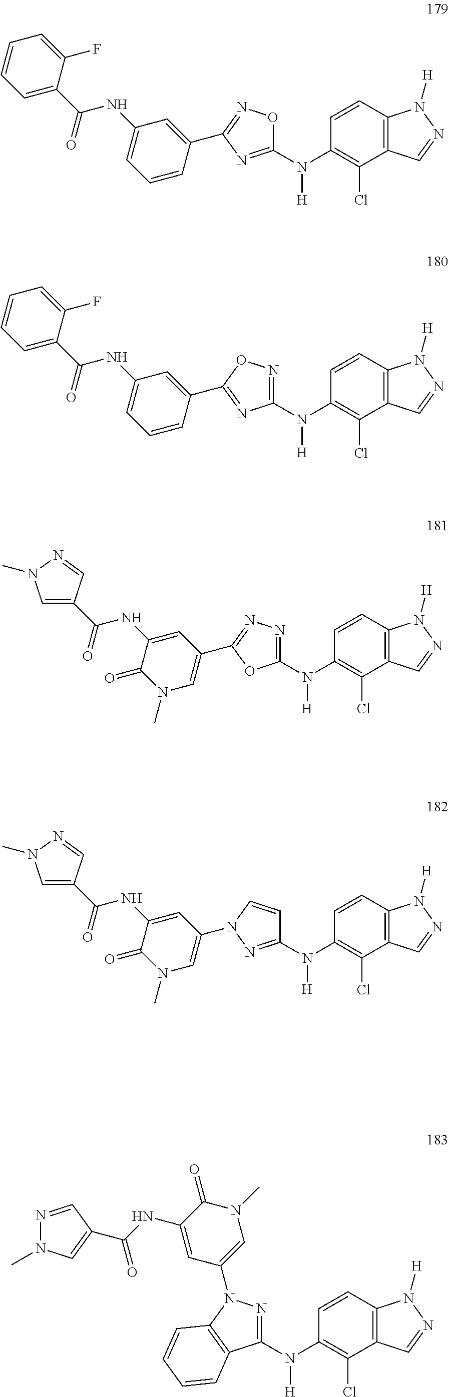 [SMILES: O=C(Nc1cccc(-c2noc(Nc3ccc4[nH]ncc4c3Cl)n2)c1)c1ccccc1F ; O=C(Nc1cccc(-c2nc(Nc3ccc4[nH]ncc4c3Cl)no2)c1)c1ccccc1F ; Cn1cc(C(=O)Nc2cc(-c3nnc(Nc4ccc5[nH]ncc5c4Cl)o3)cn(C)c2=O)cn1 ; Cn1cc(C(=O)Nc2cc(-n3ccc(Nc4ccc5[nH]ncc5c4Cl)n3)cn(C)c2=O)cn1 ; Cn1cc(C(=O)Nc2cc(-n3nc(Nc4ccc5[nH]ncc5c4Cl)c4ccccc43)cn(C)c2=O)cn1] 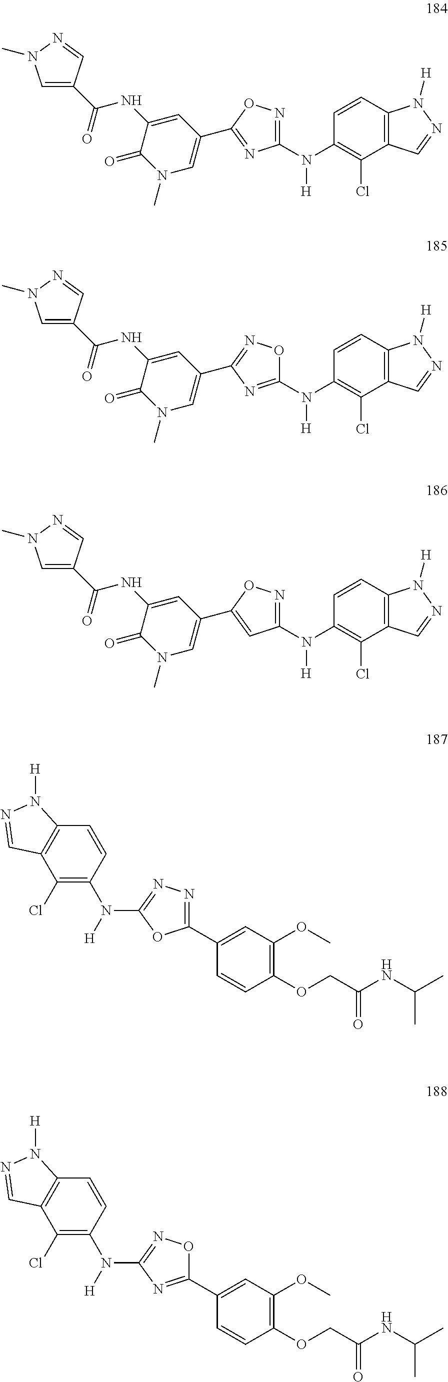 [SMILES: Cn1cc(C(=O)Nc2cc(-c3nc(Nc4ccc5[nH]ncc5c4Cl)no3)cn(C)c2=O)cn1 ; Cn1cc(C(=O)Nc2cc(-c3noc(Nc4ccc5[nH]ncc5c4Cl)n3)cn(C)c2=O)cn1 ; Cn1cc(C(=O)Nc2cc(-c3cc(Nc4ccc5[nH]ncc5c4Cl)no3)cn(C)c2=O)cn1 ; COc1cc(-c2nnc(Nc3ccc4[nH]ncc4c3Cl)o2)ccc1OCC(=O)NC(C)C ; COc1cc(-c2nc(Nc3ccc4[nH]ncc4c3Cl)no2)ccc1OCC(=O)NC(C)C] 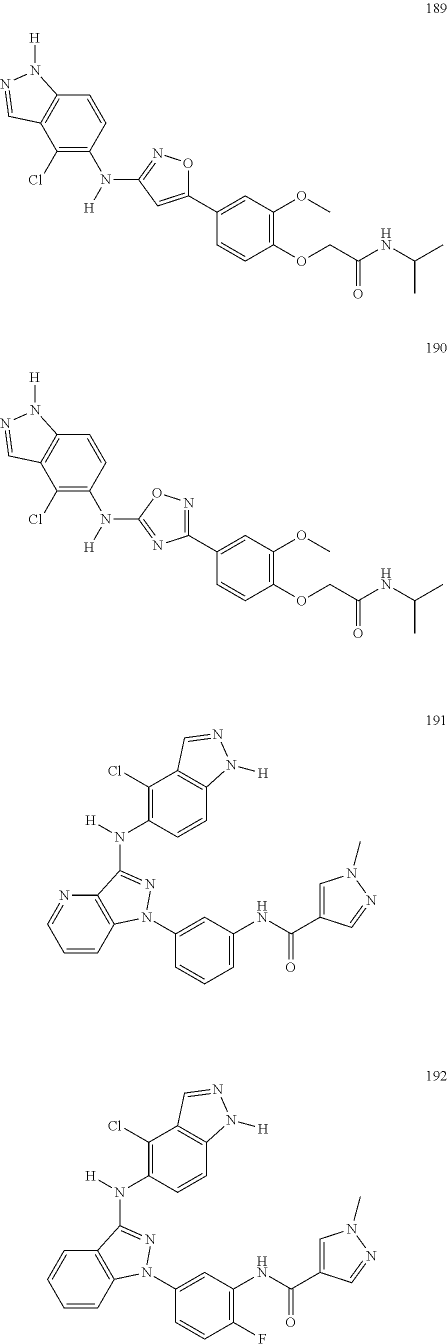 [SMILES: COc1cc(-c2cc(Nc3ccc4[nH]ncc4c3Cl)no2)ccc1OCC(=O)NC(C)C ; COc1cc(-c2noc(Nc3ccc4[nH]ncc4c3Cl)n2)ccc1OCC(=O)NC(C)C ; Cn1cc(C(=O)Nc2cccc(-n3nc(Nc4ccc5[nH]ncc5c4Cl)c4ncccc43)c2)cn1 ; Cn1cc(C(=O)Nc2cc(-n3nc(Nc4ccc5[nH]ncc5c4Cl)c4ccccc43)ccc2F)cn1] 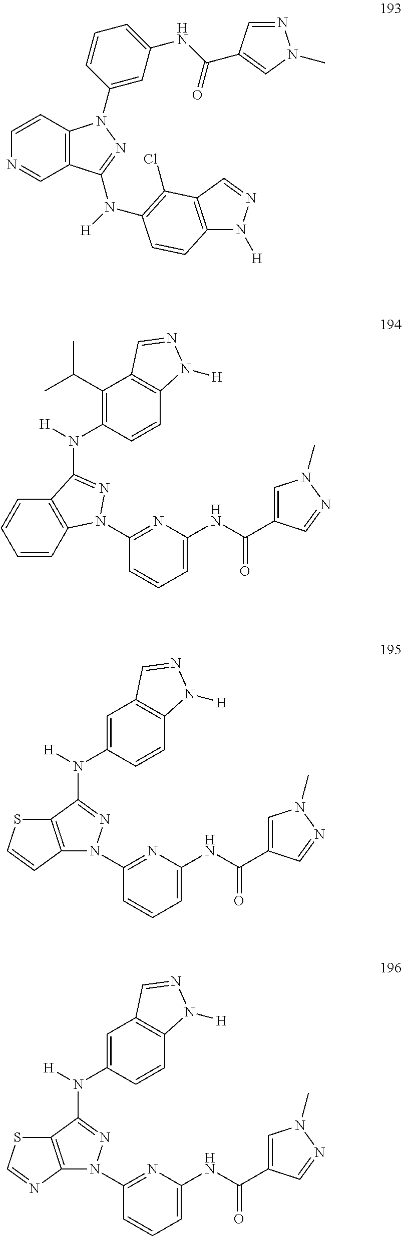 [SMILES: Cn1cc(C(=O)Nc2cccc(-n3nc(Nc4ccc5[nH]ncc5c4Cl)c4cnccc43)c2)cn1 ; CC(C)c1c(Nc2nn(-c3cccc(NC(=O)c4cnn(C)c4)n3)c3ccccc23)ccc2[nH]ncc12 ; Cn1cc(C(=O)Nc2cccc(-n3nc(Nc4ccc5[nH]ncc5c4)c4sccc43)n2)cn1 ; Cn1cc(C(=O)Nc2cccc(-n3nc(Nc4ccc5[nH]ncc5c4)c4scnc43)n2)cn1] 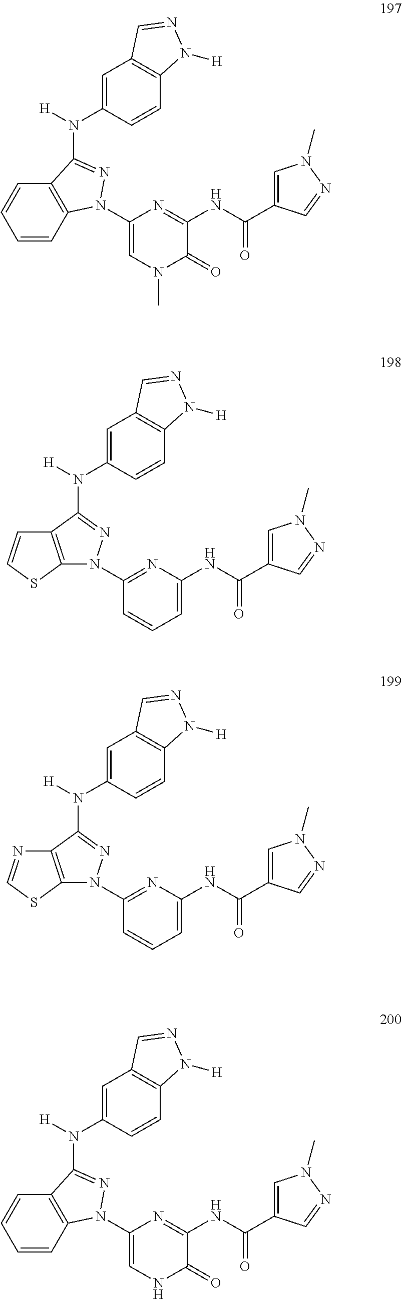 [SMILES: Cn1cc(C(=O)Nc2nc(-n3nc(Nc4ccc5[nH]ncc5c4)c4ccccc43)cn(C)c2=O)cn1 ; Cn1cc(C(=O)Nc2cccc(-n3nc(Nc4ccc5[nH]ncc5c4)c4ccsc43)n2)cn1 ; Cn1cc(C(=O)Nc2cccc(-n3nc(Nc4ccc5[nH]ncc5c4)c4ncsc43)n2)cn1 ; Cn1cc(C(=O)Nc2nc(-n3nc(Nc4ccc5[nH]ncc5c4)c4ccccc43)c[nH]c2=O)cn1] 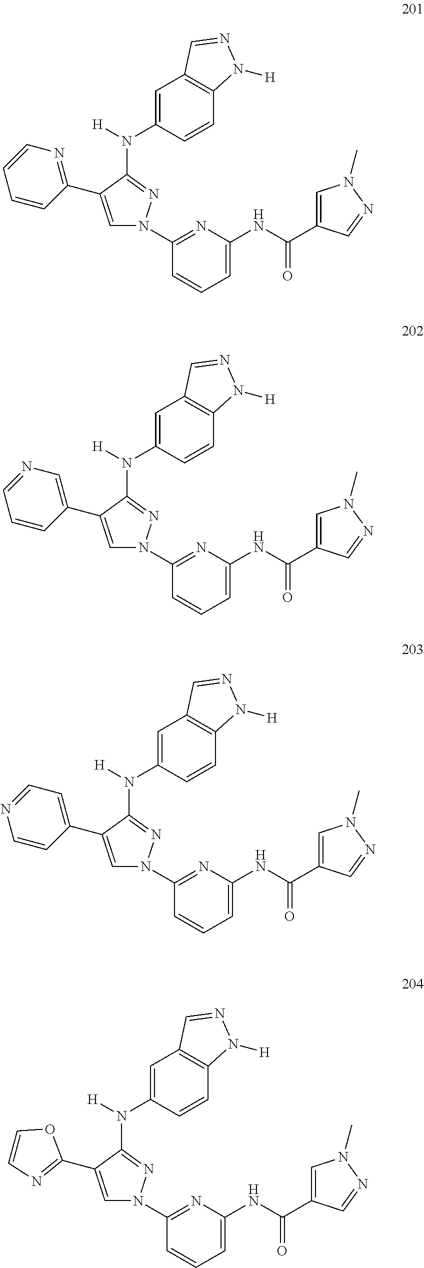 [SMILES: Cn1cc(C(=O)Nc2cccc(-n3cc(-c4ccccn4)c(Nc4ccc5[nH]ncc5c4)n3)n2)cn1 ; Cn1cc(C(=O)Nc2cccc(-n3cc(-c4cccnc4)c(Nc4ccc5[nH]ncc5c4)n3)n2)cn1 ; Cn1cc(C(=O)Nc2cccc(-n3cc(-c4ccncc4)c(Nc4ccc5[nH]ncc5c4)n3)n2)cn1 ; Cn1cc(C(=O)Nc2cccc(-n3cc(-c4ncco4)c(Nc4ccc5[nH]ncc5c4)n3)n2)cn1] 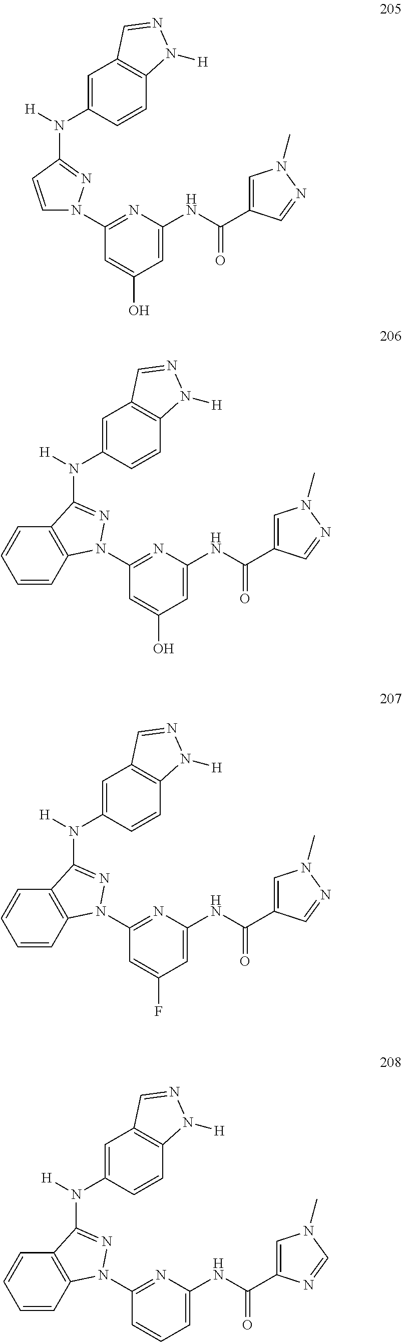 [SMILES: Cn1cc(C(=O)Nc2cc(O)cc(-n3ccc(Nc4ccc5[nH]ncc5c4)n3)n2)cn1 ; Cn1cc(C(=O)Nc2cc(O)cc(-n3nc(Nc4ccc5[nH]ncc5c4)c4ccccc43)n2)cn1 ; Cn1cc(C(=O)Nc2cc(F)cc(-n3nc(Nc4ccc5[nH]ncc5c4)c4ccccc43)n2)cn1 ; Cn1cnc(C(=O)Nc2cccc(-n3nc(Nc4ccc5[nH]ncc5c4)c4ccccc43)n2)c1] 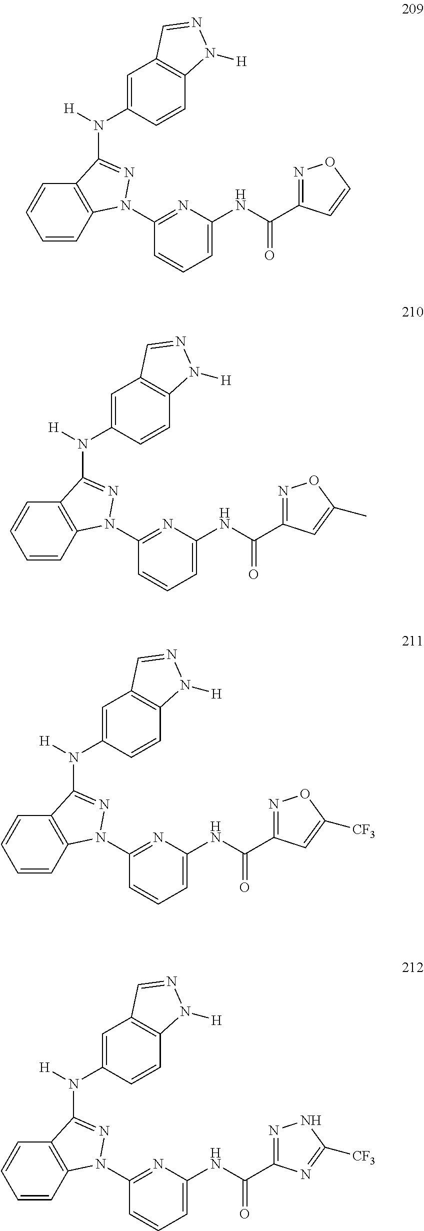 [SMILES: O=C(Nc1cccc(-n2nc(Nc3ccc4[nH]ncc4c3)c3ccccc32)n1)c1ccon1 ; Cc1cc(C(=O)Nc2cccc(-n3nc(Nc4ccc5[nH]ncc5c4)c4ccccc43)n2)no1 ; O=C(Nc1cccc(-n2nc(Nc3ccc4[nH]ncc4c3)c3ccccc32)n1)c1cc(C(F)(F)F)on1 ; O=C(Nc1cccc(-n2nc(Nc3ccc4[nH]ncc4c3)c3ccccc32)n1)c1n[nH]c(C(F)(F)F)n1] 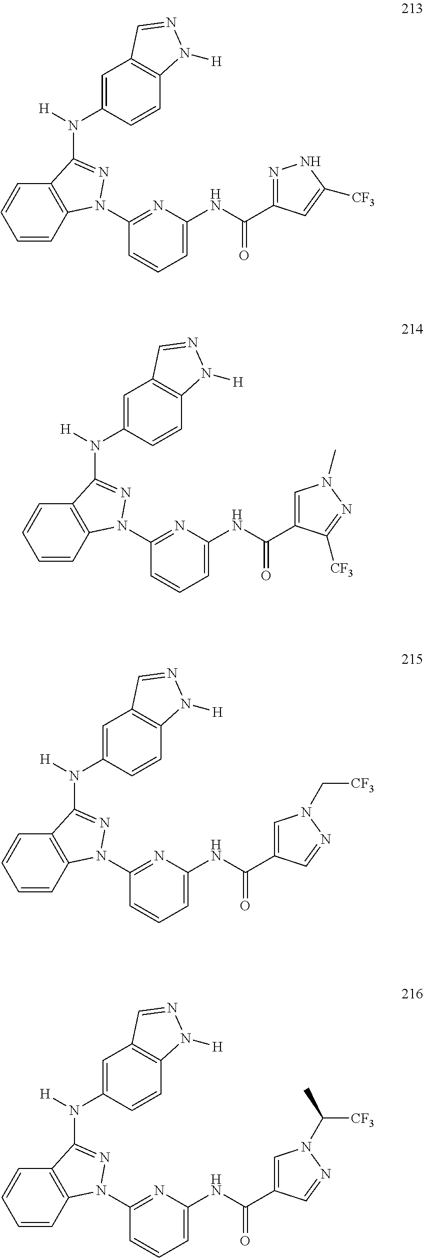 [SMILES: O=C(Nc1cccc(-n2nc(Nc3ccc4[nH]ncc4c3)c3ccccc32)n1)c1cc(C(F)(F)F)[nH]n1 ; Cn1cc(C(=O)Nc2cccc(-n3nc(Nc4ccc5[nH]ncc5c4)c4ccccc43)n2)c(C(F)(F)F)n1 ; O=C(Nc1cccc(-n2nc(Nc3ccc4[nH]ncc4c3)c3ccccc32)n1)c1cnn(CC(F)(F)F)c1 ; C[C@H](n1cc(C(=O)Nc2cccc(-n3nc(Nc4ccc5[nH]ncc5c4)c4ccccc43)n2)cn1)C(F)(F)F] 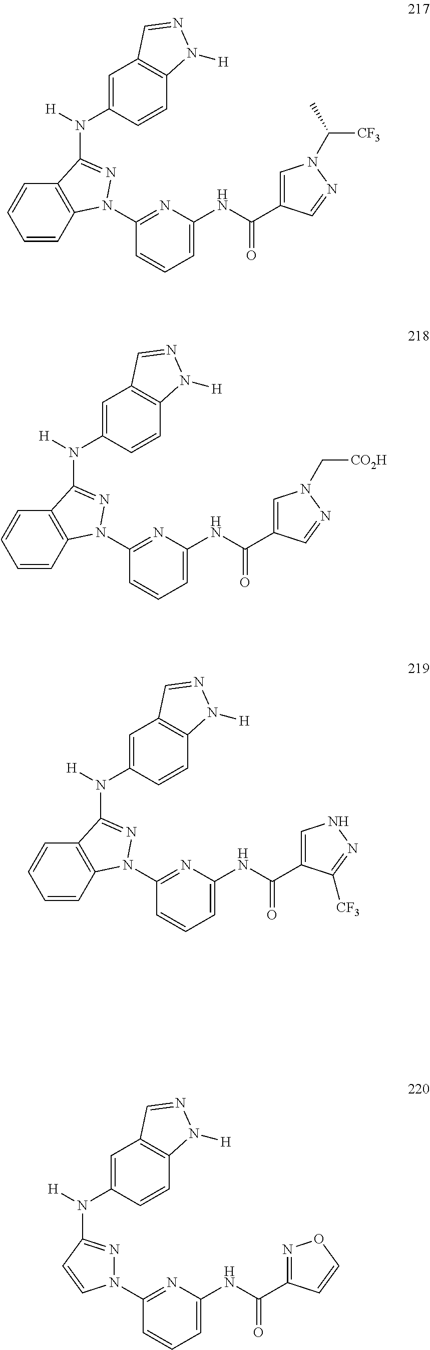 [SMILES: C[C@@H](n1cc(C(=O)Nc2cccc(-n3nc(Nc4ccc5[nH]ncc5c4)c4ccccc43)n2)cn1)C(F)(F)F ; O=C(O)Cn1cc(C(=O)Nc2cccc(-n3nc(Nc4ccc5[nH]ncc5c4)c4ccccc43)n2)cn1 ; O=C(Nc1cccc(-n2nc(Nc3ccc4[nH]ncc4c3)c3ccccc32)n1)c1c[nH]nc1C(F)(F)F ; O=C(Nc1cccc(-n2ccc(Nc3ccc4[nH]ncc4c3)n2)n1)c1ccon1] 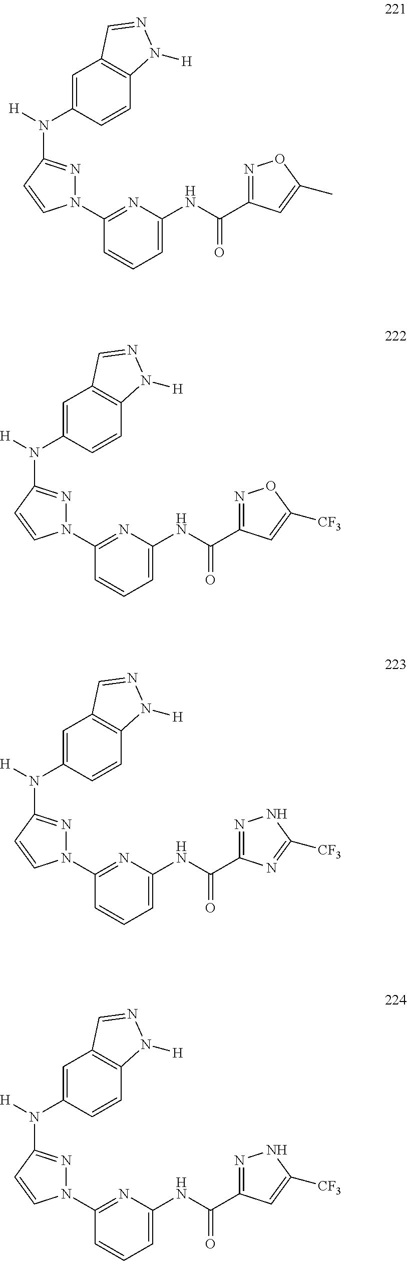 [SMILES: Cc1cc(C(=O)Nc2cccc(-n3ccc(Nc4ccc5[nH]ncc5c4)n3)n2)no1 ; O=C(Nc1cccc(-n2ccc(Nc3ccc4[nH]ncc4c3)n2)n1)c1cc(C(F)(F)F)on1 ; O=C(Nc1cccc(-n2ccc(Nc3ccc4[nH]ncc4c3)n2)n1)c1n[nH]c(C(F)(F)F)n1 ; O=C(Nc1cccc(-n2ccc(Nc3ccc4[nH]ncc4c3)n2)n1)c1cc(C(F)(F)F)[nH]n1] 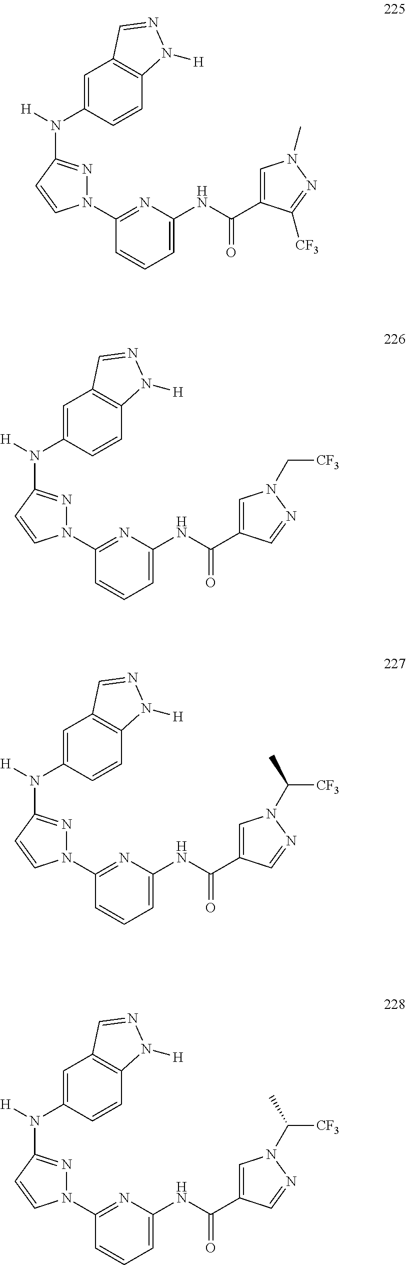 [SMILES: Cn1cc(C(=O)Nc2cccc(-n3ccc(Nc4ccc5[nH]ncc5c4)n3)n2)c(C(F)(F)F)n1 ; O=C(Nc1cccc(-n2ccc(Nc3ccc4[nH]ncc4c3)n2)n1)c1cnn(CC(F)(F)F)c1 ; C[C@H](n1cc(C(=O)Nc2cccc(-n3ccc(Nc4ccc5[nH]ncc5c4)n3)n2)cn1)C(F)(F)F ; C[C@@H](n1cc(C(=O)Nc2cccc(-n3ccc(Nc4ccc5[nH]ncc5c4)n3)n2)cn1)C(F)(F)F] 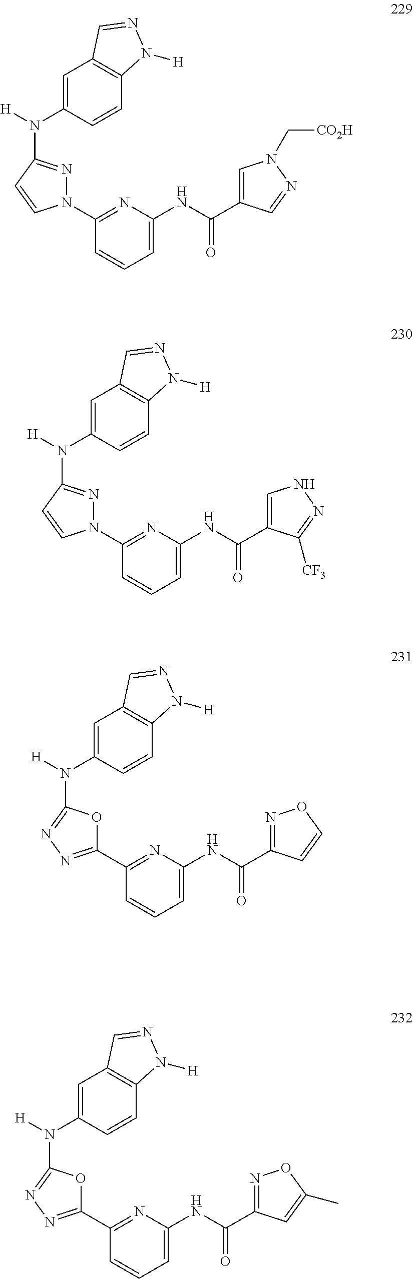 [SMILES: O=C(O)Cn1cc(C(=O)Nc2cccc(-n3ccc(Nc4ccc5[nH]ncc5c4)n3)n2)cn1 ; O=C(Nc1cccc(-n2ccc(Nc3ccc4[nH]ncc4c3)n2)n1)c1c[nH]nc1C(F)(F)F ; O=C(Nc1cccc(-c2nnc(Nc3ccc4[nH]ncc4c3)o2)n1)c1ccon1 ; Cc1cc(C(=O)Nc2cccc(-c3nnc(Nc4ccc5[nH]ncc5c4)o3)n2)no1] 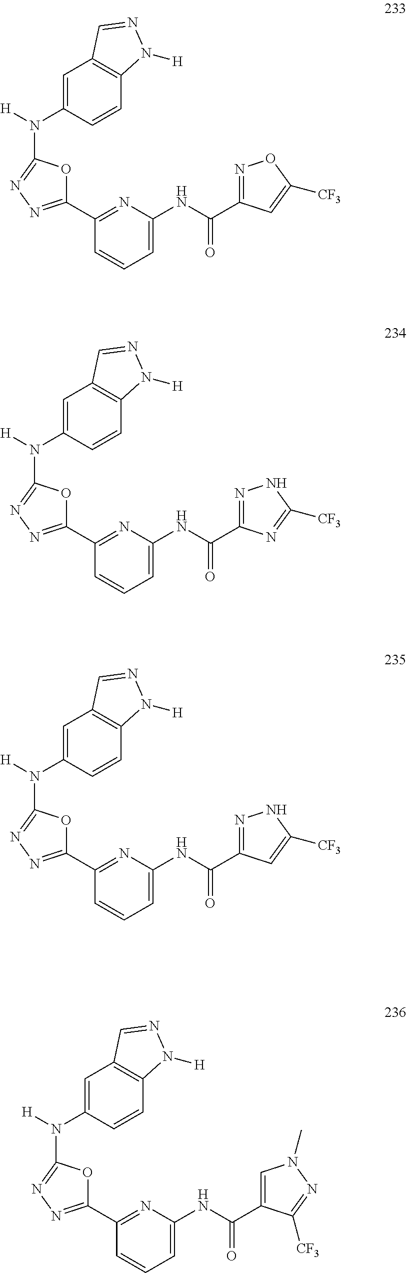 [SMILES: O=C(Nc1cccc(-c2nnc(Nc3ccc4[nH]ncc4c3)o2)n1)c1cc(C(F)(F)F)on1 ; O=C(Nc1cccc(-c2nnc(Nc3ccc4[nH]ncc4c3)o2)n1)c1n[nH]c(C(F)(F)F)n1 ; O=C(Nc1cccc(-c2nnc(Nc3ccc4[nH]ncc4c3)o2)n1)c1cc(C(F)(F)F)[nH]n1 ; Cn1cc(C(=O)Nc2cccc(-c3nnc(Nc4ccc5[nH]ncc5c4)o3)n2)c(C(F)(F)F)n1] 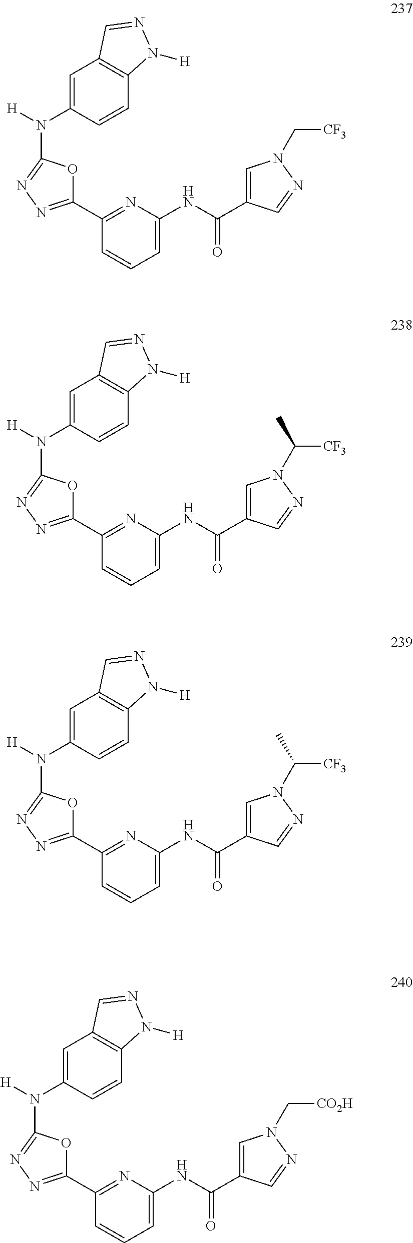 [SMILES: O=C(Nc1cccc(-c2nnc(Nc3ccc4[nH]ncc4c3)o2)n1)c1cnn(CC(F)(F)F)c1 ; C[C@H](n1cc(C(=O)Nc2cccc(-c3nnc(Nc4ccc5[nH]ncc5c4)o3)n2)cn1)C(F)(F)F ; C[C@@H](n1cc(C(=O)Nc2cccc(-c3nnc(Nc4ccc5[nH]ncc5c4)o3)n2)cn1)C(F)(F)F ; O=C(O)Cn1cc(C(=O)Nc2cccc(-c3nnc(Nc4ccc5[nH]ncc5c4)o3)n2)cn1] 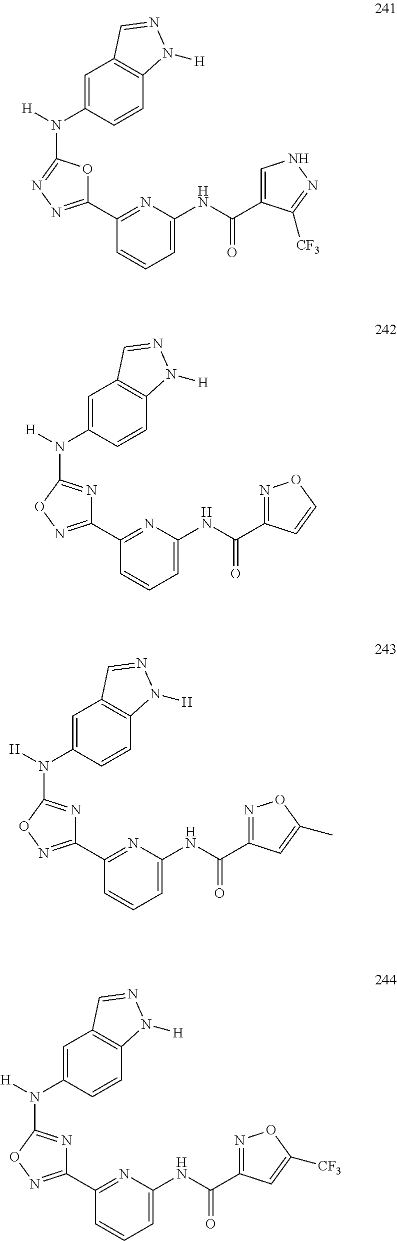 [SMILES: O=C(Nc1cccc(-c2nnc(Nc3ccc4[nH]ncc4c3)o2)n1)c1c[nH]nc1C(F)(F)F ; O=C(Nc1cccc(-c2noc(Nc3ccc4[nH]ncc4c3)n2)n1)c1ccon1 ; Cc1cc(C(=O)Nc2cccc(-c3noc(Nc4ccc5[nH]ncc5c4)n3)n2)no1 ; O=C(Nc1cccc(-c2noc(Nc3ccc4[nH]ncc4c3)n2)n1)c1cc(C(F)(F)F)on1] 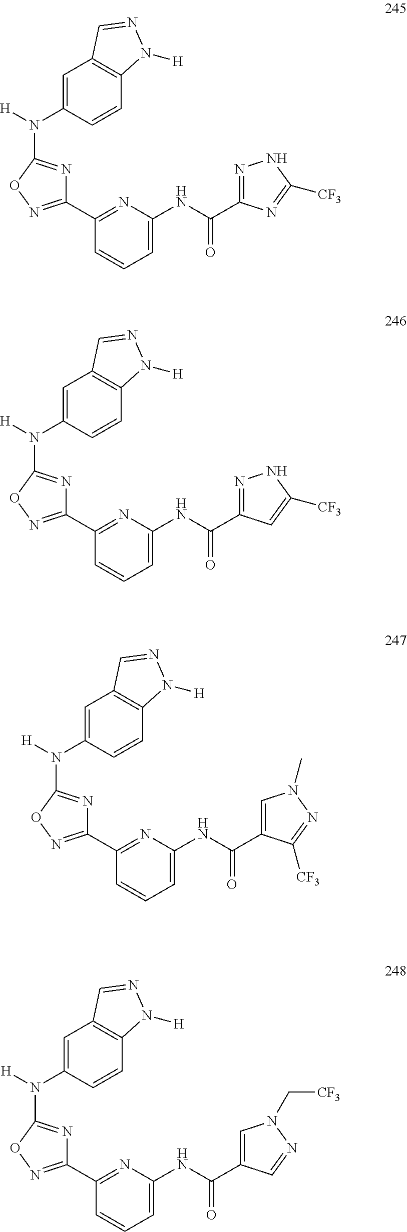 [SMILES: O=C(Nc1cccc(-c2noc(Nc3ccc4[nH]ncc4c3)n2)n1)c1n[nH]c(C(F)(F)F)n1 ; O=C(Nc1cccc(-c2noc(Nc3ccc4[nH]ncc4c3)n2)n1)c1cc(C(F)(F)F)[nH]n1 ; Cn1cc(C(=O)Nc2cccc(-c3noc(Nc4ccc5[nH]ncc5c4)n3)n2)c(C(F)(F)F)n1 ; O=C(Nc1cccc(-c2noc(Nc3ccc4[nH]ncc4c3)n2)n1)c1cnn(CC(F)(F)F)c1] 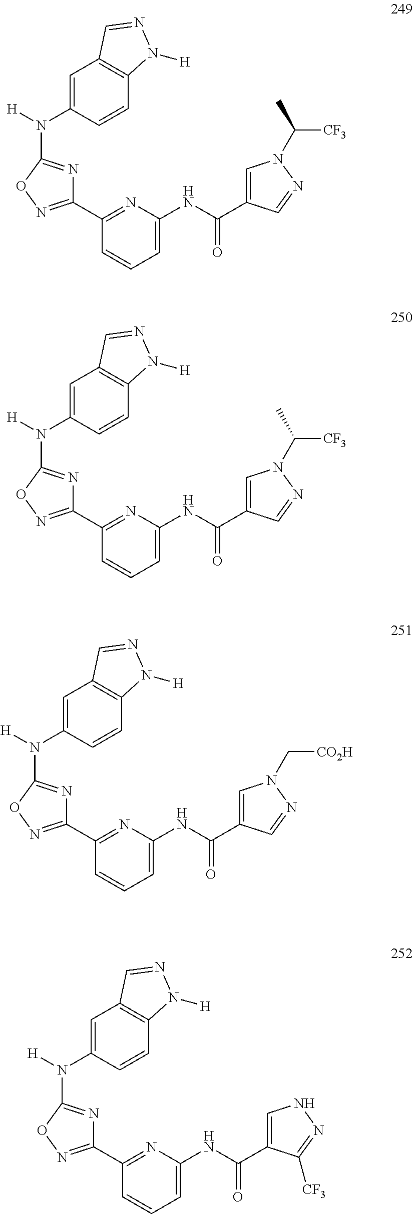 [SMILES: C[C@H](n1cc(C(=O)Nc2cccc(-c3noc(Nc4ccc5[nH]ncc5c4)n3)n2)cn1)C(F)(F)F ; C[C@@H](n1cc(C(=O)Nc2cccc(-c3noc(Nc4ccc5[nH]ncc5c4)n3)n2)cn1)C(F)(F)F ; O=C(O)Cn1cc(C(=O)Nc2cccc(-c3noc(Nc4ccc5[nH]ncc5c4)n3)n2)cn1 ; O=C(Nc1cccc(-c2noc(Nc3ccc4[nH]ncc4c3)n2)n1)c1c[nH]nc1C(F)(F)F] 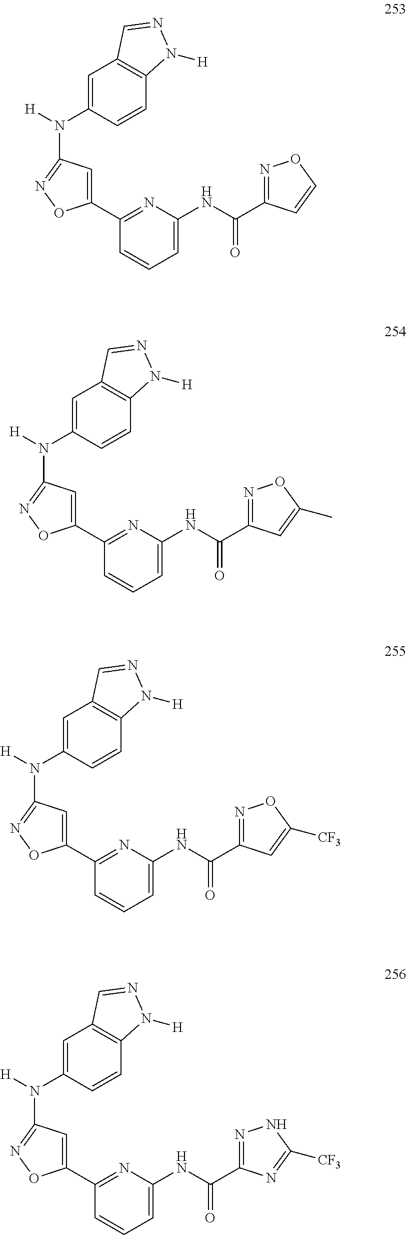 [SMILES: O=C(Nc1cccc(-c2cc(Nc3ccc4[nH]ncc4c3)no2)n1)c1ccon1 ; Cc1cc(C(=O)Nc2cccc(-c3cc(Nc4ccc5[nH]ncc5c4)no3)n2)no1 ; O=C(Nc1cccc(-c2cc(Nc3ccc4[nH]ncc4c3)no2)n1)c1cc(C(F)(F)F)on1 ; O=C(Nc1cccc(-c2cc(Nc3ccc4[nH]ncc4c3)no2)n1)c1n[nH]c(C(F)(F)F)n1] 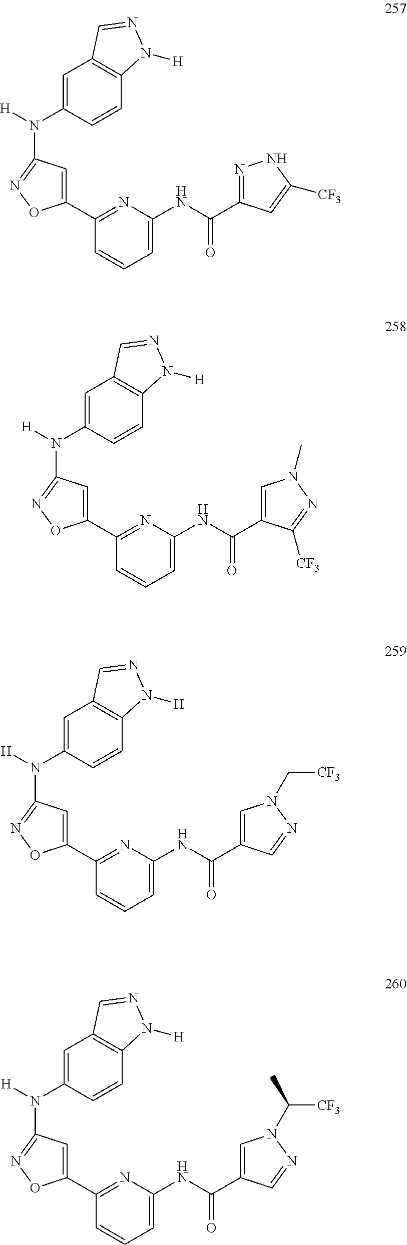 [SMILES: O=C(Nc1cccc(-c2cc(Nc3ccc4[nH]ncc4c3)no2)n1)c1cc(C(F)(F)F)[nH]n1 ; Cn1cc(C(=O)Nc2cccc(-c3cc(Nc4ccc5[nH]ncc5c4)no3)n2)c(C(F)(F)F)n1 ; O=C(Nc1cccc(-c2cc(Nc3ccc4[nH]ncc4c3)no2)n1)c1cnn(CC(F)(F)F)c1 ; C[C@H](n1cc(C(=O)Nc2cccc(-c3cc(Nc4ccc5[nH]ncc5c4)no3)n2)cn1)C(F)(F)F] 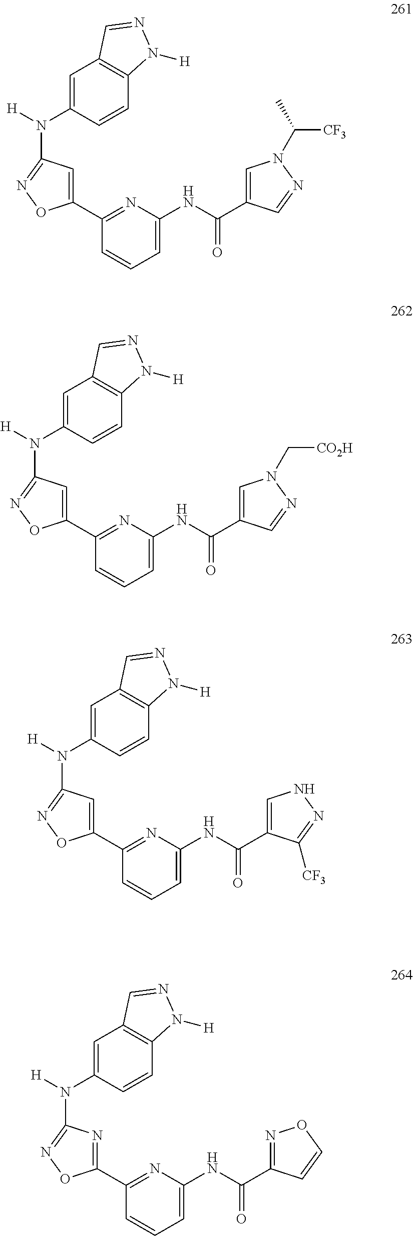 [SMILES: C[C@@H](n1cc(C(=O)Nc2cccc(-c3cc(Nc4ccc5[nH]ncc5c4)no3)n2)cn1)C(F)(F)F ; O=C(O)Cn1cc(C(=O)Nc2cccc(-c3cc(Nc4ccc5[nH]ncc5c4)no3)n2)cn1 ; O=C(Nc1cccc(-c2cc(Nc3ccc4[nH]ncc4c3)no2)n1)c1c[nH]nc1C(F)(F)F ; O=C(Nc1cccc(-c2nc(Nc3ccc4[nH]ncc4c3)no2)n1)c1ccon1] 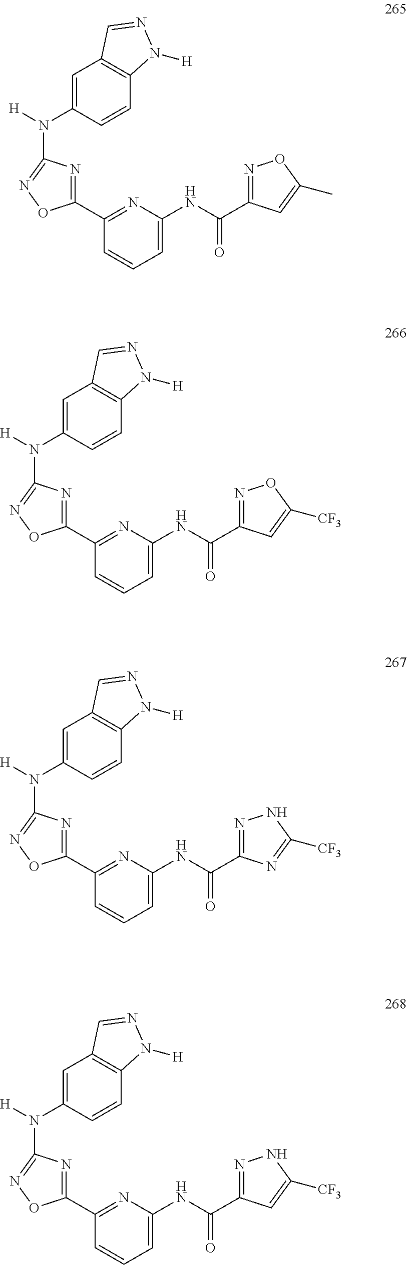 [SMILES: Cc1cc(C(=O)Nc2cccc(-c3nc(Nc4ccc5[nH]ncc5c4)no3)n2)no1 ; O=C(Nc1cccc(-c2nc(Nc3ccc4[nH]ncc4c3)no2)n1)c1cc(C(F)(F)F)on1 ; O=C(Nc1cccc(-c2nc(Nc3ccc4[nH]ncc4c3)no2)n1)c1n[nH]c(C(F)(F)F)n1 ; O=C(Nc1cccc(-c2nc(Nc3ccc4[nH]ncc4c3)no2)n1)c1cc(C(F)(F)F)[nH]n1] 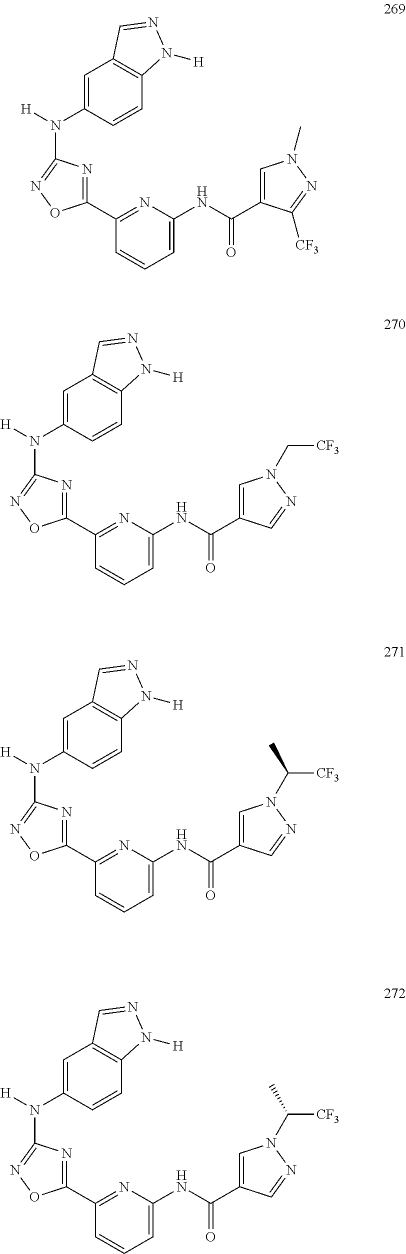 [SMILES: Cn1cc(C(=O)Nc2cccc(-c3nc(Nc4ccc5[nH]ncc5c4)no3)n2)c(C(F)(F)F)n1 ; O=C(Nc1cccc(-c2nc(Nc3ccc4[nH]ncc4c3)no2)n1)c1cnn(CC(F)(F)F)c1 ; C[C@H](n1cc(C(=O)Nc2cccc(-c3nc(Nc4ccc5[nH]ncc5c4)no3)n2)cn1)C(F)(F)F ; C[C@@H](n1cc(C(=O)Nc2cccc(-c3nc(Nc4ccc5[nH]ncc5c4)no3)n2)cn1)C(F)(F)F] 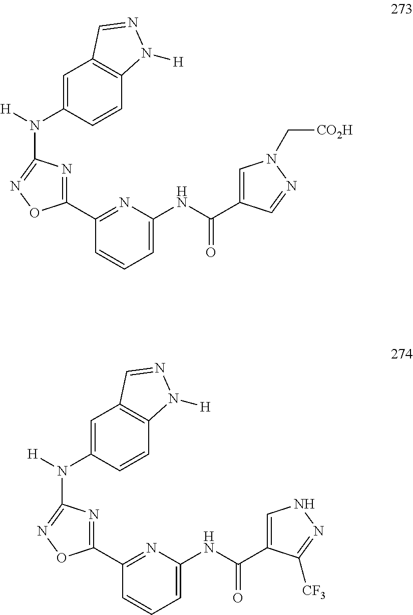 [SMILES: O=C(O)Cn1cc(C(=O)Nc2cccc(-c3nc(Nc4ccc5[nH]ncc5c4)no3)n2)cn1 ; O=C(Nc1cccc(-c2nc(Nc3ccc4[nH]ncc4c3)no2)n1)c1c[nH]nc1C(F)(F)F]
or a pharmaceutically acceptable salt, co-crystal, tautomer, stereoisomer, solvate, hydrate, polymorph, isotopically enriched compound, or prodrug thereof.

133. The compound of any of claims 1-132, or a pharmaceutically acceptable salt or tautomer thereof.

134. A pharmaceutical composition comprising a compound of any of claims 1-132, or a pharmaceutically acceptable salt, co-crystal, tautomer, stereoisomer, solvate, hydrate, polymorph, isotopically enriched compound, or prodrug thereof, and a pharmaceutically acceptable excipient.

135. A method of treating a disease or disorder associated with ROCK2 in a subject in need thereof, the method comprising administering to the subject an effective amount of a compound of any of claims 1-132, or pharmaceutically acceptable salt, co-crystal, tautomer, stereoisomer, solvate, hydrate, polymorph, isotopically enriched compound, or prodrug thereof, or a pharmaceutical composition of claim 134.

136. A method of preventing a disease or disorder associated with ROCK2 in a subject in need thereof, the method comprising administering to the subject an effective amount of a compound of any of claims 1-132, or pharmaceutically acceptable salt, co-crystal, tautomer, stereoisomer, solvate, hydrate, polymorph, isotopically enriched compound, or prodrug thereof, or a pharmaceutical composition of claim 134.

137. A compound of any one of claims 1-132, or a pharmaceutically acceptable salt, co-crystal, tautomer, stereoisomer, solvate, hydrate, polymorph, isotopically enriched compound, or prodrug thereof, or a pharmaceutical composition of claim 134, for use in the treatment of a disease or disorder associated with ROCK2.

138. A compound of any one of claims 1-132, or a pharmaceutically acceptable salt, co-crystal, tautomer, stereoisomer, solvate, hydrate, polymorph, isotopically enriched compound, or prodrug thereof, or a pharmaceutical composition of claim 134, for use in the prevention of a disease or disorder associated with ROCK2.

139. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is a fibrotic disorder, autoimmune disease, inflammatory condition, edema, opthalmic disease, cardiovascular disease, central nervous system disorder, or cancer.

140. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is pulmonary fibrosis, cystic pulmonary fibrosis, idiopathic pulmonary fibrosis, radiation induced lung injury, liver fibrosis including cirrhosis, cardiac fibrosis including arterial fibrosis, endomyocardial fibrosis, old myocardial infraction, arterial stiffness, atherosclerosis, restenosis, arthrofibrosis, Crohns disease, myelofibrosis, Peyronie's diseases, nephrogenic systemic fibrosis, progressive massive fibrosis, retroperitoneal cavity fibrosis, schleroderma/systemic sclerosis, mediastinal fibrosis, Keloids and hypertrophic scars, glial scaring, or renal fibrosis.

141. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is Huntington's disease, Parkinson's disease, Alzheimer's disease, Amyotrophic lateral sclerosis (ALS), Batten disease, dementia, spinal muscular atrophy, motor neurone diseases, spinocerebellar ataxia, acute or chronic pain, dementia, neuronal degeneration, spinal cord injury, or cerebral vasospasm.

142. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is glaucoma.

143. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is asthma, cardiovascular inflammation, renal inflammation, or arteriosclerosis.

144. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is hypertension, atherosclerosis, angina, arterial obstruction, peripheral arterial disease, peripheral circulatory disorder, cerebral cavernous malformation, restenosis, cardiac hypertrophy, ocular hypertension, cerebral ischemia, cerebral vasospasm, acute respiratory distress syndrome (ARDS), or erectile dysfunction.

145. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is rheumatoid arthritis, multiple sclerosis, systemic lupus erythematosus, psoriasis, Crohn's disease, atopic dermatitis, eczema, or graft-versus-host disease (GVHD).

146. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is cancer.

147. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is lymphedema.

148. The method of claim 147, or the compound, or pharmaceutically acceptable salt, co-crystal, tautomer, stereoisomer, solvate, hydrate, polymorph, isotopically enriched compound, or prodrug thereof, or pharmaceutical composition for use of claim 147, wherein the lymphedema is caused at least by a parasitic disease.

149. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is angioedema, brain edema, CHAPLE syndrome, cardiac edema, hydrops fetalis, inflammatory edema, macular edema, myxedema, pulmonary edema, peripheral edema, periorbital edema, or cutaneous edema.

150. The method of claim 135 or 136, or the compound, or pharmaceutically acceptable salt, co-crystal, tautomer, stereoisomer, solvate, hydrate, polymorph, isotopically enriched compound, or prodrug thereof, or pharmaceutical composition for use of claim 137 or 138, wherein the disease or disorder associated with ROCK2 is hereditary angioedema, cystoid macular edema, Irvine-Gass syndrome, diabetic macular edema, or pedal edema.

151. A method of inhibiting ROCK2 comprising contacting ROCK2 with an effective amount of a compound of any of claims 1-132, or pharmaceutically acceptable salt, co-crystal, tautomer, stereoisomer, solvate, hydrate, polymorph, isotopically enriched compound, or prodrug thereof, or a pharmaceutical composition of claim 134.

152. The method of claim 151, wherein the ROCK2 is in vitro.

153. A method of screening a library of compounds comprising performing an assay on a compound of any of claims 1-132, or pharmaceutically acceptable salt, co-crystal, tautomer, stereoisomer, solvate, hydrate, polymorph, isotopically enriched compound, or prodrug thereof, and an additional compound, wherein the additional compound is different from the compound of any of claims 1-132, or pharmaceutically acceptable salt, co-crystal, tautomer, stereoisomer, solvate, hydrate, polymorph, isotopically enriched compound, or prodrug thereof.

154. A kit comprising a compound of any of claims 1-132, or a pharmaceutically acceptable salt, co-crystal, tautomer, stereoisomer, solvate, hydrate, polymorph, isotopically enriched compound, or prodrug thereof, or a pharmaceutical composition of claim 134, and instructions for using the compound, or pharmaceutically acceptable salt, co-crystal, tautomer, stereoisomer, solvate, hydrate, polymorph, isotopically enriched compound, or prodrug thereof, or pharmaceutical composition.
